(19) United States
(12) Patent Application Publication
(10) Pub. No.: US 2025/0136650 A1
(43) Pub. Date: May 1, 2025

(54) COMPOSITIONS AND METHODS FOR DNA BINDING AND TRANSCRIPTIONAL REGULATION

(71) Applicant: THE UNIVERSITY OF CHICAGO, Chicago, IL (US)

(72) Inventors: Raymond MOELLERING, Chicago, IL (US); Zeyu QIAO, Chicago, IL (US); Scott OAKES, Chicago, IL (US); Shannon ELF, Chicago, IL (US); Marsha ROSNER, Chicago, IL (US); Chi Long NGUYEN, Chicago, IL (US)

(73) Assignee: THE UNIVERSITY OF CHICAGO, Chicago, IL (US)

(21) Appl. No.: 18/713,104

(22) PCT Filed: Nov. 23, 2022
(86) PCT No.: PCT/US2022/080387

§ 371 Date: May 23, 2024

Related U.S. Application Data
(60) Provisional application No. 63/282,647, filed on Nov. 23, 2021.

(51) Int. Cl. C07K 14/47; A61K 38/00; A61K 45/06; A61P 35/00

(52) U.S. Cl. CPC C07K 14/4702; A61K 45/06; A61P 35/00; A61K 38/00; C07K 2319/80

(57) ABSTRACT

Aspects of the present disclosure are directed to synthetic DNA binding peptides, as well as methods of generating such peptides and methods for use of such peptides in, for example, DNA binding, modifying gene expression, and treatment of various conditions such as cancer, fibrosis, and diabetes. Certain aspects provide synthetic DNA binding dimers comprising two modified bZIP peptides, each comprising a modified basic domain and a modified leucine zipper domain and linked via an interpeptide linker (e.g., a side-by-side interpeptide linker). Also disclosed are universal methods for generating high affinity synthetic DNA binding dimers from any bZIP protein.

### CROSS-REFERENCE TO RELATED APPLICATIONS

This application claims the benefit of priority of U.S. Provisional Patent Application No. 63/282,647 filed Nov. 23, 2021, which is hereby incorporated by reference in its entirety.

### SEQUENCE LISTING

The instant application contains a Sequence Listing which has been submitted in XML format and is hereby incorporated by reference in its entirety. Said XML copy, created on Feb. 3, 2023, is named “ARCD_P0754WO_SL” and is 201 kilobytes in size.

### BACKGROUND

### I. Field of the Invention

Aspects of this invention relate at least to the fields of synthetic chemistry, structural biology, and molecular biology. In some particular aspects, the invention can include engineered DNA-binding dimers that include at least first and second bZIP proteins having specific modifications that the increase stability of the dimers and/or increase binding affinity of the dimers to target DNA sequences.

### II. Background

Basic leucine zipper (bZIP)-containing transcription factors (zTFs) are powerful proteins that turn specific genes “on” or “off” by binding to nearby DNA. By controlling gene expression, zTFs have a major influence on cell behavior, such as whether cells grow or die, and as such are overexpressed or deregulated in the majority of cancers. Despite the profound need for targeted therapies against zTFs, they remain largely untapped as drug targets due to the challenges of targeting protein-DNA interactions.

Transcription factors (TFs) control gene expression and cellular state by binding and recruiting regulatory complexes to specific DNA promoter and enhancer sequences in the genome. Dysregulation of TF activity is causal in the initiation and progression of cancer and many other diseases^{1,2}. Despite their biological validation as some of the most direct and effective targets for cancer treatment³, the majority of TFs remain untapped as drug targets due to the challenges of targeting protein-protein and protein-DNA interactions. Moreover, targeting one TF alone may be insufficient to turn off a disease-associated transcriptional program, owing to overlapping regulation of genes by several TFs.

Two archetypal, and as-yet untargeted oncogenic TFs are X-box binding protein 1 (XBP1) and hypoxia-inducible factor 1α (HIF1α). Both XBP1 and HIF1α are activated in cancer cells by changes in nutrient availability, hyperactive metabolism and hypoxia in the tumor microenvironment, which causes them to form complexes at canonical DNA motifs, such as the unfolded protein response element (UPRE)⁴ and hypoxia-induced response element (HRE),^{5,6} respectively. These operator motifs exist upstream of an array of target genes that promote tumor cell survival, proliferation and therapeutic resistance. HIF1α is widely implicated in driving malignant phenotypes, including drug resistance and metastasis, in essentially all solid tumors⁷⁻⁹. Likewise, the spliced, active form of XBP1 (referred to as XBP1s) is mutated, overexpressed or activated in numerous solid and blood cancers¹⁻¹². Intriguingly, an increasing body of evidence suggests that XBP1 and HIF1α, which belong to distinct basic leucine zipper (bZIP) and basic helix-loop-helix (bHLH) TF families and have not been shown to directly interact with one another 13,14 may bind and coregulate a subset of hypoxia-responsive genes¹⁵. This signature is particularly prevalent in triple negative breast cancer (TNBC), where XBP1 and HIF1α are strongly upregulated, correlate with poor patient outcomes, and are required for tumor cell growth and survival in preclinical models of this cancer^{7,16,17}. In principle, TF mimetics that can bind UPRE and/or HRE DNA sequences inside of cells could prevent the recruitment of XBP1 and HIF1α to target genes and subsequent activation of oncogenic pathways and phenotypes.

There exists a need for specific, high affinity transcription factor mimetics capable of competing for zTF binding to DNA, as well as methods for use of such compositions in research and therapeutic applications.

### SUMMARY

The present disclosure addresses certain needs by providing high affinity DNA-binding molecules capable of competing for zTF (e.g., Fos/Jun, XBP1, ATF4, CEBPβ, etc.) binding and of reducing expression of zTF target genes. In one aspect of the present invention, the inventors have discovered that certain modifications can be made to bZIP dimers that allow for increased efficacy and/or stability of the dimers. Non-limiting examples of such modifications can include (1) the use of intrapeptide stabilizing linkages in the bZIP dimers, (2) linkages between first and second bZIP proteins, and/or (3) certain amino acid substitutions made to the first and/or second bZIP proteins. An example of such a modification can include amino acid substitutions made to the leucine zipper domain sequences of the first and second bZIP proteins that form enginerred dimers of the present invention. In one example, such amino acid substitutions can include, any one of, any combination of, or all of the following substitutions: (i) each of the first and second bZIP proteins, individually, have an isoleucine, leucine, or valine at position “a” of their respective leucine zipper domain sequences; (ii) each of the first and second bZIP proteins, individually, have an isoleucine, leucine, or valine at position “d” of their respective leucine zipper domain sequences; (iii) each of the first and second bZIP proteins, individually, have a leucine at position “a” of their respective leucine zipper domain sequences; (iv) each of the first and second bZIP proteins, individually, have an isoleucine at position “a” of their respective leucine zipper domain sequences; each of the first and second bZIP proteins, individually, have a leucine at position “d” of their respective leucine zipper domain sequences; (v) each of the first and second bZIP proteins, individually, have an isoleucine at position “d” of their respective leucine zipper domain sequences; (vi) a glutamine is present at position “e” of the leucine zipper domain sequence of the first bZIP protein and a glutamine is present at position “g” of the leucine zipper domain sequence of the second bZIP protein, or an arginine is present at position “e” of the leucine zipper domain sequence of the first bZIP protein and a glutamic acid is present at position “g” of the leucine zipper domain sequence of the second bZIP protein; (vii) an arginine is present at position “e” of the leucine zipper domain sequence of the second bZIP protein and a glutamic acid is present at position “g” of the leucine zipper domain sequence of the first bZIP protein; and/or (viii) at least one or both of the leucine zipper domain sequences of the first and/or second bZIP proteins have an alanine at least at one or more of positions “b”, “c”, or “f”. With out wishing to be bound by theory, and as illustrated in non-limiting embodiments in the Examples, modifications (1), (2), and/or (3) are believed to increase the stability of the DNA-binding dimers of the present invention and/or increase the binding affinity of the DNA-binding dimers of the present invention to target DNA sequences. Increased stability and/or increased binding affinity can result in more effective dimers for therapeutic uses.

Also disclosed are methods for design and synthesis of such high affinity DNA-binding molecules starting from any natural zTF as a template. Engineered peptides useful as, for example, precursors in synthesis of DNA-binding molecules are also described herein. Further disclosed are methods for use of the disclosed DNA-binding molecules in various applications, including modifying gene expression and treatment of various conditions (e.g., cancer, fibrosis, diabetes). Certain aspects are directed to use of the disclosed DNA-binding molecules for targeting XBP1 and/or HIF1α for treatment of cancer such as triple negative breast cancer (TNBC).

Aspects of the present disclosure include engineered DNA-binding dimers, bZIP transcriptional repressors, engineered peptides, pharmaceutical compositions, methods for designing engineered peptides, methods for synthesizing engineered peptides, methods for designing engineered DNA-binding dimers, methods for synthesizing engineered DNA-binding dimers, methods for introducing engineered DNA-binding dimers into a cell, methods for altering gene expression. Engineered DNA-binding dimers of the disclosure can include at least 1, 2, 3, 4, or more of the following: an engineered peptide, an interpeptide linker, a modified basic domain sequence, a modified leucine zipper domain sequence, a non-natural amino acid, an intramolecular helix stabilizing linker, and a intrapeptide stabilizing linkage. Any one or more of the preceding components may be excluded in certain aspects. Methods of the present disclosure can include at least 1, 2, 3, 4, 5, or more of the following steps: obtaining a sequence of a bZIP protein, identifying a basic domain of a bZIP protein, identifying a leucine zipper domain of a bZIP protein, designing an engineered peptide, synthesizing an engineered peptide, synthesizing an engineered DNA-binding dimer, introducing an engineered DNA-binding dimer into a cell, culturing a cell with an engineered DNA-binding dimer, and administering a composition comprising an engineered DNA-binding dimer to a subject. Any one or more of the preceding steps may be excluded in aspects of the disclosure.

Disclosed herein, in some aspects, is an engineered DNA-binding dimer comprising (a) a first engineered peptide comprising (i) a basic domain sequence of a first bZIP protein and (ii) a leucine zipper domain sequence of the first bZIP protein; and (b) a second engineered peptide linked to the first engineered peptide via a side-by-side interpeptide linkage, the second engineered peptide comprising (i) a basic domain sequence of a second bZIP protein and (ii) a leucine zipper domain sequence of the second bZIP protein. In some aspects, the engineered peptides can be modified by: (1) introducing intrapeptide stabilizing linkages in the first and/or second bZIP proteins (e.g., linkages can be included in the basic domain and/or the leucine zipper domain sequences, preferably the basic domain sequences, of the first and/or second bZIP proteins; (2) introducing specific linker molecules to link together (e.g., covalent bond) the first and second bZIP proteins and/or where the linker molecules link together the first and second bZIP proteins; and/or (3) introducing amino acid substitutions into the first and/or second bZIP proteins. In one aspects, the engineered DNA-binder dimers of the present invention can be modified such that each of the first and second bZIP proteins, individually, have an isoleucine, leucine, or valine at position “a” of their respective leucine zipper domain sequences, and/or each of the first and second bZIP proteins, individually, have an isoleucine, leucine, or valine at position “d” of their respective leucine zipper domain sequences. In some aspects, the engineered DNA-binding dimer can have a modification such that each of the first and second bZIP proteins, individually, have a leucine at position “a” of their respective leucine zipper domain sequences. In other aspects, the engineered DNA-binding dimer can have a modification such that each of the first and second bZIP proteins, individually, have an isoleucine at position “a” of their respective leucine zipper domain sequences. In some aspects, each of the first and second bZIP proteins, individually, have a leucine at position “d” of their respective leucine zipper domain sequences. In other aspects, each of the first and second bZIP proteins, individually, have an isoleucine at position “d” of their respective leucine zipper domain sequences. In yet another aspect, the engineered DNA-binding dimers of the present invention can include a glutamine at position “e” of the leucine zipper domain sequence of the first bZIP protein and a glutamine at position “g” of the leucine zipper domain sequence of the second bZIP protein or an arginine at position “e” of the leucine zipper domain sequence of the first bZIP protein and a glutamic acid at position “g” of the leucine zipper domain sequence of the second bZIP protein. In yet another aspects, the engineered DNA-binding dimers of the present invention can include an arginine at position “e” of the leucine zipper domain sequence of the second bZIP protein and a glutamic acid at position “g” of the leucine zipper domain sequence of the first bZIP protein. In some aspects, the engineered DNA-binding dimers of the present invention can be modified such that at least one or both of the leucine zipper domain sequences of the first and/or second bZIP proteins have an alanine at least at one or more of positions “b”, “c”, or “f”.

In some aspects, the modified basic domain sequence of the first bZIP protein is at most, at least, or exactly 30, 29, 28, 27, 26, 25, 24, 23, 22, 21, 20, 19, 18, 17, 16, or 15 residues in length. In some aspects, the modified basic domain sequence of the first bZIP protein is at most 25 residues in length. In some aspects, the modified basic domain sequence of the first bZIP protein is 20 residues in length. The modified basic domain sequence may have at least 60%, 65%, 70%, 75%, 80%, 85%, 90%, or 95% identity with the basic domain of the first bZIP protein. The modified basic domain sequence may comprise 5, 6, 7, 8, 9, 10, 11, 12, 13, 14, 15, 16, 17, 18, 19, 20, 21, 22, 23, 24, or 25 residues from the basic domain of the first bZIP protein. In some aspects, the modified leucine zipper domain sequence of the first bZIP protein is at most, at least, or exactly 25, 24, 23, 22, 21, 20, 19, 18, 17, 16, 15, 14, 13, 12, 11, 10, 9, or 8 residues in length. In some aspects, the modified leucine zipper domain sequence of the first bZIP protein is at most 15 residues in length. In some aspects, the modified leucine zipper domain sequence of the first bZIP protein is 12 residues in length. The modified leucine zipper binding domain sequence may have at least 40%, 45%, 50%, 55%, 60%, 65%, 70%, 75%, 80%, 85%, 90%, or 95% identity with the leucine zipper domain of the first bZIP protein. The modified leucine zipper domain sequence may comprise 5, 6, 7, 8, 9, 10, 11, 12, 13, 14, 15, 16, 17, 18, 19, 20, 21, 22, 23, 24, or 25 residues from the leucine zipper domain of the first bZIP protein.

In some aspects, the modified basic domain sequence of the second bZIP protein is at most, at least, or exactly 30, 29, 28, 27, 26, 25, 24, 23, 22, 21, 20, 19, 18, 17, 16, or 15 residues in length. In some aspects, the modified basic domain sequence of the second bZIP protein is at most 25 residues in length. In some aspects, the modified basic domain sequence of the second bZIP protein is 20 residues in length. The modified basic domain sequence may have at least 60%, 65%, 70%, 75%, 80%, 85%, 90%, or 95% identity with the basic domain of the second bZIP protein. The modified basic domain sequence may comprise 5, 6, 7, 8, 9, 10, 11, 12, 13, 14, 15, 16, 17, 18, 19, 20, 21, 22, 23, 24, or 25 residues from the basic domain of the second bZIP protein. In some aspects, the modified leucine zipper domain sequence of the second bZIP protein is at most, at least, or exactly 25, 24, 23, 22, 21, 20, 19, 18, 17, 16, 15, 14, 13, 12, 11, 10, 9, or 8 residues in length. In some aspects, the modified leucine zipper domain sequence of the second bZIP protein is at most 15 residues in length. In some aspects, the modified leucine zipper domain sequence of the second bZIP protein is 12 residues in length. The modified leucine zipper binding domain sequence may have at least 40%, 45%, 50%, 55%, 60%, 65%, 70%, 75%, 80%, 85%, 90%, or 95% identity with the leucine zipper domain of the second bZIP protein. The modified leucine zipper domain sequence may comprise 5, 6, 7, 8, 9, 10, 11, 12, 13, 14, 15, 16, 17, 18, 19, 20, 21, 22, 23, 24, or 25 residues from the leucine zipper domain of the second bZIP protein.

In some aspects, the first engineered peptide is at most 40 residues in length. In some aspects, the first engineered peptide is 32 residues in length. In some aspects, the second engineered peptide is at most 40 residues in length. In some aspects, the second engineered peptide is 32 residues in length. In some aspects, the modified basic domain sequence of the first bZIP protein comprises a serine substituted for any cysteine relative to a native basic domain sequence of the first bZIP protein. In some aspects, the modified basic domain sequence of the second bZIP protein comprises a serine substituted for any cysteine relative to a native basic domain sequence of the second bZIP protein. In some aspects, the modified leucine zipper domain sequence of the first bZIP protein comprises an alanine substituted for any cysteine at a “b”, “c”, or “f” position relative to a native leucine zipper domain sequence of the first bZIP protein, the modified leucine zipper domain sequence of the first bZIP protein comprises a leucine substituted for any cysteine at an “a” or “d” position relative to a native leucine zipper domain sequence of the first bZIP protein. In some aspects, the modified leucine zipper domain sequence of the second bZIP protein comprises an alanine substituted for any cysteine at a “b”, “c”, or “f” position relative to a native leucine zipper domain sequence of the second bZIP protein. In some aspects, the modified leucine zipper domain sequence of the second bZIP protein comprises a leucine substituted for any cysteine at an “a” or “d” position relative to a native leucine zipper domain sequence of the second bZIP protein.

In some aspects, the modified basic domain sequence of the first bZIP protein comprises a cysteine at a position corresponding to the last position of a native basic domain sequence of the first bZIP protein. In some aspects, the modified leucine zipper domain sequence of the second bZIP protein comprises a lysine at a position corresponding to a first “e” position of a native leucine zipper domain sequence of the second bZIP protein. In some aspects, the interpeptide linkage is between the cysteine and the lysine. In some aspects, the modified leucine zipper domain sequence of the first bZIP protein comprises a leucine in place of any residue at an “a” or “d” position that is not a leucine or isoleucine relative to a native leucine zipper domain of the first bZIP protein. In some aspects, the modified leucine zipper domain sequence of the second bZIP protein comprises a leucine in place of any residue at an “a” or “d” position that is not a leucine or isoleucine relative to a native leucine zipper domain of the second bZIP protein.

In some aspects, the first bZIP protein is c-Fos. In some aspects, the modified DNA-binding domain sequence of c-Fos comprises: IRRERNKMAAAKSRNRRREC (SEQ ID NO:16); IRR#RNK#AAAKSRNRRREC (SEQ ID NO:17); EEKRRIRRERNKMAAAKSRNRRREC (SEQ ID NO:18); or EEKRRIRR#RNK#AAAKSRNRRREC (SEQ ID NO:19); wherein # are intrapeptide stabilizing linkage sites which together form the structure

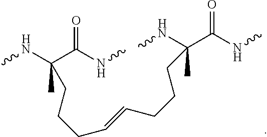

In some aspects, the modified leucine zipper domain sequence of c-Fos comprises: TDTLEDETDQLE (SEQ ID NO20); LDELQAEIEQLE (SEQ ID NO:21); IDELQAEIEQLE (SEQ ID NO:22); IDEIQAEIEQIE (SEQ ID NO:23); L#ELQ#EIEQLE (SEQ ID NO:24); I#ELQ#EIEQLE (SEQ ID NO:25); or I#EIQ#EIEQIE (SEQ ID NO:26); wherein # are intrapeptide stabilizing linkage sites, which together form the structure

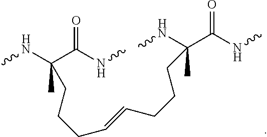

In some aspects, the second bZIP protein is c-Jun. In some aspects, the modified DNA-binding domain sequence of c-Jun is: RKRMRNRIAASKSRKRKLER (SEQ ID NO:27); RKR#RNR#AASKSRKRKLER (SEQ ID NO:28); RIKAERKRMRNRIAASKSRKRKLER (SEQ ID NO:29); or RIKAERKR#RNR#AASKSRKRKLER (SEQ ID NO:30); wherein # are intrapeptide stabilizing linkage sites, which together form the structure

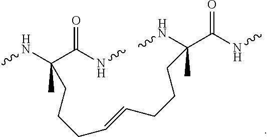

In some aspects, the modified leucine zipper domain sequence of c-Jun comprises: IAKmLEEKVKTLK (SEQ ID NO:31); IARLKmEKVKTLK (SEQ ID NO:32); AAELKmEKVATLK (SEQ ID NO:33); IARLKmEKIKTLK (SEQ ID NO:34); IARIKmEKIKTIK (SEQ ID NO:35); I#RLKm#KVKTLK (SEQ ID NO:36); or I#RLKm#KIKTLK (SEQ ID NO:37); wherein # are intrapeptide stabilizing linkage sites, which together form the structure

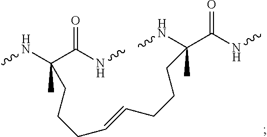

and Km is a Lys residue attached to a maleimide-linker forming a portion of the structure:

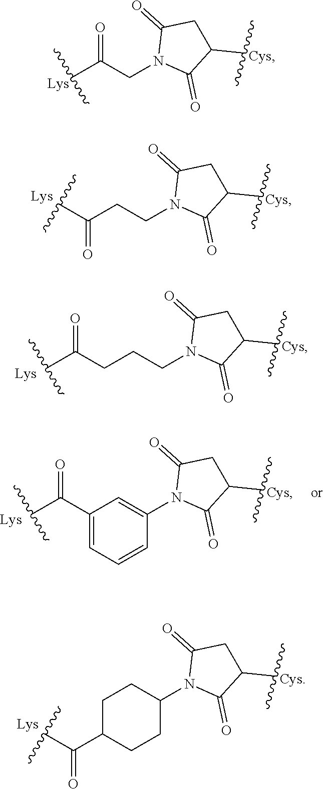 [SMILES: *C(=O)CN1C(=O)CC(*)C1=O ; *C(=O)CCN1C(=O)CC(*)C1=O ; *C(=O)CCCN1C(=O)CC(*)C1=O ; *C(=O)c1cccc(N2C(=O)CC(*)C2=O)c1 ; *C(=O)C1CCC(N2C(=O)CC(*)C2=O)CC1]

In some aspects, the first bZIP protein is XBP1. In some aspects, the modified DNA-binding domain sequence of XBP1 comprises: RRKLKNRVAAQTARDRKKAC (SEQ ID NO:38); or RRK#KNR#AAQTARDRKKAC (SEQ ID NO:39); wherein # are intrapeptide stabilizing linkage sites, which together form the structure

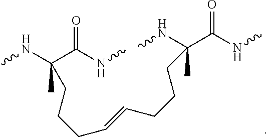

In some aspects, the modified leucine zipper domain sequence of XBP1 comprises: MSELEQQVVDLE (SEQ ID NO:40); LSELEQQVVDLE (SEQ ID NO:41); or L#ELE #QVVDLE (SEQ ID NO:42); wherein # are intrapeptide stabilizing linkage sites, which together form the structure

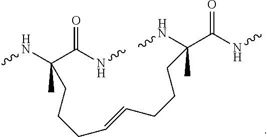

In some aspects, the second bZIP protein is XBP1. In some aspects, the modified DNA-binding domain sequence of XBP1 comprises: RRKLKNRVAAQTARDRKKAR (SEQ ID NO:43); or RRK#KNR#AAQTARDRKKAR (SEQ ID NO:44); wherein # are intrapeptide stabilizing linkage sites, which together form the structure

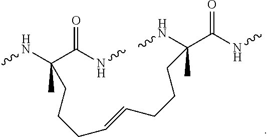

In some aspects, the modified leucine zipper domain sequence of XBP1 comprises: MSELKmQQVVDLE (SEQ ID NO:45); LSELKmQQVVDLE (SEQ ID NO:46); or L#ELKm#QVVDLE (SEQ ID NO:47); wherein # are intrapeptide stabilizing linkage sites, which together form the structure

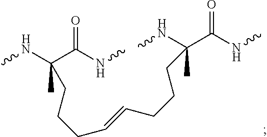

and Km is a Lys residue attached to a maleimide-linker forming a portion of the structure:

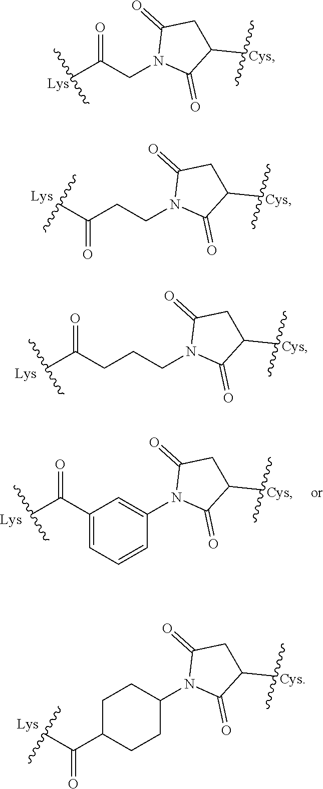 [SMILES: *C(=O)CN1C(=O)CC(*)C1=O ; *C(=O)CCN1C(=O)CC(*)C1=O ; *C(=O)CCCN1C(=O)CC(*)C1=O ; *C(=O)c1cccc(N2C(=O)CC(*)C2=O)c1 ; *C(=O)C1CCC(N2C(=O)CC(*)C2=O)CC1]

In some aspects, the first bZIP protein is ATF4. In some aspects, the modified DNA-binding domain sequence of ATF4 comprises: KKMEQNKTAATRYRQKKRAC (SEQ ID NO:48); wherein # are intrapeptide stabilizing linkage sites, which together form the structure

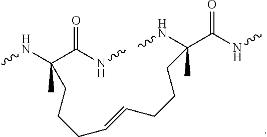

In some aspects, the modified leucine zipper domain sequence of ATF4 comprises: QEALTGELKELE (SEQ ID NO:49); LEALKAELKELR (SEQ ID NO:50); or L#ALK#ELKELR (SEQ ID NO:51).

In some aspects, the first bZIP protein is C/EBPO. In some aspects, the modified DNA-binding domain sequence of C/EBPβ comprises IRRERNNIAVRKSRDKAKMC (SEQ ID NO:52); wherein # are intrapeptide stabilizing linkage sites, which together form the structure

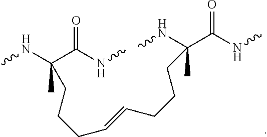

In some aspects, the modified leucine zipper domain sequence of C/EBPO comprises: LLELQHKVLELR (SEQ ID NO:53); or L#ELQ#KVLELR (SEQ ID NO:54); wherein # are intrapeptide stabilizing linkage sites, which together form the structure

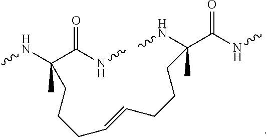

In some aspects, the second bZIP protein is ATF4. In some aspects, the modified DNA-binding domain sequence of ATF4 comprises KKMEQNKTAATRYRQKKRAE (SEQ ID NO:55); wherein # are intrapeptide stabilizing linkage sites, which together form the structure

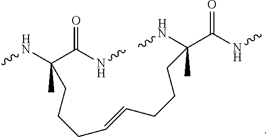

In some aspects, the modified leucine zipper domain sequence of ATF4 comprises: QEALKmGELKELE (SEQ ID NO:56); LEALKmAELKELR (SEQ ID NO:57); or L#ALKm#ELKELR (SEQ ID NO:58); wherein # are intrapeptide stabilizing linkage sites, which together form the structure

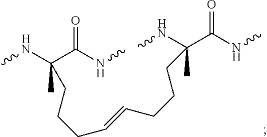

and Km is a Lys residue attached to a maleimide-linker forming a portion of the structure:

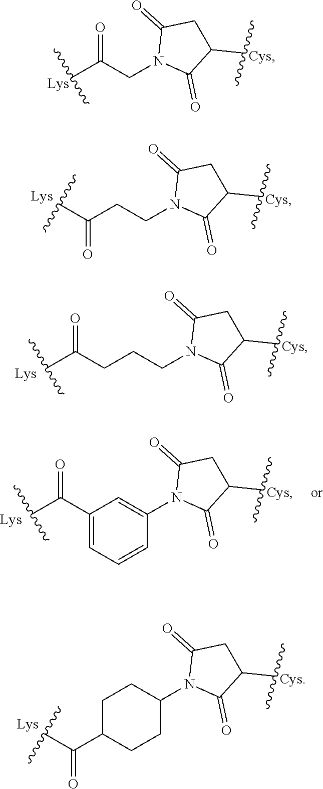 [SMILES: *C(=O)CN1C(=O)CC(*)C1=O ; *C(=O)CCN1C(=O)CC(*)C1=O ; *C(=O)CCCN1C(=O)CC(*)C1=O ; *C(=O)c1cccc(N2C(=O)CC(*)C2=O)c1 ; *C(=O)C1CCC(N2C(=O)CC(*)C2=O)CC1]

In some aspects, the first engineered peptide comprises a intrapeptide stabilizing linkage. In some aspects, the second engineered peptide comprises a intrapeptide stabilizing linkage. In some aspects, the intrapeptide stabilizing linkage is between the fourth position and the eighth position of the first engineered peptide. In some aspects, the intrapeptide stabilizing linkage is between the twenty-second position and the twenty-sixth position of the first engineered peptide. In some aspects, the interpeptide linkage comprises a maleimide-thiol adduct. In some aspects, the interpeptide linkage is

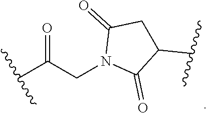 [SMILES: *C(=O)CN1C(=O)CC(*)C1=O]

Disclosed herein, in certain aspects, is an engineered DNA-binding dimer having one of the following formulas:

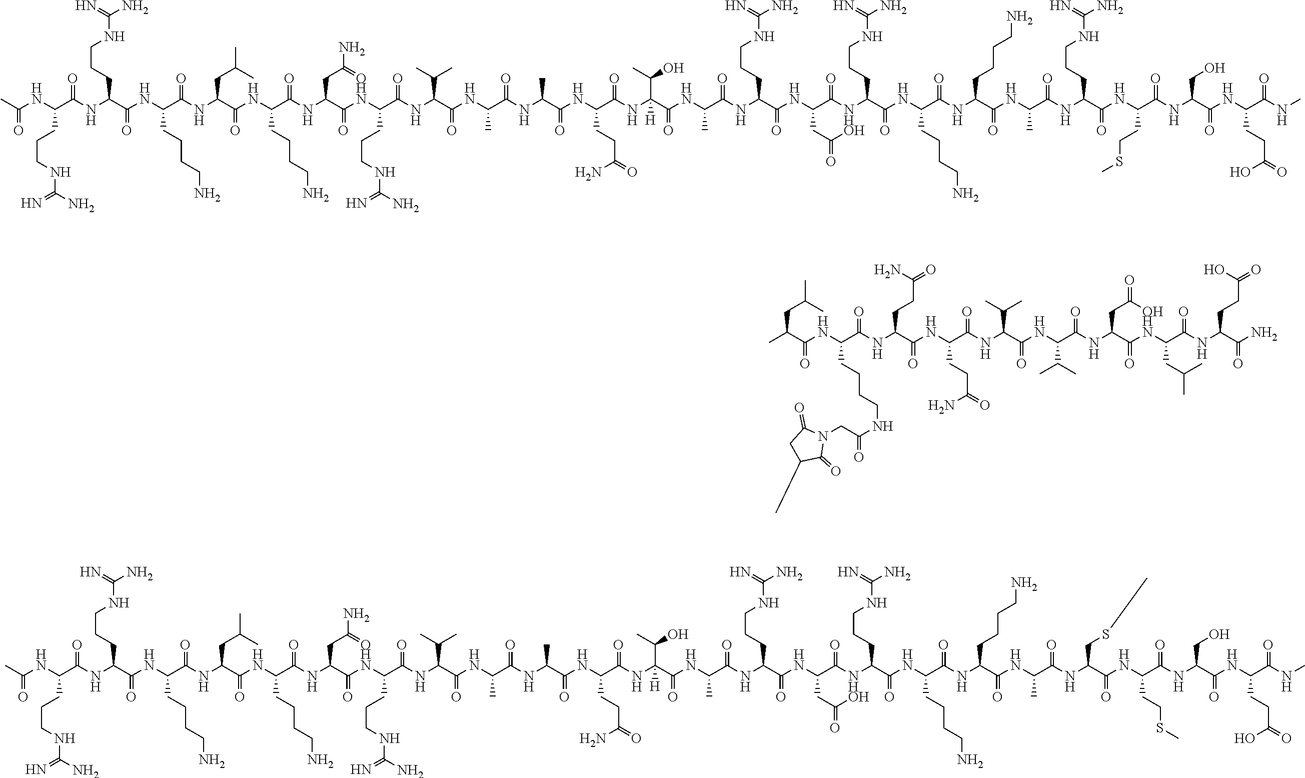

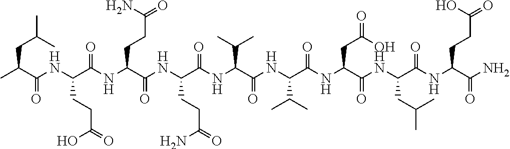 [SMILES: CC(C)C[C@H](NC(=O)[C@H](CC(=O)O)NC(=O)[C@@H](NC(=O)[C@@H](NC(=O)[C@H](CCC(N)=O)NC(=O)[C@H](CCC(N)=O)NC(=O)[C@H](CCC(=O)O)NC(=O)[C@@H](C)CC(C)C)C(C)C)C(C)C)C(=O)N[C@@H](CCC(=O)O)C(N)=O]

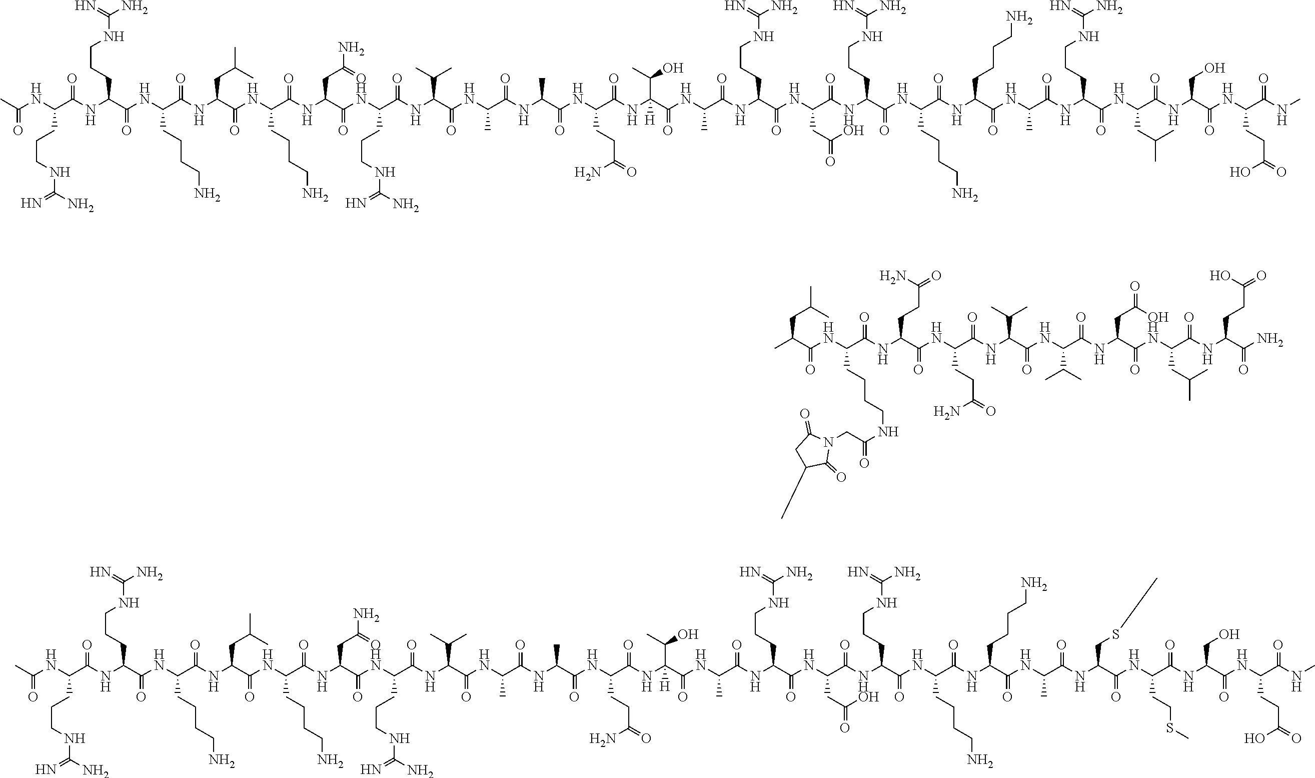

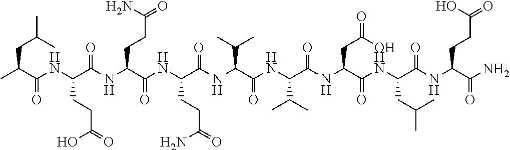 [SMILES: CC(C)C[C@H](NC(=O)[C@H](CC(=O)O)NC(=O)[C@@H](NC(=O)[C@@H](NC(=O)[C@H](CCC(N)=O)NC(=O)[C@H](CCC(N)=O)NC(=O)[C@H](CCC(=O)O)NC(=O)[C@@H](C)CC(C)C)C(C)C)C(C)C)C(=O)N[C@@H](CCC(=O)O)C(N)=O]

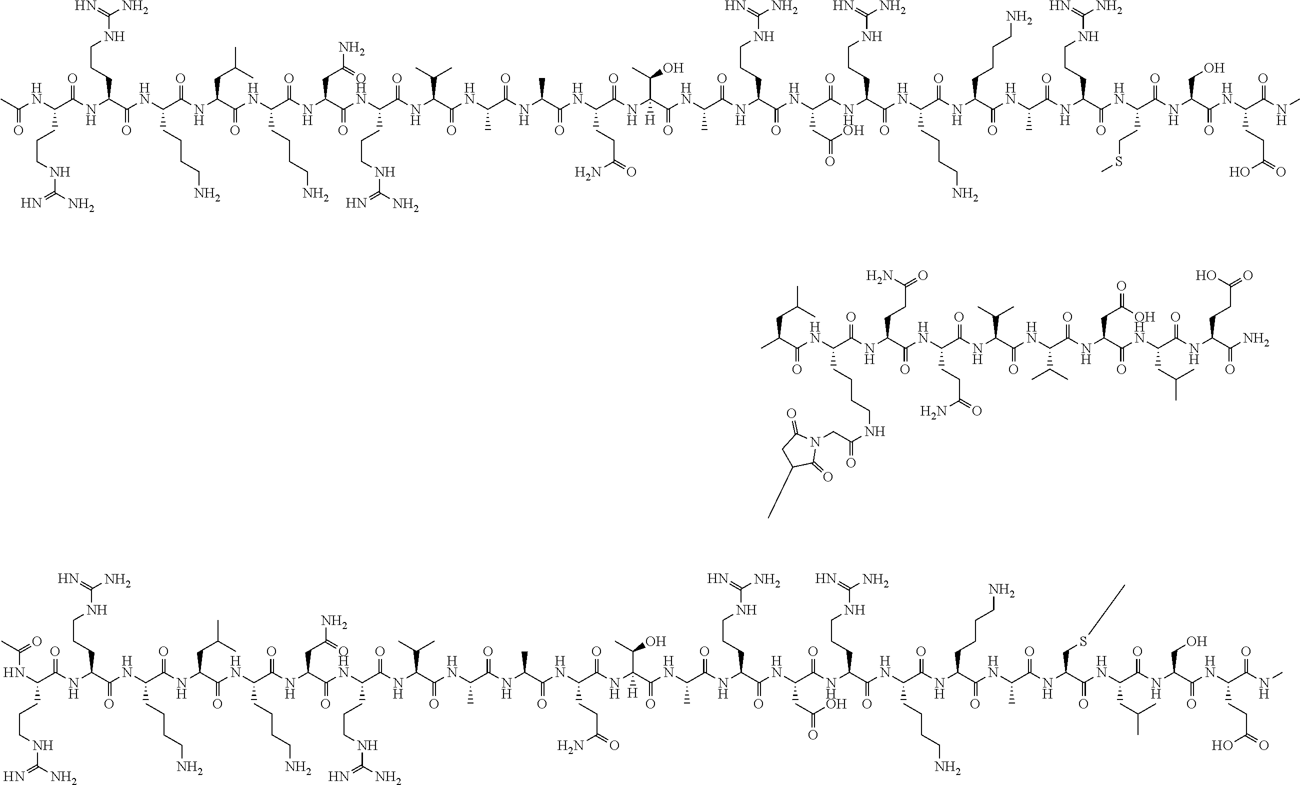

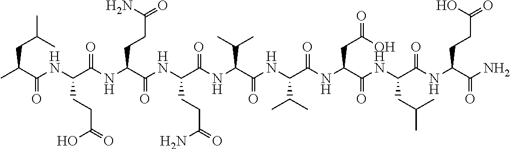 [SMILES: CC(C)C[C@H](NC(=O)[C@H](CC(=O)O)NC(=O)[C@@H](NC(=O)[C@@H](NC(=O)[C@H](CCC(N)=O)NC(=O)[C@H](CCC(N)=O)NC(=O)[C@H](CCC(=O)O)NC(=O)[C@@H](C)CC(C)C)C(C)C)C(C)C)C(=O)N[C@@H](CCC(=O)O)C(N)=O]

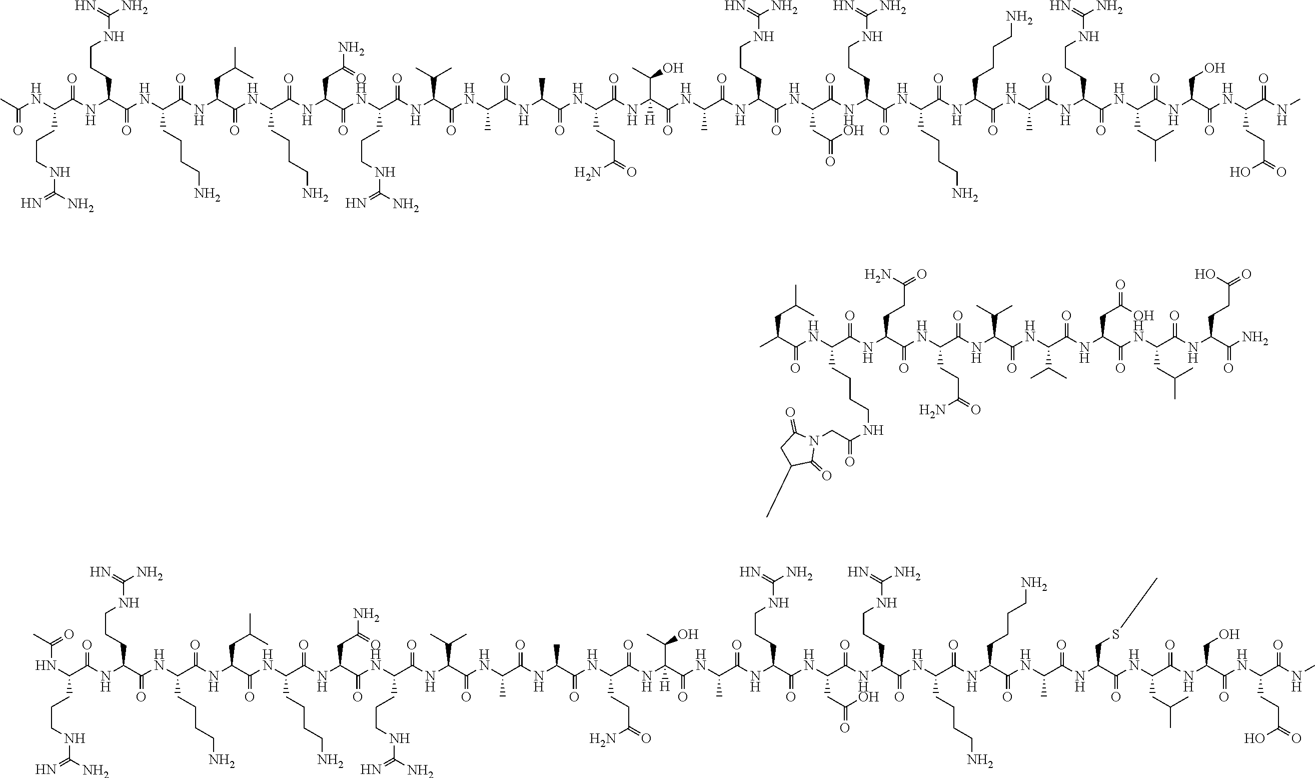

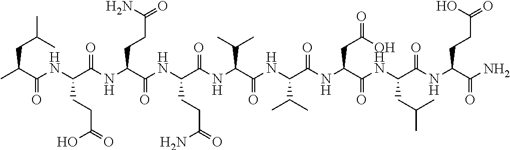 [SMILES: CC(C)C[C@H](NC(=O)[C@H](CC(=O)O)NC(=O)[C@@H](NC(=O)[C@@H](NC(=O)[C@H](CCC(N)=O)NC(=O)[C@H](CCC(N)=O)NC(=O)[C@H](CCC(=O)O)NC(=O)[C@@H](C)CC(C)C)C(C)C)C(C)C)C(=O)N[C@@H](CCC(=O)O)C(N)=O]

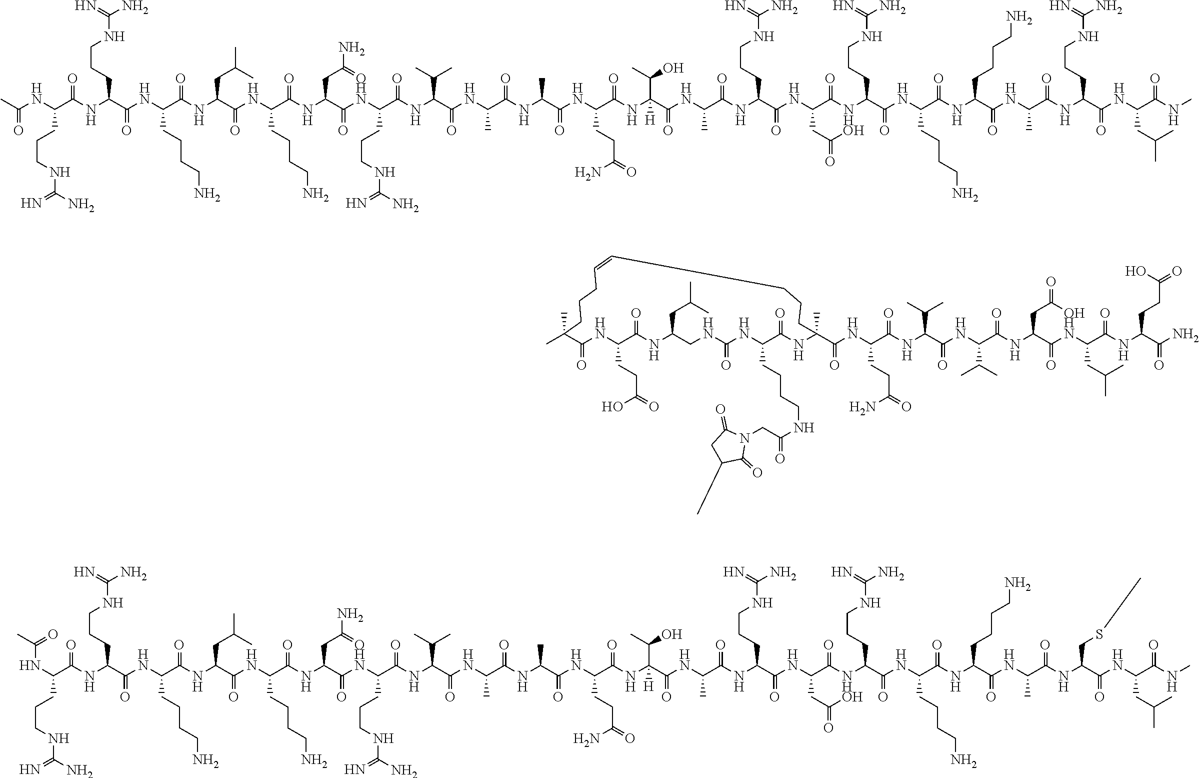

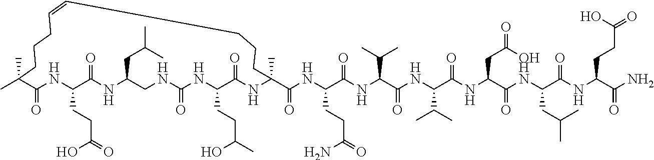

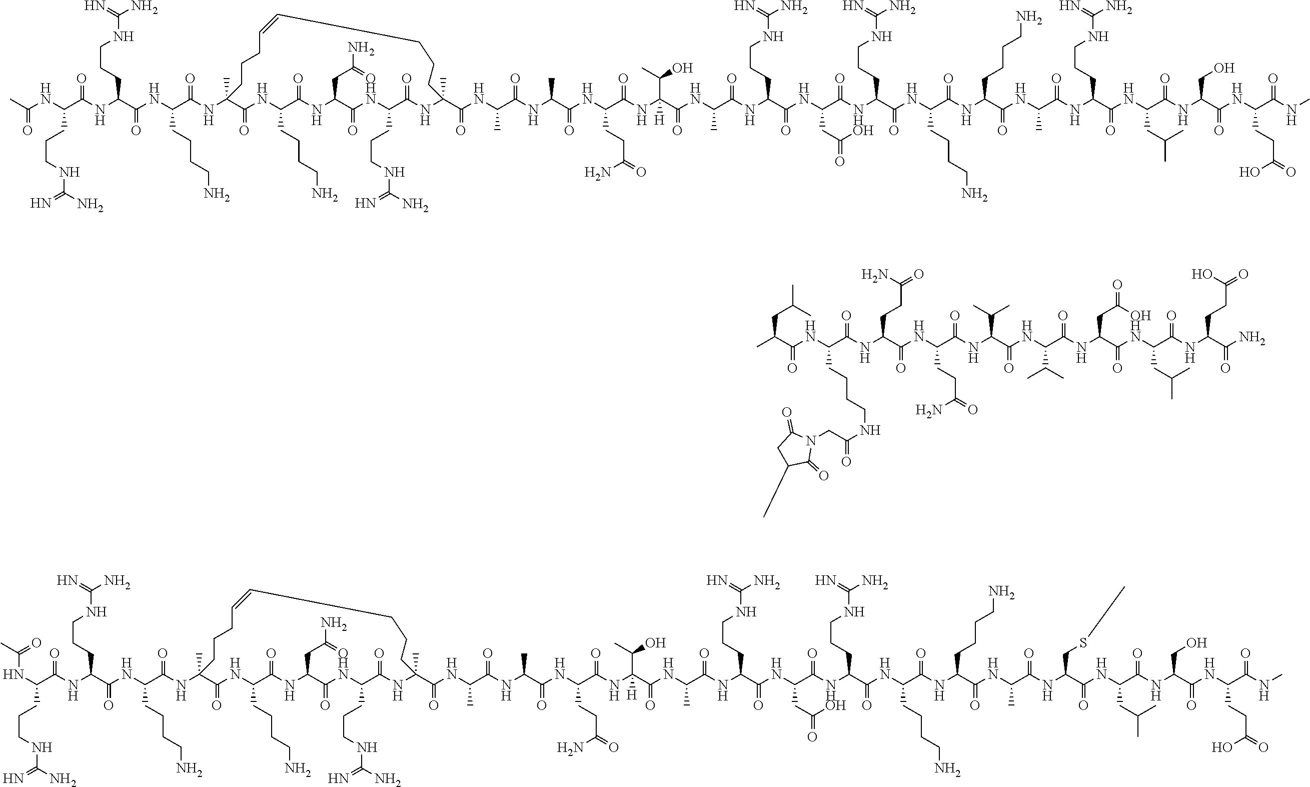

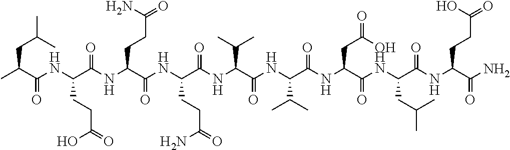 [SMILES: CC(C)C[C@H](NC(=O)[C@H](CC(=O)O)NC(=O)[C@@H](NC(=O)[C@@H](NC(=O)[C@H](CCC(N)=O)NC(=O)[C@H](CCC(N)=O)NC(=O)[C@H](CCC(=O)O)NC(=O)[C@@H](C)CC(C)C)C(C)C)C(C)C)C(=O)N[C@@H](CCC(=O)O)C(N)=O]

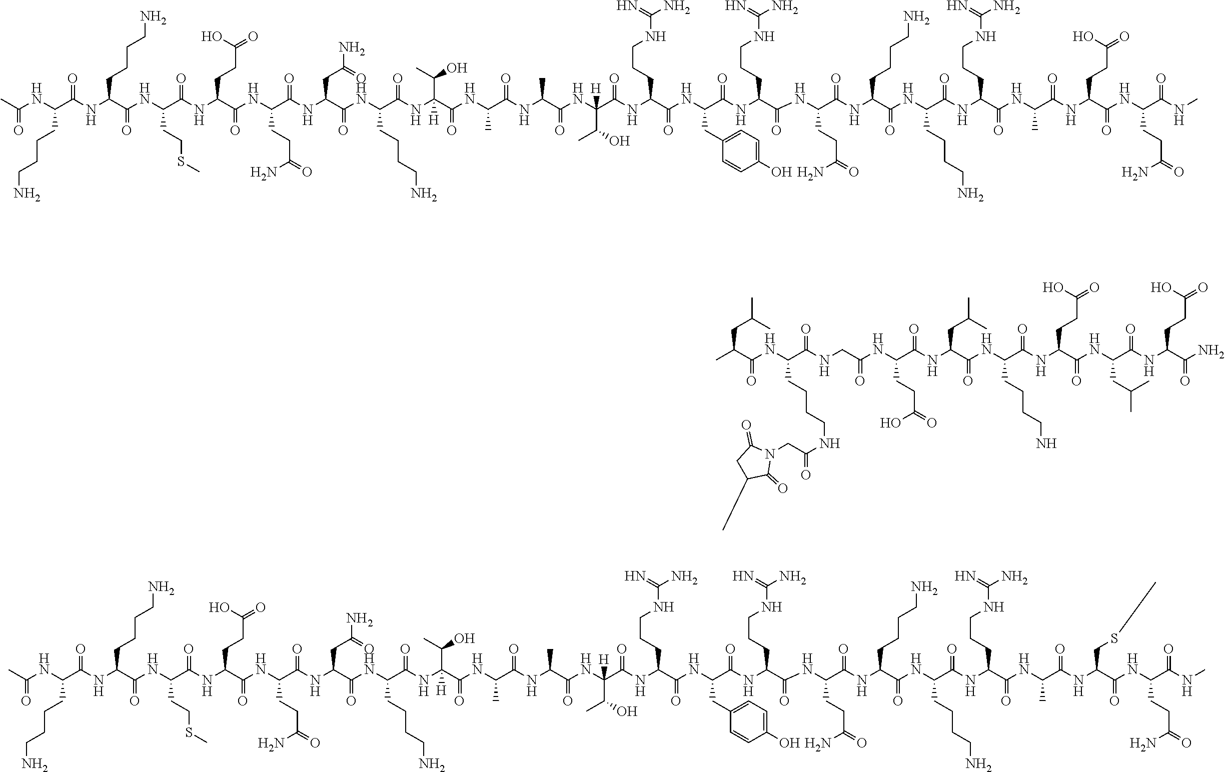

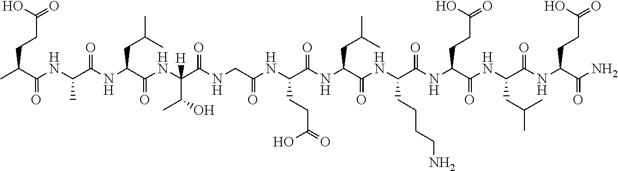 [SMILES: CC(C)C[C@H](NC(=O)[C@H](C)NC(=O)[C@@H](C)CCC(=O)O)C(=O)N[C@H](C(=O)NCC(=O)N[C@@H](CCC(=O)O)C(=O)N[C@@H](CC(C)C)C(=O)N[C@@H](CCCCN)C(=O)N[C@@H](CCC(=O)O)C(=O)N[C@@H](CC(C)C)C(=O)N[C@@H](CCC(=O)O)C(N)=O)[C@@H](C)O]

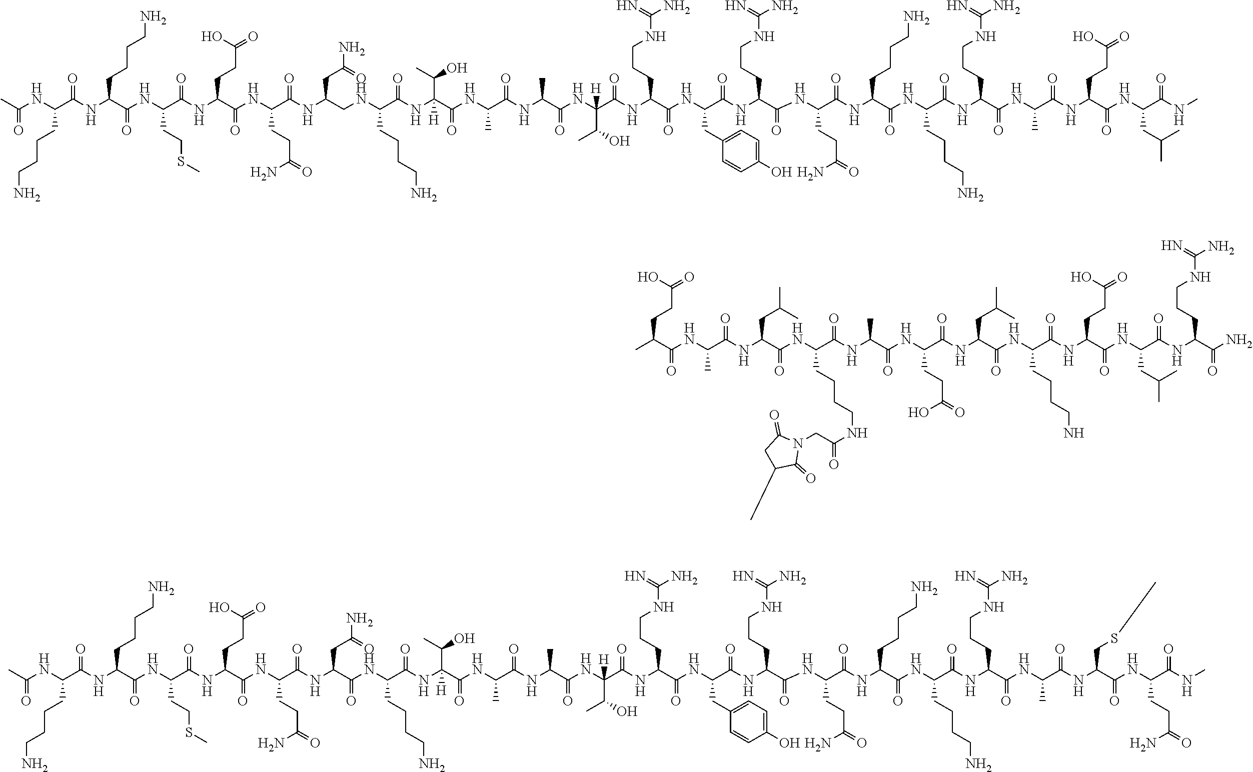

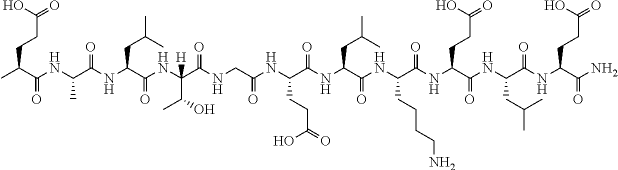 [SMILES: CC(C)C[C@H](NC(=O)[C@H](C)NC(=O)[C@@H](C)CCC(=O)O)C(=O)N[C@H](C(=O)NCC(=O)N[C@@H](CCC(=O)O)C(=O)N[C@@H](CC(C)C)C(=O)N[C@@H](CCCCN)C(=O)N[C@@H](CCC(=O)O)C(=O)N[C@@H](CC(C)C)C(=O)N[C@@H](CCC(=O)O)C(N)=O)[C@@H](C)O]

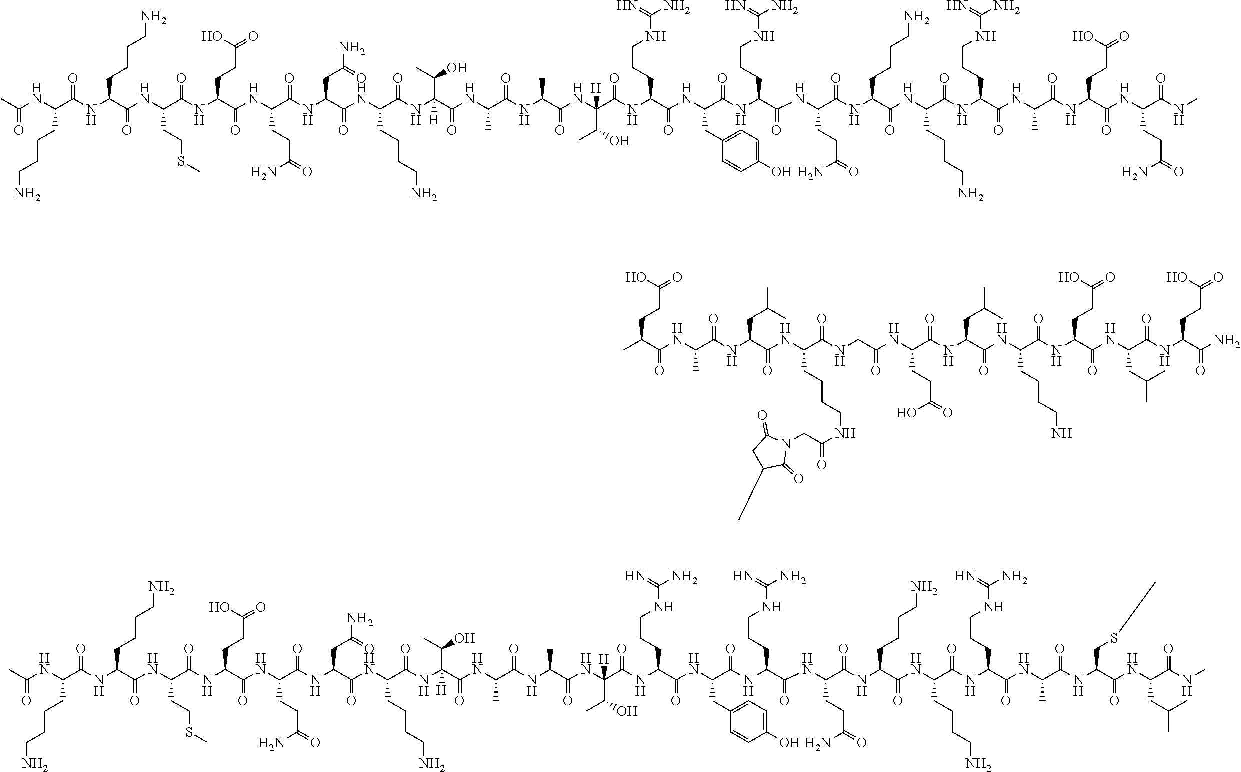

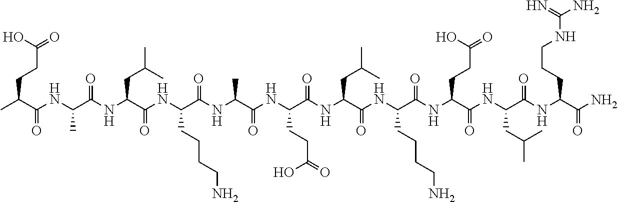 [SMILES: CC(C)C[C@H](NC(=O)[C@H](C)NC(=O)[C@@H](C)CCC(=O)O)C(=O)N[C@@H](CCCCN)C(=O)N[C@@H](C)C(=O)N[C@@H](CCC(=O)O)C(=O)N[C@@H](CC(C)C)C(=O)N[C@@H](CCCCN)C(=O)N[C@@H](CCC(=O)O)C(=O)N[C@@H](CC(C)C)C(=O)N[C@@H](CCCNC(=N)N)C(N)=O]

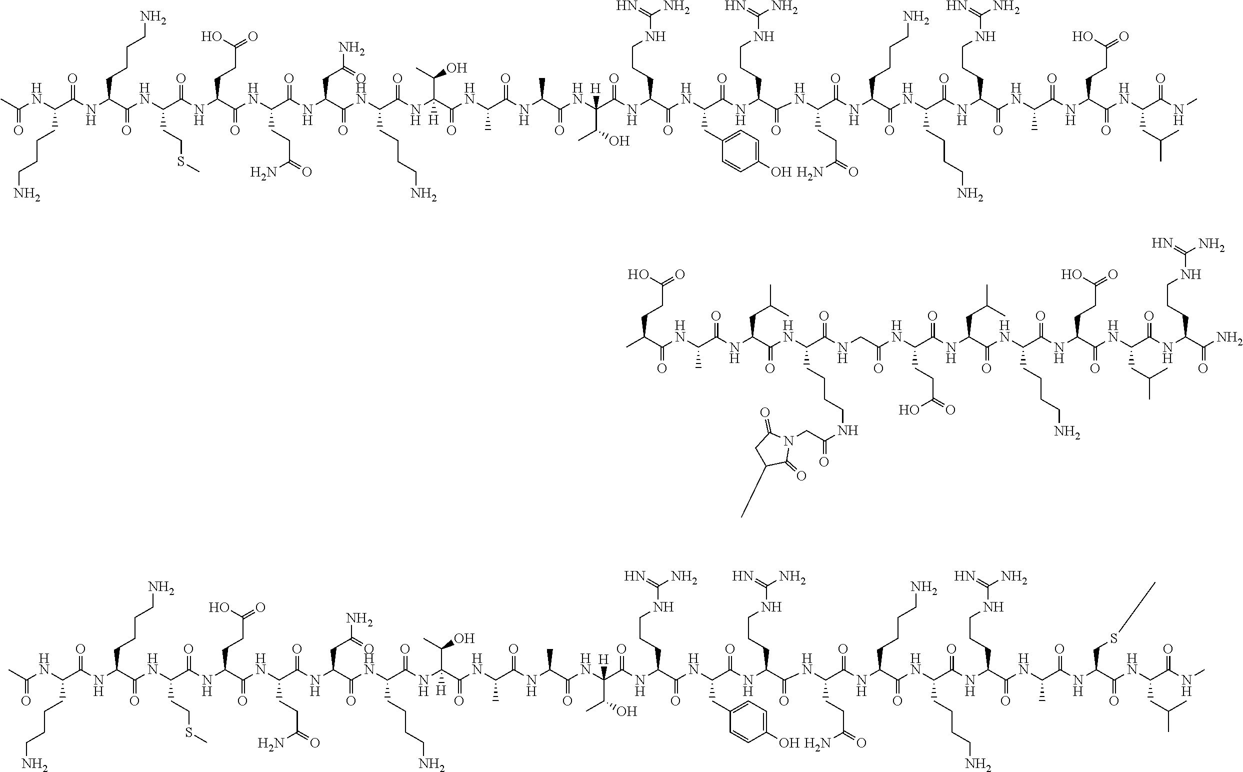

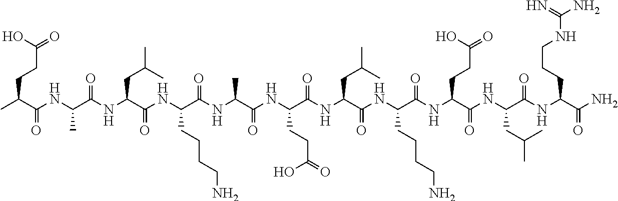 [SMILES: CC(C)C[C@H](NC(=O)[C@H](C)NC(=O)[C@@H](C)CCC(=O)O)C(=O)N[C@@H](CCCCN)C(=O)N[C@@H](C)C(=O)N[C@@H](CCC(=O)O)C(=O)N[C@@H](CC(C)C)C(=O)N[C@@H](CCCCN)C(=O)N[C@@H](CCC(=O)O)C(=O)N[C@@H](CC(C)C)C(=O)N[C@@H](CCCNC(=N)N)C(N)=O]

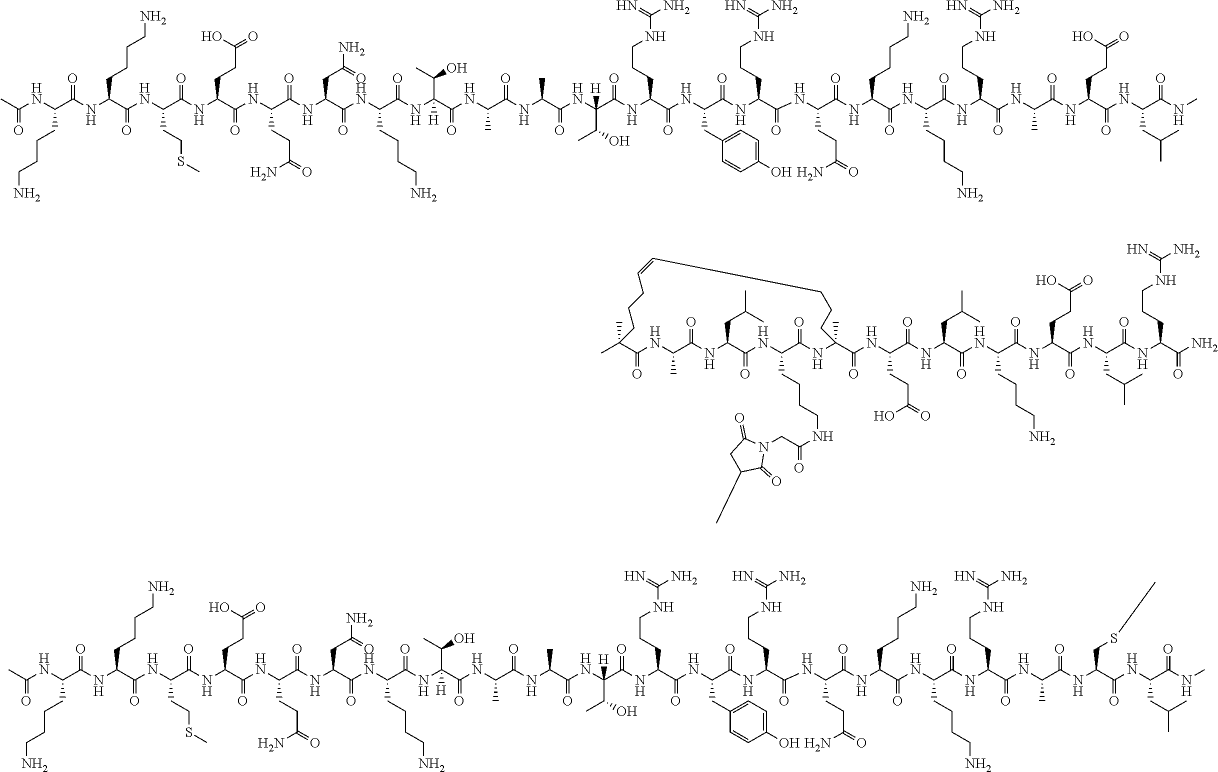

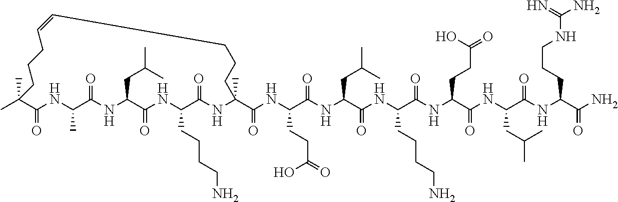

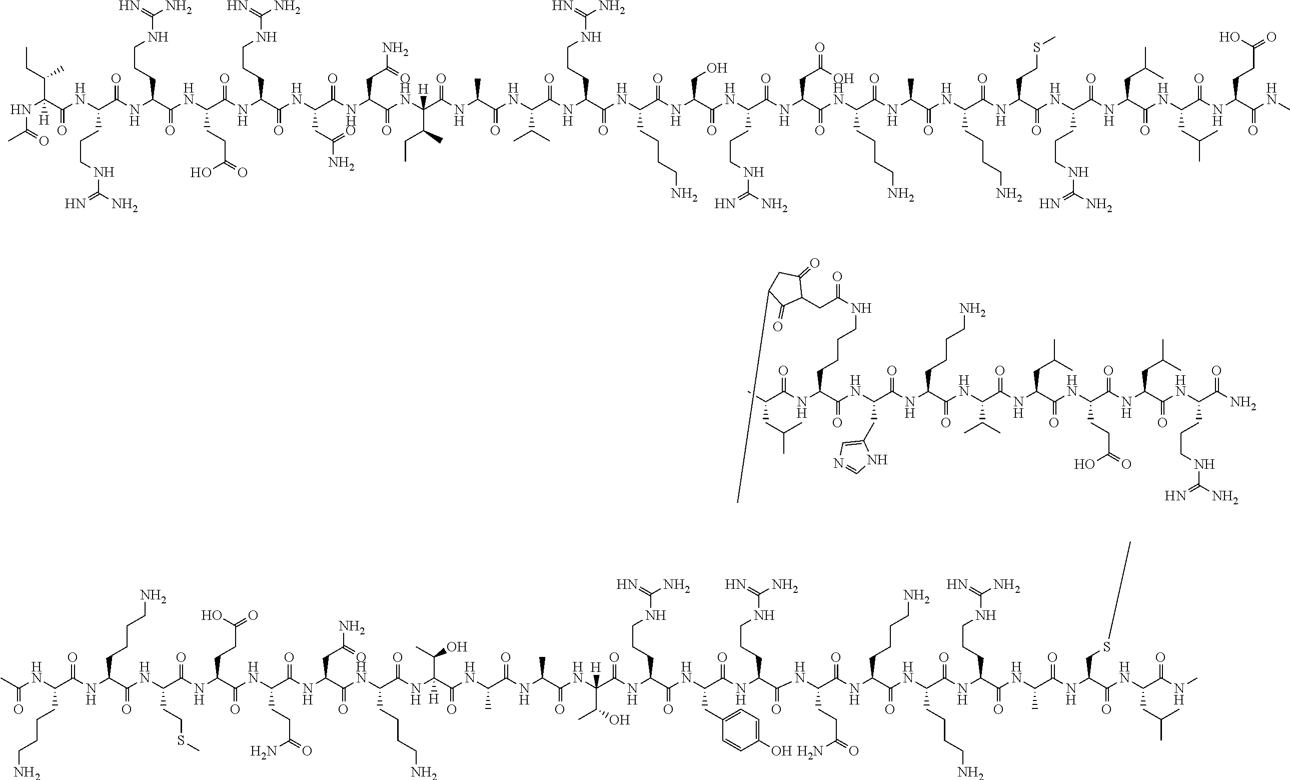

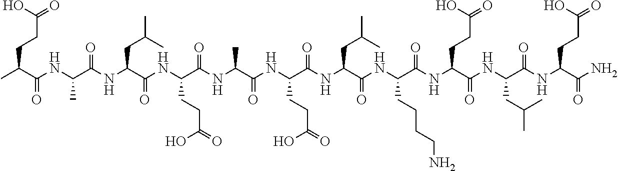 [SMILES: CC(C)C[C@H](NC(=O)[C@H](C)NC(=O)[C@@H](C)CCC(=O)O)C(=O)N[C@@H](CCC(=O)O)C(=O)N[C@@H](C)C(=O)N[C@@H](CCC(=O)O)C(=O)N[C@@H](CC(C)C)C(=O)N[C@@H](CCCCN)C(=O)N[C@@H](CCC(=O)O)C(=O)N[C@@H](CC(C)C)C(=O)N[C@@H](CCC(=O)O)C(N)=O]

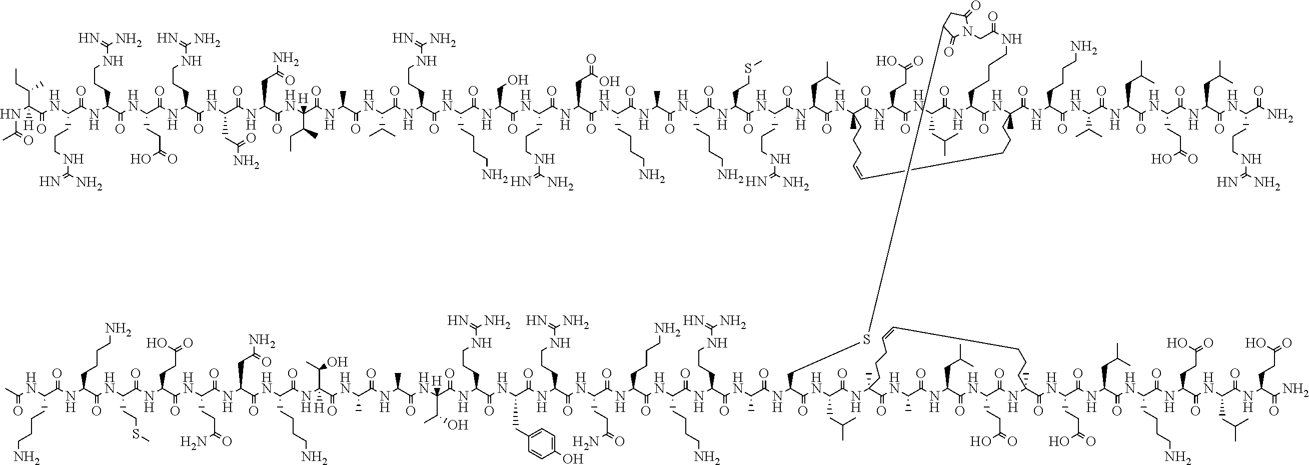

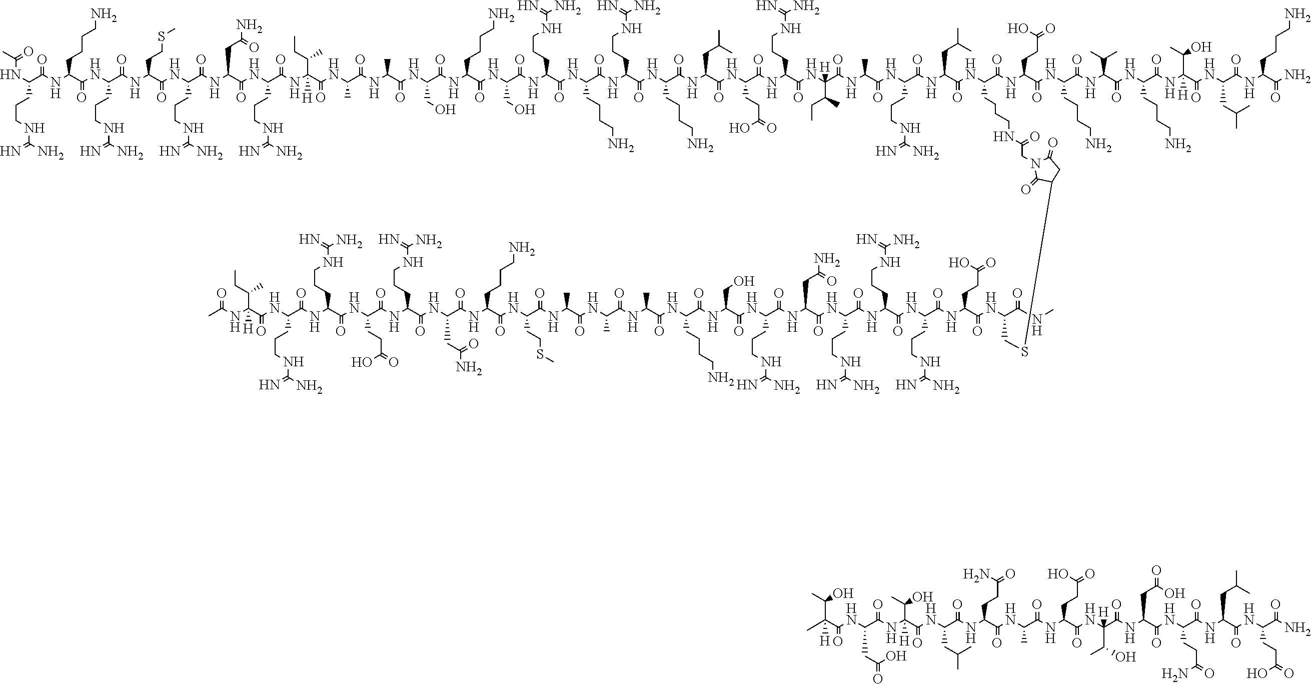

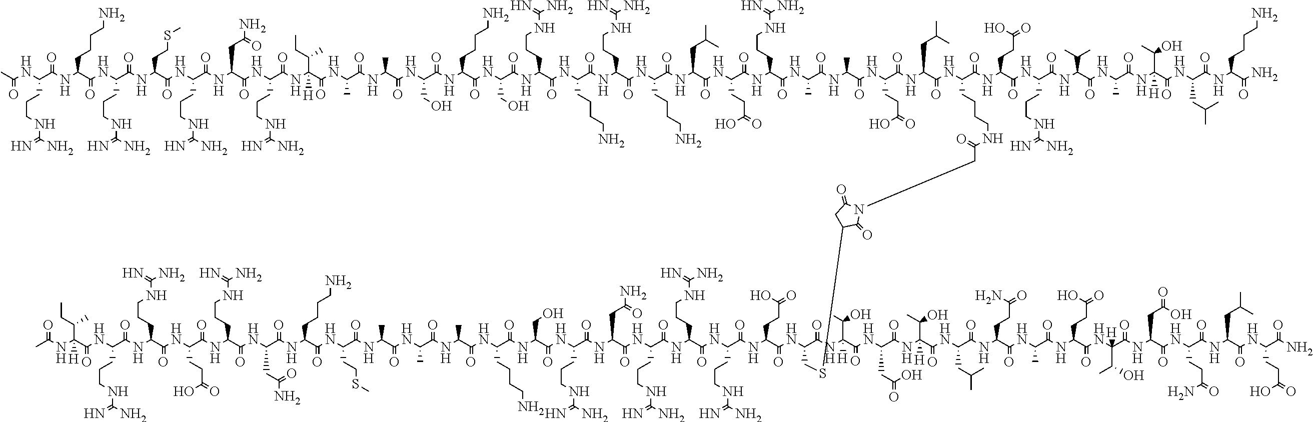

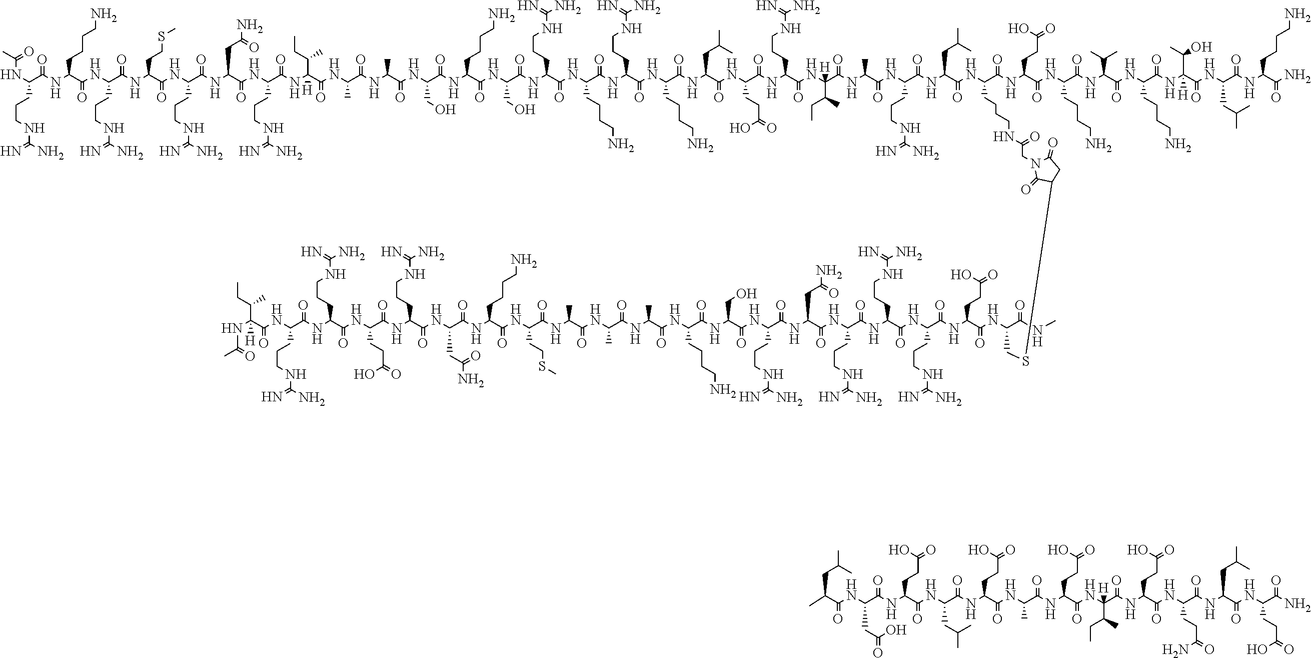

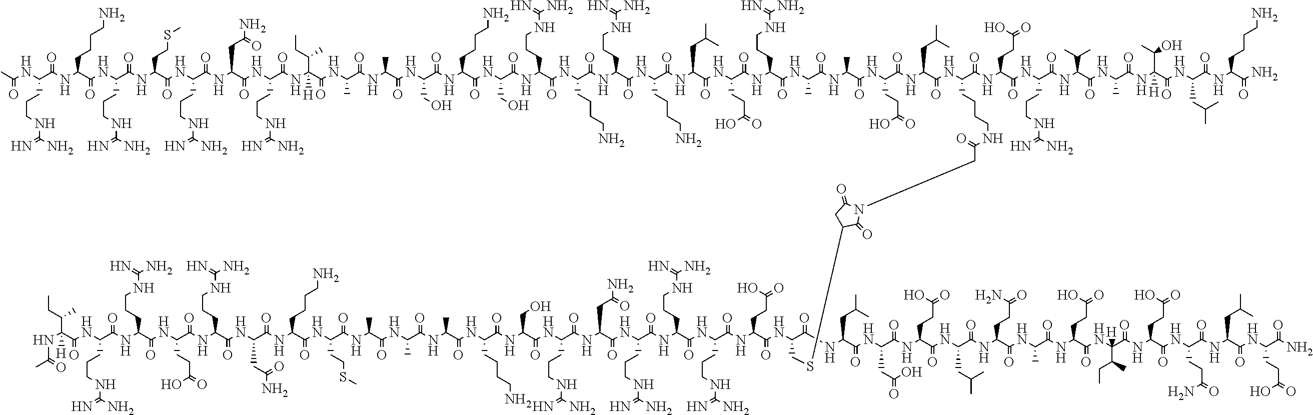

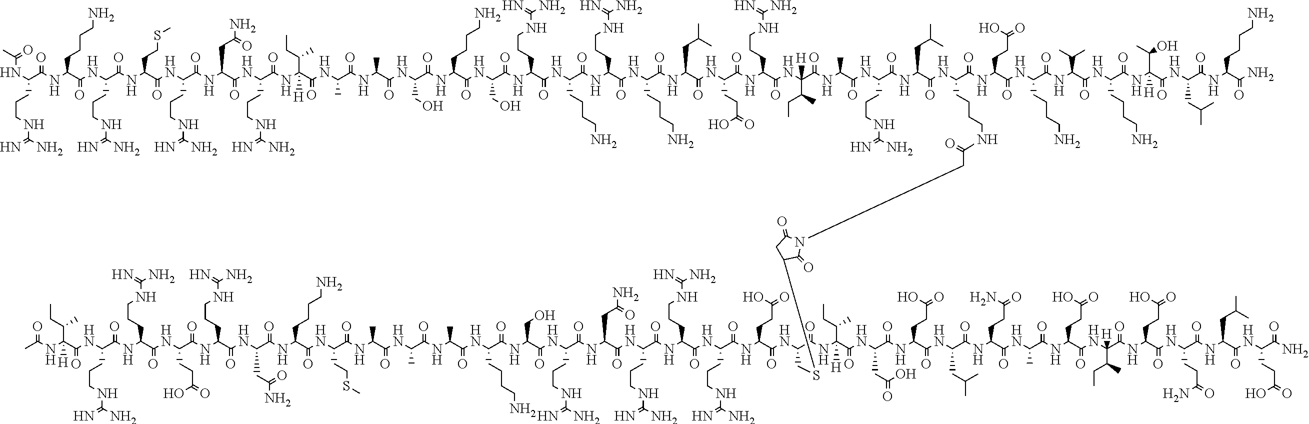

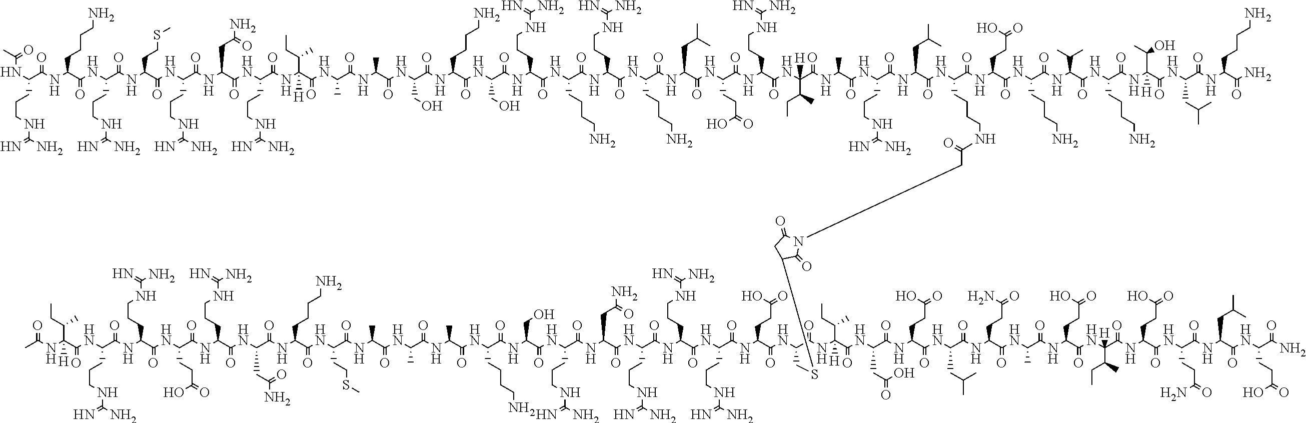

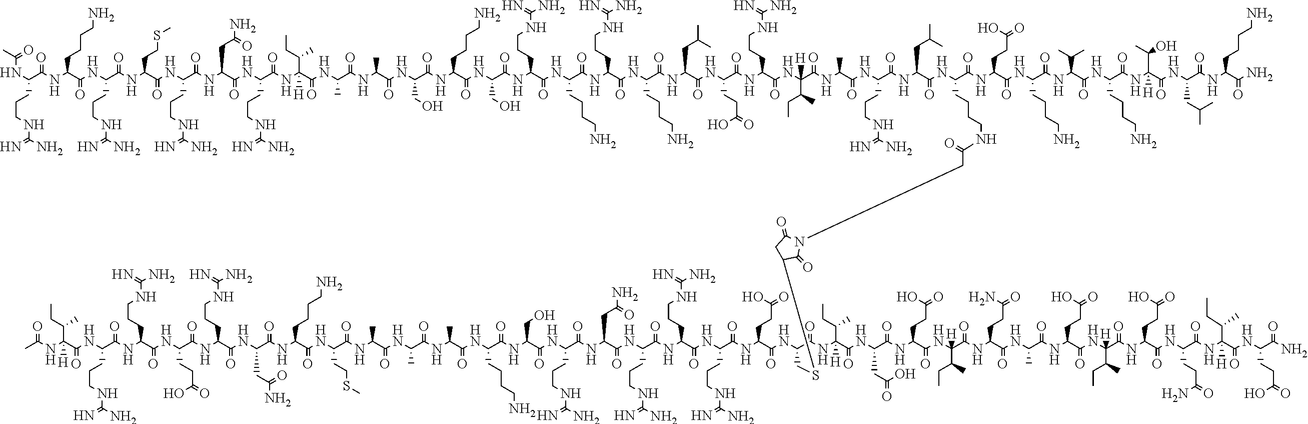

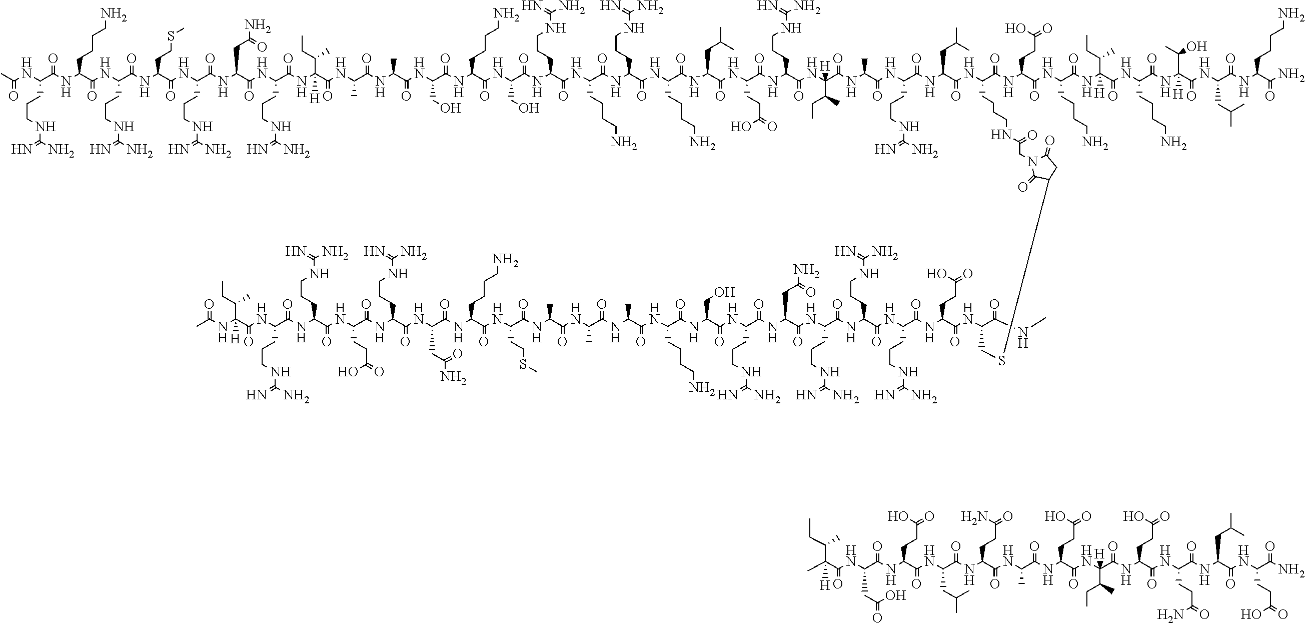

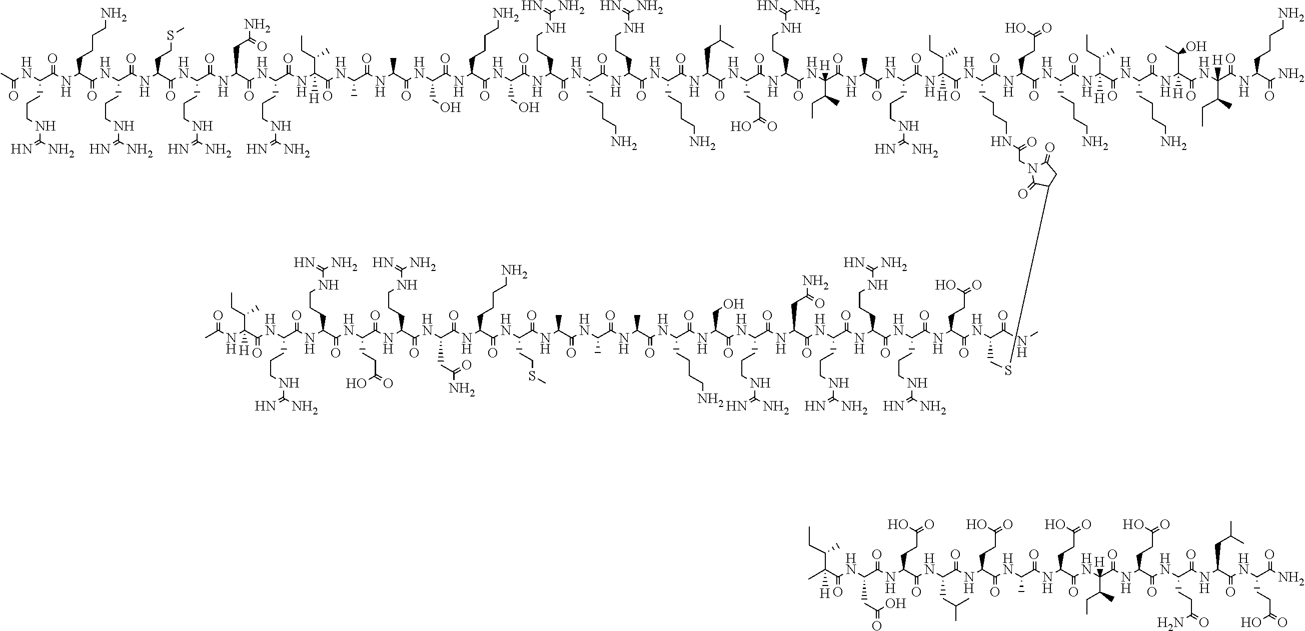

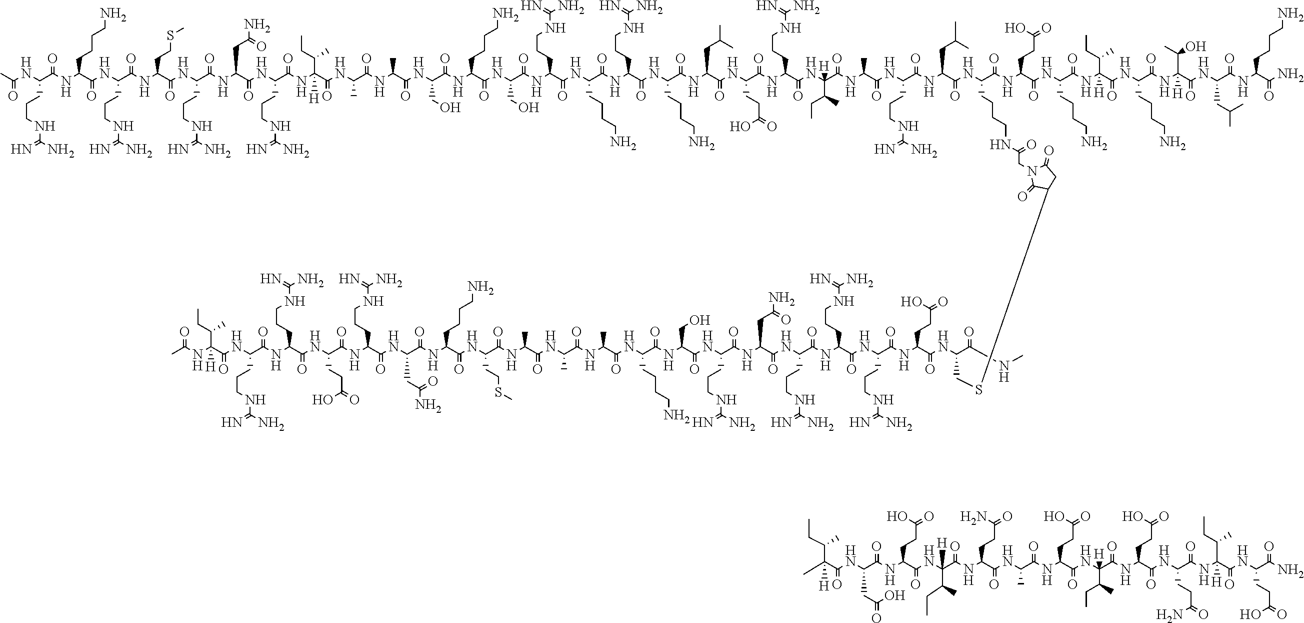

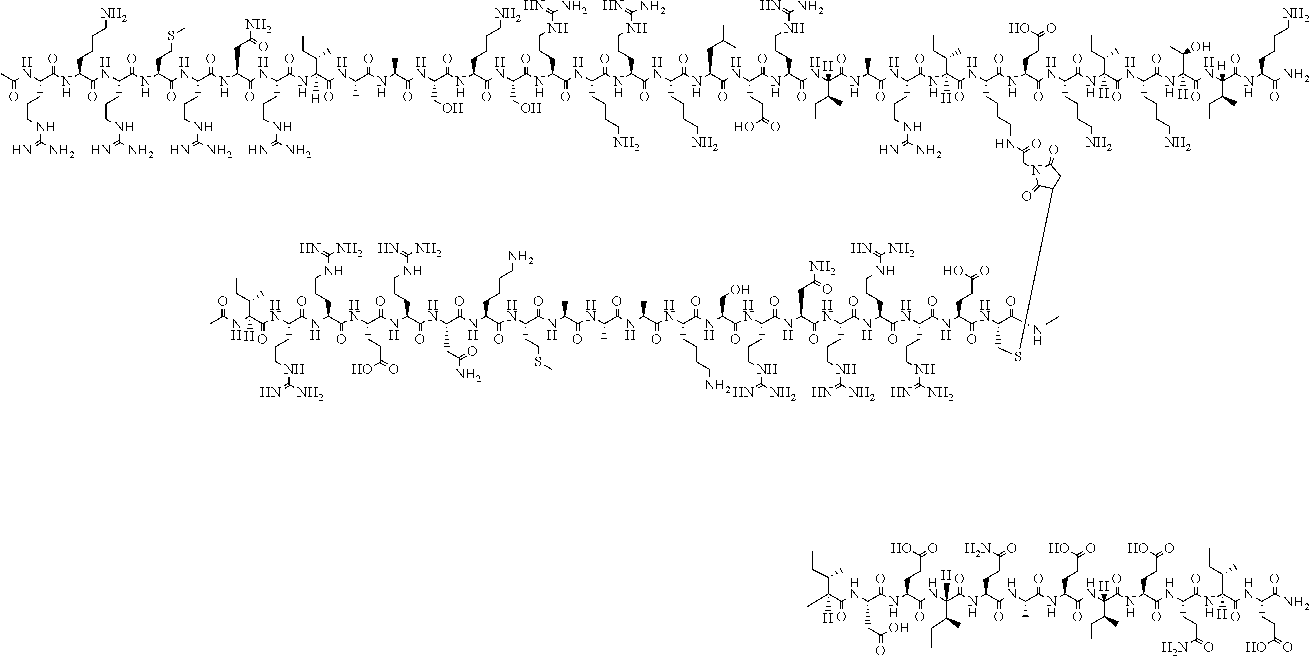

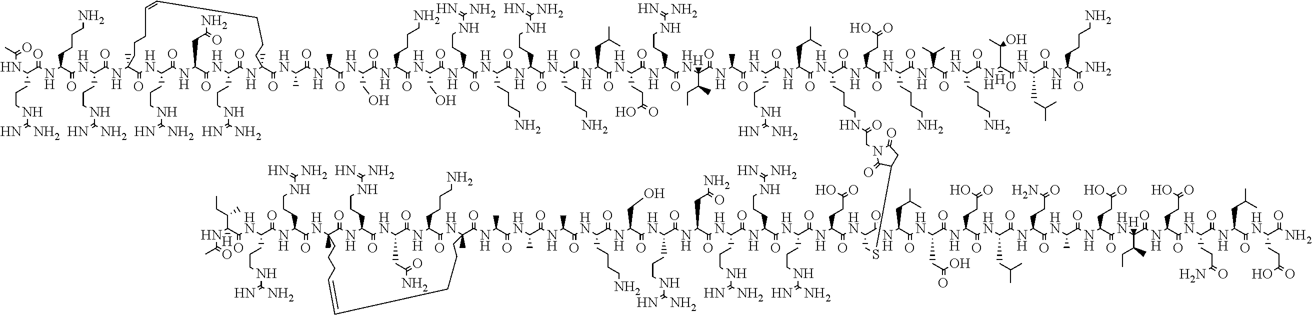

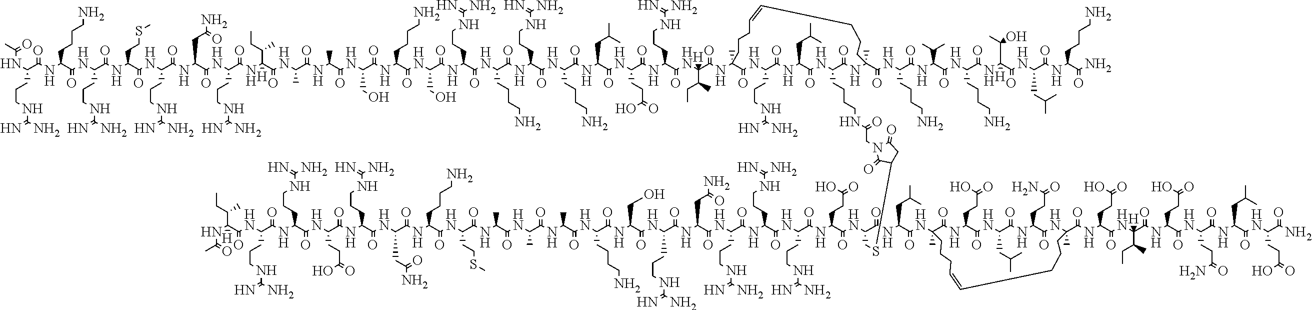

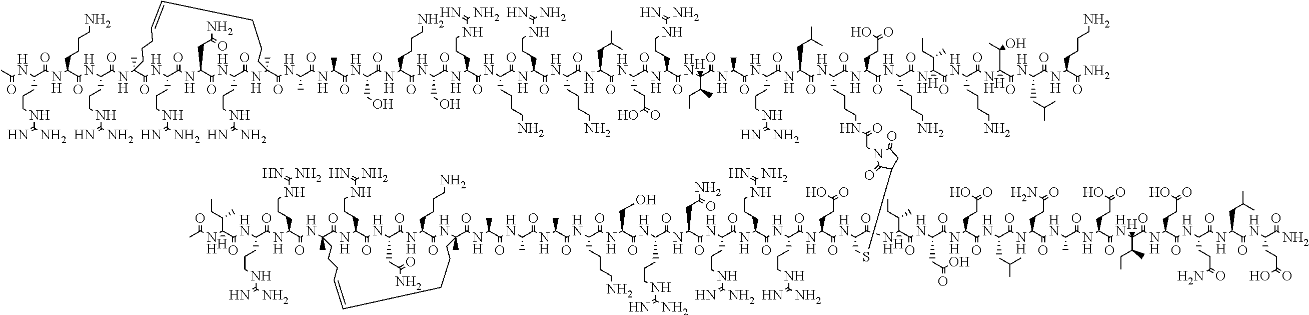

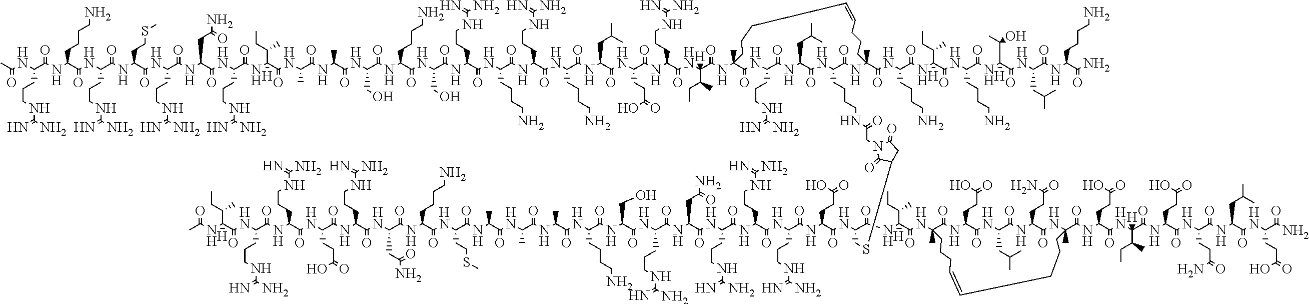

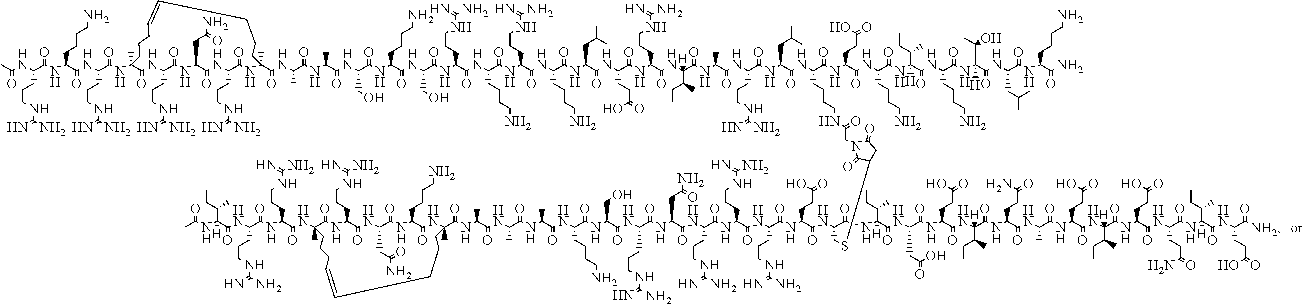

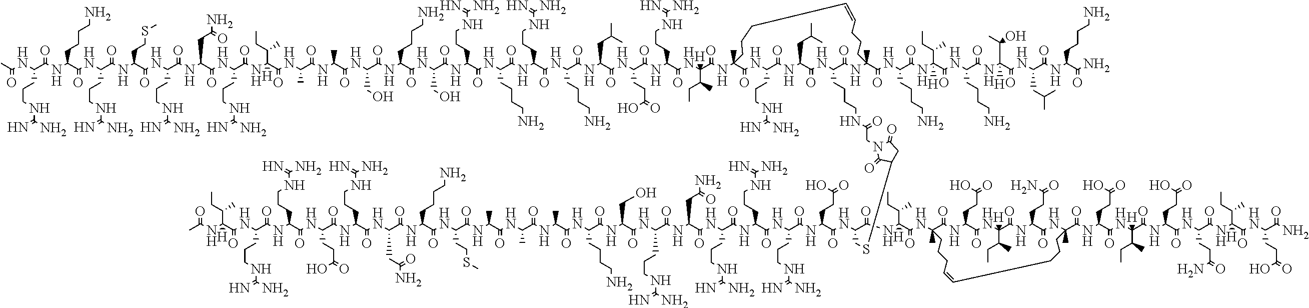

Further disclosed is an engineered DNA-binding dimer having a formula or structure depicted in any one of FIGS. 9-86.

Also disclosed, in some aspects, is a method for modifying expression of a gene in a cell, the method comprising providing to the cell an engineered DNA-binding dimer of the present disclosure. Further disclosed, in some aspects, is a method for treating a subject for a condition, the method comprising administering to the subject an effective amount of an engineered DNA-binding dimer of the present disclosure.

In some aspects, the condition is fibrosis. In some aspects, the fibrosis is liver fibrosis, renal fibrosis, cardiac fibrosis, pulmonary fibrosis, idiopathic pulmonary fibrosis (IPF), scleroderma, psoriasis, or myelofibrosis. In some aspects, the condition is diabetes. In some aspects, the condition is type 1 diabetes. In some aspects, the condition is type 2 diabetes. In some aspects, the condition is cancer. In some aspects, the cancer is leukemia, lymphoma, myeloma, triple negative breast cancer, prostate cancer, pancreatic neuroendocrine tumors, pancreatic ductal adenocarcinoma, ovarian cancer, lung adenocarcinoma, liver cancer, glioblastoma, or renal cell carcinoma. In some aspects, the cancer is breast cancer. In some aspects, the breast cancer is triple negative breast cancer. In some aspects, the method further comprises administering to the subject an additional cancer therapy. In some aspects, the additional cancer therapy is chemotherapy, radiotherapy, immunotherapy, or a proteasome inhibitor. In some aspects, the subject was previously treated with a cancer therapy. In some aspects, the subject was determined to be resistant to the cancer therapy. In some aspects, the cancer therapy is chemotherapy, radiotherapy, or immunotherapy.

Disclosed herein, in some aspects, is an engineered peptide having the sequence: Ac-IRRERNKMAAAKSRNRRRECTDTLEDETDQLE-NH2 (SEQ ID NO:59), Ac-IRRERNKMAAAKSRNRRRECLDELQAEIEQLE-NH2 (SEQ ID NO:60), Ac-IRRERNKMAAAKSRNRRRECIDELQAEIEQLE-NH2 (SEQ ID NO:61), Ac-IRRERNKMAAAKSRNRRRECIDEIQAEIEQIE-NH2 (SEQ ID NO:62), Ac-IRR#RNK#AAAKSRNRRRECLDELQAEIEQLE-NH2 (SEQ ID NO:63), Ac-IRRERNKMAAAKSRNRRRECL#ELQ#EIEQLE-NH2 (SEQ ID NO:64), Ac-IRR#RNK#AAAKSRNRRRECIDELQAEIEQLE-NH2 (SEQ ID NO:65), Ac-IRRERNKMAAAKSRNRRRECI#ELQ#EIEQLE-NH2 (SEQ ID NO:66), Ac-IRR#RNK#AAAKSRNRRRECIDEIQAEIEQIE-NH2 (SEQ ID NO:67), Ac-IRRERNKMAAAKSRNRRRECI#EIQ#EIEQIE-NH2 (SEQ ID NO:68), Ac-EEKRRIRRERNKMAAAKSRNRRRECLDELQAEIEQLE-NH2 (SEQ ID NO:69), Ac-EEKRRIRR#RNK#AAAKSRNRRRECLDELQAEIEQLE-NH2 (SEQ ID NO:70), or Ac-EEKRRIRRERNKMAAAKSRNRRRECL#ELQ#EIEQLE-NH2 (SEQ ID NO:71), wherein Ac is acetyl; and # is (S)-2-(4′-pentenyl)alanine. In some aspects, the engineered peptide has the sequence Ac-IRRERNKMAAAKSRNRRRECI#EIQ#EIEQIE-NH₂ (SEQ ID NO:68).

Disclosed herein, in some aspects, is an engineered peptide having the sequence: Ac-RKRMRNRIAASKSRKRKLERIAKmLEEKVKTLK-NH2 (SEQ ID NO:72), Ac-RKRMRNRIAASKSRKRKLERIARLKmEKVKTLK-NH2 (SEQ ID NO:73), Ac-RKRMRNRIAASKSRKRKLERAAELKmEKVATLK-NH2 (SEQ ID NO:74), Ac-RKRMRNRIAASKSRKRKLERIARLKmEKIKTLK-NH2 (SEQ ID NO:75), Ac-RKRMRNRIAASKSRKRKLERIARIKmEKIKTIK-NH2 (SEQ ID NO:76), Ac-RKR#RNR#AASKSRKRKLERIARLKmEKVKTLK-NH2 (SEQ ID NO:77), Ac-RKRMRNRIAASKSRKRKLERI#RLKm#KVKTLK-NH2 (SEQ ID NO:78), Ac-RKR#RNR#AASKSRKRKLERIARLKmEKIKTLK-NH2 (SEQ ID NO:79), Ac-RKRMRNRIAASKSRKRKLERI#RLKm#KIKTLK-NH2 (SEQ ID NO:80), Ac-RIKAERKRMRNRIAASKSRKRKLERIARLKmEKVKTLK-NH2 (SEQ ID NO:81), Ac-RIKAERKR#RNR#AASKSRKRKLERIARLKmEKVKTLK-NH2 (SEQ ID NO:82), or Ac-RIKAERKRMRNRIAASKSRKRKLERI#RLKm#KVKTLK-NH2 (SEQ ID NO:83), wherein Ac is acetyl; # is (S)-2-(4′-pentenyl)alanine; and Km is Lys(Mmt) or a Lys residue linked to a maleimide linker. In some aspects, the engineered peptide has the sequence Ac-RKRMRNRIAASKSRKRKLERI#RLKm#KIKTLK-NH₂ (SEQ ID NO:80).

Disclosed herein, in some aspects, is an engineered peptide having the sequence: Ac-RRKLKNRVAAQTARDRKKACMSELEQQVVDLE-NH2 (SEQ ID NO:84), Ac-RRKLKNRVAAQTARDRKKACLSELEQQVVDLE-NH2 (SEQ ID NO:85), Ac-RRKLKNRVAAQTARDRKKACL#ELE #QVVDLE-NH2 (SEQ ID NO:86), or Ac-RRK#KNR#AAQTARDRKKACLSELEQQVVDLE-NH2 (SEQ ID NO:87), wherein Ac is acetyl; and # is (S)-2-(4′-pentenyl)alanine. In some aspects, the engineered peptide has the sequence Ac-RRK#KNR#AAQTARDRKKACLSELEQQVVDLE-NH₂ (SEQ ID NO:87).

Disclosed herein, in some aspects, is an engineered peptide having the sequence: Ac-RRKLKNRVAAQTARDRKKARMSELKmQQVVDLE-NH2 (SEQ ID NO:88), Ac-RRKLKNRVAAQTARDRKKARLSELKmQQVVDLE-NH2 (SEQ ID NO:89), Ac-RRKLKNRVAAQTARDRKKARL#ELKm#QVVDLE-NH2 (SEQ ID NO:90), or Ac-RRK#KNR#AAQTARDRKKARLSELKmQQVVDLE-NH2 (SEQ ID NO:91), wherein Ac is acetyl; # is (S)-2-(4′-pentenyl)alanine; and Km is Lys(Mmt) or a Lys residue linked to a maleimide linker. In some aspects, the engineered peptide has the sequence Ac-RRK#KNR#AAQTARDRKKARLSELKₘQQVVDLE-NH₂ (SEQ ID NO:91).

Disclosed herein, in some aspects, is an engineered peptide having the sequence: Ac-KKMEQNKTAATRYRQKKRACQEALTGELKELE-NH2 (SEQ ID NO:92), Ac-KKMEQNKTAATRYRQKKRACLEALKAELKELR-NH2 (SEQ ID NO:93), Ac-KKMEQNKTAATRYRQKKRACL#ALK#ELKELR-NH2 (SEQ ID NO:94), wherein Ac is acetyl; and # is (S)-2-(4′-pentenyl)alanine. In some aspects, the engineered peptide has the sequence Ac-KKMEQNKTAATRYRQKKRACQEALTGELKELE-NH₂ (SEQ ID NO:92).

Disclosed herein, in some aspects, is an engineered peptide having the sequence: Ac-KKMEQNKTAATRYRQKKRAEQEALKmGELKELE-NH2 (SEQ ID NO:95), Ac-KKMEQNKTAATRYRQKKRAELEALKmAELKELR-NH2 (SEQ ID NO:96), or Ac-KKMEQNKTAATRYRQKKRAEL#ALKm#ELKELR-NH2 (SEQ ID NO:97), wherein Ac is acetyl; # is (S)-2-(4′-pentenyl)alanine; and Km is Lys(Mmt) or a Lys residue linked to a maleimide linker. In some aspects, the engineered peptide has the sequence Ac-KKMEQNKTAATRYRQKKRAEL#ALKₘ#ELKELR-NH₂ (SEQ ID NO:97).

Disclosed herein, in some aspects, is an engineered peptide having the sequence: Ac-IRRERNNIAVRKSRDKAKMCLLELQHKVLELR-NH2 (SEQ ID NO:98), or Ac-IRRERNNIAVRKSRDKAKMCL#ELQ#KVLELR-NH2 (SEQ ID NO:99), wherein Ac is acetyl; and # is (S)-2-(4′-pentenyl)alanine. In some aspects, the engineered peptide has the sequence Ac-IRRERNNIAVRKSRDKAKMCL#ELQ#KVLELR-NH₂ (SEQ ID NO:99).

Also disclosed is a composition comprising any two of the engineered peptides disclosed herein. Further disclosed is a method for generating an engineered DNA-binding dimer, the method comprising subjecting such a composition to conditions sufficient to form a side-by-side interpeptide linkage between the two engineered peptides. The conditions may comprise, for example, providing 2-(2,5-dioxo-2,5-dihydro-1H-pyrrol-1-yl)acetic acid.

Further disclosed herein, in some aspects, is a method of reducing expression of a HIF1α target gene in a cell, the method comprising providing to the cell an engineered DNA-binding dimer comprising: (a) a first engineered peptide comprising (i) a modified basic domain sequence of XBP1 and (ii) a modified leucine zipper domain sequence of XBP1; and, (b) a second engineered peptide linked to the first engineered peptide via a side-by-side interpeptide linkage, the second engineered peptide comprising (i) a modified basic domain sequence of XBP1 and (ii) a modified leucine zipper domain sequence of XBP1. In some aspects, the engineered DNA-binding dimer is provided in an amount effective to reduce expression of GLUT1 in the cell. In some aspects, the engineered DNA-binding dimer is provided in an amount effective to reduce expression of VEGFA in the cell. In some aspects, the engineered DNA-binding dimer is provided in an amount effective to reduce expression of PGK1 in the cell. In some aspects, the cell is a cancer cell. In some aspects, the cell is a breast cancer cell. In some aspects, the cell is a triple negative breast cancer cell.

In some aspects, the engineered DNA-binding dimer has formula:

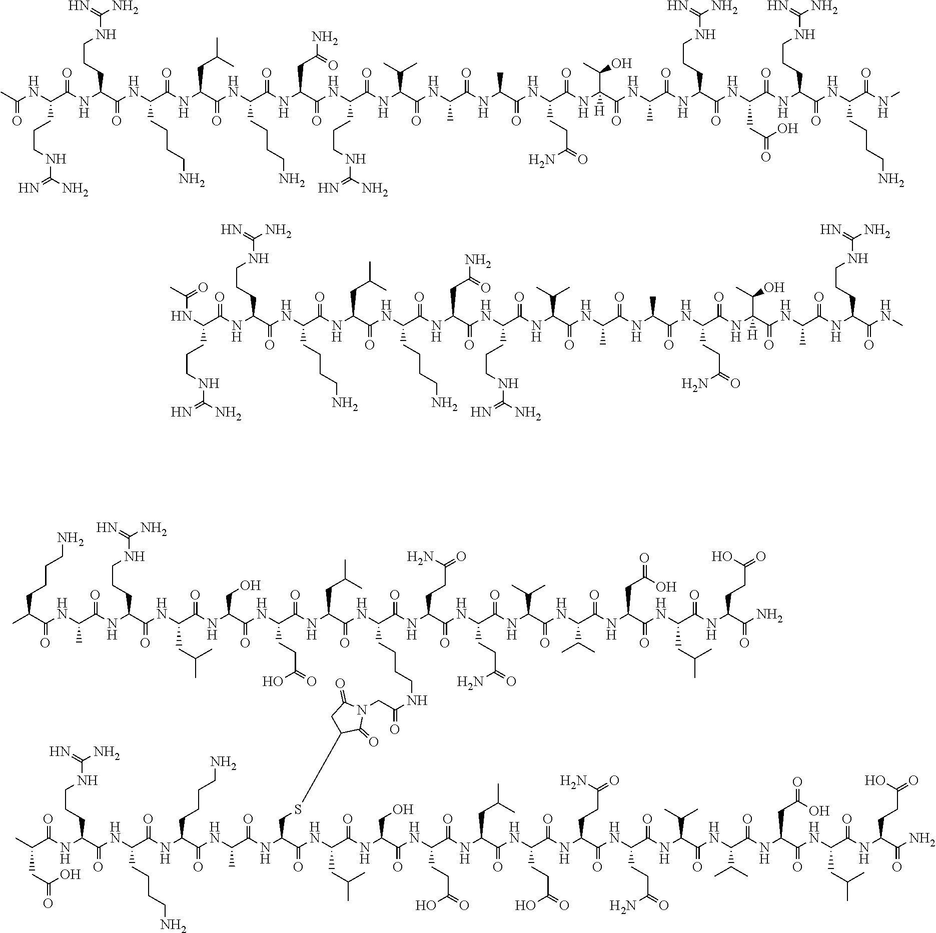

In some aspects, the engineered DNA-binding dimer has formula.

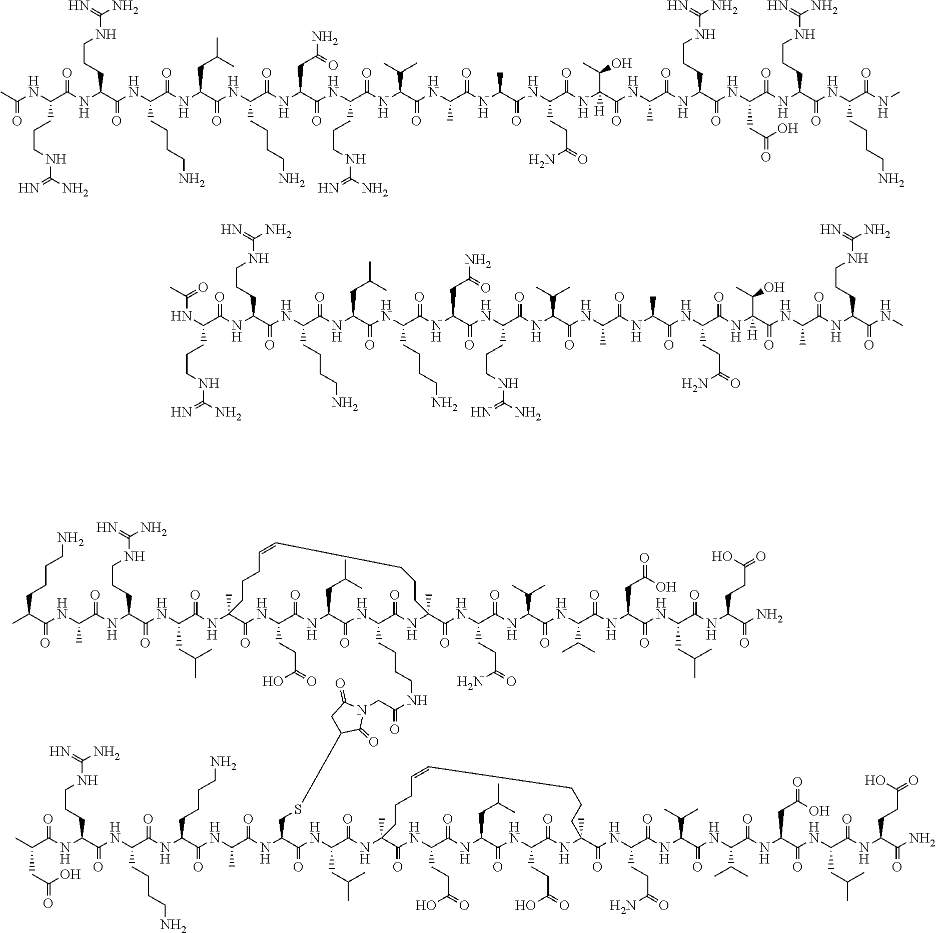

In some aspects, the engineered DNA-binding dimer has formula

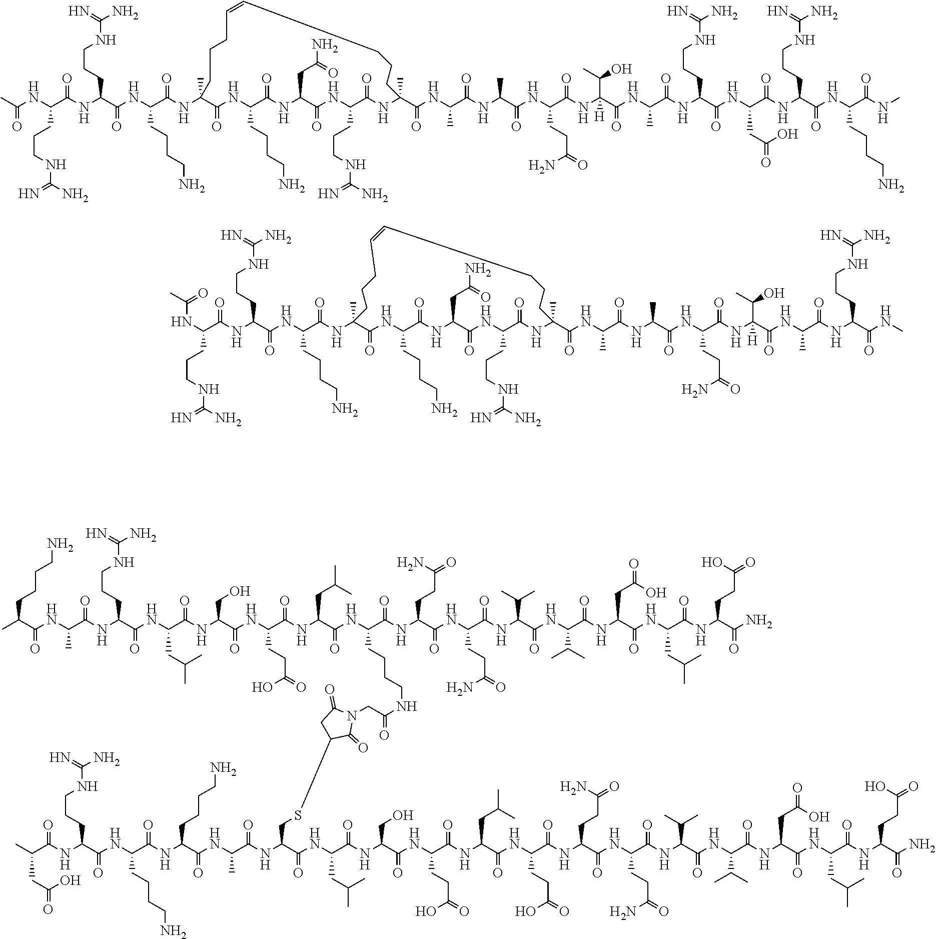

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention.

“Individual, “subject,” and “patient” are used interchangeably and can refer to a human or non-human.

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

It is specifically contemplated that any limitation discussed with respect to one aspect of the invention may apply to any other aspect of the invention. Furthermore, any composition of the invention may be used in any method of the invention, and any method of the invention may be used to produce or to utilize any composition of the invention. Any aspect discussed with respect to one aspect of the disclosure applies to other aspects of the disclosure as well and vice versa. For example, any step in a method described herein can apply to any other method. Moreover, any method described herein may have an exclusion of any step or combination of steps. Aspects of an aspect set forth in the Examples are also aspects that may be implemented in the context of aspects discussed elsewhere in a different Example or elsewhere in the application, such as in the Summary, Detailed Description, Claims, and Brief Description of the Drawings.

It is contemplated that any aspect discussed in this specification can be implemented with respect to any method or composition of the invention, and vice versa. Furthermore, compositions of the invention can be used to achieve methods of the invention.

Other objects, features and advantages of the present invention will become apparent from the following detailed description. It should be understood, however, that the detailed description and the specific examples, while indicating specific aspects of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific aspects presented herein.

FIG. 1 shows results from an electrophoretic mobility shift assay (EMSA) demonstrating binding of bZIP transcriptional repressor STR4 to the UPR element (UPRE).

FIG. 2 shows results from an EMSA demonstrating binding of bZIP transcriptional repressor STR22 to the UPRE with K_{D}=14.17 nM.

FIG. 3 shows results from an EMSA demonstrating binding of bZIP transcriptional repressor STR22 to the shown consensus sequence (Kᵢ 28.59) but not to the shown mutant sequence.

FIG. 4 shows fluorescent microscopy results demonstrating penetration of HeLa cells by bZIP transcriptional repressor STR4 at 5 μM.

FIG. 5 shows fluorescent microscopy results demonstrating penetration of HeLa cells by bZIP transcriptional repressor STR22 at 5 μM, which is sustained over 24 hours after administration.

FIG. 6 shows expression of an XBP1 transcriptionally driven luciferase reporter in HeLa cells administered FLAG-XBP1s and varying concentrations of bZIP transcriptional repressor STR22 (2.5-20 μM) as shown for 12 hours. The results demonstrate inhibition of luciferase expression with STR22.

FIG. 7 shows expression of an XBP1 transcriptionally driven luciferase reporter in HeLa cells administered tunicamycin and varying concentrations of bZIP transcriptional repressor STR22 (2.5-20 μM) as shown.

FIG. 8 shows qPCR expression data for the shown genes (SEC23B, SERP1, EDEM1, DNAJB9) from HeLa cells treated with tunicamycin (5000 ng/ml for 12 hours) and varying concentrations of bZIP transcriptional repressor STR22 (2.5-20 PM) as shown. STR22 treatment occurred for 36 hours, starting 24 hours before tunicamycin treatment.

FIG. 9 shows qPCR expression data for the shown genes (SEC23B, SERP1, EDEM1, DNAJB9) from HeLa cells treated with tunicamycin (5000 ng/ml for 12 hours) and 20 μM of bZIP transcriptional repressor STR22 for varying amounts of time (12-36 hours) as shown.

FIG. 10 shows qPCR expression data for the shown genes (DNAJB9, EDEM1, SERP1, SEC23B) treated with tunicamycin (5000 ng/ml for 12 hours) and 20 μM of bZIP transcriptional repressor STR22 for varying amounts of time (12-36 hours) as shown.

FIG. 11 shows qPCR expression data for the shown genes (OCT4, PGK1, VEGFA, GLUT1) from HeLa cells treated under normoxia (5% 02) or hypoxia (1% 02) for 24 hours, together with varying concentrations of bZIP transcriptional repressor STR22 as shown (2.5-20 μM). Cells were treated with STR22 for 48 hours, starting at 24 hours before hypoxia treatment.

FIG. 12 shows results from an EMSA demonstrating binding of bZIP transcriptional repressor FJSTR72 to the AP-1 site with K_{D}=12 nM.

FIG. 13 shows fluorescent microscopy results demonstrating penetration of cells by bZIP transcriptional repressor FJSTR7 at 5 μM.

FIG. 14 shows fluorescent microscopy results demonstrating penetration of cells by bZIP transcriptional repressor FJSTR71 at 2 μM.

FIG. 15 shows fluorescent microscopy results demonstrating penetration of cells by bZIP transcriptional repressor FJSTR72 at 5 μM.

FIGS. 16A-16B show in vitro and in vivo effects of FJSTR72. FIG. 16A shows MCF-7 cells treated with FJSTR72 for 8 hours at 10, 5, 2.5, 1.25 μM resulting in an IC₅₀ of 2.0 μM. FIG. 16B shows tumor volume from subcutaneous MC38-bearing C57BL/8 mouse models. When the tumors reached ˜100 mm3, either 10 mL of FJSTR72 (2 mg/ml) in water or 10 mL of PBS was intratumorally injected into the mice.

FIG. 17 shows results from an EMSA analyzing DNA binding of bZIP transcriptional repressor CASTR4.

FIG. 18 shows shows results from an EMSA analyzing DNA binding of bZIP transcriptional repressor CASTR41.

FIG. 19 shows shows results from an EMSA analyzing DNA binding of bZIP transcriptional repressor ASTR4.

FIG. 20 shows shows results from an EMSA analyzing DNA binding of bZIP transcriptional repressor ASTR41.

FIG. 21 shows example synthetic dimers of the present disclosure and DNA binding results measured by electrophoretic mobility shift assay.

FIG. 22 shows the chemical structure of bZIP transcriptional repressor STR1.

FIG. 23 shows the chemical structure of bZIP transcriptional repressor STR2.

FIG. 24 shows the chemical structure of bZIP transcriptional repressor STR3.

FIG. 25 shows the chemical structure of bZIP transcriptional repressor STR4.

FIG. 26 shows the chemical structure of bZIP transcriptional repressor STR21.

FIG. 27 shows the chemical structure of bZIP transcriptional repressor STR22.

FIG. 28 shows example synthetic dimers of the present disclosure and DNA binding results measured by electrophoretic mobility shift assay.

FIG. 29 shows the chemical structure of bZIP transcriptional repressor ASTR1.

FIG. 30 shows the chemical structure of bZIP transcriptional repressor ASTR2.

FIG. 31 shows the chemical structure of bZIP transcriptional repressor ASTR3.

FIG. 32 shows the chemical structure of bZIP transcriptional repressor ASTR4.

FIG. 33 shows the chemical structure of bZIP transcriptional repressor ASTR41.

FIG. 34 shows example synthetic dimers of the present disclosure and DNA binding results measured by electrophoretic mobility shift assay.

FIG. 35 shows the chemical structure of bZIP transcriptional repressor CASTR4.

FIG. 36 shows the chemical structure of bZIP transcriptional repressor CASTR41.

FIG. 37 shows example synthetic dimers of the present disclosure and DNA binding results measured by electrophoretic mobility shift assay.

FIG. 38 shows the chemical structure of bZIP transcriptional repressor FJSTR1.

FIG. 39 shows the chemical structure of bZIP transcriptional repressor FJSTR2.

FIG. 40 shows the chemical structure of bZIP transcriptional repressor FJSTR3.

FIG. 41 shows the chemical structure of bZIP transcriptional repressor FJSTR4.

FIG. 42 shows example synthetic dimers of the present disclosure and DNA binding results measured by electrophoretic mobility shift assay.

FIG. 43 shows the chemical structure of bZIP transcriptional repressor FJSTR5.

FIG. 44 shows the chemical structure of bZIP transcriptional repressor FJSTR6.

FIG. 45 shows the chemical structure of bZIP transcriptional repressor FJSTR7.

FIG. 46 shows the chemical structure of bZIP transcriptional repressor FJSTR8.

FIG. 47 shows the chemical structure of bZIP transcriptional repressor FJSTR9.

FIG. 48 shows the chemical structure of bZIP transcriptional repressor FJSTR10.

FIG. 49 shows example synthetic dimers of the present disclosure and DNA binding results measured by electrophoretic mobility shift assay.

FIG. 50 shows the chemical structure of bZIP transcriptional repressor FJSTR31.

FIG. 51 shows the chemical structure of bZIP transcriptional repressor FJSTR32.

FIG. 52 shows example synthetic dimers of the present disclosure and DNA binding results measured by electrophoretic mobility shift assay.

FIG. 53 shows the chemical structure of bZIP transcriptional repressor FJSTR71.

FIG. 54 shows the chemical structure of bZIP transcriptional repressor FJSTR72.

FIG. 55 shows the chemical structure of bZIP transcriptional repressor FJSTR91.

FIG. 56 shows the chemical structure of bZIP transcriptional repressor FJSTR92.

FIG. 57 shows example synthetic dimers of the present disclosure and DNA binding results measured by electrophoretic mobility shift assay. In some aspects, the synthetic dimers of FIG. 57 are poor binders to DNA and/or do not bind DNA stably, specifically, or at all.

FIG. 58 shows the chemical structure of synthetic dimer FJ131.

FIG. 59 shows the chemical structure of synthetic dimer FJ132.

FIG. 60 shows the chemical structure of synthetic dimer FJ133.

FIG. 61 shows the chemical structure of synthetic dimer FJ134.

FIG. 62 shows the chemical structure of synthetic dimer FJ135.

FIG. 63 shows example synthetic dimers of the present disclosure and DNA binding results measured by electrophoretic mobility shift assay. In some aspects, the synthetic dimers of FIG. 63 are poor binders to DNA and/or do not bind DNA stably, specifically, or at all.

FIG. 64 shows the chemical structure of synthetic dimer FJ111.

FIG. 65 shows the chemical structure of synthetic dimer FJ112.

FIG. 66 shows the chemical structure of synthetic dimer FJ113.

FIG. 67 shows the chemical structure of synthetic dimer FJ114.

FIG. 68 shows the chemical structure of synthetic dimer FJ115.

FIG. 69 shows example synthetic dimers of the present disclosure and DNA binding results measured by electrophoretic mobility shift assay. In some aspects, the synthetic dimers of FIG. 69 are poor binders to DNA and/or do not bind DNA stably, specifically, or at all.

FIG. 70 shows the chemical structure of synthetic dimer FJ121.

FIG. 71 shows the chemical structure of synthetic dimer FJ122.

FIG. 72 shows the chemical structure of synthetic dimer FJ123.

FIG. 73 shows the chemical structure of synthetic dimer FJ124.

FIG. 74 shows the chemical structure of synthetic dimer FJ125.

FIG. 75 shows example synthetic dimers of the present disclosure and DNA binding results measured by electrophoretic mobility shift assay. In some aspects, the synthetic dimers of FIG. 75 are poor binders to DNA and/or do not bind DNA stably, specifically, or at all.

FIG. 76 shows the chemical structure of synthetic dimer FJ181.

FIG. 77 shows the chemical structure of synthetic dimer FJ182.

FIG. 78 shows the chemical structure of synthetic dimer FJ183.

FIG. 79 shows the chemical structure of synthetic dimer FJ184.

FIG. 80 shows the chemical structure of synthetic dimer FJ185.

FIG. 81 shows example synthetic dimers of the present disclosure and DNA binding results measured by electrophoretic mobility shift assay. In some aspects, the synthetic dimers of FIG. 81 are poor binders to DNA and/or do not bind DNA stably, specifically, or at all.

FIG. 82 shows the chemical structure of synthetic dimer FJ191.

FIG. 83 shows the chemical structure of synthetic dimer FJ192.

FIG. 84 shows the chemical structure of synthetic dimer FJ193.

FIG. 85 shows the chemical structure of synthetic dimer FJ194.

FIG. 86 shows the chemical structure of synthetic dimer FJ195.

FIG. 87 shows example synthetic dimers of the present disclosure and DNA binding results measured by electrophoretic mobility shift assay. In some aspects, the synthetic dimers of FIG. 87 are poor binders to DNA and/or do not bind DNA stably, specifically, or at all.

FIG. 88 shows the chemical structure of synthetic dimer FJ161.

FIG. 89 shows the chemical structure of synthetic dimer FJ162.

FIG. 90 shows the chemical structure of synthetic dimer FJ163.

FIG. 91 shows the chemical structure of synthetic dimer FJ164.

FIG. 92 shows the chemical structure of synthetic dimer FJ165.

FIG. 93 shows example synthetic dimers of the present disclosure and DNA binding results measured by electrophoretic mobility shift assay. In some aspects, the synthetic dimers of FIG. 93 are poor binders to DNA and/or do not bind DNA stably, specifically, or at all.

FIG. 94 shows the chemical structure of synthetic dimer FJ171.

FIG. 95 shows the chemical structure of synthetic dimer FJ172.

FIG. 96 shows the chemical structure of synthetic dimer FJ173.

FIG. 97 shows the chemical structure of synthetic dimer FJ174.

FIG. 98 shows the chemical structure of synthetic dimer FJ175.

FIGS. 99A-99C show inhibition of HIF1α DNA binding and target gene expression by bZIP transcriptional repressor STR22. FIG. 99A shows western blot analysis of HIF1α induction after 6 hr of hypoxia (1% 02) in the presence or absence of STR22 treatment. FIG. 99B shows qPCR quantification of hypoxia-induced target genes in HeLa cells with and without indicated doses of STR22. FIG. 99C shows ChIP-qPCR of HIF1α binding to HRE-containing genes in the presence and absence of hypoxia and STR22 treatment. For all of FIGS. 56A-56C, *, **, *** and **** refer to student's t-test p-value <0.05, <0.01, <0.001 and <0.0001, respectively.

FIGS. 100A-100C show inhibition of hypoxic gene expression and invasion in triple-negative breast cancer (TNBC) cells by bZIP transcriptional repressor STR22. FIG. 100A shows qPCR expression of hypoxia-induced and HRE-regulated target genes in MDA-MB231 TNBC cells with or without STR22 treatment (20 μM). FIG. 100B shows MDA-MB231 cell viability with or without STR22 treatment (20 μM). FIG. 100C shows invasion of MDA-MB231 cells with or without STR22 treatment (20 μM for 24 hours). *** and **** represent student's t-test p<0.001 and 0.0001, respectively.

FIGS. 101A-101G show design and synthesis of a potent and specific synthetic transcriptional repressors derived from XBP1. FIG. 101A, The active, spliced form of XBP1 (XBP1s) forms a canonical bZIP homodimer to bind UPRE (blue) and embedded HRE (red) DNA sequences. The bZIP structure shown is from a crystal structure of the homologous bZIP JUN homodimer bound to target DNA (PDB: 2H7H). FIG. 101B, Schematic overview of individual stabilization design elements in the creation of XBP1-derived STRs to target UPRE/HRE-DNA sites. The inventors hypothesized that when suitably combined these elements could generate hyperstable, minimal transcription factor mimetics. FIG. 101C, Chemical structures of STRs designed from the bZIP domain of XBP1. Interhelix ligation sites, optimized interfacial mutations and hydrocarbon macrocycle locations are depicted in blue, red and black, respectively, with representative linker structures shown below. FIGS. 101D-101E, Representative EMSA gel showing binding of STR22 to target (UPRE) and control non-target (AP-1) DNA across a range of doses (d) and binding curves (e) from EMSA experiments with individual STRs binding fluorophore-labeled UPRE-containing DNA from n=3 biological replicates. Data are shown as the mean±s.d. FIG. 101F, Representative confocal fluorescence microscopy images of HeLa cells treated with FITC-STR22 or FITC-STR4 for 12 hours. FIG. 101G, Cellular penetration and in situ stability of FITC-STR4 and FITC-STR22 were examined by fluorescence gel analysis.

FIGS. 102A-102E show STR22 inhibits XBP1s-DNA binding and target gene expression. FIG. 102A, Western blot analysis of FLAG-XBP1s following transient transfection and treatment with DMSO vehicle or STR22 (20 μM). FIG. 102B, 3×UPRE-regulated luciferase reporter activity in HeLa cells expressing Flag-XBP1s following treatment with DMSO or STR22 (two-fold dilutions between 20 to 2.5 μM, 24 hours). FIG. 102C, UPRE-regulated reporter activity as in FIG. 102B following induction of endogenous XBP1s with tunicamycin treatment (5 μg/mL; 12 hours) and treatment with DMSO or STR22 (24 hours). FIG. 102D, ChIP-qPCR quantification of Flag-XBP1s occupancy at UPRE-containing target genes in Flag-XBP1s-transfected HeLa cells treated with vehicle or STR22. FIG. 102E, mRNA levels of canonical XBP1-dependent target genes in Flag-XBP1s- or control plasmid-transfected HeLa cells treated with DMSO or STR22 treatment for 24 hours. Data shown in FIG. 102D are mean±s.d. from n=3 technical replicates. Data shown in FIG. 102B, FIG. 102C and FIG. 102E are the mean±s.d. from n=3 biological replicates. Statistical comparisons are Student's two-tailed t-tests: *, p<0.05; **, p<0.01; ***, p<0.001; ****, p <0.0001.

FIGS. 103A-103H show STR22 globally suppresses HIF1α-DNA binding and hypoxia-induced gene expression. FIG. 103A, Western blot analysis of HIF1α protein levels following DMSO or STR22 treatment (24 hours, 20 μM) in HeLa cells under normal or hypoxic (1% O2, 6 hours) culture conditions. FIG. 103B, 5×HRE-regulated firefly luciferase activity in HeLa cells treated with a range of STR22 doses for 24 hours under hypoxic conditions (1% O₂). FIG. 103C, RT-qPCR analysis of mRNA levels of known HRE-regulated genes with STR22 (48 hours, 20 μM) treatments under normoxic or hypoxic (1% 02, 24 hours) conditions in different knockout HeLa cell lines. Normalized expression is shown relative to normoxic condition. FIG. 103D, Genome-wide changes in HIF1α-bound loci measured by ChIP-seq peaks from HeLa cells under normoxia or hypoxia (1% 02 for 6 hours) following 24 hours DMSO or STR22 treatment. Upper right logo plot depicts the most enriched HIF1α motif in hypoxic cells treated with DMSO; 99% of these sites are lost or decreased with STR22 treatment. The lower right logo plot depicts a less defined HIF1α-bound motif induced by STR22 treatment. FIG. 103E, HIF1α ChIP-seq read-density heat maps from HeLa cells depicted in FIG. 103A. FIG. 103F, Track view of HIF1α-density profiles of representative HRE-regulated gene loci from HeLa cells under indicated oxygen and compound treatment. FIG. 103G, GSEA plots generated from mRNA-seq profiles comparing global gene expression in HeLa cells treated with DMSO under normoxic or hypoxic conditions, as well as STR22 under hypoxic conditions. Hallmark hypoxia response genes are significantly enriched in hypoxic versus normoxic cells (top), and significantly downregulated in hypoxic cells treated with STR22 relative to vehicle (bottom). FIG. 103H, Heat map representation of top hypoxia response mRNA transcript levels in HeLa cells under normoxia, hypoxia and hypoxia with STR22 treatment. Data shown in FIG. 103B and FIG. 103C are the mean±s.d. from n=3 biological replicates. Statistical comparisons are Student's two-tailed t-tests: *, p<0.05; **, p <0.01; ***, p<0.001; ****, p<0.0001. ChIP-Seq data in FIGS. 103A-103C are representative of n=2 biological replicates. mRNA-Seq data in FIGS. 103D-103E are derived from n=3 biological replicates.

FIGS. 104A-104E show STR22 treatment inhibits aggressive TNBC cell phenotypes in vitro and in vivo. FIG. 104A, Relative Boyden chamber invasion of MDA-MB-231 and SUM159 cells under hypoxia for 24 hours with or without STR22 treatment. FIG. 104B, Relative growth of MDA-MB-231 and SUM159 cells treated with vehicle or STR22 under normoxia or hypoxia. FIG. 104C, Experiment design (left) and relative growth curves (right) of MDA-MB-231 tumors engrafted onto mammary fat pads of nude mice treated twice per week with intratumoral vehicle or STR22 (20 μg). FIG. 104D, RT-qPCR analysis of target gene expression from tumors in FIG. 104C 24 hours after final dose. Relative mRNA expression is normalized to control RPL13A. FIG. 104E, Schematic depicting the dual regulation of stress-responsive gene expression by XBP1s and HIF1α at UPRE and HRE sites (left). XBP1-derived STRs directly recognize and bind UPRE and HRE sites in tumor cells, thereby preventing endogenous XBP1 and HIF1α and activation of oncogenic gene expression and phenotypes (right). Data in FIG. 104A-104B represent mean±s.d. from n=3 biological replicates; data in FIG. 104C are mean±s.e.m. from n=10 mice per group; FIG. 104D are mean±s.e.m. from n=10 (veh.) and n=9 (treatment group, one measurement excluded because target genes were not detected by qPCR) mice per group. Statistical comparisons are Student's two-tailed t-tests: *, p<0.05; **, p<0.01; ***, p<0.001; ****, p<0.0001.

FIGS. 105A-105C show schematic synthesis of synthetic transcription repressors (STR). FIG. 105A, Convergent synthesis of STRs containing local and global nucleation and interhelix linker installation. Two branches of STR are synthesized on-resin with bisalkylated, terminal olefin containing ‘S5’ amino acids at defined positions for on-resin ring closing metathesis (1: Grubbs I catalyst, DCE, N2 atmosphere). One of helical branches harbors an orthogonal Lys(Mmt) at a defined C-terminal 8th position for deprotection and acylation with a specific maleimide linker 2:1% TFA/DCM; 3:0.1 M Mal-Gly-OH, 0.1 M HCTU, 0.2 M DIPEA, DMF, N2 atmosphere) Helices were then cleaved from the resin and purified through HPCL and dried using lyophilization. Stapled helices are ligated in aqueous solution (50% ACN/H2O, pH 7.0-7.2) and readily purified to yield fully synthetic transcription repressors (STRs). FIG. 105B, Representative chromatogram (left) and mass spectra (right) of STR22. FIG. 105C, Structure of STR22.

FIGS. 106A-106G show representative EMSA gels. FIG. 106A, Sequence of IRdye700-labeled oligo probes or free oligos. Blue: UPRE consensus. Red: mutated nucleotides from UPRE sequence. FIG. 106B, Representative EMSA gel of 8-500 nM STR1, STR2, STR3 and STR4 and 5 nM IRdye700-labelled UPRE oligo probe. FIGS. 106C-106F, Representative EMSA gels and quantification curves for STR21 (c, e) and STR22 (d, f) binding to UPRE or AP-1 oligos (c, d) or 5 nM labeled UPRE oligo with 7-100 nM free-labeled UPRE consensus or mutant oligos (e, f). Red: IRdye700-labeled UPRE oligo probe or UPRE consensus, blue: AP-1 probe or UPRE mutant. Data is shown as mean±s.d. from biological triplicates. g, qEMSA profile of STR22 binding to ˜40 distinct operator motif sequences, with the embedded HRE/UPRE sequence 5′-TGACGTGG-3′ being the most enriched in representative replicate experiments.

FIGS. 107A-107B show optimized STRs have a higher cellular uptake and stability and inhibit tunicamycin-induced UPRE-regulated gene expression. FIG. 107A, Representative fluorescent confocal microscopy images of HeLa cells treated with FITC-STR4 and FITC-STR22 for the indicated timepoints. Scale bar=20 μm. FIG. 107B, RT-qPCR quantification of mRNA levels of known XBP1s-dependent target genes in tunicamycin-treated (12 hours) HeLa cells with or without STR22 treatment (20 μM, 36 hours).

FIGS. 108A-108B show XBP1s or HIF1α was knocked out in respective HeLa knockout cell lines and HIF1α was upregulated under hypoxia. FIG. 108A, Western blot analysis of HIF1α (left) or XBP1s under hypoxia (1% 02, 6 hours) or tunicamycin (5000 ng/ml, 6 hours). FIG. 108B, Western blot analysis of HIF1α under hypoxia (1% 02, 6 hours) in MDA-MB-231 (left) and SUM159 (right) cell lines.

FIGS. 109A-109D show STR22 directly competes with HIF1α and inhibits HIF1α-regulated gene expression. FIG. 109A, ChIP-qPCR quantification of HIF1α binding to HRE-containing genes in the presence and absence of hypoxia (1% 02, 6 hours) and STR22 treatment (24 hours, 20 μM). α-NRS, normal rabbit serum. FIG. 109B, RT-qPCR analysis of HRE-regulated mRNA levels with DMSO or STR22 (48 hours, 20 PM) treatments under normoxic or hypoxic (1% 02, 24 hours) conditions. FIG. 109C, FIG. 109D, RT-qPCR quantification of mRNA levels of HRE-regulated target genes with DMSO or STR22 treatment (20 μM, 24 hours) under normoxic or hypoxic conditions (1% 02, 24 hours) in MDA-MB-231 cells (FIG. 109C) and SUM159 cells (FIG. 109D). Data in FIG. 109B, FIG. 109C, and FIG. 109D are normalized qPCR data are relative to RPL13A. Data in FIG. 109A, FIG. 109C and FIG. 109D are shown as the mean±s.d. of n=3 technical replicates. Data in FIG. 109B are shown as the mean±s.d. of n=3 biological replicates. Statistical comparisons are Student's two-tailed t-tests: *, p<0.05; **, p<0.01; ***, p<0.001; ****, p<0.0001.

FIGS. 110A-110G show STR22 globally suppresses HIF1α-DNA binding and hypoxia-dependent gene expression. FIG. 110A, Snapshot of the second replicate of HIF1α ChIP-seq with/without STR22 treatment. FIG. 110B, RNA-seq samples are clustered based on distance between each sample. FIGS. 110C-110D, Heat map representation of classical hypoxia response mRNA transcript levels (FIG. 110B) and canonical hypoxia response pathway (FIG. 110C) in HeLa cells under normoxia, hypoxia and hypoxia with STR22 treatment. FIGS. 110E-110F, GSEA plots generated from mRNA-seq profiles comparing global gene expression in HeLa cells treated with DMSO under normoxic or hypoxic conditions, as well as STR22 under hypoxic conditions. BIOCATRA_HIF_pathway (FIG. 110D) and Response_to_Hypoxia (FIG. 110E) genes are significantly enriched in hypoxic versus normoxic cells (top), and significantly downregulated in hypoxic cells treated with STR22 relative to vehicle (bottom). p<0.0001 in both GSEA analyses. FIG. 110G, Ingenuity pathway analysis of hypoxia vs normoxia (up) and hypoxia+STR22 vs hypoxia (down) mRNA-seq samples. Gene targets in HIF1α-pathway and related biological functions are listed. Scale bar is shown as the fold increase (red) or decrease (green) of transcripts level.

FIGS. 111A-111F show STR22 inhibits TNBC cells and malignant phenotypes in vivo. FIG. 111A, Dissected tumors after vehicle (top) or STR22 (bottom) treatment (n=10 per group). FIGS. 111B-111C, Volume of tumors before (FIG. 111C) or after (FIG. 111C) vehicle or STR22 treatment. FIG. 111D, Mass of tumors after vehicle or STR22 treatment. FIG. 111E, Growth of MDA-MB-231 tumors engrafted onto mammary fat pad of nude mice treated twice per week with intratumoral vehicle or STR22 (20 μg, n=4 per group). FIG. 111F, RT-PCR analysis of target gene expression from tumors in (e) 24 hours after the final dose. Relative mRNA expression is first normalized to RPL13A and then normalized to average of vehicle treatment (n=4 per group).

FIG. 112 shows MS signal detection of STR22 at the provided concentrations, added to 10× diluted plasma.

FIG. 113 shows the detection of STR22 after IP and IV administration of representative STR22 to healthy mice.

FIG. 114 shows the measured body weight of healthy mice administered STR22 at 25 mg/kg, 10 mg/kg, and 5 mg/kg, subcutaneously or intraveneously.

FIG. 115 shows example synthetic dimers of the present disclosure and DNA binding results measured by electrophoretic mobility shift assay.

FIG. 116 shows the chemical structure of bZIP transcriptional repressor STR5.

FIG. 117 shows the chemical structure of bZIP transcriptional repressor STR6.

FIG. 118 shows example synthetic dimers of the present disclosure and DNA binding results measured by electrophoretic mobility shift assay.

FIG. 119 shows the chemical structure of bZIP transcriptional repressor FJSTR11.

FIG. 120 shows the chemical structure of bZIP transcriptional repressor FJSTR12.

FIG. 121 shows the chemical structure of bZIP transcriptional repressor FJSTR121.

FIG. 122 shows the chemical structure of bZIP transcriptional repressor FJSTR122.

FIG. 123 shows example synthetic dimers of the present disclosure and DNA binding results measured by electrophoretic mobility shift assay.

FIG. 124 shows the chemical structure of synthetic dimer FJSTR181.

FIG. 125 shows the chemical structure of synthetic dimer FJSTR191.

FIG. 126 shows the chemical structure of synthetic dimer FJSTR201.

FIG. 127 shows example synthetic dimers of the present disclosure and DNA binding results measured by electrophoretic mobility shift assay.

FIG. 128 shows the chemical structure of synthetic dimer BMSTR2.

FIG. 129 shows the chemical structure of synthetic dimer BMSTR21.

FIG. 130 shows the chemical structure of synthetic dimer BMSTR22.

FIG. 131 shows example synthetic dimers of the present disclosure and DNA binding results measured by electrophoretic mobility shift assay.

FIG. 132 shows the chemical structure of synthetic dimer NMSTR2.

FIG. 133 shows the chemical structure of synthetic dimer NMSTR21.

FIG. 134 shows the chemical structure of synthetic dimer NMSTR22.

FIG. 135 shows example synthetic dimers of the present disclosure and DNA binding results measured by electrophoretic mobility shift assay.

FIG. 136 shows the chemical structure of synthetic dimer FJSTR181.

FIG. 137 shows the chemical structure of synthetic dimer FJSTR191.

FIG. 138 shows the chemical structure of synthetic dimer FJSTR201.

### DETAILED DESCRIPTION OF THE INVENTION

As described herein, the inventors have developed a modular strategy for generation of synthetic DNA binding molecules capable of competing for DNA binding with bZIP-containing transcription factors (zTFs). These molecules, described herein as “bZIP transcriptional repressors,” “engineered DNA-binding dimers,” or “synthetic transcriptional repressors,” may be generated using the methods and systems described herein using any zTF as a starting point, and thus may be used to compete for binding of any natural zTF. Various example bZIP transcriptional repressors are described herein, along certain examples methods for use, including in DNA binding and modification of gene expression. In some aspects, disclosed are engineered DNA-binding dimers capable of competing for binding with zTFs such as Fos/Jun heterodimers, XBP1 homodimers, ATF4 homodimers, and CEPBβ/ATF4 heterodimers.

The vast majority of oncogenic transcription factors (TFs) are perceived to be undruggable because of the difficulty in targeting extended protein-protein and protein-DNA interaction surfaces. Two of these proteins, XBP1 and HIF1α, are stress-responsive TFs that respond to and protect against cellular damage caused by dysregulated metabolism and microenvironmental conditions. Certain aspects herein relate to chemical strategies to create fully synthetic transcriptional repressors (STRs) that mimic one or more bZIP DNA-binding domains, such as those of XBP1. In some aspects, STR22, a synthesized bZIP-binding protein, binds XBP1- and HIF1α-target DNA sequences with high potency and specificity and, in some aspects, directly competes with both TFs at endogenous target gene promoters in cells. In certain aspects, under hypoxic conditions, STR22 globally suppresses HIF1α binding to hypoxia response element (HRE) promoters and enhancers and thereby inhibits hypoxia-induced gene expression. In certain aspects, such as in aspects involving triple negative breast cancer cells, STR22 blocks pro-tumorigenic phenotypes and hypoxia-induced stress protection in cell culture. In some in vivo aspects, where tumor hypoxia is more prevalent, STR22 treatment inhibited HIF1α-dependent gene expression and tumor growth. These data from aspects disclosed herein validate a novel strategy for dual targeting of two currently intractable TFs in TNBC and other cancers. Certain aspects also relate to a general strategy to develop antagonists for other bZIP TFs.

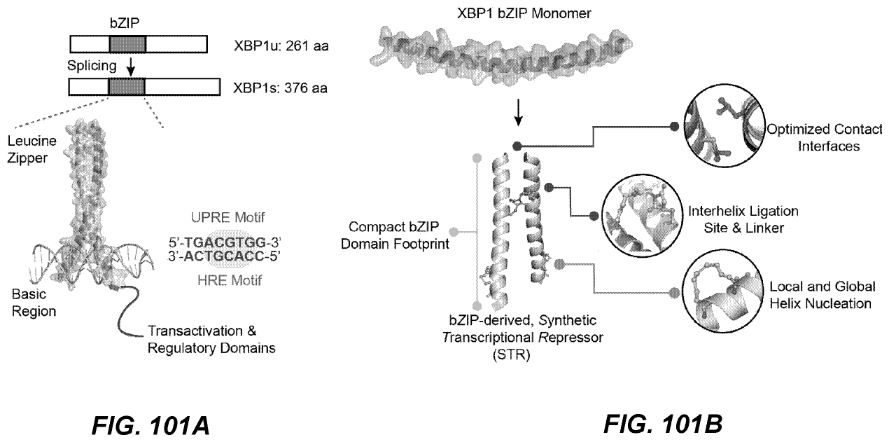

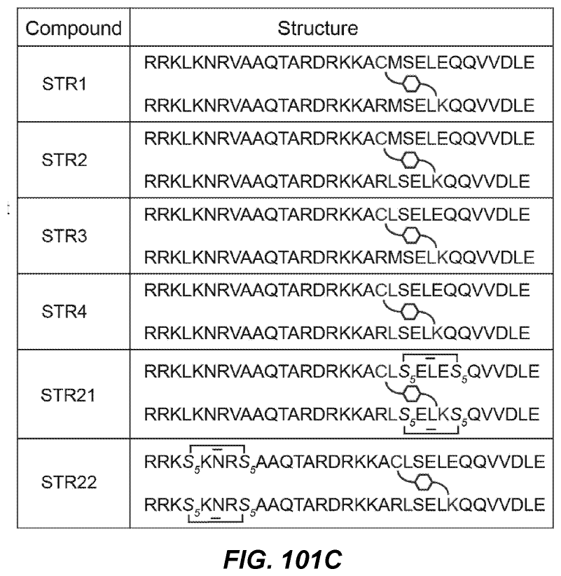

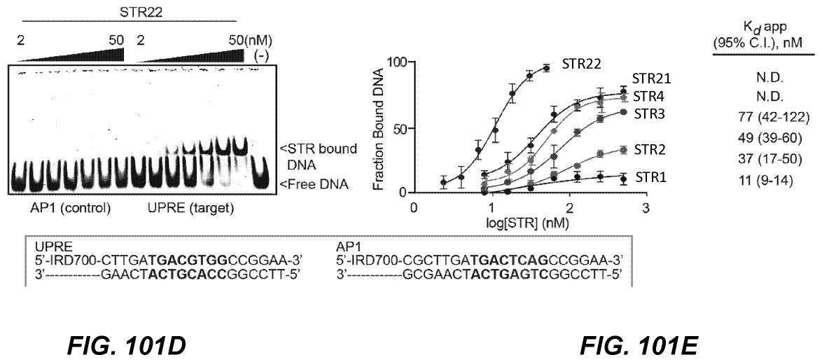

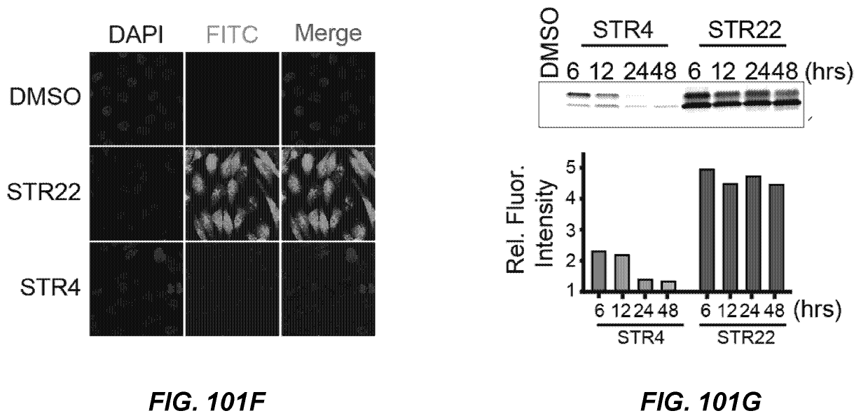

Despite long-standing validation of hypoxia- and HIF1α-induced gene expression as a driver of oncogenic phenotypes, the ability to target this axis has remained elusive. Certain aspects present several critical insights into the development of a new class of molecules capable of directly regulating DNA binding by bZIP TFs, as well as their application in targeting XBP1/HIF1α. First, the inventors developed a modular, convergent synthetic route to create potent and specific ‘synthetic biologic’ STRs that mimic the DNA binding domain of the active, spliced XBP1 homodimer. The inventors showed that stabilization of secondary and tertiary structural elements, identification of a core helical footprint within the parent bZIP TF, and alteration of interfacial contacts are necessary to create STRs with suitable biochemical and pharmacologic properties for DNA binding in cells. It was intriguing to see that dimerized helices from the native XBP1s sequence do not strongly bind DNA, but that mutating two interfacial hydrophobic residues within the nascent leucine zipper core yields molecules that are potent DNA binders (FIG. 101). Subsequent introduction of optimal secondary structure stabilization further improves affinity and specificity, as with STR22, and significantly increases the stability of STRs in cellular environments. While there remains room for the exploration and improvement of the STRs presented here, these data confirm that emergent properties can be realized through vigilant and layered structural stabilization of bZIP STRs, which is in agreement with recent studies of bHLH-derived STRs25. By comparison, TF mimetics consisting of large, natural polypeptide sequences alone pose nontrivial challenges in synthetic tractability and pharmacologic suitability. Perhaps most excitingly, the design principles developed here should be general to most or perhaps all bZIP TFs. This supposition is supported by the fact that no structures of XBP1s exist, and yet the inventors were able to design STR22 from homology models of other bZIP TFs.

Optimized STRs, such as STR22, should prove to be valuable chemical probes to interrogate TF-DNA binding and transcriptional regulation, as well as prototype therapeutics. Here, the inventors reasoned that an XBP1s-derived STR may be capable of targeting both UPRE- and HRE-DNA binding sites within cells due to the embedded HRE motif in the former sequence. The targeted and global ChIP and gene expression profiling studies presented here confirmed this hypothesis and raise intriguing questions about the normal and pathophysiologic crosstalk between these two TFs and their target gene networks. Most importantly, these data confirm that STR22 directly blocks HIF1α binding to target HRE sites in the genome and specifically downregulates hypoxia-induced gene expression programs. This supports the notion that STRs can oppose the action of multiple oncogenic TFs by directly preventing DNA binding, which represents a mechanistically unique and potentially more powerful approach to attenuate pathologic gene expression in diseases like cancer. Along these lines, the recent approval of a small molecule inhibitor of HIF2α⁴⁴ in renal cancer underscores the therapeutic potential in targeting a genetically activated TF (due to VHL inactivation), and raises intriguing questions about the potential to target the expression programs regulated by multiple stress-responsive TFs, such as HIF1α, HIF2α and XBP1s, simultaneously with STRs. Furthermore, the results add mechanistic support for and provide additional therapeutic relevance to previous work implicating the co-regulation of HRE genes by XBP1s and HIF1α in TNBC¹⁵.

### I. Engineered Peptides and DNA Binding Dimers

### A. Engineered Peptides

Aspects of the present disclosure are directed to certain engineered peptides, including engineered bZIP peptides, as well as methods for making and using such engineered peptides, for example in the generation of DNA binding dimers. As used herein, an “engineered bZIP peptide” describes any peptide comprising an amino acid sequence from a portion of a bZIP protein. An engineered bZIP peptide may comprise an unmodified sequence of a region of a bZIP protein. In some aspects, an engineered bZIP peptide of the disclosure comprises a modified basic domain sequence from a bZIP protein. In some aspects, an engineered bZIP peptide of the disclosure comprises a modified leucine zipper domain sequence from a bZIP protein. In some cases an engineered bZIP peptide is a synthetic peptide having one or more modifications relative to a natural bZIP protein. For example, an engineered bZIP peptide may comprise a sequence having 1, 2, 3, 4, 5, 6, 7, 8, 9, 10, 11, 12, 13, 14, 15, 16, 17, 18, 19, 20, 21, 22, 23, 24, 25, or more amino acid substitutions at any position relative to a natural bZIP protein sequence. Additionally or alternatively, an engineered bZIP peptide may comprise 1, 2, 3, 4, 5, 6, 7, 8, 9, 10, or more chemical modifications. For example, an engineered bZIP peptide may comprise one or more modified amino acids (e.g., Lys(Mtt)), one or more non-natural amino acids (e.g., (S)-2-(4′-pentenyl)alanine), one or more intramolecular helix stabilizing linkers, one or more intrapeptide stabilizing linkages, one or more protecting groups, and/or other chemical modifications.

As used herein, a “modified basic domain sequence” of a bZIP protein describes an amino acid sequence which is modified in some way as compared to the natural sequence of the basic domain of the bZIP protein. Thus, for example, a “modified basic domain sequence” of the bZIP protein XBP1 is a sequence having one or more modifications as compared to the natural basic domain sequence of XBP1. Such modifications include, for example, removal of amino acids, amino acid substitutions (including substitutions with non-natural amino acids), and amino acid chemical modifications. In one example, a modified basic domain sequence of a bZIP protein is a sequence that is a portion of the natural basic domain sequence of the bZIP protein but does not comprise the full basic domain sequence. In another example, a modified basic domain sequence of a bZIP protein is a sequence having at least one amino acid substitution (e.g., 1, 2, 3, 4, 5, 6, 7, 8, 9, 10, 11, 12, 13, 14, 15, 16, 17, 18, 19, 20, or more substitutions) relative to the natural basic domain sequence of the bZIP protein. An amino acid substitution may be, for example, substitution for a different natural amino acid, substitution for a modified amino acid (e.g., Lys(Mtt)), or substitution for a non-natural amino acid (e.g., (S)-2-(4′-pentenyl)alanine). For example, where a natural basic domain for a human c-Fos protein has sequence MKRRIRRERNKMAAAKCRNRRREL (SEQ ID NO:108), a modified basic domain sequence of human c-Fos may be IRRERNKMAAAKSRNRRREC (SEQ ID NO:16). Additional example modified basic domain sequences of c-Fos include IRR#RNK#AAAKSRNRRREC (SEQ ID NO:17), EEKRRIRRERNKMAAAKSRNRRREC (SEQ ID NO:18), and EEKRRIRR#RNK#AAAKSRNRRREC (SEQ ID NO:19).

Example modified basic domain sequences are provided in Table 1, below.

TABLE 1

| | | bZIP protein |
|---|---|---|
| | SEQ ID | (from which modified DNA |
| Modified basic domain sequence | NO | binding sequence was derived) |
| IRRERNKMAAAKCRNRRREL | 1 | c-Fos |
| IRRERNKMAAAKSRNRRREC | 16 | c-Fos |
| IRR#RNK#AAAKSRNRRREC | 17 | c-Fos |
| EEKRRIRRERNKMAAAKSRNRRREC | 18 | c-Fos |
| EEKRRIRR#RNK#AAAKSRNRRREC | 19 | c-Fos |
| RKRMRNRIAASKCRKRKLER | 4 | c-Jun |
| RKRMRNRIAASKSRKRKLER | 27 | c-Jun |
| RKR#RNR#AASKSRKRKLER | 28 | c-Jun |
| RIKAERKRMRNRIAASKSRKRKLER | 29 | c-Jun |
| RIKAERKR#RNR#AASKSRKRKLER | 30 | c-Jun |
| RRKLKNRVAAQTARDRKKAR | 7 | XBP1 |
| RRKLKNRVAAQTARDRKKAC | 38 | XBP1 |
| RRK#KNR#AAQTARDRKKAC | 39 | XBP1 |
| RRKLKNRVAAQTARDRKKAR | 43 | XBP1 |
| RRK#KNR#AAQTARDRKKAR | 44 | XBP1 |
| KKMEQNKTAATRYRQKKRAE | 10 | ATF4 |
| KKMEQNKTAATRYRQKKRAC | 48 | ATF4 |
| KKMEQNKTAATRYRQKKRAE | 55 | ATF4 |
| IRRERNNIAVRKSRDKAKMR | 13 | C/EBPβ |
| IRRERNNIAVRKSRDKAKMC | 52 | C/EBPβ |

# is (S)-2-(4′-pentenyl)alanine or
# are intrapeptide stabilizing linkage sites which together form the structure

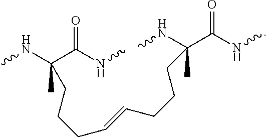

As used herein, a “modified leucine zipper domain sequence” of a bZIP protein describes an amino acid sequence which is modified in some way as compared to the natural sequence of the leucine zipper domain of the bZIP protein. Thus, for example, a “modified leucine zipper domain sequence” of the bZIP protein XBP1 is a sequence having one or more modifications as compared to the natural leucine zipper domain sequence of XBP1. Such modifications include, for example, removal of amino acids, amino acid substitutions (including substitutions with non-natural amino acids), and amino acid chemical modifications. In one example, a modified leucine zipper domain sequence of a bZIP protein is a sequence that is a portion of the natural leucine zipper domain sequence of the bZIP protein but does not comprise the full leucine zipper domain sequence of the bZIP protein. In another example, a modified leucine zipper domain sequence of a bZIP protein comprises a sequence having at least one amino acid substitution (e.g., 1, 2, 3, 4, 5, 6, 7, 8, 9, 10, 11, 12, 13, 14, 15, 16, 17, 18, 19, 20, or more substitutions) relative to the natural leucine zipper domain sequence of the bZIP protein. An amino acid substitution may be, for example, substitution for a different natural amino acid, substitution for a modified amino acid (e.g., Lys(Mtt)), or substitution for a non-natural amino acid (e.g., (S)-2-(4′-pentenyl)alanine). For example, where a natural leucine zipper domain of human c-Fos has sequence TDTLQAETDQLEDEKSALQTEIANLLKEKEKLEFILAAH (SEQ ID NO:109), a modified leucine zipper domain sequence ofhuman c-Fos may be TDTLEDETDQLE (SEQ ID NO:20). Additional example modified leucine zipper domain sequences of human c-Fos include LDELQAEIEQLE (SEQ ID NO:21), IDELQAEIEQLE (SEQ ID NO:22), IDEIQAEIEQIE (SEQ ID NO:23), L#ELQ#EIEQLE (SEQ ID NO:24), I#ELQ#EIEQLE (SEQ ID NO:25), and I#EIQ#EIEQIE (SEQ ID NO:26).

Example modified leucine zipper domain sequences are provided in Table 2, below.

TABLE 2

| | | bZIP protein |
|---|---|---|
| Modified | | (from which modified |
| leucine zipper | SEQ | leucine zipper domain |
| domain sequence | ID NO | sequence was derived) |
| TDTLQAETDQLE | 2 | c-Fos |
| TDTLEDETDQLE | 20 | c-Fos |
| LDELQAEIEQLE | 21 | c-Fos |
| IDELQAEIEQLE | 22 | c-Fos |
| IDEIQAEIEQIE | 23 | c-Fos |
| L#ELQ#EIEQLE | 24 | c-Fos |
| I#ELQ#EIEQLE | 25 | c-Fos |
| I#EIQ#EIEQIE | 26 | c-Fos |
| IARLEEKVKTLK | 5 | c-Jun |
| IA*LEEKVKTLK | 31 | c-Jun |
| IARL*EKVKTLK | 32 | c-Jun |
| AAEL*EKVATLK | 33 | c-Jun |
| IARL*EKIKTLK | 34 | c-Jun |
| IARI*EKIKTIK | 35 | c-Jun |
| I#RL*#KVKTLK | 36 | c-Jun |
| I#RL*#KIKTLK | 37 | c-Jun |
| MSELEQQVVDLE | 8 | XBP1 |
| MSELEQQVVDLE | 40 | XBP1 |
| LSELEQQVVDLE | 41 | XBP1 |
| L#ELE#QVVDLE | 42 | XBP1 |
| MSEL*QQVVDLE | 45 | XBP1 |
| LSEL*QQVVDLE | 46 | XBP1 |
| L#EL*#QVVDLE | 47 | XBP1 |
| QEALTGECKELE | 11 | ATF4 |
| QEALTGELKELE | 49 | ATF4 |
| LEALKAELKELR | 50 | ATF4 |
| L#ALK#ELKELR | 51 | ATF4 |
| QEAL*GELKELE | 56 | ATF4 |
| LEAL*AELKELR | 57 | ATF4 |
| L#AL*#ELKELR | 58 | ATF4 |
| NLETQHKVLELT | 14 | C/EBPB |
| LLELQHKVLELR | 53 | C/EBPB |
| L#ELQ#KVLELR | 54 | C/EBPB |

Example engineered bZJP peptides contemplated herein and useful in compositions and methods of the present disclosure are provided in Table 3, below. Additional engineered bZJP peptides beyond those listed in Table 3 are contemplated herein.

TABLE 3

| Engineered bZIP Peptide | SEQ ID NO |
|---|---|
| Ac-IRRERNKMAAAKSRNRRRECTDTLEDETDQLE-NH₂ | 59 |
| Ac-IRRERNKMAAAKSRNRRRECLDELQAEIEQLE-NH₂ | 60 |
| Ac-IRRERNKMAAAKSRNRRRECIDELQAEIEQLE-NH₂ | 61 |
| Ac-IRRERNKMAAAKSRNRRRECIDEIQAEIEQIE-NH₂ | 62 |
| Ac-IRR#RNK#AAAKSRNRRRECLDELQAEIEQLE-NH₂ | 63 |
| Ac-IRRERNKMAAAKSRNRRRECL#ELQ#EIEQLE-NH₂ | 64 |
| Ac-IRR#RNK#AAAKSRNRRRECIDELQAEIEQLE-NH₂ | 65 |
| Ac-IRRERNKMAAAKSRNRRRECI#ELQ#EIEQLE-NH₂ | 66 |
| Ac-IRR#RNK#AAAKSRNRRRECIDEIQAEIEQIE-NH₂ | 67 |
| Ac-IRRERNKMAAAKSRNRRRECI#EIQ#EIEQIE-NH₂ | 68 |
| Ac-EEKRRIRRERNKMAAAKSRNRRRECLDELQAEIEQLE-NH₂ | 69 |
| Ac-EEKRRIRR#RNK#AAAKSRNRRRECLDELQAEIEQLE-NH₂ | 70 |
| Ac-EEKRRIRRERNKMAAAKSRNRRRECL#ELQ#EIEQLE-NH₂ | 71 |
| Ac-RKRMRNRIAASKSRKRKLERIAKₘLEEKVKTLK-NH₂ | 72 |
| Ac-RKRMRNRIAASKSRKRKLERIARLKₘEKVKTLK-NH₂ | 73 |
| Ac-RKRMRNRIAASKSRKRKLERAAELKₘEKVATLK-NH₂ | 74 |
| Ac-RKRMRNRIAASKSRKRKLERIARLKₘEKIKTLK-NH₂ | 75 |
| Ac-RKRMRNRIAASKSRKRKLERIARIKₘEKIKTIK-NH₂ ( | 76 |
| Ac-RKR#RNR#AASKSRKRKLERIARLKₘEKVKTLK-NH₂ | 77 |
| Ac-RKRMRNRIAASKSRKRKLERI#RLKₘ#KVKTLK-NH₂ | 78 |
| Ac-RKR#RNR#AASKSRKRKLERIARLKₘEKIKTLK-NH₂ | 79 |
| Ac-RKRMRNRIAASKSRKRKLERI#RLKₘ#KIKTLK-NH₂ | 80 |
| Ac-RIKAERKRMRNRIAASKSRKRKLERIARLKₘEKVKTLK-NH₂ | 81 |
| Ac-RIKAERKR#RNR#AASKSRKRKLERIARLKₘEKVKTLK-NH₂ | 82 |
| Ac-RIKAERKRMRNRIAASKSRKRKLERI#RLKₘ#KVKTLK-NH₂ | 83 |
| Ac-RRKLKNRVAAQTARDRKKACMSELEQQVVDLE-NH₂ | 84 |
| Ac-RRKLKNRVAAQTARDRKKACLSELEQQVVDLE-NH₂ | 85 |
| Ac-RRKLKNRVAAQTARDRKKACL#ELE#QVVDLE-NH₂ | 86 |
| Ac-RRK#KNR#AAQTARDRKKACLSELEQQVVDLE-NH₂ | 87 |
| Ac-RRKLKNRVAAQTARDRKKARMSELKₘQQVVDLE-NH₂ | 88 |
| Ac-RRKLKNRVAAQTARDRKKARLSELKₘQQVVDLE-NH₂ | 89 |
| Ac-RRKLKNRVAAQTARDRKKARL#ELKₘ#QVVDLE-NH₂ | 90 |
| Ac-RRK#KNR#AAQTARDRKKARLSELKₘQQVVDLE-NH₂ | 91 |
| Ac-KKMEQNKTAATRYRQKKRACQEALTGELKELE-NH₂ | 92 |
| Ac-KKMEQNKTAATRYRQKKRACLEALKAELKELR-NH₂ | 93 |
| Ac-KKMEQNKTAATRYRQKKRACL#ALK#ELKELR-NH₂ | 94 |
| Ac-KKMEQNKTAATRYRQKKRAEQEALKₘGELKELE-NH₂ | 95 |
| Ac-KKMEQNKTAATRYRQKKRAELEALKₘAELKELR-NH₂ | 96 |
| Ac-KKMEQNKTAATRYRQKKRAEL#ALKₘ#ELKELR-NH₂ | 97 |
| Ac-IRRERNNIAVRKSRDKAKMCLLELQHKVLELR-NH₂ | 98 |
| Ac-IRRERNNIAVRKSRDKAKMCL#ELQ#KVLELR-NH₂ | 99 |

Ac is acetyl; # is (S)-2-(4'-pentenyl)alanine; Kₘ is Lys(Mmt) or a Lys residue linked to a maleimide linker

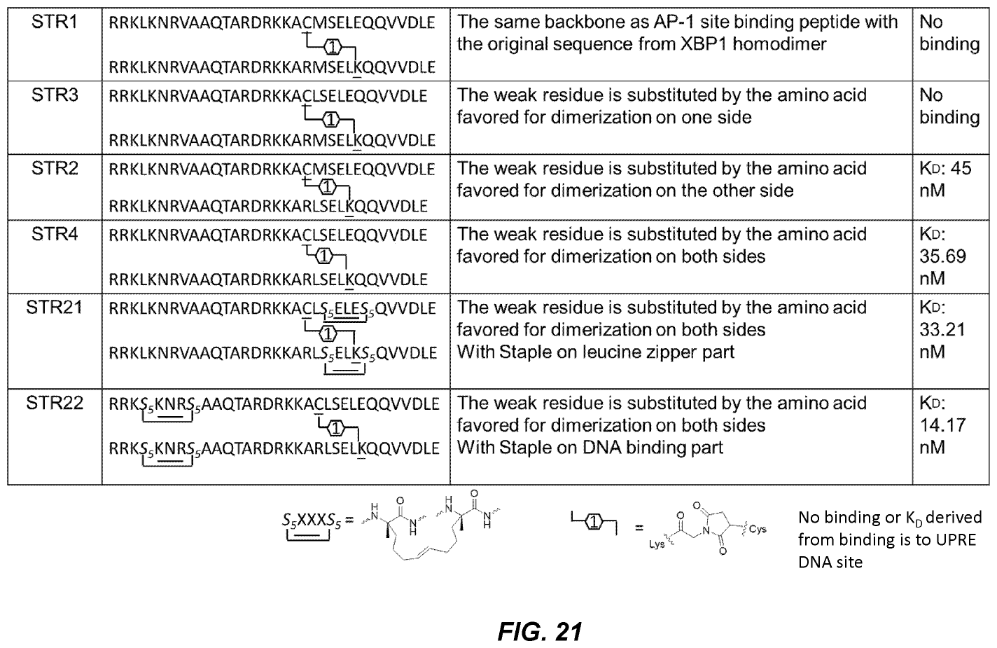

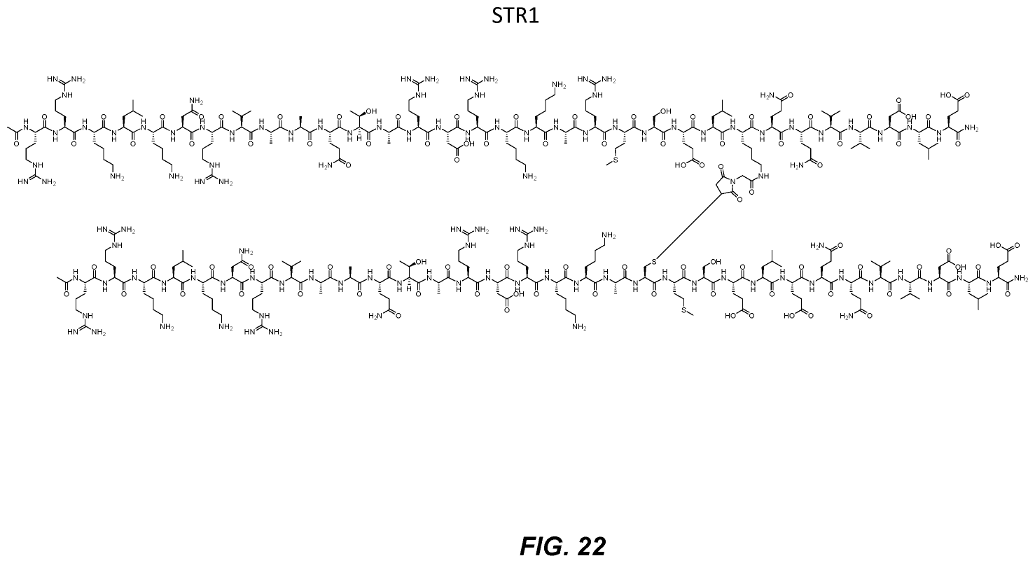

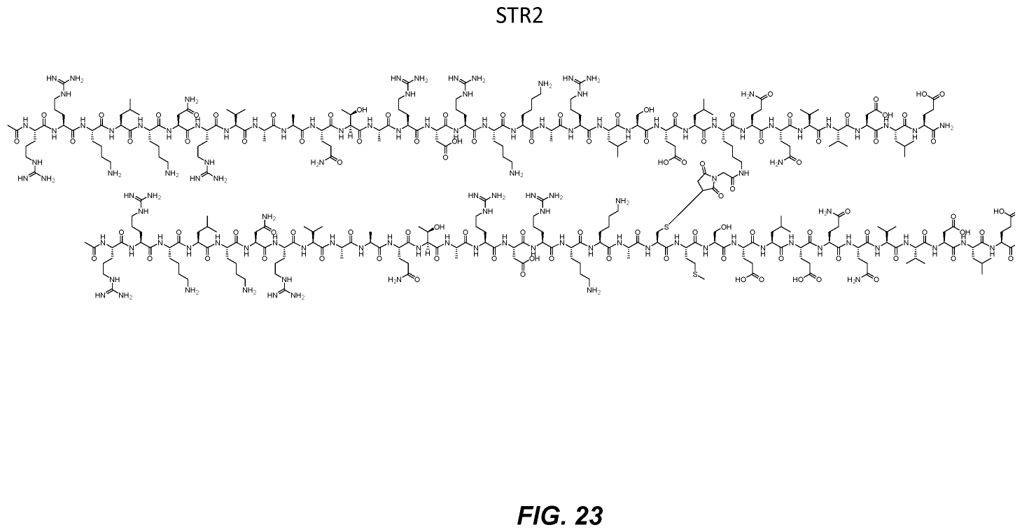

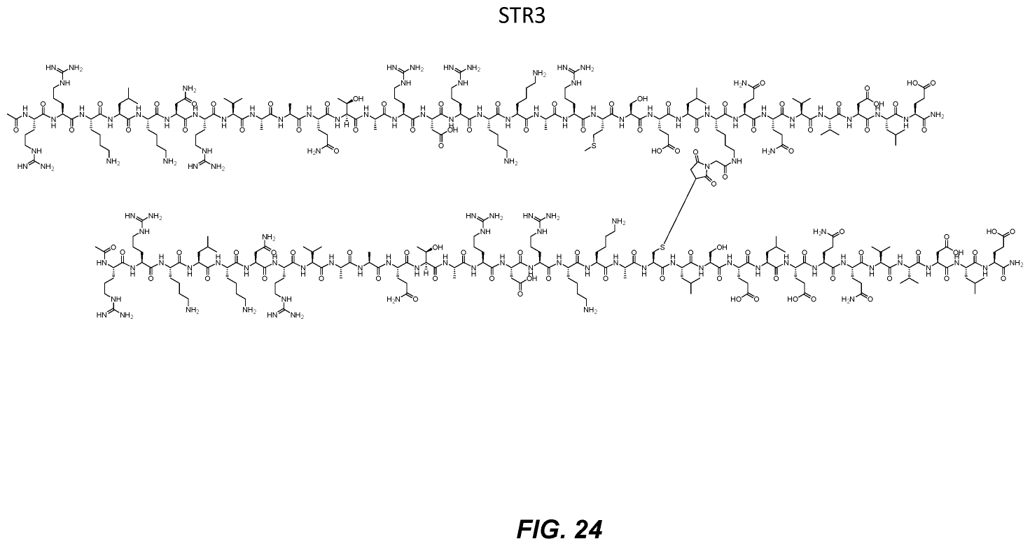

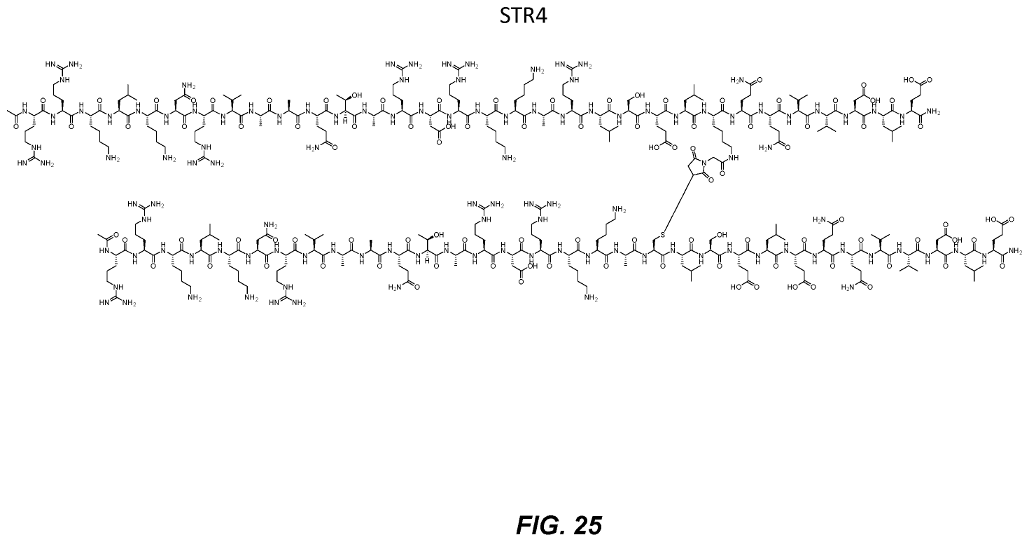

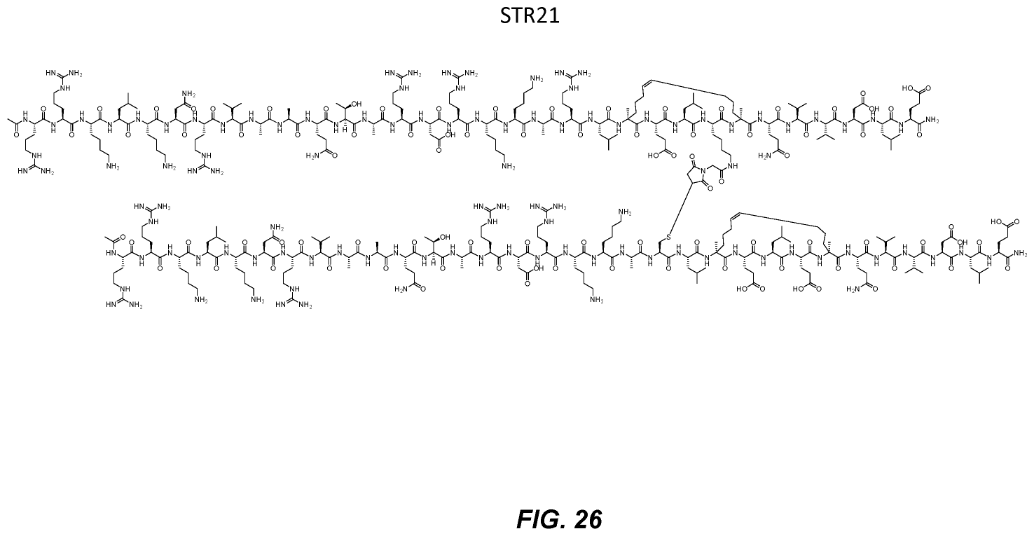

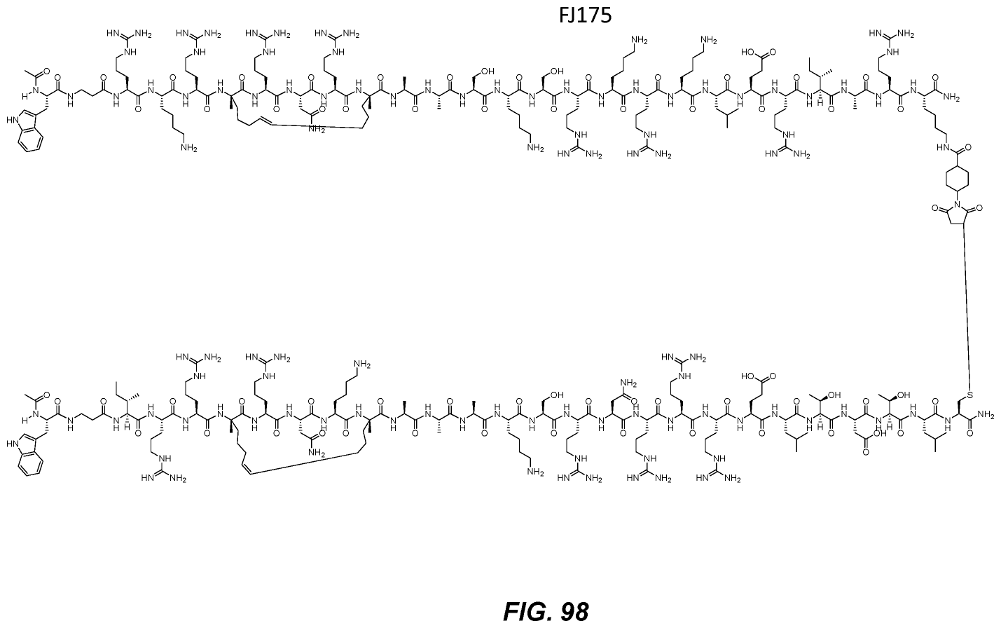

FIGS. 21-98 show certain example synthetic dimers generated from engineered bZIP peptides.

### B. DNA Binding Dimers

Aspects of the present disclosure are directed to certain engineered DNA-binding dimers, including those generated from engineered bZIP peptides, along with methods of making and using such DNA-binding dimers. As used herein, an “engineered DNA-binding dimer,” describes a molecule comprising two engineered peptides linked together via a covalent linkage, where said molecule is capable of binding to DNA. In some cases, an engineered DNA-binding dimer of the disclosure comprises two engineered bZIP peptides linked via an interpeptide linkage; in such cases the engineered DNA-binding dimer is also referred to herein as a “bZIP transcriptional repressor,” a “synthetic transcriptional repressor” or an “STR”. In some cases, an interpeptide linkage of the disclosure is a side-by-side interpeptide linkage. As used herein, a “side-by-side interpeptide linkage” (also “side-by-side linkage”) describes a covalent, chemical linkage between two peptides (including between two synthetic or engineered peptides), where the linkage is between a first amino acid (including a natural amino acid, modified amino acid, or non-natural amino acid) of a first peptide and a second amino acid of a second peptide, where the first amino acid is located at an interior of the first peptide and the second amino acid is located at an interior of the second peptide. By way of example, the interior of a peptide comprises the non-terminal amino acids of the peptide, such that the linkage between the first and second peptide is between one or more non-terminal amino acids (i.e. an amino acid not comprising a C-terminal or N-terminal amino acid). Therefore, in certain aspects, a “side-by-side interpeptide linkage”, as used herein, does not include a linkage between a first and second peptide where the linkage is between one or more terminal (C-terminal or N-terminal) amino acids.

### 1. Interpeptide Linkages

A bZIP transcriptional repressor of the disclosure is capable of binding to a bZIP protein binding site on DNA, as well as of competing with a natural (also “native”) bZIP protein for binding to the binding site. An interpeptide linkage may be any chemical linkage that covalently attaches two polypeptides (e.g., engineered bZIP peptides). In some aspects, at least one of the peptides comprise one or two (or more) linker residues. Linker residues may be natural (e.g., cysteine) or unnatural (e.g., displaing a thiol, azide, maleimide, alkyne, etc.) amino acids that facilitate the formation of linkages (e.g., covalent linkages) between the peptide and a second peptide comprising complementary linker residues. In some aspects, peptides comprise a first linker residue at the N terminal residue (e.g., azide or alkyne). In some aspects, the peptide comprises a linker residue (e.g., thiol of maleimide) at a position 1 to 25 (e.g., 1, 2, 3, 4, 5, 6, 7, 8, 9, 10, 11, 12, 13, 14, 15, 16, 17, 18, 19, 20, 21, 22, 23, 24, 25, or ranges therebetween) amino acids from the N-terminus. In some aspects, the peptides comprise more than one linker. In some aspects, the peptides comprise a linker at the N terminal residue and at least one additional linker residues at a position 1 to 25 (e.g., 1, 2, 3, 4, 5, 6, 7, 8, 9, 10, 11, 12, 13, 14, 15, 16, 17, 18, 19, 20, 21, 22, 23, 24, 25, or ranges therebetween) amino acids from the N-terminus. In some aspects, the peptide comprises at least two linker residues at a position 1 to 25 (e.g., 1, 2, 3, 4, 5, 6, 7, 8, 9, 10, 11, 12, 13, 14, 15, 16, 17, 18, 19, 20, 21, 22, 23, 24, 25, or ranges therebetween) amino acids from the N-terminus. In some aspects, hydrocarbon stabling, such as the stapling described herein, within the alpha helix stabilizes the alpha helix, while linkage of the two peptides together (e.g., at two positons) provides proper (e.g., optimized) orientation of the two peptides (e.g., with respect to a DNA binding site).

Various chemical linkages are recognized in the art and contemplated herein. Example interpeptide linkages of the present disclosure include

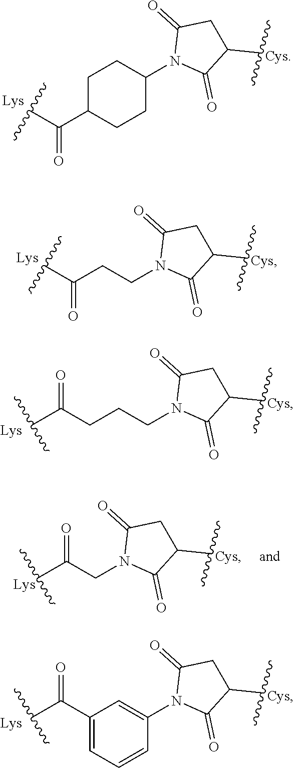

where “Cys” represents a cysteine residue (natural or modified) on a first engineered bZIP peptide and “Lys” represents a lysine residue (natural or modified) on a second engineered bZIP peptide. In some aspects, the interpeptide linkage is

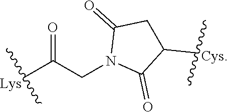 [SMILES: *C(=O)CN1C(=O)CC(*)C1=O]

Interpeptide linkages contemplated herein include those described in, for example, U.S. Patent Application Publication 2019/0135868, incorporated herein by reference.

In some aspects, a linker residue is a natural or unnatural amino acid that, which may include

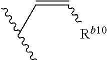

each instance of M₁ and M is independently optionally substituted alkylene; optionally substituted alkenylene; cyclic or acyclic, optionally substituted alkynylene; optionally substituted heteroalkylene; optionally substituted heteroalkenylene; optionally substituted heteroalkynylene; optionally substituted arylene; or optionally substituted heteroarylene;
each instance of R^{b1} and R^{b2} is independently selected from the group consisting of each hydrogen; optionally substituted aliphatic; optionally substituted heteroaliphatic; optionally substituted aryl; optionally substituted heteroaryl;

Nu is —SH—, —OH—, —NHR^{b5}, —NH—NHR^{b5}, —N═NH, —N=C, —N₃, or

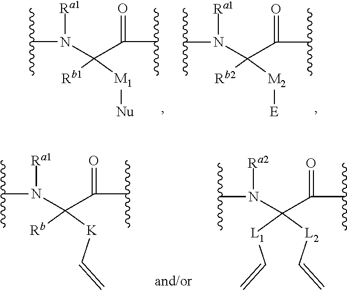

wherein
wherein R^{b10} is hydrogen, optionally substituted aliphatic, or optionally substituted heteroaliphatic; and R^{b5} is hydrogen, optionally substituted aliphatic, optionally substituted heteroaliphatic, or an amino protecting group;
E is a leaving group, which may comprise, CHO, CO2R^{b6}, COX^{b7},

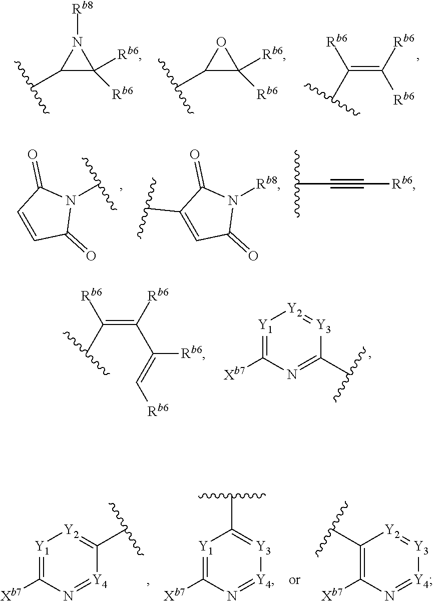

wherein: R^{b6} is hydrogen, optionally substituted aliphatic, or optionally substituted heteroaliphatic, or wherein two R^{b6} groups are joined to form an optionally substituted carbocyclic or optionally substituted heterocyclic ring;
X^{b7} is a leaving group;
each instance of Y₁, Y₂, Y₃, and Y₄ is independently selected from N or C(R^{b6})—;
R^{b8} is hydrogen, optionally substituted aliphatic, optionally substituted heteroaliphatic, optionally substituted aryl, optionally substituted heteroaryl, or an amino protecting group;
each instance of K, L₁, and L₂, is, independently, optionally substituted alkylene; optionally substituted heteroalkylene; optionally substituted arylene; or optionally substituted heteroarylene;
each instance of R^{a1} and R^{a2} is, independently, hydrogen; optionally substituted aliphatic; optionally substituted heteroaliphatic; optionally substituted aryl; optionally substituted heteroaryl; acyl; or an amino protecting group; and
each instance of R^{b} is, independently, hydrogen; optionally substituted aliphatic; optionally substituted heteroaliphatic; optionally substituted aryl; optionally substituted heteroaryl. In some aspects, Nu and E are joined to form a conjugated group comprising one of

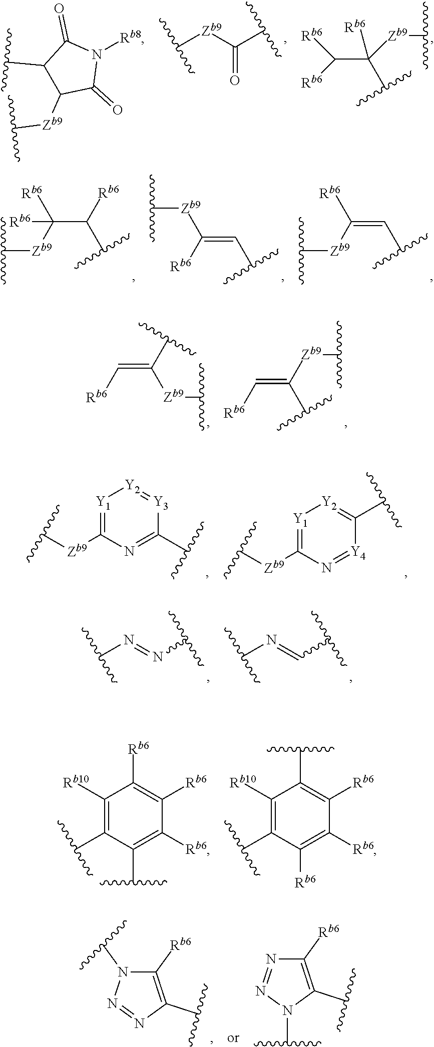

wherein Z^{b9} is —S—, —O—, N(R^{b5})—, NH—N(R^{b5})—, or N═N—. In certain aspects, R^{b10} is hydrogen. In certain aspects, R^{b6} is hydrogen or optionally substituted aliphatic, e.g., acyl. In some aspects, each instance of Y₁, Y₂, Y₃, and Y₄ is independently selected from N or C(R^{b6}). In certain aspects, Nu is SH and Z^{b9} is —S—. In certain aspects, Nu is OH and Z^{b9} is —O—. In certain aspects, Nu is —NHR^{b}s and Z^{b9} is N(R^{b5})—. In certain aspects, Nu is NH—NHR^{b5} and Z^{b9} is NH—N(R^{b5})—. In certain aspects, Nu is —N═NH and Z^{b9} is —N═N—. In certain aspects, R^{b5} is hydrogen.

In some aspects, one or more linker residue from one peptide are reacted with one or more linker residues from a second peptide to create an interpeptide linkage.

### 2. Intrapeptide Stabilizing Linkages

An engineered DNA-binding dimer may comprise one or more intrapeptide stabilizing linkages. In some aspects, intrapeptide stabilizing linkages can be hydrocarbon staples. “Stapling” as used herein, refers to a process by which two terminally unsaturated amino acid side chains in a polypeptide chain react with each other in the presence of a ring closing metathesis catalyst to generate an intrapeptide stabilizing linkage between the two amino acids. In some aspects, two amino acids (e.g., i and i+4, i and i+7, etc.) within the alpha helical segment of at least one peptide in the DNA-binding dimer are modified to allow an intrapeptide stabilizing linkage between the two amino acids. In some aspects, the intrapeptide stabilizing linkage stabilizes the alpha helix and allows for DNA binding by the peptide in the absence of a larger polypeptide. In some aspects, the intrapeptide stabilizing linkage is between two non-natural amino acids. The non-natural amino acids may be S5, R8, S-2-(4′-pentenyl) alanine, R-2-(7′-octenyl) alanine, (R)—N-Fmoc-2-(7′-octenyl) alanine, and/or (S)—N-Fmoc-2-(4′-pentenyl) alanine. In some aspects, the intrapeptide stabilizing linkage comprises one or more lactam connections, cross-coupling mediated C—C bond connections, thioethers, ethers, secondary or tertiary amines, ketone connections, triazole connections, dials-alder adducts, and/or inverse electron demand diels-alder adducts. In some aspects, the intrapeptide stabilizing linkage comprises chemical reactions between two amino acids. The reactions may include thiol alkylation, thiol/amine alkylation/acylation, dials-alder, [3+2] click chemistry, and/or amide bond formation (macrolactamization) reactions. The peptides may also comprise other helix-stabilizing moieties to increase stability and/or otherwise alter DNA binding. Such moieties may comprise aminoisobutyric acid, D-amino acids and/or other natural or unnatural substitutions.

In some aspects, the peptides comprise one or more occurrences of an intrapeptide stabilizing linkage that include

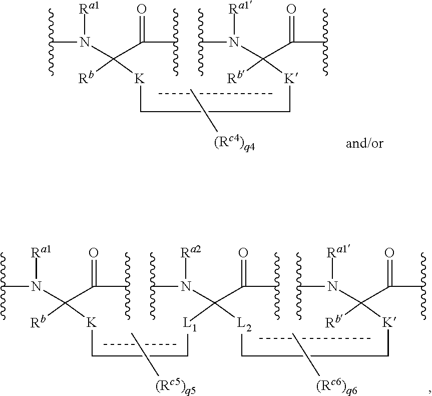

wherein each instance of K, K′, L₁, and L₂, is, independently, optionally substituted alkylene; optionally substituted heteroalkylene; optionally substituted arylene; or optionally substituted heteroarylene;
each instance of R^{a1}, R^{a1′}, and R^{a2} is, independently, hydrogen; optionally substituted aliphatic; optionally substituted heteroaliphatic; optionally substituted aryl;
optionally substituted heteroaryl; acyl; or an amino protecting group;
each instance of R^{b} and R^{b′} is, independently, hydrogen; optionally substituted aliphatic; optionally substituted heteroaliphatic; optionally substituted aryl; optionally substituted heteroaryl;
each instance of [symbol]
   independently represents a single or double bond;

each instance of R^{a1}, R^{c5}, and R^{c6} is independently hydrogen; cyclic or acyclic, branched or unbranched, substituted or unsubstituted aliphatic; cyclic or acyclic, branched or unbranched, substituted or unsubstituted heteroaliphatic; substituted or unsubstituted aryl; substituted or unsubstituted heteroaryl; substituted or unsubstituted acyl; substituted or unsubstituted hydroxyl; substituted or unsubstituted thiol; substituted or unsubstituted amino; azido; cyano; isocyano; halo; or nitro; and
each instance of q^{c4}, q^{c6}, and q^{c6} is independently 0, an integer between 1 and 2 when [symbol]
   represents a double bond, or an integer between 1 and 4 when [symbol]
   represents a single bond.

In some aspects, the peptides comprise one or more occurrences of an intrapeptide stabilizing linkage that include

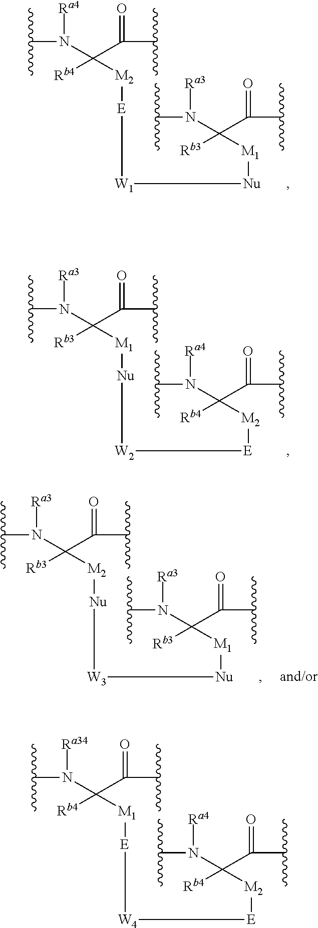

wherein:
each instance of M₁ and M₂ is independently optionally substituted alkylene; optionally substituted alkenylene; cyclic or acyclic, optionally substituted alkynylene; optionally substituted heteroalkylene; optionally substituted heteroalkenylene; optionally substituted heteroalkynylene; optionally substituted arylene; or optionally substituted heteroarylene;
each instance of R^{b3} and R^{b4} is independently selected from the group consisting of each hydrogen; optionally substituted aliphatic; optionally substituted heteroaliphatic; optionally substituted aryl; optionally substituted heteroaryl;
each instance of -Nu-W₁-E- and -Nu-W₂-E- independently represents any one of the following groups:

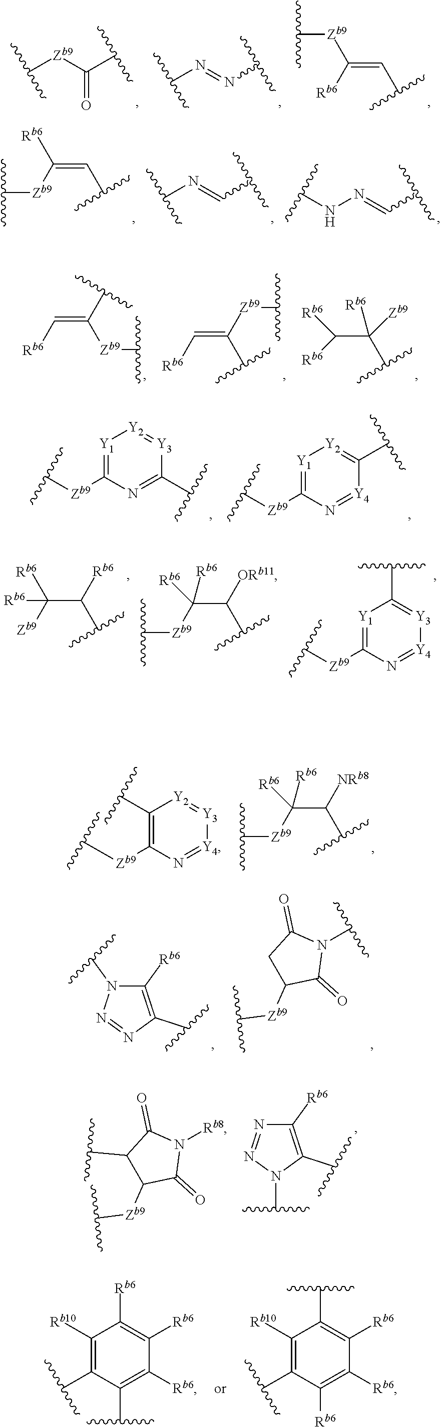

where
Z^{b9} is —O—, —S—, —N(R^{b5})—, —NH—N(R^{b5})—, —N═N—, or —NC—; and R^{b5} is hydrogen, optionally substituted aliphatic, optionally substituted heteroaliphatic, or an amino protecting group;
R^{b6} is hydrogen, optionally substituted aliphatic, or optionally substituted heteroaliphatic, or two R^{b6} groups are joined to form an optionally substituted carbocyclic or optionally substituted heterocyclic ring;
each instance of Y₁, Y₂, Y₃, and Y₄ is independently selected from —N— or —C(R^{b6})—;
R^{b8} is hydrogen, optionally substituted aliphatic, optionally substituted heteroaliphatic, optionally substituted aryl, optionally substituted heteroaryl, or an amino protecting group;
R^{b10} is hydrogen, optionally substituted aliphatic, or optionally substituted heteroaliphatic;
R^{b11} is hydrogen, optionally substituted aliphatic, optionally substituted heteroaliphatic, optionally substituted aryl, optionally substituted heteroaryl, or an oxygen protecting group;
each instance of -Nu-W₃-Nu- independently represents

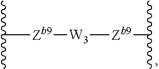

wherein

Z^{b9} is —O—, —S—, —N(R^{b5})—, —NH—N(R^{b6})—, N═N—, or N=C; R^{b5} is hydrogen, optionally substituted aliphatic, optionally substituted heteroaliphatic, or an amino protecting group; and W₃ is selected from the group consisting of optionally substituted alkylene; optionally substituted alkenylene; cyclic or acyclic, optionally substituted alkynylene; optionally substituted heteroalkylene; optionally substituted heteroalkenylene; optionally substituted heteroalkynylene; optionally substituted arylene; or optionally substituted heteroarylene; and

each instance of -E-W₄-E- independently represents optionally substituted alkylene; optionally substituted alkenylene; cyclic or acyclic, optionally substituted alkynylene; optionally substituted heteroalkylene; optionally substituted heteroalkenylene; optionally substituted heteroalkynylene; optionally substituted arylene; or optionally substituted heteroarylene.

An engineered DNA-binding dimer may comprise 1, 2, 3, 4, 5, 6, or more intrapeptide stabilizing linkages. In some cases, an engineered DNA-binding dimer of the disclosure does not comprise any intrapeptide stabilizing linkages. In certain aspects, intrapeptide stabilizing linkages are the result of ring-closing olefin metathesis (RCM) of hindered α-methyl, α-alkenyl amino acids (e.g., (S)-2-(4′-pentenyl)alanine). Various methods for intrapeptide stabilizing linkage are contemplated herein, including, for example, those described in Cromm et al., ACS Chem Biol. 2015; 10(6):1362-1375; Walensky et al., J Med Chem. 2014; 57(15):6275-6288; U.S. Patent Application Publication No. 2019/0135868; and U.S. Pat. No. 10,259,848, all of which are incorporated herein by reference in their entirety.

### 3. Examples of Peptides and Dimers

In some aspects, an engineered DNA-binding dimer of the disclosure has a particular affinity for binding to a region of DNA. In some aspects, the DNA-binding dimer binds to one or more regions of DNA comprising a particular motif. The motif may be a canonical DNA motifs, such as the unfolded protein response element (UPRE) and/or hypoxia-induced response element (HRE). The motif may comprise ACGTG, ACGTGC, ACGTGA, ACGTGT, TACGTG, GACGTG, AACGTG, or DACGTGH (wherein D is T, G, or A and H is A, C, or T). In some aspects, the DNA-binding dimer binding to one or more regions of DNA causes transcriptional repression of one or more genes regulated by the region of DNA. Affinity may be expressed as a dissociation constant (K_{D}). An engineered DNA-binding dimer of the present disclosure may have a K_{D} for binding to a region of DNA of at least, at most, or about 500, 400, 300, 200, 150, 100, 50, 40, 30, 29, 28, 27, 26, 25, 24, 23, 22, 21, 20, 19, 18, 17, 16, 15, 14, 13, 12, 11 10, 9, 8, 7, 6, 5, 4, 3, 2, 1, 0.1, or 0.01 nM, or any range or value derivable therein. In some aspects, the K_{D} is measured for binding ability to a UPRE, AARE, CRE, and/or AP-1 sequence. In some aspects, a synthetic dimer disclosed herein does not bind specifically to DNA. In some aspects, a synthetic dimer disclosed herein does not bind specifically to a UPRE, AARE, CRE, and/or AP-1 sequence. In some aspects, when a synthetic dimer has “no binding” or “no stable binding” to a DNA sequence, the engineered DNA-binding dimer shows no band with defined shape formed when tested by EMSA, and there is only obscure smearing between the top of the gel and free band. In some aspects, where an engineered DNA-binding dimer is a bZIP transcriptional repressor, the bZIP transcriptional repressor has a binding affinity for a bZIP protein target DNA sequence (e.g., UPR element, AP-1 site, etc.) of at most or about 300, 200, 150, 100, 50, 40, 30, 29, 28, 27, 26, 25, 24, 23, 22, 21, 20, 19, 18, 17, 16, 15, 14, 13, 12, 11 10, 9, 8, 7, 6, 5, 4, 3, 2, 1, 0.1, or 0.01 nM, or any range or value derivable therein.

In certain aspects, a peptide disclosed herein comprises a non-natural amino acid. The amino acid may be in an (R) configuration or an (S) configuration. In some aspects, the non-natural amino acid comprises one or more of

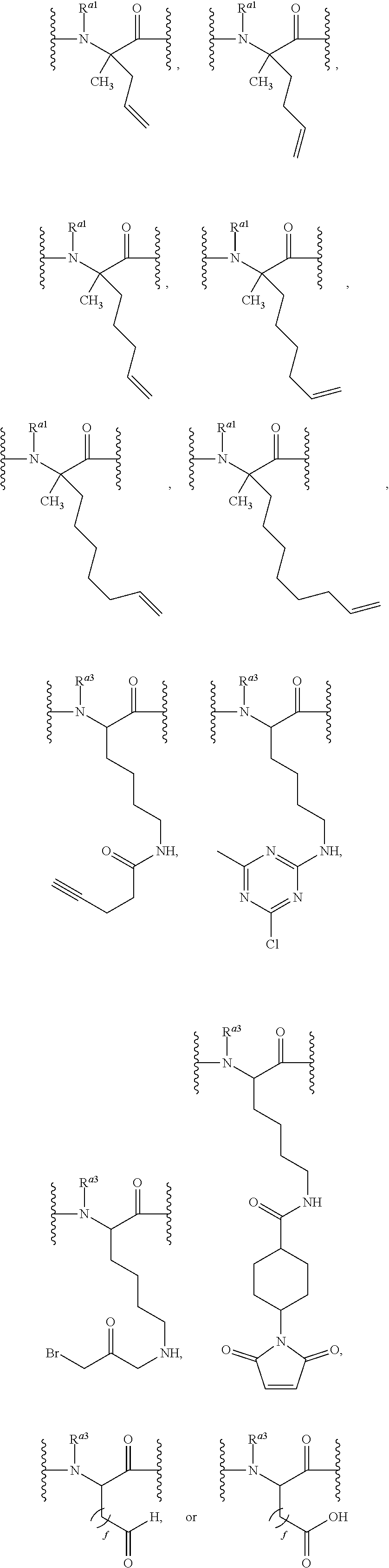

any of which may be in an (R) configuration or an (S) configuration, wherein each instance of R^{a1} and/or R^{a3} is, independently, hydrogen; optionally substituted aliphatic; optionally substituted heteroaliphatic; optionally substituted aryl; optionally substituted heteroaryl; acyl; or an amino protecting group, f is an integer between 1 and 10, inclusive (e.g., f is 1, 2, 3, 4, 5, 6, 7, 8, 9, or 10). In certain aspects, f is 1.

In some aspects, the non-natural amino acid comprises

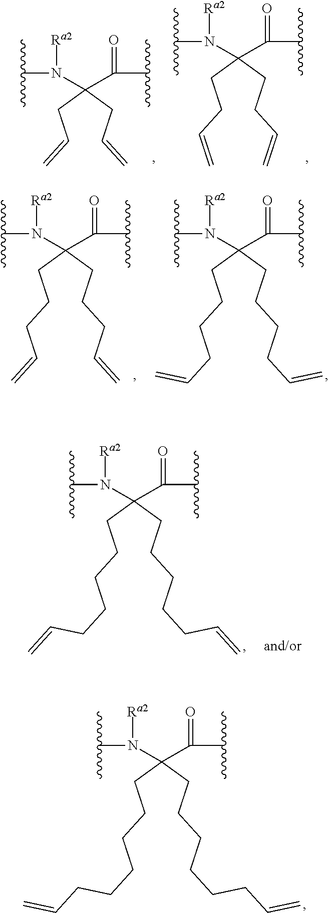

any of which may be in an (R) configuration or an (S) configuration, wherein each instance of R^{a2} is, independently, hydrogen; optionally substituted aliphatic; optionally substituted heteroaliphatic; optionally substituted aryl; optionally substituted heteroaryl; acyl; or an amino protecting group.

Certain examples of bZIP transcriptional repressors of the disclosure are shown in FIGS. 21-98 and provided below:

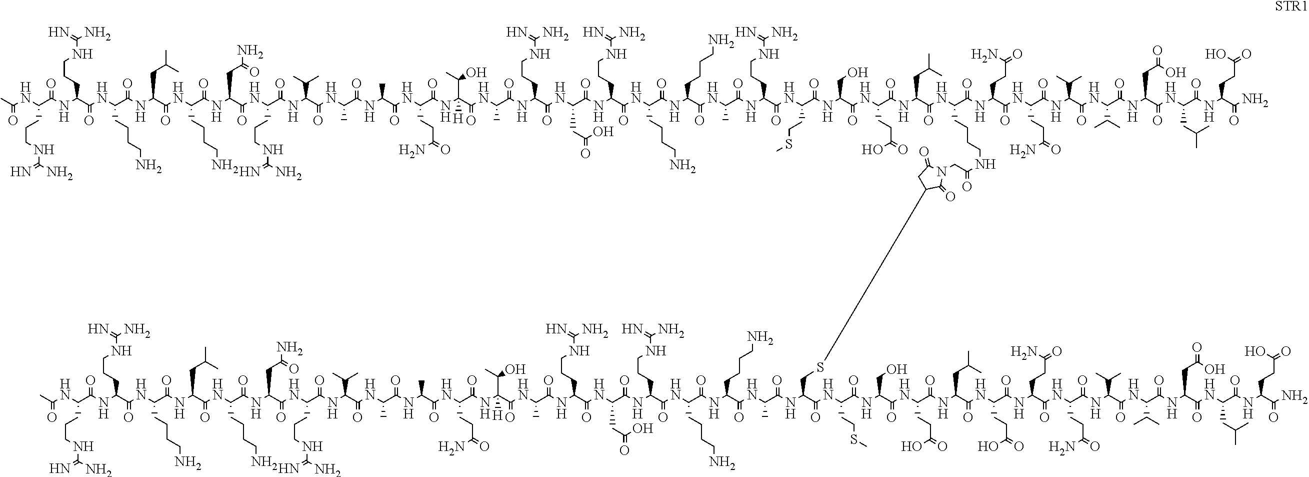

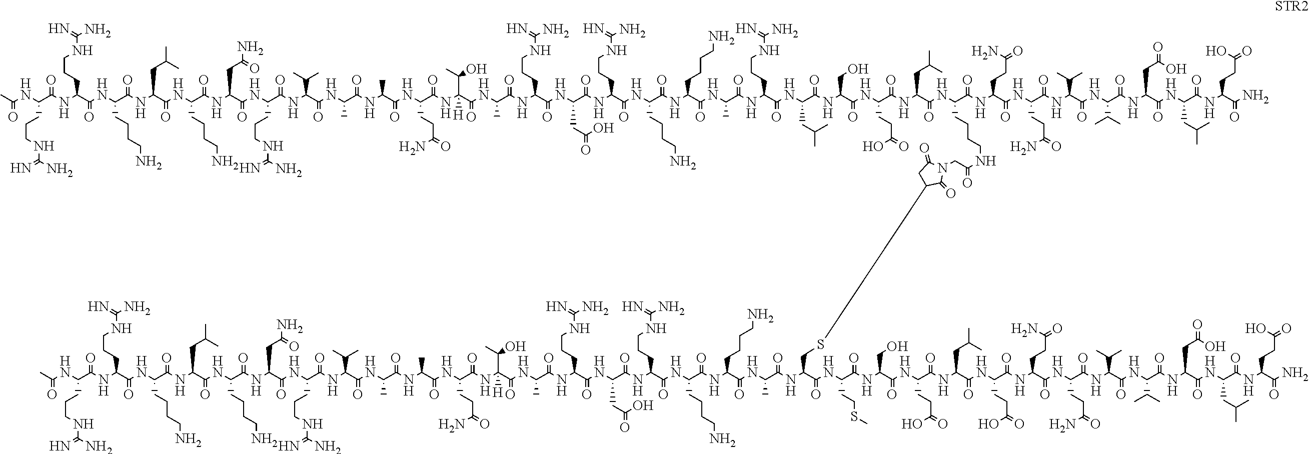

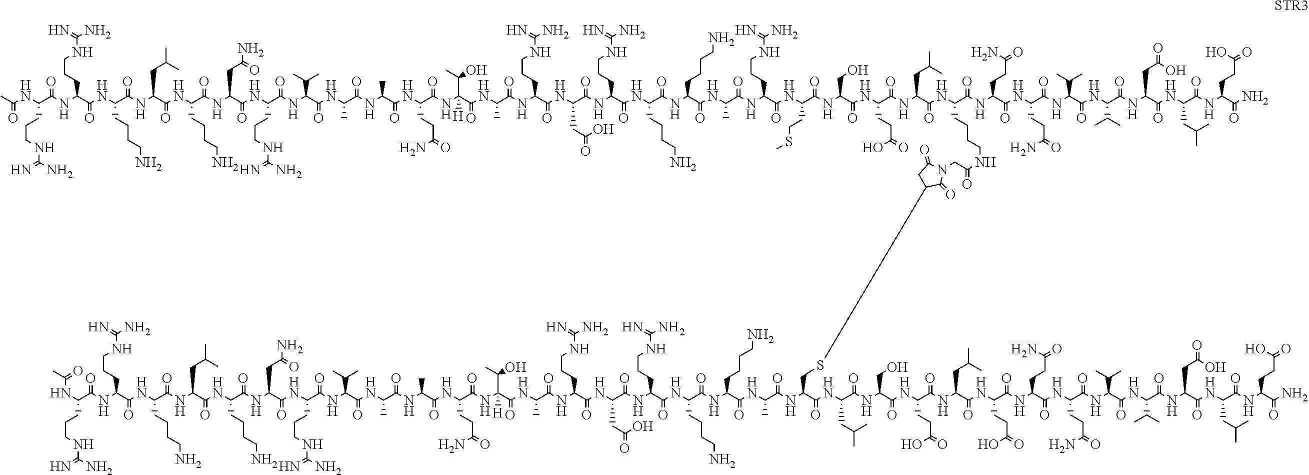

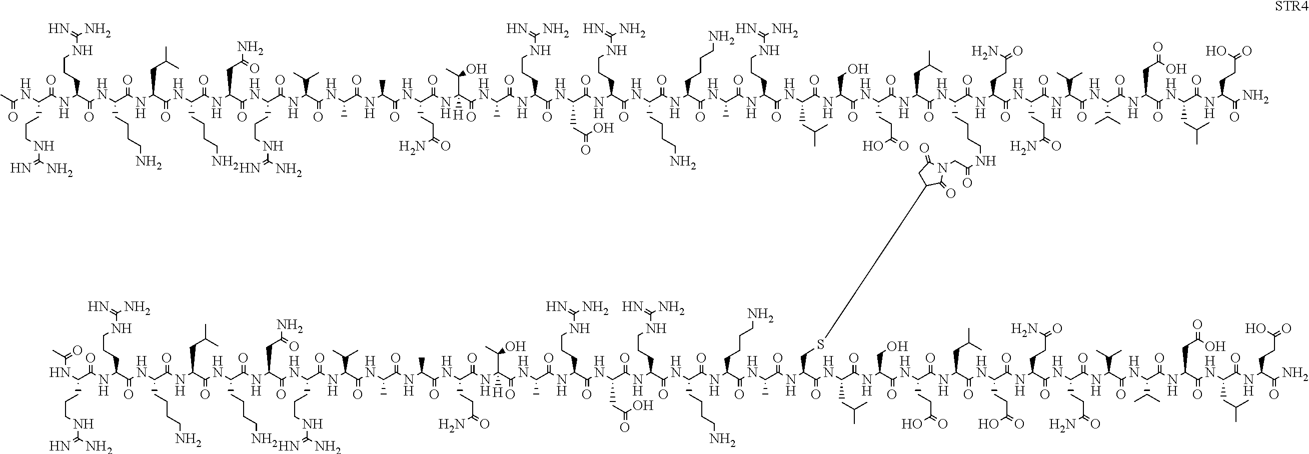

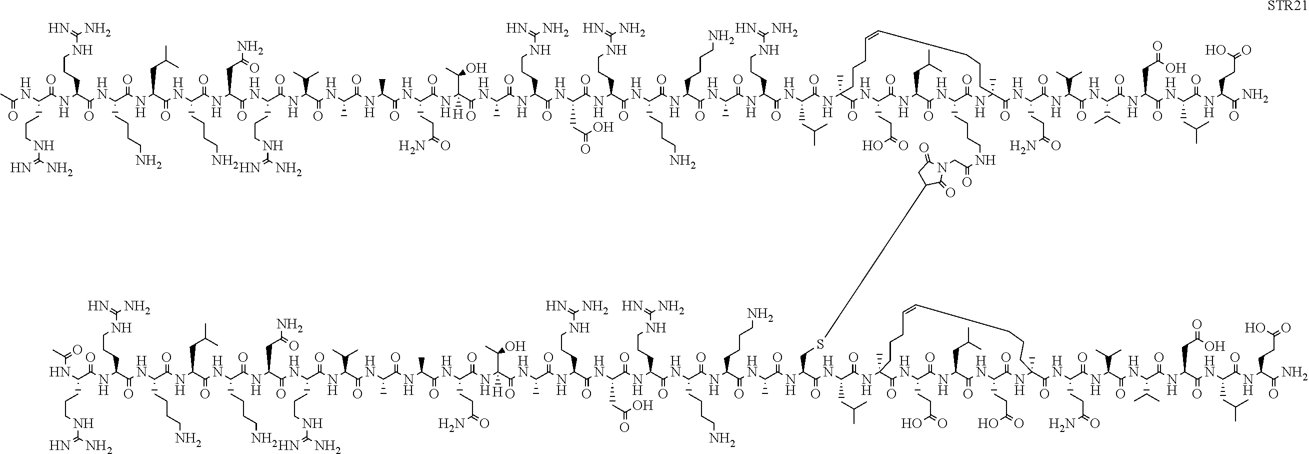

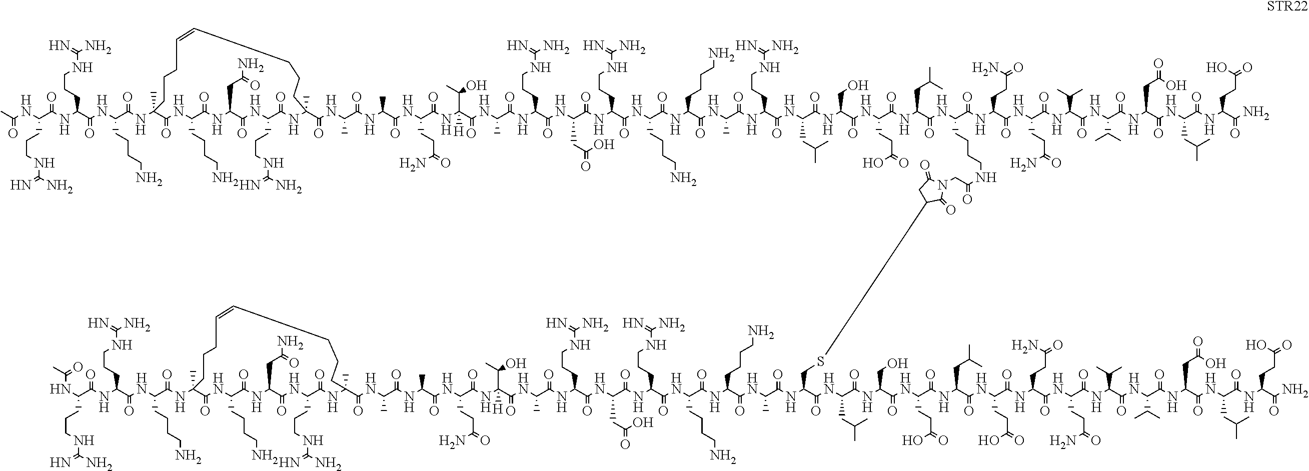

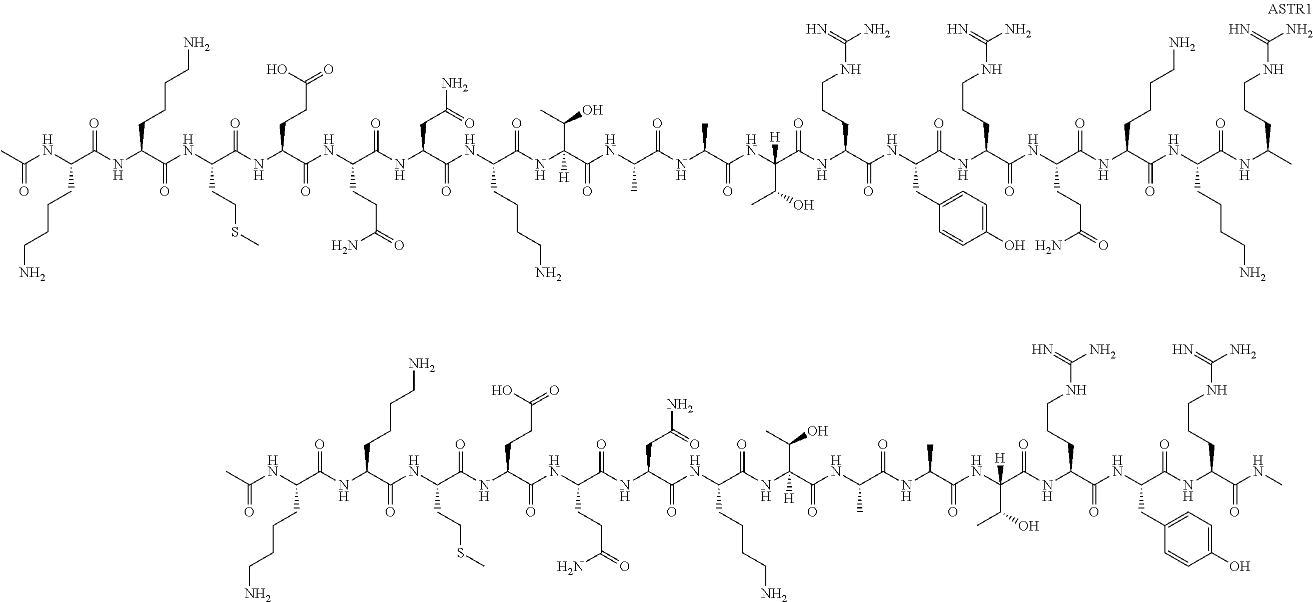

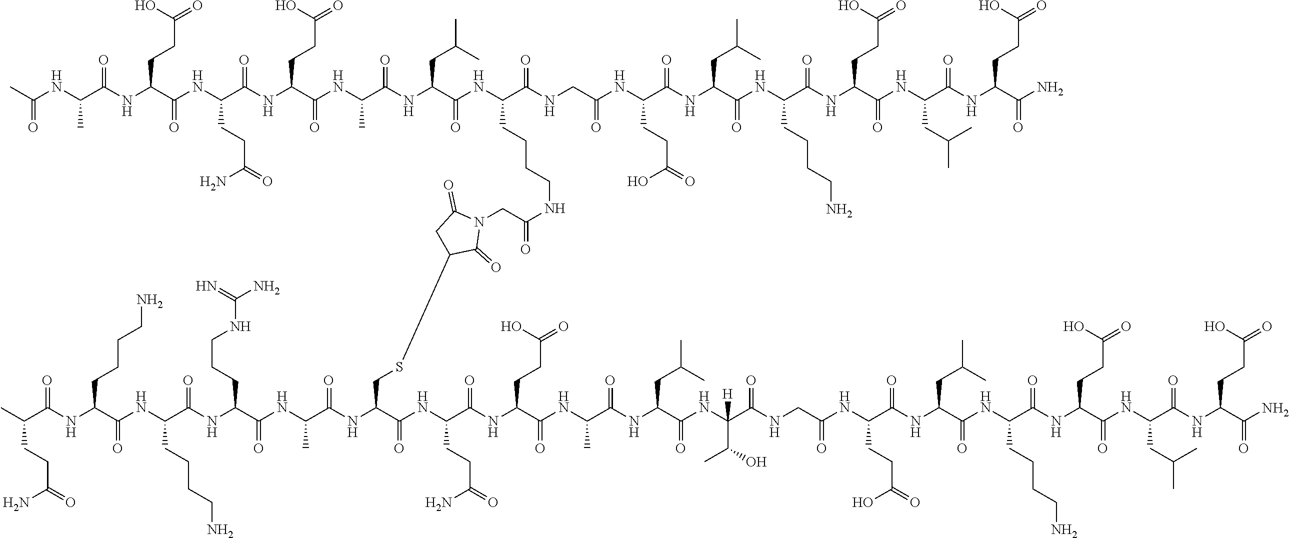

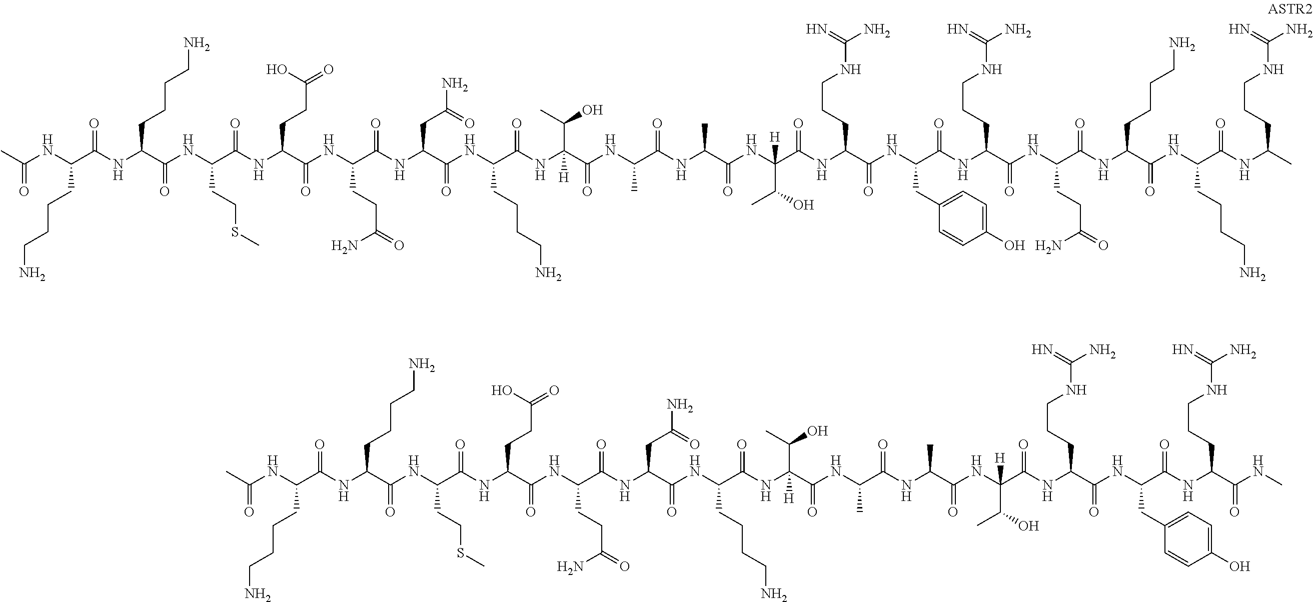

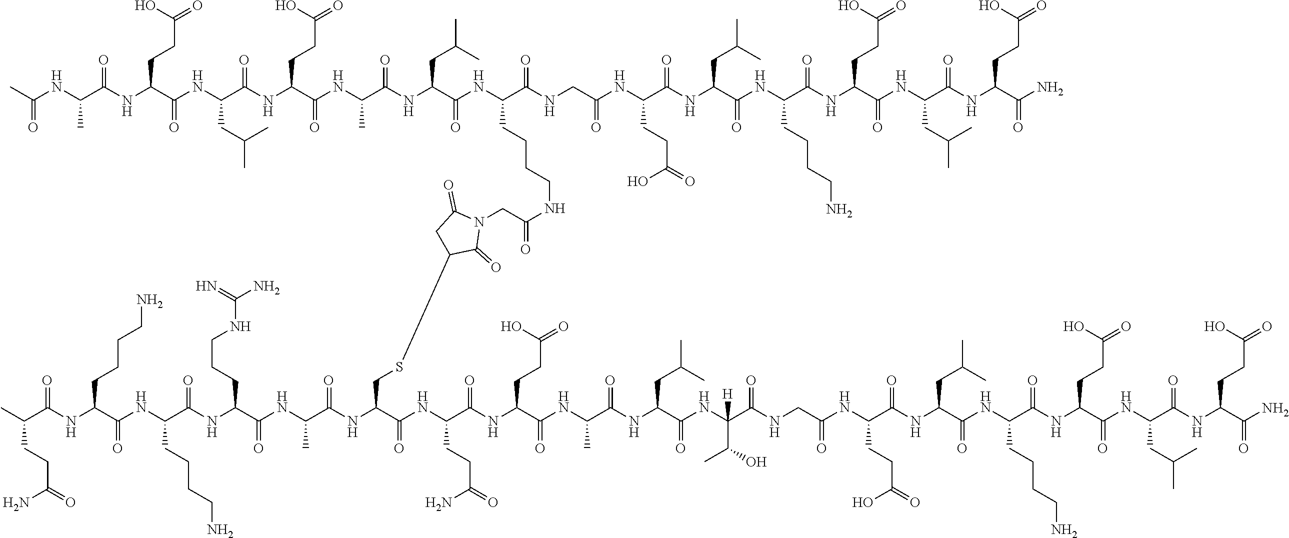

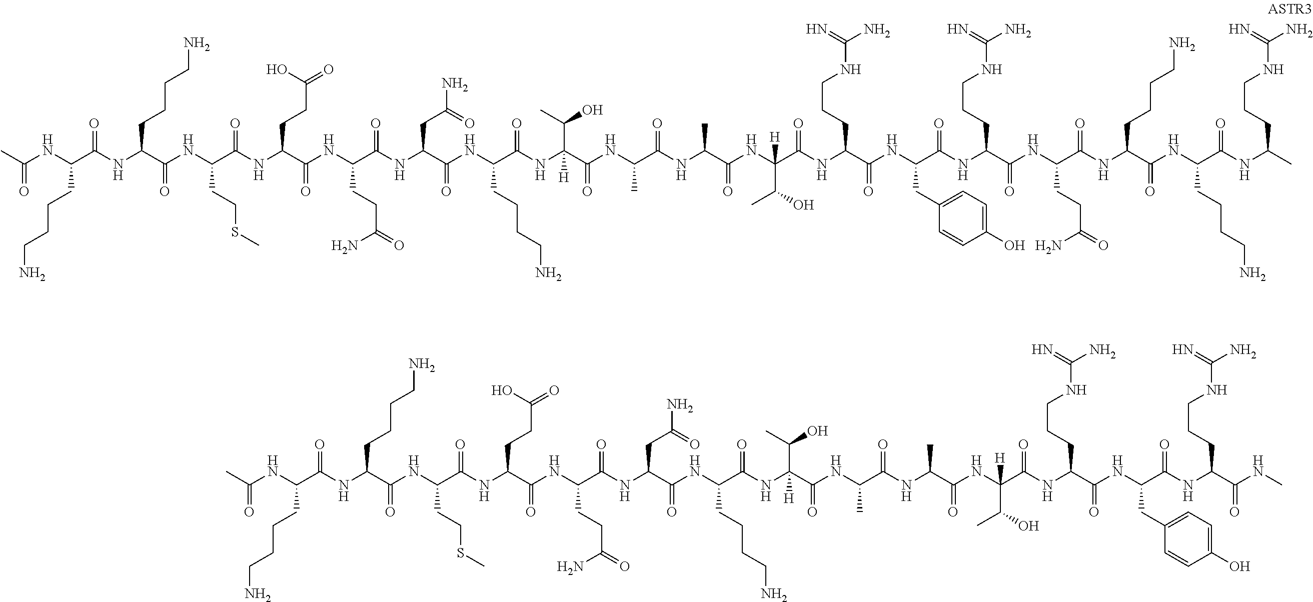

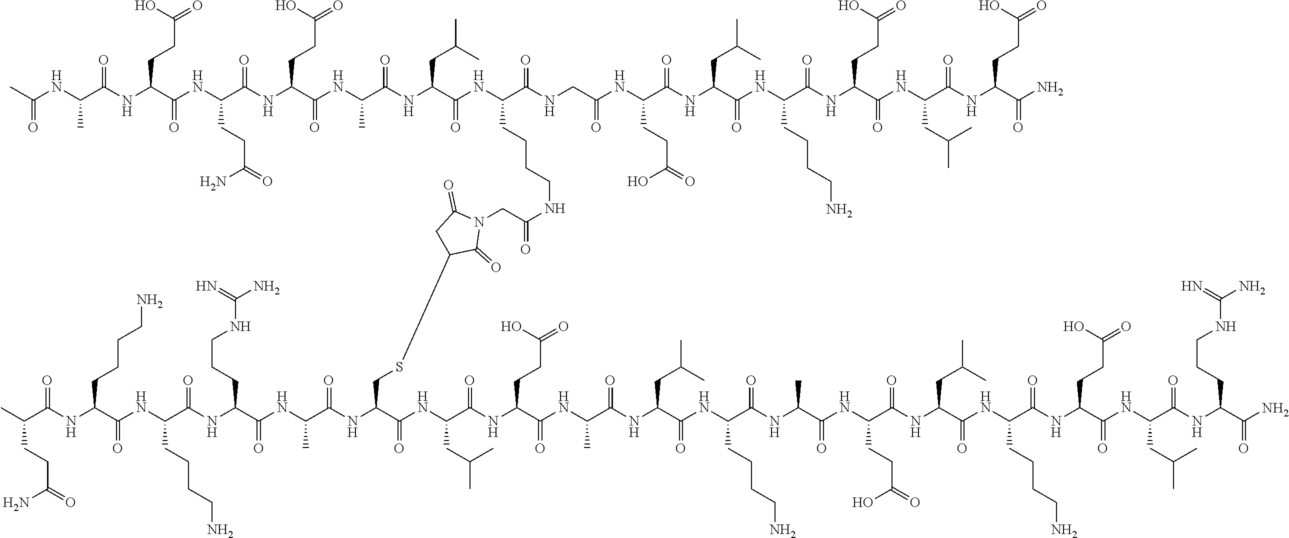

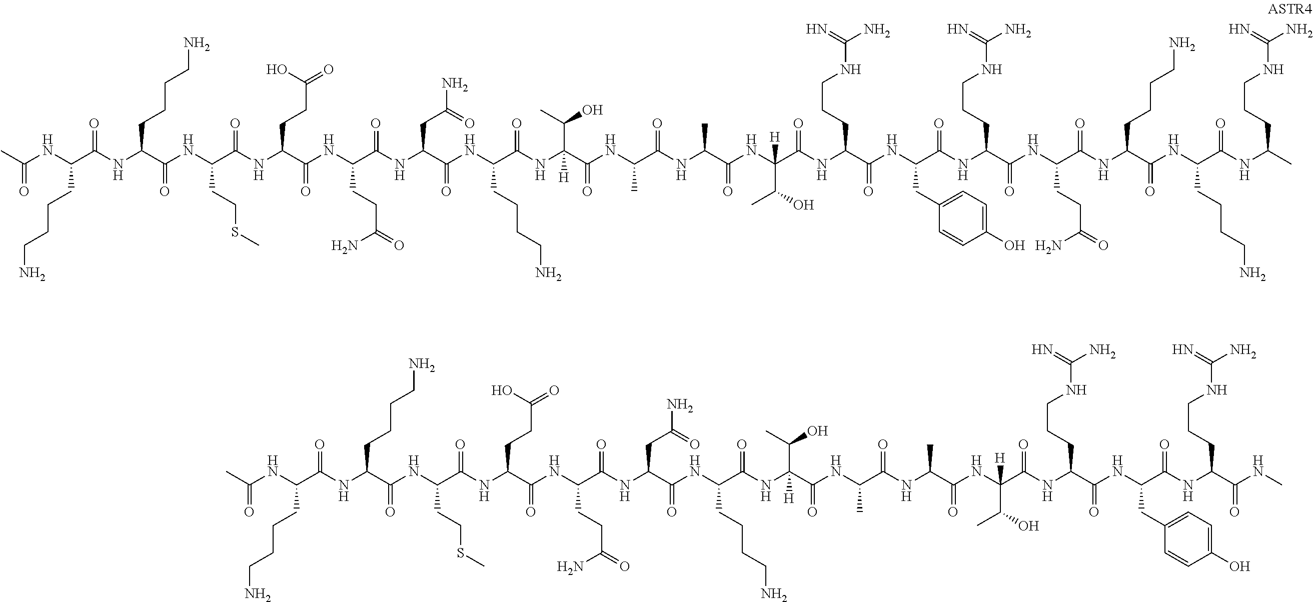

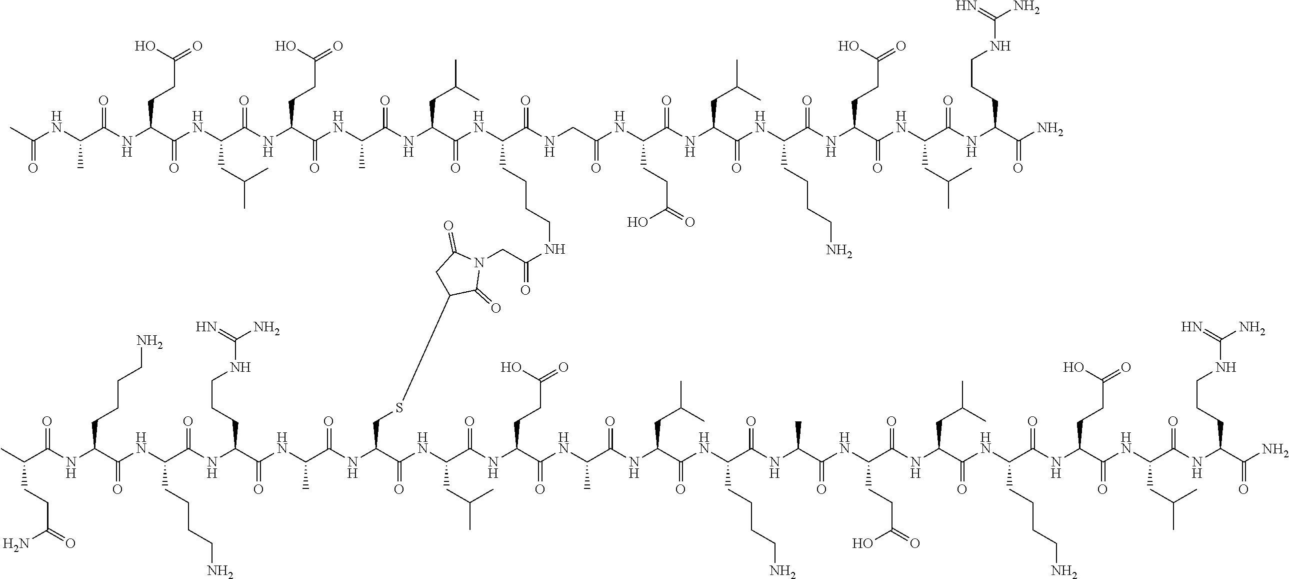

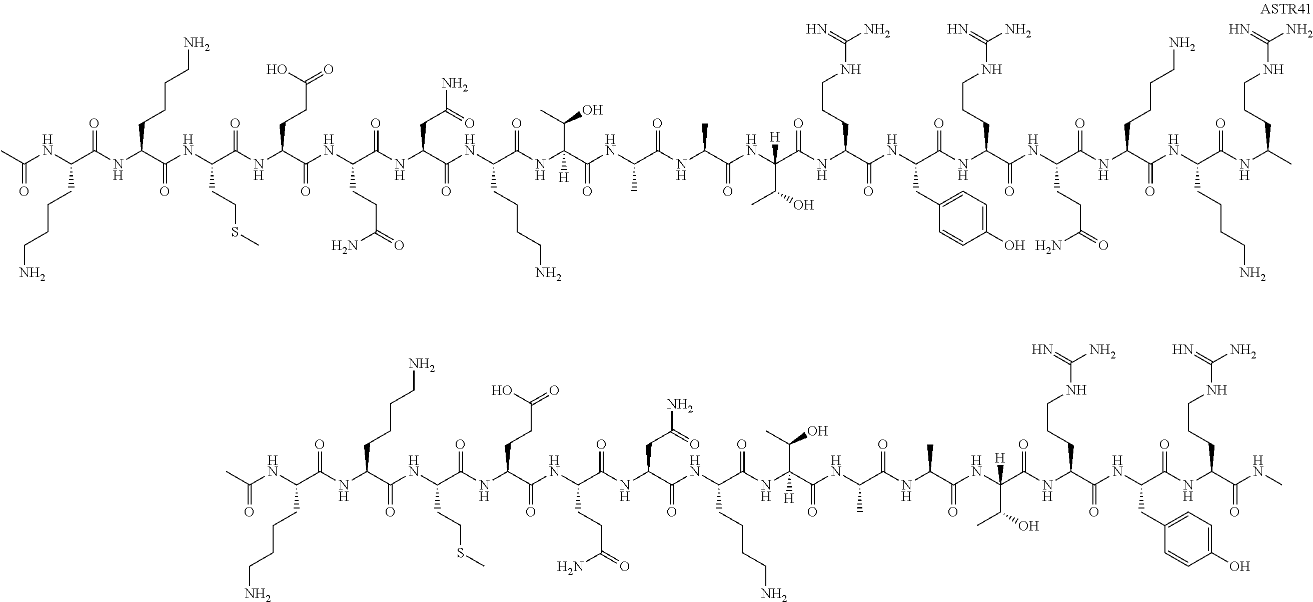

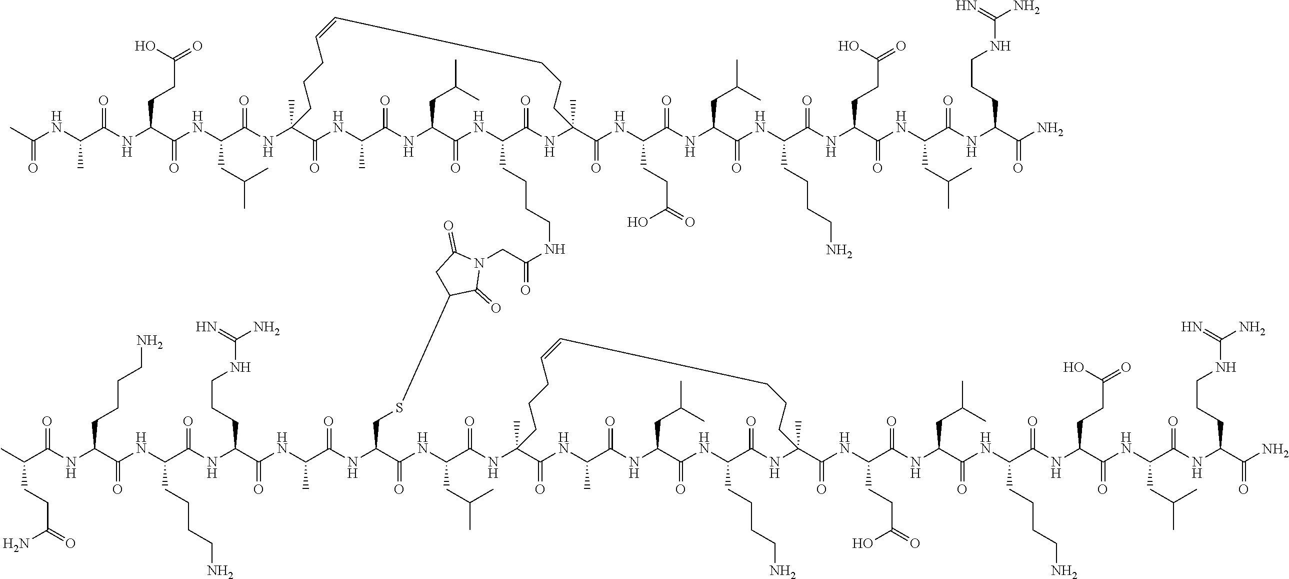

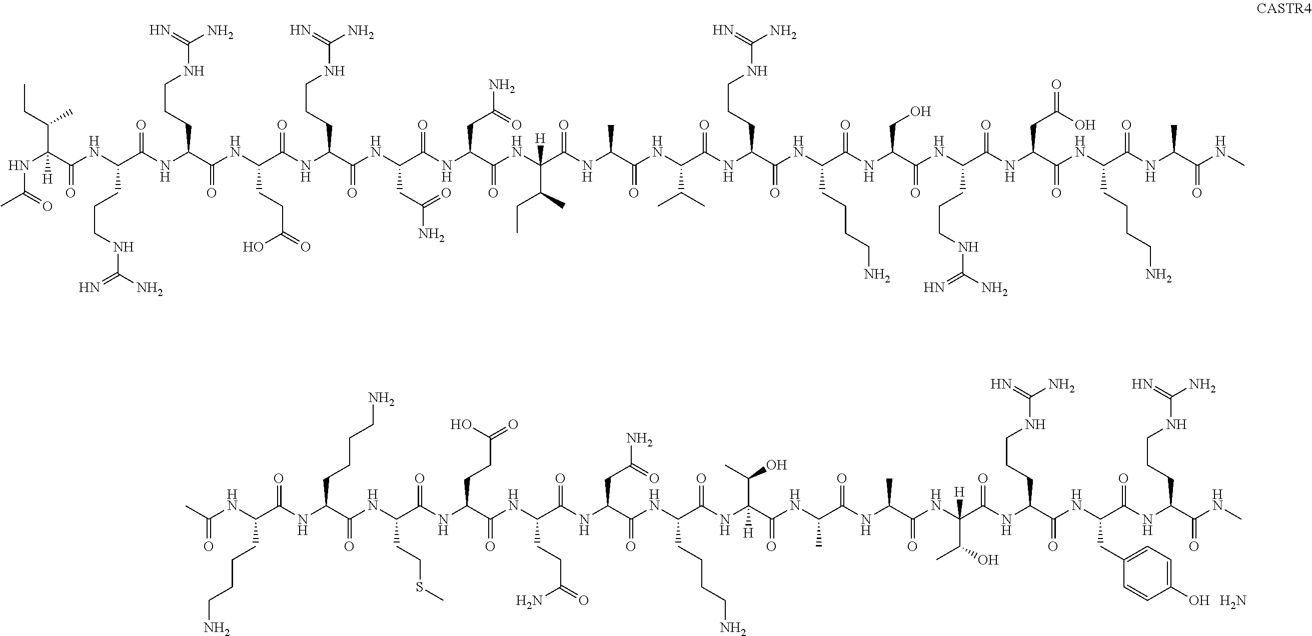

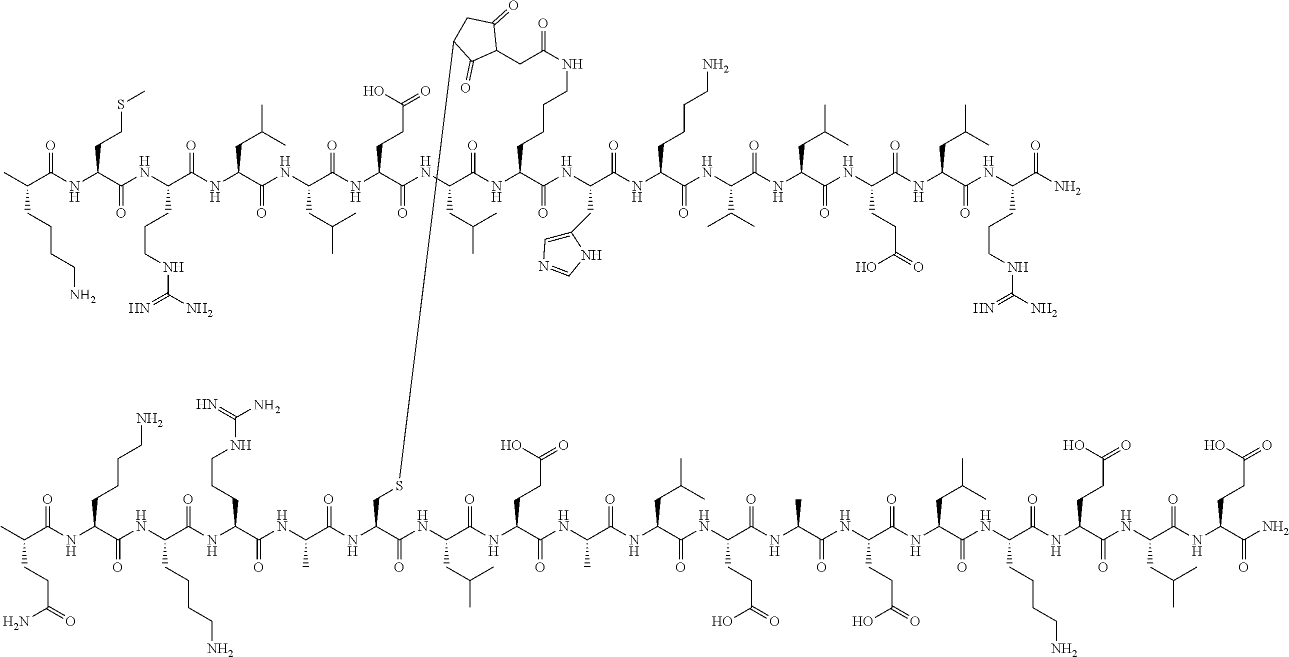

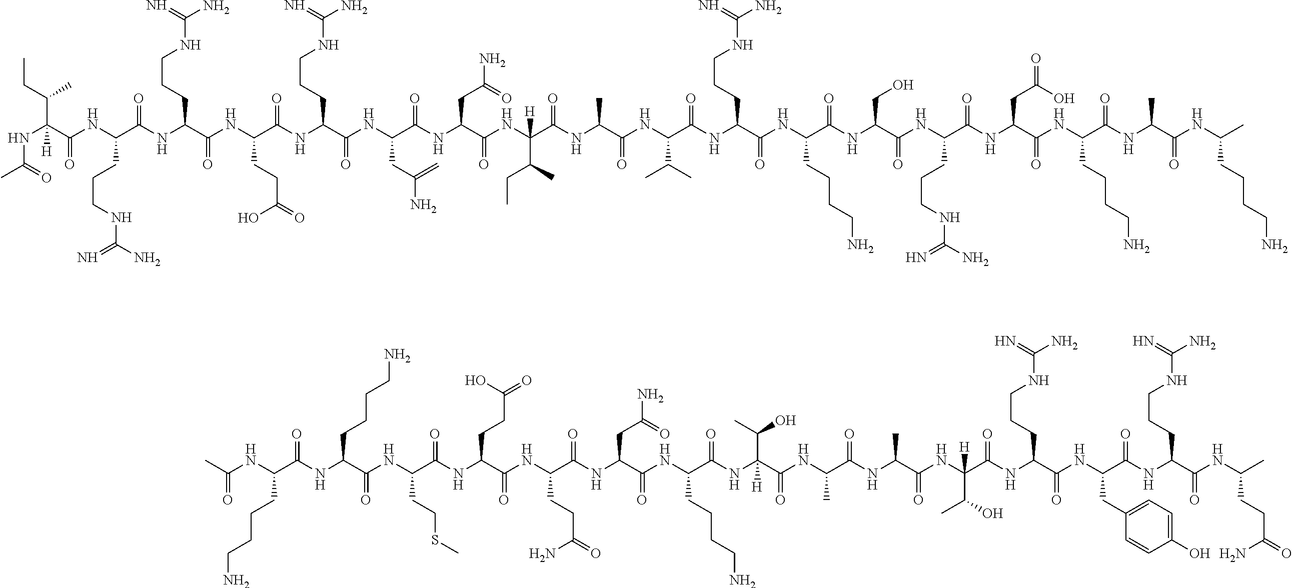

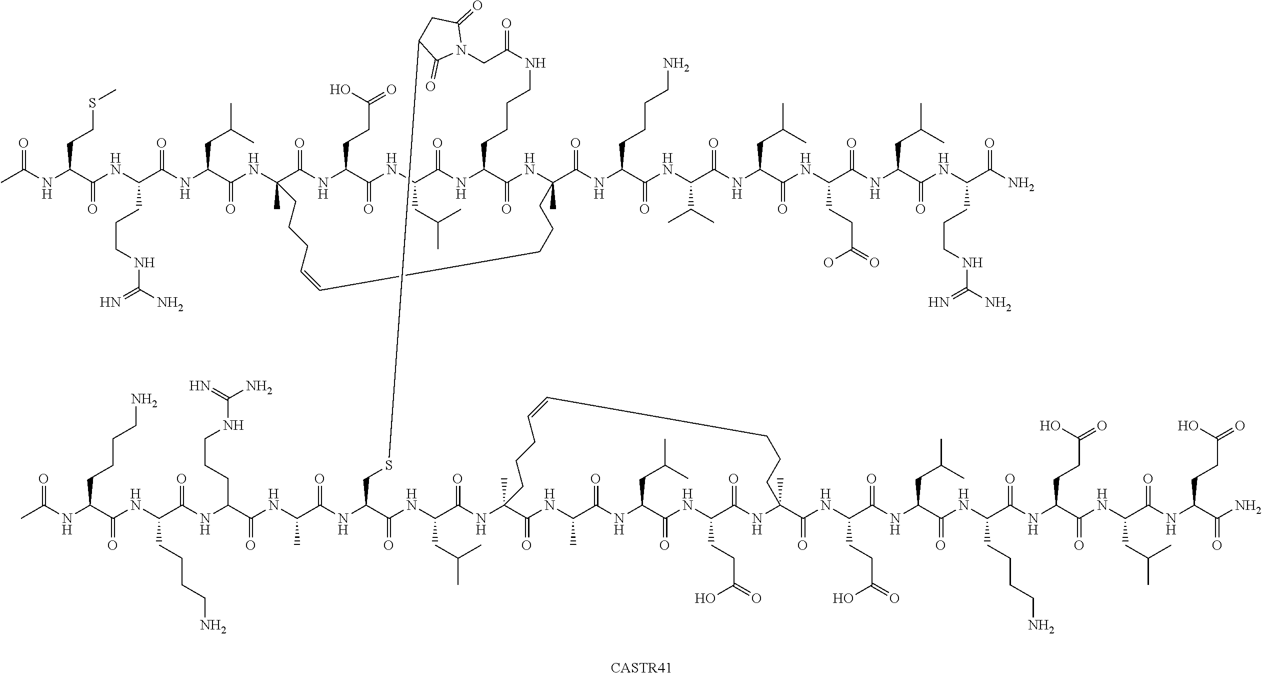

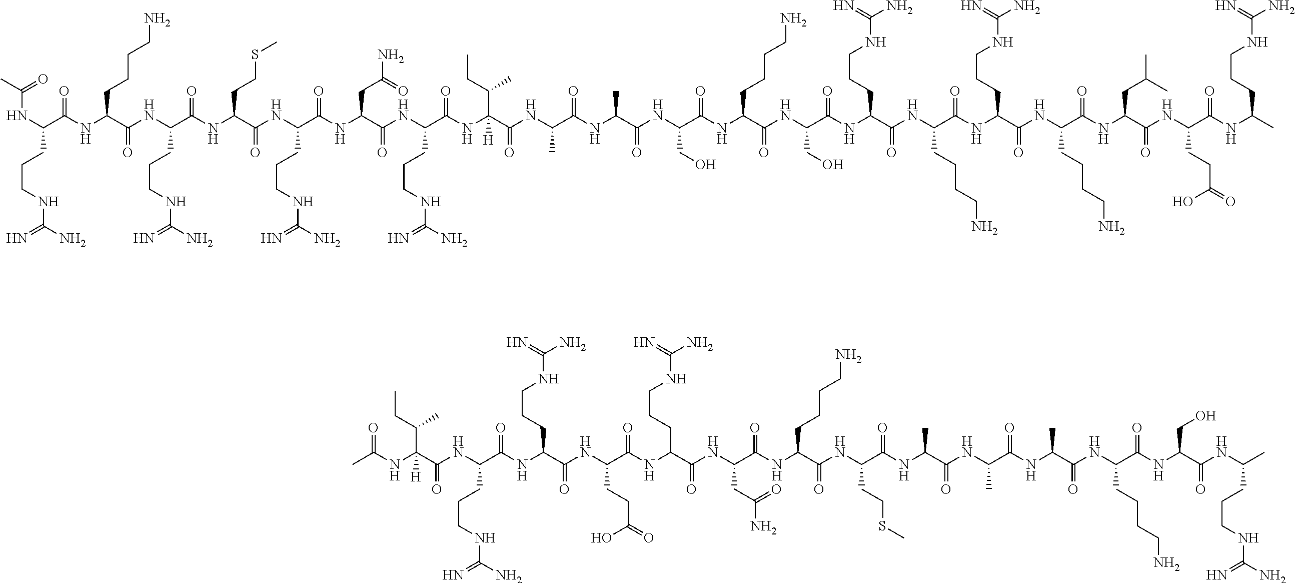

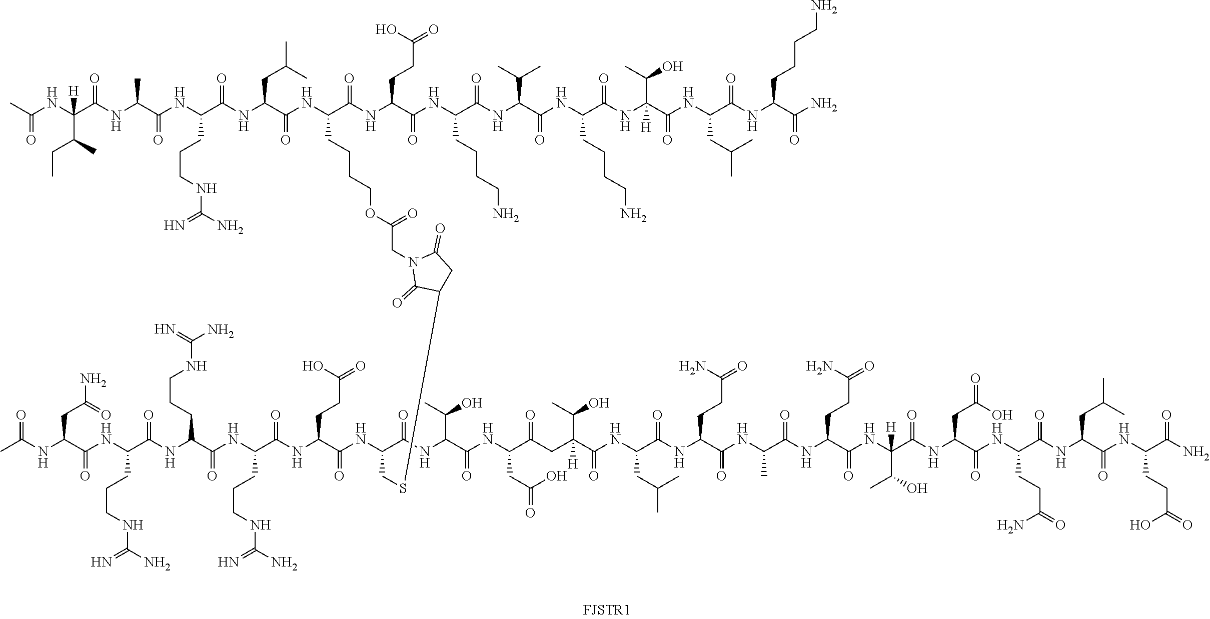

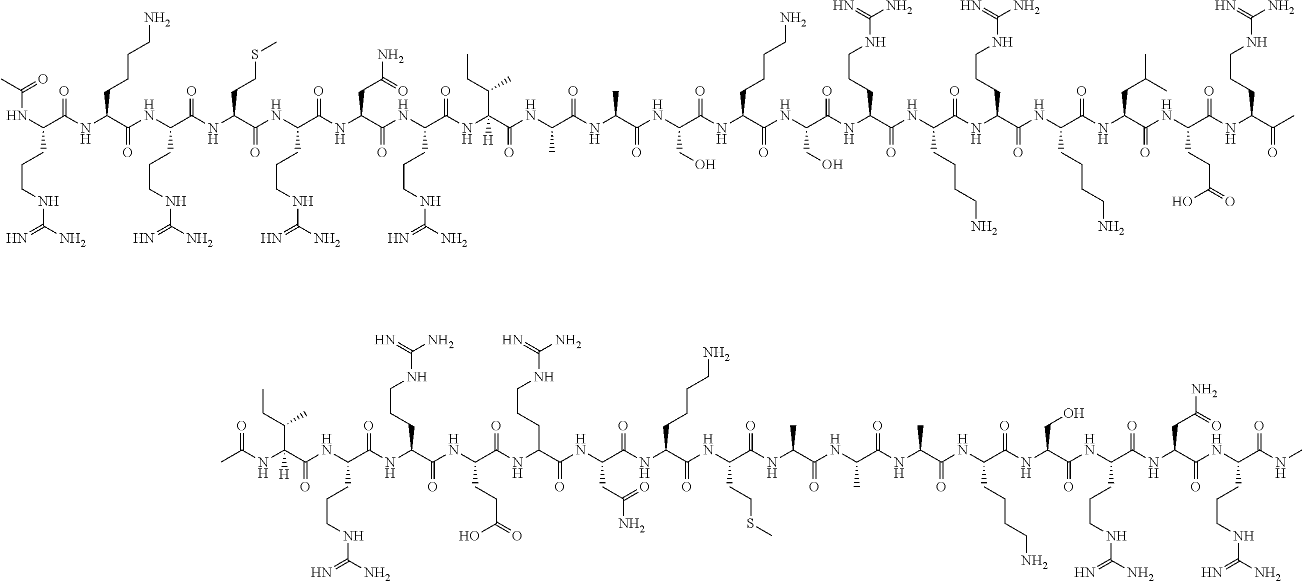

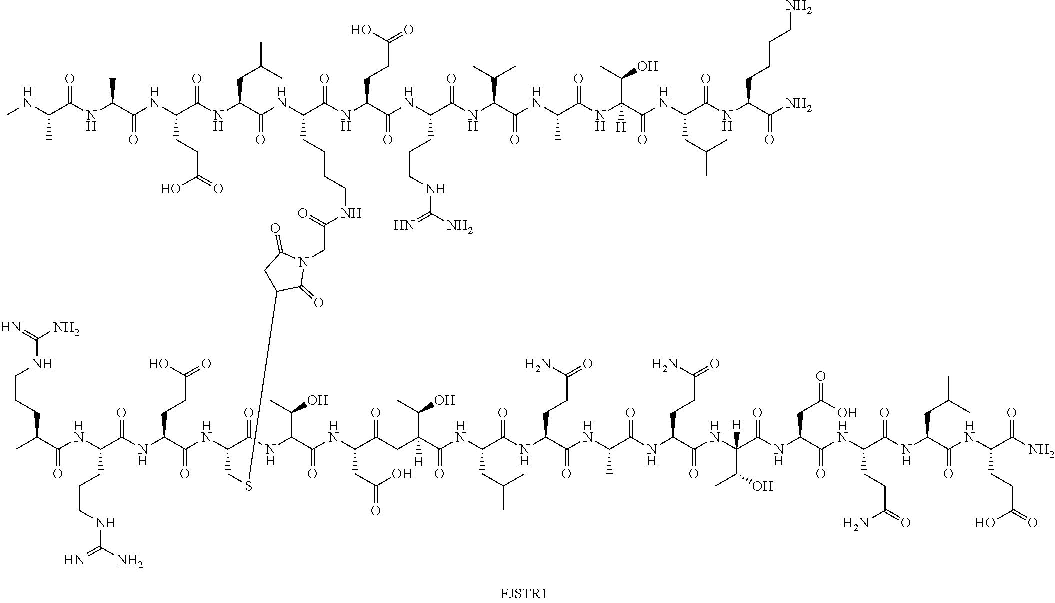

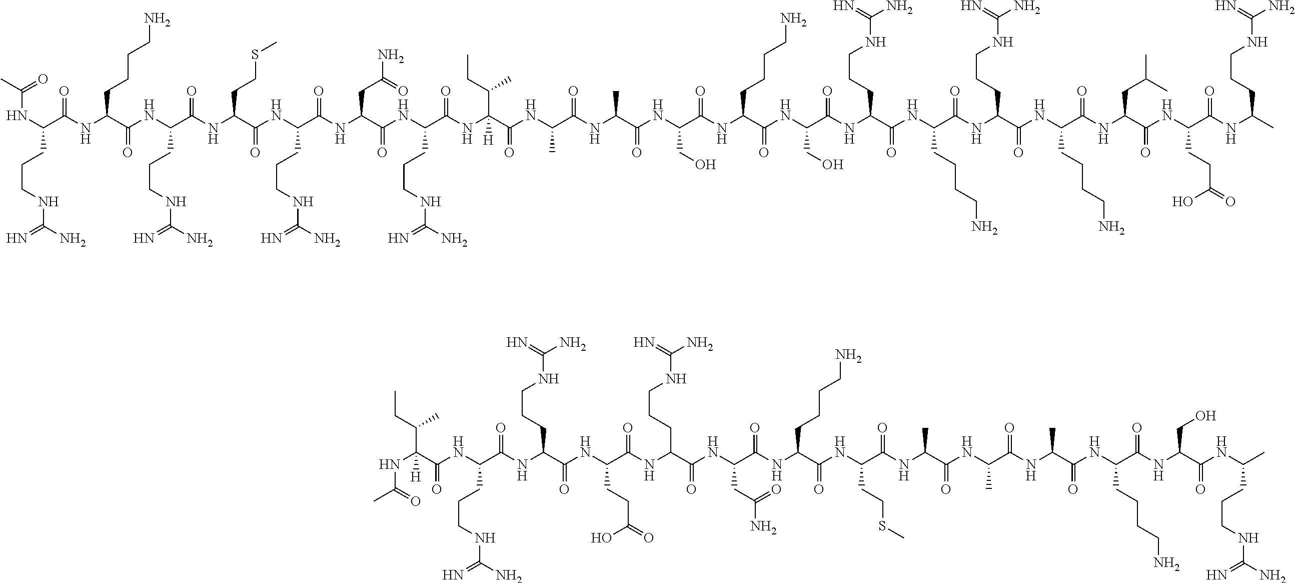

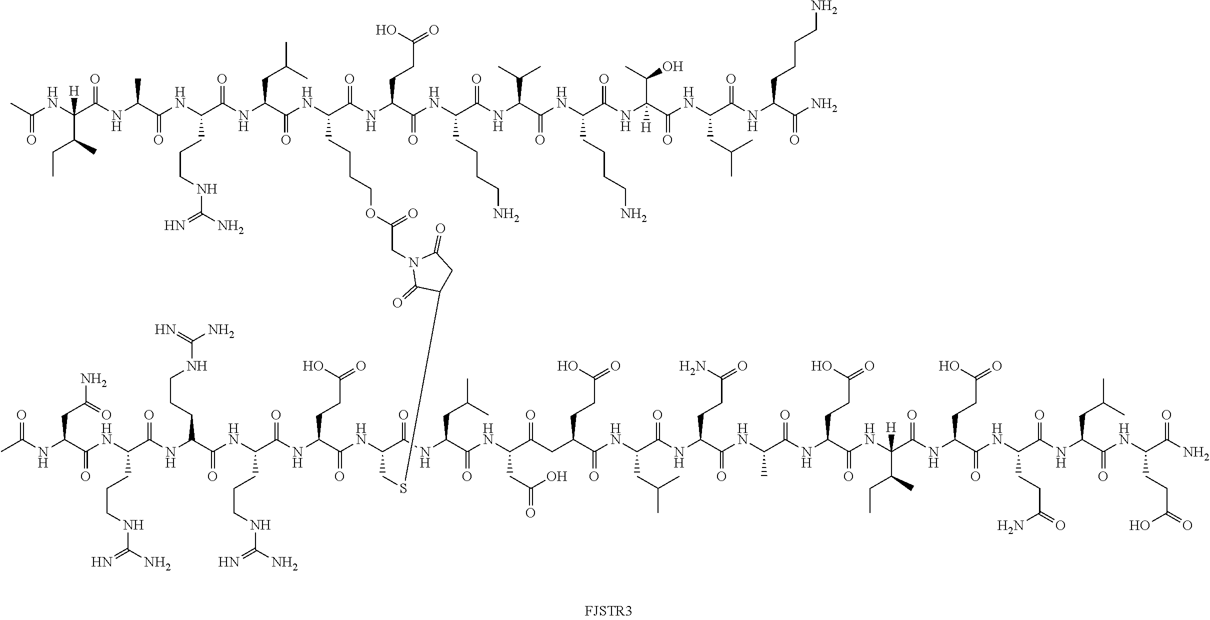

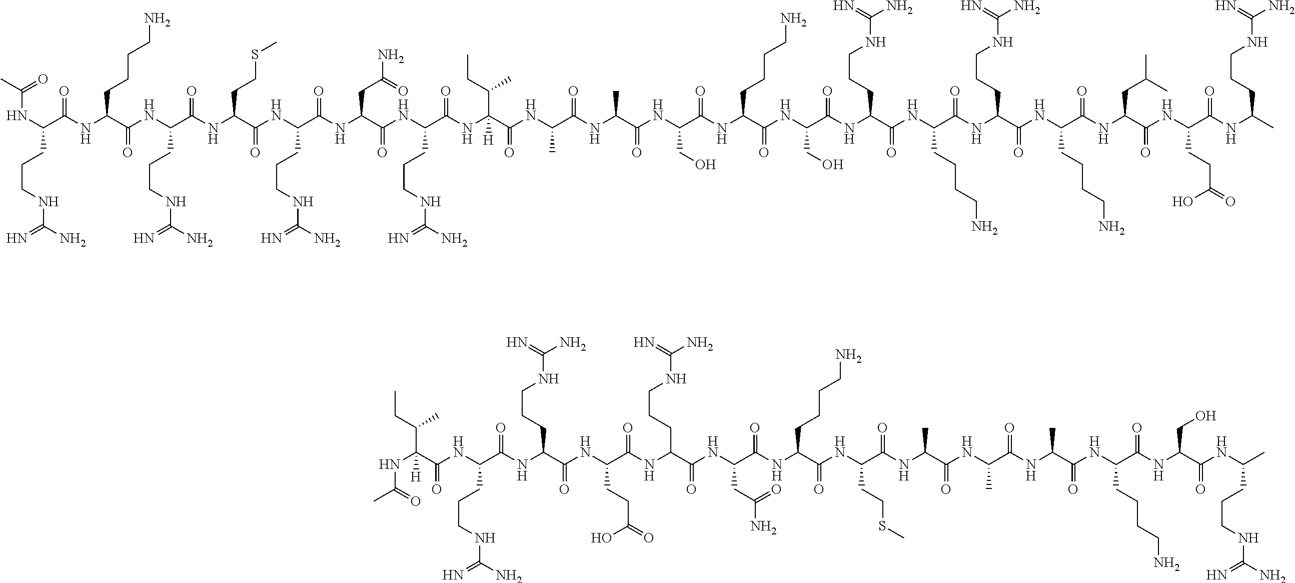

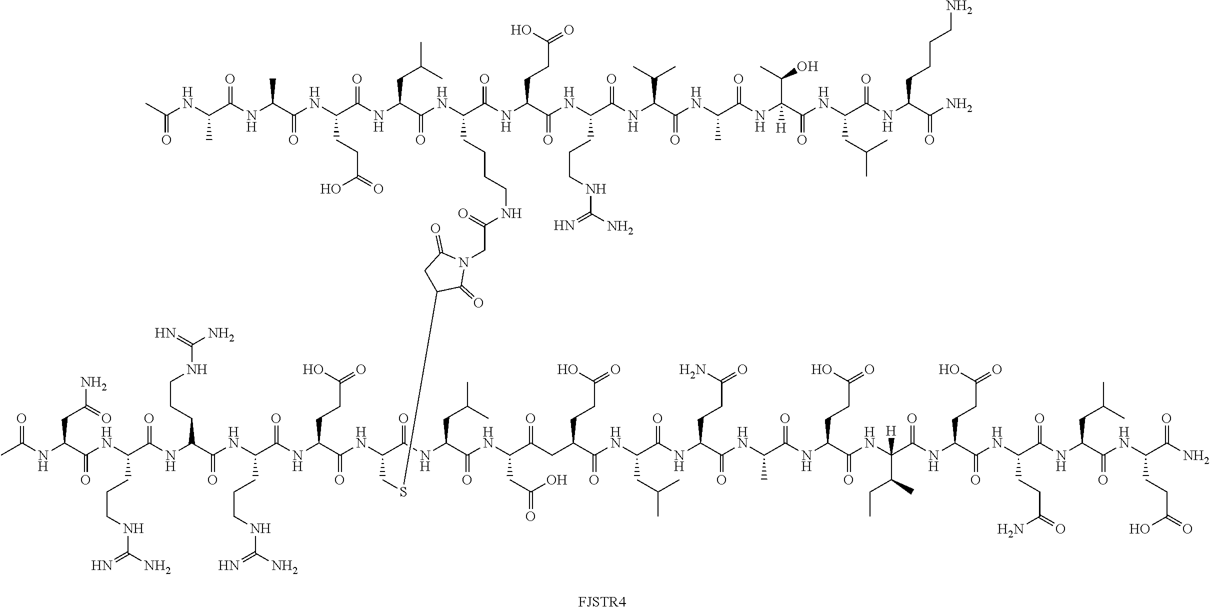

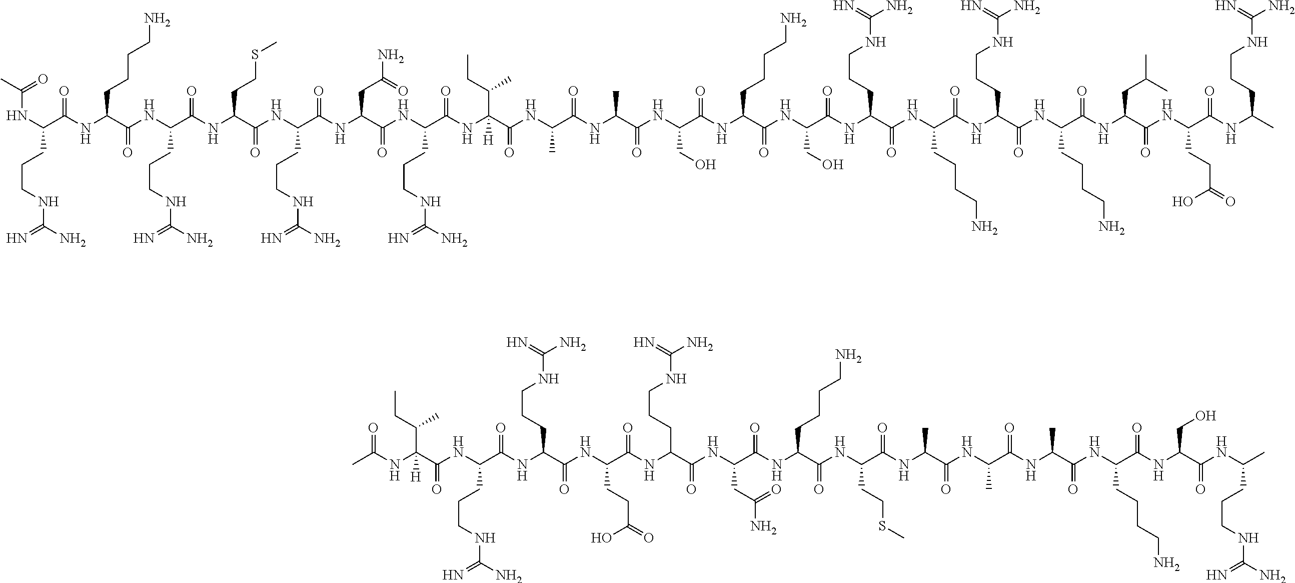

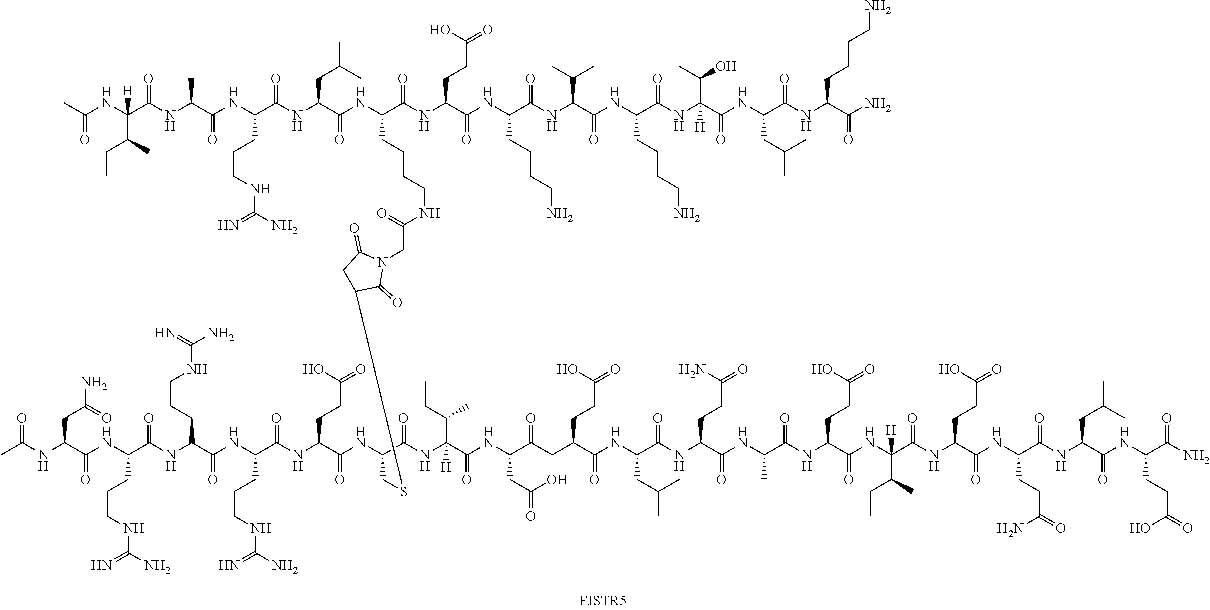

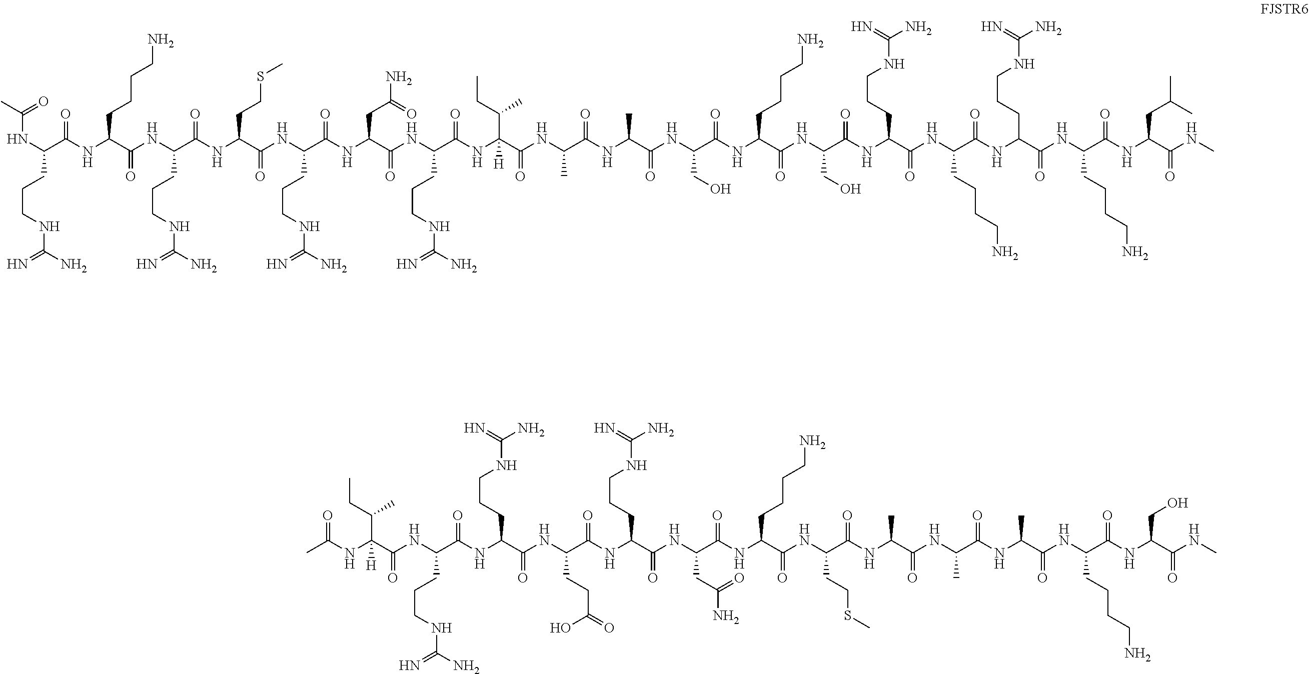

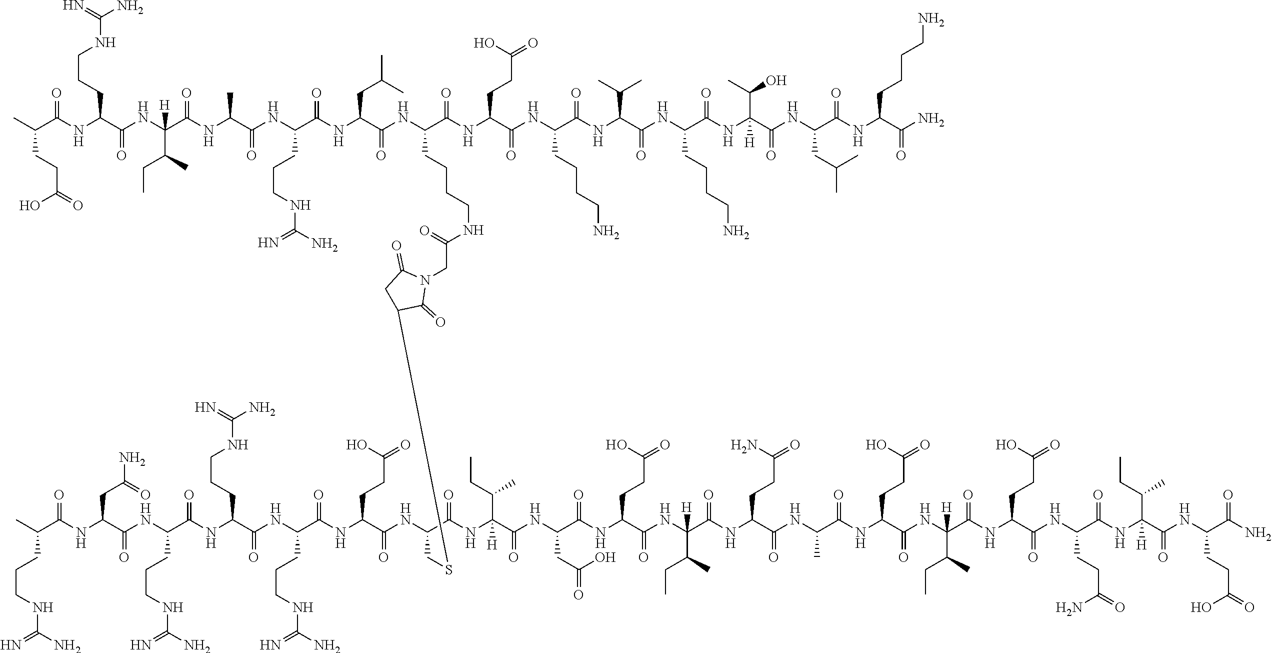

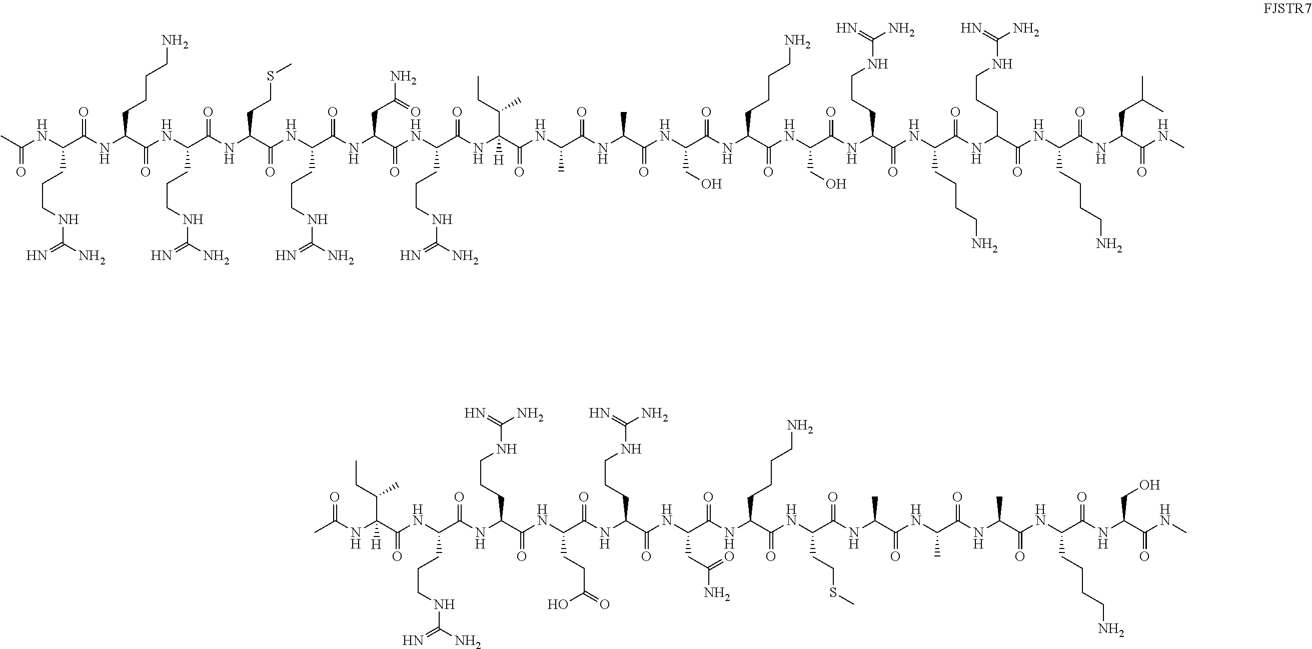

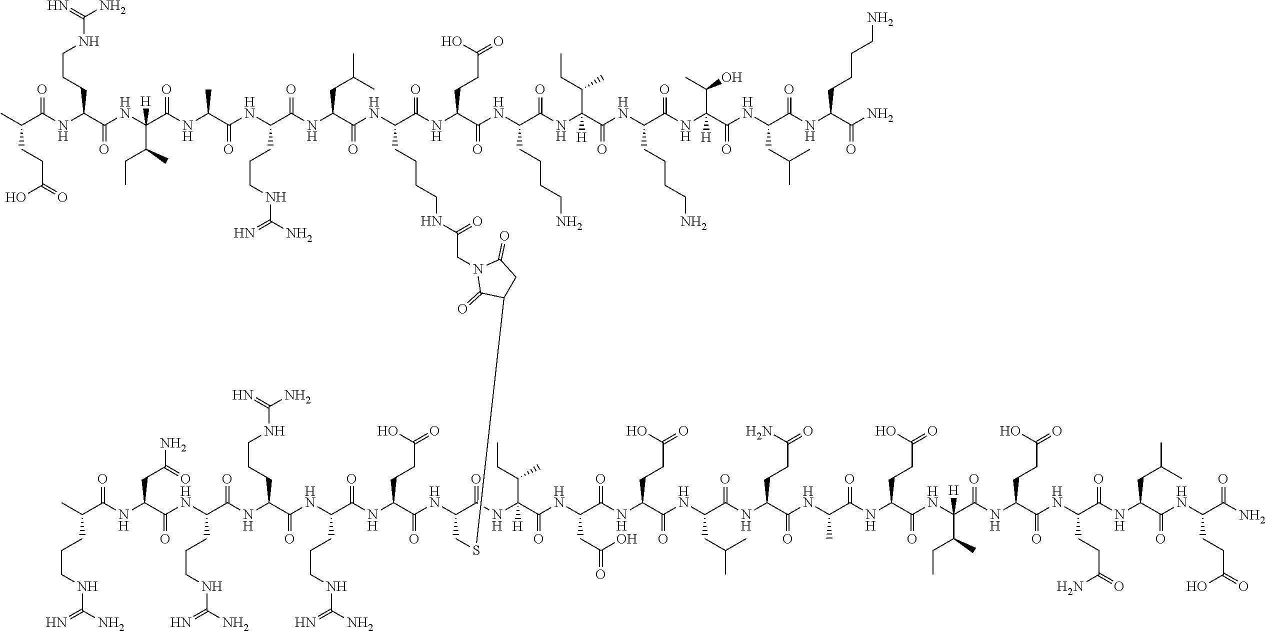

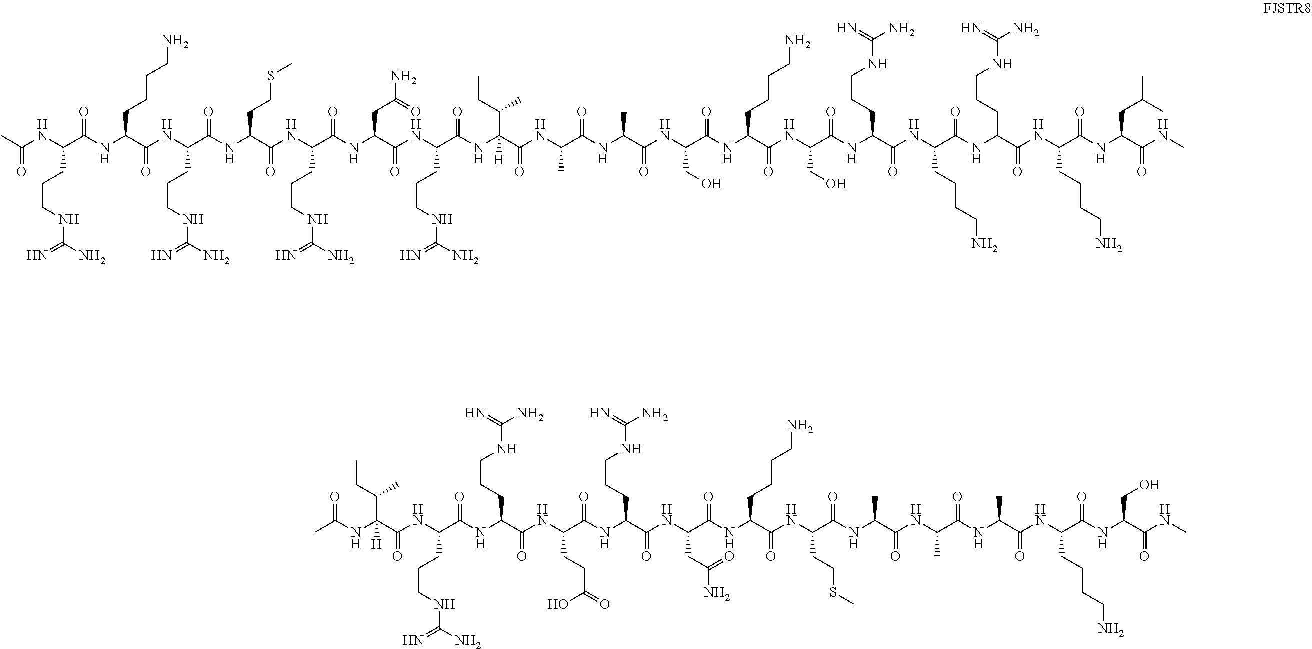

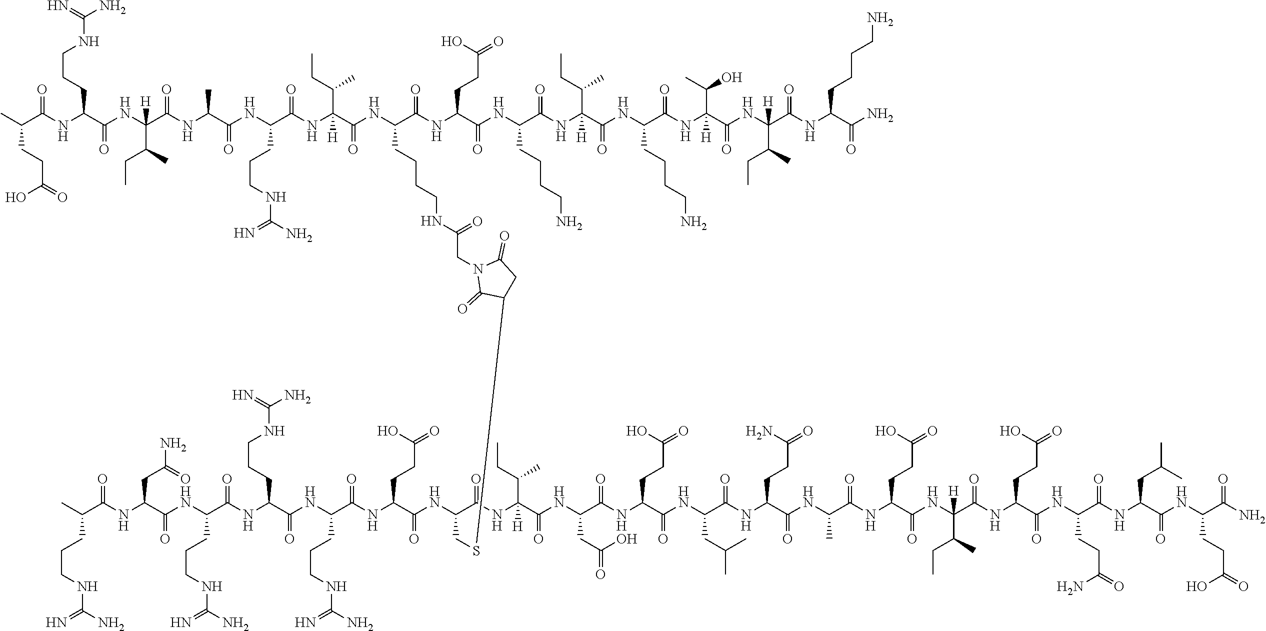

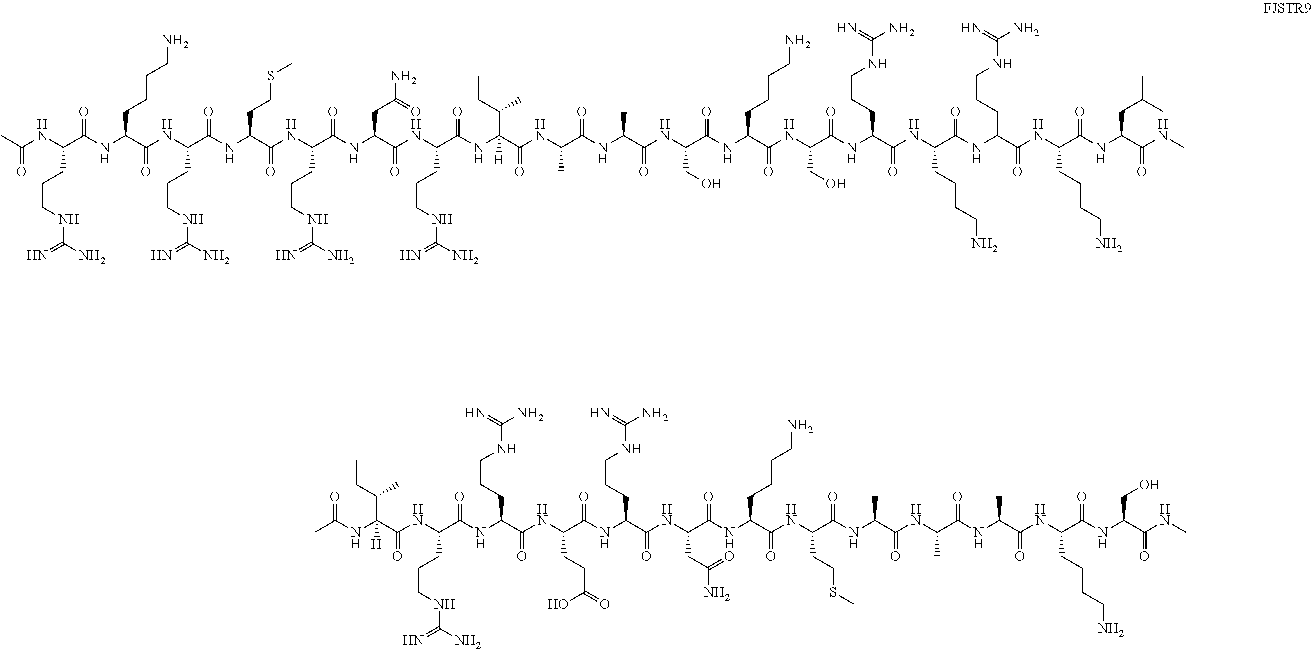

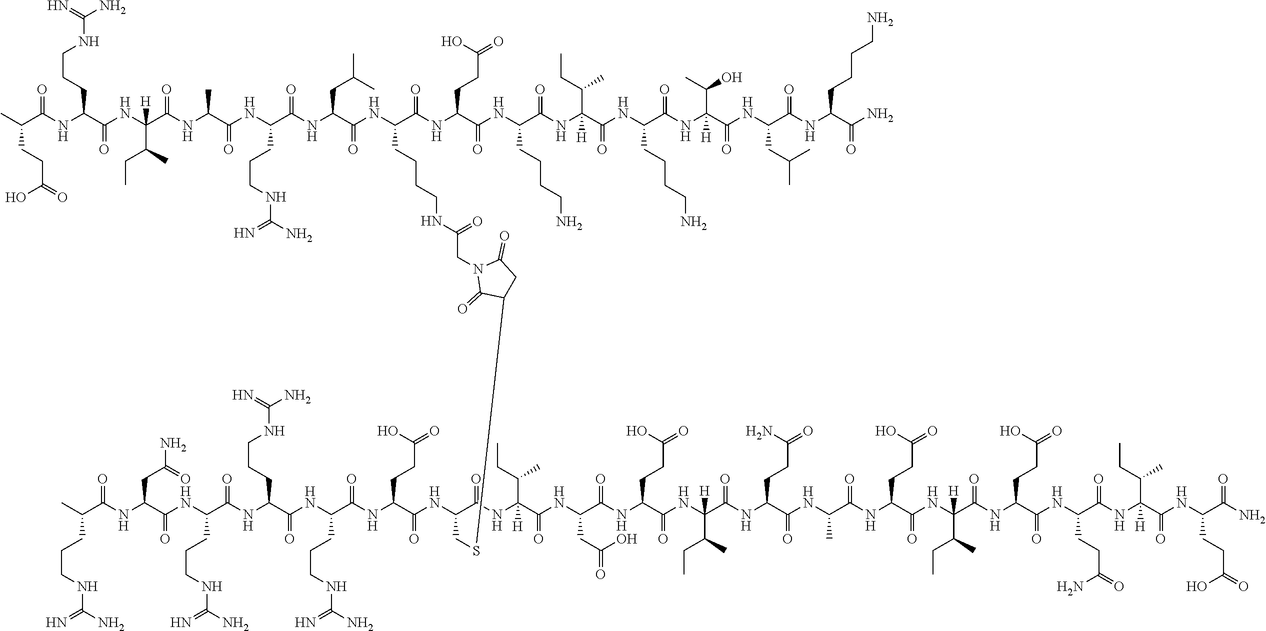

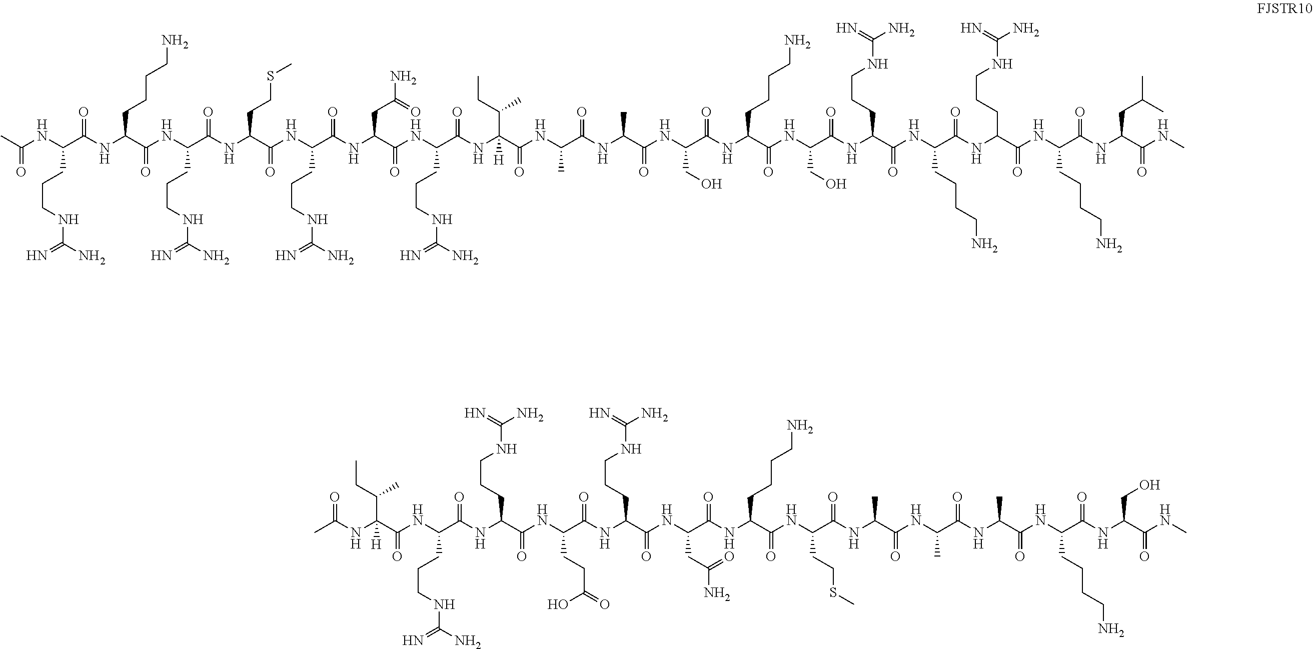

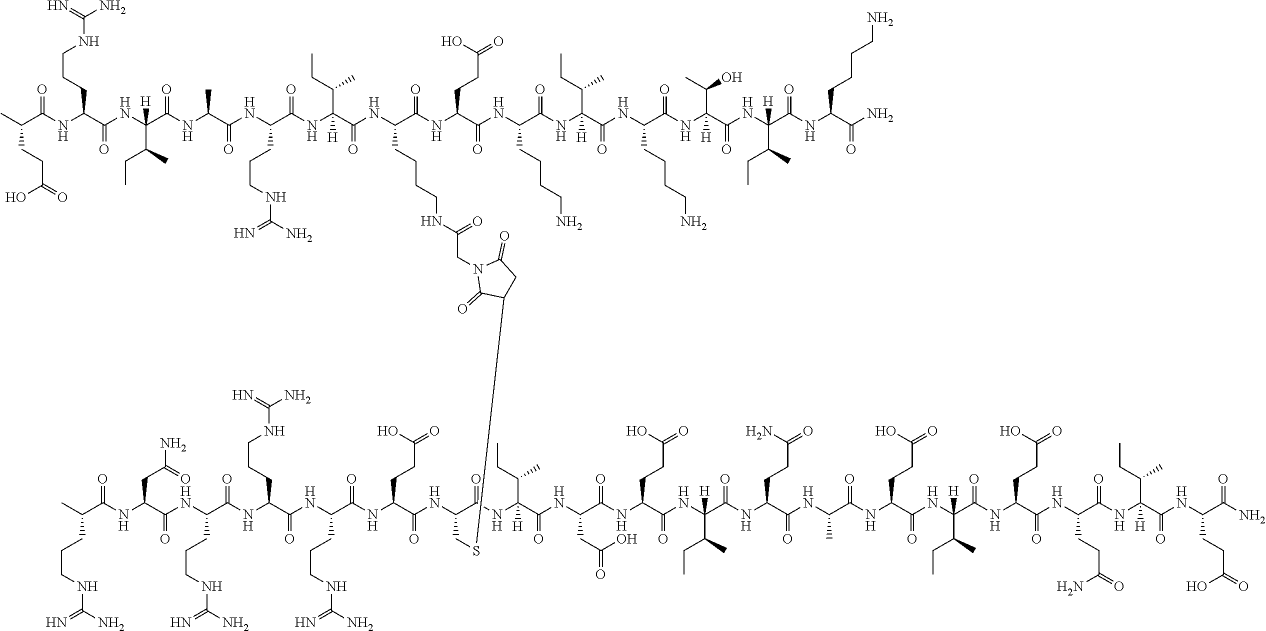

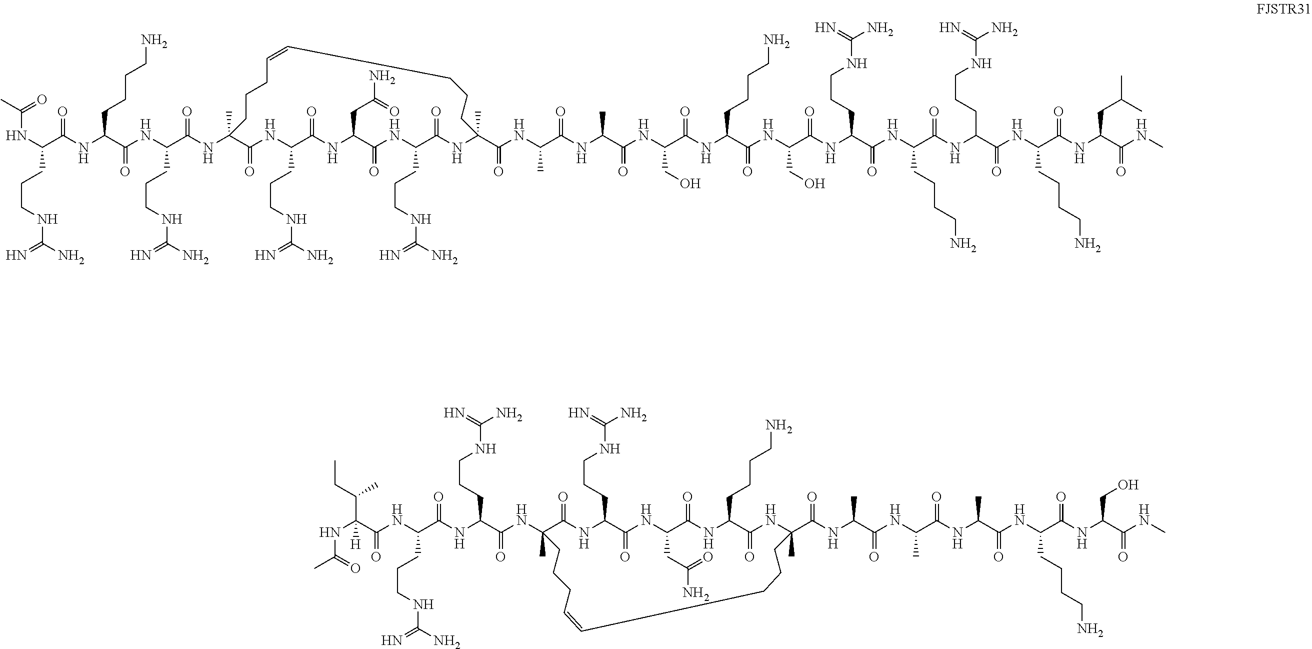

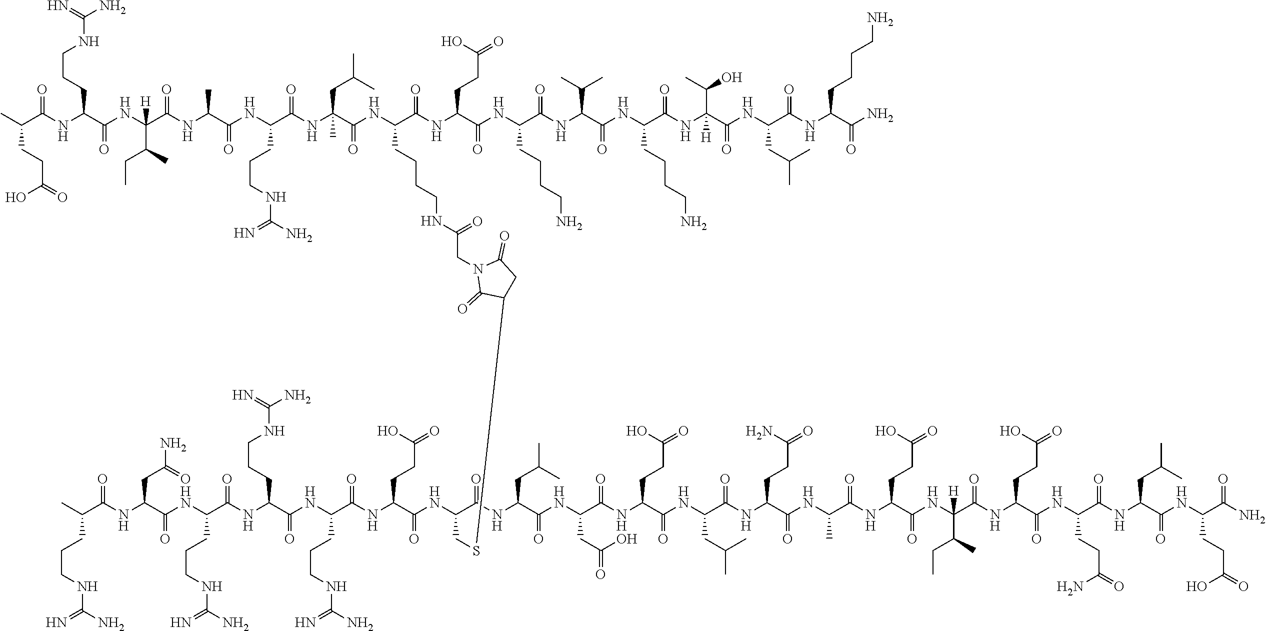

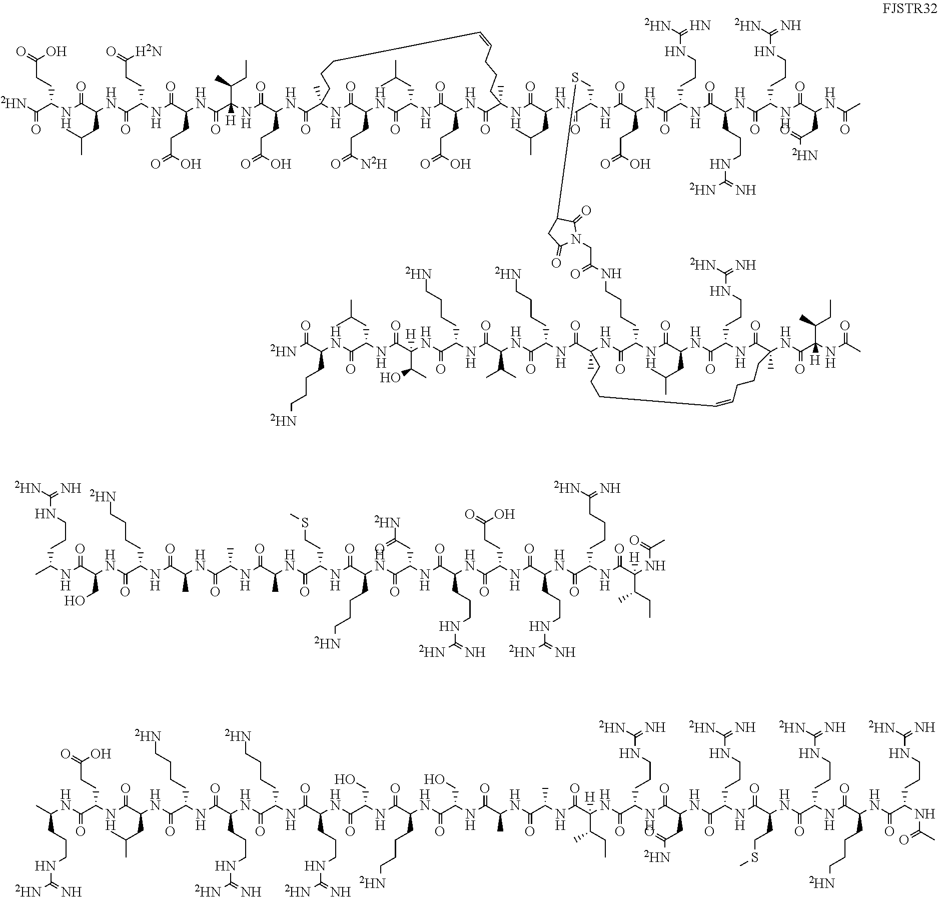

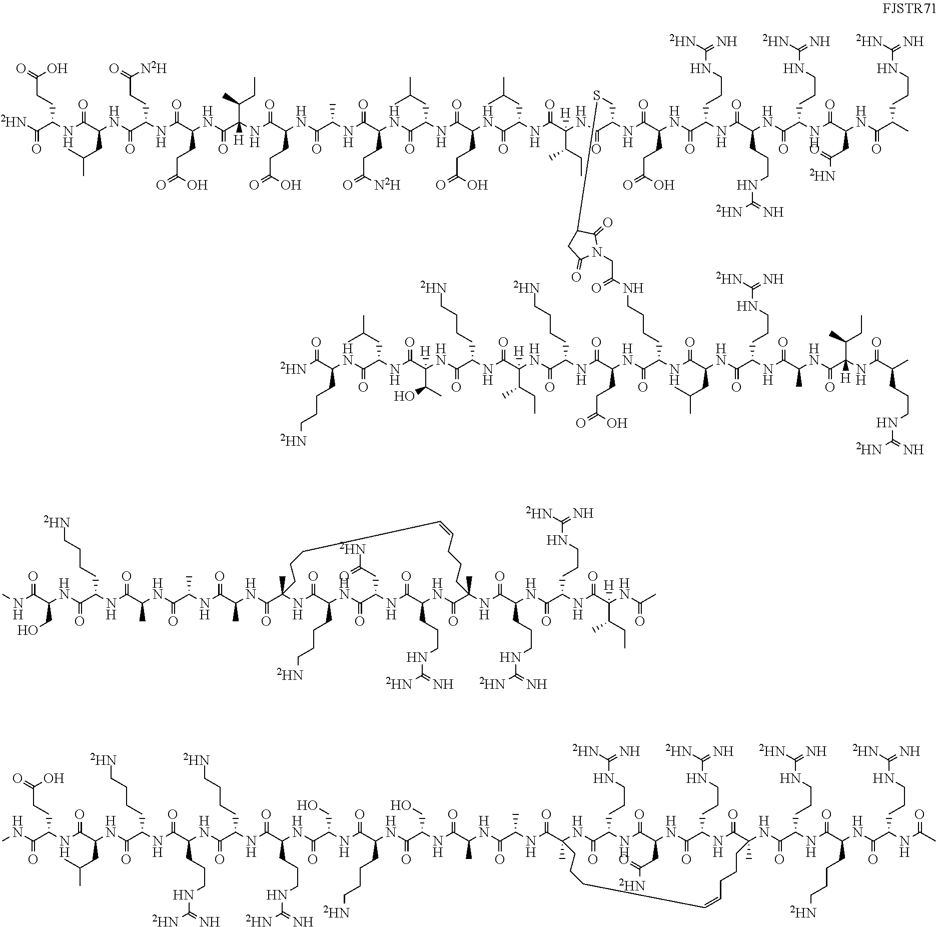

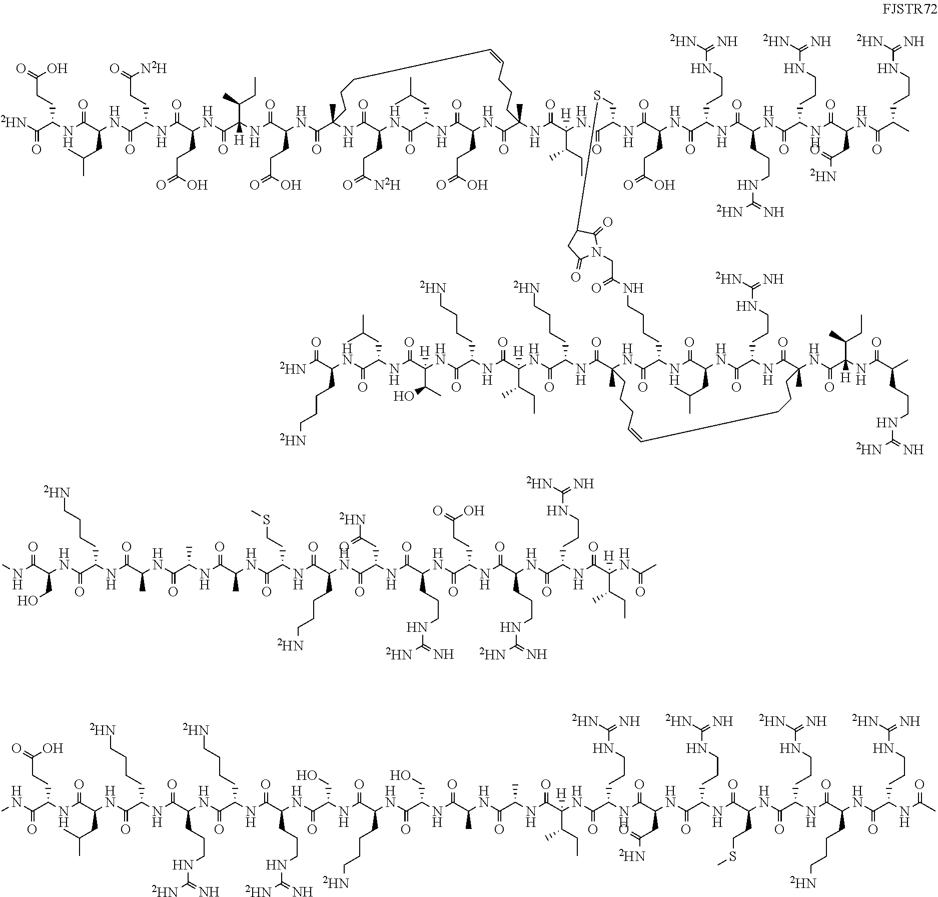

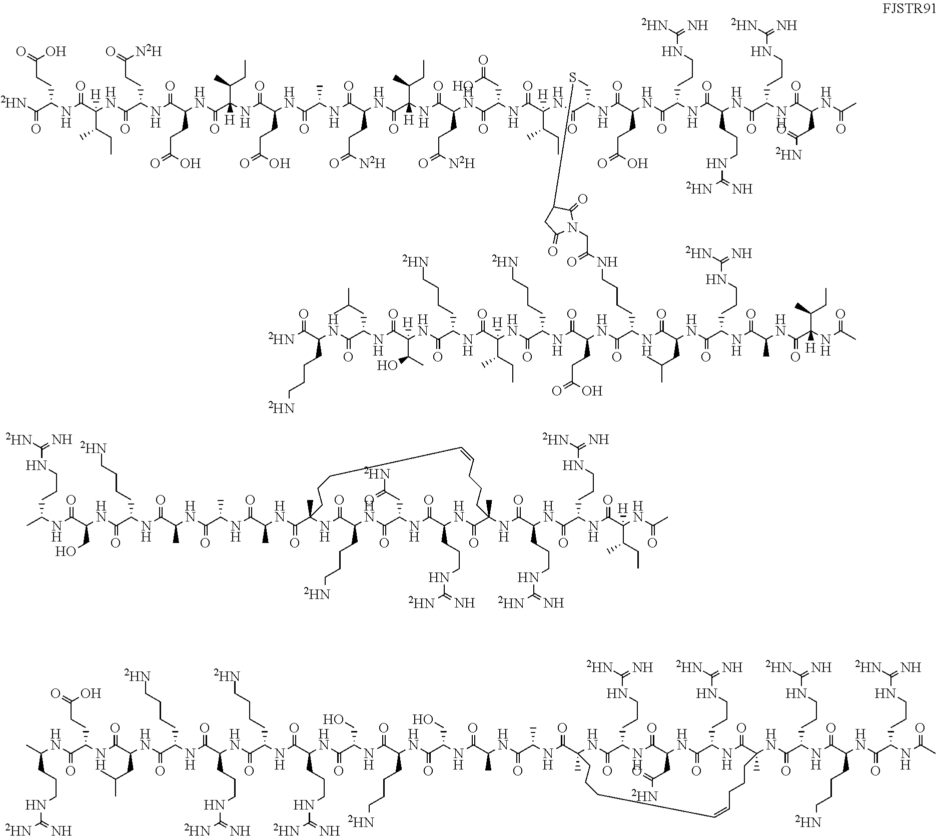

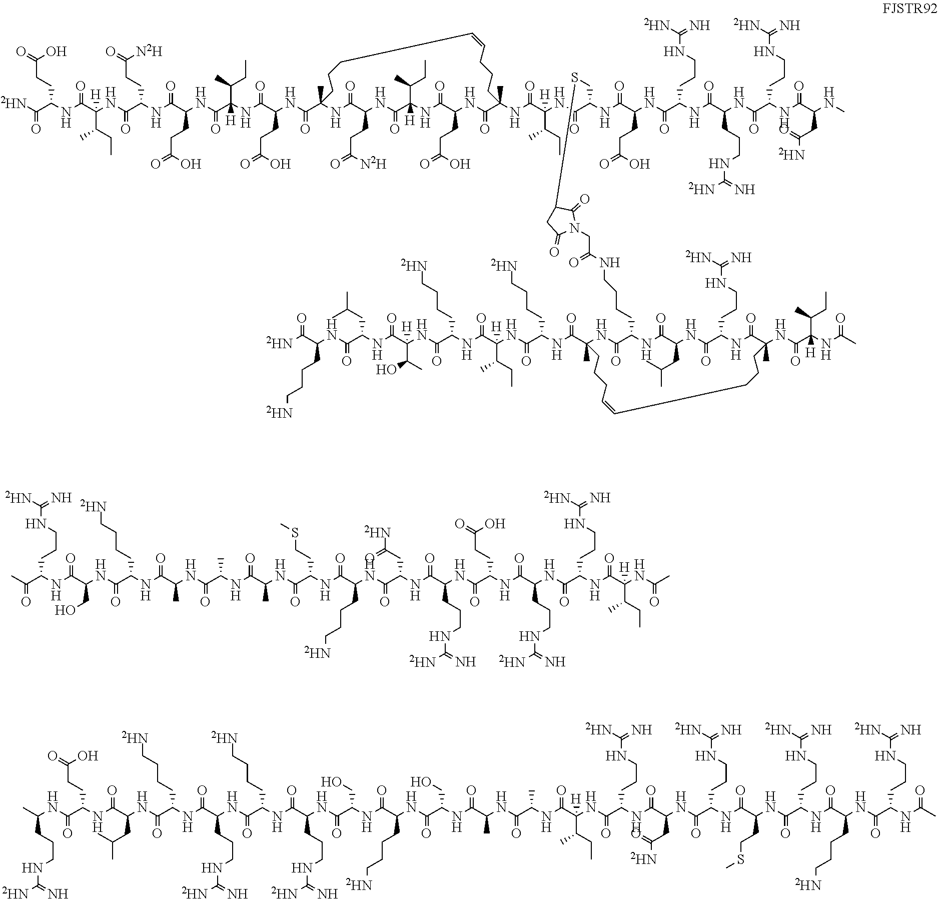

In some aspects, ASTRs (including ASTR1, ASTR3, ASTR2, ASTR4, and/or ASTR41) comprise a sequence from ATF4 that spans a defined region of basic and leucine zipper domain, and that is connected to a second monomer from the same ATF4 protein through a non-natural side-by-side inter peptide linkage. In some aspects, CASTRs comprise a sequence from ATF4 that spans a defined region of basic and leucine zipper domain, and that is connected to a second monomer from the different CREB/P protein through a non-natural side-by-side inter peptide linkage

C. Methods for Design of High Affinity bZIP Transcriptional Repressors

Certain aspects herein provide design strategies for DNA-binding molecules. Certain aspects provide a design strategy which takes a bZIP transcription factor sequence and focuses on designing two individual monomeric polypeptides that may be modified, which will form DNA-binding molecules A and B. In some aspects, the DNA-binding molecules A and B will subsequently be covalently linked, such as through an interpeptide linker. This can create an A-B molecule. The molecule can be an adduct molecule. In some aspects, A and B independently do not bind DNA, and only designed A-B molecules will bind DNA. In some aspects, the A and B monomers contain one or more of the following structural/chemical features (i) to (iv):
i) In some aspects, a defined peptide footprint that may comprises approximately 19 amino acids at the C-terminus of the basic domain of a bZIP protein. In some aspects, the basic domain can be defined from the sequence. In some aspects, the defined peptide footprint comprises approximately 12 N-terminal residues of the leucine zipper portion of a bZIP transcription factor. In some aspects, each monomer A and B comprises a 31 amino acid peptide. In some aspects, the 31 amino acid peptide which can be truncated from either the N-terminus, the C-terminus, or both, by 1, 2, 3, 4, 5 6, 7, or more residues, or could be extended by 1, 2, 3, 4, 5 6, 7, or more, additional natural residues or non-natural residues from the the N-terminus, the C-terminus, or both. The peptide, in certain aspects, still has a defined structure of each A and B monomer, with a defined a junction between the basic and leucine zipper regions.
ii) In certain aspects, each A and B monomer contains one or more natural and/or non-natural functional groups (which may be reactive groups) within each peptide at one of several defined positions that contain complementary reactivity on the opposite monomer. For example, the reactive group in A will react with the reactive group in B. The reactivity may be based on a defined reaction sequence present within A or B. The reactivity may be based through lack of reactivity with any other functional group present within A or B. In some aspects, the reactivity allows such that when a completed monomer A and completed monomer B are reacted with one another in solution, or with one remaining on solid support and the other in solution, a new covalent linkage is formed between the reactive groups to make an A-B molecule. In some aspects, the A-B molecule is necessary and sufficient to bind DNA with high affinity, specificity and to stabilize the molecule for biological use. The location for the linkage can be determined generically for any bZIP based on basic/leucine zipper junction position. In some aspects, the reaction scheme is directionality agnostic, meaning the first functional group that reacts with a second functional group may be on A and the second functional group is on B. Conversely, the first functional group that reacts with a second functional group may be on B and the second functional group is on A.
iii) In some aspects, either or both the A and B monomer comprise helical stabilization chemistry. The helical stabilization chemistry may improve affinity, specificity and/or biological stability. In some aspects, the stabilizing chemistry comprises an intrapeptide stabilizing linkage. In some aspects, both monomers comprise stabilizing chemistry within each helix and both monomers comprise interpeptide linkage, all of which is compatible chemically. Certain aspects use ring closing metathesis, including at I, I+4 positioned amino acids. The amino acid may be an S5 amino acid. Certain aspects define several generic locations within each A and B monomers from any bZIP protein that could accommodate the individual side-chain amino acids, including S5. In some aspects, the stabilizing chemistry, such as incorporation of a stabilizing amino acid and/or intrapeptide stabilizing linkage, is incorporated while synthesizing the peptide monomers, then cyclized or connected. In some aspects, the stabilizing chemistry is incorporated while still synthesizing each individual monomer such that the original amino acids incorporated are now connected and stabilize. In some aspects, the stabilized molecules are further bound together (e.g., through adduction) to create the A-B molecule. Many stabilizing chemistries are possible, including but not limited to lactam connections, cross-coupling mediated C—C bond connections, thioethers, ethers, secondary or tertiary amines, ketone connections, triazole connections, dials-alder adducts, inverse electron demand diels-alder adducts and others. The basic helix containing a helix stabilizing chemistry can also contain other changes to increase stability or otherwise alter DNA binding, such as aminoisobutyric acid, D-amino acids and/or other natural/unnatural substitutions.
iv) In some aspects, non-natural changes from the native bZIP protein sequence comprising monomer A and/or B are introduced within the leucine zipper region of one or both of the monomer A and B peptides to alter the natural sequence and enable proper structure formation and activity. Such changes may be made during synthesis of the peptides, including prior to adduction. The changes may then persist in the adduct. In some aspects, the non-natural changes comprise Rule (1) and/or Rule (2)

Rule 1) Position a, which is one amino acid to the c-terminus of basic region/leucine zipper junction, in the monomer is mutated to leucine in monomer A and leucine in monomer B if the natural amino acid for the bZIP protein comprising monomer A or B at this position is an amino acid other than isoleucine, leucine, or valine. In some aspects, if the natural residue at position a in either monomer A or B is an isoleucine, then the amino acid in position a of the other monomer is also mutated to an isoleucine.

Rule 2) Position d, which is four amino acids to the c-terminus of basic/leucine zipper junction, should be mutated to leucine in monomer A and leucine in monomer B if the natural amino acid for the bZIP protein comprising monomer A or B at this position is an amino acid other than isoleucine, leucine, or valine. If the natural residue in either monomer A or B is an isoleucine, then the amino acid in position d of the other monomer is mutated to an isoleucine.

In some aspects, the non-natural changes only comprise Rule (1) and/or Rule (2). In some aspects, including where no changes were made to positions a and/or b, positions e, five amino acids to the c-terminus of the junction, in monomer A and position g′, seven amino acids to the c-terminus of the junction, in monomer B, is mutated to be a Gln/Gln or Arg/Glu pair. In some aspects, when positions b, c, and/or f (i.e., two, three, or six amino acids to the c-terminus of the junction respectively) are glycine in the native bZIP sequence, one, two, or all of the positions are mutated to alanine.

As disclosed herein, the inventors have developed a novel, general, and modular pipeline for design of high affinity bZIP transcriptional repressors starting from any bZIP protein. Accordingly, aspects of the disclosure are directed to methods for design and generation of bZIP transcriptional repressors having high DNA binding affinities (e.g., K_{D} less than 50 nM, 25 nM, 15 nM, 5 nM, or even less). Any bZIP protein(s) may be subject to the disclosed design process to generate high affinity bZIP transcriptional repressors. A method of the disclosure may comprise 1, 2, 3, 4, 5, 6, or all of the following steps:

1) Obtain amino acid sequence of bZIP protein(s)—Obtain the sequences of a first and second natural bZIP proteins involved in DNA binding. For homodimers, the first and second bZIP proteins are the same protein. For heterodimers, the first and second bZIP proteins are different proteins. Sequences may be obtained from any database; for example sequences may be obtained from The Universal Protein Resource (UniProt).

2) Identify the natural basic domain and natural leucine zipper domain for both sequences based on the leucine hepta-repeat—Identify the natural leucine zipper domain sequences by identifying repetitive leucine in every seven residues, plus three more residues into the N-terminus from the first leucine. Then identify the natural basic domain sequences by identifying the 26 residues towards the N-terminus next to the first residue of leucine zipper domain.

3) Identify the minimum necessary DNA recognition sequence—Identify the minimum necessary DNA recognition sequence by identifying the first 12 residues of the leucine zipper domain and the first 21 residues on the C-terminal end of the basic domain.

4) Mutate all cysteines—Mutate all cysteine residues in both sequences based on the following rules: If a cysteine is in the basic domain, replace it with a serine.

If a cysteine is at a b, c, or f position of the leucine zipper domain, replace it with an alanine. If a cysteine is at an a or d position of the leucine zipper domain, replace it with a leucine.

Methods for determination of a, b, c, d, e, f, and g positions of a leucine zipper domain are recognized in the art and include those described in, for example, Hakoshima, T. (2014). Leucine Zippers. In eLS, John Wiley & Sons, Ltd (Ed.) and Deppmann et al., Mol Biol Evol. 2006; 23(8):1480-1492, both incorporated by reference in their entirety.

5) Identify the optimal linker position—Identify the linker positions as the last residue of the basic domain on the first peptide and the first residue at an e position of the leucine zipper domain on the second peptide. Mutate the linker position on the first peptide to a cysteines and the linker position on the second peptide to Lys(mmt). During synthesis of the dimer, these two positions may be coupled using 2-(2,5-dioxo-2,5-dihydro-1H-pyrrol-1-yl)acetic acid.

6) Replace weaker interhelix contact residues—Replace certain “weaker” residues based on the following rules: If a residue at an a or d position of a leucine zipper domain is neither a leucine nor an isoleucine, replace it with a leucine.

If a residue at a gi position of the first peptide and a paired residue at an eᵢ₊₁ position of the second peptide are not either KE, EK, RE, ER, or QQ, replace the positions so that they are KE or RE (where the first letter indicates the residue at the gi position of the first peptide and the second letter indicates the cᵢ₊₁ position of the second peptide).

7) Identify intrapeptide stabilizing linkage positions—Identify the intrapeptide stabilizing linkage positions as either the forth residue and eighth residue from the N-terminus of the peptide or the 22nd residue and 26th residue from the N-terminus of the peptide. Replace the intrapeptide stabilizing linkage positions with (S)-2-(4′-pentenyl)alanine. During synthesis of the dimer, intrapeptide stabilizing linkage may be generated by ring closing metathesis.

It is specifically contemplated that any 1, 2, 3, 4, 5, 6, or more of the preceding steps may be excluded from aspects of the disclosure.

### II. Proteins

As used herein, a “protein” or “polypeptide” refers to a molecule comprising at least five amino acid residues. As used herein, a “peptide” refers to a molecule comprising at least three amino acid residues. As used herein, the term “wild-type” refers to the endogenous version of a molecule that occurs naturally in an organism. In some aspects, wild-type versions of a protein or polypeptide are employed, however, in many aspects of the disclosure, a modified protein or polypeptide is employed to generate an immune response. The terms described above may be used interchangeably. A “modified protein” or “modified polypeptide” “modified peptide” or a “variant” refers to a protein or polypeptide whose chemical structure, particularly its amino acid sequence, is altered with respect to the wild-type protein or polypeptide. In some aspects, a modified/variant protein or polypeptide has at least one modified activity or function (recognizing that proteins or polypeptides may have multiple activities or functions). It is specifically contemplated that a modified/variant protein or polypeptide may be altered with respect to one activity or function yet retain a wild-type activity or function in other respects.

Where a protein is specifically mentioned herein, it is in general a reference to a native (wild-type) or recombinant (modified) protein or, optionally, a protein in which any signal sequence has been removed. The protein may be isolated directly from the organism of which it is native, produced by recombinant DNA/exogenous expression methods, or produced by solid-phase peptide synthesis (SPPS) or other in vitro methods. In particular aspects, there are isolated nucleic acid segments and recombinant vectors incorporating nucleic acid sequences that encode a polypeptide (e.g., an antibody or fragment thereof). The term “recombinant” may be used in conjunction with a polypeptide or the name of a specific polypeptide, and this generally refers to a polypeptide produced from a nucleic acid molecule that has been manipulated in vitro or that is a replication product of such a molecule.

In certain aspects the size of a protein or polypeptide (wild-type or modified) may comprise, but is not limited to, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1200, 1300, 1400, 1500, 1750, 2000, 2250, 2500 amino acid residues or greater, and any range derivable therein, or derivative of a corresponding amino sequence described or referenced herein. It is contemplated that polypeptides may be mutated by truncation, rendering them shorter than their corresponding wild-type form, also, they might be altered by fusing or conjugating a heterologous protein or polypeptide sequence with a particular function (e.g., for targeting or localization, for enhanced immunogenicity, for purification purposes, etc.). As used herein, the term “domain” refers to any distinct functional or structural unit of a protein or polypeptide, and generally refers to a sequence of amino acids with a structure or function recognizable by one skilled in the art.

The polypeptides, proteins, or polynucleotides encoding such polypeptides or proteins of the disclosure may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (or any derivable range therein) or more variant amino acids or nucleic acid substitutions or be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107,108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 350 or more contiguous amino acids or nucleic acids, or any range derivable therein, of SEQ ID NOs:1-166.

In some aspects, the protein or polypeptide may comprise amino acids 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107,108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, or 350 (or any derivable range therein) of SEQ ID NOs:1-166.

In some aspects, the protein, polypeptide, or nucleic acid may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107,108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, or 350 (or any derivable range therein) contiguous amino acids of SEQ ID NOs:1-166.

In some aspects, the polypeptide, protein, or nucleic acid may comprise at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, or 350 (or any derivable range therein) contiguous amino acids of SEQ ID NOs:1-166 that are at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with one of SEQ ID NOS:1-166.

In some aspects there is a nucleic acid molecule or polypeptide starting at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, or 350 of any of SEQ ID NOS:1-166 and comprising at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106,107, 108, 109, 110, 111, 112, 113, 114, 115, 116, 117, 118, 119, 120, 121,122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, or 350 (or any derivable range therein) contiguous amino acids or nucleotides of any of SEQ ID NOS:1-166.

The nucleotide as well as the protein, polypeptide, and peptide sequences for various genes have been previously disclosed, and may be found in the recognized computerized databases. Two commonly used databases are the National Center for Biotechnology Information's Genbank and GenPept databases (on the World Wide Web at ncbi.nlm.nih.gov/) and The Universal Protein Resource (UniProt; on the World Wide Web at uniprot.org). The coding regions for these genes may be amplified and/or expressed using the techniques disclosed herein or as would be known to those of ordinary skill in the art.

It is contemplated that in compositions of the disclosure, there is between about 0.001 mg and about 10 mg of total polypeptide, peptide, and/or protein per ml. The concentration of protein in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml or more (or any range derivable therein).

1. bZIP proteins

Aspects of the present disclosure comprise bZIP proteins, modified bZIP proteins, peptides from bZIP proteins, domains from bZIP proteins, and synthetic molecules comprising modified sequences from bZIP proteins. As used herein, a “bZIP protein” (also referred to herein as a “bZIP-containing transcription factor,” “bZIP transcription factor,” or “zTF”) describes any protein containing a basic leucine zipper region (also referred to herein as a “DNA binding region” of a bZIP protein) comprising two domains: a “basic domain” (also “basic region”), capable of direct interaction of the bZIP protein with DNA, and a “leucine zipper domain,” (also “dimerization domain,” “leucine zipper region,” or “leucine zipper”), capable of dimerization with another bZIP protein. A bZIP protein may be a human (Homo sapiens) bZIP protein or may be a non-human bZIP protein. Various examples of bZIP proteins are recognized in the art and described in, for example, Miller M. Curr Protein Pept Sci. 2009; 10(3):244-269; Ramji D P, Foka P. Biochem J. 2002; 365(Pt 3):561-575; Wagner E F. Oncogene. 2001; 20(19):2334-2335; Hai T, Hartman M G. Gene. 2001; 273(1):1-11; Bailey D, O'Hare P. Antioxid Redox Signal. 2007; 9(12):2305-2321; Hunger S P, et al., Blood. 1996; 87(11):4607-4617; Blank V, Andrews N C. Trends Biochem Sci. 1997; 22(11):437-441; Motohashi H, et al., Gene. 2002; 294(1-2):1-12; all of which are incorporated herein by reference in their entirety.

Non-limiting examples of bZIP proteins are provided in Table 4. Any one or more of the bZIP proteins of Table 4 may be used in the compositions and methods of the present disclosure. Contemplated herein are engineered peptides comprising sequences of any one or more of the bZIP proteins of Table 4.

TABLE 4

| protein name | abbreviation |
|---|---|
| CREB/ATF bZIP | CREBZF |
| transcription factor | |
| Endoplasmic reticulum | NFE2L1 |
| membrane sensor NFE2L1 | |
| Basic leucine zipper | BATF |
| transcriptional factor | |
| ATF-like | |
| Transcription regulator | BACH2 |
| protein BACH2 | |
| Transcription regulator | BACH1 |
| protein BACH1 | |
| Neural retina-specific | NRL |
| leucine zipper protein | |
| Cyclic AMP-responsive | CREB3L4 |
| element-binding protein | |
| 3-like protein 4 | |
| Transcription factor MafG | MafG |
| Nuclear factor erythroid | NRF2 |
| 2-related factor 2 | |
| Nuclear factor interleukin- | NFIL3 |
| 3-regulated protein | |
| Transcription factor NF-E2 | NFE2 |
| 45 kDa subunit | |
| Transcription factor MafK | MafK |
| Cyclic AMP-responsive | CREB1 |
| element-binding protein 1 | |
| Cyclic AMP-responsive | CREB3L2 |
| element-binding protein | |
| 3-like protein 2 | |
| Hepatic leukemia factor | HLF |
| Proto-oncogene c-Fos | cFOS |
| Transcription factor Maf | Maf |
| Transcription factor MafF | MafF |
| Transcription factor AP-1 | cJUN |
| Transcription factor MafA | MafA |
| Cyclic AMP-dependent | ATF6a |
| transcription factor | |
| ATF-6 alpha | |
| cAMP-responsive element | CREM |
| modulator | |
| Cyclic AMP-responsive | CREB3 |
| element-binding protein 3 | |
| Cyclic AMP-dependent | ATF1 |
| transcription factor ATF-1 | |
| Basic leucine zipper | ATFL3 |
| transcriptional factor | |
| ATF-like 3 | |
| Cyclic AMP-dependent | ATF7 |
| transcription factor ATF-7 | |
| Cyclic AMP-dependent | ATF6b |
| transcription factor | |
| ATF-6 beta | |
| Cyclic AMP-responsive | CREB3L1 |
| element-binding protein | |
| 3-like protein 1 | |
| Cyclic AMP-responsive | CREB5 |
| element-binding protein 5 | |
| Basic leucine zipper | ATFL2 |
| transcriptional factor | |
| ATF-like 2 | |
| Cyclic AMP-responsive | CREB3L3 |
| element-binding protein | |
| 3-like protein 3 | |
| Cyclic AMP-dependent | ATF4 |
| transcription factor ATF-4 | |
| Cyclic AMP-dependent | ATF3 |
| transcription factor ATF-3 | |
| Fos-related antigen 1 | FRA1 |
| Protein fosB | FOSB |
| Cyclic AMP-dependent | ATF2 |
| transcription factor ATF-2 | |
| D site-binding protein | DBP |
| Jun dimerization protein 2 | JDP2 |
| Transcription factor jun-B | JUNB |
| Transcription factor jun-D | JUND |
| Fos-related antigen 2 | FRA2 |
| Transcription factor MafB | MAFB |
| X-box-binding protein 1 | XBP1 |
| Cyclic AMP-dependent | ATF5 |
| transcription factor ATF-5 | |
| CCAAT/enhancer-binding | CEBPd |
| protein delta | |
| CCAAT/enhancer-binding | CEBPy |
| protein gamma | |
| CCAAT/enhancer-binding | CEBPb |
| protein beta | |
| CCAAT/enhancer-binding | CEBPa |
| protein alpha | |
| CCAAT/enhancer-binding | CEBPe |
| protein epsilon | |
| CREB3 regulatory factor | CREBRF |
| DNA damage-inducible | DDIT3 |
| transcript 3 protein | |
| Thyrotroph embryonic factor | TEF |
| cAMP-responsive element- | CEBPL2 |
| binding protein-like 2 | |
| Nuclear factor erythroid | NRF3 |
| 2-related factor 3 | |
| NFIL3 like protein | NFILZ |

In some aspects, the bZIP protein is c-Fos. c-Fos (or “Fos”) is a bZIP transcription factor encoded by the FOS gene. An example human c-Fos protein sequence is provided as SEQ ID NO:3. The basic domain of human c-Fos is provided as SEQ ID NO:108. The leucine zipper domain of human c-Fos is provided as SEQ ID NO:109.

In some aspects, the bZIP protein is c-Jun. c-Jun (also “AP-1” or “AP1” or “Jun”) is a bZIP transcription factor encoded by the JUN gene. An example human c-Jun protein sequence is provided as SEQ ID NO:6. The basic domain of human c-Jun is provided as SEQ ID NO:110. The leucine zipper domain of human c-Jun is provided as SEQ ID NO:111.

In some aspects, the bZIP protein is XBPL. XBP1 (or “X-box-binding protein 1”) is a bZIP transcription factor encoded by the XBP1 gene. An example human XBP1 protein sequence is provided as SEQ ID NO:9. The basic domain of human XBP1 is provided as SEQ ID NO:114. The leucine zipper domain of human XBP1 is provided as SEQ ID NO:115.

In some aspects, the bZIP protein is ATF4. ATF4 (or “Activating transcription factor 4”; also “CREB-2”) is a bZIP transcription factor encoded by the ATF4 gene. An example human ATF4 protein sequence is provided as SEQ ID NO:12. The basic domain of human ATF4 is provided as SEQ ID NO:118. The leucine zipper domain of human ATF4 is provided as SEQ ID NO:119.

In some aspects, the bZIP protein is C/EBPβ. C/EBPβ (or “C/EBP beta”) is a bZIP transcription factor encoded by the CEBPB gene. An example human C/EBPβ protein sequence is provided as SEQ ID NO:15. The basic domain of human C/EBPβ is provided as SEQ ID NO:122. The leucine zipper domain of human C/EBPβ is provided as SEQ ID NO:123.

Various bZIP proteins are recognized in the art and contemplated herein. Certain non-limiting examples of bZIP proteins are described in, for example, Vinson et al., Biochim Biophys Acta. 2006; 1759(1-2):4-12, and Newman et al., Science. 2003; 300(5628):2097-2101, each incorporated herein by reference in its entirety.

bZIP proteins may form heterodimers or homodimers in the context of DNA binding. Example bZIP protein dimers contemplated herein are provided in Table 5 below.

TABLE 5

Fos/Jun
XBP1/XBP1 (XBP1 homodimer)
ATF4/ATF4 (ATF4 homodimer)
C/EBPβ/ATF4

### 2. HIF Proteins

Aspects of the present disclosure are related to one or more hypoxia-inducible factor (HIF) proteins. HIF proteins include, but are not limited to, HIF1α (also “HIF-1α”), HIF2α (also “HIF-2α”), HIF3α (also “HIF-3α”), and HIF10 (also “HIF-1β”). HIF proteins are transcription factors recognized as regulators of the cellular response to hypoxia. Certain aspects of the disclosure relate to one or more HIF protein target genes, i.e., genes whose expression is regulated by a HIF transcription factor (e.g., HIF-1). Disclosed, in some aspects, are compositions and methods useful in reducing expression of a HIF protein target gene.

### 3. Sequences

Example amino acid and nucleotide sequences for various polypeptides, peptides, and nucleic acids of the disclosure are provided in Table 6 below.

TABLE 6

| | | SEQ ID |
|---|---|---|
| Name | Sequence | NO |
| c-Fos modified Basic | IRRERNKMAAAKCRNRRREL | 1 |
| domain-1 | | |
| c-Fos modified leucine | TDTLQAETDQLE | 2 |
| zipper domain-1 | | |
| c-Fos full length | MMFSGFNADYEASSSRCSSASPAGDSLSYYHSPAD | 3 |
| | SFSSMGSPVNAQDFCTDLAVSSANFIPTVTAISTSP | |
| | DLQWLVQPALVSSVAPSQTRAPHPFGVPAPSAGA | |
| | YSRAGVVKTMTGGRAQSIGRRGKVEQLSPEEEEK | |
| | RRIRRERNKMAAAKCRNRRRELTDTLQAETDQLE | |
| | DEKSALQTEIANLLKEKEKLEFILAAHRPACKIPDD | |
| | LGFPEEMSVASLDLTGGLPEVATPESEEAFTLPLLN | |
| | DPEPKPSVEPVKSISSMELKTEPFDDFLFPASSRPSG | |
| | SETARSVPDMDLSGSFYAADWEPLHSGSLGMGPM | |
| | ATELEPLCTPVVTCTPSCTAYTSSFVFTYPEADSFPS | |
| | CAAAHRKGSSSNEPSSDSLSSPTLLAL | |
| c-Jun modified Basic | RKRMRNRIAASKCRKRKLER | 4 |
| domain-1 | | |
| c-Jun modified leucine | IARLEEKVKTLK | 5 |
| zipper domain-1 | | |
| c-Jun full length | MTAKMETTFYDDALNASFLPSESGPYGYSNPKILK | 6 |
| | QSMTLNLADPVGSLKPHLRAKNSDLLTSPDVGLL | |
| | KLASPELERLIIQSSNGHITTTPTPTQFLCPKNVTDE | |
| | QEGFAEGFVRALAELHSQNTLPSVTSAAQPVNGA | |
| | GMVAPAVASVAGGSGSGGFSASLHSEPPVYANLS | |
| | NFNPGALSSGGGAPSYGAAGLAFPAQPQQQQQPP | |
| | HHLPQQMPVQHPRLQALKEEPQTVPEMPGETPPLS | |
| | PIDMESQERIKAERKRMRNRIAASKCRKRKLERIA | |
| | RLEEKVKTLKAQNSELASTANMLREQVAQLKQKV | |
| | MNHVNSGCQLMLTQQLQTF | |
| XBP1 modified Basic | RRKLKNRVAAQTARDRKKAR | 7 |
| domain-1 | | |
| XBP1 modified leucine | MSELEQQVVDLE | 8 |
| zipper domain-1 | | |
| XBP1 full length | MVVVAAAPNPADGTPKVLLLSGQPASAAGAPAG | 9 |
| | QALPLMVPAQRGASPEAASGGLPQARKRQRLTHL | |
| | SPEEKALRRKLKNRVAAQTARDRKKARMSELEQQ | |
| | VVDLEEENQKLLLENQLLREKTHGLVVENQELRQ | |
| | RLGMDALVAEEEAEAKGNEVRPVAGSAESAALRL | |
| | RAPLQQVQAQLSPLQNISPWILAVLTLQIQSLISCW | |
| | AFWTTWTQSCSSNALPQSLPAWRSSQRSTQKDPV | |
| | PYQPPFLCQWGRHQPSWKPLMN | |
| ATF4 modified Basic | KKMEQNKTAATRYRQKKRAE | 10 |
| domain-1 | | |
| ATF4 modified leucine | QEALTGECKELE | 11 |
| zipper domain-1 | | |
| ATF4 full length | MTEMSFLSSEVLVGDLMSPFDQSGLGAEESLGLLD | 12 |
| | DYLEVAKHFKPHGFSSDKAKAGSSEWLAVDGLVS | |
| | PSNNSKEDAFSGTDWMLEKMDLKEFDLDALLGID | |
| | DLETMPDDLLTTLDDTCDLFAPLVQETNKQPPQTV | |
| | NPIGHLPESLTKPDQVAPFTFLQPLPLSPGVLSSTPD | |
| | HSFSLELGSEVDITEGDRKPDYTAYVAMIPQCIKEE | |
| | DTPSDNDSGICMSPESYLGSPQHSPSTRGSPNRSLP | |
| | SPGVLCGSARPKPYDPPGEKMVAAKVKGEKLDKK | |
| | LKKMEQNKTAATRYRQKKRAEQEALTGECKELE | |
| | KKNEALKERADSLAKEIQYLKDLIEEVRKARGKK | |
| | RVP | |
| C/EBPbeta modified Basic | IRRERNNIAVRKSRDKAKMR | 13 |
| domain-1 | | |
| C/EBPbeta modified | NLETQHKVLELT | 14 |
| leucine zipper domain-1 | | |
| C/EBPbeta full length | MQRLVAWDPACLPLPPPPPAFKSMEVANFYYEAD | 15 |
| | CLAAAYGGKAAPAAPPAARPGPRPPAGELGSIGDH | |
| | ERAIDFSPYLEPLGAPQAPAPATATDTFEAAPPAPA | |
| | PAPASSGQHHDFLSDLFSDDYGGKNCKKPAEYGY | |
| | VSLGRLGAAKGALHPGCFAPLHPPPPPPPPPAELKA | |
| | EPGFEPADCKRKEEAGAPGGGAGMAAGFPYALRA | |
| | YLGYQAVPSGSSGSLSTSSSSSPPGTPSPADAKAPP | |
| | TACYAGAAPAPSQVKSKAKKTVDKHSDEYKIRRE | |
| | RNNIAVRKSRDKAKMRNLETQHKVLELTAENERL | |
| | QKKVEQLSRELSTLRNLFKQLPEPLLASSGHC | |
| c-Fos modified Basic | IRRERNKMAAAKSRNRRREC | 16 |
| domain-2 | | |
| c-Fos modified Basic | IRR#RNK#AAAKSRNRRREC | 17 |
| domain-3 | | |
| c-Fos modified Basic | EEKRRIRRERNKMAAAKSRNRRREC | 18 |
| domain-4 | | |
| c-Fos modified Basic | EEKRRIRR#RNK#AAAKSRNRRREC | 19 |
| domain-5 | | |
| c-Fos modified leucine | TDTLEDETDQLE | 20 |
| zipper domain-2 | | |
| c-Fos modified leucine | LDELQAEIEQLE | 21 |
| zipper domain-3 | | |
| c-Fos modified leucine | IDELQAEIEQLE | 22 |
| zipper domain-4 | | |
| c-Fos modified leucine | IDEIQAEIEQIE | 23 |
| zipper domain-5 | | |
| c-Fos modified leucine | L#ELQ#EIEQLE | 24 |
| zipper domain-6 | | |
| c-Fos modified leucine | I#ELQ#EIEQLE | 25 |
| zipper domain-7 | | |
| c-Fos modified leucine | I#EIQ#EIEQIE | 26 |
| zipper domain-8 | | |
| c-Jun modified Basic | RKRMRNRIAASKSRKRKLER | 27 |
| domain-2 | | |
| c-Jun modified Basic | RKR#RNR#AASKSRKRKLER | 28 |
| domain-3 | | |
| c-Jun modified Basic | RIKAERKRMRNRIAASKSRKRKLER | 29 |
| domain-4 | | |
| c-Jun modified Basic | RIKAERKR#RNR#AASKSRKRKLER | 30 |
| domain-5 | | |
| c-Jun modified leucine | IA*LEEKVKTLK | 31 |
| zipper domain-2 | | |
| c-Jun modified leucine | IARL*EKVKTLK | 32 |
| zipper domain-3 | | |
| c-Jun modified leucine | AAEL*EKVATLK | 33 |
| zipper domain-4 | | |
| c-Jun modified leucine | IARL*EKIKTLK | 34 |
| zipper domain-5 | | |
| c-Jun modified leucine | IARI*EKIKTIK | 35 |
| zipper domain-6 | | |
| c-Jun modified leucine | I#RL*#KVKTLK | 36 |
| zipper domain-7 | | |
| c-Jun modified leucine | I#RL*#KIKTLK | 37 |
| zipper domain-8 | | |
| XBP1 modified Basic | RRKLKNRVAAQTARDRKKAC | 38 |
| domain-2 | | |
| XBP1 modified Basic | RRK#KNR#AAQTARDRKKAC | 39 |
| domain-3 | | |
| XBP1 modified leucine | MSELEQQVVDLE | 40 |
| zipper domain-2 | | |
| XBP1 modified leucine | LSELEQQVVDLE | 41 |
| zipper domain-3 | | |
| XBP1 modified leucine | L#ELE#QVVDLE | 42 |
| zipper domain-4 | | |
| XBP1 modified Basic | RRKLKNRVAAQTARDRKKAR | 43 |
| domain-4 | | |
| XBP1 modified Basic | RRK#KNR#AAQTARDRKKAR | 44 |
| domain-5 | | |
| XBP1 modified leucine | MSEL*QQVVDLE | 45 |
| zipper domain-5 | | |
| XBP1 modified leucine | LSEL*QQVVDLE | 46 |
| zipper domain-6 | | |
| XBP1 modified leucine | L#EL*#QVVDLE | 47 |
| zipper domain-7 | | |
| ATF4 modified Basic | KKMEQNKTAATRYRQKKRAC | 48 |
| domain-2 | | |
| ATF4 modified Leucine | QEALTGELKELE | 49 |
| Zipper domain-2 | | |
| ATF4 modified Leucine | LEALKAELKELR | 50 |
| Zipper domain-3 | | |
| ATF4 modified Leucine | L#ALK#ELKELR | 51 |
| Zipper domain-4 | | |
| C/EBPbeta modified Basic | IRRERNNIAVRKSRDKAKMC | 52 |
| domain-2 | | |
| C/EBPbeta modified Basic | LLELQHKVLELR | 53 |
| domain-3 | | |
| C/EBPbeta modified Basic | L#ELQ#KVLELR | 54 |
| domain-4 | | |
| ATF4 modified Basic | KKMEQNKTAATRYRQKKRAE | 55 |
| domain-3 | | |
| ATF4 modified Leucine | QEAL*GELKELE | 56 |
| Zipper domain-5 | | |
| ATF4 modified Leucine | LEAL*AELKELR | 57 |
| Zipper domain-6 | | |
| ATF4 modified Leucine | L#AL*#ELKELR | 58 |
| Zipper domain-7 | | |
| Full Engineered c-Fos-1 | IRRERNKMAAAKSRNRRRECTDTLEDETDQLE | 59 |
| Full Engineered c-Fos-2 | IRRERNKMAAAKSRNRRRECLDELQAEIEQLE | 60 |
| Full Engineered c-Fos-3 | IRRERNKMAAAKSRNRRRECIDELQAEIEQLE | 61 |
| Full Engineered c-Fos-4 | IRRERNKMAAAKSRNRRRECIDEIQAEIEQIE | 62 |
| Full Engineered c-Fos-5 | IRR#RNK#AAAKSRNRRRECLDELQAEIEQLE | 63 |
| Full Engineered c-Fos-6 | IRRERNKMAAAKSRNRRRECL#ELQ#EIEQLE | 64 |
| Full Engineered c-Fos-7 | IRR#RNK#AAAKSRNRRRECIDELQAEIEQLE | 65 |
| Full Engineered c-Fos-8 | IRRERNKMAAAKSRNRRRECI#ELQ#EIEQLE | 66 |
| Full Engineered c-Fos-9 | IRR#RNK#AAAKSRNRRRECIDEIQAEIEQIE | 67 |
| Full Engineered c-Fos-10 | IRRERNKMAAAKSRNRRRECI#EIQ#EIEQIE | 68 |
| Full Engineered c-Fos-11 | EEKRRIRRERNKMAAAKSRNRRRECLDELQAEIEQ | 69 |
| | LE | |
| Full Engineered c-Fos-12 | EEKRRIRR#RNK#AAAKSRNRRRECLDELQAEIEQL | 70 |
| | E | |
| Full Engineered c-Fos-13 | EEKRRIRRERNKMAAAKSRNRRRECL#ELQ#EIEQ | 71 |
| | LE | |
| Full Engineered c-Jun-1 | RKRMRNRIAASKSRKRKLERIA*LEEKVKTLK | 72 |
| Full Engineered c-Jun-2 | RKRMRNRIAASKSRKRKLERIARL*EKVKTLK | 73 |
| Full Engineered c-Jun-3 | RKRMRNRIAASKSRKRKLERAAEL*EKVATLK | 74 |
| Full Engineered c-Jun-4 | RKRMRNRIAASKSRKRKLERIARL*EKIKTLK | 75 |
| Full Engineered c-Jun-5 | RKRMRNRIAASKSRKRKLERIARI*EKIKTIK | 76 |
| Full Engineered c-Jun-6 | RKR#RNR#AASKSRKRKLERIARL*EKVKTLK | 77 |
| Full Engineered c-Jun-7 | RKRMRNRIAASKSRKRKLERI#RL*#KVKTLK | 78 |
| Full Engineered c-Jun-8 | RKR#RNR#AASKSRKRKLERIARL*EKIKTLK | 79 |
| Full Engineered c-Jun-9 | RKRMRNRIAASKSRKRKLERI#RL*#KIKTLK | 80 |
| Full Engineered c-Jun-10 | RIKAERKRMRNRIAASKSRKRKLERIARL*EKVKT | 81 |
| | LK | |
| Full Engineered c-Jun-11 | RIKAERKR#RNR#AASKSRKRKLERIARL*EKVKTL | 82 |
| | K | |
| Full Engineered c-Jun-12 | RIKAERKRMRNRIAASKSRKRKLERI#RL*#KVKTL | 83 |
| | K | |
| Full Engineered XBP1 | RRKLKNRVAAQTARDRKKACMSELEQQVVDLE | 84 |
| First-1 | | |
| Full Engineered XBP1 | RRKLKNRVAAQTARDRKKACLSELEQQVVDLE | 85 |
| First-2 | | |
| Full Engineered XBP1 | RRKLKNRVAAQTARDRKKACL#ELE#QVVDLE | 86 |
| First-3 | | |
| Full Engineered XBP1 | RRK#KNR#AAQTARDRKKACLSELEQQVVDLE | 87 |
| First-4 | | |
| Full Engineered XBP1 | RRKLKNRVAAQTARDRKKARMSEL*QQVVDLE | 88 |
| Second-1 | | |
| Full Engineered XBP1 | RRKLKNRVAAQTARDRKKARLSEL*QQVVDLE | 89 |
| Second-2 | | |
| Full Engineered XBP1 | RRKLKNRVAAQTARDRKKARL#EL*#QVVDLE | 90 |
| Second-3 | | |
| Full Engineered XBP1 | RRK#KNR#AAQTARDRKKARLSEL*QQVVDLE | 91 |
| Second-4 | | |
| Full Engineered ATF4 | KKMEQNKTAATRYRQKKRACQEALTGELKELE | 92 |
| First-1 | | |
| Full Engineered ATF4 | KKMEQNKTAATRYRQKKRACLEALKAELKELR | 93 |
| First-2 | | |
| Full Engineered ATF4 | KKMEQNKTAATRYRQKKRACL#ALK#ELKELR | 94 |
| First-3 | | |
| Full Engineered ATF4 | KKMEQNKTAATRYRQKKRAEQEAL*GELKELE | 95 |
| Second-1 | | |
| Full Engineered ATF4 | KKMEQNKTAATRYRQKKRAELEAL*AELKELR | 96 |
| Second-2 | | |
| Full Engineered ATF4 | KKMEQNKTAATRYRQKKRAEL#AL*#ELKELR | 97 |
| Second-3 | | |
| Full Engineered | IRRERNNIAVRKSRDKAKMCLLELQHKVLELR | 98 |
| C/EBPbeta First-1 | | |
| Full Engineered | IRRERNNIAVRKSRDKAKMCL#ELQ#KVLELR | 99 |
| C/EBPbeta First-2 | | |
| UPRE | CGCTTGATGACGTGGCCGGAA | 100 |
| AP-1 | CGCTTGATGACTCAGCCGGAA | 101 |
| Consensus | CGCTTGATGACGTGGCCGGAA | 102 |
| Mutant | CGCTTGATGAAGGGGCCGGAA | 103 |
| c-Fos full Basic + leucine | MKRRIRRERNKMAAAKCRNRRRELTDTLQAETDQ | 104 |
| zipper | LEDEKSALQTEIANLLKEKEKLEFILAAH | |
| c-Jun full Basic + leucine | ERIKAEKRKMRNRIAASKCRKRKLERIARLEEKVK | 105 |
| zipper | TLKAQNSELASTANMLREQVAQLKQKVMN | |
| c-Fos minimum | IRRERNKMAAAKCRNRRRELTDTLQAETDQLE | 106 |
| recognition sequence | | |
| c-Jun minimum | KRKMRNRIAASKCRKRKLERIARLEEKVKTLK | 107 |
| recognition sequence | | |
| c-Fos natural Basic | MKRRIRRERNKMAAAKCRNRRREL | 108 |
| domain | | |
| c-Fos natural leucine | TDTLQAETDQLEDEKSALQTEIANLLKEKEKLEFIL | 109 |
| zipper domain | AAH | |
| c-Jun natural Basic | ERIKAEKRKMRNRIAASKCRKRKLER | 110 |
| Domain | | |
| c-Jun natural leucine | IARLEEKVKTLKAQNSELASTANMLREQVAQLKQ | 111 |
| zipper domain | KVMN | |
| CREBZF minimum | SPRKAAAAAARLNRLKKKEYVMGLESRVRGLA | 112 |
| recognition sequence | | |
| NFE2L1 engineered | RRRGKNKMAAQNSRKRKLDTILNLERDVEDLQ | 113 |
| peptide | | |
| BATF engineered peptide | QRREKNRIAAQKCRQRQTQKADTLHLESEDLE | 114 |
| BACH2 engineered | RRRSKNRIAAQRCRKRKLDCIQNLESEIRKLV | 115 |
| peptide | | |
| BACH1 engineered | RRRSKNRIAAQRCRKRKLDCIQNLESEIEKLQ | 116 |
| peptide | | |
| NRL engineered peptide | RRTLKNRGYAQACRSKRLQQRRGLEAERARLA | 117 |
| CREB3L4 engineered | RRKKEYIDGLESRVAACSAQNQELQKKVQELE | 118 |
| peptide | | |
| MafG engineered peptide | RRTLKNRGYAASCRVKRVTQKEELEKQKAELQ | 119 |
| NRF2 engineered peptide | RRRGKNKVAAQNCRKRKLENIVELEQDLDHLK | 120 |
| NFIL3 engineered peptide | EKRRKNNEAAKRSREKRRLNDLVLENKLIALG | 121 |
| NFE2 engineered peptide | RRRGKNKVAAQNCRKRKLETIVQLERELERLT | 122 |
| MafK engineered peptide | RRTLKNRGYAASCRIKRVTQKEELERQRVELQ | 123 |
| CREB1 engineered | VRLMKNREAARESRRKKKEYVKSLENRVAVLE | 124 |
| peptide | | |
| CREB3L2 engineered | RKKKEYMDSLEKKVESCSTENLELRKKVEVLE | 125 |
| peptide | | |
| HLF engineered peptide | MAAKRSRDARRLKENQIAIRASFLEKENSALR | 126 |
| cFOS engineered peptide | IRRERNKMAAAKCRNRRRELTDTLQAETDQLE | 127 |
| Maf engineered peptide | RRTLKNRGYAQSCRFKRVQQRHVLESEKNQLL | 128 |
| MafF engineered peptide | RRTLKNRGYAASCRVKRVSQKEELQKQKSELE | 129 |
| cJUN engineered peptide | RKRMRNRIAASKSRKRKLERIARLEEKVKTLK | 130 |
| MafA engineered peptide | RRTLKNRGYAQSSRFKRVQQRHILESEKCQLQ | 131 |
| ATF6a engineered peptide | KKKKEYMLGLEARLKAALSENEQLKKENGTLK | 132 |
| CREM engineered peptide | LRLMKNREAAKESRRRKKEYVKSLESRVAVLE | 133 |
| CREB3 engineered | RKKKVYVGGLESRVLKYTAQNMELQNKVQLL | 134 |
| peptide | | |
| ATF1 engineered peptide | IRLMKNREAARECRRKKKEYVKSLENRVAVLE | 135 |
| ATFL3 engineered peptide | RRREKNRVAAQRSRKKQTQKADKLHEEYESLE | 136 |
| ATF7 engineered peptide | RFLERNRAAASRSRQKRKLWVSSLEKKAEELT | 137 |
| ATF6b engineered peptide | RQQRMIKNRESASQSRRKKKEYLQGLEARLQ | 138 |
| CREB3L1 engineered | RKKKEYVESLEKKVETFTSENNELWKKVETLE | 139 |
| peptide | | |
| CREB5 engineered | KFLERNRAAATRSRQKRKVWVMSLEKKAEELT | 140 |
| peptide | | |
| ATFL2 engineered peptide | LKKQKNRAAAQRSRQKHTDKADALHQQHESLE | 141 |
| CREB3L3 engineered | KKKKEYIDGLETRMSASTAQNQELQRKVLHLE | 142 |
| peptide | | |
| ATF4 engineered peptide | KKMEQNKTAATRYRQKKRAEQEALTGESKELE | 143 |
| ATF3 engineered peptide | RRRERNKIAAAKSRNKKKEKTESLQKESEKLE | 144 |
| FRA1 engineered peptide | RRERNKLAAAKSRNRRKELTDFLQAETDKLE | 145 |
| FOSB engineered peptide | VRRERNKLAAAKSRNRRRELTDRLQAETDQLE | 146 |
| ATF2 engineered peptide | KFLERNRAAASRSRQKRKVWVQSLEKKAEDLS | 147 |
| DBP engineered peptide | SRRYKNNEAAKRSRDARRLKENQISVRAAFLE | 148 |
| JDP2 engineered peptide | RRREKNKVAAARSRNKKKERTEFLQRESERLE | 149 |
| JUNB engineered peptide | RKRLRNRLAATKSRKRKLERIARLEDKVKTLK | 150 |
| JUND engineered peptide | RKRLRNRIAASKSRKRKLERISRLEKVKTLK | 151 |
| FRA2 engineered peptide | IGTTVGRRRRDEQLSPEEEEKRRIRRERNKLA | 152 |
| MAFB engineered peptide | RRTLKNRGYAQSSRYKRVQQKHHLENEKTQLI | 153 |
| XBP1 engineered peptide | RRKLKNRVAAQTARDRKKARMSELEQQVVDLE | 154 |
| ATF5 engineered peptide | KKRDQNKSAALRYRQRKRAEGEALEGESQGLE | 155 |
| CEBPd engineered peptide | IAVRKSRDKAKRRNQEMQQKLVELSAENEKLH | 156 |
| CEBPy engineered peptide | MAVKKSRLKSKQKAQDTLQRVNQLKEENERLE | 157 |
| CEBPb engineered peptide | YKIRRERNNIAVRKSRDKAKMRNLETQHKVLE | 158 |
| CEBPa engineered peptide | IAVRKSRDKAKQRNVETQQKVLELTSDNDRLR | 159 |
| CEBPe engineered peptide | YRLRRERNNIAVRKSRDKAKRRILETQQKVLE | 160 |
| CREBRF engineered | SDLTPVSELPLTARPRSRKEKNKLASRASRLK | 161 |
| peptide | | |
| DDIT3 engineered peptide | SPARAGKQRMKEKEQENERKVAQLAEENERLK | 162 |
| TEF engineered peptide | DEKYWTRRKKNNVAAKRSRDARRLKENQITIR | 163 |
| CEBPL2 engineered | ESRARKKLRYQYLEELVSSRERAISALREELE | 164 |
| peptide | | |
| NRF3 engineered peptide | RRRGKNKVAAQNSRKRKLDIILNLEDDVSNLQ | 165 |
| NFILZ engineered peptide | EKRRKNNEAAKRSREKRRLNDAAIEGRLAALM | 166 |

# = (S)-2-(4′-pentenyl)alanine
* = Lys(Mmt)

### 4. Variant Polypeptides

The following is a discussion of changing the amino acid subunits of a protein or peptide to create an equivalent, or even improved, variant polypeptide or peptide. Since it is the interactive capacity and nature of a protein that defines that protein's functional activity, certain amino acid substitutions can be made in a protein or peptide sequence, and nevertheless produce a protein with similar or more desirable properties.

The term “functionally equivalent codon” is used herein to refer to codons that encode the same amino acid, such as the six different codons for arginine. Also considered are “neutral substitutions” or “neutral mutations” which refers to a change in the codon or codons that encode biologically equivalent amino acids.

Amino acid sequence variants of the disclosure can be substitutional, insertional, or deletion variants. A variation in a polypeptide of the disclosure may affect 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more non-contiguous or contiguous amino acids of the protein or polypeptide, as compared to wild-type. A variant can comprise an amino acid sequence that is at least 50%, 60%, 70%, 80%, or 90%, including all values and ranges there between, identical to any sequence provided or referenced herein. A variant can include 2, 3, 4, 5, 6, 7, 8, 9, 10, 11, 12, 13, 14, 15, 16, 17, 18, 19, 20, or more substitute amino acids.

It also will be understood that amino acid and nucleic acid sequences may include additional residues, such as additional N- or C-terminal amino acids, or 5′ or 3′ sequences, respectively, and yet still be essentially identical as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

Deletion variants typically lack one or more residues of the native or wild type protein. Individual residues can be deleted or a number of contiguous amino acids can be deleted. A stop codon may be introduced (by substitution or insertion) into an encoding nucleic acid sequence to generate a truncated protein.

Insertional mutants typically involve the addition of amino acid residues at a non-terminal point in the polypeptide. This may include the insertion of one or more amino acid residues. Terminal additions may also be generated and can include fusion proteins which are multimers or concatemers of one or more peptides or polypeptides described or referenced herein.

Substitutional variants typically contain the exchange of one amino acid for another at one or more sites within the protein or polypeptide, and may be designed to modulate one or more properties of the polypeptide, with or without the loss of other functions or properties. Substitutions may be conservative, that is, one amino acid is replaced with one of similar chemical properties. “Conservative amino acid substitutions” may involve exchange of a member of one amino acid class with another member of the same class. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Conservative amino acid substitutions may encompass non-naturally occurring amino acid residues, which are typically incorporated by chemical peptide synthesis rather than by synthesis in biological systems. These include peptidomimetics or other reversed or inverted forms of amino acid moieties.

Alternatively, substitutions may be “non-conservative”, such that a function or activity of the polypeptide is affected. Non-conservative changes typically involve substituting an amino acid residue with one that is chemically dissimilar, such as a polar or charged amino acid for a nonpolar or uncharged amino acid, and vice versa. Non-conservative substitutions may involve the exchange of a member of one of the amino acid classes for a member from another class.

### 5. Considerations for Substitutions

One skilled in the art can determine suitable variants of polypeptides as set forth herein using well-known techniques. One skilled in the art may identify suitable areas of the molecule that may be changed without destroying activity by targeting regions not believed to be important for activity. The skilled artisan will also be able to identify amino acid residues and portions of the molecules that are conserved among similar proteins or polypeptides. In further aspects, areas that may be important for biological activity or for structure may be subject to conservative amino acid substitutions without significantly altering the biological activity or without adversely affecting the protein or polypeptide structure.

In making such changes, the hydropathy index of amino acids may be considered. The hydropathy profile of a protein is calculated by assigning each amino acid a numerical value (“hydropathy index”) and then repetitively averaging these values along the peptide chain. Each amino acid has been assigned a value based on its hydrophobicity and charge characteristics. They are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (1.6); histidine (−3.2); glutamate (−3.5); glutamine (−3.5); aspartate (−3.5); asparagine (−3.5); lysine (−3.9); and arginine (−4.5). The importance of the hydropathy amino acid index in conferring interactive biologic function on a protein is generally understood in the art (Kyte et al., J. Mol. Biol. 157:105-131 (1982)). It is accepted that the relative hydropathic character of the amino acid contributes to the secondary structure of the resultant protein or polypeptide, which in turn defines the interaction of the protein or polypeptide with other molecules, for example, enzymes, substrates, receptors, DNA, and others. It is also known that certain amino acids may be substituted for other amino acids having a similar hydropathy index or score, and still retain a similar biological activity. In making changes based upon the hydropathy index, in certain aspects, the substitution of amino acids whose hydropathy indices are within ±2 is included. In some aspects of the present disclosure, those that are within ±1 are included, and in other aspects of the present disclosure, those within ±0.5 are included.

Additionally, one skilled in the art can review structure-function studies identifying residues in similar polypeptides or proteins that are important for activity or structure. In view of such a comparison, one can predict the importance of amino acid residues in a protein that correspond to amino acid residues important for activity or structure in similar proteins. One skilled in the art may opt for chemically similar amino acid substitutions for such predicted important amino acid residues.

### 6. Amino Acids

The term “amino acid” refers to natural amino acids, non-natural amino acids (also “unnatural amino acids”), and amino acid analogs, all in their D and L stereoisomers, unless otherwise indicated, if their structures allow such stereoisomeric forms. An amino acid, may be e.g., of the formula:

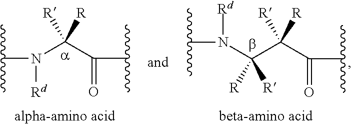

wherein each instance of R and R′ independently are selected from the group consisting of hydrogen, optionally substituted aliphatic, optionally substituted heteroaliphatic, optionally substituted aryl, optionally substituted heteroaryl, and R^{d} is hydrogen or an amino protecting group. Amino acids encompassed by the above two formulae include, without limitation, natural alpha-amino acids such as D- and L-isomers of the 20 common naturally occurring alpha-amino acids found in polypeptides and proteins (e.g., A, R, N, C, D, Q, E, G, H, I, L, K, M, F, P, S, T, W, Y, V, as depicted in Table 7 below), non-natural alpha-amino acids (examples of which are depicted in Table 8 below), natural beta-amino acids (e.g., beta-alanine), and unnatural beta-amino acids.

TABLE 7
Natural Amino Acids

| Amino Acid | R | R′ |
|---|---|---|
| L-Alanine (A) | —CH₃ | —H |
| L-Arginine (R) | —CH₂CH₂CH₂—NHC(═NH)NH₂ | —H |
| L-Asparagine (N) | —CH₂C(═O)NH₂ | —H |
| L-Aspartic acid (D) | —CH₂CO₂H | —H |
| L-Cysteine (C) | —CH₂SH | —H |
| L-Glutamic acid (E) | —CH₂CH₂CO₂H | —H |
| L-Glutamine (Q) | —CH₂CH₂C(═O)NH₂ | —H |
| Glycine (G) | —H | —H |
| L-Histidine (H) | —CH₂-2-(1H-imidazole) | —H |
| L-Isoleucine (I) | -sec-butyl | —H |
| L-Leucine (L) | -iso-butyl | —H |
| L-Lysine (K) | —CH₂CH₂CH₂CH₂NH₂ | —H |
| L-Methionine (M) | —CH₂CH₂SCH₃ | —H |
| L-Phenylalanine (F) | —CH₂Ph | —H |
| L-Proline (P) | -2-(pyrrolidine) | —H |
| L-Serine (S) | —CH₂OH | —H |
| L-Threonine (T) | —CH₂CH(OH)(CH₃) | —H |
| L-Tryptophan (W) | —CH₂-3-(1H-indole) | —H |
| L-Tyrosine (Y) | —CH₂-(p-hydroxyphenyl) | —H |
| L-Valine (V) | -isopropyl | —H |

TABLE 8
Non-natural Amino Acids

| Amino Acid | R | R′ |
|---|---|---|
| D-Alanine | —H | —CH₃ |
| D-Arginine | —H | —CH₂CH₂CH₂—NHC(═NH)NH₂ |
| D-Asparagine | —H | —CH₂C(═O)NH₂ |
| D-Aspartic acid | —H | —CH₂CO₂H |
| D-Cysteine | —H | —CH₂SH |
| D-Glutamic acid | —H | —CH₂CH₂CO₂H |
| D-Glutamine | —H | —CH₂CH₂C(═O)NH₂ |
| D-Histidine | —H | —CH₂-2-(1H-imidazole) |
| D-Isoleucine | —H | -sec-butyl |
| D-Leucine | —H | -iso-butyl |
| D-Lysine | —H | —CH₂CH₂CH₂CH₂NH₂ |
| D-Methionine | —H | —CH₂CH₂SCH₃ |
| D-Phenylalanine | —H | —CH₂Ph |
| D-Proline | —H | -2-(pyrrolidine) |
| D-Serine | —H | —CH₂OH |
| D-Threonine | —H | —CH₂CH(OH)(CH₃) |
| D-Tryptophan | —H | —CH₂-3-(1H-indole) |
| D-Tyrosine | —H | —CH₂-(p-hydroxyphenyl) |
| D-Valine | —H | -isopropyl |
| Di-vinyl | —CH═CH₂ | —CH═CH₂ |

| Amino Acid | R and R′ are equal to: |
|---|---|
| α-methyl-Alanine (Aib) | —CH₃, —CH₃ |
| α-methyl-Arginine | —CH₃, —CH₂CH₂CH₂—NHC(═NH)NH₂ |
| α-methyl-Asparagine | —CH₃, —CH₂C(═O)NH₂ |
| α-methyl-Aspartic acid | —CH₃, —CH₂CO₂H |
| α-methyl-Cysteine | —CH₃, —CH₂SH |
| α-methyl-Glutamic acid | —CH₃, —CH₂CH₂CO₂H |
| α-methyl-Glutamine | —CH₃, —CH₂CH₂C(═O)NH₂ |
| α-methyl-Histidine | —CH₃, —CH₂-2-(1H-imidazole) |
| α-methyl-Isoleucine | —CH₃, -sec-butyl |
| α-methyl-Leucine | —CH₃, -iso-butyl |
| α-methyl-Lysine | —CH₃, —CH₂CH₂CH₂CH₂NH₂ |
| α-methyl-Methionine | —CH₃, —CH₂CH₂SCH₃ |
| α-methyl-Phenylalanine | —CH₃, —CH₂Ph |
| α-methyl-Proline | —CH₃, -2-(pyrrolidine) |
| α-methyl-Serine | —CH₃, —CH₂OH |
| α-methyl-Threonine | —CH₃, —CH₂CH(OH)(CH₃) |
| α-methyl-Tryptophan | —CH₃, —CH₂-3-(1H-indole) |
| α-methyl-Tyrosine | —CH₃, —CH₂-(p-hydroxyphenyl) |
| α-methyl-Valine | —CH₃, -isopropyl |
| Di-vinyl Norleucine | —CH═CH₂, —CH═CH₂, —H, |
| | —CH₂CH₂CH₂CH₃ |

There are many known unnatural amino acids any of which may be included in the polypeptides of the present invention. See, for example, S. Hunt, The Non-Protein Amino Acids: In Chemistry and Biochemistry of the Amino Acids, edited by G. C. Barrett, Chapman and Hall, 1985; incorporated by reference in its entirety. Some examples of unnatural amino acids are 4-hydroxyproline, desmosine, gamma-aminobutyric acid, beta-cyanoalanine, norvaline, 4-(E)-butenyl-4(R)-methyl-N-methyl-L-threonine, N-methyl-L-leucine, 1-amino-cyclopropanecarboxylic acid, 1-amino-2-phenyl-cyclopropanecarboxylic acid, 1-amino-cyclobutanecarboxylic acid, 4-amino-cyclopentenecarboxylic acid, 3-amino-cyclohexanecarboxylic acid, 4-piperidylacetic acid, 4-amino-1-methylpyrrole-2-carboxylic acid, 2,4-diaminobutyric acid, 2,3-diaminopropionic acid, 2,4-diaminobutyric acid, 2-aminoheptanedioic acid, 4-(aminomethyl)benzoic acid, 4-aminobenzoic acid, ortho-, meta- and para-substituted phenylalanines (e.g., substituted with —C(═O)C₆H₅; —CF₃; —CN; -halo; —NO₂; —CH₃), disubstituted phenylalanines, substituted tyrosines (e.g., further substituted with —C(═O)C₆H₅; —CF₃; —CN; -halo; —NO₂; —CH₃), and statine.

Certain unnatural amino acids may be included in a polypeptide chain for peptide stapling or stitching. These unnatural amino acids include a terminal unsaturated moiety, such as a double or triple bond. Exemplary amino acids with terminal olefinic unsaturation include, but are not limited to, —(CH₂)_{g}—S—(CH₂)_{g}CH═CH₂; —(CH₂)_{g}—O—(CH₂)_{g}CH═CH₂; —(CH₂)_{g}—NH—(CH₂)_{g}CH═CH₂; —(CH₂)_{g}—(C═O)—S(CH₂)_{g}CH═CH₂; —(CH₂)_{g} (C═O)—O—(CH₂)_{g}CH═CH₂; —(CH₂)_{g}—(C═O) NH (CH₂)_{g}CH═CH₂; —CH₂CH₂CH₂CH₂—NH—(CH₂)_{g}CH═CH₂; (C₆H₅)-p-O—(CH₂)_{g}CH═CH₂; —CH(CH₃)—O—(CH₂)_{g}CH═CH₂; —CH₂CH(—O—CH═CH₂)(CH₃); -histidine-N((CH₂)_{g}CH═CH₂); -tryptophan-N((CH₂)_{g}CH═CH₂); and (CH₂)_{g}+1(CH═CH₂), wherein each instance of g is, independently, 0 to 10, inclusive. Specific amino acids with terminal unsaturation are further described and depicted herein.

The term “amino acid analog” refers to a natural or unnatural amino acid where one or more of the C-terminal carboxy group, the N-terminal amino group and side-chain functional group has been chemically blocked, reversibly or irreversibly, or otherwise modified to another functional group. For example, aspartic acid-(beta-methyl ester) is an amino acid analog of aspartic acid; N-ethylglycine is an amino acid analog of glycine; or alanine carboxamide is an amino acid analog of alanine. Other amino acid analogs include methionine sulfoxide, methionine sulfone, S-(carboxymethyl)-cysteine, S-(carboxymethyl)-cysteine sulfoxide and S-(carboxymethyl)-cysteine sulfone.

### III. Administration of Therapeutic Compositions

Aspects of the present disclosure are directed to treatment or prevention of one or more diseases or conditions. In particular aspects, the present disclosure related to treatment or prevention of a disease or condition affected by expression of a gene under the control of a bZIP transcription factor (e.g., a bZIP transcription factor of Table 4 or FIGS. 21-98). For example, a condition of the disclosure may be a condition where overexpression of a gene under the control of a bZIP transcription factor contributes to the condition. Such conditions include, but are not limited to, cancer, fibrotic disorders, and diabetes. Therapeutic agents of the disclosure (e.g., bZIP transcriptional repressors) may be administered by one or more routes of administration. In some aspects, a bZIP transcriptional repressor is administered intravenously, intramuscularly, subcutaneously, topically, orally, transdermally, intraperitoneally, intraorbitally, by implantation, by inhalation, intrathecally, intraventricularly, or intranasally. In some aspects, a bZIP transcriptional repressor is administered intraveneously. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some aspects, a unit dose comprises a single administrable dose.

In some aspects, a therapeutic agent (e.g., bZIP transcriptional repressor) is administered at a dose of between 1 mg/kg and 5000 mg/kg. In some aspects, the therapeutic agent is administered at a dose of at least, at most, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110,111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, or 5000 mg/kg, or any range or value derivable therein.

The quantity to be administered, both according to number of treatments and unit dose, depends on the treatment effect desired. An effective dose is understood to refer to an amount necessary to achieve a particular effect. It is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

In certain instances, it will be desirable to have multiple administrations of the composition, e.g., 2, 3, 4, 5, 6 or more administrations. The administrations can be at 1, 2, 3, 4, 5, 6, 7, 8, to 5, 6, 7, 8, 9, 10, 11, or 12 week intervals, including all ranges there between.

The phrases “pharmaceutically acceptable” or “pharmacologically acceptable” refer to molecular entities and compositions that do not produce an adverse, allergic, or other untoward reaction when administered to an animal or human. As used herein, “pharmaceutically acceptable carrier” includes any and all solvents, dispersion media, coatings, anti-bacterial and anti-fungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. Supplementary active ingredients, such as other anti-infective agents and vaccines, can also be incorporated into the compositions.

The active compounds can be formulated for parenteral administration, e.g., formulated for injection via the intravenous, intramuscular, subcutaneous, or intraperitoneal routes. Typically, such compositions can be prepared as either liquid solutions or suspensions; solid forms suitable for use to prepare solutions or suspensions upon the addition of a liquid prior to injection can also be prepared; and, the preparations can also be emulsified.

The pharmaceutical forms suitable for injectable use include sterile aqueous solutions or dispersions; formulations including, for example, aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various anti-bacterial and anti-fungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization or an equivalent procedure. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Administration of the compositions will typically be via any common route. This includes, but is not limited to oral, or intravenous administration. Alternatively, administration may be by orthotopic, intradermal, subcutaneous, intramuscular, intraperitoneal, or intranasal administration. Such compositions would normally be administered as pharmaceutically acceptable compositions that include physiologically acceptable carriers, buffers or other excipients.

Upon formulation, solutions will be administered in a manner compatible with the dosage formulation and in such amount as is therapeutically or prophylactically effective. The formulations are easily administered in a variety of dosage forms, such as the type of injectable solutions described above.

### A. Cancer Therapy

In some aspects, the disclosed methods comprise administering a cancer therapy to a subject or patient. In some aspects, one or more of the DNA-binding dimers comprise the cancer therapy. In some aspects, the cancer therapy comprises the DNA-binding dimer and optionally another composition used to treat cancer. The cancer therapy may be chosen based on an expression level measurements, alone or in combination with the clinical risk score calculated for the subject. The cancer therapy may be chosen based on a genotype of a subject. The cancer therapy may be chosen based on the presence or absence of one or more polymorphisms in a subject. In some aspects, the cancer therapy comprises a local cancer therapy. In some aspects, the cancer therapy excludes a systemic cancer therapy. In some aspects, the cancer therapy excludes a local therapy. In some aspects, the cancer therapy comprises a local cancer therapy without the administration of a system cancer therapy. In some aspects, the cancer therapy comprises administration of a bZIP transcriptional repressor of the present disclosure. In some aspects, the cancer therapy comprises chemotherapy. In some aspects, the cancer therapy comprises radiotherapy. In some aspects, the cancer therapy comprises surgery. In some aspects, the cancer therapy comprises an immunotherapy, which may be a checkpoint inhibitor therapy. Any of these cancer therapies may also be excluded. Combinations of these therapies may also be administered.

The term “cancer,” as used herein, may be used to describe a solid tumor, metastatic cancer, or non-metastatic cancer. In certain aspects, the cancer may originate in the bladder, blood, bone, bone marrow, brain, breast, colon, esophagus, duodenum, small intestine, large intestine, colon, rectum, anus, gum, head, kidney, liver, lung, nasopharynx, neck, ovary, pancreas, prostate, skin, stomach, testis, tongue, or uterus. In some aspects, the cancer is a Stage I cancer. In some aspects, the cancer is a Stage II cancer. In some aspects, the cancer is a Stage III cancer. In some aspects, the cancer is a Stage IV cancer.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some aspects, the cancer is breast cancer. In some aspects, the cancer is triple negative breast cancer. For example, where the cancer is triple negative breast cancer, a therapeutic method may comprise administration of an engineered DNA-binding dimer capable of competing for DNA binding with a HIF protein (e.g., HIF1α and/or XBP1).

Methods may involve the determination, administration, or selection of an appropriate cancer “management regimen” and predicting the outcome of the same. As used herein the phrase “management regimen” refers to a management plan that specifies the type of examination, screening, diagnosis, surveillance, care, and treatment (such as dosage, schedule and/or duration of a treatment) provided to a subject in need thereof (e.g., a subject diagnosed with cancer).

### B. Fibrotic Disorder Therapy

In some aspects, the disclosed methods comprise administering a therapy for treating a fibrotic disorder. Fibrotic disorders contemplated herein include, but are not limited to, liver fibrosis, renal fibrosis, cardiac fibrosis, pulmonary fibrosis, idiopathic pulmonary fibrosis (IPF), scleroderma, psoriasis, and myelofibrosis. The present disclosure includes methods for treatment of a fibrotic disorder comprising administering to a subject an effective amount of a bZIP transcriptional repressor of the present disclosure. A bZIP transcriptional repressor of the disclosure may be used in combination with the administration of conventional therapies for fibrotic disorders, such as those known in the art.

### C. Diabetes Therapy

In some aspects, the disclosed methods comprise administering a therapy for treating diabetes. In some aspects, the diabetes is type 1 diabetes. In some aspects, the diabetes is type 2 diabetes. The present disclosure includes methods for treatment of diabetes comprising administering to a subject an effective amount of a bZIP transcriptional repressor of the present disclosure. A bZIP transcriptional repressor of the disclosure may be used in combination with the administration of conventional therapies, such as those known in the art and/or described below. For example, the current methods and compositions may be used in combination with traditional therapies for treating diabetes. Traditional therapies for diabetes include metformin, sulfonylureas, such as glyburide, glipizide, and glimepiride (Amaryl), meglitinides such as repaglinide and nateglinide, thiazolidinediones such as rosiglitazone and pioglitazone, DPP-4 inhibitors such as sitagliptin, saxagliptin, and linagliptin, GLP-1 receptor agonists such as exenatide and liraglutide, SGLT2 inhibitors such as canagliflozin and dapagliflozin, insulin therapy such insulin glulisine, insulin lispro, insulin aspart, insulin glargine, insulin detemir, and insulin isophane, and aspirin therapy.

### D. Pharmaceutical Compositions

In certain aspects, the compositions or agents for use in the methods, such as engineered DNA-binding dimers, are suitably contained in a pharmaceutically acceptable carrier. The carrier is non-toxic, biocompatible and is selected so as not to detrimentally affect the biological activity of the agent. The agents in some aspects of the disclosure may be formulated into preparations for local delivery or systemic delivery, in solid, semi-solid, gel, liquid or gaseous forms such as tablets, capsules, powders, granules, ointments, solutions, depositories, inhalants and injections allowing for oral, parenteral or surgical administration. Certain aspects of the disclosure also contemplate local administration of the compositions by coating medical devices and the like.

Suitable carriers for parenteral delivery via injectable, infusion or irrigation and topical delivery include distilled water, physiological phosphate-buffered saline, normal or lactated Ringer's solutions, dextrose solution, Hank's solution, or propanediol. In addition, sterile, fixed oils may be employed as a solvent or suspending medium. For this purpose any biocompatible oil may be employed including synthetic mono- or diglycerides. In addition, fatty acids such as oleic acid find use in the preparation of injectables. The carrier and agent may be compounded as a liquid, suspension, polymerizable or non-polymerizable gel, paste or salve.

The carrier may also comprise a delivery vehicle to sustain (i.e., extend, delay or regulate) the delivery of the agent(s) or to enhance the delivery, uptake, stability or pharmacokinetics of the therapeutic agent(s). Such a delivery vehicle may include, by way of non-limiting examples, microparticles, microspheres, nanospheres or nanoparticles composed of proteins, liposomes, carbohydrates, synthetic organic compounds, inorganic compounds, polymeric or copolymeric hydrogels and polymeric micelles.

In certain aspects, the actual dosage amount of a composition administered to a patient or subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

Solutions of pharmaceutical compositions can be prepared in water suitably mixed with a surfactant, such as hydroxypropylcellulose. Dispersions also can be prepared in glycerol, liquid polyethylene glycols, mixtures thereof and in oils. Under ordinary conditions of storage and use, these preparations contain a preservative to prevent the growth of microorganisms.

In certain aspects, the pharmaceutical compositions are advantageously administered in the form of injectable compositions either as liquid solutions or suspensions; solid forms suitable or solution in, or suspension in, liquid prior to injection may also be prepared. These preparations also may be emulsified. A typical composition for such purpose comprises a pharmaceutically acceptable carrier. For instance, the composition may contain 10 mg or less, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like.

Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, antifungal agents, anti-oxidants, chelating agents and inert gases. The pH and exact concentration of the various components the pharmaceutical composition are adjusted according to well-known parameters.

Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders.

In further aspects, the pharmaceutical compositions may include classic pharmaceutical preparations. Administration of pharmaceutical compositions according to certain aspects may be via any common route so long as the target tissue is available via that route. This may include oral, nasal, buccal, rectal, vaginal or topical. Alternatively, administration may be by orthotopic, intradermal, subcutaneous, intramuscular, intraperitoneal or intravenous injection. Such compositions would normally be administered as pharmaceutically acceptable compositions that include physiologically acceptable carriers, buffers or other excipients. For treatment of conditions of the lungs, aerosol delivery can be used. Volume of the aerosol may be between about 0.01 ml and 0.5 ml, for example.

An effective amount of the pharmaceutical composition is determined based on the intended goal. The term “unit dose” or “dosage” refers to physically discrete units suitable for use in a subject, each unit containing a predetermined-quantity of the pharmaceutical composition calculated to produce the desired responses discussed above in association with its administration, i.e., the appropriate route and treatment regimen. The quantity to be administered, both according to number of treatments and unit dose, depends on the protection or effect desired.

Precise amounts of the pharmaceutical composition also depend on the judgment of the practitioner and are peculiar to each individual. Factors affecting the dose include the physical and clinical state of the patient, the route of administration, the intended goal of treatment (e.g., alleviation of symptoms versus cure) and the potency, stability and toxicity of the particular therapeutic substance.

### Examples

The following examples are included to demonstrate certain aspects of the invention. It should be appreciated by those of skill in the art that the techniques disclosed in the examples which follow represent techniques discovered by the inventor to function well in the practice of the invention, and thus can be considered to constitute certain modes for its practice. However, those of skill in the art should, in light of the present disclosure, appreciate that many changes can be made in the specific aspects which are disclosed and still obtain a like or similar result without departing from the spirit and scope of the invention.

### Example 1—Design Pipeline for Generation of Synthetic DNA Binding Dimers

A DNA binding dimer is designed starting from natural bZIP proteins. First, amino acid sequences for first and second natural bZIP proteins are obtained from a sequence database (e.g., UniProt). For a heterodimer, the first and second proteins are different proteins. For a homodimer, the first and second proteins are the same protein. An example natural sequence for each of a first and second natural bZIP protein is provided below.

c-Fos (Homo sapiens):
(SEQ ID NO: 3)
MMFSGFNADYEASSSRCSSASPAGDSLSYYHSPADSFSSMGSPVNAQDF
CTDLAVSSANFIPTVTAISTSPDLQWLVQPALVSSVAPSQTRAPHPFGV
PAPSAGAYSRAGVVKTMTGGRAQSIGRRGKVEQLSPEEEEKRRIRRERN
KMAAAKCRNRRRELTDTLQAETDQLEDEKSALQTEIANLLKEKEKLEFI
LAAHRPACKIPDDLGFPEEMSVASLDLTGGLPEVATPESEEAFTLPLLN
DPEPKPSVEPVKSISSMELKTEPFDDFLFPASSRPSGSETARSVPDMDL
SGSFYAADWEPLHSGSLGMGPMATELEPLCTPVVTCTPSCTAYTSSFVF
TYPEADSFPSCAAAHRKGSSSNEPSSDSLSSPTLLAL
c-Jun (Homo sapiens):
(SEQ ID NO: 6)
MTAKMETTFYDDALNASFLPSESGPYGYSNPKILKQSMTLNLADPVGSL
KPHLRAKNSDLLTSPDVGLLKLASPELERLIIQSSNGHITTTPTPTFLC
PKNVTDEQEGFAEGFVRALAELHSQNTLPSVTSAAQPVNGAGMVAPAVA
SVAGGSGSGGFSASLHSEPPVYANLSNFNPGALSSGGGAPSYGAAGLAF
PAQPQQQQQPPHHLPQQMPVQHPRLQALKEEPQTVPEMPGETPPLSPID
MESQERIKAERKRMRNRIAASKCRKRKLERIARLEEKVKTLKAQNSELA
STANMLREQVAQLKQKVMNHVNSGCQLMLTQQLQTF

Second, the basic domain and leucine zipper domain of the first and second proteins are identified. The leucine zipper domain is identified based on the repetitive leucines every seven residues, plus three more residues toward the N-terminus from the first leucine. The basic domain is identified as the 26 residues immediately N-terminal of the first residue of the leucine zipper domain. Example sequences comprising the basic domain and leucine zipper domain are shown below.

c-Fos (Homo sapiens) basic domain and leucine
zipper domain:
(SEQ ID NO: 104)
MKRRIRRERNKMAAAKCRNRRRELTDTLQAETDQLEDEKSALQTEIANL
LKEKEKLEFILAAH
c-Jun (Homo sapiens) basic domain and leucine
zipper domain:
(SEQ ID NO: 105)
ERIKAEKRKMRNRIAASKCRKRKLERIARLEEKVKTLKAQNSELASTAN
MLREQVAQLKQKVMN

Third, the minimum necessary DNA recognition sequence of the first and second proteins is determined. The minimum necessary DNA recognition sequence is identified as the first 21 residues at the C-terminal end of the basic domain and the first 12 residues of the leucine zipper domain. Example minimum necessary DNA recognition sequences are provided below.

c-Fos (Homo sapiens) minimum necessary DNA
recognition sequence:
(SEQ ID NO: 106)
IRRERNKMAAAKCRNRRRELTDTLQAETDQLE
c-Jun (Homo sapiens) minimum necessary DNA
recognition sequence:
(SEQ ID NO: 107)
IRRERNKMAAAKCRNRRRELTDTLQAETDQLE

Fourth, all cysteines in the minimum necessary DNA recognition sequences of the first and second proteins are mutated based on the following rules:

If a cysteine is in the basic domain, replace it with a serine
If a cysteine is at a b, c, or f position of the leucine zipper domain, replace it with an alanine
If a cysteine is at an a or d position of the leucine zipper domain, replace it with a leucine.

Fifth, the linker position on each protein is identified. The linker position on the first protein is identified as the last residue of the basic domain. The linker position on the second protein is identified as the first residue at an e position of the leucine zipper domain. The linker residue on the first protein is replaced with a cysteine and the linker residue on the second protein is replaced with a Lys(mmt).

Sixth, weaker interhelix contact residues on the first and second proteins are replaced with stronger residues based on the following rules: If a residue at an a or d position of a leucine zipper domain is neither a leucine nor an isoleucine, replace it with a leucine.

If a residue at a gi position of the first protein and a paired residue at an cᵢ₊₁ position of the second protein are not either KE, EK, RE, ER, or QQ, replace the positions so that they are KE or RE (where the first letter indicates the residue at the gi position of the first protein and the second letter indicates the ei+1 position of the second protein).

Seventh, intrapeptide stabilizing linkage positions are identified for the first and second proteins. Intrapeptide stabilizing linkage positions are identified as either:
1) the fourth residue and the eighth residue of the minimum necessary DNA recognition sequence; or
2) the 22^{nd} residue and the 26^{th} residue of the minimum necessary DNA recognition sequence.

### Example 2—Modular bZIP Peptide Design

A general design was determined for bZIP peptide-derived synthetic transcriptional repressors (STRs) which can be applied to any bZIP protein. Table 9 shows components of the general design strategy. Positions a, b, c, d, e, f, g, h, i, j, k, 1, m, n, etc., reference the positions of specific amino acids in the leucine zipper relative to the basic/leucine zipper junction, where the junction signifies the bond between basic domain and leucine zipper domain, and where a, b, c, etc., is the first, second, third, etc., position after the junction, respectively. Table 10 shows example bZIP STRs designed from various bZIP proteins.

TABLE 9
General bZIP-derived synthetic transcriptional repressor (zSTR) design and modular
application to any bZIP protein

| | Representative Symbol and | |
|---|---|---|
| Items | Sequence | Notes |
| natural generic bZIP sequence | XXXXXXXXXXXXXXXXX | Throughout Table 9, ″/″ |
| around basic/leucine zipper | XXXXXXXX/XXXLXXXXX | signifies the bond between basic |
| regions of protein | XLXXXXXXLXXX | domain (left of ″/″) and leucine |
| | | zipper domain (right of ″/″) and |
| | | does not represent any chemical |
| | | entity |
| leucine zipper rule labeling | XXXXXXXXXXXXXXXXX | Positions a, b, c, d, e, f, g, h, |
| | XXXXXXXX/abcdefghijklmno | i, j, k, l, m, n, etc., reference |
| | pqrstuv | the positions of specific amino |
| | | acids in the leucine zipper |
| | | relative to the basic/leucine |
| | | zipper junction, where the |
| | | junction signifies the bond |
| | | between basic domain and |
| | | leucine zipper domain, and |
| | | where a, b, c, etc., is the first, |
| | | second, third, etc., position after |
| | | the junction, respectively. |
| leucine zipper numbering | /XXXLXXXXXXLXXXXXX | The position for every repetitive |
| | LXXX | heptad will be abcdefg where d |
| | | will be Leucine |
| Peptide length within bZIP | XXXXXXXXXXXXXXXXX | The minimized required |
| protein necessary and | XXX/XXXXXXXXXXXX | sequence will be first 20 |
| sufficient for high affinity | | residues from the C-terminus of |
| zSTR design and engineering; | | basic domain and the first 12 |
| | | residues from the N-terminus of |
| | | Leucine zipper domain |
| possible extension of length | XXXXXXXXXXXXXXXXX | One possible extension of STR |
| of zSTR | XXXXXX/XXXXXXXXXXX | will be first 23 residues from the |
| | X | C-terminus of DNA binding |
| | | region |
| | XXXXXXXXXXXXXXXXX | One possible extension of STR |
| | XXXXXXXX/XXXXXXXXX | will be first 25 residues from the |
| | XXX | C-terminus of DNA binding |
| | | region |
| Generic sequence cyclizing | XXX^{∧}XXX^{∧}XXXXXXXXXX | ″^{∧}″ are cyclizing positions |
| positions | XX/XXXLXXXXXXLX | (residues used for peptide |
| | | stapling) |
| | XXXXXXXXXXXXXXXXX | ″^{∧}″ are cyclizing positions |
| | XXX/X^{∧}XLX^{∧}XXXXLX | (residues used for peptide |
| | | stapling) |
| Additional generic sequence | XXXXXXX^{∧}XXX^{∧}XXXXXX | ″^{∧}″ are cyclizing positions |
| cyclizing positions | XX/XXXLXXXXXXLX | (residues used for peptide |
| | | stapling) |
| | XXX^{∧}XXXXXXX^{∧}XXXXXX | ″^{∧}″ are cyclizing positions |
| | XX/XXXLXXXXXXLX | (residues used for peptide |
| | | stapling) |
| | XXXXXXX^{∧}XXX^{∧}XXX^{∧}XXX | ″^{∧}″ are cyclizing positions |
| | XX/XXXLXXXXXXLX | (residues used for peptide |
| | | stapling) |
| | XXXXXXXXXXXXXX^{∧}XX | ″^{∧}″ are cyclizing positions |
| | XX/XXXLXXXXXXLX | (residues used for peptide |
| | | stapling) |
| | XXXXXXXXXXXXXXXXX | ″^{∧}″ are cyclizing positions |
| | X^{∧}X/XXXLX^{∧}XXXXLX | (residues used for peptide |
| | | stapling) |
| | XXXXXXXXXXXXXXXXX | ″^{∧}″ are cyclizing positions |
| | X^{∧}X/XX^{∧}LXXXXXXLX | (residues used for peptide |
| | | stapling) |
| | XXXXXXXXXXXXXXXXX | ″^{∧}″ are cyclizing positions |
| | XXX/XXXLX^{∧}XXX^{∧}LX | (residues used for peptide |
| | | stapling) |
| | XXXXXXXXXXXXXXXXX | ″^{∧}″ are cyclizing positions |
| | XXX/XX^{∧}LXXXXX^{∧}LX | (residues used for peptide |
| | | stapling) |
| Generic sequence sites for | monomer1-DimerA | XXXXXXXXXXXXXXXXXX |
| interpeptide linkage positions | (first engineered peptide) | X#/XXXLXXXXXXLX |
| (denoted by ′#′ symbol) | monomer2-DimerA | XXXXXXXXXXXXXXXXXX |
| | (second engineered peptide) | XX/XXXL#XXXXXLX |
| | monomer1-DimerB | XXXXXXXXXXXXXXXXXX |
| | (first engineered peptide) | XX/XXXL#XXXXXLX |
| | monomer2-DimerB | XXXXXXXXXXXXXXXXXX |
| | (second engineered peptide) | X#/XXXLXXXXXXLX |
| Additional generic sequence | monomer1-DimerC | XXXXXXXXXXXXXXXXXX |
| sites for dimerizing mutations | (first engineered peptide) | XX/XXXLXX#XXXLX |
| | monomer2-DimerC | XXXXXXXXXXXXXXXXXX |
| | (second engineered peptide) | XX/XXXLXXXXXXL# |
| | monomer1-DimerD | XXXXXXXXXXXXXXXXXX |
| | (first engineered peptide) | XX/XXXLXXXXXXL# |
| | monomer2-DimerD | XXXXXXXXXXXXXXXXXX |
| | (second engineered peptide) | XX/XXXL#XXXXXLX |
| mandatory changes on the | monomer1 | XXXXXXXXXXXXXXXXXX |
| residues of the leucine zipper | (first engineered peptide) | XX/*XXLXXX*XXLX |
| at ′a′ position | monomer2 | XXXXXXXXXXXXXXXXXX |
| (if residues at * from natural | (second engineered peptide) | XX/*XXLXXX*XXLX |
| protein sequence are not Leu, | | |
| Ile, need to change into the | | |
| combination into: LL, LI or II) | | |
| mandatory changes on the | monomer1 | XXXXXXXXXXXXXXXXXX |
| residues of the leucine zipper | (first engineered peptide) | X&/XXXLXXXXXXLX |
| at g(i)e′(i+1) position | monomer2 | XXXXXXXXXXXXXXXXXX |
| (If residues at ′&′ postion | (second engineered peptide) | XX/XXXL&XXXXXLX |
| from natural protein sequence | | |
| are not QQ, KE or RE, QE | | |
| combination, need to change | | |
| resiude combination to QQ, | | |
| KE, RE or QE) | | |
| mandatory changes on the | monomer1 | XXXXXXXXXXXXXXXXXX |
| residues of the leucine zipper | (first engineered peptide) | XX/XXXLXX&XXXLX |
| at g(i)e′(i+1) position | monomer2 | XXXXXXXXXXXXXXXXXX |
| (If residues at ′&′ postion | (second engineered peptide) | XX/XXXLXXXXXXL& |
| from natural protein sequence | | |
| are not QQ, KE or RE, QE | | |
| combination, need to change | | |
| resiude combination to QQ, | | |
| KE, RE or QE) | | |
| Example- | monomer1 | RRK^{∧}KNR^{∧}AAQTARDRKKA |
| STR22 | (first engineered peptide) | #LSELEQQVVDLE |
| from XBP1 | monomer2 | RRK^{∧}KNR^{∧}AAQTARDRKKA |
| | (second engineered peptide) | RLSEL#QQVVDLE |
| Example- | monomer1 | IRR^{∧}RNK^{∧}AAAKSRNRRRRE |
| FJ191 (also “FJSTR91”) from | (first engineered peptide) | #IDEIQAEIEQIE |
| Fos and Jun | monomer2 | RRK^{∧}RNR^{∧}AASKSRKRKLER |
| | (second engineered peptide) | IARL#EKIKTLK |

TABLE 10
Engineered peptides from various bZIP proteins

| protein name | abbreviation | engineered peptide sequence |
|---|---|---|
| CREB/ATF bZIP | CREBZF | SPRKAAAAAARLNRLKKKEYVMGLESRV |
| transcription factor | | RGLA |
| Endoplasmic reticulum | NFE2L1 | RRRGKNKMAAQNSRKRKLDTILNLERDV |
| membrane sensor | | EDLQ |
| NFE2L1 | | |
| Basic leucine zipper | BATF | QRREKNRIAAQKCRQRQTQKADTLHLESE |
| transcriptional factor | | DLE |
| ATF-like | | |
| Transcription regulator | BACH2 | RRRSKNRIAAQRCRKRKLDCIQNLESEIRK |
| protein BACH2 | | LV |
| Transcription regulator | BACH1 | RRRSKNRIAAQRCRKRKLDCIQNLESEIEK |
| protein BACH1 | | LQ |
| Neural retina-specific | NRL | RRTLKNRGYAQACRSKRLQQRRGLEAER |
| leucine zipper protein | | ARLA |
| Cyclic AMP-responsive | CREB3L4 | RRKKEYIDGLESRVAACSAQNQELQKKV |
| element-binding protein | | QELE |
| 3-like protein 4 | | |
| Transcription factor | MafG | RRTLKNRGYAASCRVKRVTQKEELEKQK |
| MafG | | AELQ |
| Nuclear factor erythroid | NRF2 | RRRGKNKVAAQNCRKRKLENIVELEQDL |
| 2-related factor 2 | | DHLK |
| Nuclear factor | NFIL3 | EKRRKNNEAAKRSREKRRLNDLVLENKLI |
| interleukin-3-regulated | | ALG |
| protein | | |
| Transcription factor NF- | NFE2 | RRRGKNKVAAQNCRKRKLETIVQLERELE |
| E2 45 kDa subunit | | RLT |
| Transcription factor | MafK | RRTLKNRGYAASCRIKRVTQKEELERQRV |
| MafK | | ELQ |
| Cyclic AMP-responsive | CREB 1 | VRLMKNREAARESRRKKKEYVKSLENRV |
| element-binding protein 1 | | AVLE |
| Cyclic AMP-responsive | CREB3L2 | RKKKEYMDSLEKKVESCSTENLELRKKVE |
| element-binding protein | | VLE |
| 3-like protein 2 | | |
| Hepatic leukemia factor | HLF | MAAKRSRDARRLKENQIAIRASFLEKENS |
| | | ALR |
| Proto-oncogene c-Fos | cFOS | IRRERNKMAAAKCRNRRRELTDTLQAETD |
| | | QLE |
| Transcription factor Maf | Maf | RRTLKNRGYAQSCRFKRVQQRHVLESEK |
| | | NQLL |
| Transcription factor MafF | MafF | RRTLKNRGYAASCRVKRVSQKEELQKQK |
| | | SELE |
| Transcription factor AP-1 | cJUN | RKRMRNRIAASKSRKRKLERIARLEEKVK |
| | | TLK |
| Transcription factor | MafA | RRTLKNRGYAQSSRFKRVQQRHILESEKC |
| MafA | | QLQ |
| Cyclic AMP-dependent | ATF6a | KKKKEYMLGLEARLKAALSENEQLKKEN |
| transcription factor ATF- | | GTLK |
| 6 alpha | | |
| CAMP-responsive | CREM | LRLMKNREAAKESRRRKKEYVKSLESRV |
| element modulator | | AVLE |
| Cyclic AMP-responsive | CREB3 | RKKKVYVGGLESRVLKYTAQNMELQNK |
| element-binding protein 3 | | VQLL |
| Cyclic AMP-dependent | ATF1 | IRLMKNREAARECRRKKKEYVKSLENRV |
| transcription factor ATF- | | AVLE |
| 1 | | |
| Basic leucine zipper | ATFL3 | RRREKNRVAAQRSRKKQTQKADKLHEEY |
| transcriptional factor | | ESLE |
| ATF-like 3 | | |
| Cyclic AMP-dependent | ATF7 | RFLERNRAAASRSRQKRKLWVSSLEKKAE |
| transcription factor ATF- | | ELT |
| 7 | | |
| Cyclic AMP-dependent | ATF6b | RQQRMIKNRESASQSRRKKKEYLQGLEAR |
| transcription factor ATF- | | LQ |
| 6 beta | | |
| Cyclic AMP-responsive | CREB3L1 | RKKKEYVESLEKKVETFTSENNELWKKVE |
| element-binding protein | | TLE |
| 3-like protein 1 | | |
| Cyclic AMP-responsive | CREB5 | KFLERNRAAATRSRQKRKVWVMSLEKKA |
| element-binding protein 5 | | EELT |
| Basic leucine zipper | ATFL2 | LKKQKNRAAAQRSRQKHTDKADALHQQ |
| transcriptional factor | | HESLE |
| ATF-like 2 | | |
| Cyclic AMP-responsive | CREB3L3 | KKKKEYIDGLETRMSASTAQNQELQRKVL |
| element-binding protein | | HLE |
| 3-like protein 3 | | |
| Cyclic AMP-dependent | ATF4 | KKMEQNKTAATRYRQKKRAEQEALTGES |
| transcription factor ATF- | | KELE |
| 4 | | |
| Cyclic AMP-dependent | ATF3 | RRRERNKIAAAKSRNKKKEKTESLQKESE |
| transcription factor ATF- | | KLE |
| 3 | | |
| Fos-related antigen 1 | FRA1 | RRERNKLAAAKSRNRRKELTDFLQAETDK |
| | | LE |
| Protein fosB | FOSB | VRRERNKLAAAKSRNRRRELTDRLQAET |
| | | DQLE |
| Cyclic AMP-dependent | ATF2 | KFLERNRAAASRSRQKRKVWVQSLEKKA |
| transcription factor ATF- | | EDLS |
| 2 | | |
| D site-binding protein | DBP | SRRYKNNEAAKRSRDARRLKENQISVRAA |
| | | FLE |
| Jun dimerization protein 2 | JDP2 | RRREKNKVAAARSRNKKKERTEFLQRESE |
| | | RLE |
| Transcription factor jun-B | JUNB | RKRLRNRLAATKSRKRKLERIARLEDKVK |
| | | TLK |
| Transcription factor jun-D | JUND | RKRLRNRIAASKSRKRKLERISRLEKVKTL |
| | | K |
| Fos-related antigen 2 | FRA2 | IGTTVGRRRRDEQLSPEEEEKRRIRRERNK |
| | | LA |
| Transcription factor MafB | MAFB | RRTLKNRGYAQSSRYKRVQQKHHLENEK |
| | | TQLI |
| X-box-binding protein 1 | XBP1 | RRKLKNRVAAQTARDRKKARMSELEQQV |
| | | VDLE |
| Cyclic AMP-dependent | ATF5 | KKRDQNKSAALRYRQRKRAEGEALEGES |
| transcription factor ATF- | | QGLE |
| 5 | | |
| CCAAT/enhancer- | CEBPd | IAVRKSRDKAKRRNQEMQQKLVELSAEN |
| binding protein delta | | EKLH |
| CCAAT/enhancer- | CEBPy | MAVKKSRLKSKQKAQDTLQRVNQLKEEN |
| binding protein gamma | | ERLE |
| CCAAT/enhancer- | CEBPb | YKIRRERNNIAVRKSRDKAKMRNLETQHK |
| binding protein beta | | VLE |
| CCAAT/enhancer- | CEBPa | IAVRKSRDKAKQRNVETQQKVLELTSDND |
| binding protein alpha | | RLR |
| CCAAT/enhancer- | CEBPe | YRLRRERNNIAVRKSRDKAKRRILETQQK |
| binding protein epsilon | | VLE |
| CREB3 regulatory factor | CREBRF | SDLTPVSELPLTARPRSRKEKNKLASRASR |
| | | LK |
| DNA damage-inducible | DDIT3 | SPARAGKQRMKEKEQENERKVAQLAEEN |
| transcript 3 protein | | ERLK |
| Thyrotroph embryonic | TEF | DEKYWTRRKKNNVAAKRSRDARRLKEN |
| factor | | QITIR |
| CAMP-responsive | CEBPL2 | ESRARKKLRYQYLEELVSSRERAISALREE |
| element-binding protein- | | LE |
| like 2 | | |
| Nuclear factor erythroid | NRF3 | RRRGKNKVAAQNSRKRKLDIILNLEDDVS |
| 2-related factor 3 | | NLQ |
| NFIL3 like protein | NFILZ | EKRRKNNEAAKRSREKRRLNDAAIEGRLA |
| | | ALM |

### Example 3—Example Synthetic DNA Binding Dimers

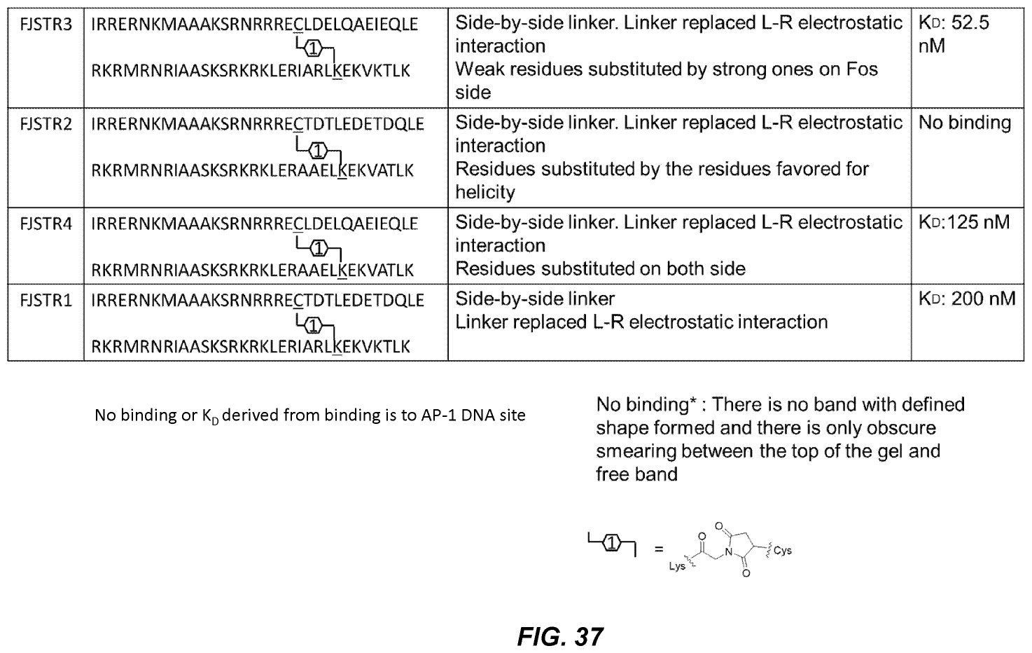

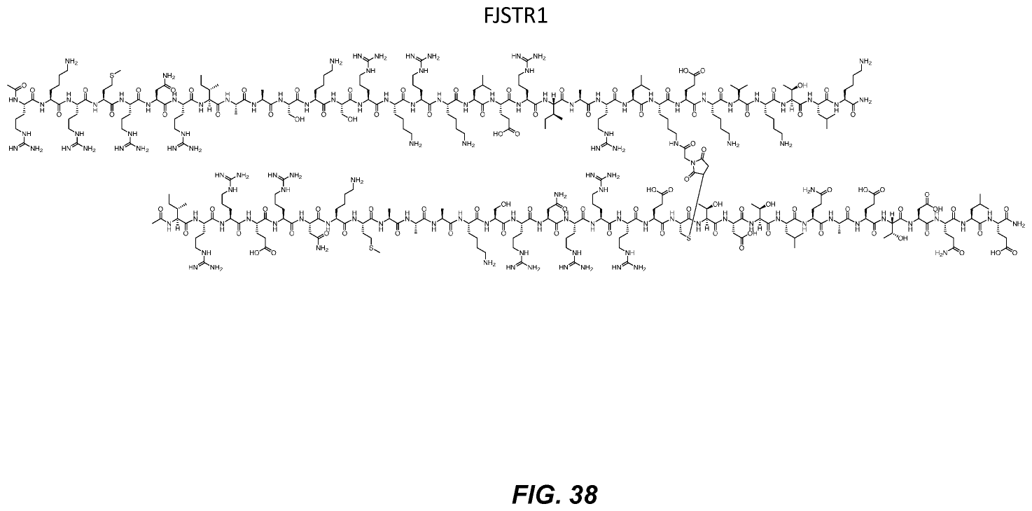

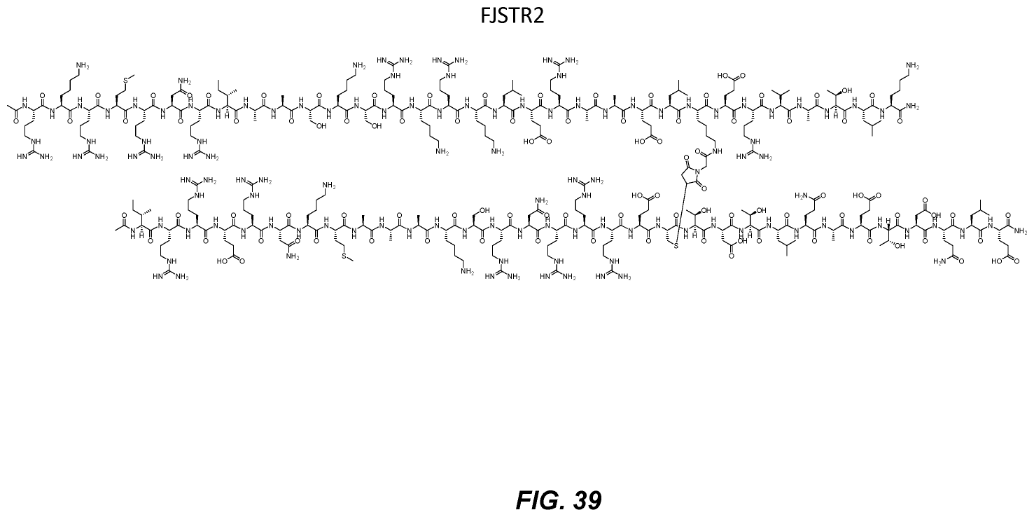

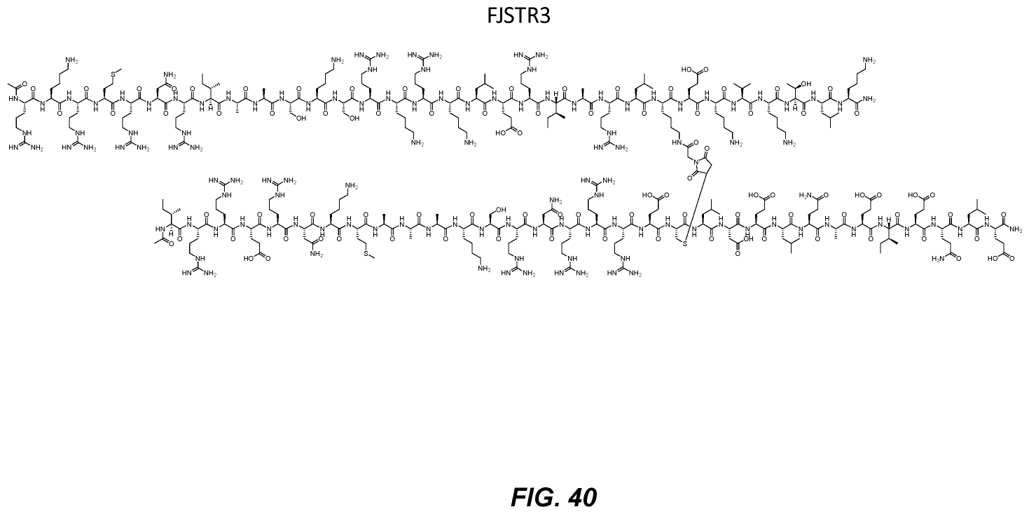

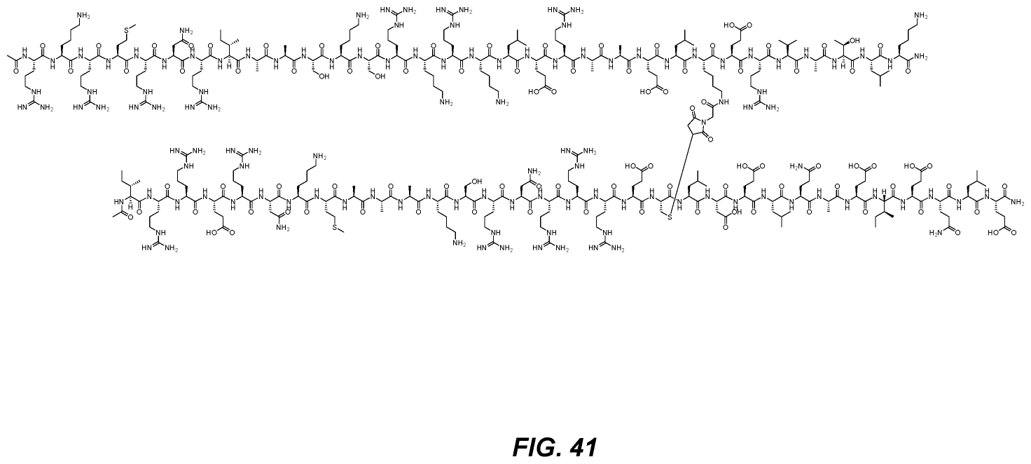

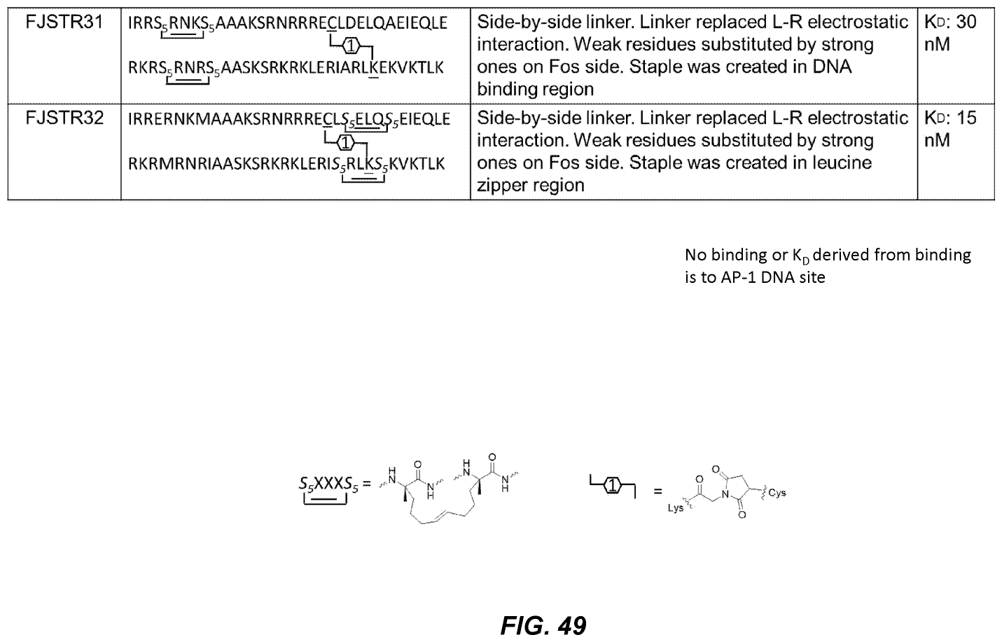

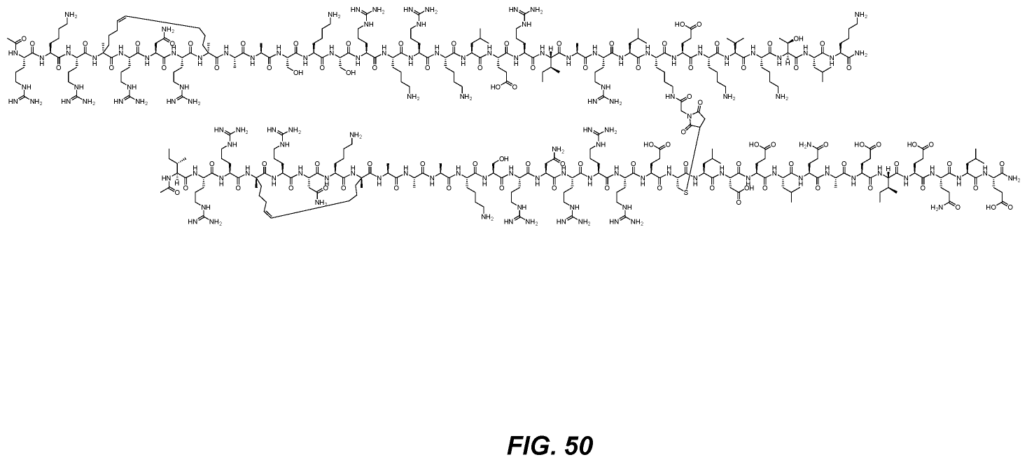

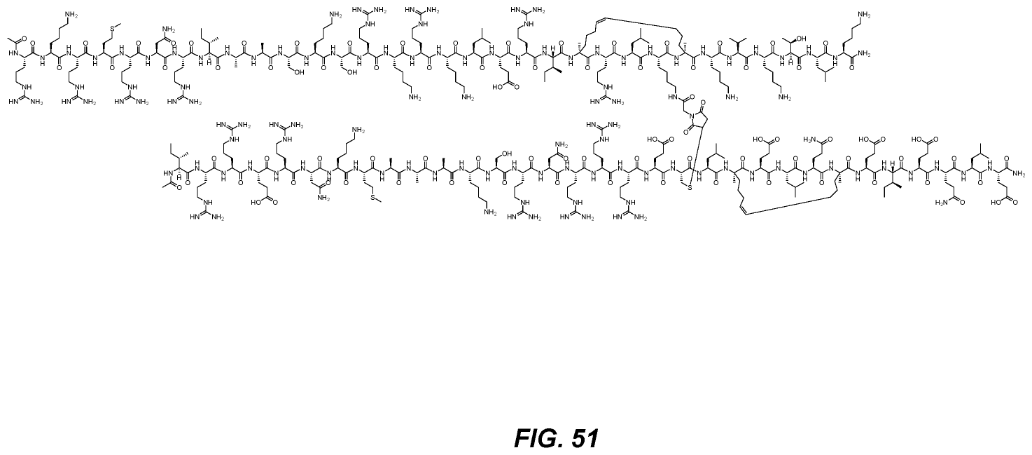

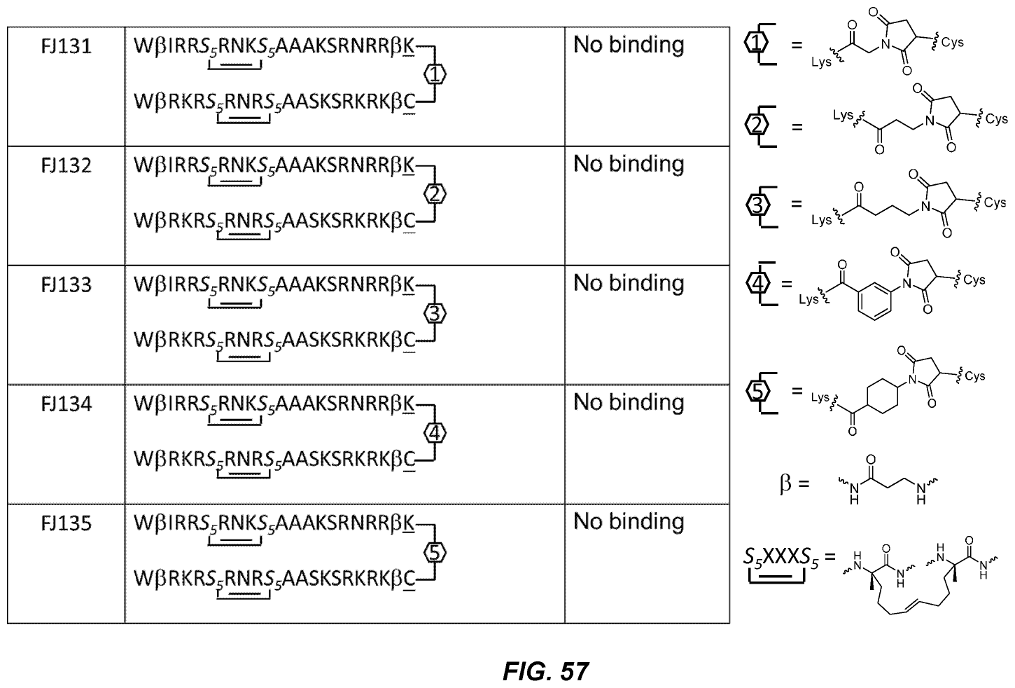

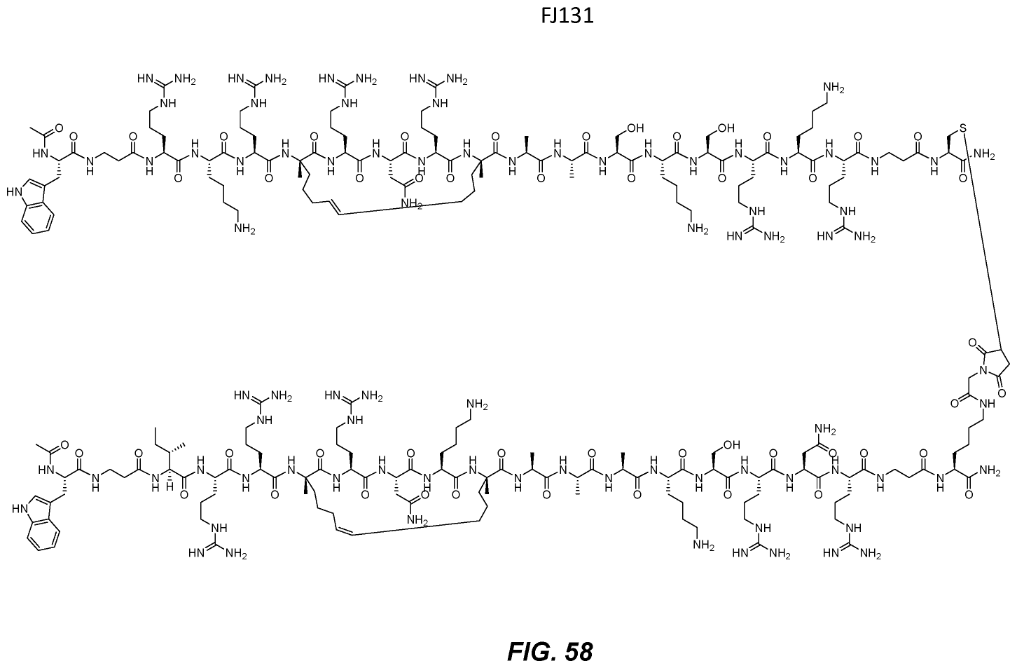

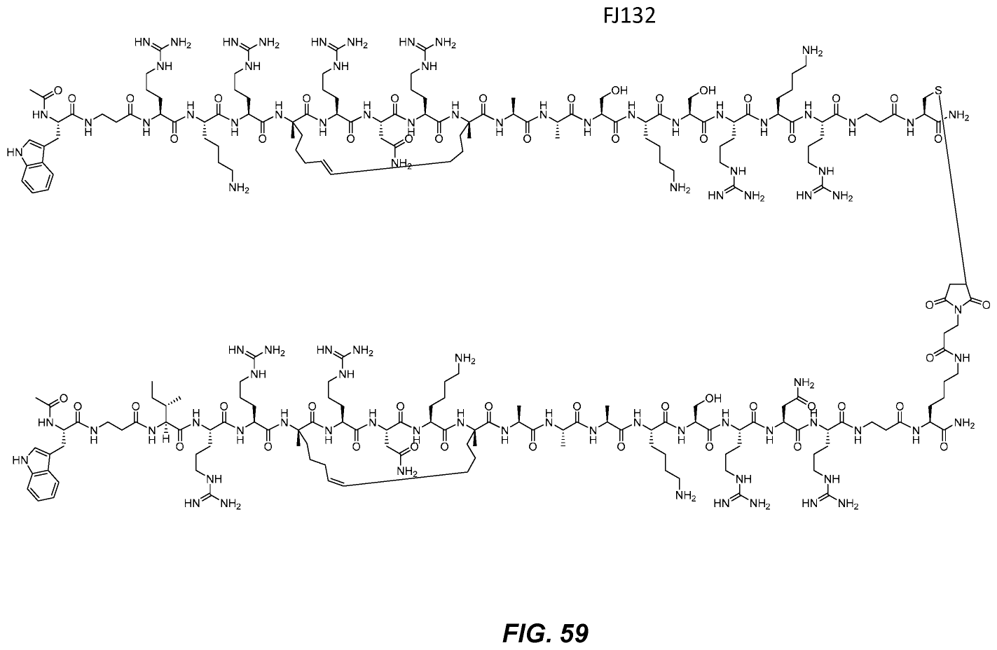

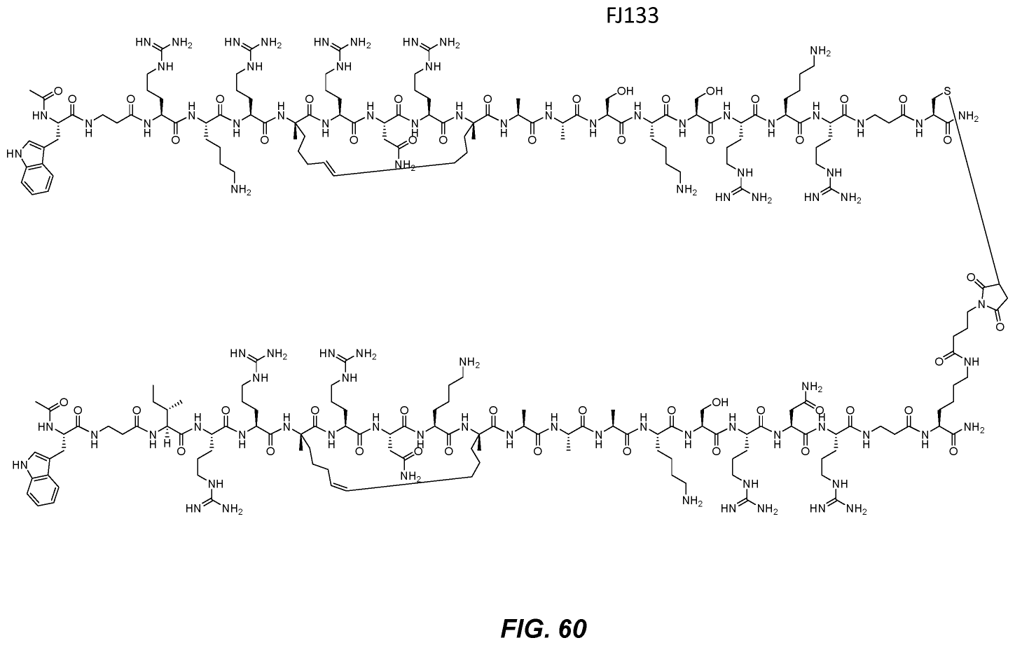

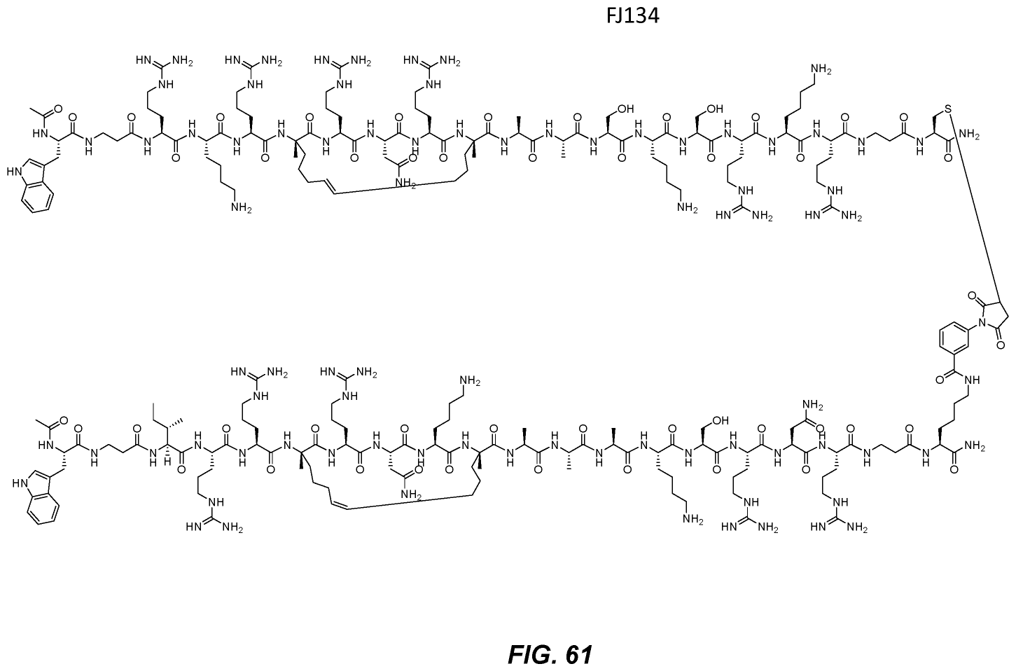

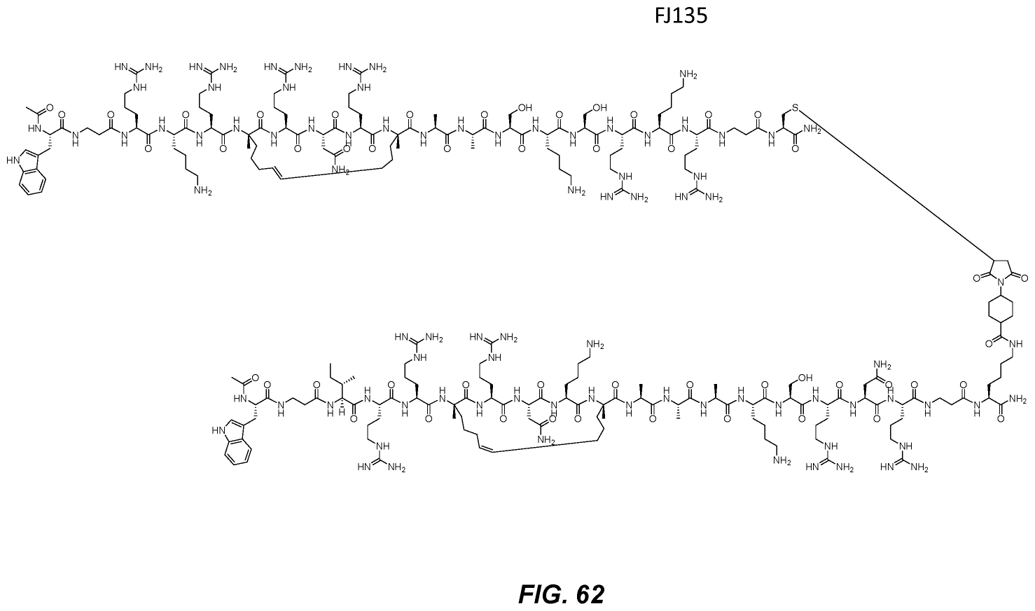

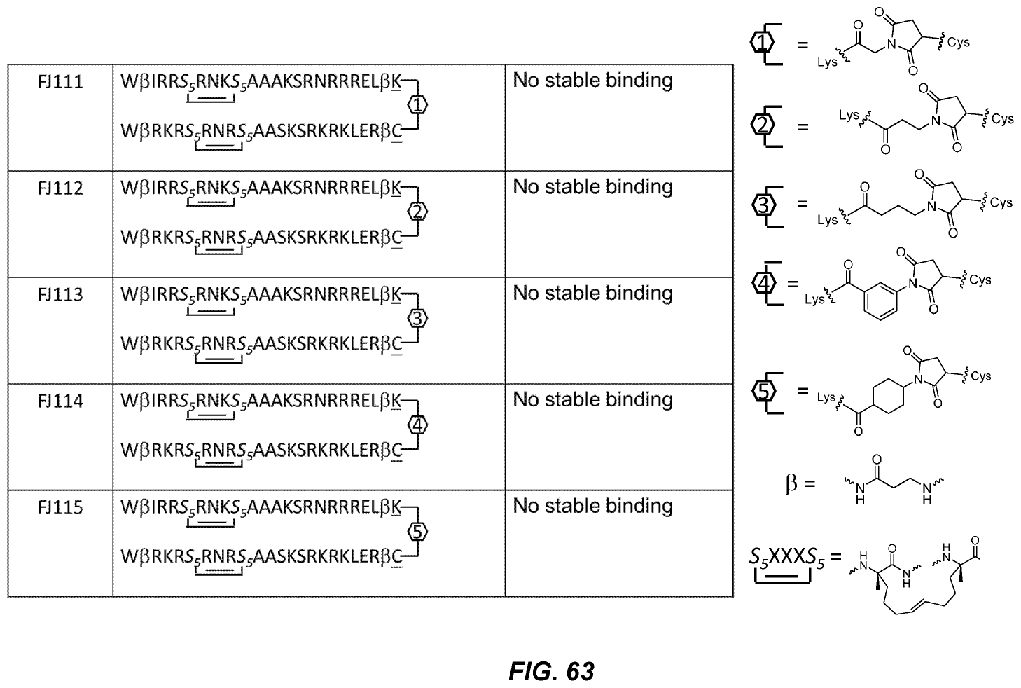

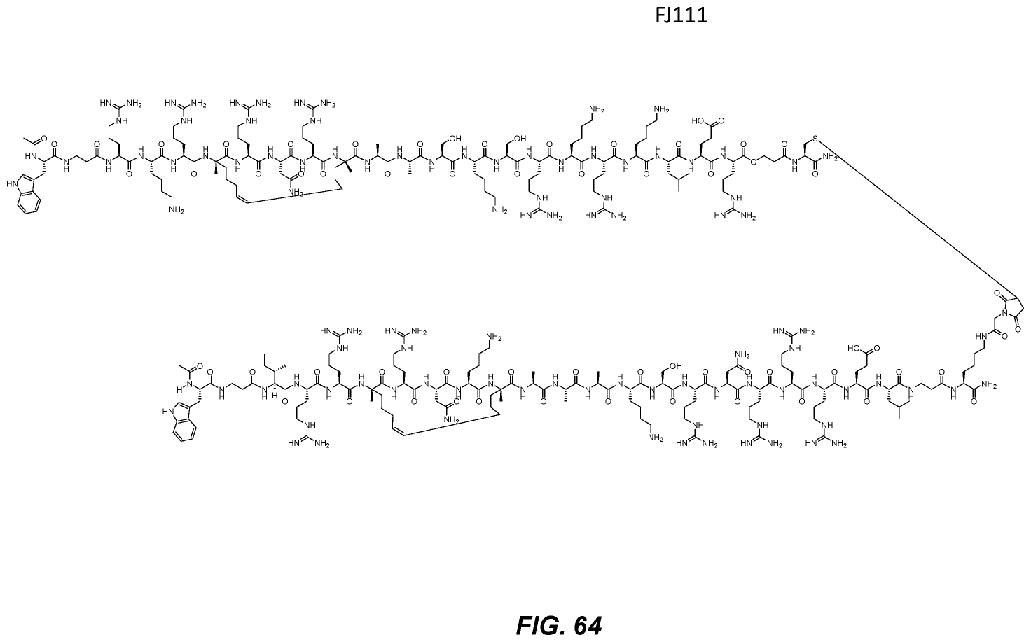

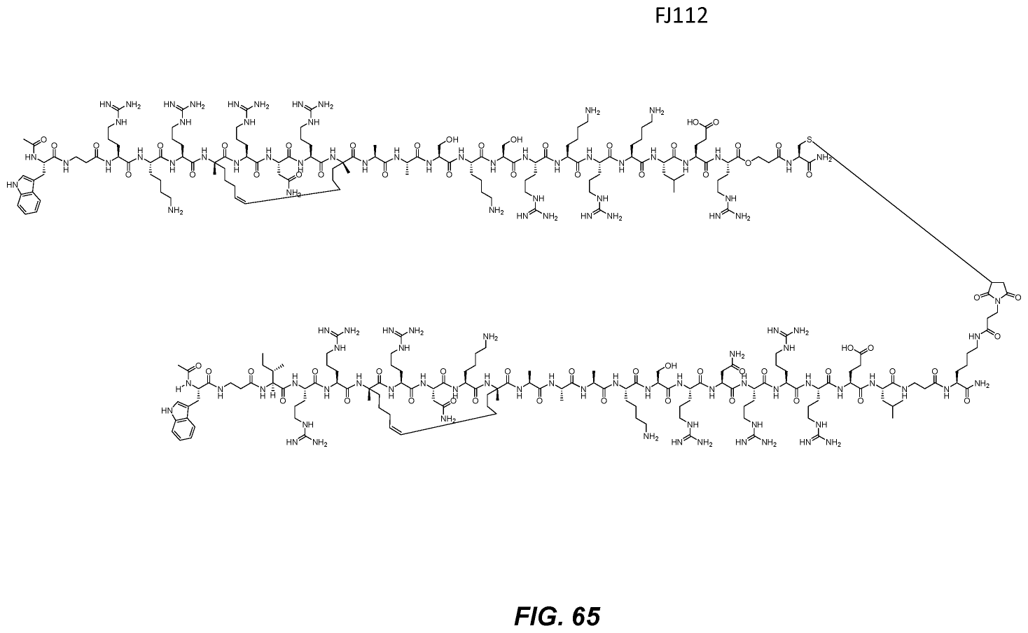

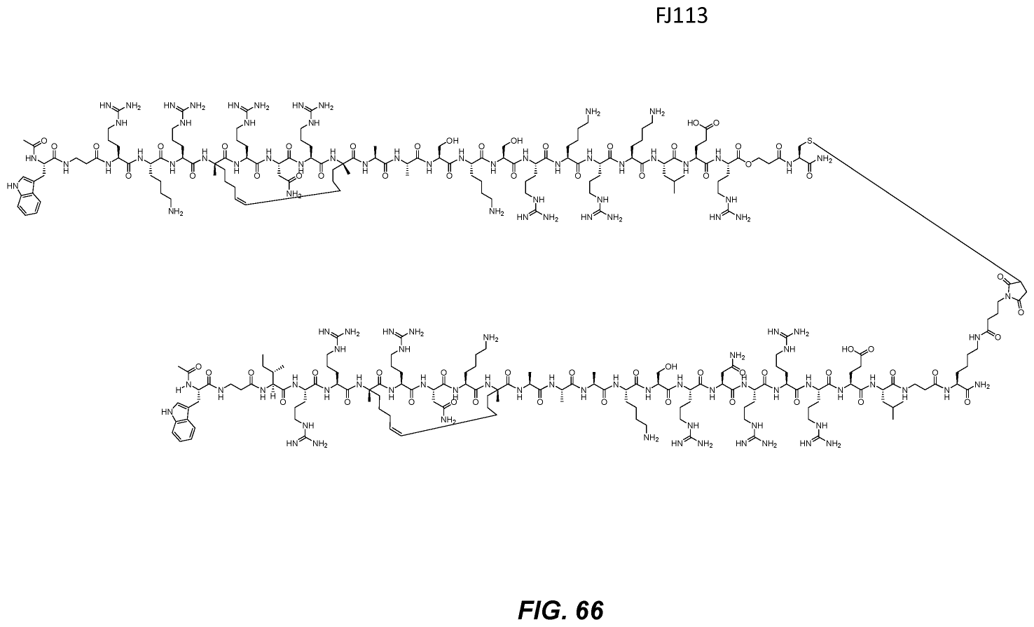

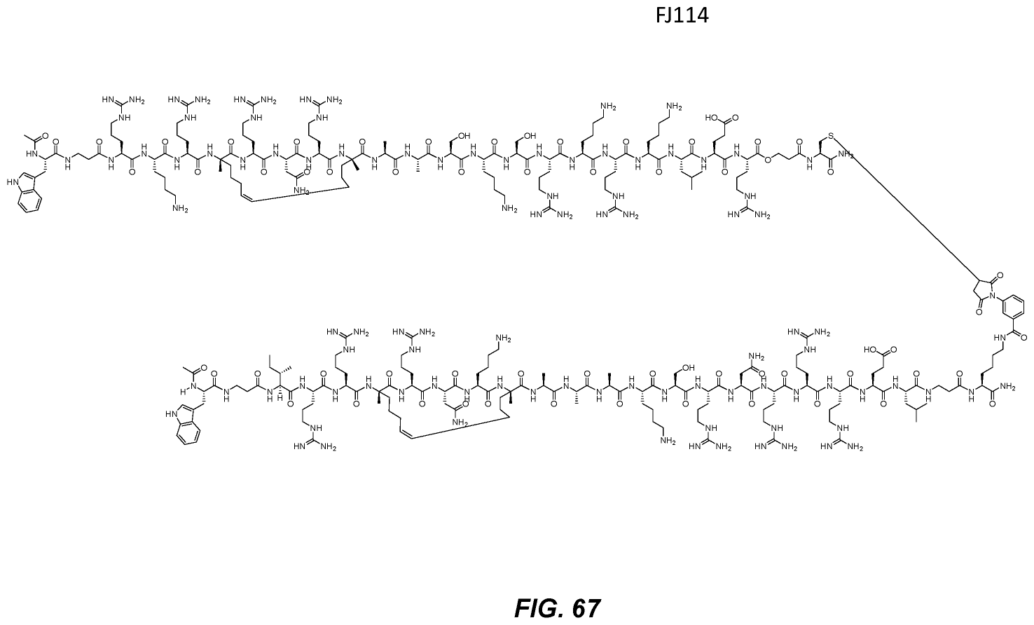

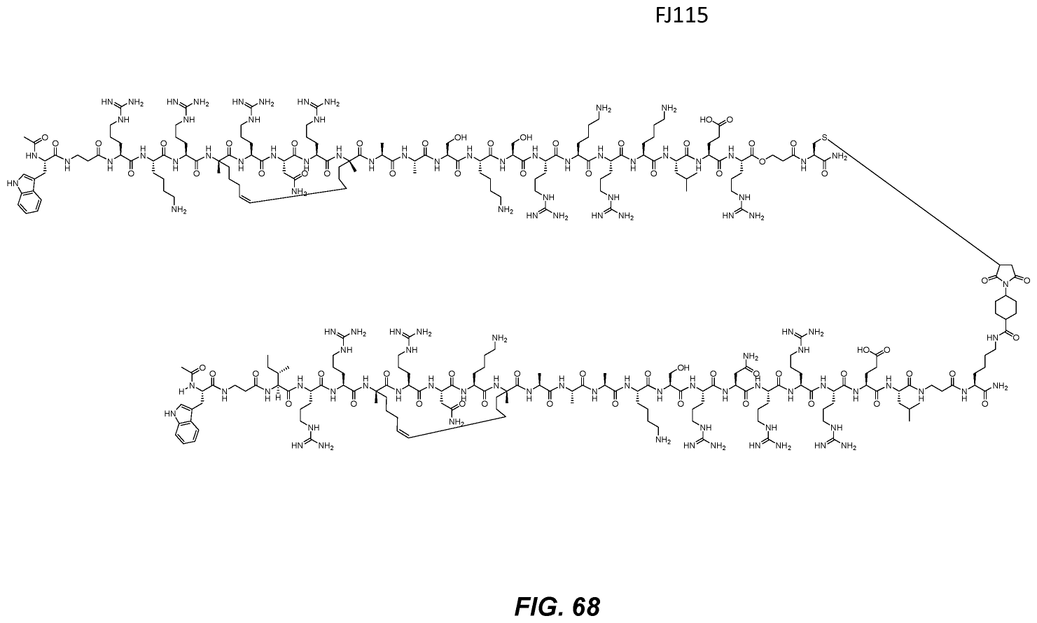

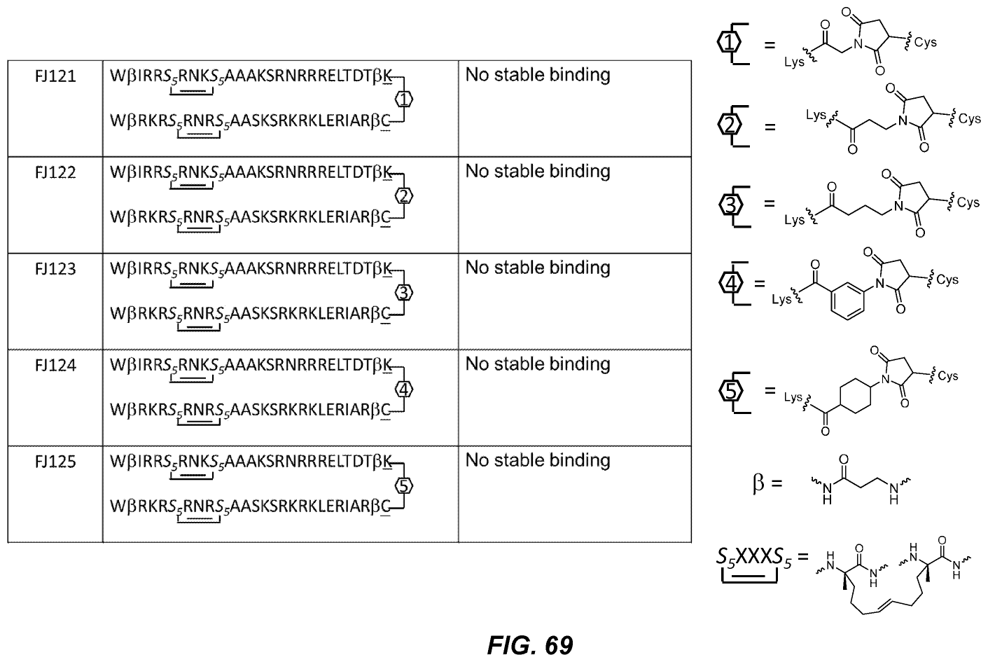

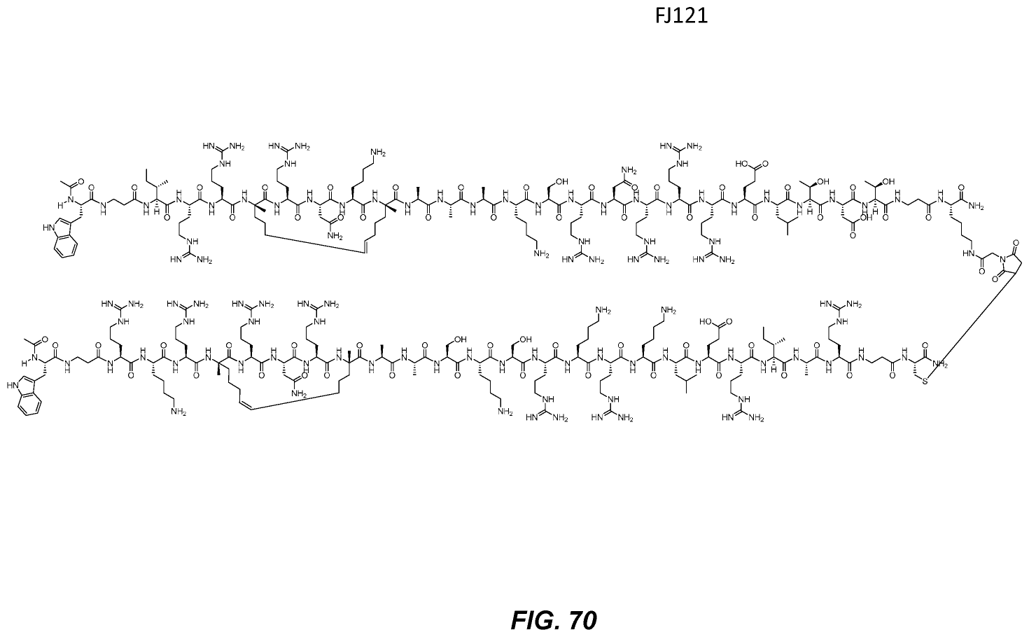

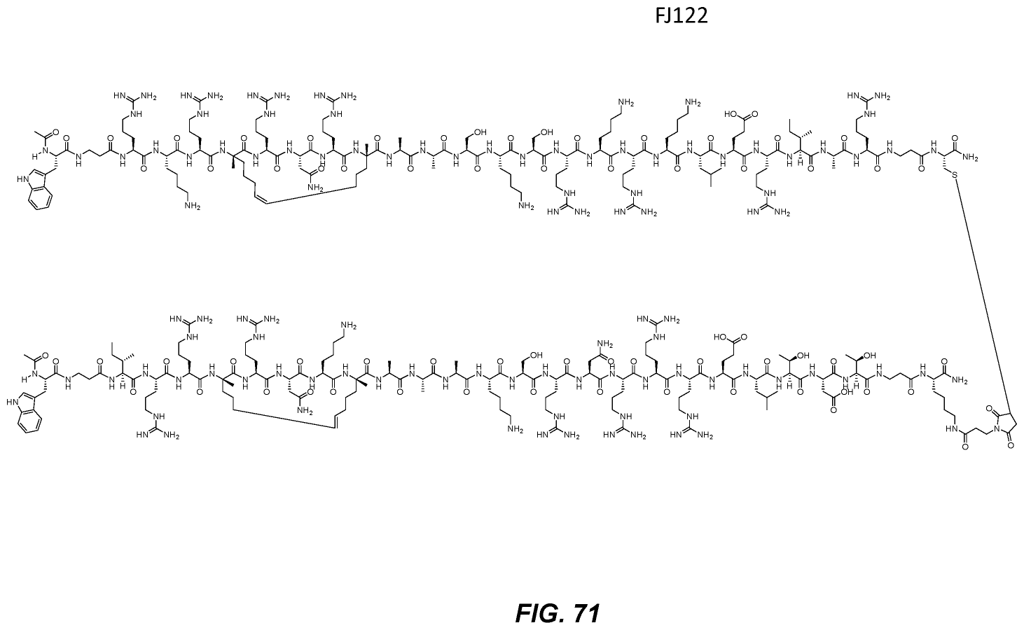

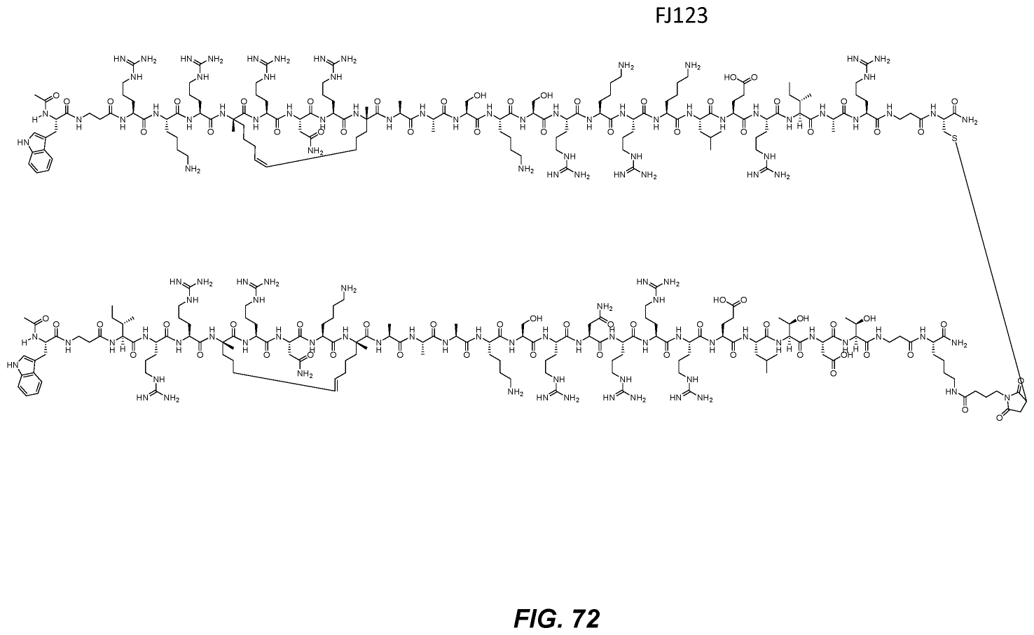

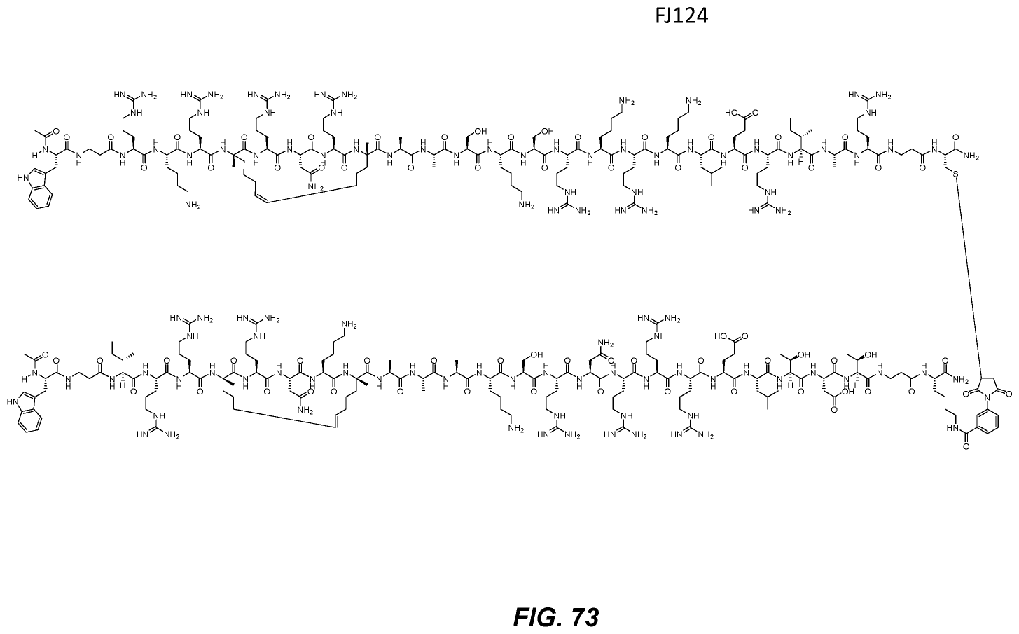

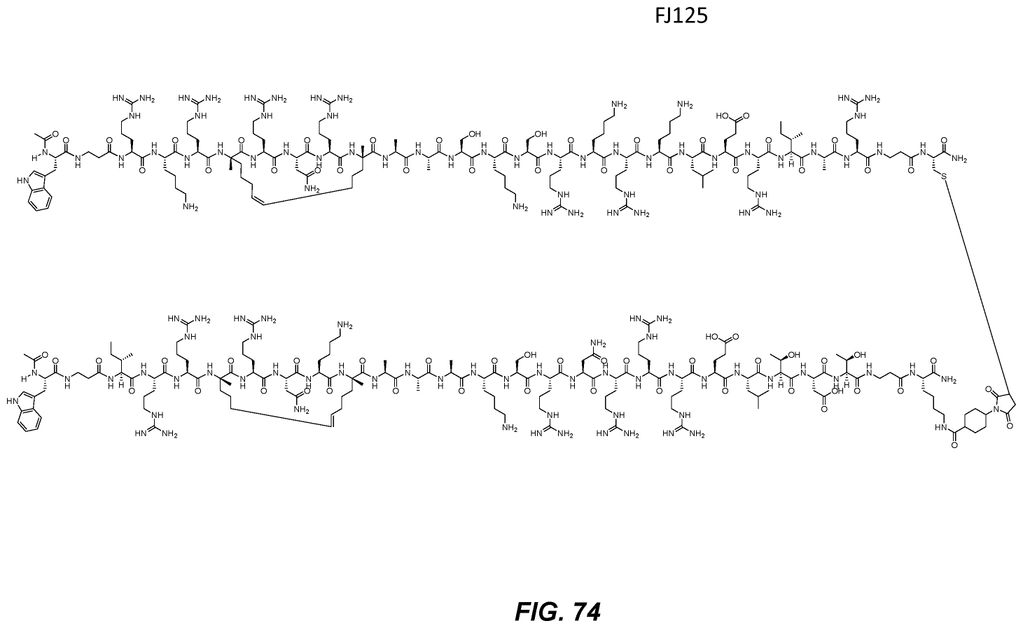

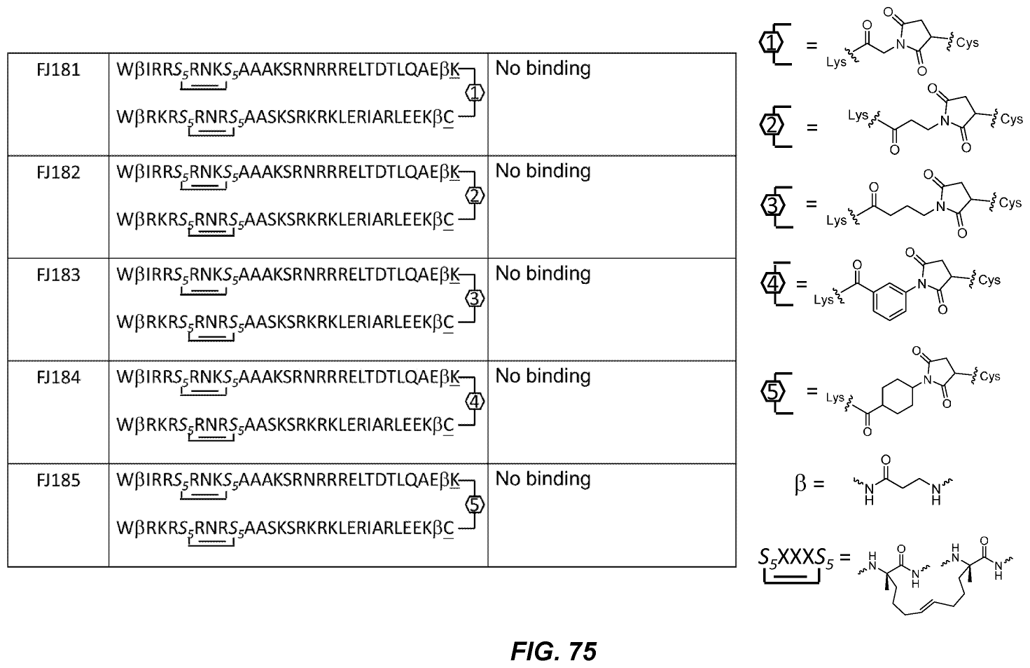

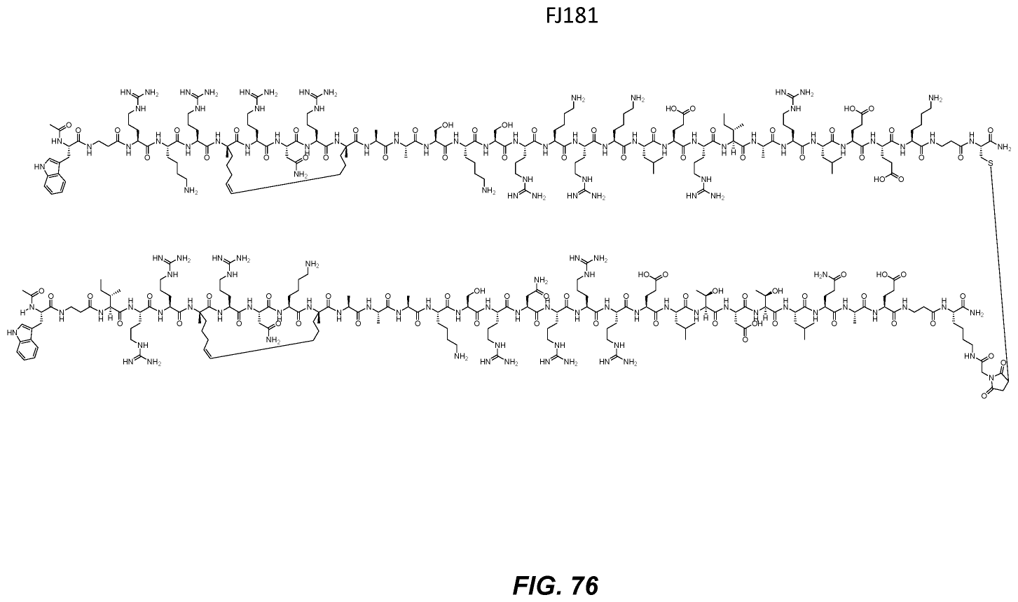

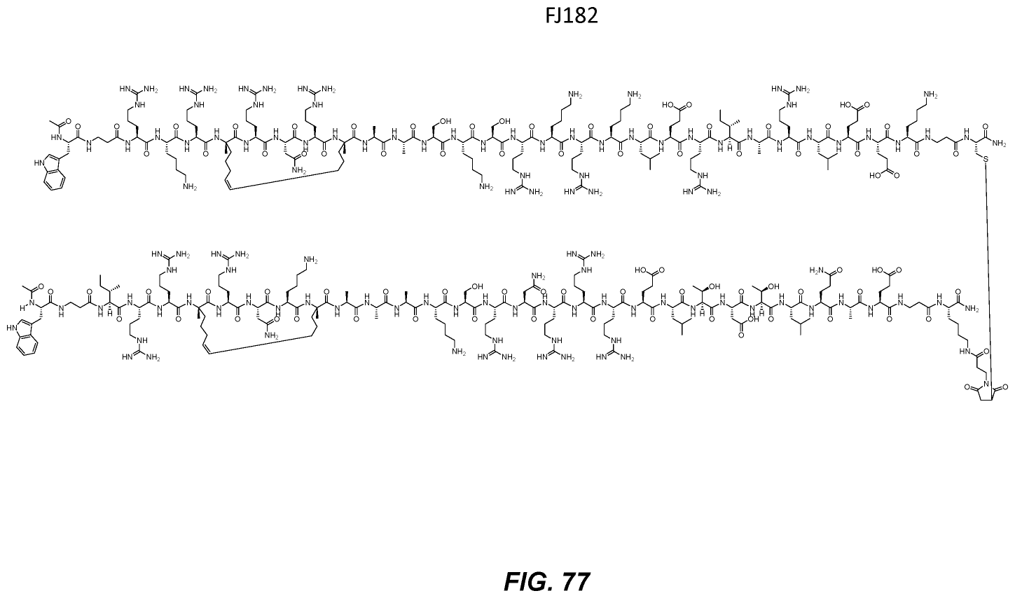

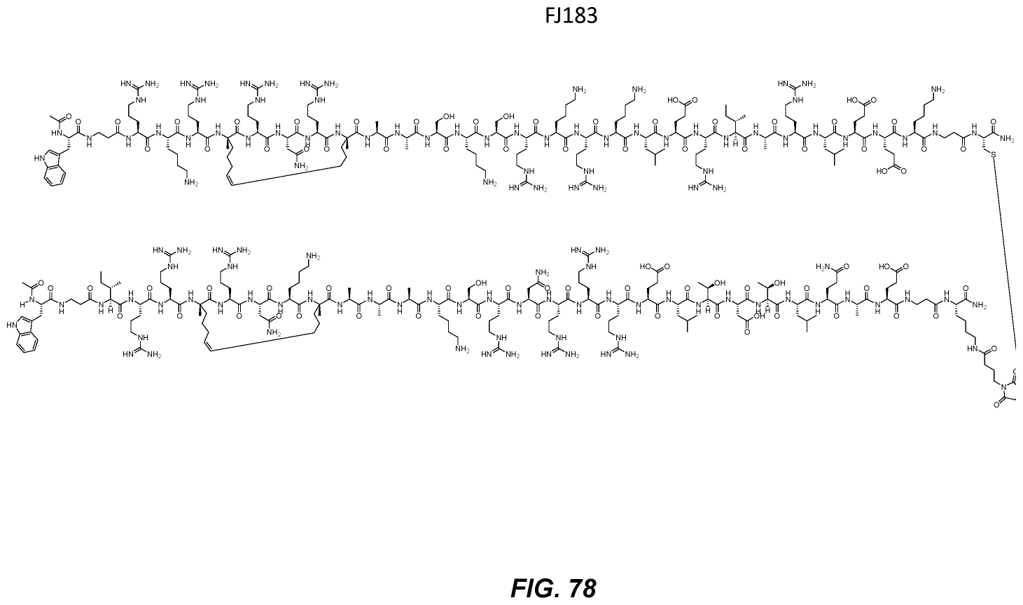

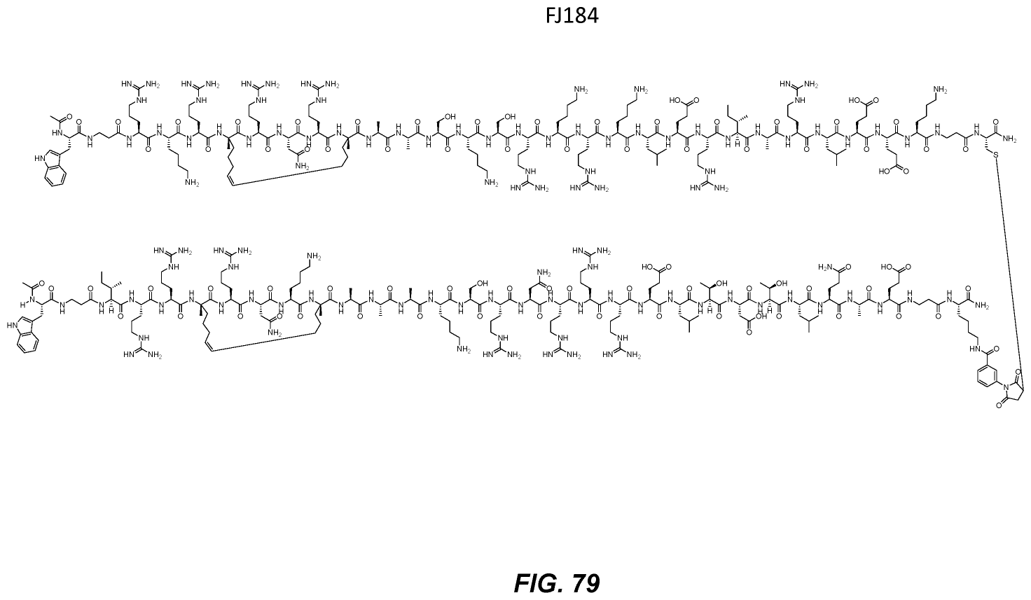

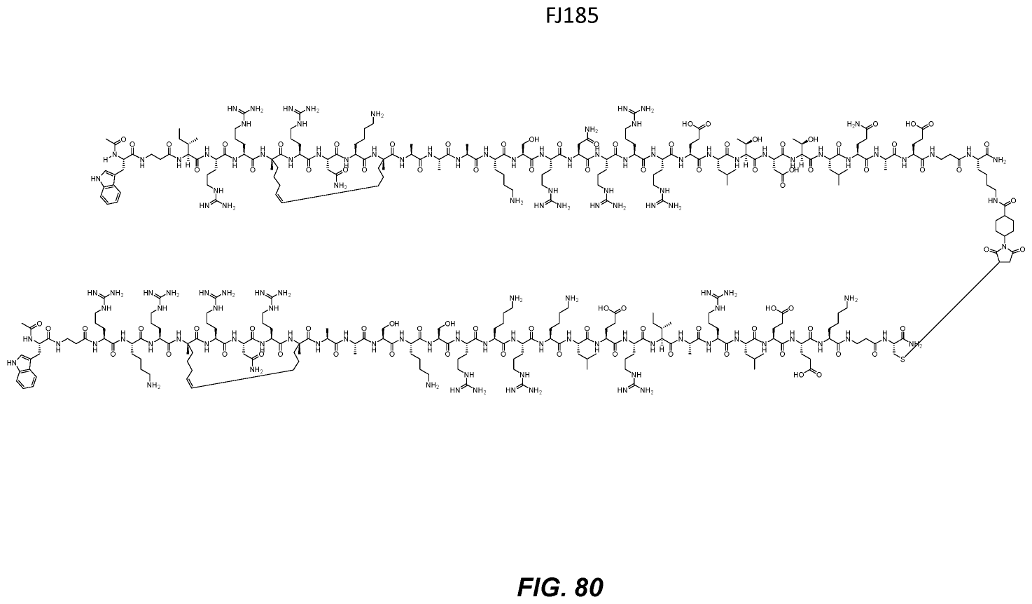

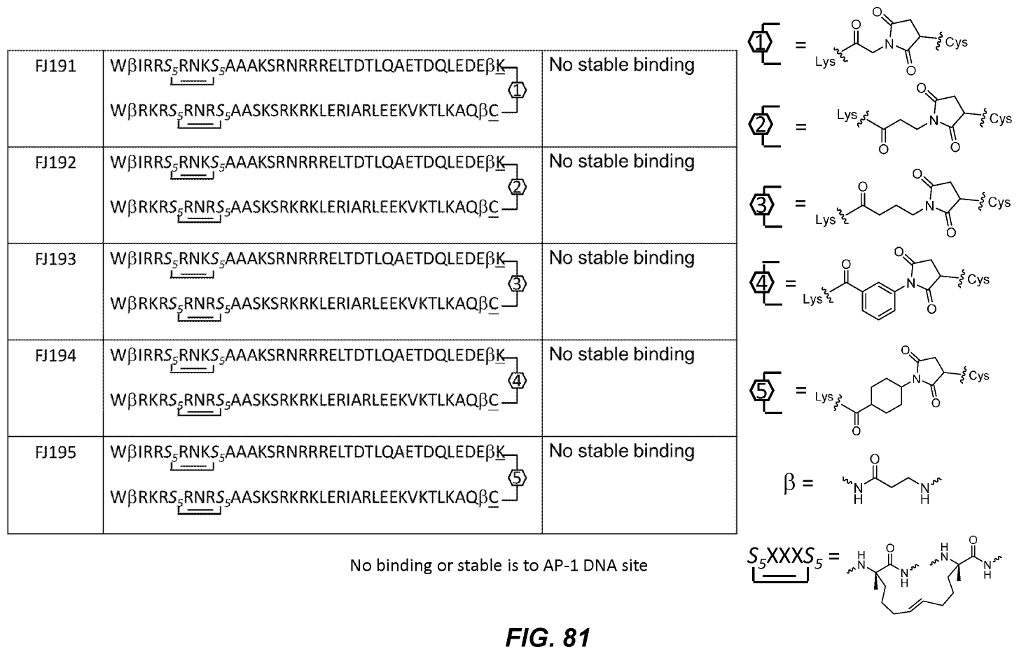

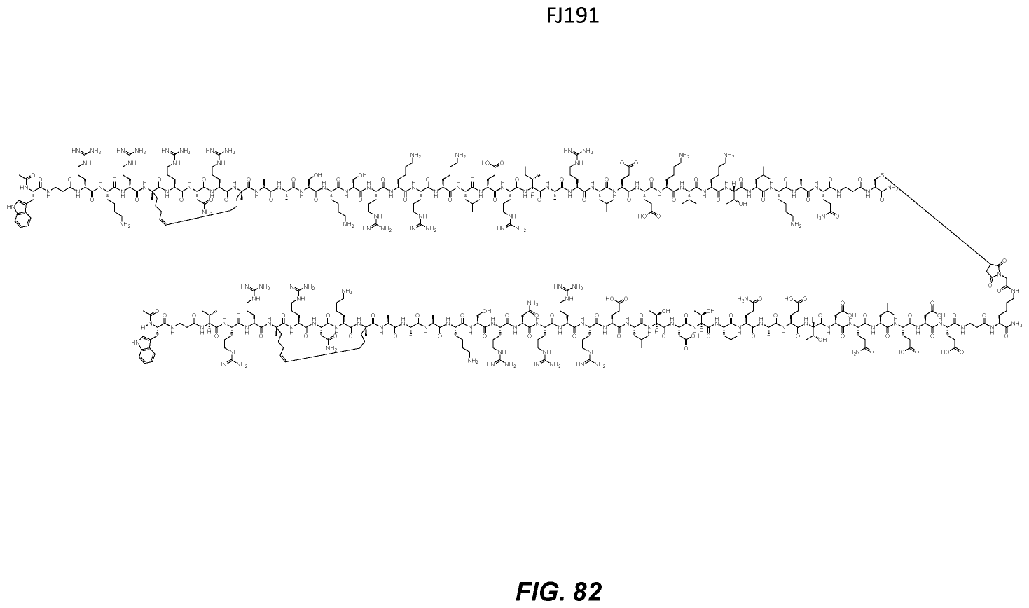

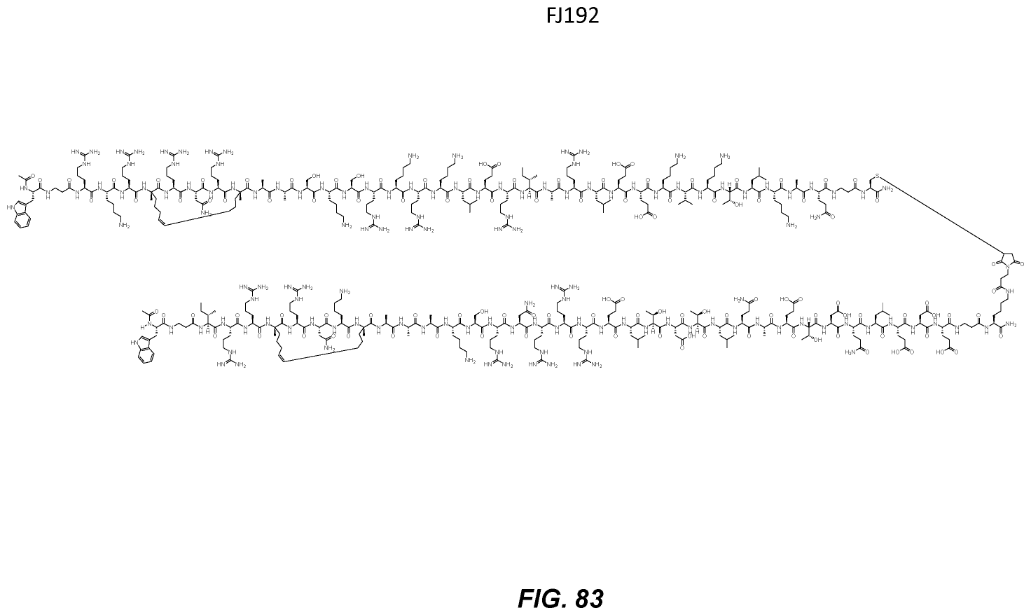

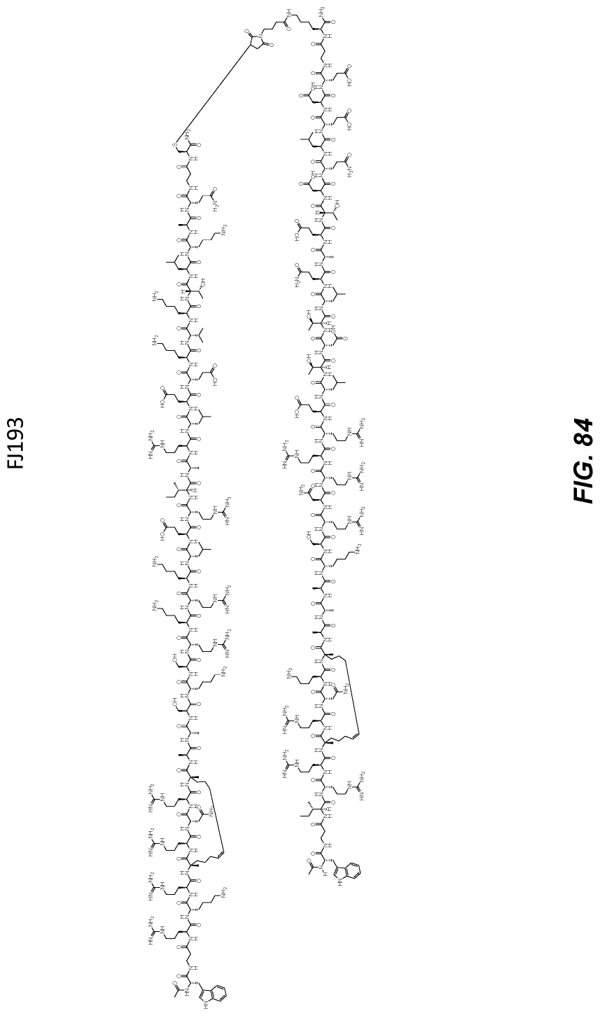

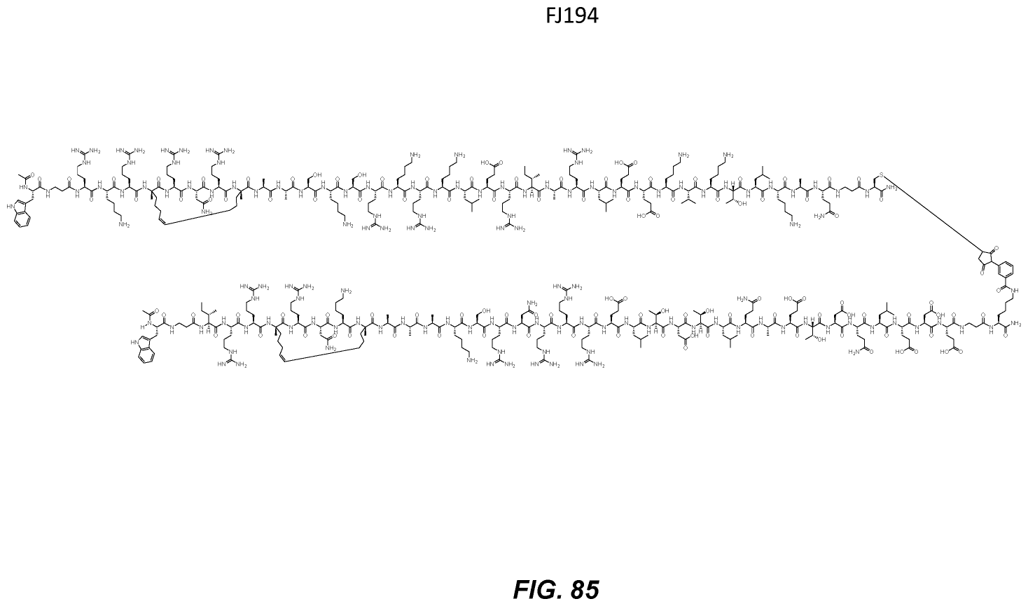

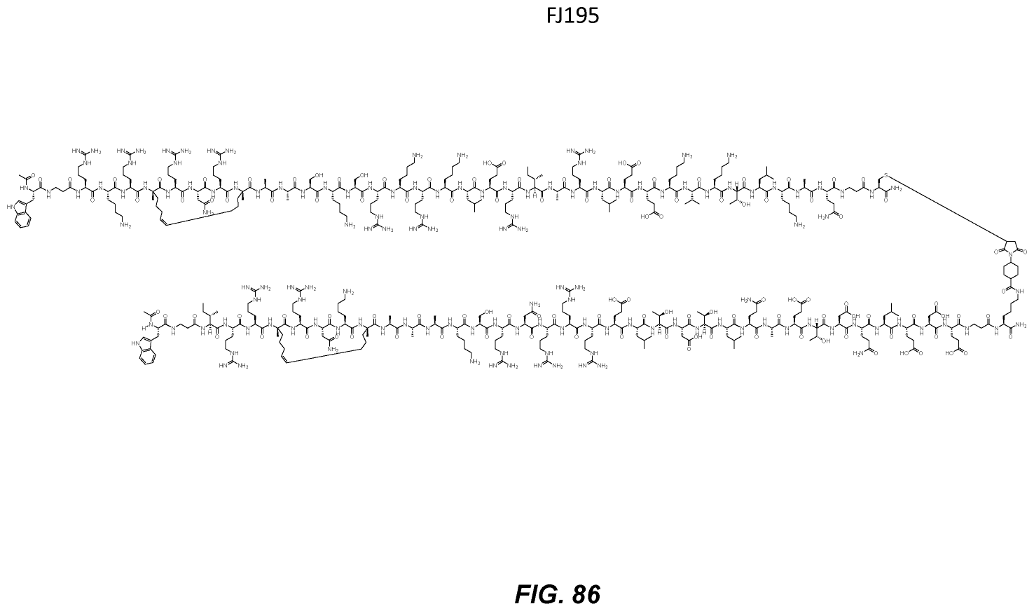

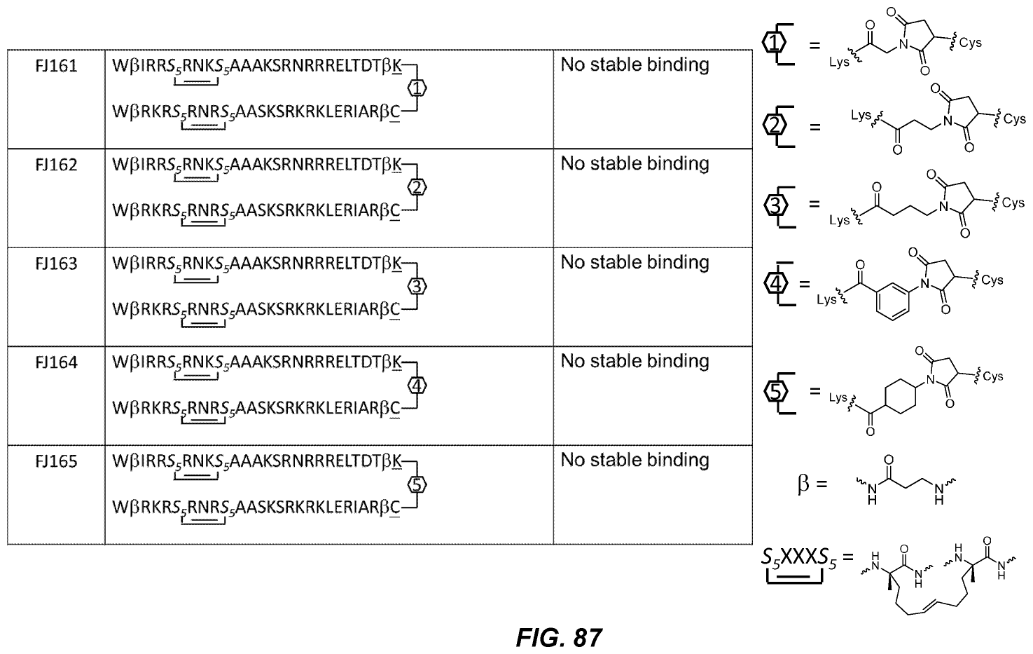

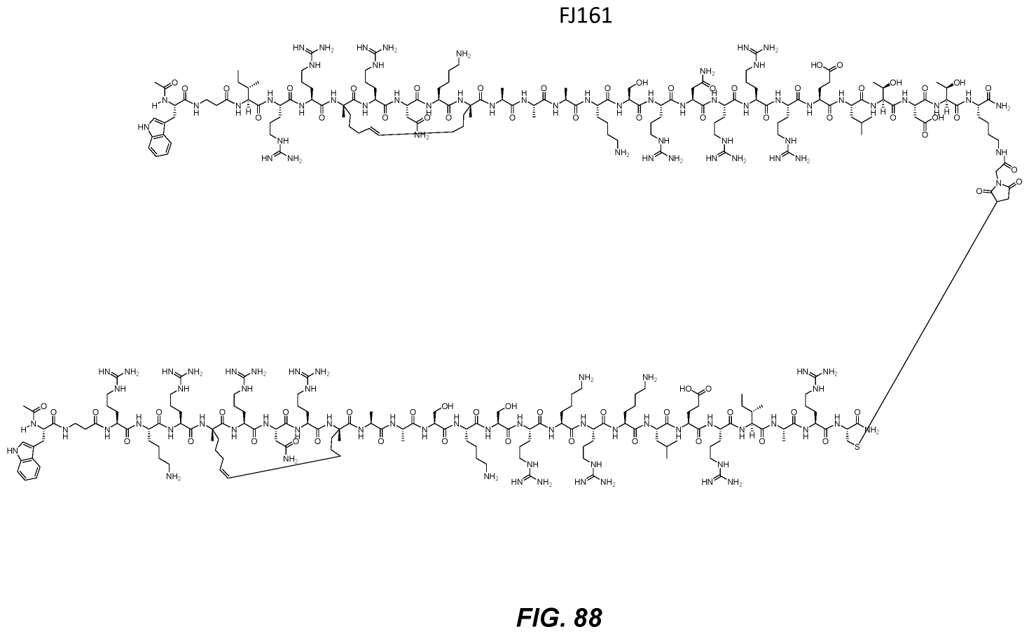

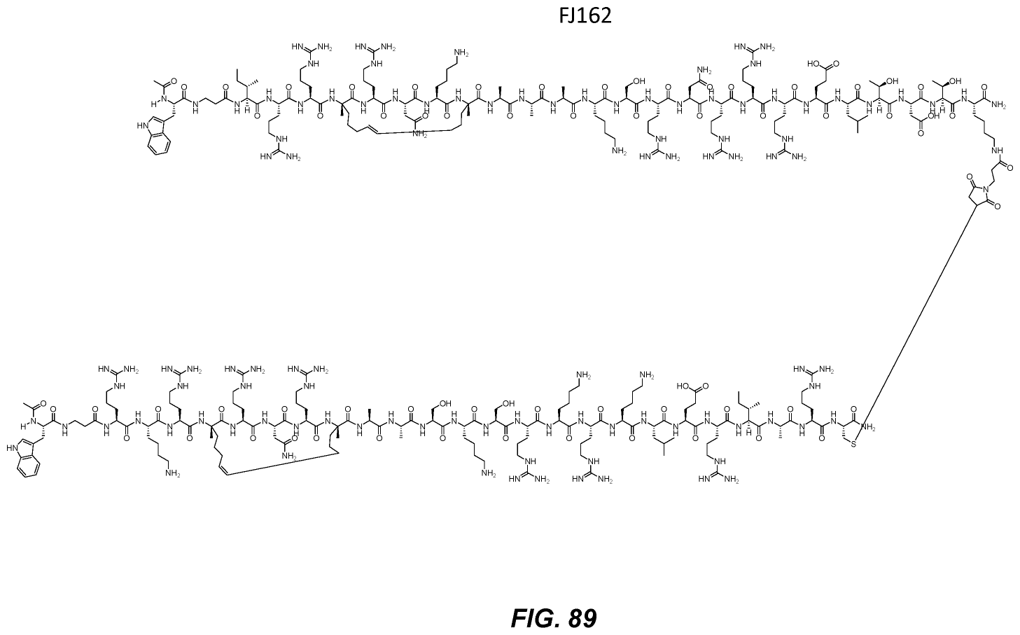

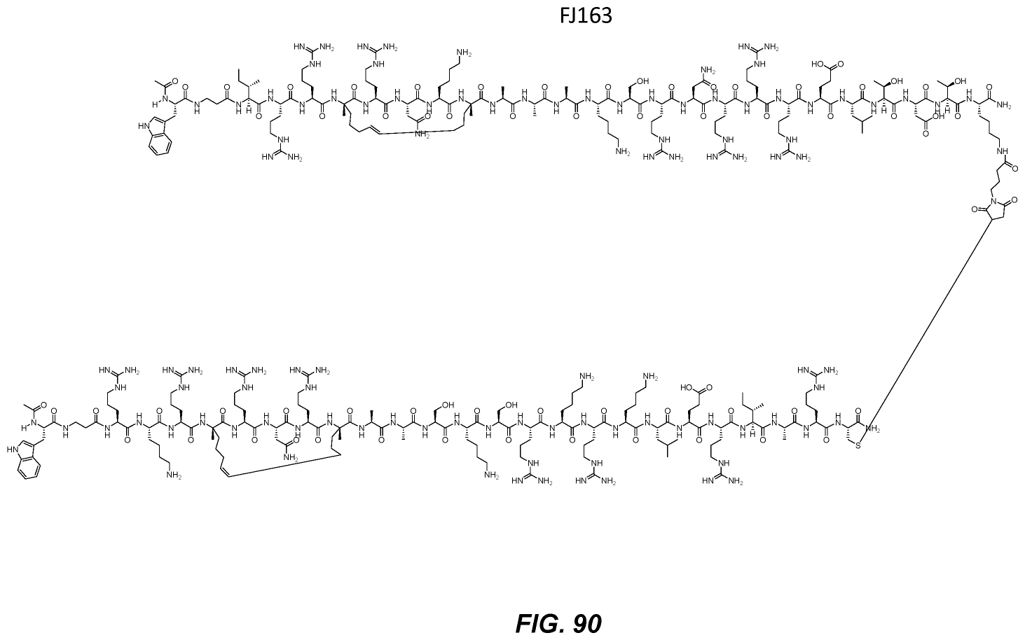

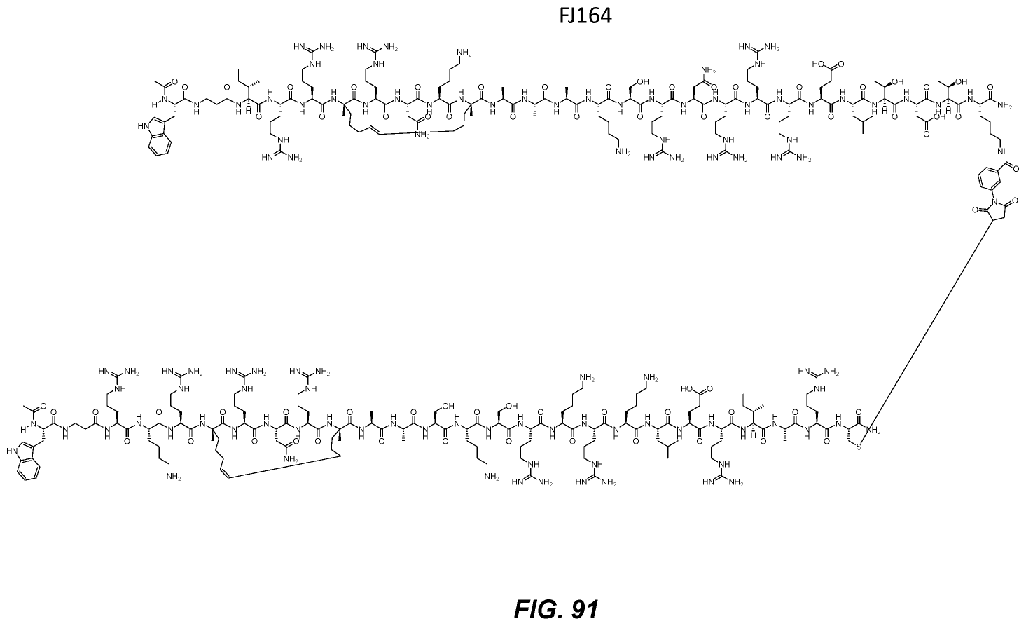

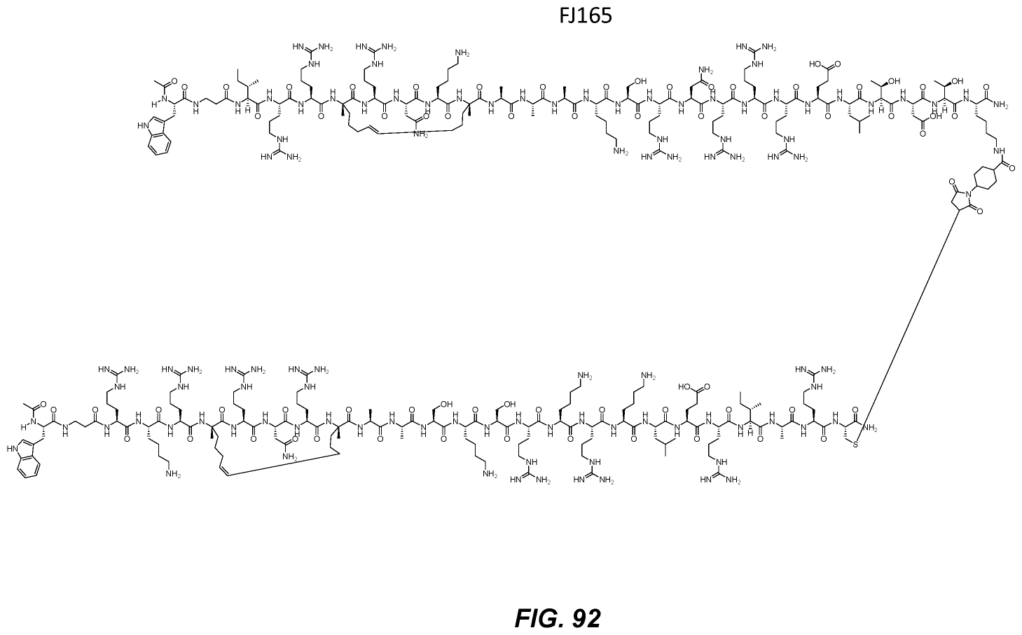

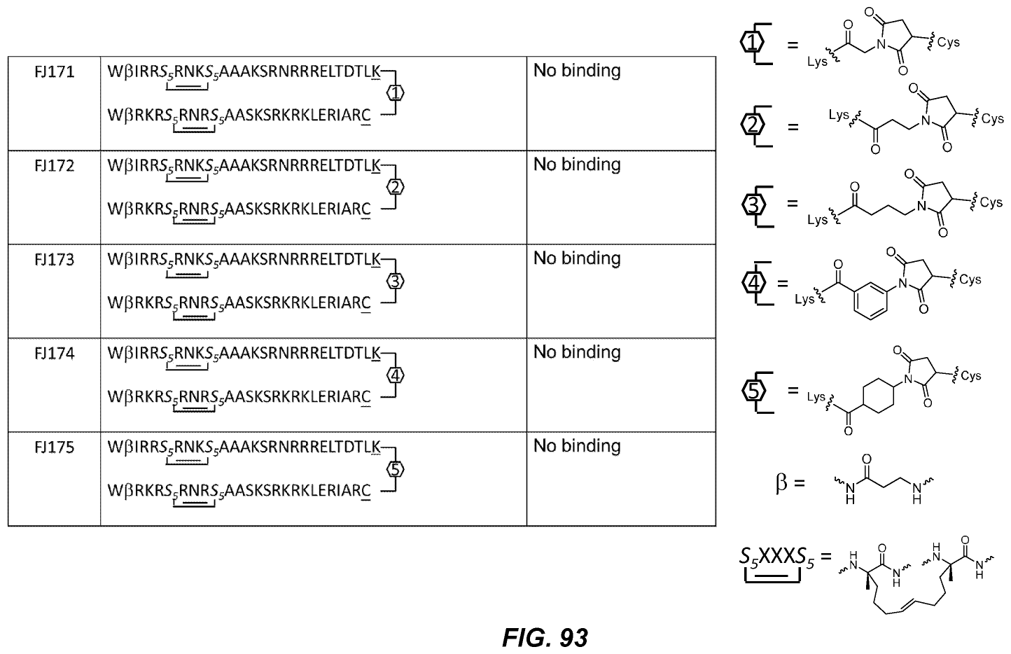

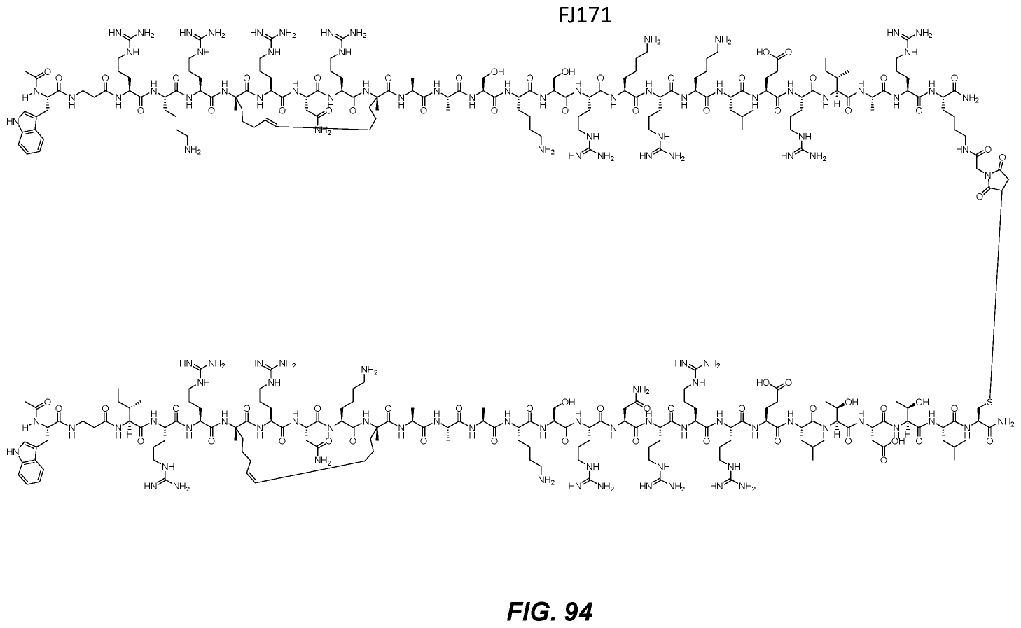

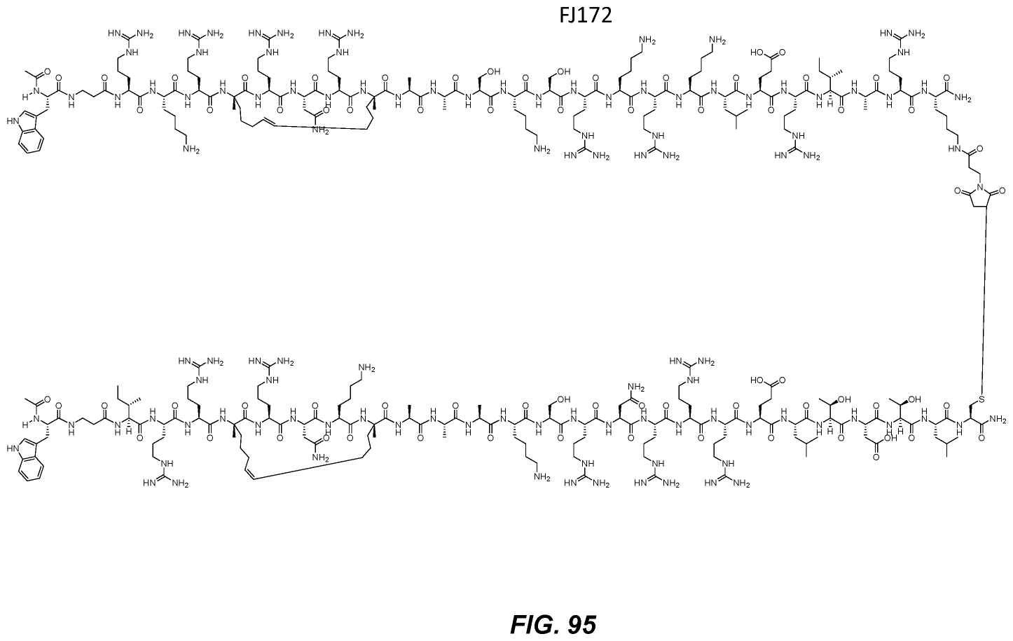

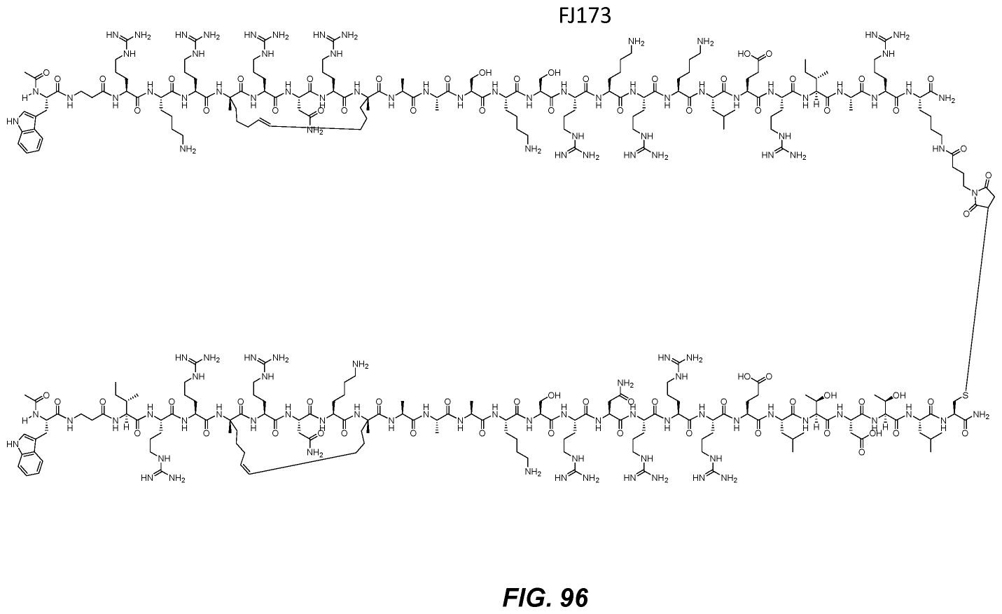

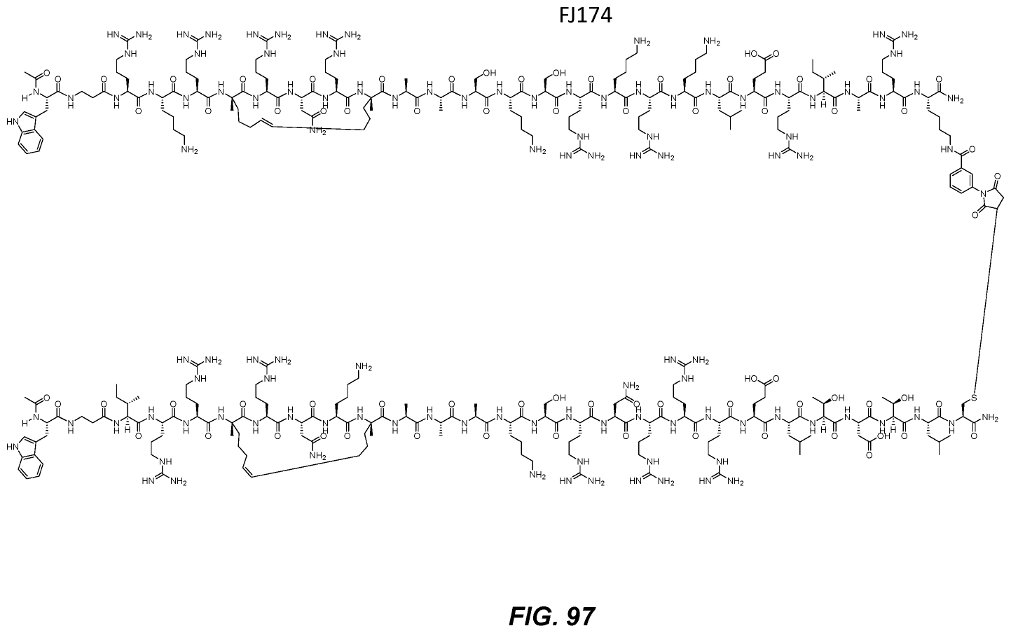

The DNA binding dimers shown in FIGS. 21-98 were generated by solid phase peptide synthesis of each peptide, followed by chemical linkage and, for stapled peptides, stapling via olefin metathesis. Each dimer was tested via electrophoretic mobility shift assay (EMSA) to determine DNA binding. In the tables of FIGS. 21, 28, 34, 37, 42, 49, 52, 57, 63, 69, 75, 81, 87, and 93, the first column indicates the dimer name, the second column provides sequence and structure information for the dimer, the third column provides comments regarding the dimer, and the fourth column provides affinity results from the EMSA.

### Example 4—DNA Binding, Cell Penetration, and Repression of Gene Expression by Synthetic Dimer STR4 and STR22

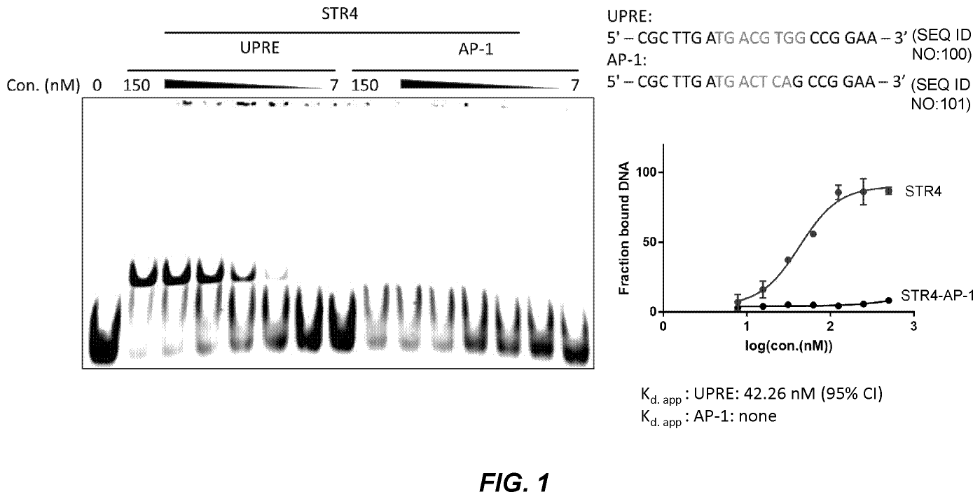

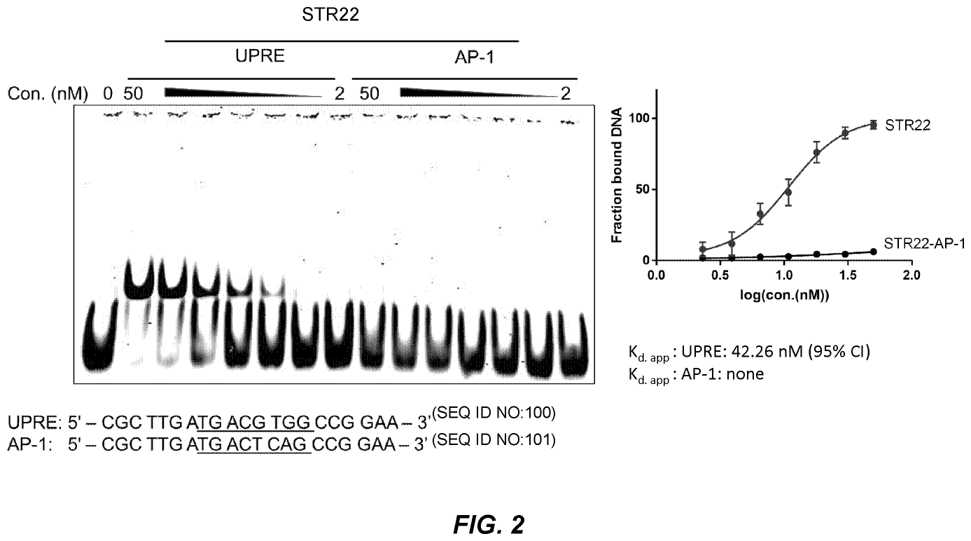

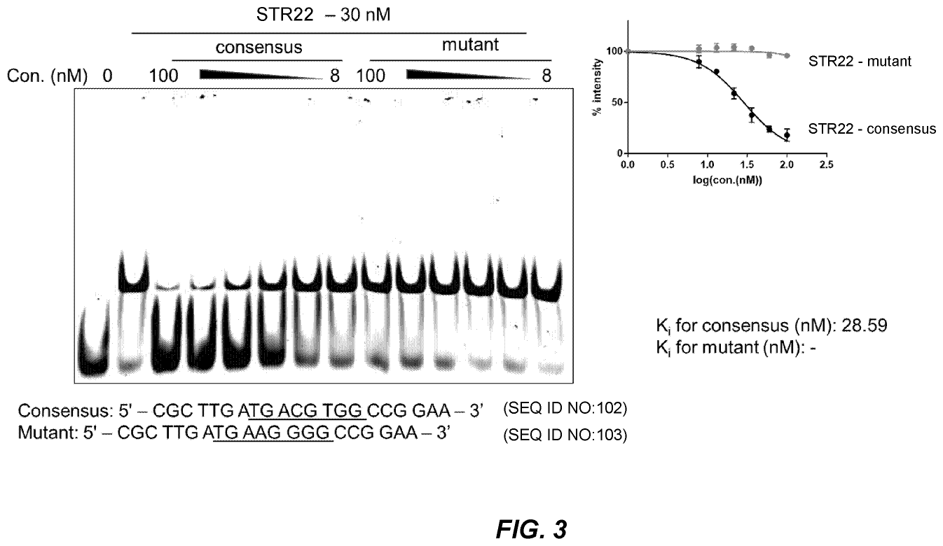

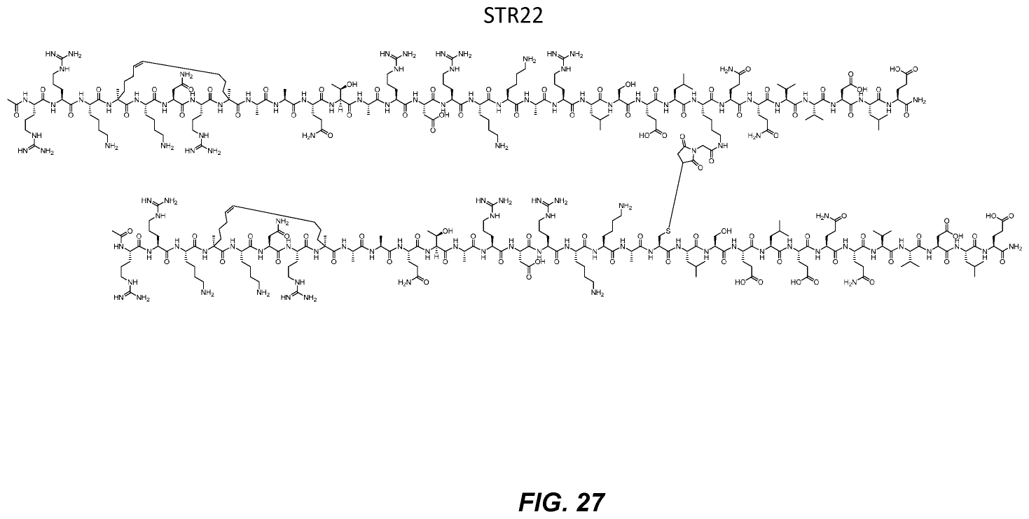

The DNA binding dimer STR22 (shown in FIG. 21 and FIG. 27) was synthesized. STR22 was subjected to EMSA to measure binding to either the UPR element (UPRE) or AP-1 binding site (AP-1). FIG. 1 shows STR4 binding at different concentrations, varying from 7 nM to 150 nM. FIG. 2 shows STR22 binding at different concentrations, varying from 2 nM to 50 nM. As shown in FIG. 2, the K_{D} against the UPRE was 14.17 nM. FIG. 3 shows binding of STR22 against either the consensus sequence or mutant sequence, as shown. The Kᵢ for the consensus sequence was 28.59 nM.

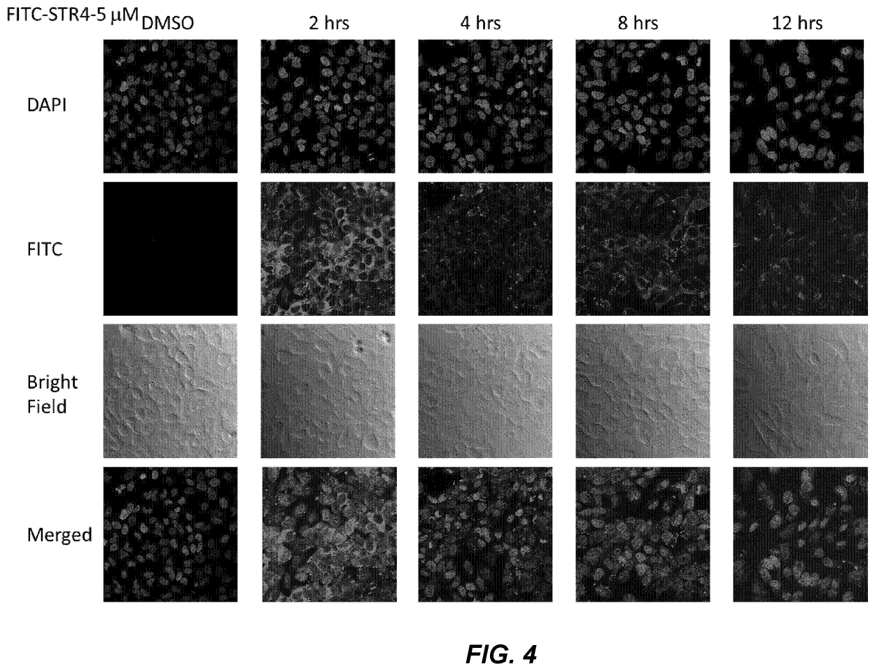

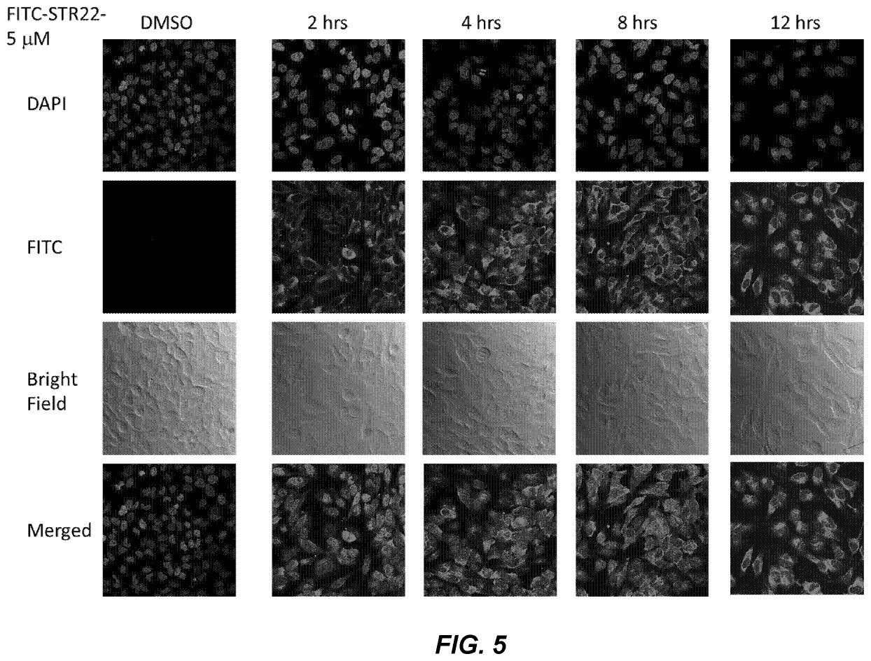

HeLa cells were treated with STR4-FITC (FIG. 4) or STR22-FITC (FIG. 5), at 5 μM for up to 24 hours. STR4-FITC and STR22-FITC both showed cell penetration, with intracellular STR4 peaking at 8 hours following treatment and decreasing thereafter. STR22 was retained at high levels for at least 24 hours following cell treatment.

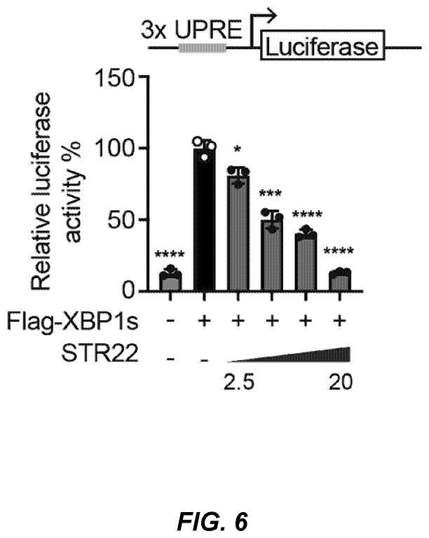

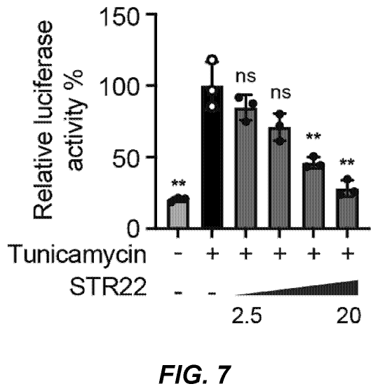

HeLa cells were co-transfected with XBP1 transcriptionally driven luciferase plasmid and renilla plasmid. 6 hours after transfection, cells were treated for 12 hours with tunicamycin at 500 ng/mL and either STR22 at varying concentrations (20, 10, 5, 2.5, 1.25 μM) or KIRA8 at 10 μM. As shown in FIG. 6 and FIG. 7, STR22 treatment inhibited tunicamycin-induced luciferase expression at all concentrations shown.

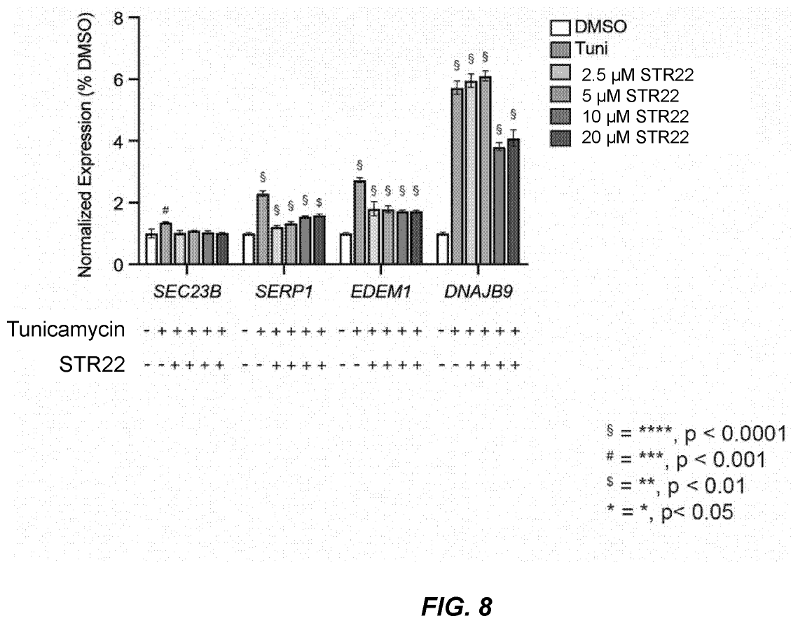

HeLa cells were treated with STR22 at varying concentrations (2.5, 5, 10, 20 PM) for 36 hours; 24 hours into the STR22 treatment, tunicamycin was added for an additional 12 hours at 5000 ng/ml. SEC23B, SERP1, EDEM1, and DNAJB9 expression were measured with mRNA-qPCR. As shown in FIG. 8, STR22 pre-treatment reduced tunicamycin-induced gene expression in the targets analyzed.

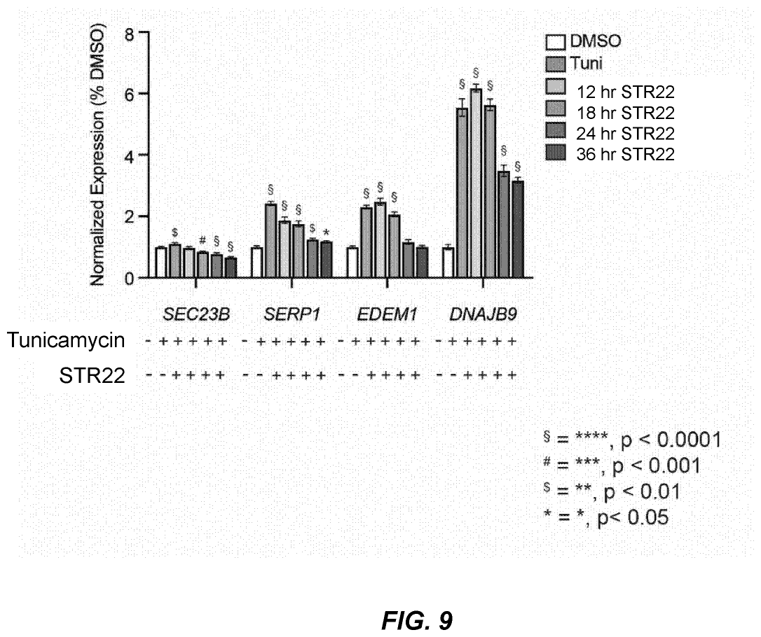

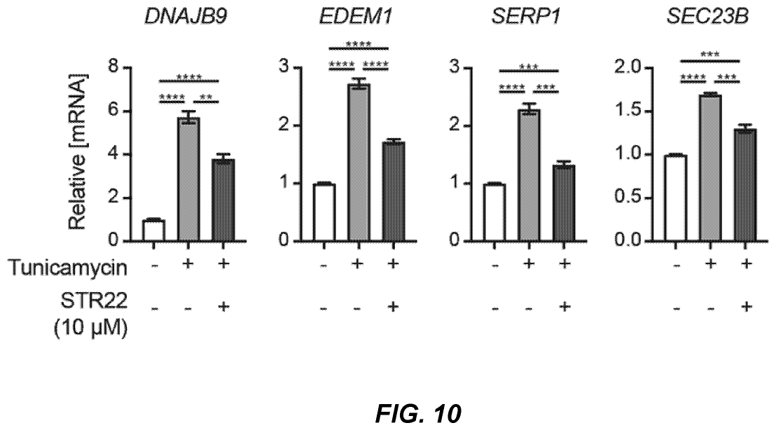

HeLa cells were treated with STR22 at 20 μM for varying times (12, 18, 24, or 37 hours); for the last 12 hours of treatment, tunicamycin was added at 5000 ng/ml. SEC23B, SERP1, EDEM1, and DNAJB9 expression were measured with mRNA-qPCR. As shown in FIG. 9, gene expression was reduced further with greater amounts of STR22 treatment time.

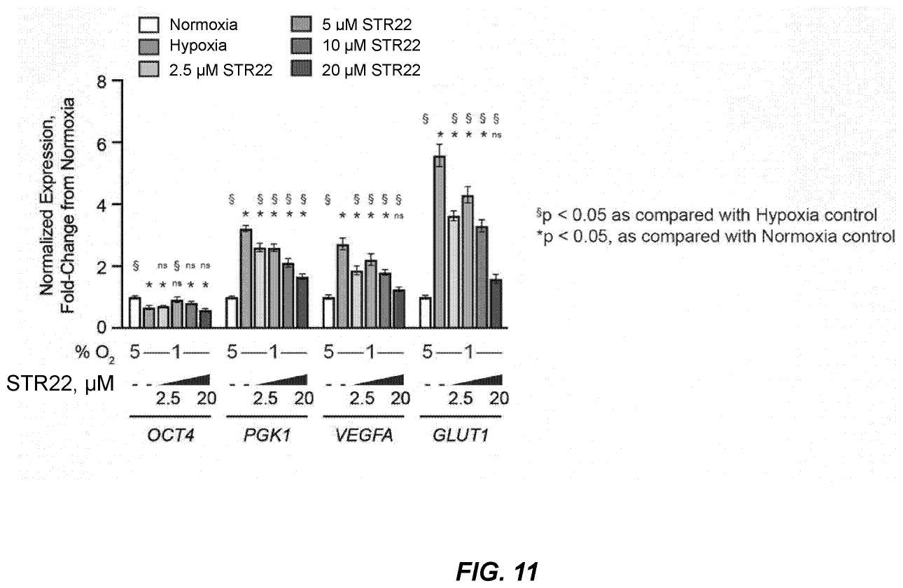

HeLa cells were treated with STR22 at varying concentrations (2.5, 5, 10, 20 PM) for 48 hours; 24 hours into the STR22 treatment, cells were exposed to either normoxia (5% 02) or hypoxia (1% 02) for the additional 24 hours. Expression of OCT4, PGK1, VEGFA, and GLUT1 were measured with mRNA-qPCR. As shown in FIG. 11, STR22 treatment reduced gene expression of target genes under hypoxic conditions.

### Example 5—Synthetic Transcriptional Repressors Inhibit HIF1α DNA Binding and Target Gene Expression

Both XBP1 and HIF1α are strongly upregulated in triple negative breast cancer (TNBC) and are required for tumor cell growth and survival in a variety of preclinical TNBC models. HIF1α is overexpressed in TNBC and has been shown to correlate with tumor size. Genetically silencing HIF1α led to substantial reduction in the growth of human TNBC xenografts, and a hypoxic gene signature based upon HIF1α-regulated genes showed association with poor patient outcome. Analysis of independent cohorts of TNBC patients identified a specific XBP1 gene expression signature that tightly correlates with HIF1α expression and the hypoxic response as well as poor patient prognosis. Together, these data strongly implicate XBP1 and HIF1α as key transcriptional drivers in TNBC. Yet, there are currently no pharmacologic agents available to target these transcriptional factors individually or in combination.

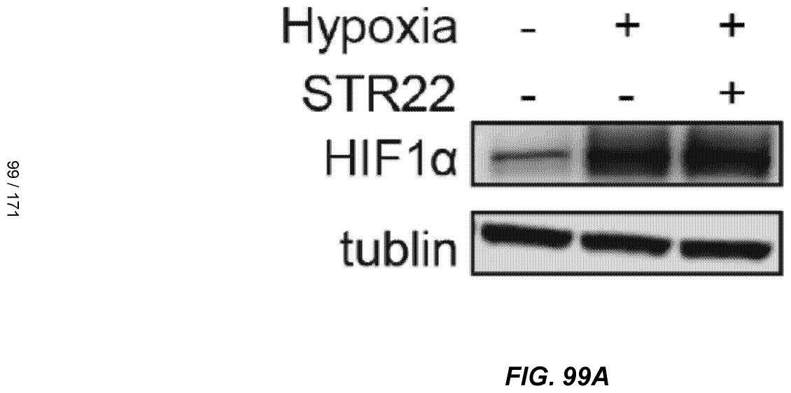

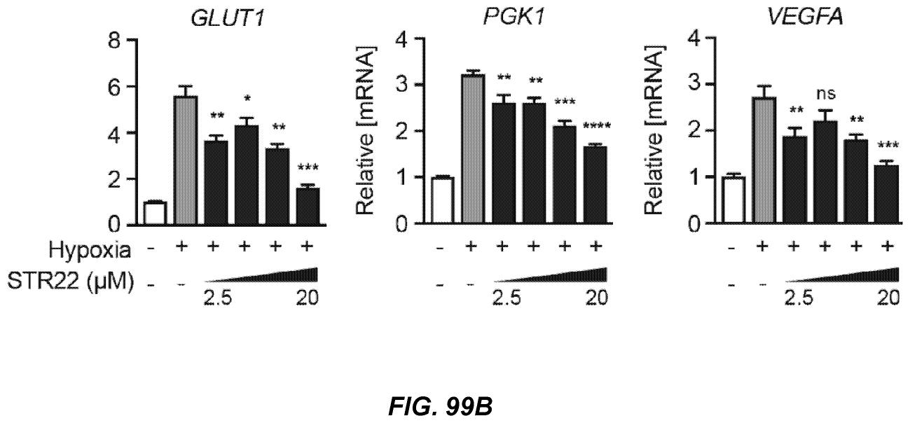

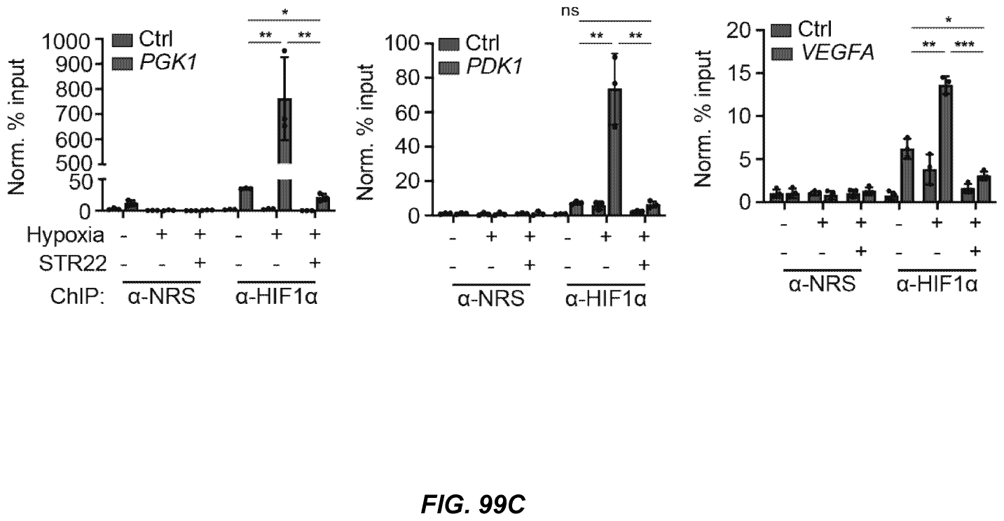

To study the effects of STR22 on hypoxia-induced gene transcription factor DNA binding and target gene expression, the induction of HIF1α and downstream HRE-target genes like VEGFA, PDK1, PGK1 and GLUT1 in response to acute hypoxia (e.g., 1% 02 for 6 hr) was validated. Treatment of hypoxic HeLa and MDA-MB-231 TNBC cells with STR22 did not affect the induction of HIF1α protein (FIG. 99A), but significantly inhibited expression of downstream HRE-regulated target gene mRNAs (FIG. 99B). It was confirmed by ChIP-qPCR that HIF1α binding at target HRE-promoters in these genes is drastically increased under hypoxic conditions. Remarkably, STR22 treatment almost completely blocked HIF1α binding at these sites, presenting direct evidence of TF-DNA inhibition in cells (FIG. 99C).

### Example 6—Synthetic Transcriptional Repressors Inhibit Hypoxic Gene Expression and Invasion in TNBC Cells

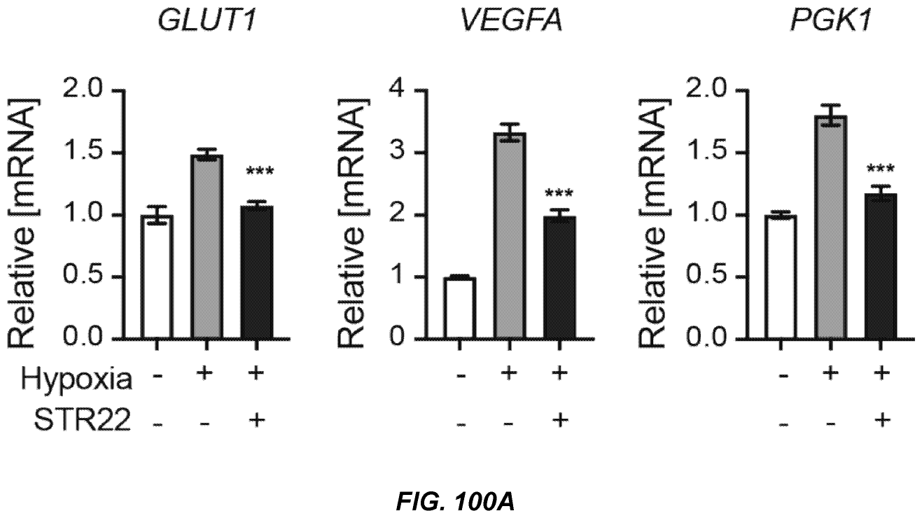

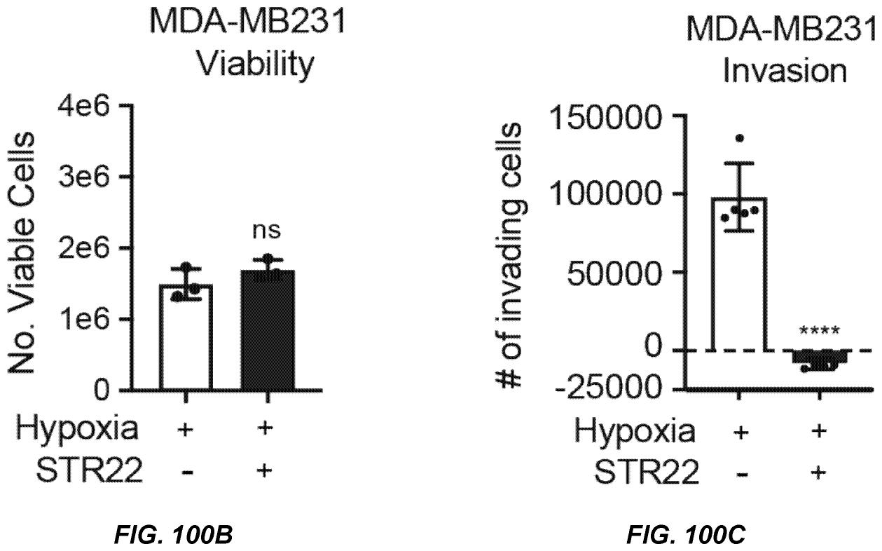

The effect of STR22 on cell growth and invasion in culture under normoxic or hypoxic conditions was determined. TNBC cells (MDA-MB-231) were grown under normoxic conditions, STRs or vehicle were added, and the cells were either left under normoxic conditions (20% oxygen) or transferred to hypoxia chambers (1% oxygen). qPCR analysis of hypoxia-induced genes GLUT1, VEGFA, and PGK1 demonstrated reduction in hypoxia-induced gene expression with STr22 treatment (FIG. 100A). Treatment of MDA-MB231 cells with 20 μM STR22 for 24 hours did not impact cell viability (FIG. 100B) but significantly inhibited cell invasion (FIG. 100C).

### Example 7—Analysis of DNA Binding of Additional bZIP Transcriptional Repressors

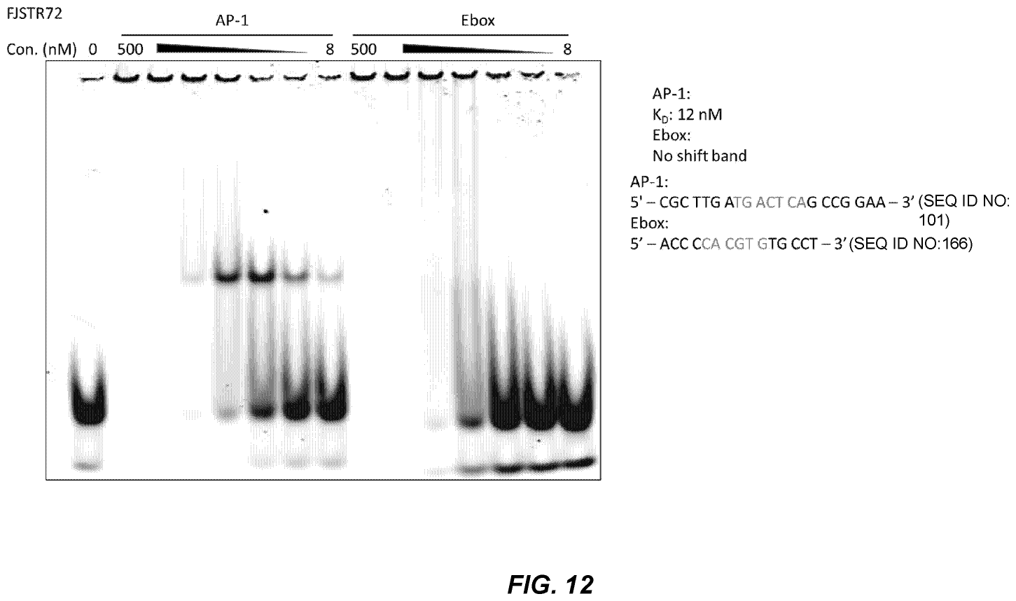

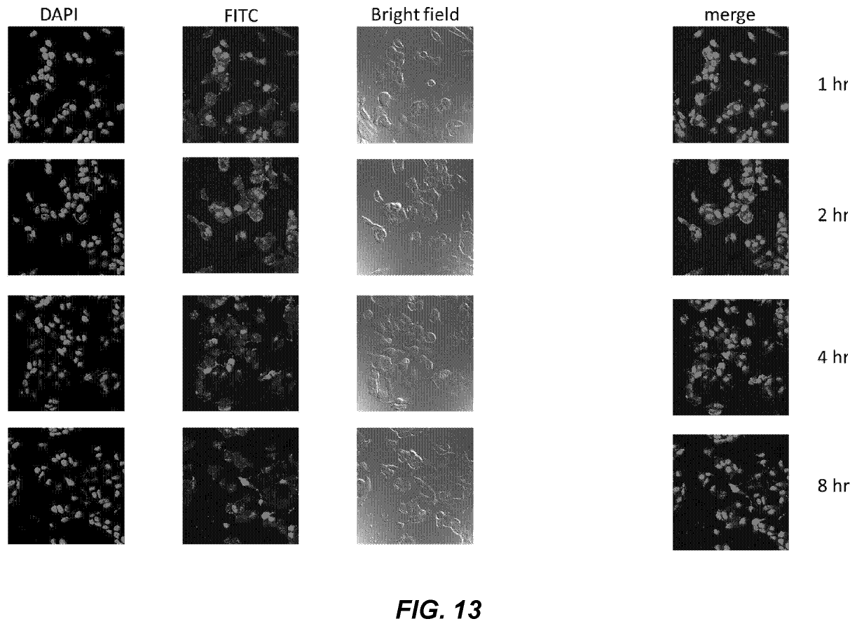

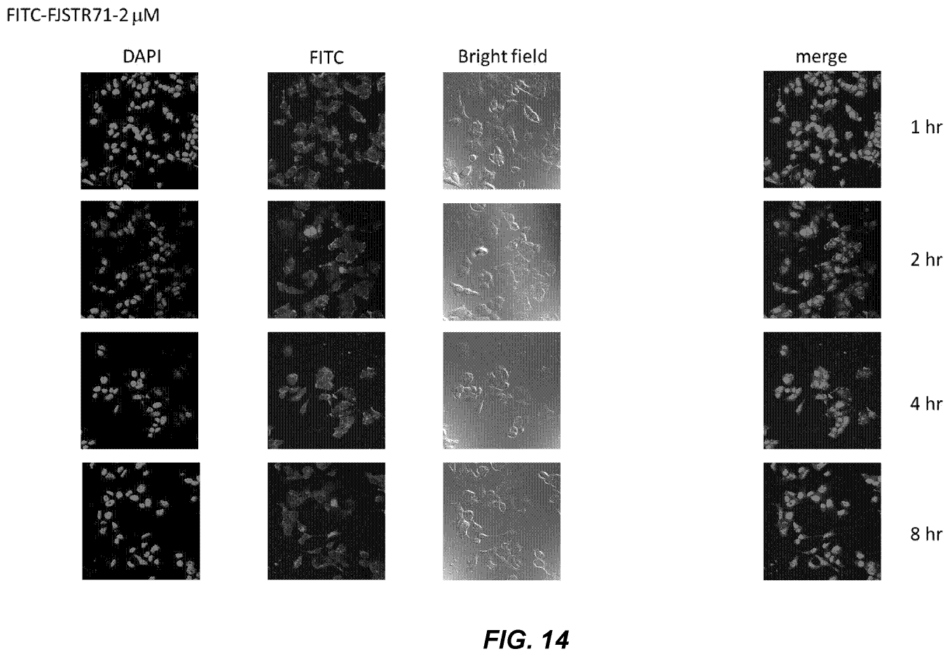

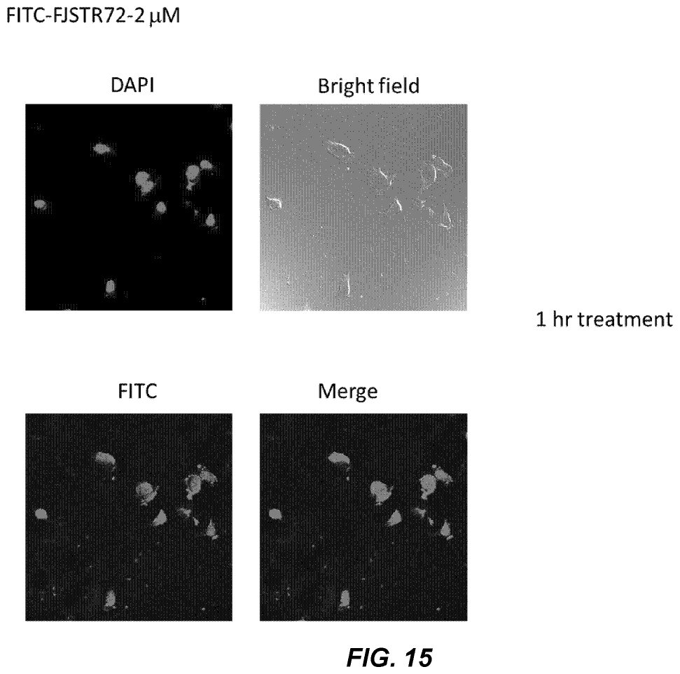

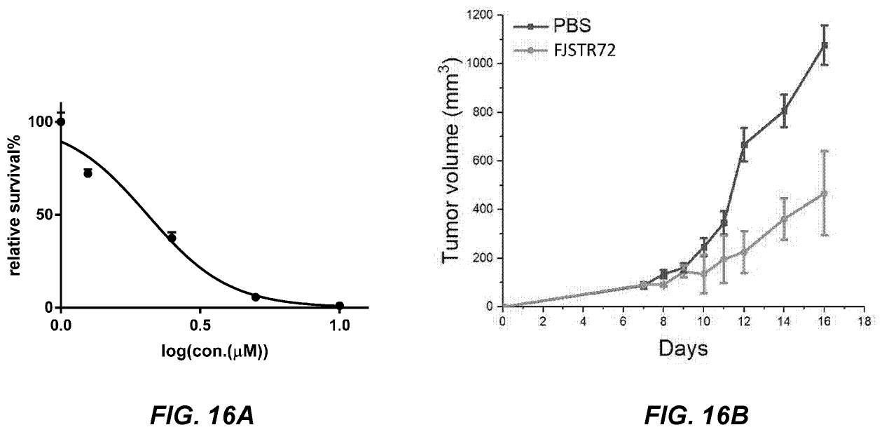

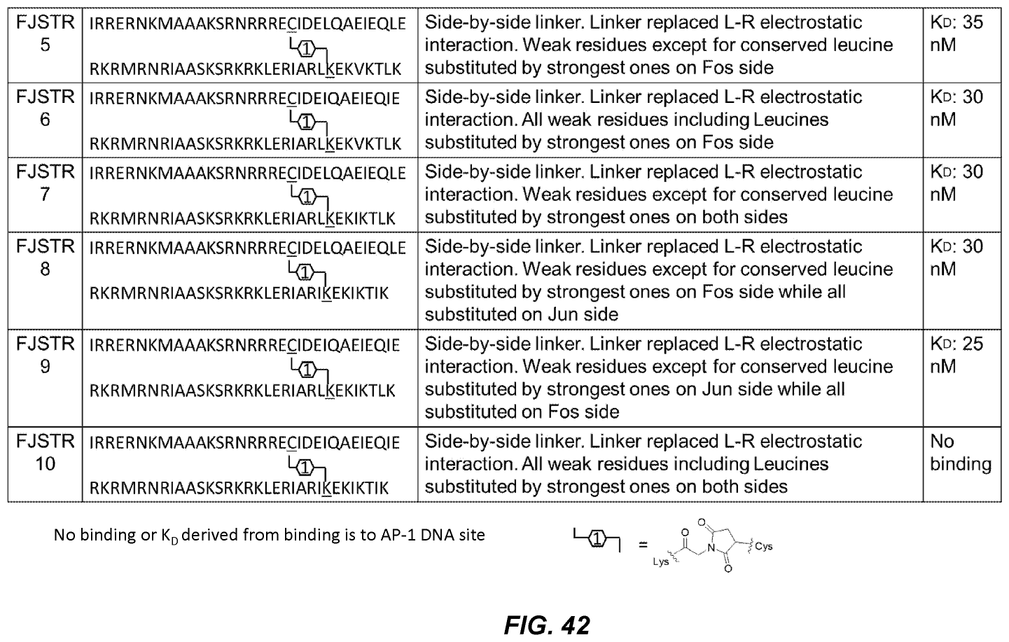

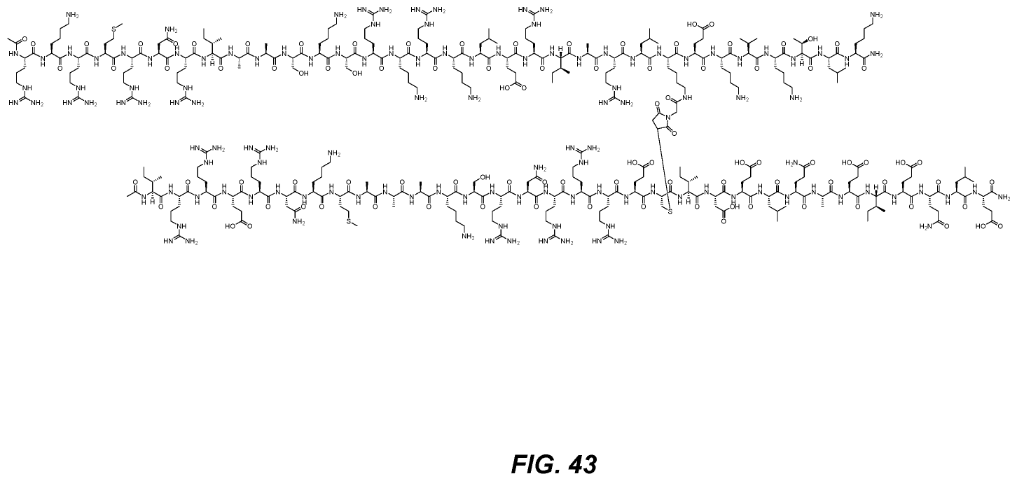

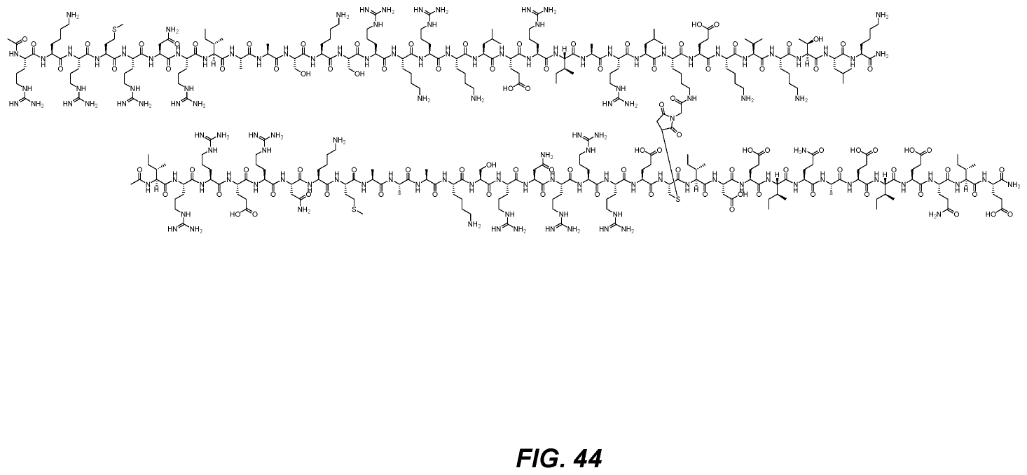

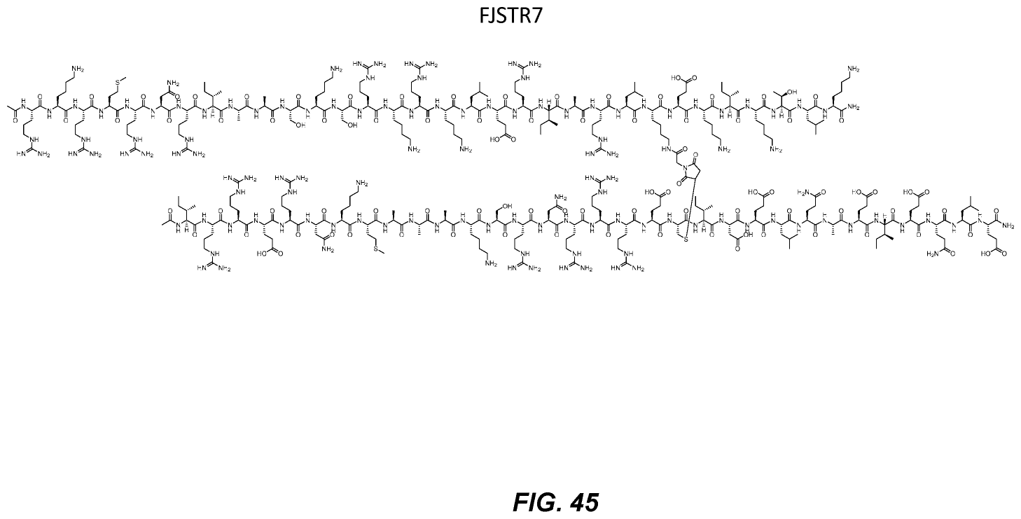

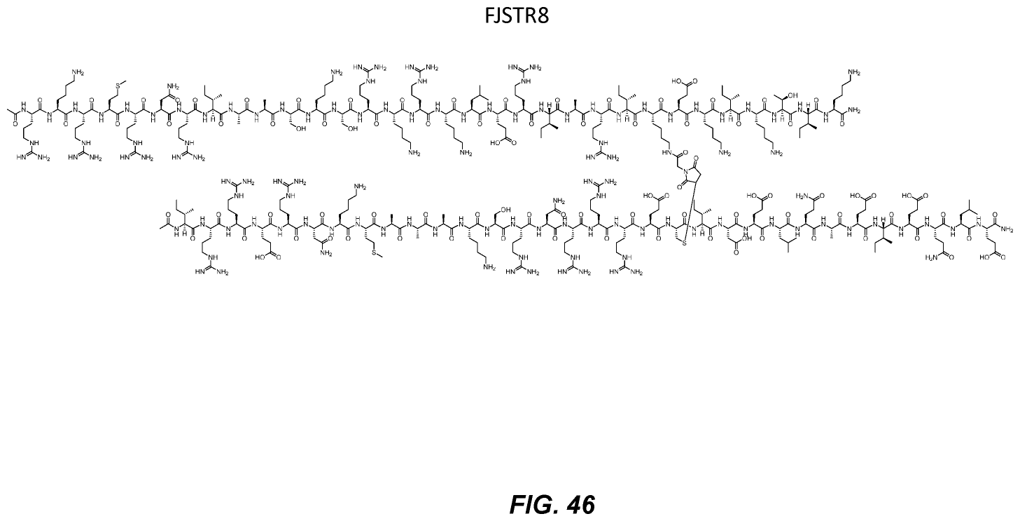

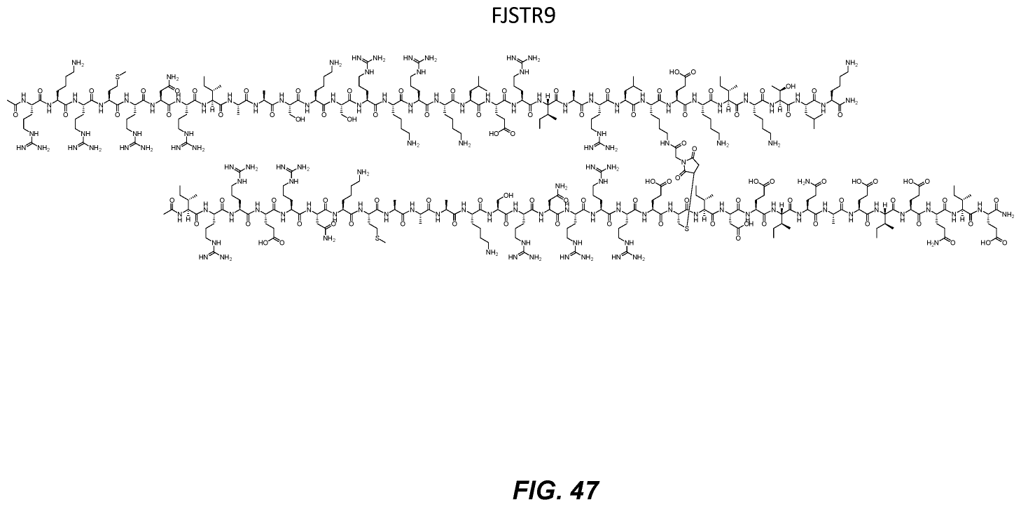

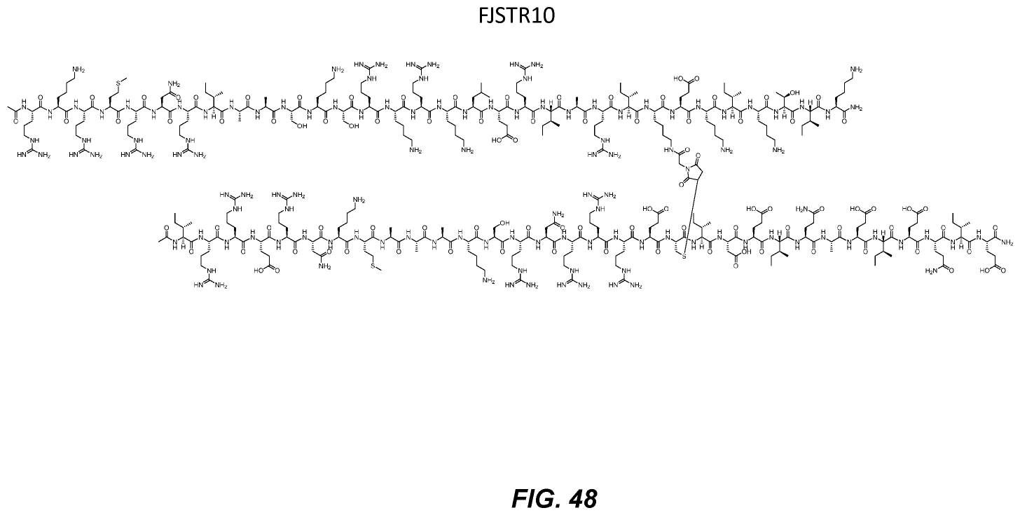

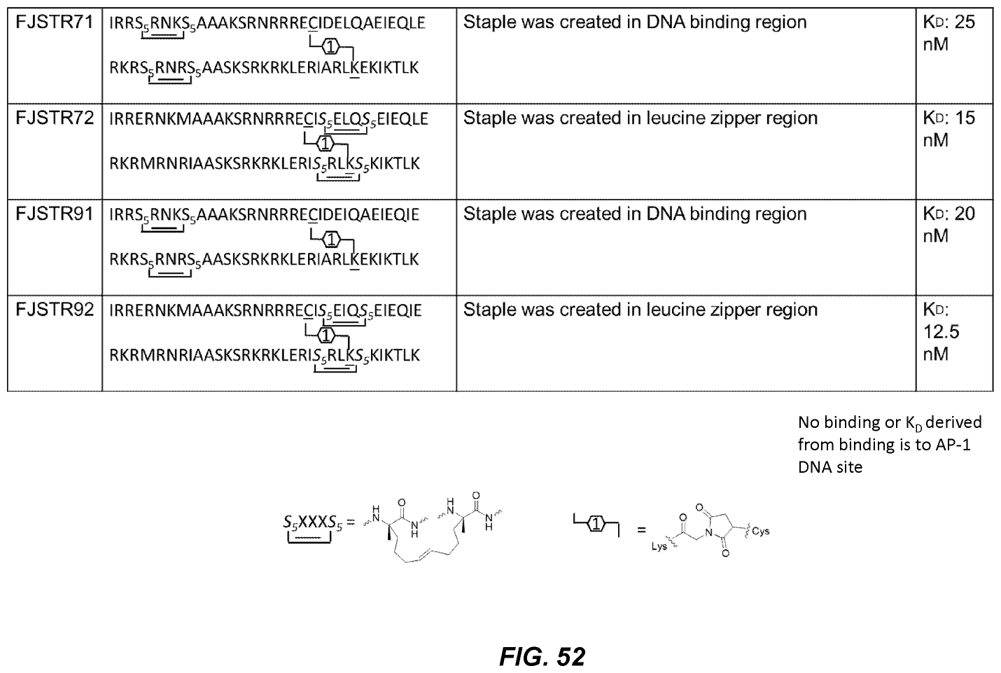

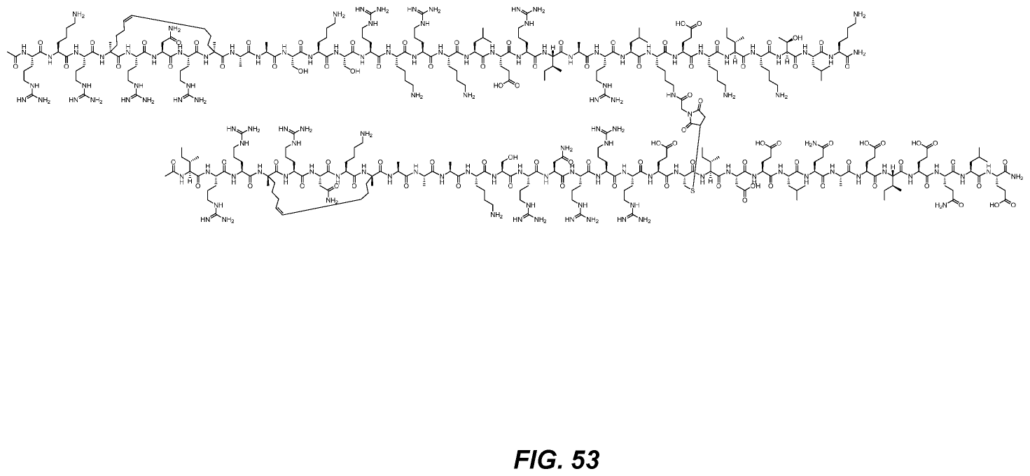

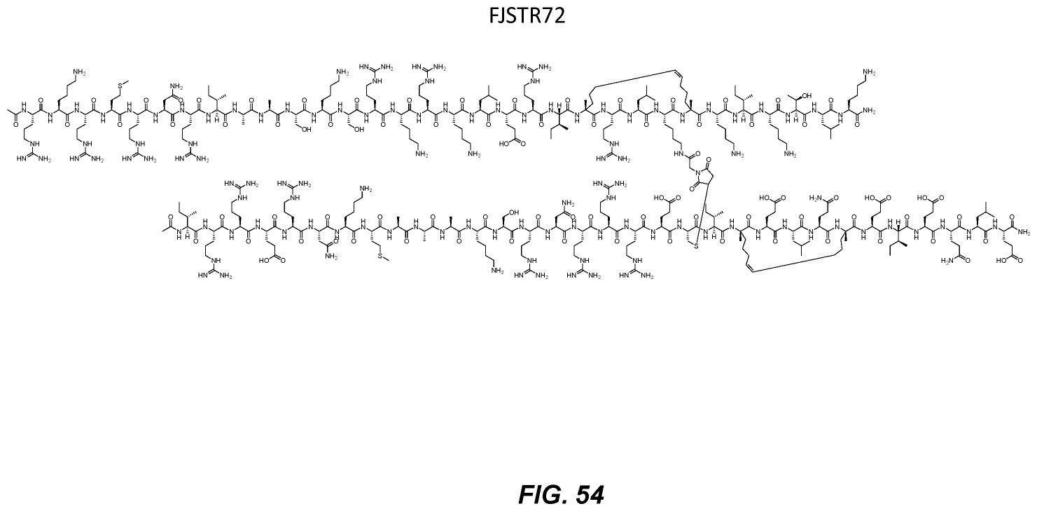

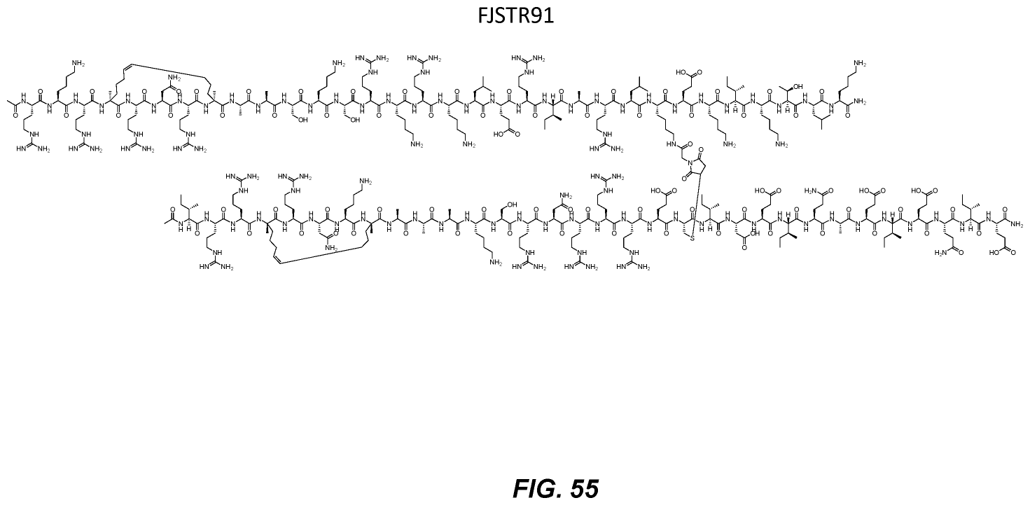

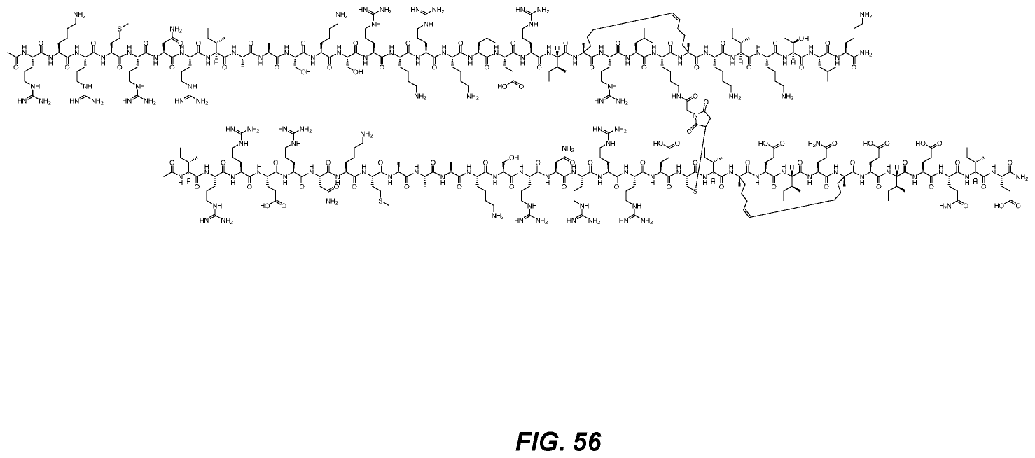

bZIP transcriptional repressors FJSTR7 (shown in FIGS. 42 and 45), FJSTR71 (shown in FIGS. 52 and 53), and FJSTR72 (shown in FIGS. 52 and 54), were generated and tested. As shown in FIG. 12, FJSTR72 binds to the AP-1 site with a K_{D} of 12 nM, but does not bind to the Ebox site. As shown in FIGS. 14-16, FJSTR7, FJSTR71, and FJSTR72 are all capable of entering cells. As shown in FIG. 16, intratumoral treatment with FJSTR72 reduces tumor growth in a MC38-bearing C57BL/8 mouse model.

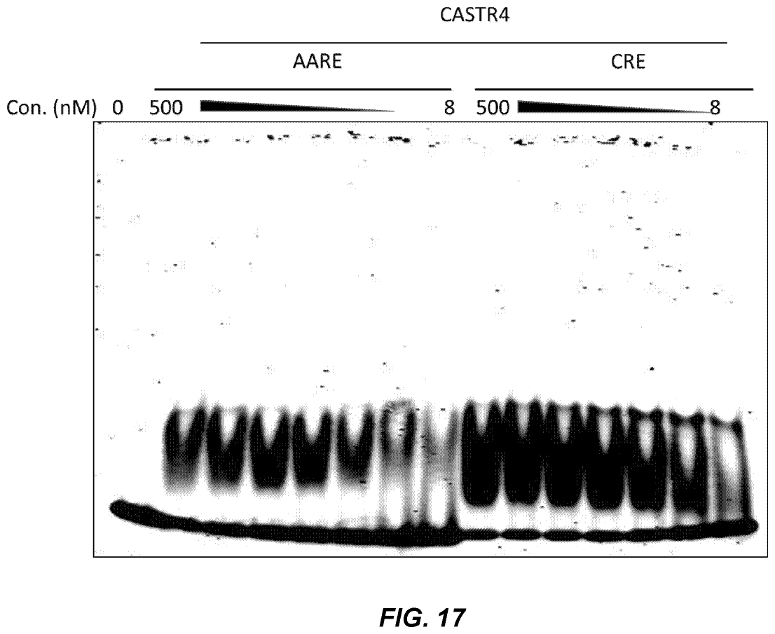

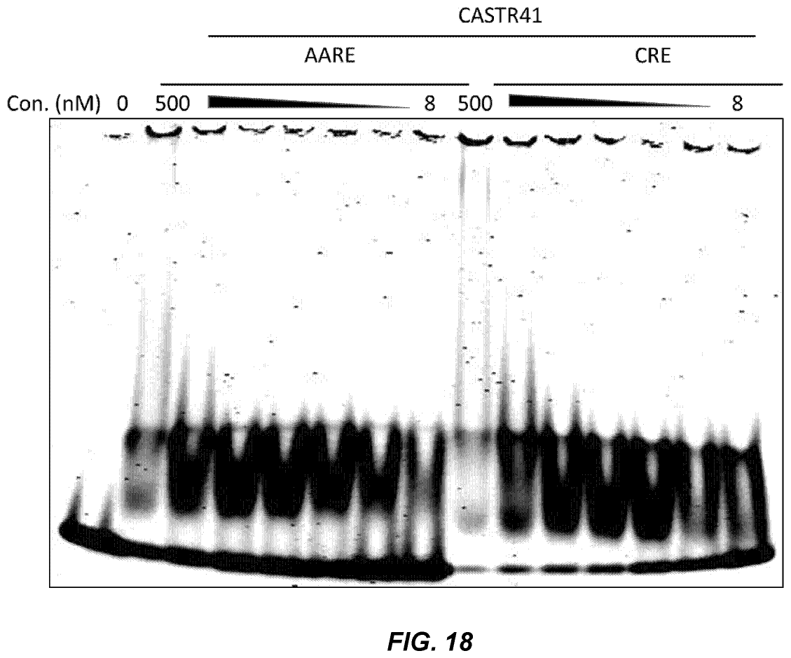

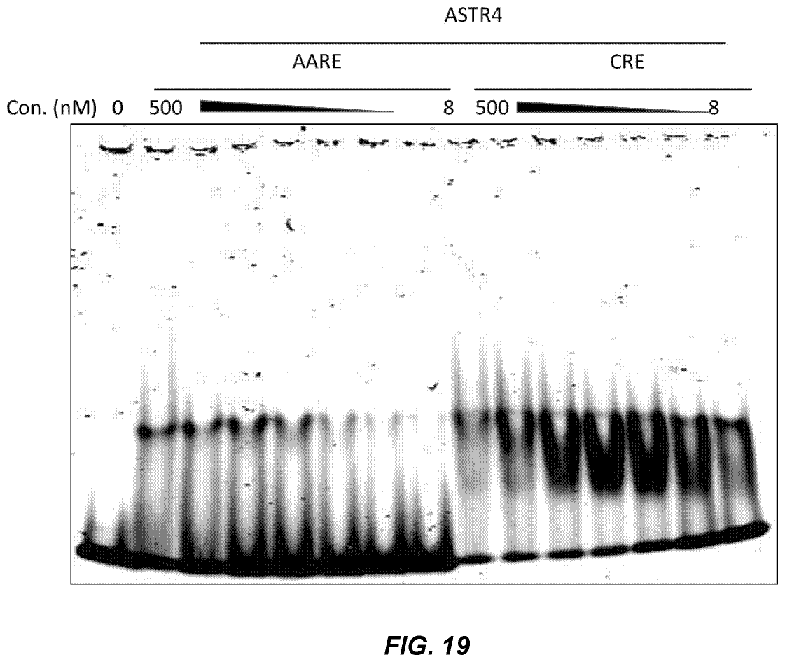

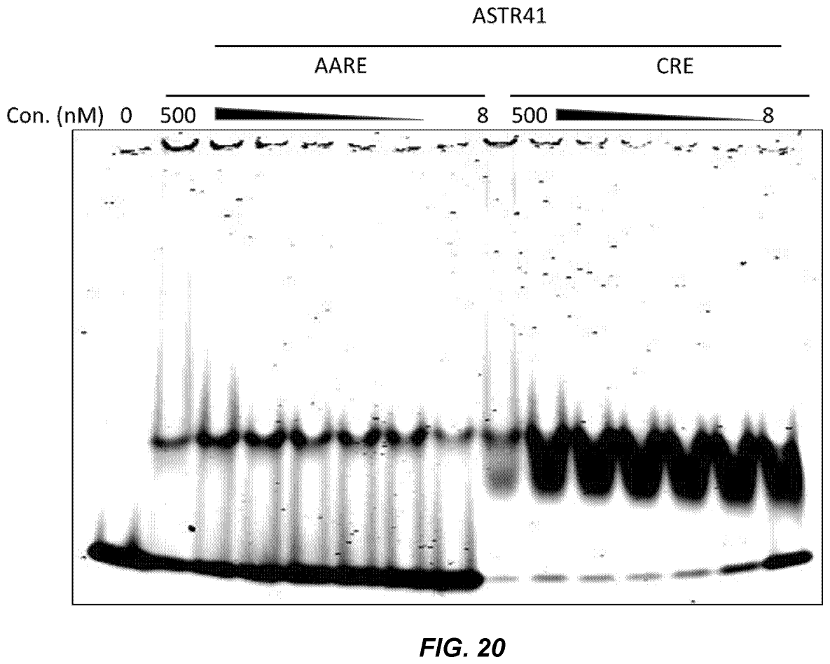

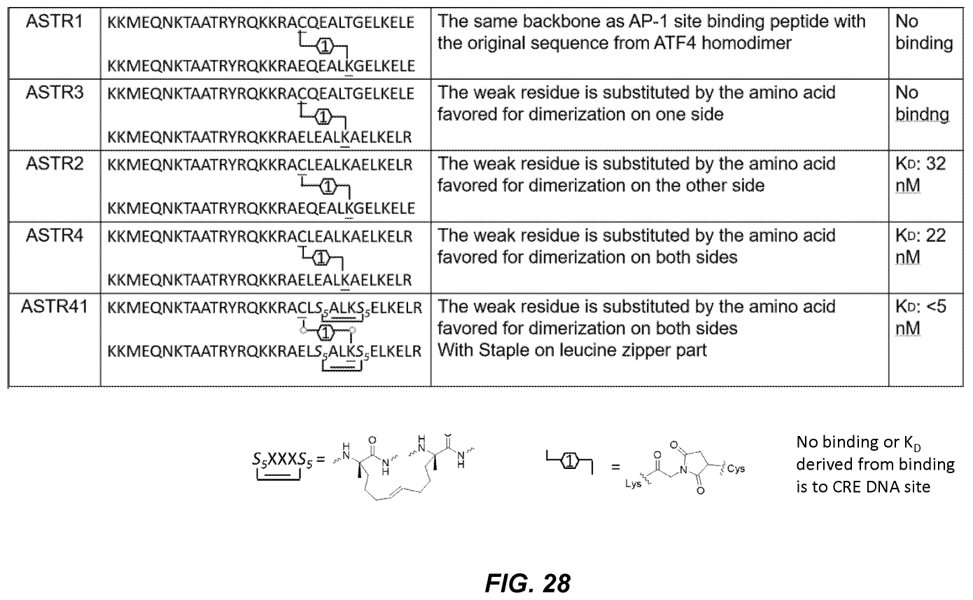

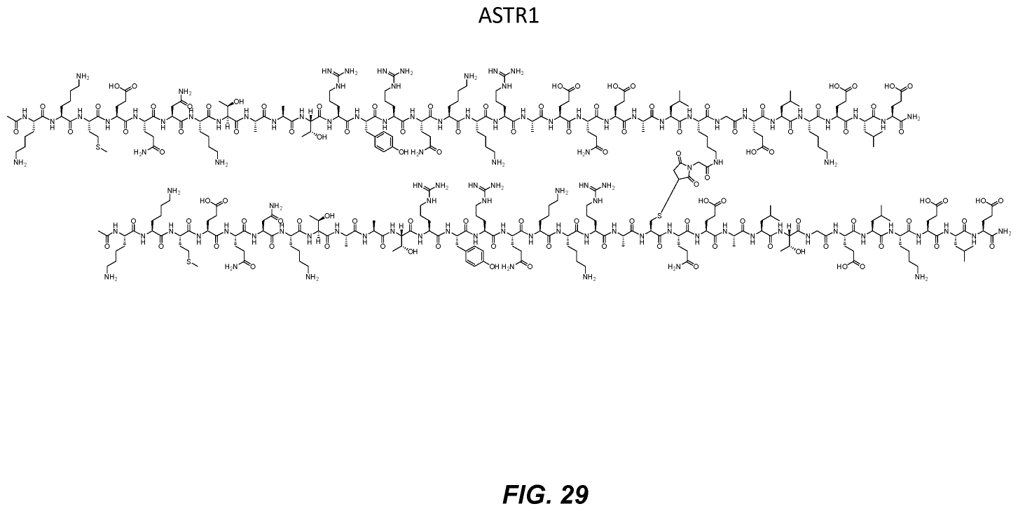

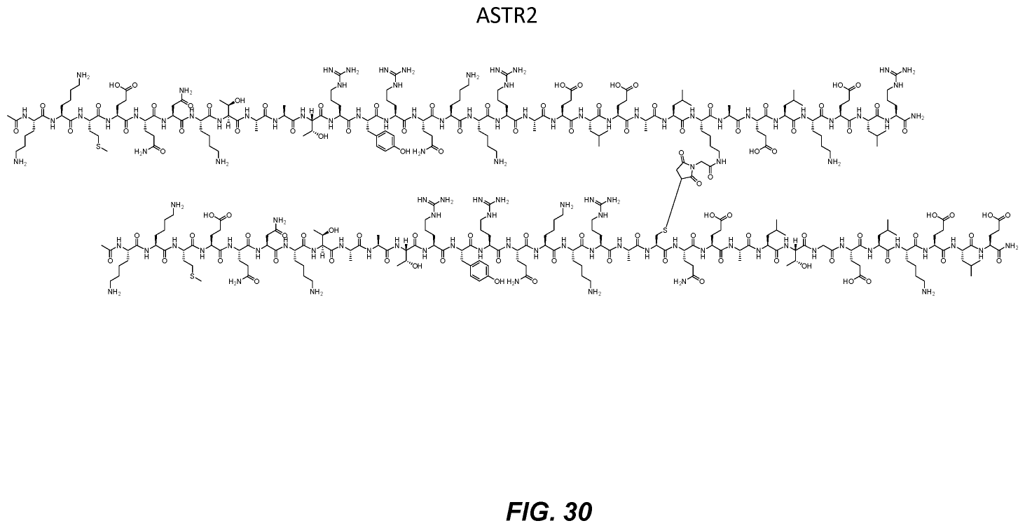

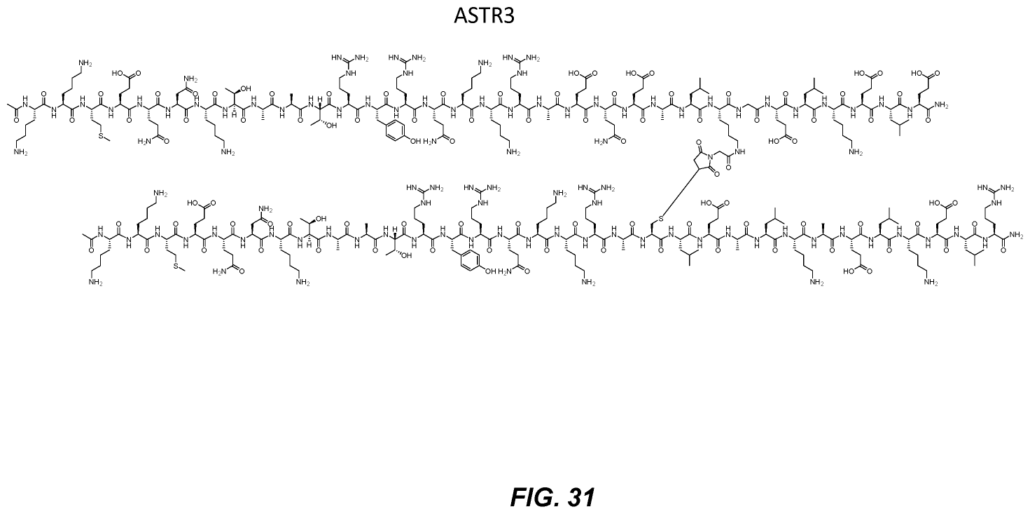

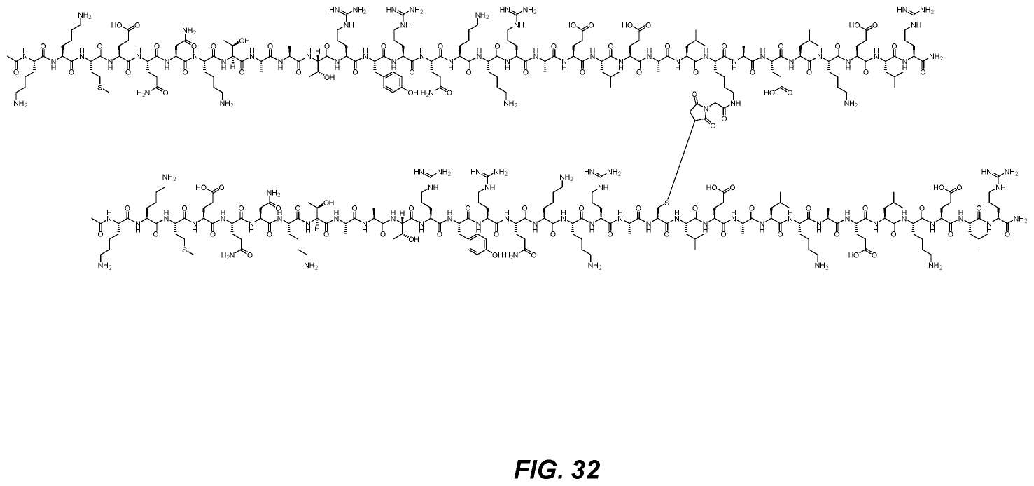

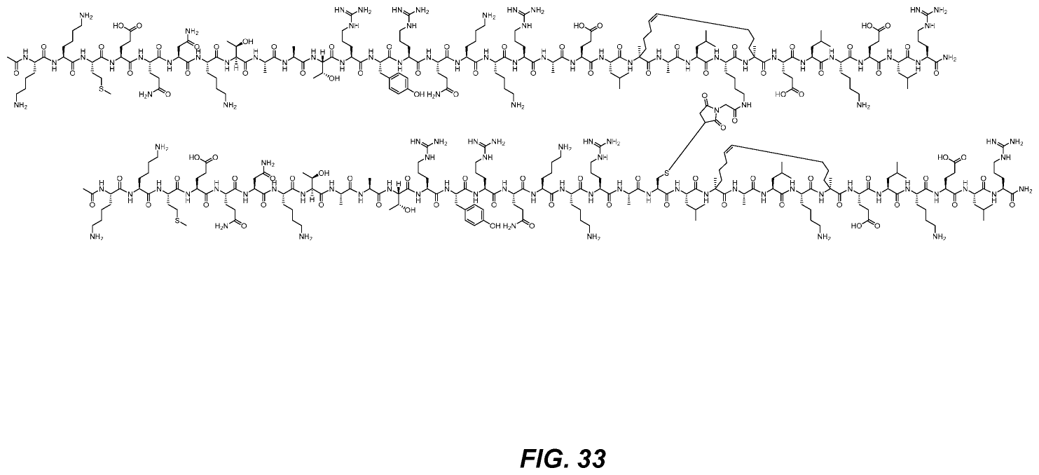

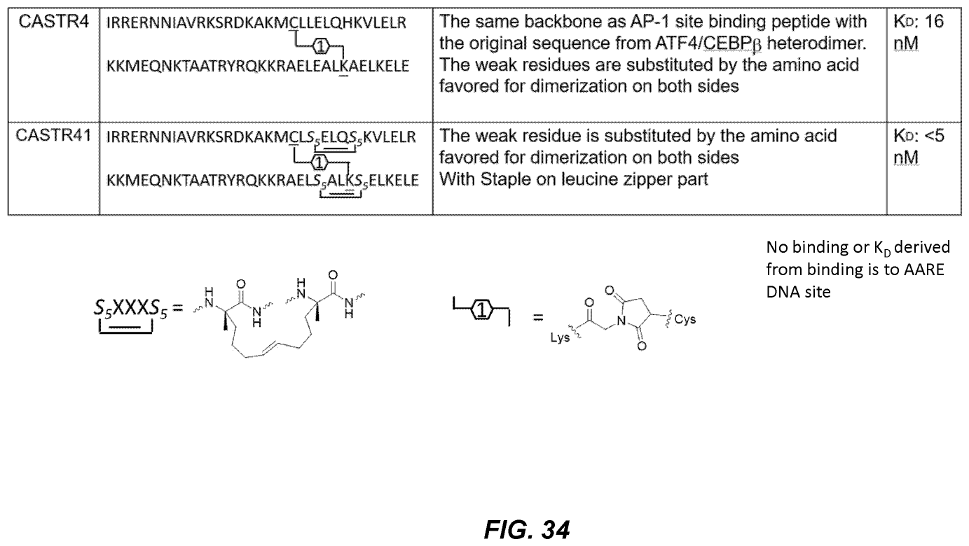

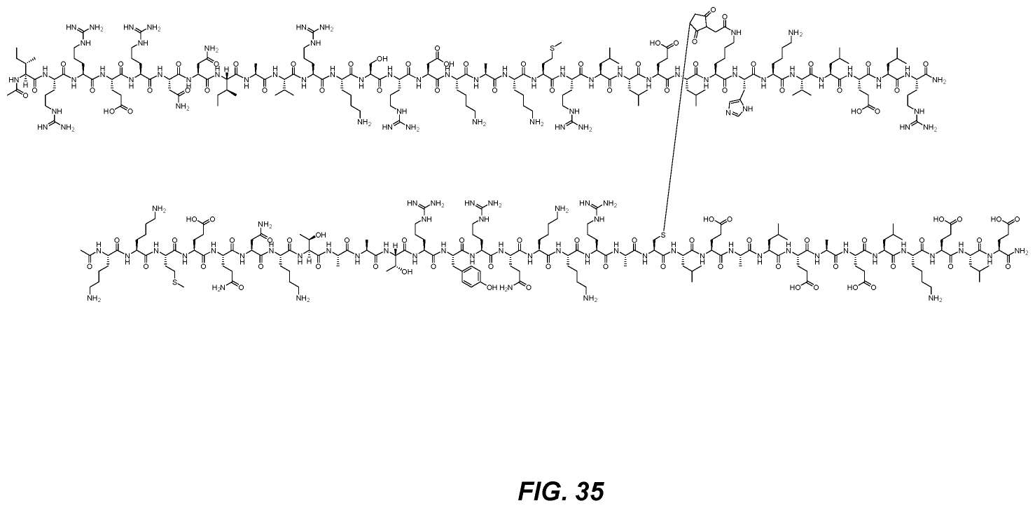

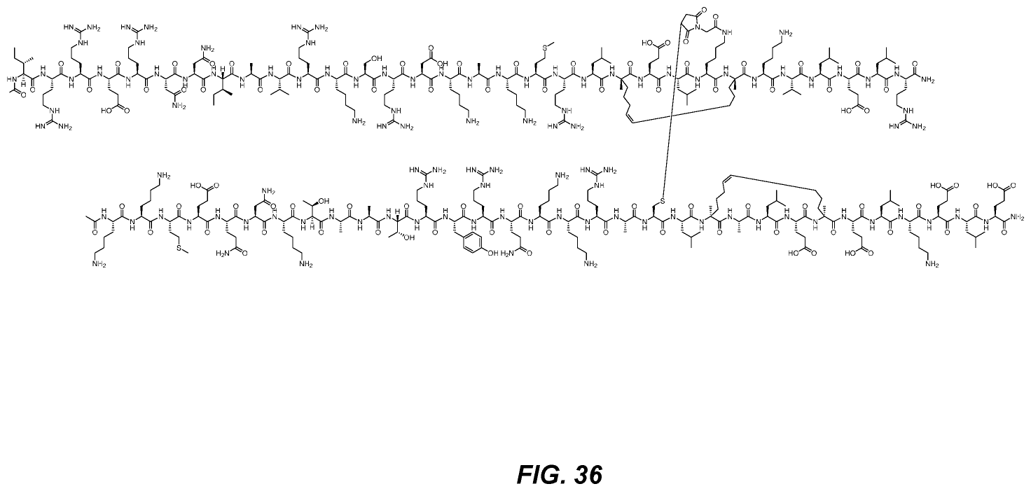

bZIP transcriptional repressors CASTR4 (shown in FIGS. 34 and 35), CASTR41 (shown in FIGS. 34 and 36), ASTR4 (shown in FIGS. 28 and 32), and ASTR41 (shown in FIGS. 28 and 33) were generated and tested. FIGS. 17-20 show results from various EMSA experiments, demonstrating DNA binding of CASTR4, CASTR41, ASTR4, and ASTR41.

### Example 8—Inhibition of HIF1α-Dependent Transcription and Oncogenic Phenotypes with XBP1-Derived Synthetic Transcriptional Repressors

The inventors hypothesized that convergent synthesis of stabilized, minimal mimetics of the bZIP DNA binding domain could enable potent and specific DNA binding of target sequences and competition with native TFs for those sites. Conceptually, this approach is supported by seminal work with the bZIP protein GCN4¹⁸, followed by efforts to engineer natural polypeptide mimetics of Zn-finger, bZIP and bHLH domain-based peptides and proteins¹⁹⁻²³. All of these approaches to mimic natural TF protein structures have relied on synthesis or expression of long, natural polypeptides, which suffer from low synthetic yields, reduced structural stability and concomitant losses in binding affinity. Moreover, natural polypeptides—especially the unstructured and highly charged DNA binding domains—have pharmacologic limitations due to low cell membrane penetration and susceptibility to proteolytic degradation in cells and tissues²⁴. Collectively, these liabilities have largely precluded the use of natural peptide chemical probes or therapeutics to target intracellular TF function.

Recent studies reported a general strategy to synthesize non-natural, stabilized TF mimetics derived from the DNA-binding domains of MAX and other bHLH TFs²⁵. These synthetic transcriptional repressors (STRs) incorporated several non-natural secondary and tertiary domain stabilizing elements, yielding molecules with DNA binding affinity and specificity equivalent to full-length TF proteins. Optimized STRs exhibited improved structural and pharmacologic stability relative to natural TF polypeptides, which correlated with the ability to penetrate cells intact and compete with native TF-DNA binding by MYC and MAX. The bHLH-derived STR architecture was shown to be modular within the bHLH family but is unlikely to be portable to others like the bZIP TFs due to the unique three-dimensional structure required for DNA binding. Certain aspects herein describe a strategy to create STRs that recapitulate bZIP DNA binding architecture to antagonize XBP1- and HIF1α-DNA binding and transactivation in vitro, in cells and in vivo.

### Example 9—XBP1s-Derived Synthetic Transcriptional Repressors Specifically Bind DNA and Penetrate Cells

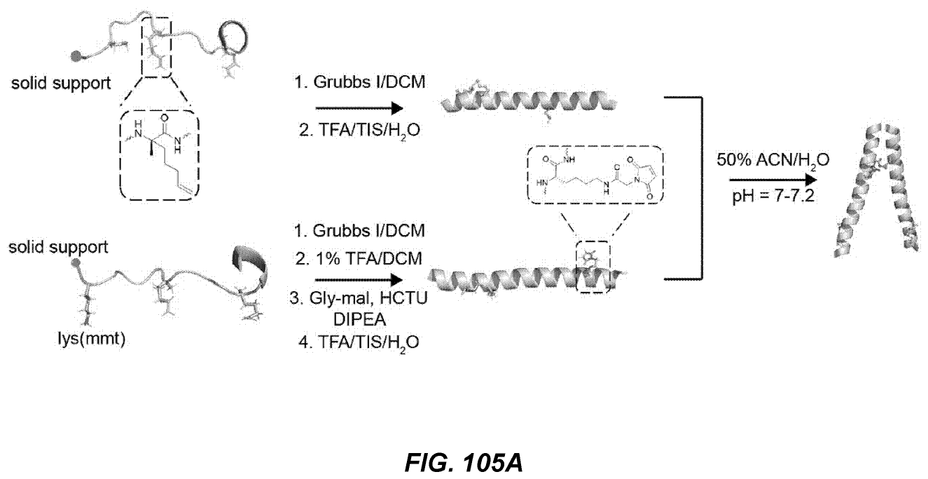

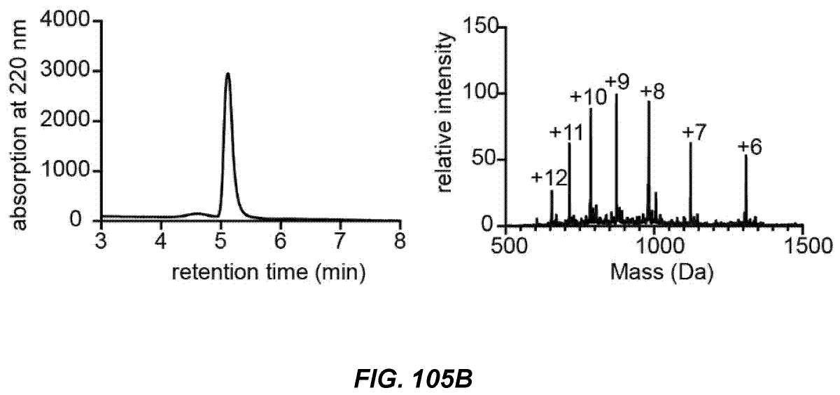

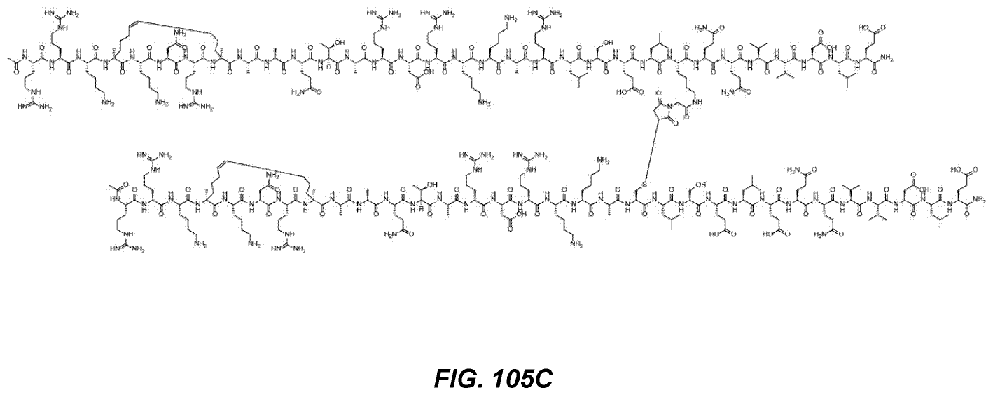

Based on the fact that the HRE motif 5′-ACGTG-3′ is embedded within the canonical UPRE motif (5′-TGACGTGG-3′), which is bound and regulated by XBP1s (FIG. 101A), the inventors reasoned that a molecule capable of mimicking the DNA-binding specificity of the XBP1s homodimer could serve as a dual-antagonist of both XBP1s and HIF1α-regulated target genes containing UPRE/HRE regulatory sequences (FIG. 101B). The inventors hypothesized that potent, specific and pharmacologically stable bZIP TF mimetics could be developed through the synthesis of stabilized proteomimetics containing four non-natural design elements, including: i) Individual peptides encompassing empirically-identified, minimal regions of the bZIP-domain of XBP1; ii) Identification of interhelix ligation positions, crosslinking moieties and suitable orthogonal chemistries to site-selectively dimerize each helix monomer into a DNA binding tertiary structure; iii) Incorporation of side-chain macrocycles²⁶ for helix nucleation and global structure stabilization; iv) Optimization of helix-helix interface contacts and solvent-exposed residues for structural and pharmacologic stabilization²⁷ (FIG. 101B). The inventors predicted that a suitable combination of these elements, but not necessarily each individually, would create TF mimetics that retain high affinity and specificity for target DNA sequences and compete with endogenous TFs for binding to target DNA. Using this blueprint and an iterative process of design, synthesis (Extended Data FIG. 1a) and biochemical testing, the inventors determined that potent and specific STRs could be developed from residues Arg75-Leu105 of XBP1s (FIG. 101C, FIG. 105A).

With this monomeric footprint the inventors found that ‘face-to-face’ ligation of orthogonally protected Cys and Lys residues with a glycylmaleimide linker could create a covalently linked bZIP domain mimic with proper alignment of each helix for DNA binding. Intriguingly, the inventors found that the natural hydrophobic contacts formed between each helix were not optimal when incorporated into a more compact STR mimetic, however a Met-to-Leu mutation at the helix-helix interface transformed a progenitor molecule with no stable binding to UPRE-containing DNA (STR1) into a molecule with potent DNA binding affinity (STR4; K_{d}=49 nM, FIG. 101E). Incorporation of side chain, bis-alkylated hydrocarbon macrocycles into specific positions of each DNA binding helix resulted in a lead molecule, STR22, with high affinity to the consensus UPRE motif (K_{d}=11 nM) and undetectable binding to a control AP1 motif containing oligonucleotide (FIGS. 101D-101E).

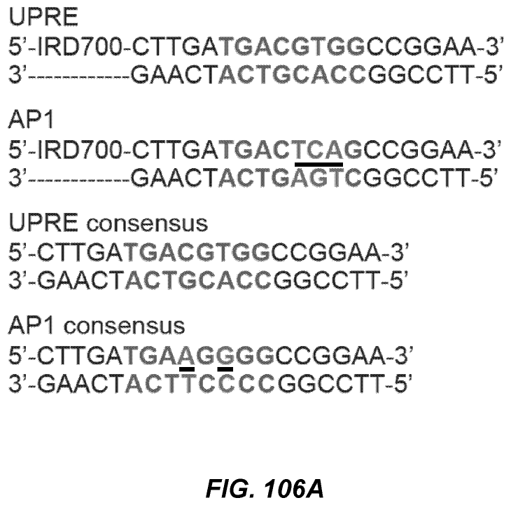

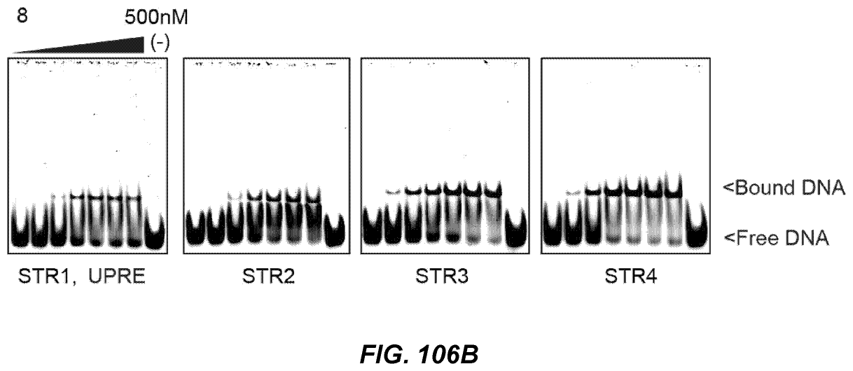

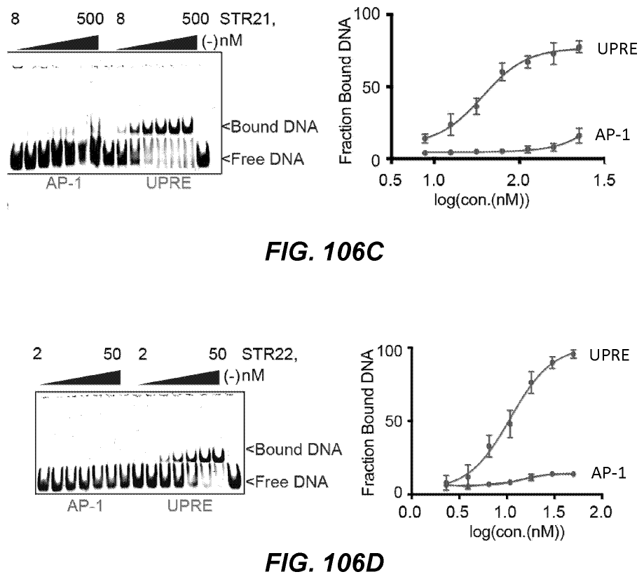

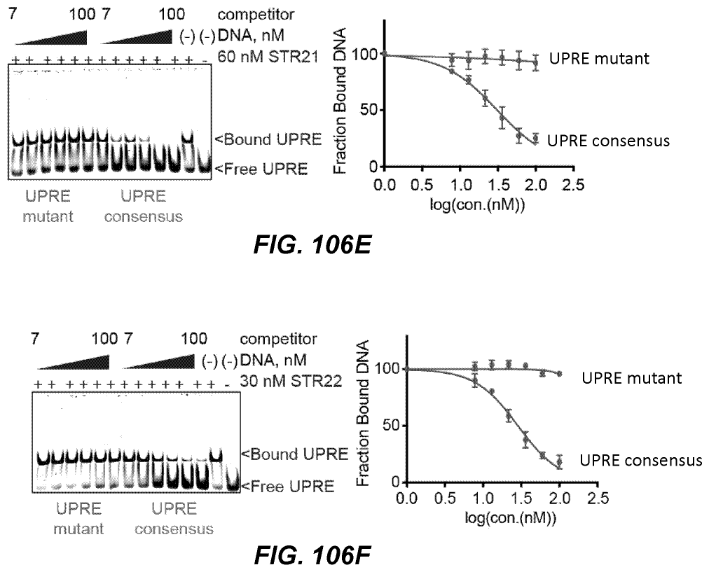

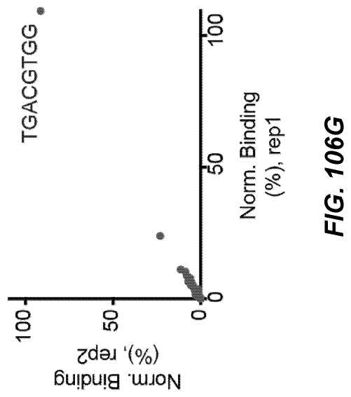

Competitive EMSA experiments demonstrated that STR22 binding was potently competed with excess unlabeled UPRE oligonucleotide, but not a mutant oligonucleotide, further confirming DNA binding specificity (FIGS. 106D-106F). In a quantitative analysis of binding to a pool of >30 natural TF operator motifs²⁵, STR22 specifically bound oligos containing a core UPRE/HRE sequence motif (FIG. 106G).

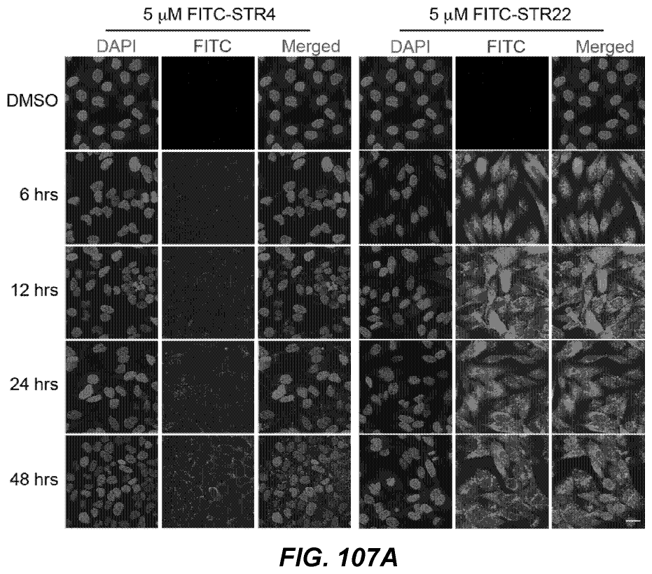

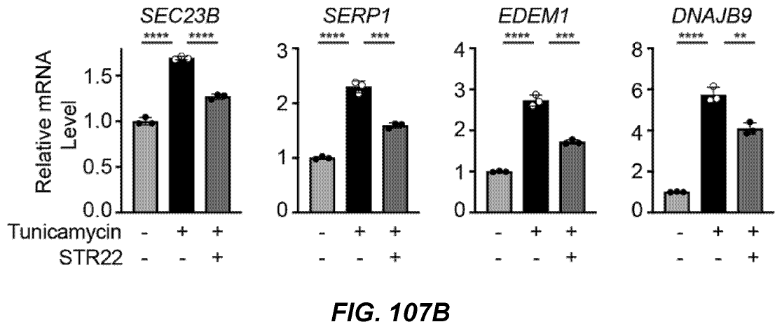

Previous mechanistic studies²⁸⁻³⁰ have demonstrated that a combination of structural stability, formal charge, protease stability and other features are correlated with the active uptake and cytosolic access of diverse stabilized peptides and miniproteins in cells³¹⁻³⁵, animals^{31,33-36} and humans³⁷. Consistent with this, the inventors found that the structurally distinct but analogous bHLH-derived STRs exhibited much higher protease stability and cellular uptake relative to peptides derived from natural DNA-binding domains (e.g., bHLH domain peptides from MAX)²⁵. Therefore, despite occupying a region of chemical space between traditional small molecules and large biologics, the inventors reasoned that stabilized bZIP-derived STRs should be capable of accessing the cytosol and nucleus of cells intact via active uptake mechanisms. Confocal imaging of HeLa cells treated for 12 hours with fluorosceinisothiocyanate (FITC)-labeled analogs of STR4 and STR22 confirmed that the synthetically stabilized molecule, STR22, was distributed throughout both cytosolic and nuclear compartments (FIG. 101F, FIG. 107A). By contrast, the analog without helical stabilization elements, STR4, showed a much lower level of intracellular fluorescence, despite being essentially identical in terms of molecular weight, charge and sequence. Fluorescence gel electrophoresis of the intracellular contents of cells treated with STR4 or STR22 also confirmed that full-length STR22 was present inside cells at significantly higher concentrations compared to STR4, and that these levels were stable over time (FIG. 101G), consistent with confocal imaging performed over longer incubation times (FIG. 107A). Together, these structure activity relationships confirmed that fully synthetic, minimal bZIP TF mimetics could be developed by layering several non-natural stabilization chemistries. Beyond generating molecules with TF-like affinity and specificity, the secondary and tertiary stabilizing elements in STR22 improve cellular uptake and stability.

### Example 10—Optimized XBP1-Derived STRs Block XBP1-Induced Gene Expression

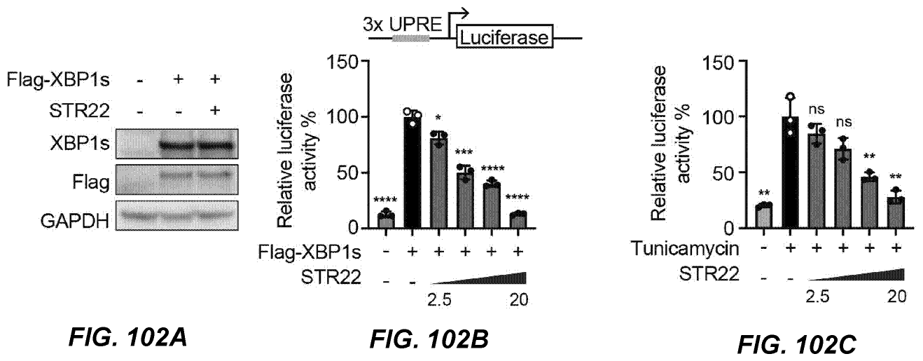

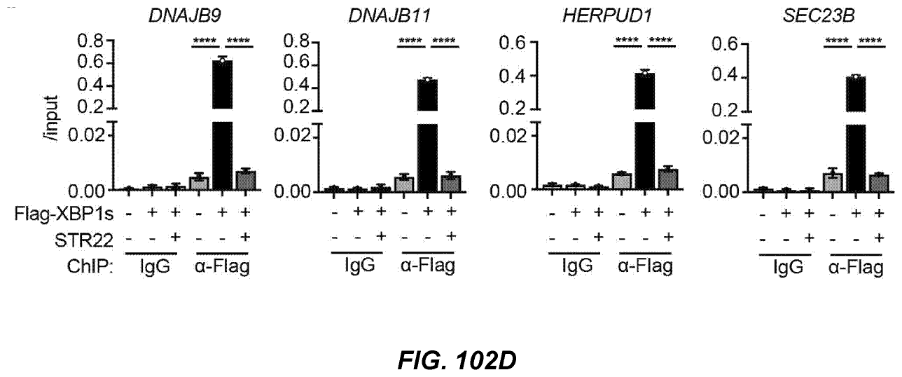

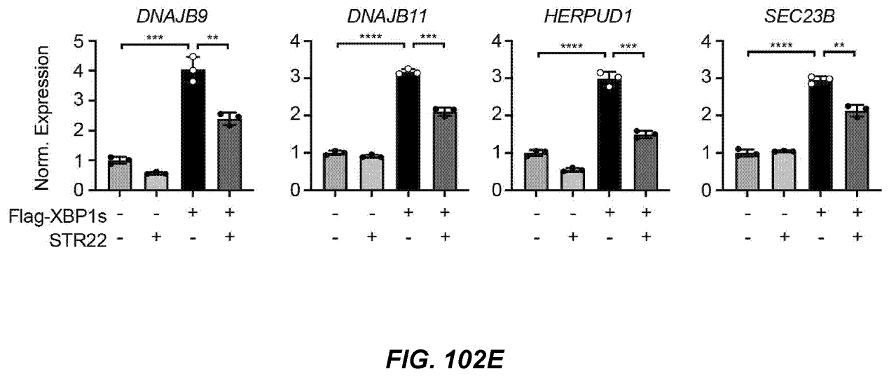

To directly test whether the DNA binding potency and cell-penetrant properties of STR22 enabled functional antagonism of XBP1-dependent transcription, the inventors first developed an XBP1s-inducible, UPRE-regulated firefly luciferase reporter system. HeLa cells co-transfected with FLAG-XBP1s showed significant induction of the UPRE-regulated luciferase signal. Treatment of these cells with STR22 did not affect FLAG-XBP1s protein levels but caused a dose-dependent inhibition of the XBP1s-induced reporter signal with an IC₅₀ of 7.4±3.5 μM (FIGS. 102A-102B). The inventors further validated that exogenously expressed FLAG-XBP1s directly binds to the promoters of canonical UPRE-motif regulated target genes, including DNAJB9, DNAJB11, HERPUD1 and SEC23B, as measured by chromatin immunoprecipitation and qPCR quantification (ChIP-qPCR); STR22 treatment significantly inhibited XBP1s binding to these endogenous target genes (FIG. 102C). FLAG-XBP1s expression induced mRNA transcripts of DNAJB9, DNAJB11, HERPUD1 and SEC23B, and STR22 treatment significantly inhibited this induction (FIG. 102D). Finally, treatment of cells with tunicamycin, a well-characterized activator of endogenous XBP1 splicing and downstream transcription, also increased expression of all target genes, and treatment with STR22 abrogated this induction in both luciferase and qPCR assays (FIG. 102C; FIG. 107B). Taken together, these data validate that STR22 directly inhibits XBP1s-binding to and transcriptional activation of canonical UPRE-regulated genes in cells.

### Example 11—STR22 Reprograms Global HIF1α Recruitment to and Activation of Hypoxia-Regulated Genes

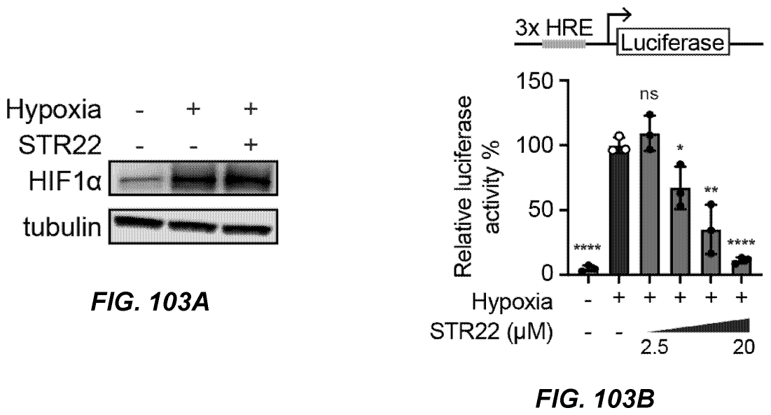

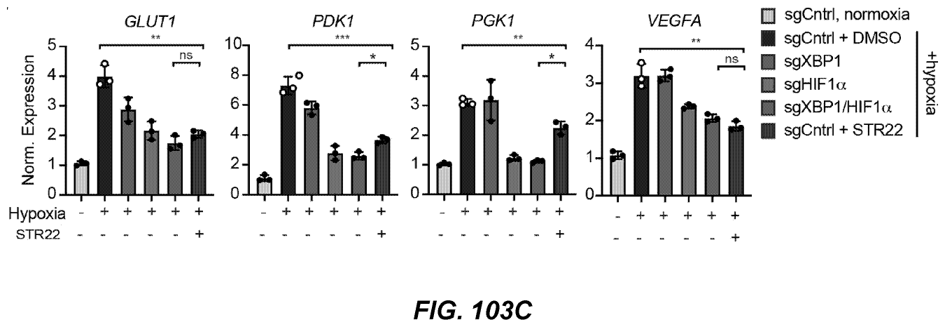

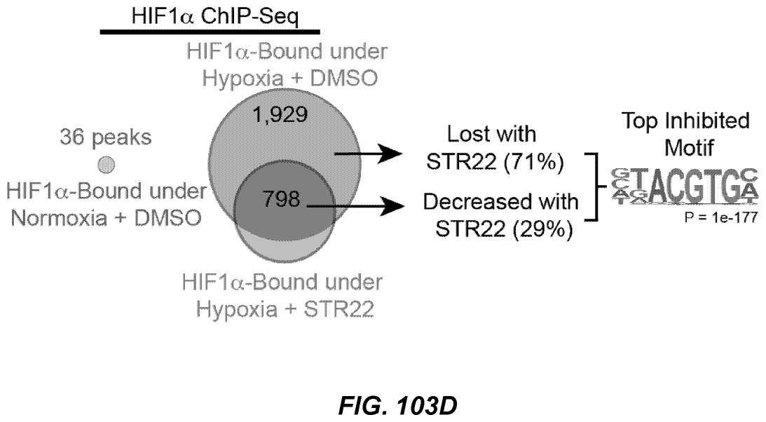

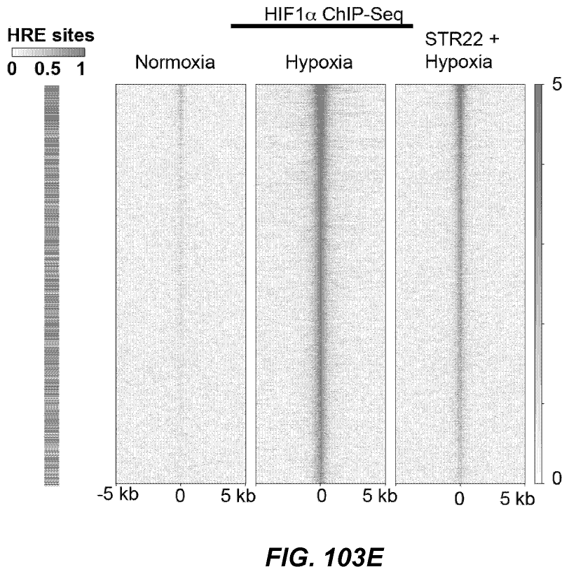

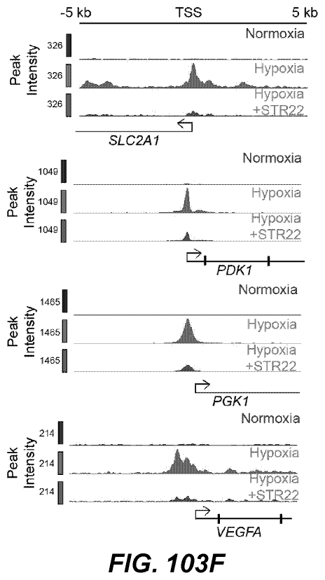

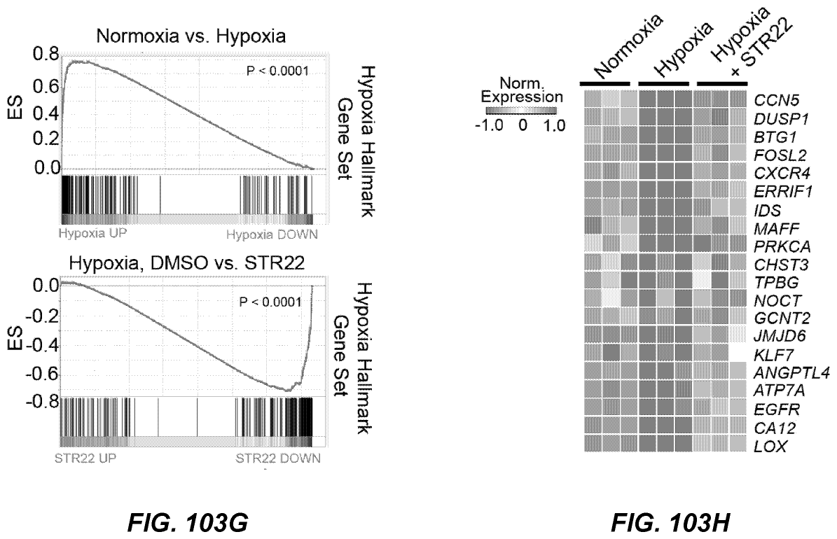

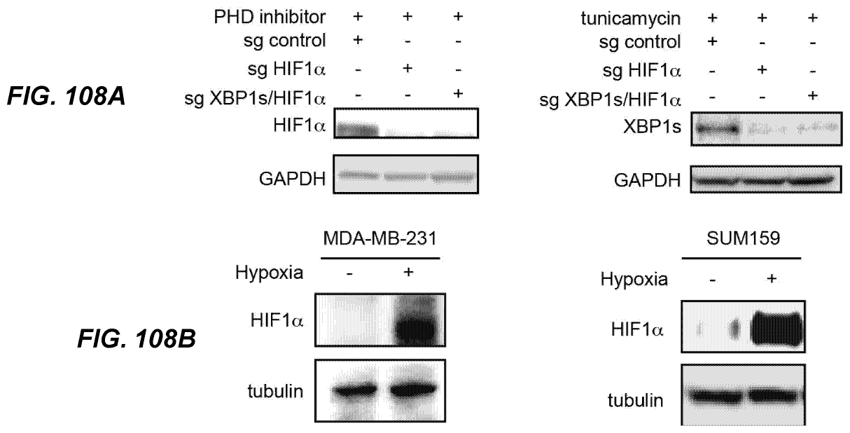

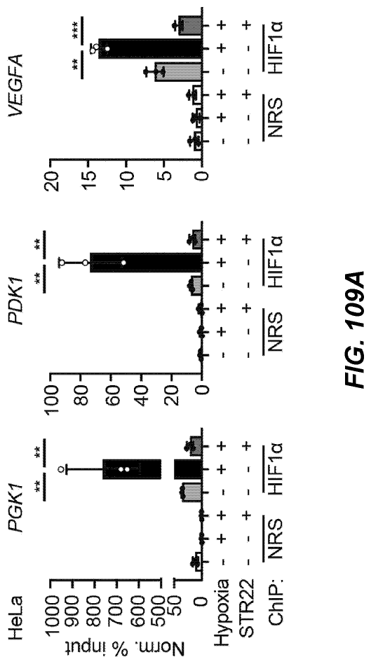

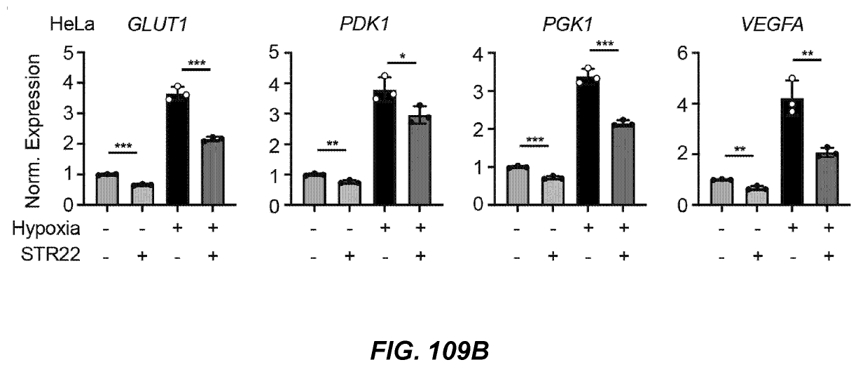

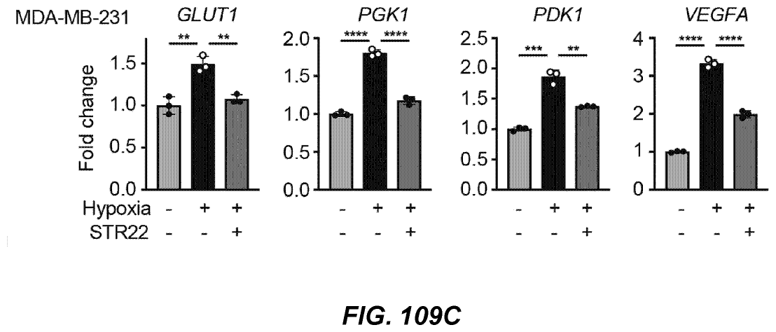

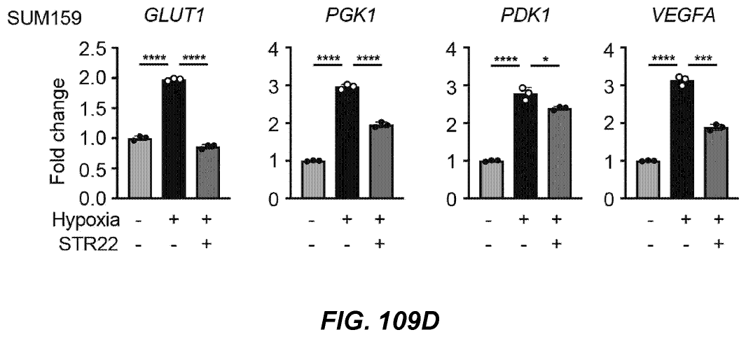

At the structural level STR22 to mimic the bZIP DBD architecture encoded by XBP1s in order to compete with endogenous TFs binding, the design strategy aimed to mimic XBP1s and bind target Due to the embedded overlap between HRE and UPRE DNA motifs¹⁵, the inventors next hypothesized that STR22 could antagonize HIF1α binding to and transcriptional activation of hypoxia-regulated genes in cells. Exposure of HeLa cells to hypoxia (1% 02, 6 hours) resulted in significant accumulation of HIF1α protein, and this induction was not affected by co-treatment with STR22 (FIG. 103A). Hypoxia treatment also drove the expression of HRE-regulated luciferase reporter gene expression, and STR22 treatment blocked this induction with potency equivalent to that observed in UPRE-reporter studies (FIG. 103B). ChIP-qPCR experiments demonstrated that endogenous HIF1α binding to canonical hypoxia-regulated genes PDK1, PGK1 and VEGFA is significantly increased in response to hypoxia. Under these conditions, STR22 abrogated HIF1α binding to and activation of these endogenous target genes (FIGS. 109A-109B). Using cells harboring Cas9-mediated knockout of HIF1α, XBP1 or both TFs, the inventors confirmed that HIF1α is the dominant inducer of these hypoxia-regulated target genes and that STR22 treatment mimics the HIF1α knockout (FIG. 103; FIG. 108A). Collectively, these data confirm that antagonism of hypoxia-induced gene expression is caused by direct competition by STR22, an XBP1 mimetic, with HIF1α binding to and regulation of HRE sites.

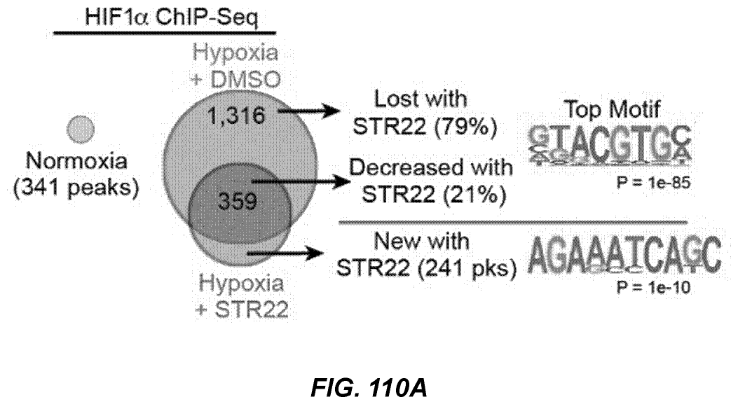

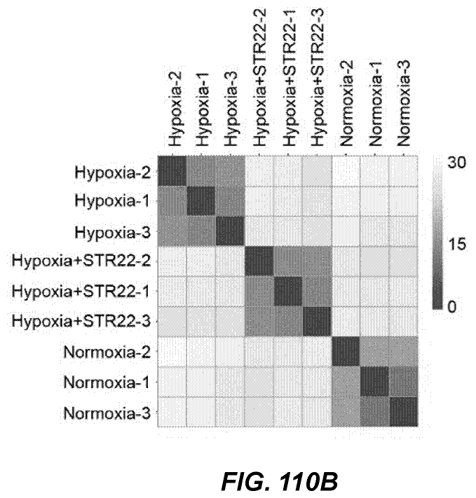

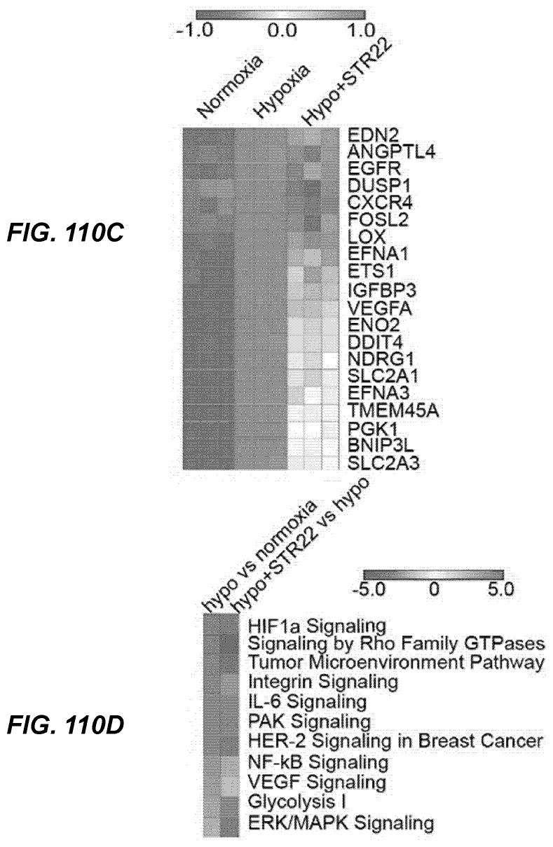

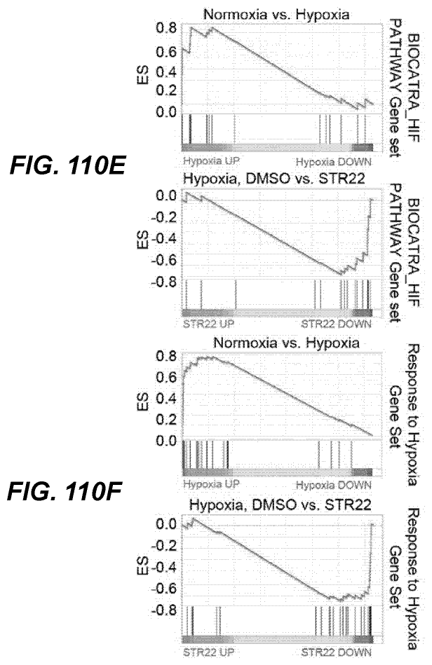

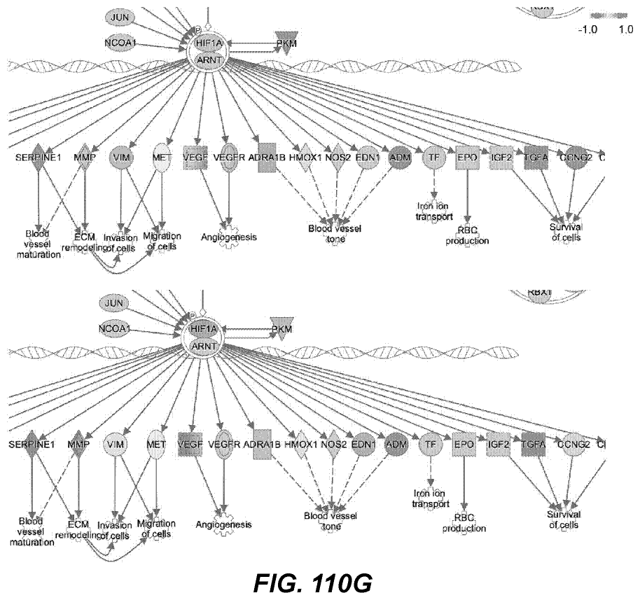

To study the effect of STR22 treatment on hypoxia-induced gene expression in more depth, the inventors performed ChIP-seq and RNA-seq profiling of HeLa cells under conditions of normoxia, hypoxia and hypoxia with STR22 treatment. Chromatin immunoprecipitation using an anti-HIF1α antibody identified 2,727 enriched peaks when comparing hypoxic versus normoxic treatment conditions, for which the canonical HRE motif 5′-ACGTG-3′ was the most enriched sequence (p-value=10⁻¹⁷⁷; FIGS. 103D-103E; FIG. 110A). Treatment of hypoxic cells with STR22 either completely or partially reduced HIF1α binding to 99.8% of these enriched peaks. Notably, approximately 10% of the peaks bound by HIF1α in cells treated with STR22 were new, non-HRE DNA sequences without a dominant consensus motif, suggesting that displacement HIF1α binding to HRE sites results in limited redistribution to lower affinity, non-functional sites in the genome. In agreement with targeted ChIP-qPCR studies (FIG. 109A) and literature precedent, HIF1α protein levels increased adjacent to the transcriptional start sites (TSSs) of PGK1, PDK1, GLUT1 and VEGFA in response to hypoxia, and STR22 treatment strongly inhibited this accumulation (FIG. 103F) at these canonical targets. mRNA sequencing and subsequent gene set enrichment analysis (GSEA) confirmed that STR22 treatment reverses the gene expression changes induced by hypoxia (FIG. 103G; FIGS. 110C-110F). For example, a hypoxia-responsive hallmark gene set from the Molecular Signatures Database was significantly enriched among the most upregulated genes of hypoxic vs. normoxic cells (FIG. 103G). These same genes were enriched as the most downregulated genes when comparing expression profiles of STR22 treated vs. DMSO treated hypoxic cells (FIG. 103G). Leading edge and bioinformatics analyses of these mRNA-seq profiles further validated STR22-mediated downregulation of hypoxia-induced genes throughout the HIF1α-signaling pathway (FIG. 103H; FIG. 109G), including angiogenic growth factors, anabolic genes in metabolism, and stress resistance^{38,39}. Taken together, these data validate STR22's global antagonistic effect on HIF1α-binding to and activation of HRE-regulated target gene expression.

### Example 12—Str22 Inhibits Aggressive TNBC Phenotypes In Vitro and In Vivo

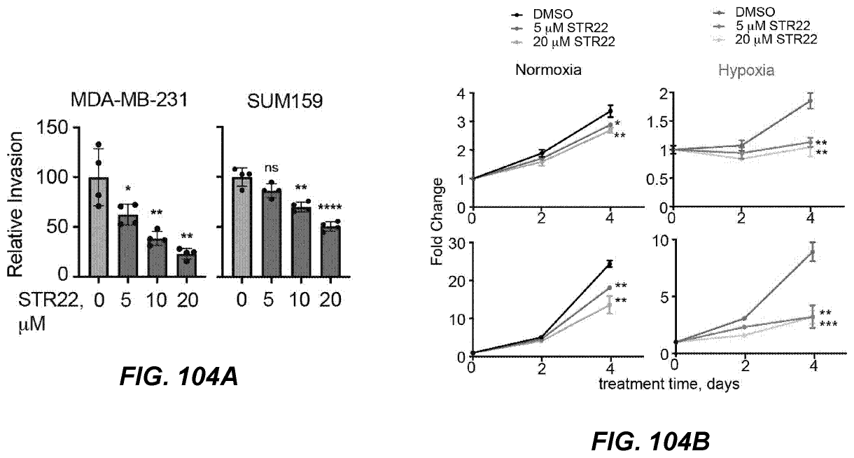

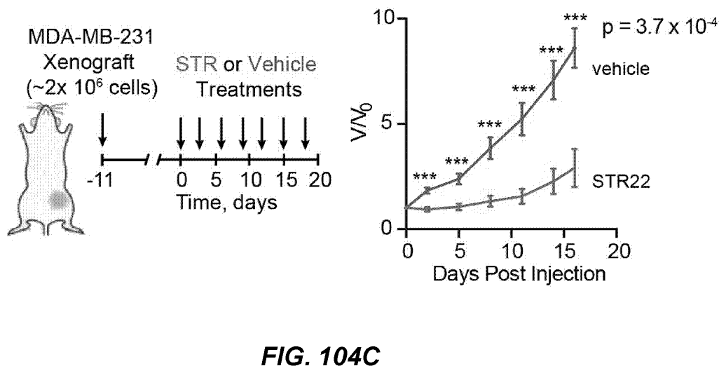

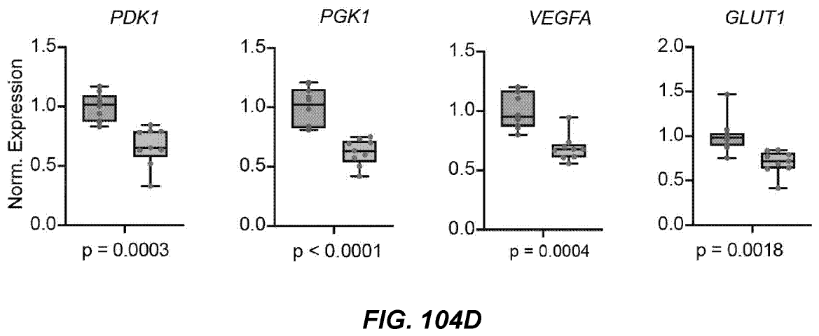

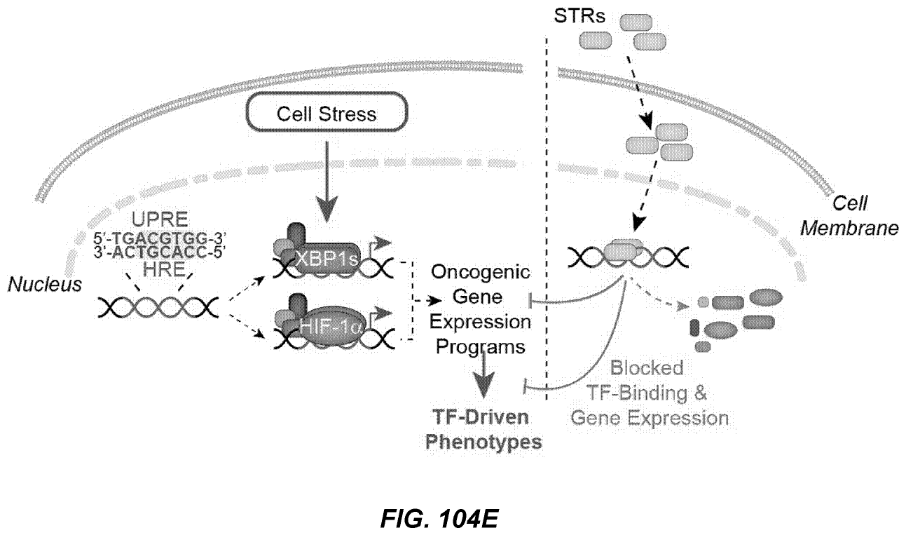

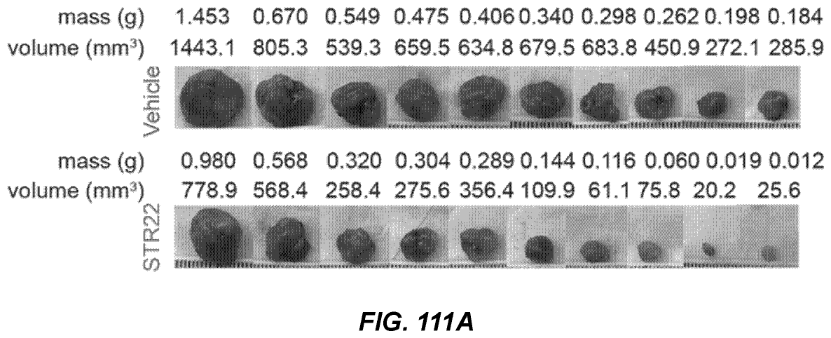

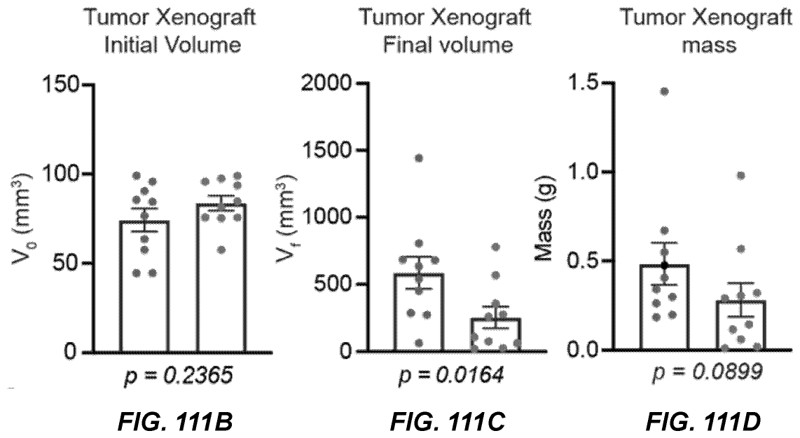

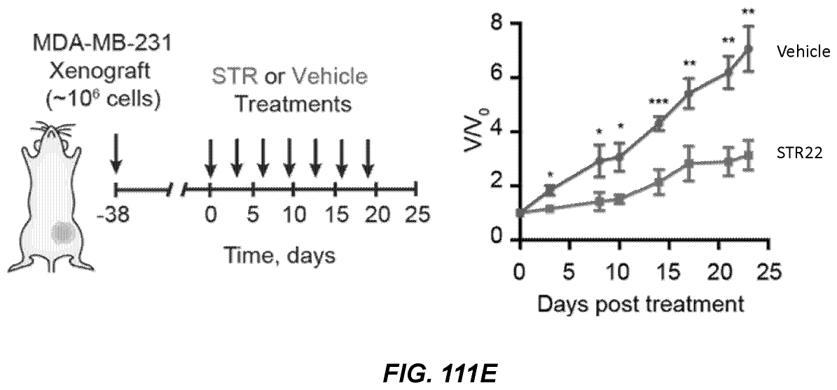

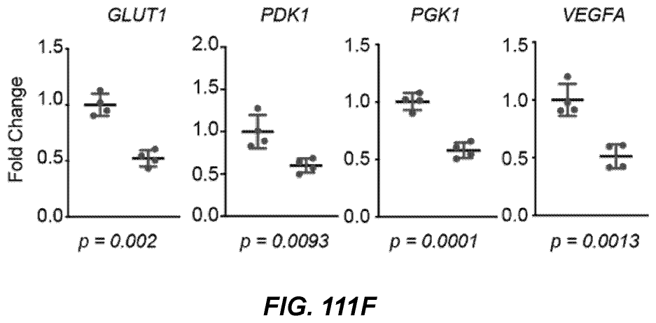

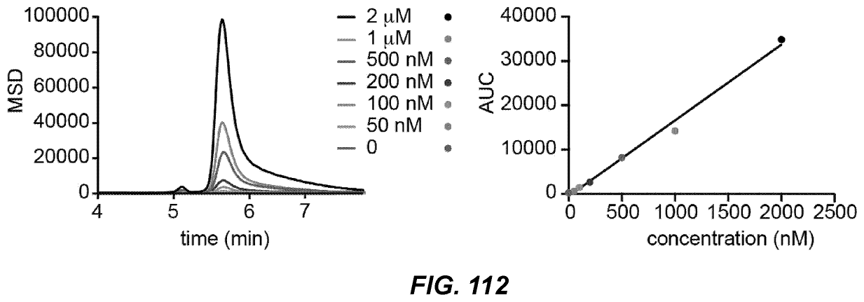

Increased abundance and activation of XBP1s and HIF1α are implicated in triple negative breast cancers and HIF1α specifically has been shown to correlate with the size^{16,17} and growth of human TNBC xenografts^{17,40}. Moreover, hypoxia-induced gene signatures have also been associated with poor disease outcomes in TNBC patients^{41,42}. Given these associations, the inventors sought to determine how STR22 antagonism of HIF1α-dependent signaling would affect TNBC cell phenotypes in cell culture and in vivo. Hypoxic treatment of model TNBC cell lines, MDA-MB-231 and SUM159, led to significant induction of HIF1α protein (FIG. 108B) and upregulation HRE-regulated genes; co-treatment with STR22 inhibited this induction (FIGS. 109C-109D). STR22 treatment inhibited cell invasion of MDA-MB-231 and SUM159 under hypoxic conditions, which is consistent with established associations between invasive phenotypes and hypoxic tumor microenvironments (FIG. 104A). Intriguingly, STR22 treatment had little effect on the proliferation of MDA-MB-231 and SUM159 under normoxic conditions, while matched treatments under hypoxic conditions significantly slowed cell growth (FIG. 104B). These data suggest that STR22 inhibition of HIF1α-mediated protective mechanisms under conditions of cell stress can impact tumor cell growth. Therefore, the inventors sought to determine whether STR22 treatment would affect the growth of TNBC cells in the in vivo context of tumor xenografts. To do so, the inventors generated MDA-MB-231 xenografts in the mammary fat pads of nude mice; after tumors reached 100 mm³—a size previously shown capable of creating hypoxic microenvironments⁴³—matched cohorts (n=10) were treated once every three days with intratumoral injections of STR22 or saline vehicle alone (FIG. 104C). Tumor volume was significantly inhibited in mice treated with STR22 at all timepoints (FIG. 104C and FIGS. 111A-111D). After approximately three weeks of treatment, mice in both groups were sacrificed 24 hours after the last injection and tumors were excised, weighed and processed for mRNA extraction. qPCR quantification of four canonical HIF1α-dependent target genes confirmed inhibition of hypoxia-induced gene expression in animals by STR22 treatment (FIG. 104D). A separate study using fewer MDA-MB-231 cells to generate xenografts replicated these findings (FIGS. 111E-11F). Taken together, these data confirm that STR22 exerts anti-proliferative effects in TNBC tumors.

### Example 13—Methods and Materials for Certain Aspects

Cell Culture. HeLa, MDA-MB-231 and SUM159 cells were purchased from ATCC and cultured in DMEM supplemented with 10% FBS and 1% penicillin/streptomycin. All cell culture was performed under 37° C. with 5% CO₂ unless otherwise indicated.

STR Synthesis and Purification. A Symphony X automated peptide synthesizer was used to prepare linear peptides on Rink amide MBHA resin. Fmoc-based solid phase chemistry, ring closing metathesis, and N-terminal modifications were carried out as previously described⁴⁵. Lysine residues bearing monomethoxy trityl (Mmt) side chain protecting groups were incorporated at cross-linking positions of one branch. On-resin Mmt deprotection was carried out for 5×2 min consecutive cycles of 1% TFA/DCM solution mixed by N₂ bubbling. Deprotected lysine residues were functionalized with maleimide by a 2 hr treatment with a 0.1 M solution of 2-(2,5-dioxo-2,5-dihydro-1H-pyrrol-1-yl) acetic acid (Mal-Gly-OH) (5 eq), HCTU (4.8 eq), and DIPEA (10 eq.) in DMF. Crude peptides cleaved from resin were purified on a Waters preparatory HPLC system using an Xbridge Prep C18 5 μm OBN (19.5×150 mm) column; solvent A (0.1% TFA in H₂O); solvent B (MeOH); and a 10-min method with the following gradient (flowrate=20 mL/min): 35% B over 1 min; 35-85% B over 7 min; 95% B over 1 min; 35% B over 1 min. STR monomer ligation was performed in 50% ACN/H₂O as follows: a purified peptide sequence bearing a maleimide (0.5 mL, 0.5 mM) and another purified peptide sequence with a free thiol (0.5 mL, 0.5 mM) were combined in a microcentrifuge tube and then pH-adjusted with N-methylmorphline to 6.8-7.2 based on pH test paper and then incubated for 1 hr at room temperature. The reaction mixture was purified using the same HPLC method as for individual monomers. STR purity was confirmed by LC-MS using an Agilent system equipped with a Phenomonex C18, 5 μm (5.0×50 mm) column; solvent A (95:5:0.1 H₂O/ACN/TFA) and solvent B (95:5:0.1 ACN/H₂O/TFA); 0.5 ml min⁻¹ flowrate, 0-2 min (0% B), 2-16 min (0-75% B), 16.5-18.5 min (100% B), 19 min (0% B). STR concentrations were quantified by mass and compounds were stored as lyophilized powder or in DMSO stocks.

Electrophoretic Mobility Shift Assays (EMSAs): For direct DNA binding experiments, STRs were serially diluted at 10× concentration in water and then 1 μL of STR solution was added to 9 μL of 5 nM IRD700-labeled DNA probe bearing either a UPRE or AP-1 motif in a final 1×binding buffer (20 mM HEPES pH 8.0, 150 mM NaCl, 5% glycerol, 1 mM EDTA, 2 mM MgCl₂, 0.5 mg/mL BSA, 1 mM DTT, 0.05% NP-40). Samples were incubated for 1 hr at RT. 5 μL of each reaction was loaded on an 8% acrylamide, 0.5×TBE gel equilibrated to 4° C. Samples were resolved for 60 min at 110 V and 4° C. with 0.5×TBE+1 mM MgCl₂ running buffer. Gels were pre-run at 110 V for 15 min prior to sample loading. For competition experiments, 30 or 60 nM STRs and 5 nM labeled UPRE probe were incubated with 0, 7.8, 13, 21.6, 36, 60 or 100 nM unlabeled competitor oligo for 1 hr at RT. Gels were imaged using a Li—COR Odyssey. ImageJ was used to quantify band intensity and the fraction of bound DNA was calculated by dividing the band intensity of bound DNA by the band intensity of free DNA from the vehicle treated lane. A four-parameter dose-response curve fit to a plot of normalized fraction bound DNA vs. log STR concentration yielded an the apparent K_{D}. Mobility shift data were excluded from analysis when higher order binding species were observed.

Quantitative, Multiplexed EMSA (qEMSA): A set of 33 unique DNA motif targets were designed and flanked by 12 unique barcoded forward primers and a single universal reverse primer (Extended Data Table 1). The DNA targets were pooled in sets of 12 comprised of the canonical UPRE target sequence 1 and 11 barcoded competitor motifs to a final concentration of 4 nM (2×) each target in 1×binding buffer. STRs were prepared at 2× concentration in 1×binding buffer (10 nM for STR1, STR4, STR21 and STR22). 10 μL of pooled DNA targets and 10 μL of STR were mixed and incubated for 15 min at RT followed by 15 min at 4° C. 5 L of each sample was loaded into a 10% acrylamide 0.5×TBE native gel equilibrated to 4° C. Electrophoresis was carried out at 150V for 120 min at 4° C. The gel was then stained using EtBr (0.5 g/mL in 0.5×TBE) for 30 min at RT and destained in DI water for 10 min. The gel was visualized using a Spectroline model TE-132S transilluminator and the shifted DNA band representing the bound targets was excised. The excised DNA was extracted using a QIAEX II gel extraction kit (Qiagen) following the manufacturer's protocol for acrylamide gels. The purified DNA was analyzed by quantitative PCR usinga SYBR green master mix (Applied Biosystems) on a Lightcycler 480 II (Roche). Relative enrichment to the E-box target sequence was determined as the change between cycle threshold values (Ct) of E-box target and TF motif (100*2− [symbol] Ct), and independent replicates were plotted against each other.

Fluorescence Microscopy and Quantitative Analysis. HeLa cells were seeded in 12-well chamber slides with 2,500 cells per well (Ibidi, 81201). Once cells reached 40-50% confluency, they were treated with either DMSO, or 5 μM FITC-labeled STR for indicated durations. For shorter treatment times (<24 hrs), cells were grown to 70-80% confluency before start of treatment.

At the end of treatment, cells were washed with phosphate buffered saline (PBS) four times, fixed with 4% formaldehyde in PBS at room temperature for 10 mins, and then washed twice with PBS. Nuclei were labeled with DAPI (Thermo, D1306) in PBS at room temperature for 3 mins. Rubber gaskets and chambers were removed, and slides were dried in the dark at room temperature. When dry, cover glass (Fisher, #12-545 M) was mounted with 50 μL anti-fade mounting solution (Invitrogen, P36961), and sealed with nail polish. A Leica SP8 Laser Scanning Confocal with HyD detectors was used to image a single focal plane to accurately detect the DAPI and FITC signal. Identical microscope acquisition parameters were set and used within experiments to control for exposure. Post-acquisition processing was performed using ImageJ software⁴⁶. Loss-less TIFF files were employed to quantify fluorescence intensity.

PAGE Gel Analysis of STR Uptake. Approximately 1×10⁵ HeLa cells were seeded in each well of a 12-well plate. Cells were treated with 5 μM of either FITC-STR4 or FITC-STR22 for 6, 12, 24 and 48 hrs. After the indicated treatment time, media was aspirated, cells were washed with PBS (2×1 mL) and treated with 0.25% trypsin (0.2 mL) for 5 min at 37° C. The trypsin was quenched with the addition of 1 mL of media and the detached cells were transferred to a microcentrifuge tube and centrifuged at 2000 g for 1.5 min. The media was aspirated, 20 μL of RIPA buffer (50 mM Tris, pH 7.4, 150 mM NaCl, 0.25% deoxycholate, 1% NP-40, 1 mM EDTA) was added and cells were incubated for 10 min on ice. After lysis, 6.6 μL of 4×SDS loading buffer was added, samples were heated to 95° C. for 5 minutes, cooled to RT and resolved on a 4-20% Tris-glycine SDS-PAGE gel with a fluorescent filter to image FITC-labeled molecules.

Luciferase Assays. Approximately 2×10⁴ HeLa cells were seeded in the each well of a 96-well plate. Cells were co-transfected with 3×UPRE-luc (Addgene, 101788) and XBP1s overexpression construct or control vector using Lipofectamine 3000 (Invitrogen) for 4 hrs, which was then followed by treatment with STR22 for 24 hrs. Cells were then lysed in diluted cell culture lysis 5× buffer (Promega). 1× luciferase reagents (1 mM D-luciferin, 3 mM ATP, 15 mM MgSO₄, 30 mM HEPES pH=7.8) were then added and the mixtures were read for luminescence. For hypoxia response experiments, cells were transfected with 5×HRE-luc (PGK1-derived HRE promoter, Addgene, 128095) vector using Lipofectamine 3000 (Invitrogen) for 4 hrs and then treated with STR22 under 1% 02 (hypoxia) for 24 hrs. Luminescence was read following the aforementioned protocol.

Chromatin Immunoprecipitation (ChIP): For XBP1s-ChIP experiments, 3×10⁶ HeLa cells were transfected with 1 μg of either the control or Flag-XBP1s vector for 4 hrs and then followed by 20 μM STR22. For hypoxia response experiments, 10×10⁶ HeLa cells were treated with 20 μM STR22 for 24 hrs, followed by an additional 6 hrs under 1% 02. At the end of all treatments, cells were crosslinked with 1% formaldehyde for 10 min at 37° C. and then quenched with 125 mM glycine for 1 min. Cells were sheared in buffer containing: 0.1% SDS, 50 mM Tris-HCl (pH 7.6), 1 mM EDTA (pH8.0), 0.002% Triton X-100, supplemented with PMSF (Roche, 10837091001) and protease inhibitor (Roche, 11836170001). Lysates were sonicated with a Bioruptor for a total of 4 cycles for ChIP-qPCR, or 40 cycles for ChIP-sequencing (30 sec on/30 sec off each cycle). Sonicated chromatin was incubated with antibodies overnight at 4° C. Antibodies used: HIF1α (Novis Biologicals, NB100-479), normal rabbit serum (Jackson Immunoresearch, 011-000-001), Flag (Sigma, F1804-200UG), mouse IgG conjugated with Dynabeads Protein A (Life Technologies, 10001D)+G (Invitrogen, 10003D). Beads were washed twice for 5 mins each, first with RIPA buffer (10 mM Tris-HCl pH7.6, 1 mM EDTA, 0.1% SDS, 0.1% NaDOC, 1% Triton X-100), then RIPA buffer supplemented with 0.3M NaCl, LiCl buffer (0.21 M LiCl, 0.5% NP-40, 0.5% NaDOC) and finally TE buffer plus 0.2% Triton X-100. Next, beads were washed once with TE buffer for 5 mins. Beads were eluted in buffer containing 0.003% SDS, 10 mM Tris-HCl (pH8.0) and 1 mM EDTA (pH 8.0), 0.1 mg/ml Proteinase K (Fisher Scientific, 25-530-049) for 4 hrs at 65° C. ChIP-DNA was purified by AMPure XP (Beckman Coulter, a63881). DNA was then either subjected for sequencing or real-time PCR analysis. Primers used:

control:
forward:
5′-TGAGGGTTCATCAAGCTGGTGTCT-3′,
reverse:
5′-TTGGAGAGGGCAGTGCTTAACTCA-3′.
DNAJB9:
forward:
5′-AGCCCTAGCAGCAACAACAG-3′,
reverse:
5′-CACGAAACGCTTCCCCATTG-3′,
DNAJB11:
forward:
5′-CACAATTGGTTGGTGCTGGG-3′,
reverse:
5′-GGCACCTGAGGTACACGAAA-3′;
HERPUD1:
forward:
5′-GCGAGTCATTTCACCCTCCA-3′,
reverse:
5′-AACCTCATTTTGCAGCACGG-3′;
PDK1:
forward:
5′-CCGGTGACAGCCGATCC-3′,
reverse:
5′-AGAAGCCACAGCCAGCCAGTACG-3′;
PGK1:
forward:
5′-GGGAAGGTTCCTTGCGGTTC-3′,
reverse:
5′-GTCCGTCTGCGAGGGTACTA-3′;
VEGFA:
forward:
5′-TCTTCGAGAGTGAGGACGTGTGT-3′,
reverse:
5′-AAGGCGGAGAGCCGGAC-3′.

Real-Time PCR. mRNA was extracted using RNeasy plus Mini Kit (Qiagen). 1.5 μg mRNA sample was reverse transcribed into cDNA using a reverse transcription kit (Invitrogen A48570) and then subjected to SYBR-green based real-time PCR analysis (Invitrogen A25741). Primers used:

RPL13A:
forward:
5′-CTCAAGGTGTTTGACGGCATCC-3′,
reverse:
5′-TACTTCCAGCCAACCTCGTGAG-3′;
DNAJB9:
forward:
5′-ACACTGGATCCAAGAAGCGT-3′,
reverse:
5′-TTGAGTGACAGTCCTGCAGT-3′;
DNAJB11:
forward:
5′-ACGCTGGAAGTAGAAATAGAGCC-3′,
reverse:
5′-TCGGAACCGTAAATCTCCAGGC-3′;
DNAJC3:
forward:
5′-GGAGAGGATTTGCCACTGCTTTT-3′,
reverse:
5′-CTCTGCTCGATCTTTCAGGGCA-3′;
EDEM1:
forward:
5′-GAGGTGAGTTCTTCCTGCCT-3′,
reverse:
5′-AAACACGTTTCCTGACAGCC-3′;
HERPUD1:
forward:
5′-CCAATGTCTCAGGGACTTGCTTC-3′,
reverse:
5′-CGATTAGAACCAGCAGGCTCCT-3′;
HRD1:
forward:
5′-CCAACATCTCCTGGCTCTTTCAC-3′,
reverse:
5′-GTCAGGATGCTGTGATAGGCGT-3′;
SEC23B:
forward:
5′-CCGAATTGATGCCCCAGTTT-3′,
reverse:
5′-TCATGAACCTGCACCATCCT-3′;
SERP1:
forward:
5′-GTAAGGGAGCAGAGTGGTTAAG-3′,
reverse:
5′-CTGGGTGAGAAAGGCAAGTAA-3′;
GLUT1:
forward:
5′-TTGCAGGCTTCTCCAACTGGAC-3′,
reverse:
5′-CAGAACCAGGAGCACAGTGAAG;
PDK1:
forward:
5′-CATGTCACGCTGGGTAATGAGG,
reverse:
5′-CTCAACACGAGGTCTTGGTGCA-3′;
PGK1:
forward:
5′-CCGCTTTCATGTGGAGGAAGAAG-3′,
reverse:
5′-CTCTGTGAGCAGTGCCAAAAGC-3′;
VEGFA:
forward:
5′-TTGCCTTGCTGCTCTACCTCCA-3′,
reverse:
5′-GATGGCAGTAGCTGCGCTGATA-3′.

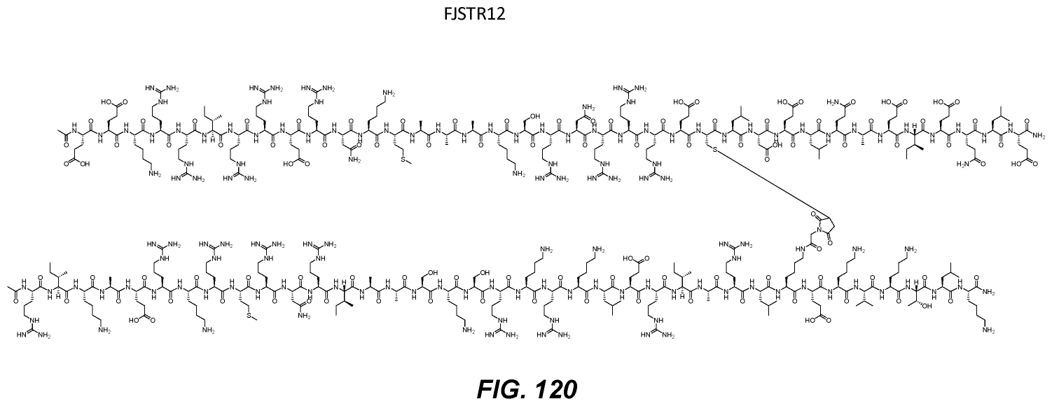

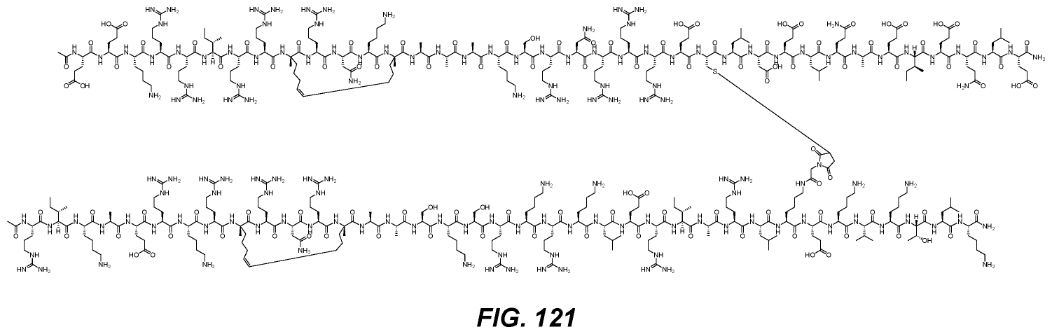

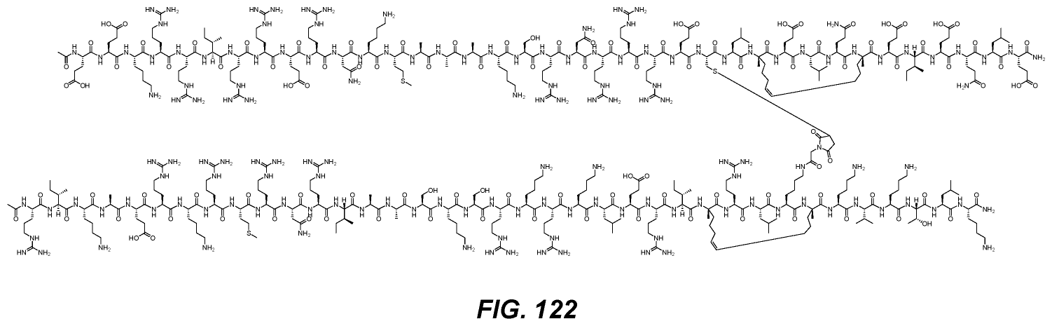

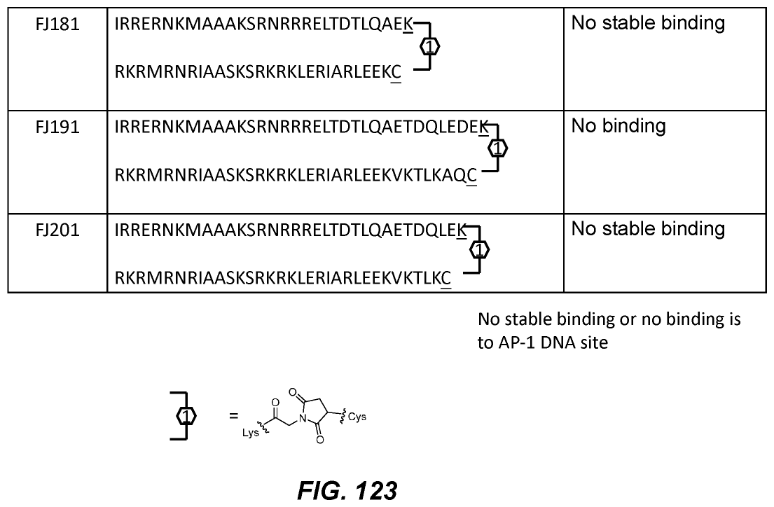

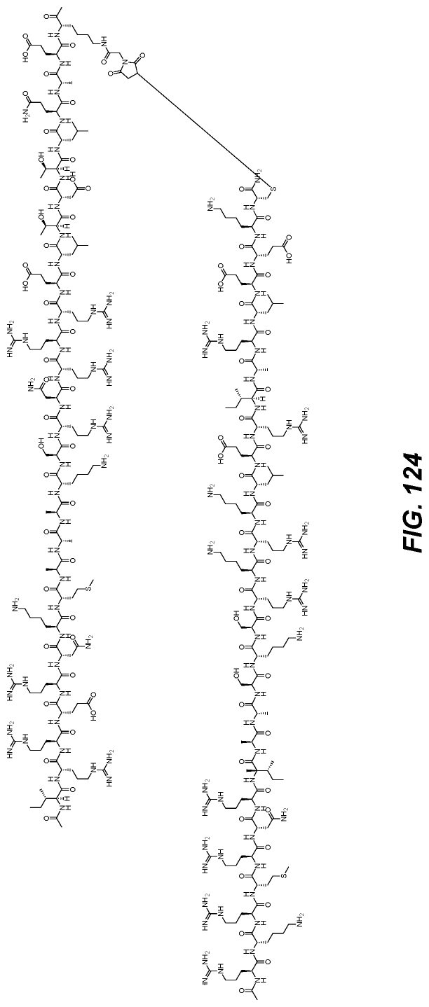

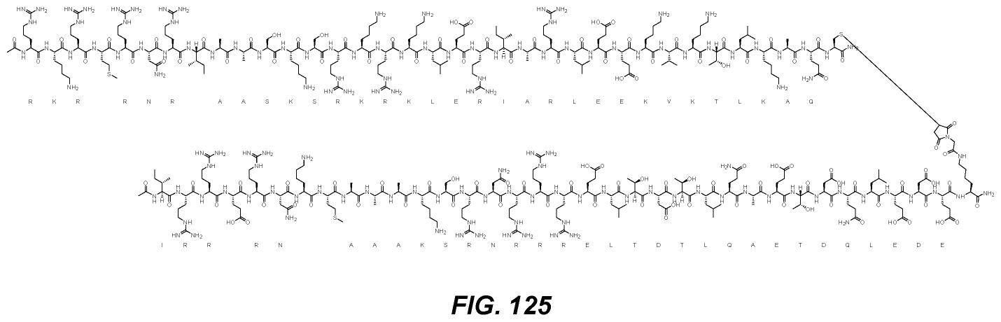

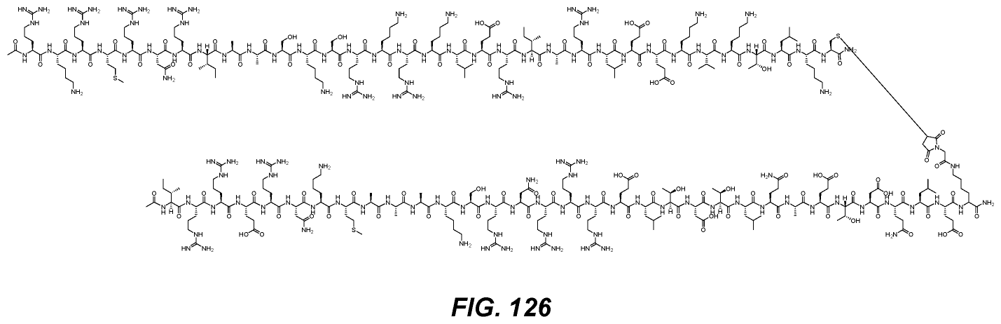

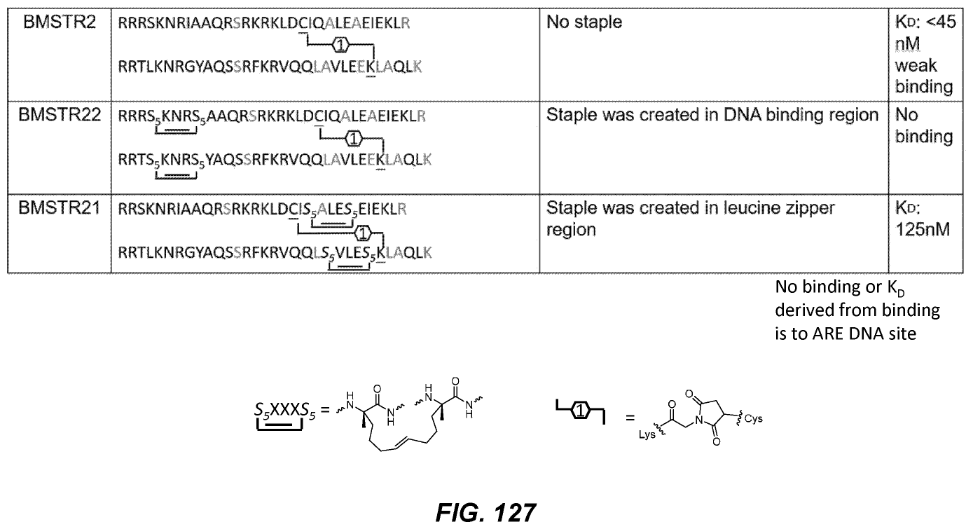

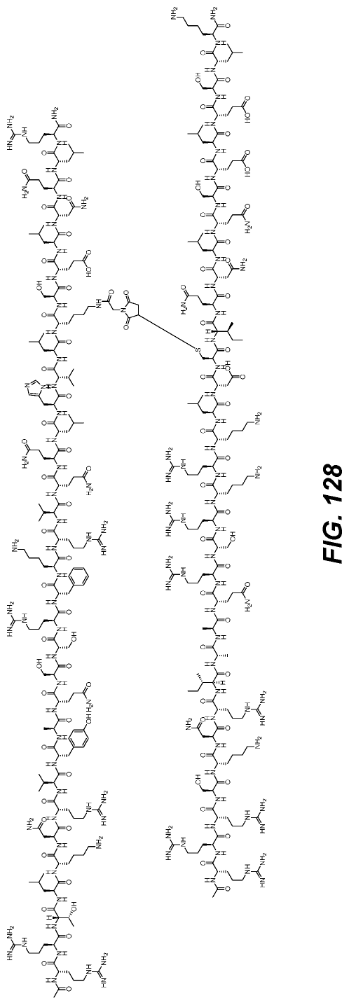

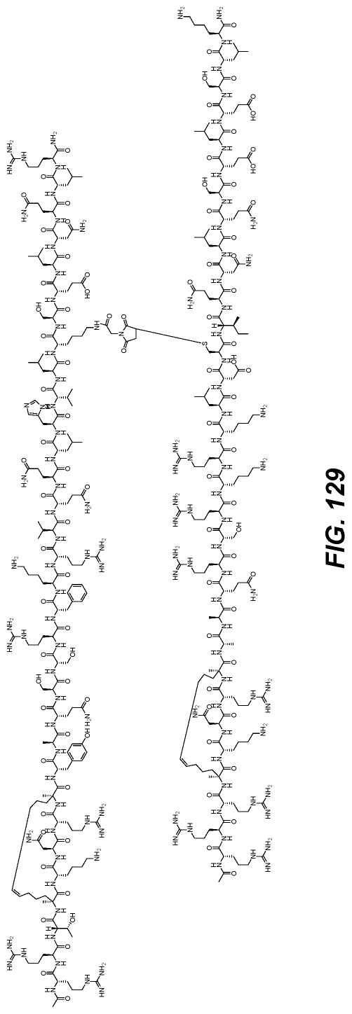

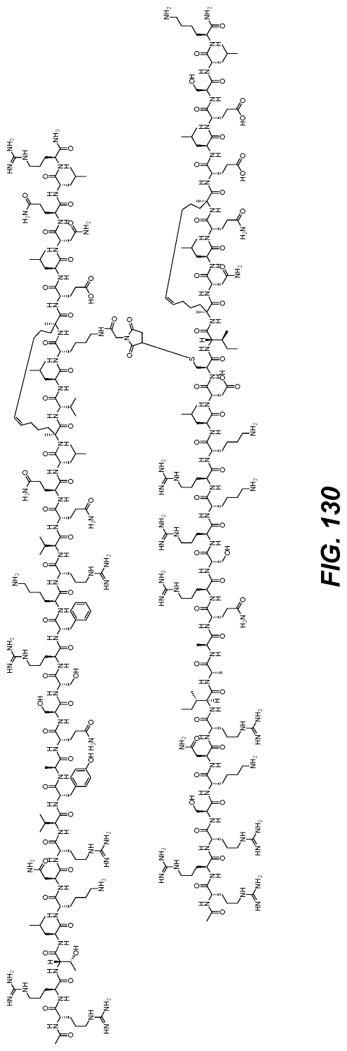

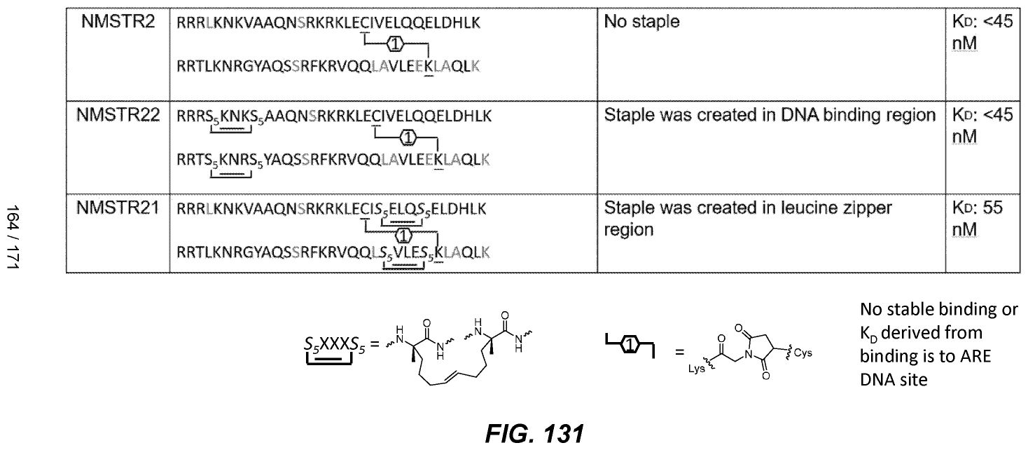

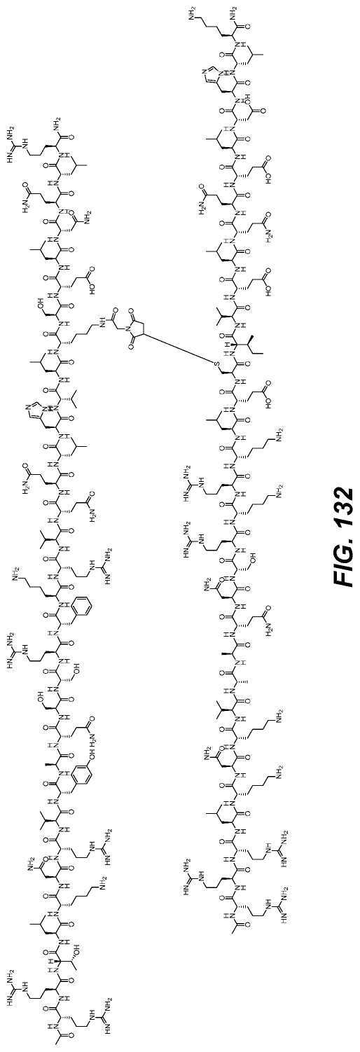

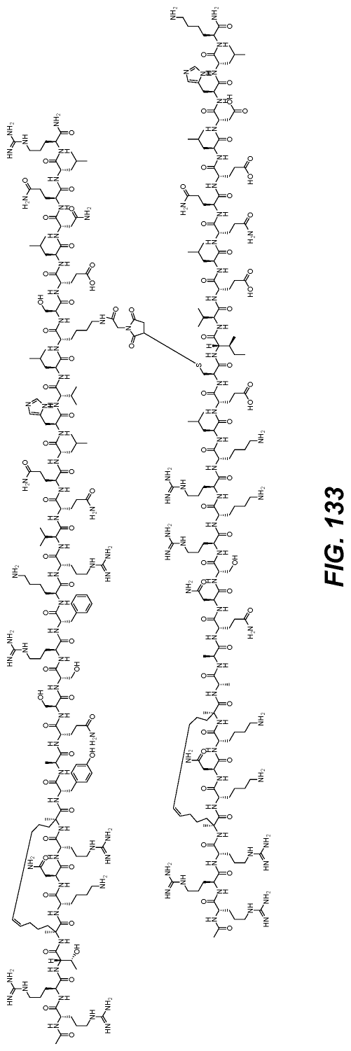

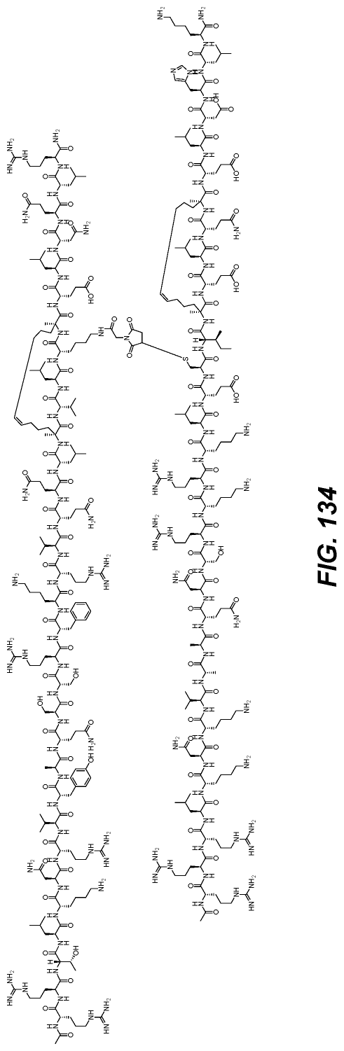

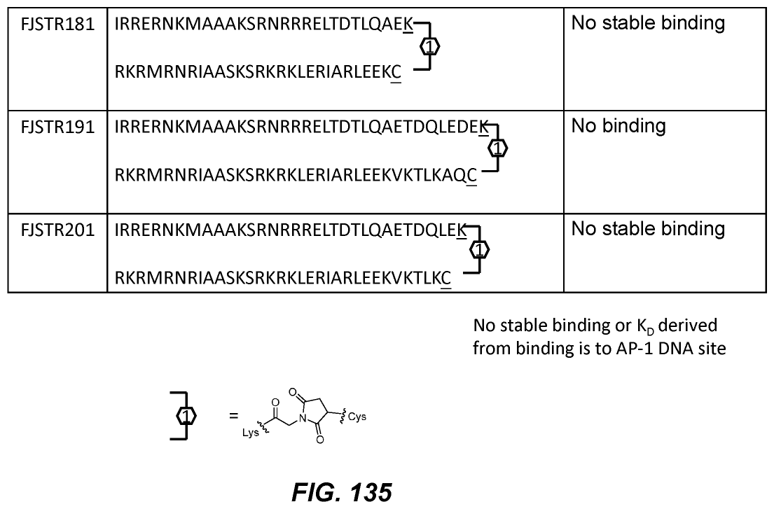

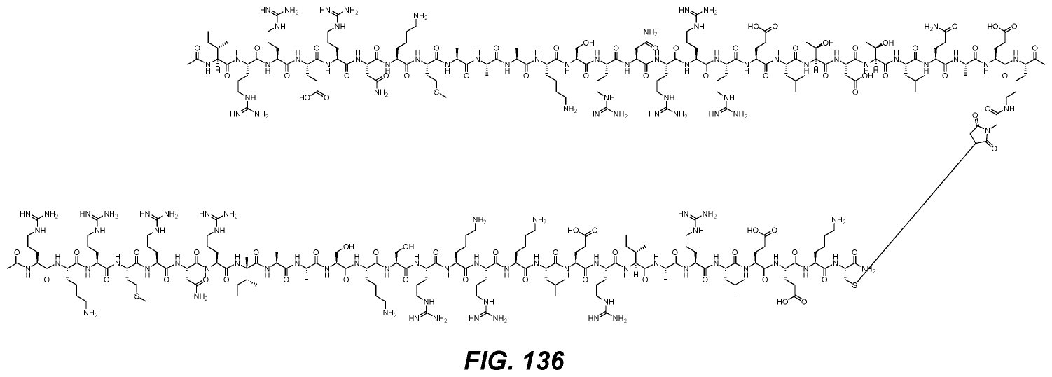

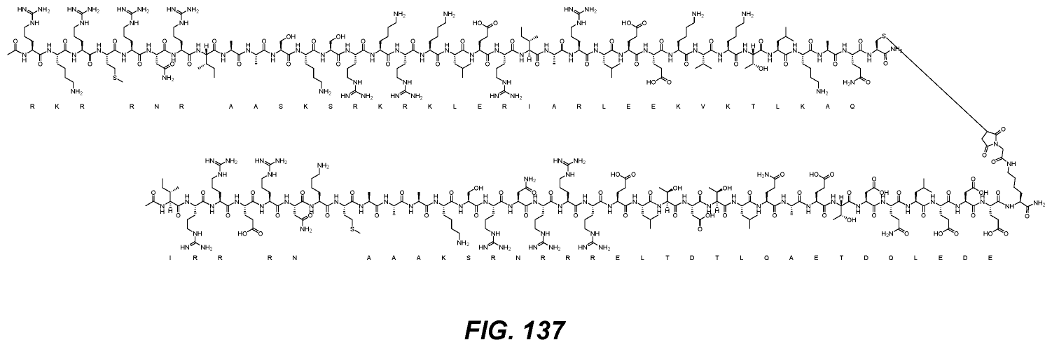

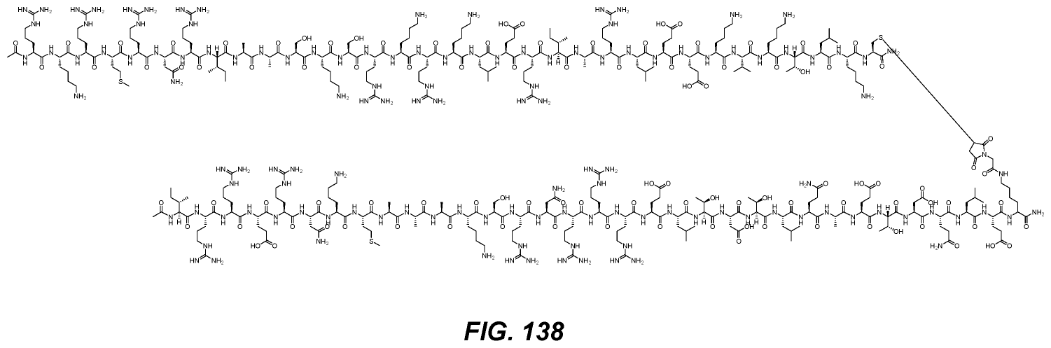

Trypan Blue Cell Viability Assay. MDA-MB-231 cells were treated with DMSO or 20 mM STR22 every 24 hours for 24 hours (FIG. 104A) or 120 hours (FIG. 104B) under 1% 02 and then trypsinized for 5 minutes at 37° C., 5% CO₂, 1% 02 and then quenched with a double volume of DMEM media. The cell suspension was then collected and subjected for trypan blue viability reading using trypan blue cell viability analyzer (Beckman Coulter Vi-CELL XR).

Boyden Chamber Invasion Assay. Each Boyden chamber membrane (Fisher Scientific, 353097) was coated with a thin layer of Basement Membrane Extract (BME, 200 μl of 0.25 mg/ml stock; 50 μg total BME per membrane) and incubated at 37° C. for 1 hr. Cells were trypsinized, neutralized in 10% FBS DMEM media, and centrifuged at 500×g for 5 min followed by two rounds of PBS washes to remove remaining serum-containing media. Cells were then resuspended in serum-free media and diluted to the desired concentration for plating onto the Boyden chamber. 1×10⁶ MDA-MB-231 cells in 300 μL serum-free media were plated on each Boyden chamber. 500 μL of 10% FBS DMEM media was placed in the lower well, acting as the chemoattractant. Serum-free media placed in the lower wells served as negative controls. Treated cells were plated in 20 μM of STR22. After a 24 hr incubation, the membranes were stained with Calcein AM (Fisher Scientific, 354217) for 1 hr at 37° C. to stain for live cells. The tops of the chambers were swabbed to remove remaining cells, and cells on the bottom of the chamber were dissociated from the membrane by incubating in cell dissociated buffer (R&D Systems, 3455-05-03) in a shaker at 37° C. for 1 hr. Calcein AM signal was measured in Perkin Elmer Victor X3 plate reader as a read-out of invaded cells.

Immunoblotting. Following indicated treatments, cells were first washed with ice-cold PBS. Whole-cell extracts were prepared by directly lysing cells with Laemmli sample buffer (Bio-rad, 1610747) supplemented with 2-Mercaptoethanol (Gibco, 21985023), protease inhibitor (Roche, 4693159001), PMSF (Roche, 10837091001) and phosphatase inhibitor (GB-450) at 4° C. Finally, protein samples were boiled at 95° C. for 5 mins. Western blotting was performed using antibodies for XBP1 (Biolegend, 9D11A43), HIF1α (Novus Biologicals, NB100-479), GAPDH (Santa Cruz Biotech, sc-32233) and α-tubulin (Invitrogen, MA1-19401) were used. Blots were imaged using Li—COR Odyssey Fc.

RNA Sequencing and Analysis. Total RNA was extracted from cell culture samples treated as described in the main text using the RNeasy Plus Mini Kit (Qiagen). Three independent biological replicates were performed per experimental condition for a total of 12 RNA samples. RNA sample quality check, library construction, and sequencing were performed by the University of Chicago Genomics Facility following standard protocols. The average RNA Integrity Score was 9.9. All 12 samples were sequenced in two runs on a NovaSeq 6000 sequencer to generate paired-end 100 bp reads. For each sample, raw FASTQ files from two flow cells were combined before downstream processing. RNA-seq data were analyzed as previously reported and briefly described below⁴⁷. A local Galaxy 20.05 instance was used for the following steps. Quality and adapter trimming were performed on the raw sequencing reads using Trim Galore! 0.6.3. The reads were mapped to the human genome (UCSC hg19 with GENCODE annotation) using RNA STAR 2.7.5b. The resulting mapped reads from each sample were counted by featureCounts 1.6.4 for per-gene read counts.

The raw counts were analyzed for differential expression between experimental conditions using DESeq2 1.22.1, which also generated a normalized gene expression matrix. Morpheus software (https://software.broadinstitute.org/morpheus) was used to draw gene expression heatmaps using the DESeq2-normalized gene expression data. For each gene, the normalized expression values of all samples were transformed by subtracting the mean and dividing by the standard deviation. The transformed gene expression values were used to generate heatmaps.

Gene Set Enrichment Analysis. Gene expression data normalized by DESeq2 from above were used for gene set enrichment analyses by GSEA v4.1.0^{48,49}. Specifically, M5891 HALLMARK_HYPOXIA, M13324 BIOCARTA_HIF_PATHWAY, and M4653

RESPONSE_TO_HYPOXIA gene sets were used to compare differences in hypoxic response between normoxia, hypoxia, and hypoxia+STR22 experimental conditions (FIG. 103D; FIGS. 109E-109F). A p-value and a normalized enrichment score was provided by GSEA for each comparison.

Clustering of Variable Genes. The top 5,000 most variable genes were selected, and the normalized gene expression data were analyzed by the Morpheus software. K-means clustering with 4 clusters was applied to the gene expression data of the RNA-seq experiment.

Chromatin Immunoprecipitation (ChIP) Sequencing and Analysis. DNA sample quality check, library construction, and sequencing were performed by the University of Chicago Genomics Facility following standard protocols. Samples were sequenced on a NovaSeq 6000 sequencer to generate paired-end 100 bp reads. RNA-seq data were analyzed using a local Galaxy

20.05 instance using the following steps: quality and adapter trimming were performed on the raw sequencing reads using Trim Galore! 0.6.3. IP and input reads for each sample were mapped to the human genome (UCSC hg19 with GENCODE annotation) using BWA-MEM 0.7.17.1⁵⁰. To visualize ChIP-seq results, the mapped reads files were counted and the resulting TDF files graphed using Integrative Genomics Viewer 2.9.4⁵¹. The mapped reads were converted to a SAM file format using samtools 1.2⁵².

HIF-1α Transcription Factor Binding Analysis. Peak calling, motif analysis, and annotations were performed by Homer 4.11.1 using the IP and input SAM files for each sample⁵³. Unique and overlapping peaks between the hypoxia and hypoxia+STR22 samples were determined based on whether peak centers were within 100 bp distance. Homer was also used to detect the presence of the HRE motif CACGT within the hypoxia sample peaks. DeepTools 3.3.2 was used to compare differences in HIF-1 binding with or without STR treatment in hypoxia⁵⁴. Specifically, each sample's IP reads were compared to its input reads and then normalized to total read count. Signals at each binding peak were calculated and then plotted as a heatmap for each sample. Average HIF-1 signal for the normoxia, hypoxia, and hypoxia+STR22 samples were determined by calculating the average across all peaks for each sample using the peak coordinates of the hypoxia sample. The signal of the normoxia sample was deducted as background before comparing the hypoxia and hypoxia+STR22 samples for differential binding using Graphpad Prism 9.3.1 (GraphPad Software) to perform paired t-test and area under the curve analyses. To compare ChIP-seq results from two independent biological replicates, DeepTools was also used to generate read coverage tables from sequencing data of two independent biological replicates.

Mouse Xenograft Studies. All animal protocols related to mouse experiments were approved by the University of Chicago Institutional Animal Care and Use Committee (IACUC #72439). Approximately 1×10⁶ human triple-negative breast cancer cells (MDA-MB-231) or 2×10⁶ human triple-negative breast cancer cells (fate mapping MDA-MB-231) in 100 μL PBS were injected into the fourth mammary fat pad of 8-10 week old female athymic nude mice (Charles River). When tumors reached approximately 100 mm3 in volume, mice were randomized into groups for twice-weekly intratumoral injections of STR22 (15 μg in 20 μL PBS; n=5 for MDA-MB-231 or 30 μg in 20 μL PBS; n=10) or Vehicle (20 μL PBS; n=5 for MDA-MB-231 or n=10 for fate mapping MDA-MB-231). Tumor growth was monitored twice a week using digital caliper measurements in two dimensions (A, B) to estimate volume. Tumor volume was calculated as: (A*B²)/2, where B is the largest diameter and A is the diameter perpendicular to B. Tumor growth (V/V₀) or final volume/mass was shown as mean+/−s.e.m. with P values determined by multiple unpaired t-tests. Statistical outliers (defined as greater than 3 deviations from the mean) were identified and excluded. For xenograft tumor gene expression analysis, mice were sacrificed 24 hours after the final treatment and tumors were dissected and homogenized in Trizol using gentleMACS™ M tubes (Miltenyi Biotech). Total RNA was isolated using the Direct-Zol™ RNA Miniprep Plus kit (Zymo Research). Real-time PCR was carried out as described above.

### Example 14—Pharmacokinetics of STR22

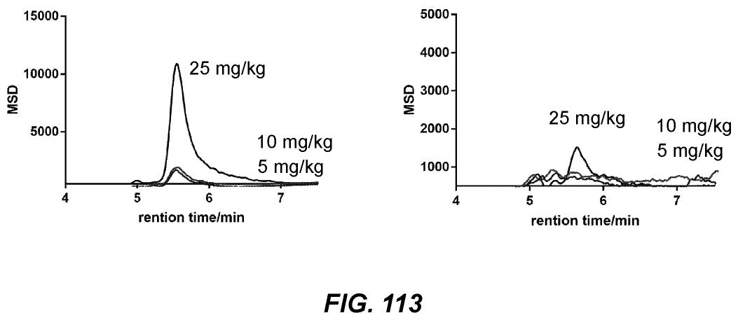

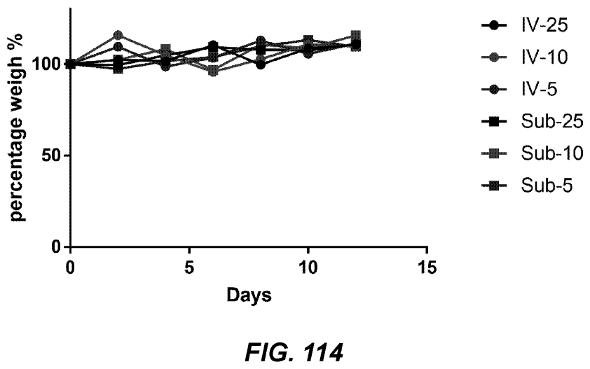

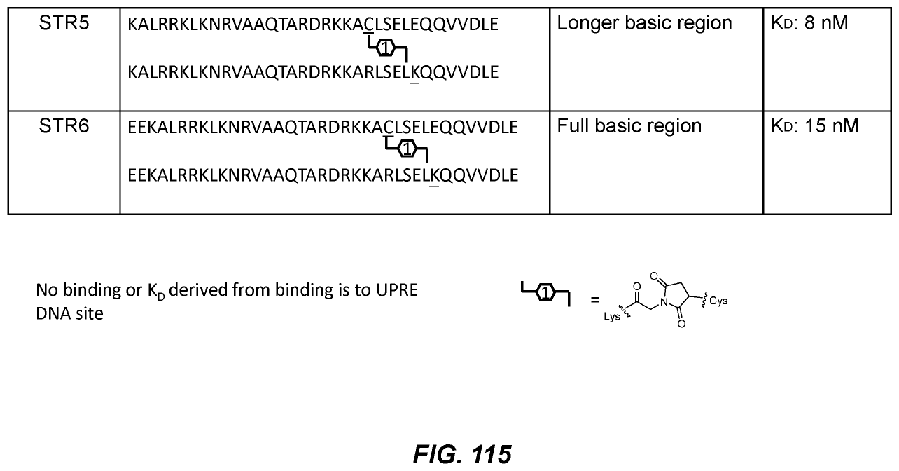

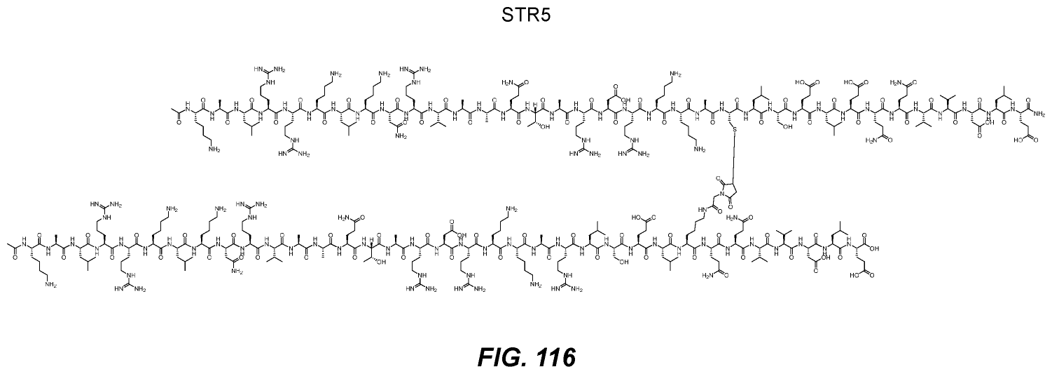

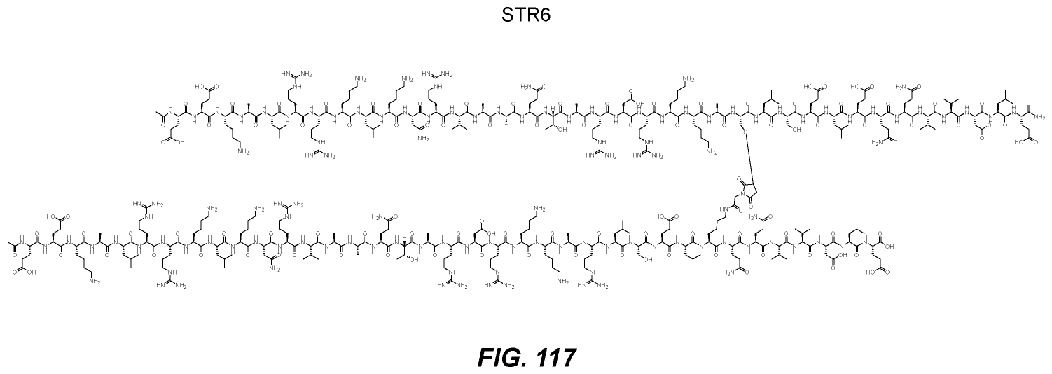

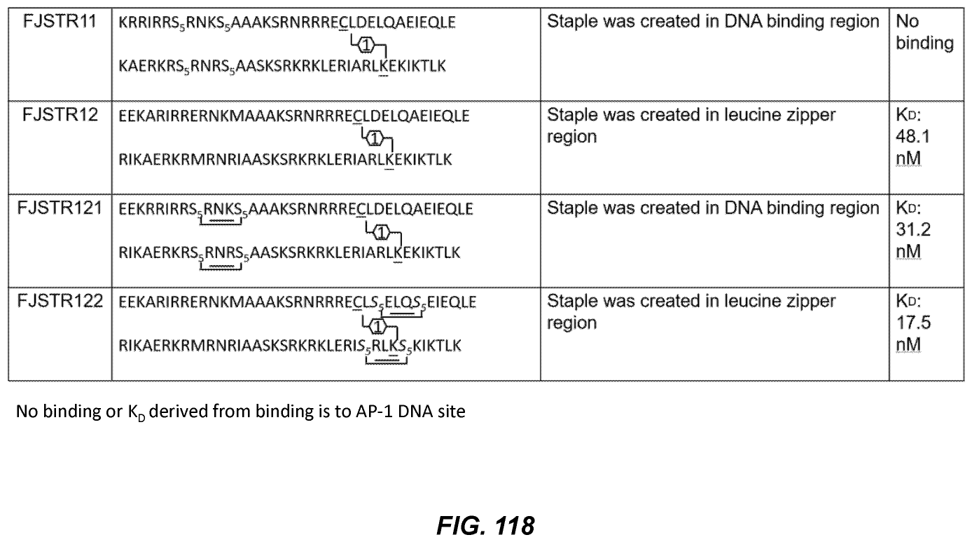

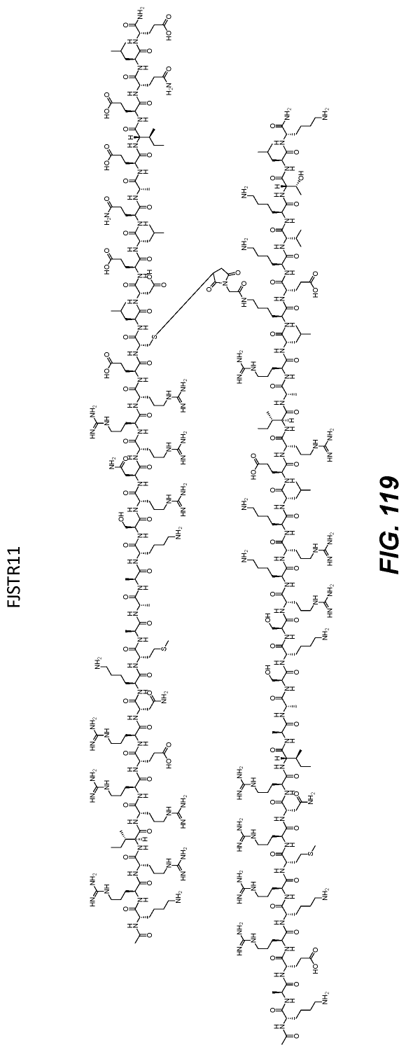

STR22 at various provided concentrations were added to 10× diluted plasma then added to 4× volume of methanol, centrifuged and used for LCMS detection (FIG. 112). Injection of different concentrations of STR22 was performed through either intravenous or subcutaneous. Blood was collected through submandibular bleed at different time points and analyze via established extraction and LCMS methods. FIG. 113 demonstrates STR22 can be detected in blood following IP and IV injections. Body weight change after a single injection of STR22 through intravenous (IV) or subcutaneous (Sub) was also measured. No changes in activity or body weight observed (FIG. 114).

All of the methods disclosed and claimed herein can be made and executed without undue experimentation in light of the present disclosure. While the compositions and methods of this invention have been described in terms of certain aspects, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The references cited herein, to the extent that they provide exemplary procedural or other details supplementary to those set forth herein, are specifically incorporated herein by reference.
1 Lambert, S. A. et al. The Human Transcription Factors. Cell 175, 598-599, doi:10.1016/j.cell.2018.09.045 (2018).
2 Lee, T. I. & Young, R. A. Transcriptional regulation and its misregulation in disease. Cell 152, 1237-1251, doi:10.1016/j.cell.2013.02.014 (2013).
3 Darnell, J. E., Jr. Transcription factors as targets for cancer therapy. Nat Rev Cancer 2, 740-749, doi:10.1038/nrc906 (2002).
4 Lee, A. H., Iwakoshi, N. N. & Glimcher, L. H. XBP-1 regulates a subset of endoplasmic reticulum resident chaperone genes in the unfolded protein response. Mol Cell Biol 23, 7448-7459 (2003).
5 Kim, J. W., Gao, P. & Dang, C. V. Effects of hypoxia on tumor metabolism. Cancer Metastasis Rev 26, 291-298, doi:10.1007/s10555-007-9060-4 (2007).
6 Liao, D. & Johnson, R. S. Hypoxia: a key regulator of angiogenesis in cancer. Cancer Metastasis Rev 26, 281-290, doi:10.1007/s10555-007-9066-y (2007).
7 Semenza, G. L. Targeting HIF-1 for cancer therapy. Nat Rev Cancer 3, 721-732, doi:10.1038/nrc1187 (2003).
8 Gilkes, D. M., Semenza, G. L. & Wirtz, D. Hypoxia and the extracellular matrix: drivers of tumour metastasis. Nat Rev Cancer 14, 430-439, doi:10.1038/nrc3726 (2014).
9 Rapisarda, A. & Melillo, G. Overcoming disappointing results with antiangiogenic therapy by targeting hypoxia. Nat Rev Clin Oncol 9, 378-390, doi:10.1038/nrclinonc.2012.64 (2012).
10 Chen, X. & Cubillos-Ruiz, J. R. Endoplasmic reticulum stress signals in the tumour and its microenvironment. Nat Rev Cancer 21, 71-88, doi:10.1038/s41568-020-00312-2 (2021).
11 Avril, T., Vauleon, E. & Chevet, E. Endoplasmic reticulum stress signaling and chemotherapy resistance in solid cancers. Oncogenesis 6, e373, doi:10.1038/oncsis.2017.72 (2017).
12 Chen, S. et al. The emerging role of XBP1 in cancer. Biomed Pharmacother 127, 110069, doi:10.1016/j.biopha.2020.110069 (2020).
13 Reinke, A. W., Baek, J., Ashenberg, O. & Keating, A. E. Networks of bZIP protein-protein interactions diversified over a billion years of evolution. Science 340, 730-734, doi:10.1126/science.1233465 (2013).
14 Bersten, D. C., Sullivan, A. E., Peet, D. J. & Whitelaw, M. L. bHLH-PAS proteins in cancer. Nat Rev Cancer 13, 827-841, doi:10.1038/nrc3621 (2013).
15 Chen, X. et al. XBP1 promotes triple-negative breast cancer by controlling the HIF1α lpha pathway. Nature 508, 103-107, doi:10.1038/nature13119 (2014).
16 Lin, A. et al. The LINK-A lncRNA activates normoxic HIF1α lpha signalling in triple-negative breast cancer. Nat Cell Biol 18, 213-224, doi:10.1038/ncb3295 (2016).
17 Briggs, K. J. et al. Paracrine Induction of HIF by Glutamate in Breast Cancer: EglN1 Senses Cysteine. Cell 166, 126-139, doi:10.1016/j.cell.2016.05.042 (2016).
18 Talanian, R. V., McKnight, C. J. & Kim, P. S. Sequence-specific DNA binding by a short peptide dimer. Science 249, 769-771, doi:10.1126/science.2389142 (1990).
19 Kouzarides, T. & Ziff, E. Leucine zippers of fos, jun and GCN4 dictate dimerization specificity and thereby control DNA binding. Nature 340, 568-571, doi:10.1038/340568a0 (1989).
20 Lynne E. Canne, Adrian R. Ferre-D'Amare, Stephen K. Burley & Kent, S. B. H. Total Chemical Synthesis of a Unique Transcription Factor-Related Protein: cMyc-Max. Journal of the American Chemical Society 117, 2998-3007 (1995).
21 Metallo, S. J. & Schepartz, A. Certain bZIP peptides bind DNA sequentially as monomers and dimerize on the DNA. Nat Struct Biol 4, 115-117, doi:10.1038/nsb0297-115 (1997).
22 Blancafort, P., Segal, D. J. & Barbas, C. F., 3rd. Designing transcription factor architectures for drug discovery. Mol Pharmacol 66, 1361-1371, doi:10.1124/mol.104.002758 (2004).
23 Sonia Boga, David Bouzada, Diego Garcia Pena, Miguel Vázquez López & Vázquez, M. E. Sequence-Specific DNA Recognition with Designed Peptides. Eur. J. Org. Chem., 249-261 (2018).
24 Madden, S. K., de Araujo, A. D., Gerhardt, M., Fairlie, D. P. & Mason, J. M. Taking the Myc out of cancer: toward therapeutic strategies to directly inhibit c-Myc. Mol Cancer 20, 3, doi:10.1186/s12943-020-01291-6 (2021).
25 Speltz, T. E. et al. Targeting MYC with modular synthetic transcriptional repressors derived from bHLH DNA-binding domains. Nature biotechnology, doi:10.1038/s41587-022-01504-x (2022).
26 Schafmeister, C. E., Po, J. & Verdine, G. L. An All-Hydrocarbon Cross-Linking System for Enchancing the Helicity and Metabolic Stability of Peptides. Journal of the American Chemical Society 122, 5891-5892 (2000).
27 Mason, J. M., Schmitz, M. A., Muller, K. M. & Arndt, K. M. Semirational design of Jun-Fos coiled coils with increased affinity: Universal implications for leucine zipper prediction and design. Proc Natl Acad Sci USA 103, 8989-8994, doi:10.1073/pnas.0509880103 (2006).
28 Chu, Q. et al. Towards understanding cell penetration by stapled peptides. MedChemComm 6, 111-119 (2015).
29 Bird, G. H. et al. Biophysical determinants for cellular uptake of hydrocarbon-stapled peptide helices. Nature chemical biology 12, 845-852, doi:10.1038/nchembio.2153 (2016).
30 LaRochelle, J. R., Cobb, G. B., Steinauer, A., Rhoades, E. & Schepartz, A. Fluorescence correlation spectroscopy reveals highly efficient cytosolic delivery of certain penta-arg proteins and stapled peptides. Journal of the American Chemical Society 137, 2536-2541, doi:10.1021/ja510391n (2015).
31 Walensky, L. D. et al. Activation of apoptosis in vivo by a hydrocarbon-stapled BH3 helix. Science 305, 1466-1470, doi:10.1126/science.1099191 (2004).
32 Bernal, F., Tyler, A. F., Korsmeyer, S. J., Walensky, L. D. & Verdine, G. L. Reactivation of the p53 tumor suppressor pathway by a stapled p53 peptide. Journal of the American Chemical Society 129, 2456-2457, doi:10.1021/ja0693587 (2007).
33 Moellering, R. E. et al. Direct inhibition of the NOTCH transcription factor complex. Nature 462, 182-188, doi:10.1038/nature08543 (2009).
34 Kong, X. D. et al. De novo development of proteolytically resistant therapeutic peptides for oral administration. Nat Biomed Eng 4, 560-571, doi:10.1038/s41551-020-0556-3 (2020).
35 Bird, G. H. et al. Hydrocarbon double-stapling remedies the proteolytic instability of a lengthy peptide therapeutic. Proceedings of the National Academy of Sciences of the United States of America 107, 14093-14098, doi:10.1073/pnas.1002713107 (2010).
36 Yang, P. Y. et al. Stapled, Long-Acting Glucagon-like Peptide 2 Analog with Efficacy in Dextran Sodium Sulfate Induced Mouse Colitis Models. Journal of medicinal chemistry 61, 3218-3223, doi:10.1021/acs.jmedchem.7b00768 (2018).
37 Chang, Y. S. et al. Stapled alpha-helical peptide drug development: A potent dual inhibitor of MDM2 and MDMX for p53-dependent cancer therapy. Proceedings of the National Academy of Sciences of the United States of America 110, E3445-3454, doi:10.1073/pnas.1303002110 (2013).
38 Wang, G. L. & Semenza, G. L. Purification and characterization of hypoxia-inducible factor 1. J Biol Chem 270, 1230-1237, doi:10.1074/jbc.270.3.1230 (1995).
39 Ye, I. C. et al. Molecular Portrait of Hypoxia in Breast Cancer: A Prognostic Signature and Novel HIF-Regulated Genes. Mol Cancer Res 16, 1889-1901, doi:10.1158/1541-7786.MCR-18-0345 (2018).
40 Bharti, S. K. et al. Metabolic consequences of HIF silencing in a triple negative human breast cancer xenograft. Oncotarget 9, 15326-15339, doi:10.18632/oncotarget.24569 (2018).
41 Vaupel, P., Mayer, A. & Hockel, M. Tumor hypoxia and malignant progression. Methods Enzymol 381, 335-354, doi:10.1016/S0076-6879(04)81023-1 (2004).
42 Cosse, J. P. & Michiels, C. Tumour hypoxia affects the responsiveness of cancer cells to chemotherapy and promotes cancer progression. Anticancer Agents Med Chem 8, 790-797, doi:10.2174/187152008785914798 (2008).
43 Godet, I. et al. Fate-mapping post-hypoxic tumor cells reveals a ROS-resistant phenotype that promotes metastasis. Nat Commun 10, 4862, doi:10.1038/s41467-019-12412-1 (2019).
44 Fallah, J. et al. FDA Approval Summary: Belzutifan for von Hippel-Lindau disease associated tumors. Clinical cancer research: an official journal of the American Association for Cancer Research, doi:10.1158/1078-0432.CCR-22-1054 (2022).
45 Kim, Y. W., Grossmann, T. N. & Verdine, G. L. Synthesis of all-hydrocarbon stapled alpha-helical peptides by ring-closing olefin metathesis. Nature protocols 6, 761-771, doi:10.1038/nprot.2011.324 (2011).
46 Schneider, C. A., Rasband, W. S. & Eliceiri, K. W. NIH Image to ImageJ: 25 years of image analysis. Nat Methods 9, 671-675, doi:10.1038/nmeth.2089 (2012).
47 Nguyen, L. C. et al. Cannabidiol inhibits SARS-CoV-2 replication through induction of the host ER stress and innate immune responses. Sci Adv, eabi6110 (2022).
48 Subramanian, A. et al. Gene set enrichment analysis: a knowledge-based approach for interpreting genome-wide expression profiles. Proceedings of the National Academy of Sciences of the United States of America 102, 15545-15550, doi:10.1073/pnas.0506580102 (2005).
49 Liberzon, A. et al. The Molecular Signatures Database (MSigDB) hallmark gene set collection. Cell Syst 1, 417-425, doi:10.1016/j.cels.2015.12.004 (2015).
50 Li, H. Aligning sequence reads, clone sequences and assembly contigs with BWA-MEM. arXiv:1303.3997 (2013). <https://ui.adsabs.harvard.edu/abs/2013arXiv1303.3997L>.
51 Robinson, J. T. et al. Integrative genomics viewer. Nat Biotechnol 29, 24-26, doi:10.1038/nbt.1754 (2011).
52 Li, H. et al. The Sequence Alignment/Map format and SAMtools. Bioinformatics 25, 2078-2079, doi:10.1093/bioinformatics/btp352 (2009).
53 Heinz, S. et al. Simple combinations of lineage-determining transcription factors prime cis-regulatory elements required for macrophage and B cell identities. Mol Cell 38, 576-589, doi:10.1016/j.molcel.2010.05.004 (2010).
54 Ramirez, F., Dundar, F., Diehl, S., Gruning, B. A. & Manke, T. deepTools: a flexible platform for exploring deep-sequencing data. Nucleic Acids Res 42, W187-191, doi:10.1093/nar/gku365 (2014).

1. An engineered DNA-binding dimer comprising:
(a) a first engineered peptide comprising (i) a modified basic domain sequence of a first bZIP protein and (ii) a modified leucine zipper domain sequence of the first bZIP protein; and,
(b) a second engineered peptide linked to the first engineered peptide via a side-by-side interpeptide linkage, the second engineered peptide comprising (i) a modified basic domain sequence of a second bZIP protein and (ii) a modified leucine zipper domain sequence of the second bZIP protein,
wherein each of the first and second bZIP proteins, individually, have an isoleucine, leucine, or valine at position “a” of their respective leucine zipper domain sequences, or
wherein each of the first and second bZIP proteins, individually, have an isoleucine, leucine, or valine at position “d” of their respective leucine zipper domain sequences.

2. The engineered DNA-binding dimer of claim 1, wherein each of the first and second bZIP proteins, individually, have an isoleucine, leucine, or valine at position “a” of their respective leucine zipper domain sequences.

3. The engineered DNA-binding dimer of claim 2, wherein each of the first and second bZIP proteins, individually, have an isoleucine, leucine, or valine at position “d” of their respective leucine zipper domain sequences.

4. The engineered DNA-binding dimer of any one of claims 1 to 3, wherein each of the first and second bZIP proteins, individually, have a leucine at position “a” of their respective leucine zipper domain sequences.

5. The engineered DNA-binding dimer of any one of claims 1 to 3, wherein each of the first and second bZIP proteins, individually, have an isoleucine at position “a” of their respective leucine zipper domain sequences.

6. The engineered DNA-binding dimer of any one of claims 1 to 5, wherein each of the first and second bZIP proteins, individually, have a leucine at position “d” of their respective leucine zipper domain sequences.

7. The engineered DNA-binding dimer of any one of claims 1 to 5, wherein each of the first and second bZIP proteins, individually, have an isoleucine at position “d” of their respective leucine zipper domain sequences.

8. The engineered DNA-binding dimer of any one of claims 1 to 7, wherein:
a glutamine is present at position “e” of the leucine zipper domain sequence of the first bZIP protein and a glutamine is present at position “g” of the leucine zipper domain sequence of the second bZIP protein; or
an arginine is present at position “e” of the leucine zipper domain sequence of the first bZIP protein and a glutamic acid is present at position “g” of the leucine zipper domain sequence of the second bZIP protein.

9. The engineered DNA-binding dimer of any one of claims 1 to 7, wherein:
an arginine is present at position “e” of the leucine zipper domain sequence of the second bZIP protein and a glutamic acid is present at position “g” of the leucine zipper domain sequence of the first bZIP protein.

10. The engineered DNA-binding dimer of any one of claims 1 to 9, wherein at least one or both of the leucine zipper domain sequences of the first and/or second bZIP proteins have an alanine at least at one or more of positions “b”, “c”, or “f”.

11. The engineered DNA-binding dimer of any one of claims 1 to 10, wherein the modified basic domain sequence of the first bZIP protein is at most 25 residues in length.

12. The engineered DNA-binding dimer of claim 11, wherein the modified basic domain sequence of the first bZIP protein is 20 residues in length.

13. The engineered DNA-binding dimer of claim 1, wherein the modified leucine zipper domain sequence of the first bZIP protein is at most 15 residues in length.

14. The engineered DNA-binding dimer of claim 13, wherein the modified leucine zipper domain sequence of the first bZIP protein is 12 residues in length.

15. The engineered DNA-binding dimer of any of claims 1-14, wherein the first engineered peptide is at most 40 residues in length.

16. The engineered DNA-binding dimer of claim 15, wherein the first engineered peptide is 32 residues in length.

17. The engineered DNA-binding dimer of any of claims 1-16, wherein the modified basic domain sequence of the second bZIP protein is at most 25 residues in length.

18. The engineered DNA-binding dimer of claim 17, wherein the modified basic domain sequence of the second bZIP protein is 20 residues in length.

19. The engineered DNA-binding dimer of any of claims 1-18, wherein the modified leucine zipper domain sequence of the second bZIP protein is at most 15 residues in length.

20. The engineered DNA-binding dimer of claim 19, wherein the modified leucine zipper domain sequence of the second bZIP protein is 12 residues in length.

21. The engineered DNA-binding dimer of any of claims 1-20, wherein the second engineered peptide is at most 40 residues in length.

22. The engineered DNA-binding dimer of claim 21, wherein the second engineered peptide is 32 residues in length.

23. The engineered DNA-binding dimer of any of claims 1-22, wherein the modified basic domain sequence of the first bZIP protein comprises a serine substituted for any cysteine relative to a native basic domain sequence of the first bZIP protein.

24. The engineered DNA-binding dimer of any of claims 1-23, wherein the modified basic domain sequence of the second bZIP protein comprises a serine substituted for any cysteine relative to a native basic domain sequence of the second bZIP protein.

25. The engineered DNA-binding dimer of any of claims 1-24, wherein the modified leucine zipper domain sequence of the first bZIP protein comprises an alanine substituted for any cysteine at a “b”, “c”, or “f” position relative to a native leucine zipper domain sequence of the first bZIP protein.

26. The engineered DNA-binding dimer of any of claims 1-25, wherein the modified leucine zipper domain sequence of the first bZIP protein comprises a leucine substituted for any cysteine at an “a” or “d” position relative to a native leucine zipper domain sequence of the first bZIP protein.

27. The engineered DNA-binding dimer of any of claims 1-26, wherein the modified leucine zipper domain sequence of the second bZIP protein comprises an alanine substituted for any cysteine at a “b”, “c”, or “f” position relative to a native leucine zipper domain sequence of the second bZIP protein.

28. The engineered DNA-binding dimer of any of claims 1-27, wherein the modified leucine zipper domain sequence of the second bZIP protein comprises a leucine substituted for any cysteine at an “a” or “d” position relative to a native leucine zipper domain sequence of the second bZIP protein.

29. The engineered DNA-binding dimer of any of claims 1-28, wherein the modified basic domain sequence of the first bZIP protein comprises a cysteine at a position corresponding to the last position of a native basic domain sequence of the first bZIP protein.

30. The engineered DNA-binding dimer of claim 29, wherein the cysteine is a natural cysteine.

31. The engineered DNA-binding dimer of claim 29, wherein the cysteine is a modified cysteine.

32. The engineered DNA-binding dimer of any of claims 1-31, wherein the modified leucine zipper domain sequence of the second bZIP protein comprises a lysine at a position corresponding to a first “e” position of a native leucine zipper domain sequence of the second bZIP protein.

33. The engineered DNA-binding dimer of any of claims 29-32, wherein the side-by-side interpeptide linkage is between the cysteine and the lysine.

34. The engineered DNA-binding dimer of any of claims 1-33, wherein the modified leucine zipper domain sequence of the first bZIP protein comprises a leucine in place of any residue at an “a” or “d” position that is not a leucine or isoleucine relative to a native leucine zipper domain of the first bZIP protein.

35. The engineered DNA-binding dimer of any of claims 1-34, wherein the modified leucine zipper domain sequence of the second bZIP protein comprises a leucine in place of any residue at an “a” or “d” position that is not a leucine or isoleucine relative to a native leucine zipper domain of the second bZIP protein.

36. The engineered DNA-binding dimer of any of claims 1-35, wherein the first and second bZIP proteins, individually, comprise one or more intrapeptide stabilizing linkages, wherein the intrapeptide stabilizing linkage(s) crosslinks two amino acids in an amino acid pair.

37. The engineered DNA-binding dimer of claim 36, wherein each amino acid of the amino acid pair is within an alpha helical segment of the first bZIP protein.

38. The engineered DNA-binding dimer of claim 36 or 37, wherein each amino acid of the amino acid pair is within an alpha helical segment of the second bZIP protein.

39. The engineered DNA-binding dimer of claim 37 or 38, wherein the two amino acids in the amino acid pair are at position i and i+4.

40. The engineered DNA-binding dimer of any of claims 37-39, wherein the two amino acids in the amino acid pair are at position i and i+7.

41. The engineered DNA-binding dimer of any of claims 37-40, wherein the hydrocarbon intrapeptide stabilizing linkage comprises an optionally substituted alkyl chain or an optionally substituted alkenyl chain stapling a pair of amino acids on the first or second bZIP protein.

42. The engineered DNA-binding dimer of ay one of claims 37-40, wherein the amino acids in the amino acid pair comprise S-2-(4′-pentenyl) alanine and/or R-2-(7′-octenyl) alanine.

43. The engineered DNA-binding dimer of any of claims 1-41, wherein the first and second bZIP protein comrpise an interpeptide linkage.

44. The engineered DNA-binding dimer of claim 43, wherein the interpeptide linkage is between non-terminal amino acids of the first and second bZIP protein.

45. The engineered DNA-binding dimer of claim 43 or 44, wherein the interpeptide linkage is between amino acids in a basic region of the first and second bZIP proteins.

46. The engineered DNA-binding dimer of any of claims 43-45, wherein the interpeptide linkage comprises maleamide.

47. The engineered DNA-binding dimer of any of claims 1-46, wherein the first bZIP protein is c-Fos.

48. The engineered DNA-binding dimer of claim 47, wherein the modified DNA-binding domain sequence of c-Fos comprises:
(SEQ ID NO: 16)
IRRERNKMAAAKSRNRRREC;
(SEQ ID NO: 17)
IRR#RNK#AAAKSRNRRREC;
(SEQ ID NO: 18)
EEKRRIRRERNKMAAAKSRNRRREC;
or
(SEQ ID NO: 19)
EEKRRIRR#RNK#AAAKSRNRRREC;
wherein # are intrapeptide stabilizing linkage sites which together form the structure

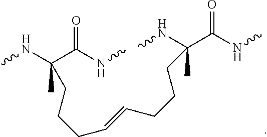

49. The engineered DNA-binding dimer of claim 47 or 48, wherein the modified leucine zipper domain sequence of c-Fos comprises:
(SEQ ID NO 20)
TDTLEDETDQLE;
(SEQ ID NO: 21)
LDELQAEIEQLE;
(SEQ ID NO: 22)
IDELQAEIEQLE;
(SEQ ID NO: 23)
IDEIQAEIEQIE;
(SEQ ID NO: 24)
L#ELQ#EIEQLE;
(SEQ ID NO: 25)
I#ELQ#EIEQLE;
or
(SEQ ID NO: 26)
I#EIQ#EIEQIE;
wherein # are intrapeptide stabilizing linkage sites which together form the structure

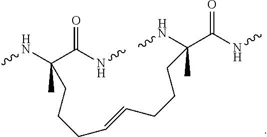

50. The engineered DNA-binding dimer of any of claims 1-49, wherein the second bZIP protein is c-Jun.

51. The engineered DNA-binding dimer of claim 50, wherein the modified DNA-binding domain sequence of c-Jun is:
(SEQ ID NO: 27)
RKRMRNRIAASKSRKRKLER;
(SEQ ID NO: 28)
RKR#RNR#AASKSRKRKLER;
(SEQ ID NO: 29)
RIKAERKRMRNRIAASKSRKRKLER;
or
(SEQ ID NO: 30)
RIKAERKR#RNR#AASKSRKRKLER;
wherein # are intrapeptide stabilizing linkage sites which together form the structure

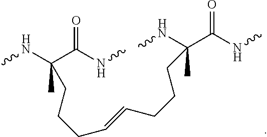

52. The engineered DNA-binding dimer of claim 50 or 51, wherein the modified leucine zipper domain sequence of c-Jun comprises:
(SEQ ID NO: 31)
IAKₘLEEKVKTLK;
(SEQ ID NO: 32)
IARLKₘEKVKTLK;
(SEQ ID NO: 33)
AAELKₘEKVATLK;
(SEQ ID NO: 34)
IARLKₘEKIKTLK;
(SEQ ID NO: 35)
IARIKₘEKIKTIK;
(SEQ ID NO: 36)
I#RLKₘ#KVKTLK;
or
(SEQ ID NO: 37)
I#RLKₘ#KIKTLK;
wherein
# are intrapeptide stabilizing linkage sites which together form the structure 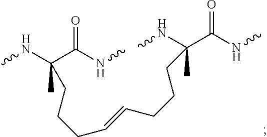
and
Kₘ is is a Lys residue attached to a maleimide-linker forming a portion of the structure:

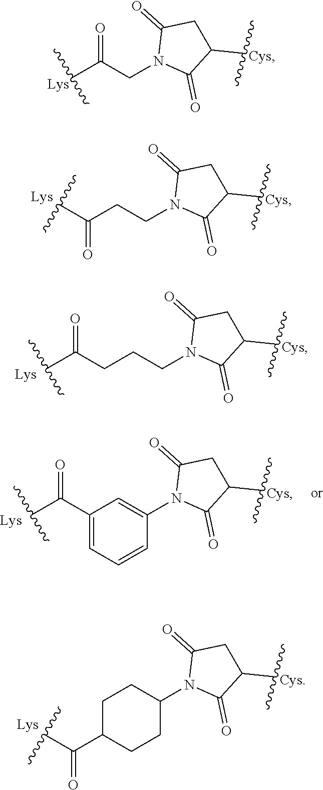 [SMILES: *C(=O)CN1C(=O)CC(*)C1=O ; *C(=O)CCN1C(=O)CC(*)C1=O ; *C(=O)CCCN1C(=O)CC(*)C1=O ; *C(=O)c1cccc(N2C(=O)CC(*)C2=O)c1 ; *C(=O)C1CCC(N2C(=O)CC(*)C2=O)CC1]

53. The engineered DNA-binding dimer of any of claims 1-35, wherein the first bZIP protein is XBP1.

54. The engineered DNA-binding dimer of claim 53, wherein the modified DNA-binding domain sequence of XBP1 comprises:
(SEQ ID NO: 38)
RRKLKNRVAAQTARDRKKAC;
or
(SEQ ID NO: 39)
RRK#KNR#AAQTARDRKKAC;
wherein # are intrapeptide stabilizing linkage sites which together form the structure

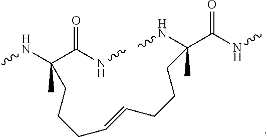

55. The engineered DNA-binding dimer of claim 53 or 54, wherein the modified leucine zipper domain sequence of XBP1 comprises:
(SEQ ID NO: 40)
MSELEQQVVDLE;
(SEQ ID NO: 41)
LSELEQQVVDLE;
(SEQ ID NO: 42)
L#ELE#QVVDLE;
wherein # are intrapeptide stabilizing linkage sites which together form the structure

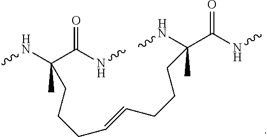

56. The engineered DNA-binding dimer of any of claims 53-55, wherein the second bZIP protein is XBP1.

57. The engineered DNA-binding dimer of any of claim 56, wherein the modified DNA-binding domain sequence of XBP1 comprises:
(SEQ ID NO: 43)
RRKLKNRVAAQTARDRKKAR;
or
(SEQ ID NO: 44)
RRK#KNR#AAQTARDRKKAR;
wherein # are intrapeptide stabilizing linkage sites which together form the structure

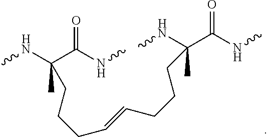

58. The engineered DNA-binding dimer of any of claim 56 or 57, wherein the modified leucine zipper domain sequence of XBP1 comprises:
(SEQ ID NO: 45)
MSELKₘQQVVDLE;
(SEQ ID NO: 46)
LSELKₘQQVVDLE;
or
(SEQ ID NO: 47)
L#ELKₘ#QVVDLE;
wherein
# are intrapeptide stabilizing linkage sites which together form the structure 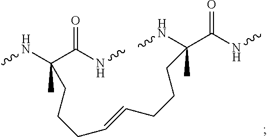
and
Kₘ is a Lys residue attached to a maleimide-linker forming a portion of the structure:

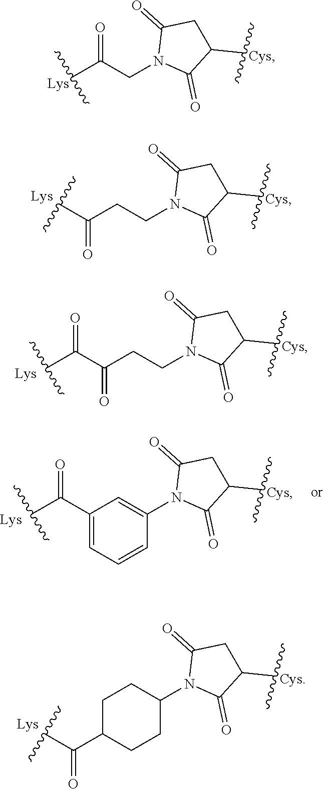 [SMILES: *C(=O)CN1C(=O)CC(*)C1=O ; *C(=O)CCN1C(=O)CC(*)C1=O ; *C(=O)C(=O)CCN1C(=O)CC(*)C1=O ; *C(=O)c1cccc(N2C(=O)CC(*)C2=O)c1 ; *C(=O)C1CCC(N2C(=O)CC(*)C2=O)CC1]

59. The engineered DNA-binding dimer of any of claims 1-35, wherein the first bZIP protein is ATF4.

60. The engineered DNA-binding dimer of claim 59, wherein the modified DNA-binding domain sequence of ATF4 comprises:
(SEQ ID NO: 48)
KKMEQNKTAATRYRQKKRAC;
wherein # are intrapeptide stabilizing linkage sites which together form the structure

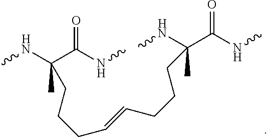

61. The engineered DNA-binding dimer of any claim 59 or 60, wherein the modified leucine zipper domain sequence of ATF4 comprises:
(SEQ ID NO: 49)
QEALTGELKELE;
(SEQ ID NO: 50)
LEALKAELKELR;
or
(SEQ ID NO: 51)
L#ALK#ELKELR.

62. The engineered DNA-binding dimer of any of claims 1-35, wherein the first bZIP protein is C/EBPβ.

63. The engineered DNA-binding dimer of claim 62, wherein the modified DNA-binding domain sequence of C/EBPβ comprises:
(SEQ ID NO: 52)
IRRERNNIAVRKSRDKAKMC;
wherein # are intrapeptide stabilizing linkage sites which together form the structure

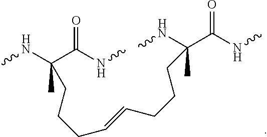

64. The engineered DNA-binding dimer of claim 62 or 63, wherein the modified leucine zipper domain sequence of C/EBPβ comprises:
(SEQ ID NO: 53)
LLELQHKVLELR;
or
(SEQ ID NO: 54)
L#ELQ#KVLELR;
wherein # are intrapeptide stabilizing linkage sites which together form the structure

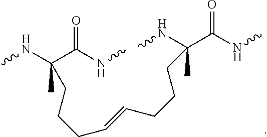

65. The engineered DNA-binding dimer of any of claims 59-64, wherein the second bZIP protein is ATF4.

66. The engineered DNA-binding dimer of claim 65, wherein the modified DNA-binding domain sequence of ATF4 comprises:
(SEQ ID NO: 55)
KKMEQNKTAATRYRQKKRAE;
wherein # are intrapeptide stabilizing linkage sites which together form the structure

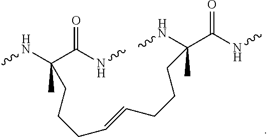

67. The engineered DNA-binding dimer of claim 65 or 66, wherein the modified leucine zipper domain sequence of ATF4 comprises:
(SEQ ID NO: 56)
QEALKₘGELKELE;
(SEQ ID NO: 57)
LEALKₘAELKELR;
or
(SEQ ID NO: 58)
L#ALKₘ#ELKELR;
wherein
# are intrapeptide stabilizing linkage sites which together form the structure 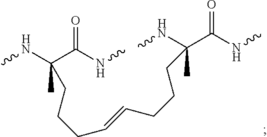
and
Kₘ is a Lys residue attached to a maleimide-linker forming a portion of the structure:

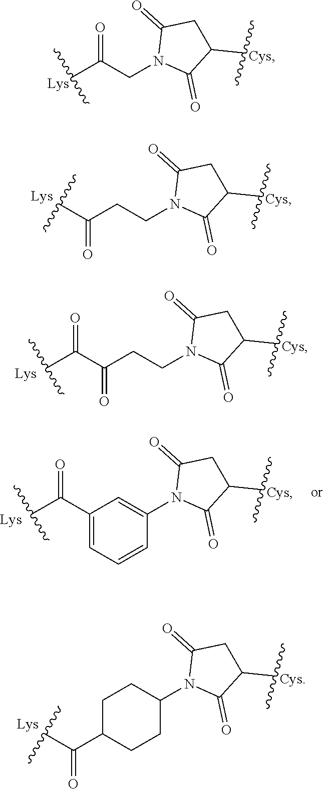 [SMILES: *C(=O)CN1C(=O)CC(*)C1=O ; *C(=O)CCN1C(=O)CC(*)C1=O ; *C(=O)C(=O)CCN1C(=O)CC(*)C1=O ; *C(=O)c1cccc(N2C(=O)CC(*)C2=O)c1 ; *C(=O)C1CCC(N2C(=O)CC(*)C2=O)CC1]

68. The engineered DNA-binding dimer of any of claims 1-67, wherein the first engineered peptide comprises a intrapeptide stabilizing linkage.

69. The engineered DNA-binding dimer of claim 68, wherein the intrapeptide stabilizing linkage is between the fourth position and the eighth position of the first engineered peptide.

70. The engineered DNA-binding dimer of claim 68, wherein the intrapeptide stabilizing linkage is between the twenty-second position and the twenty-sixth position of the first engineered peptide.

71. The engineered DNA-binding dimer of any of claims 1-68, wherein the second engineered peptide comprises a intrapeptide stabilizing linkage.

72. The engineered DNA-binding dimer of claim 71, wherein the intrapeptide stabilizing linkage is between the fourth position and the eighth position of the second engineered peptide.

73. The engineered DNA-binding dimer of claim 71, wherein the intrapeptide stabilizing linkage is between the twenty-second position and the twenty-sixth position of the second engineered peptide.

74. The engineered DNA-binding dimer of any of claims 1-73, wherein the side-by-side interpeptide linkage comprises a maleimide-thiol adduct.

75. The engineered DNA-binding dimer of claim 74, wherein the side-by-side interpeptide linkage is

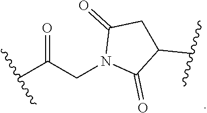 [SMILES: *C(=O)CN1C(=O)CC(*)C1=O]

76. An engineered DNA-binding dimer having formula:

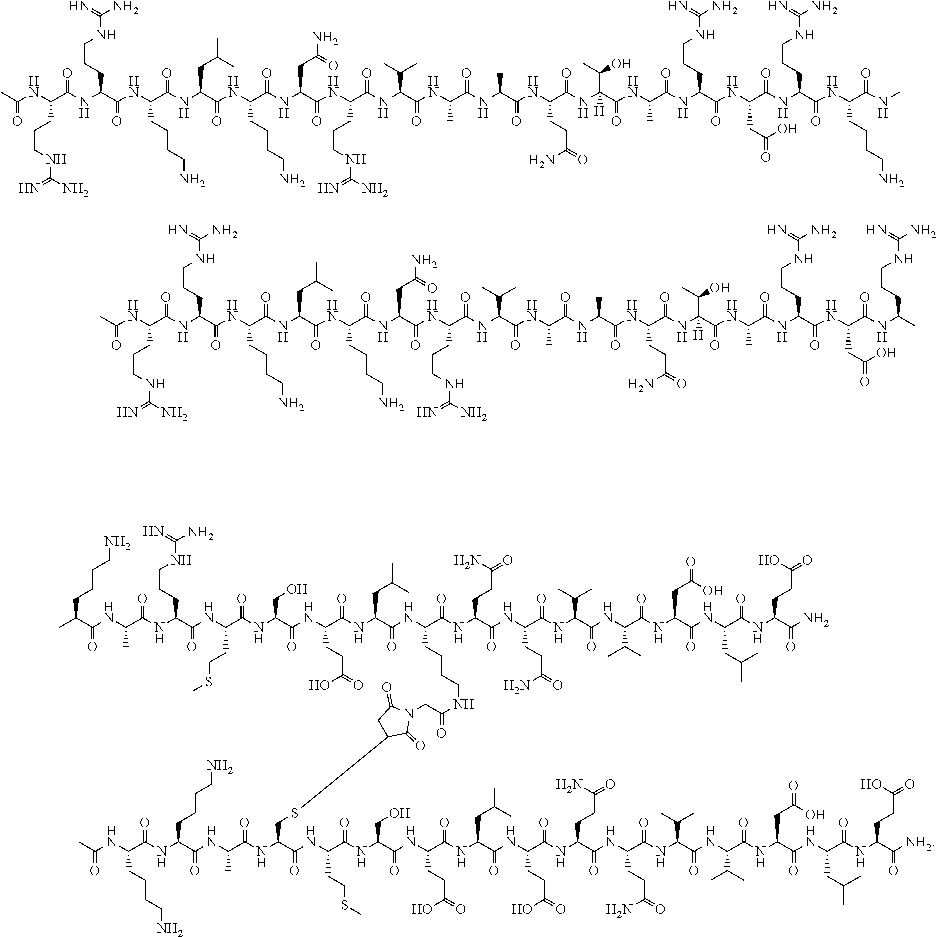

77. An engineered DNA-binding dimer having formula:

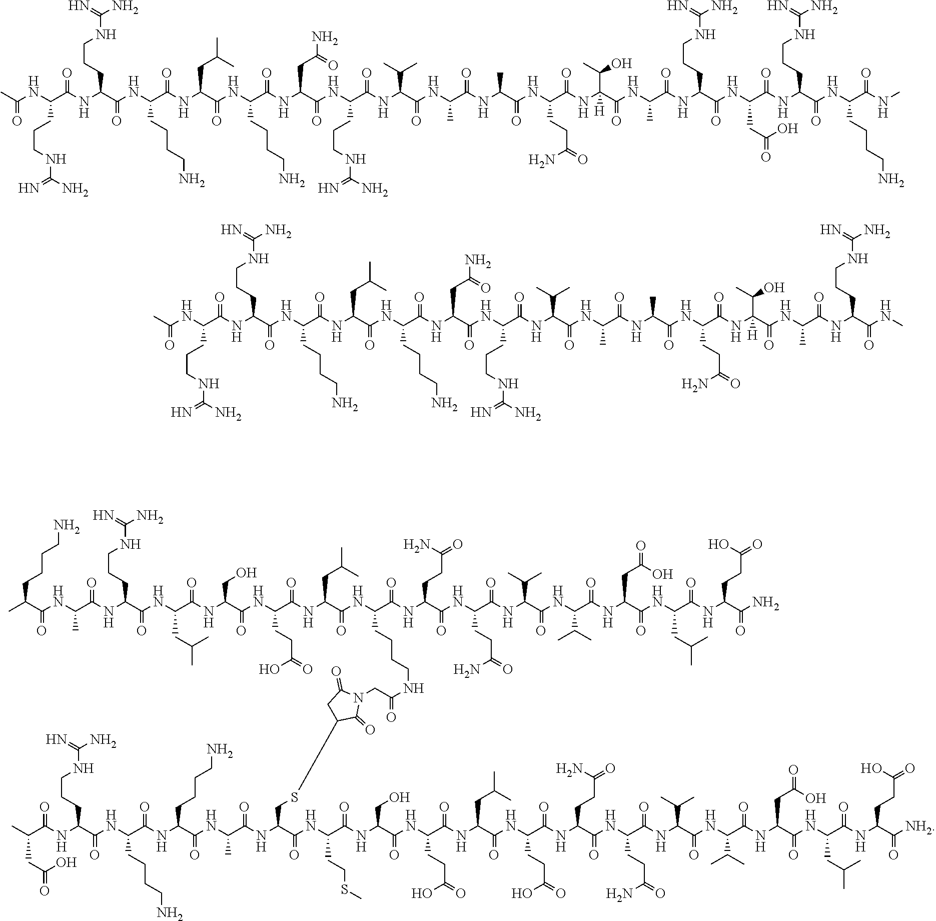

78. An engineered DNA-binding dimer having formula:

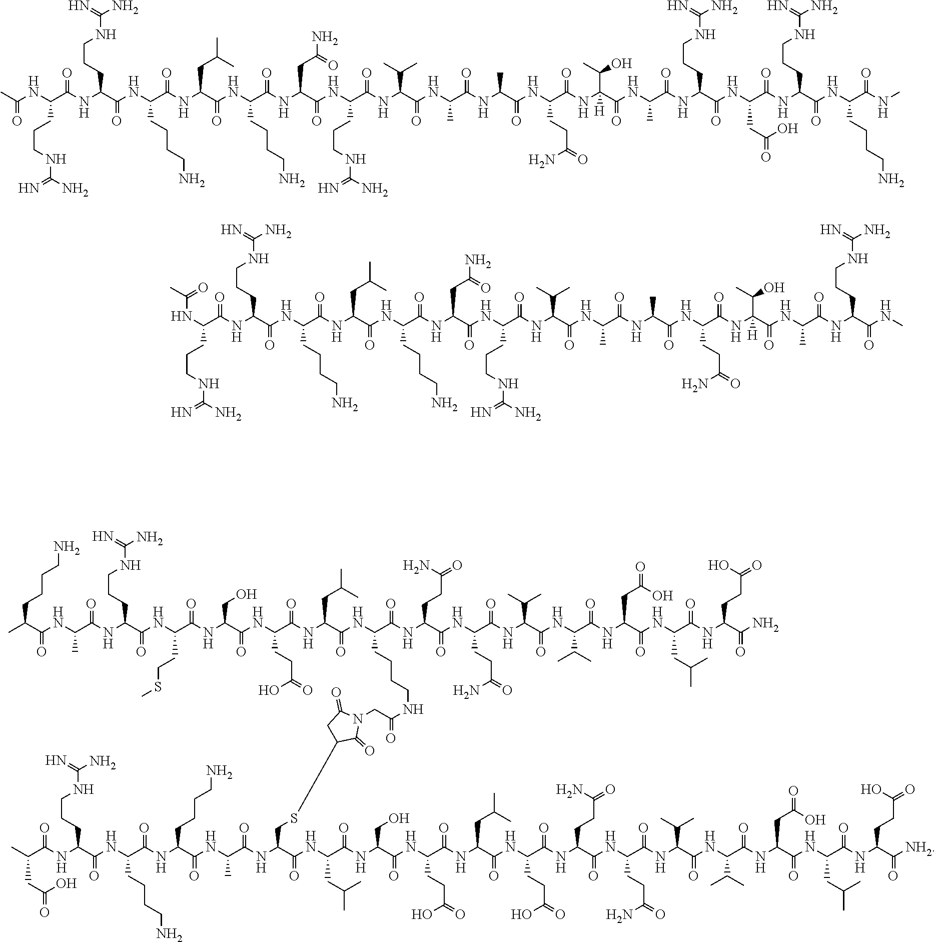

79. An engineered DNA-binding dimer having formula:

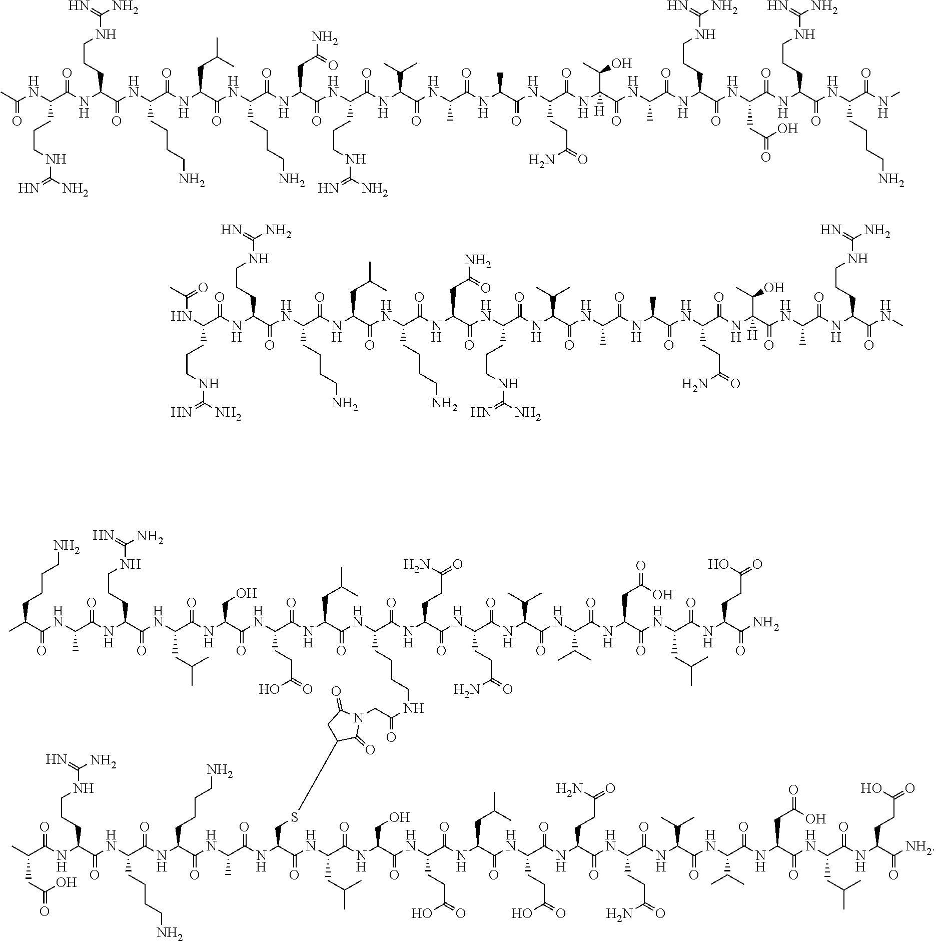

80. An engineered DNA-binding dimer having formula:

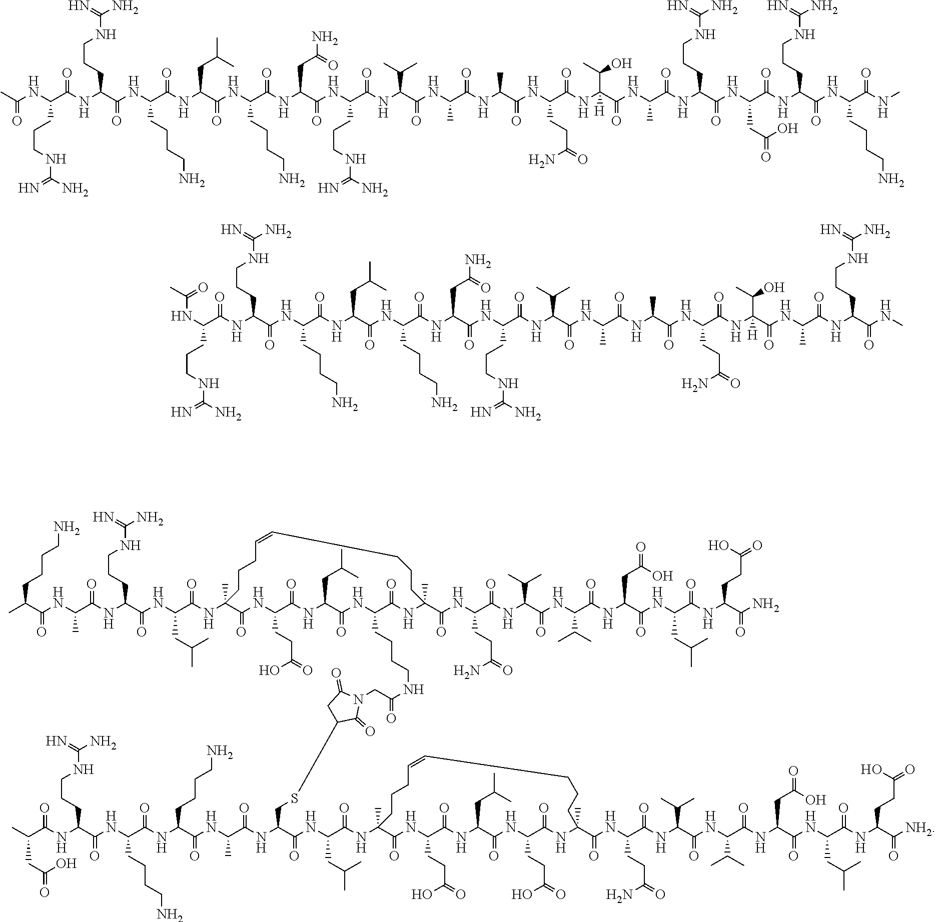

81. An engineered DNA-binding dimer having formula:

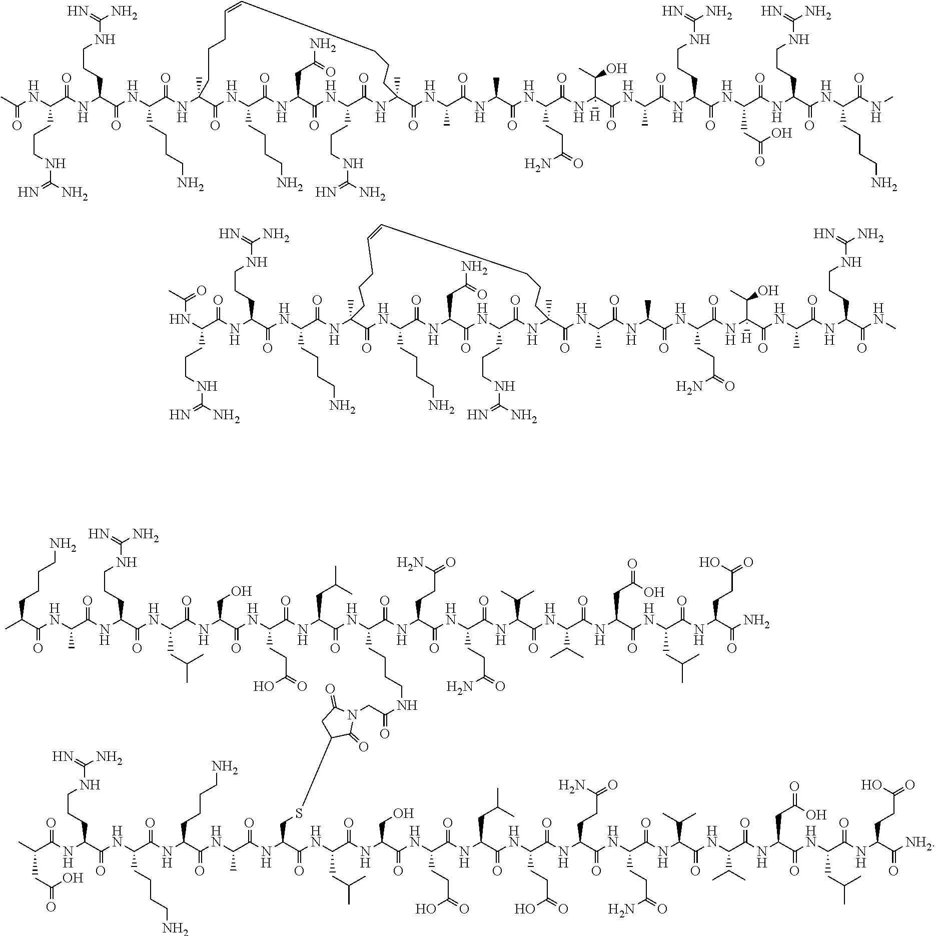

82. An engineered DNA-binding dimer having formula:

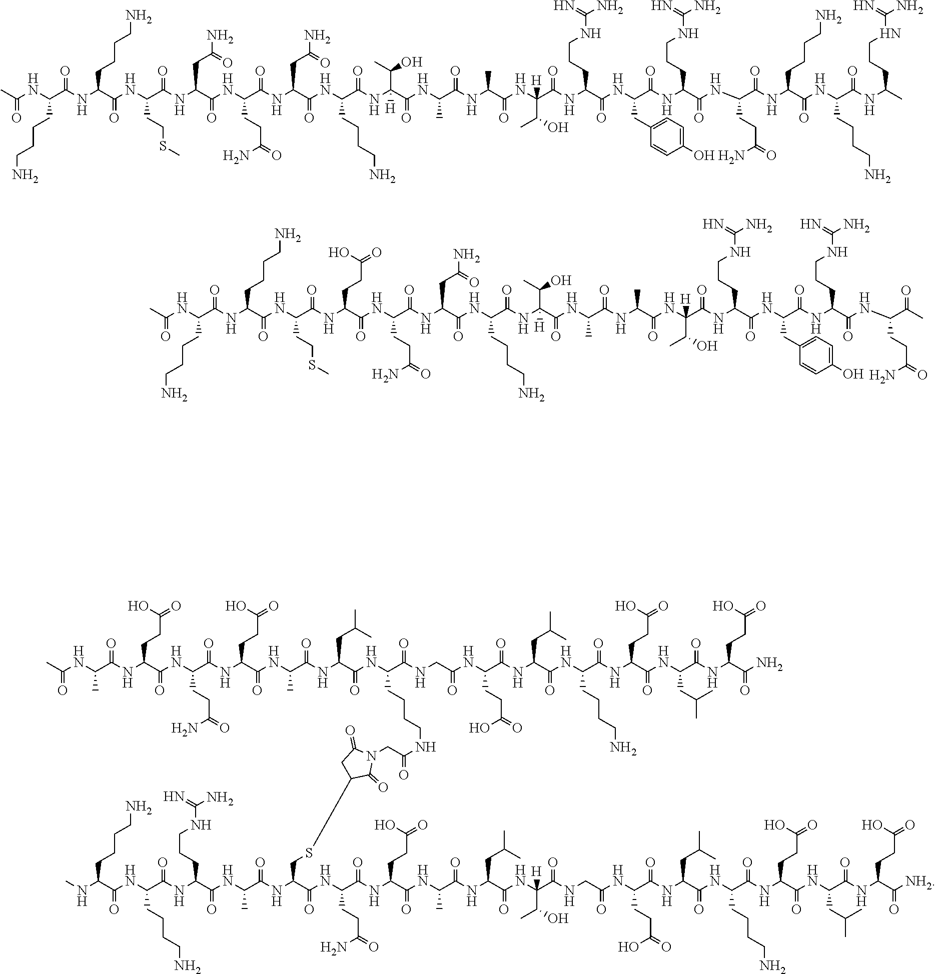

83. An engineered DNA-binding dimer having formula:

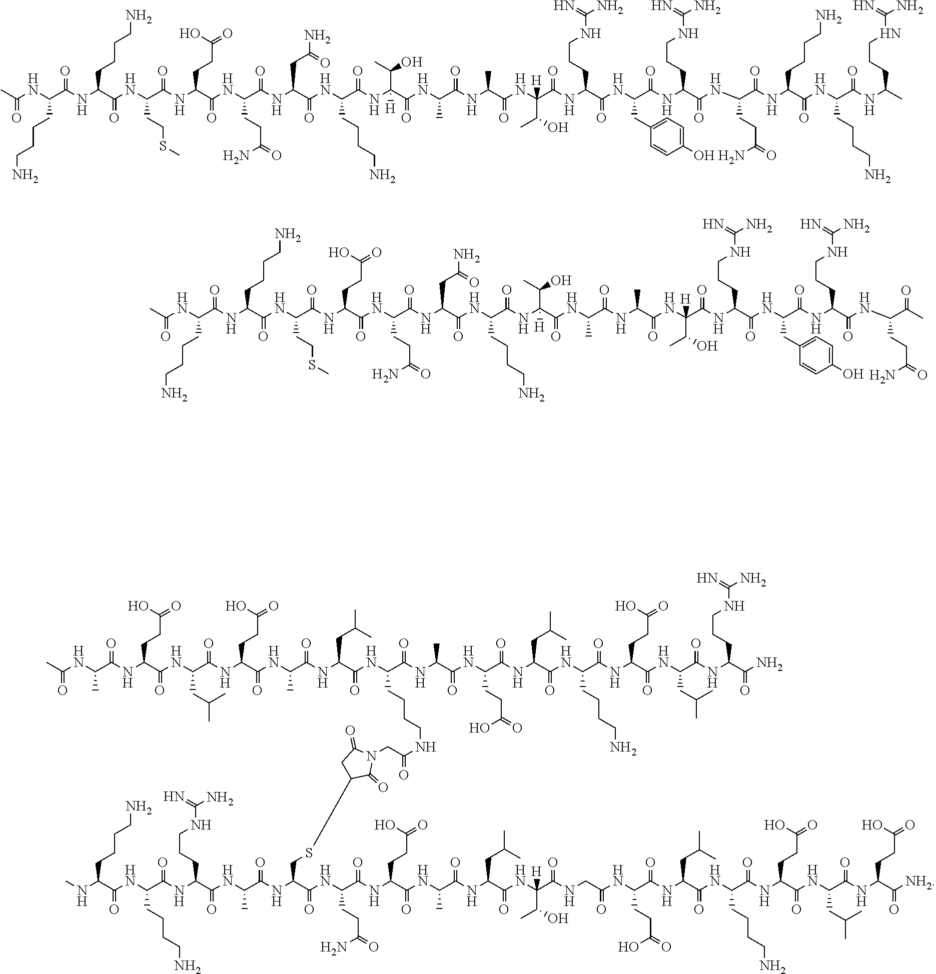

84. An engineered DNA-binding dimer having formula:

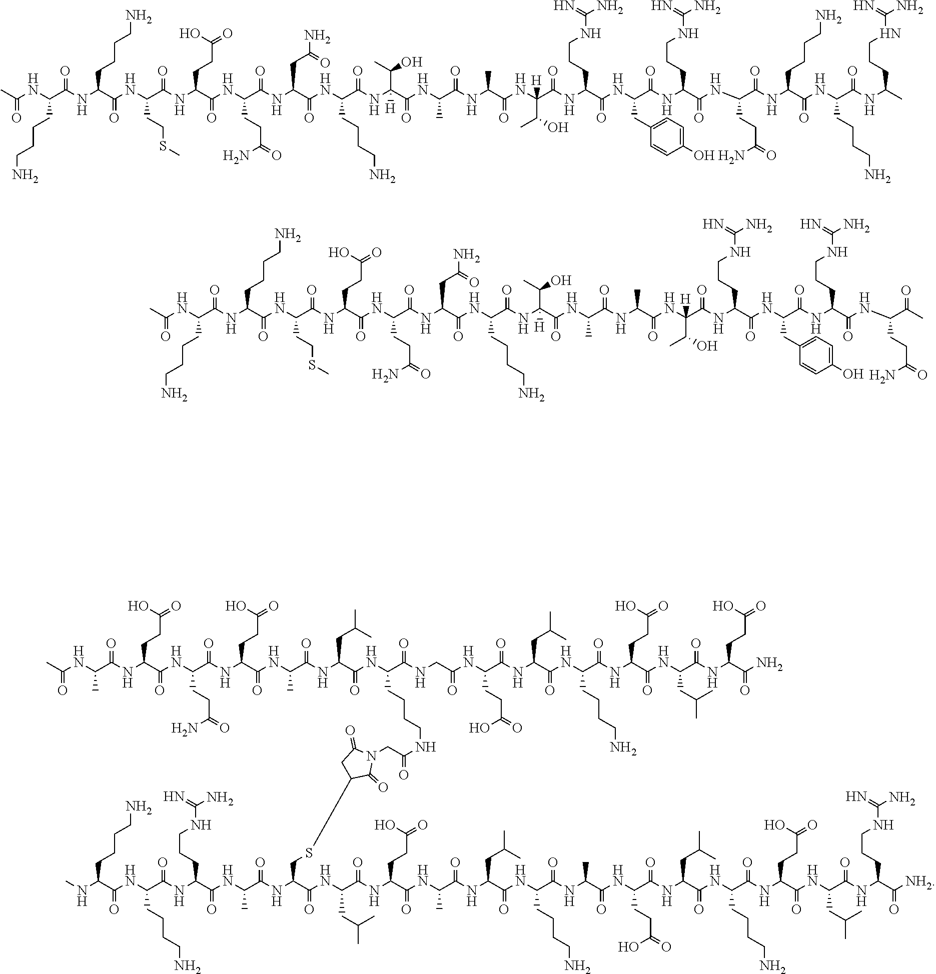

85. An engineered DNA-binding dimer having formula:

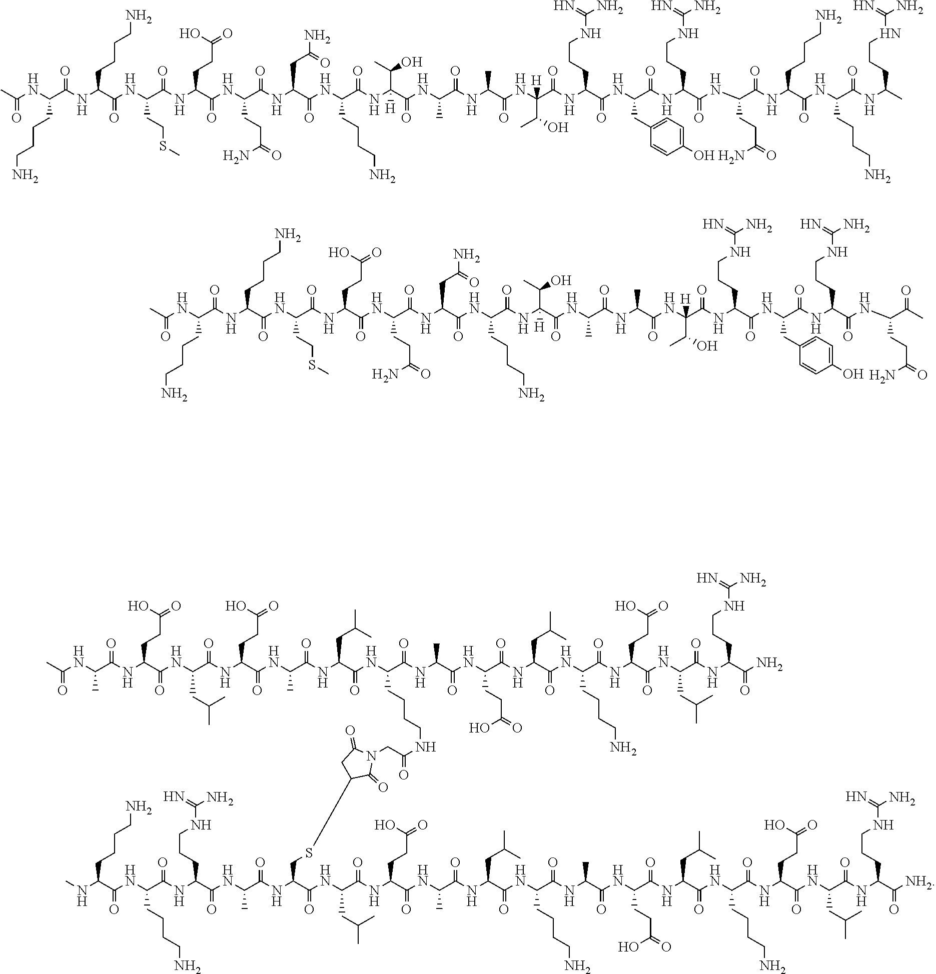

86. An engineered DNA-binding dimer having formula:

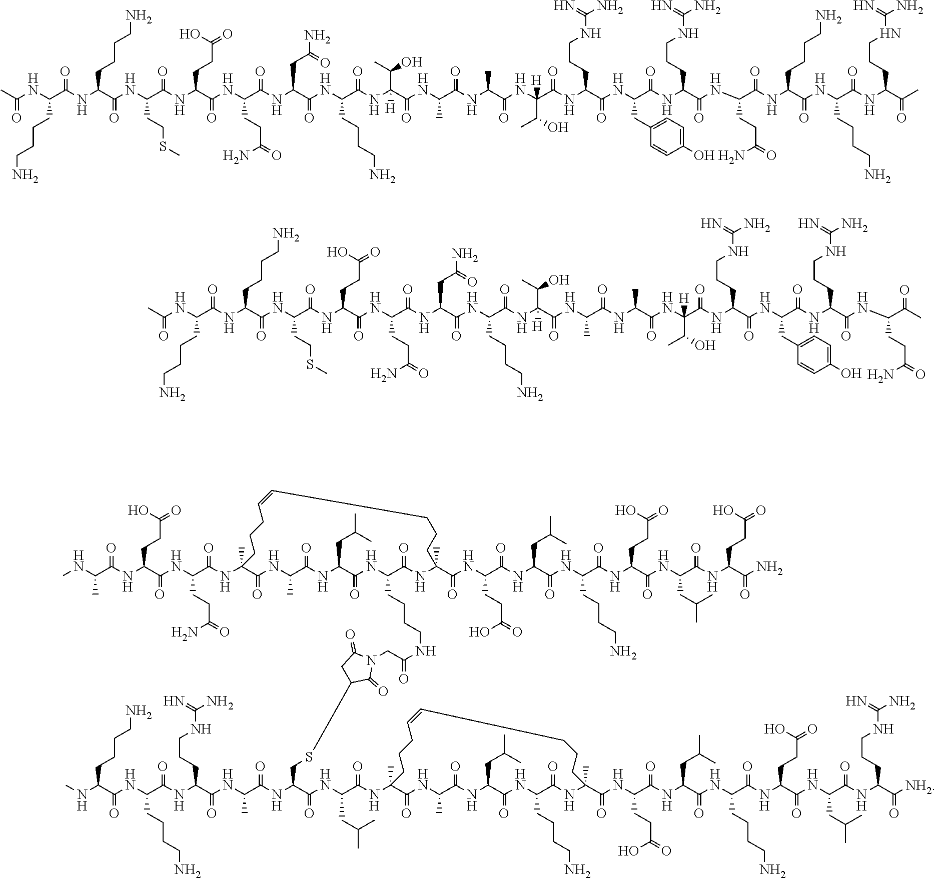

87. An engineered DNA-binding dimer having formula:

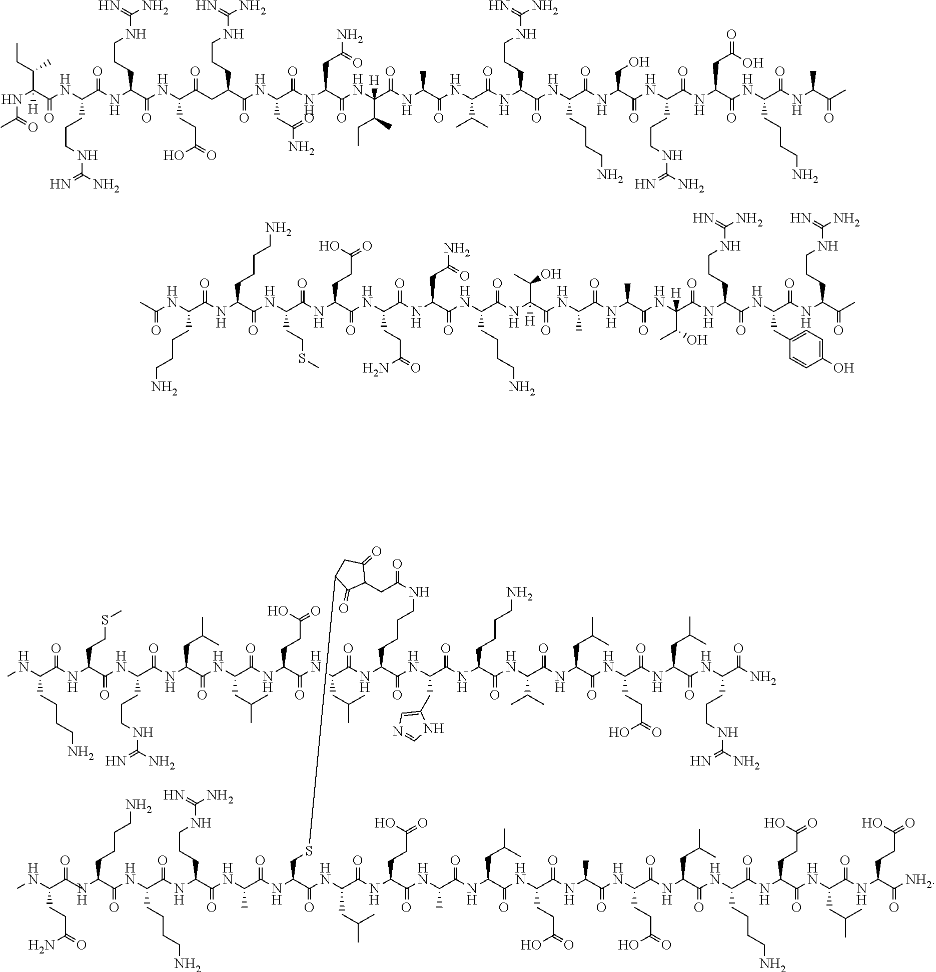

88. An engineered DNA-binding dimer having formula:

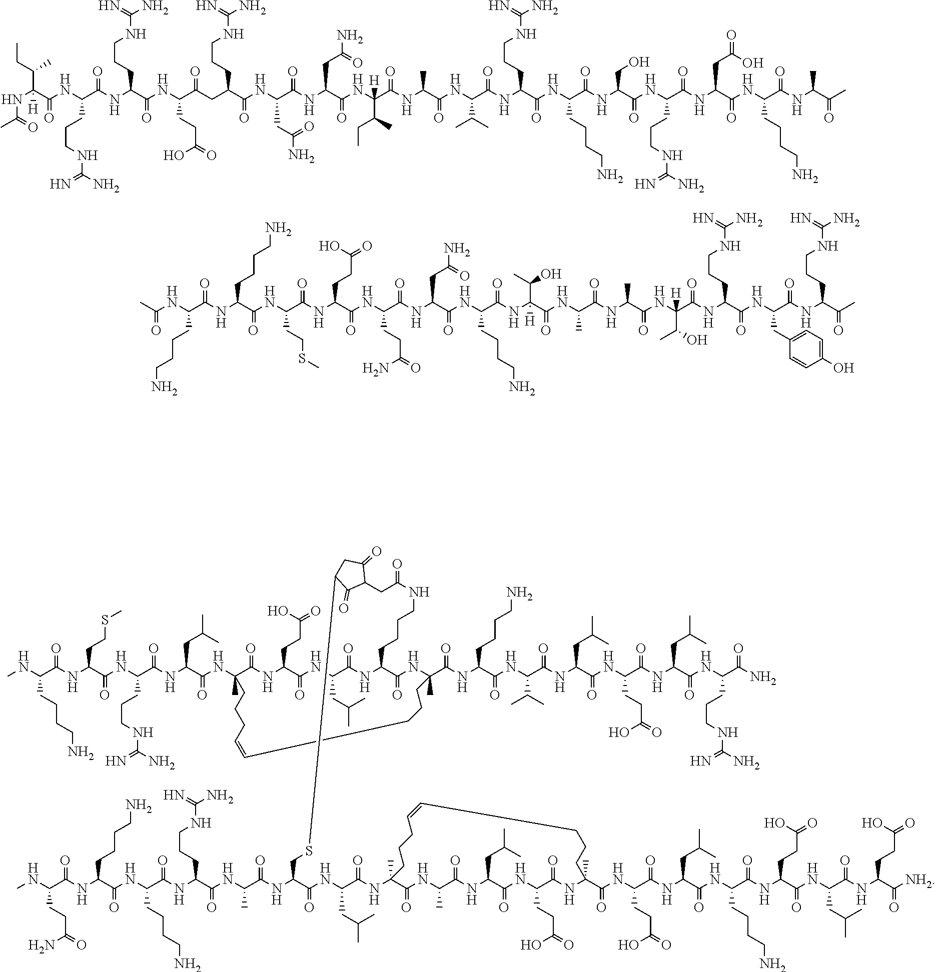

89. An engineered DNA-binding dimer having formula:

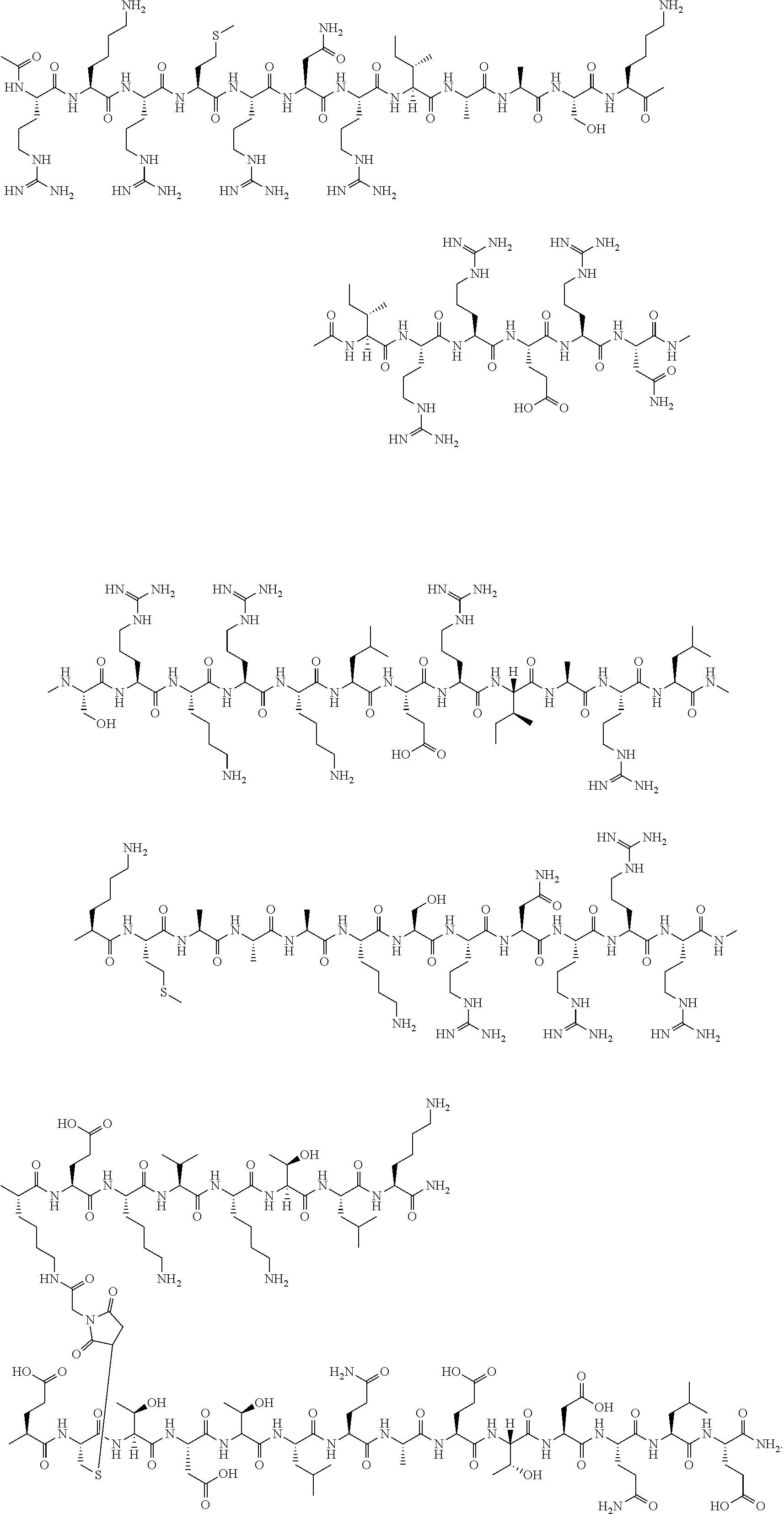

90. An engineered DNA-binding dimer having formula:

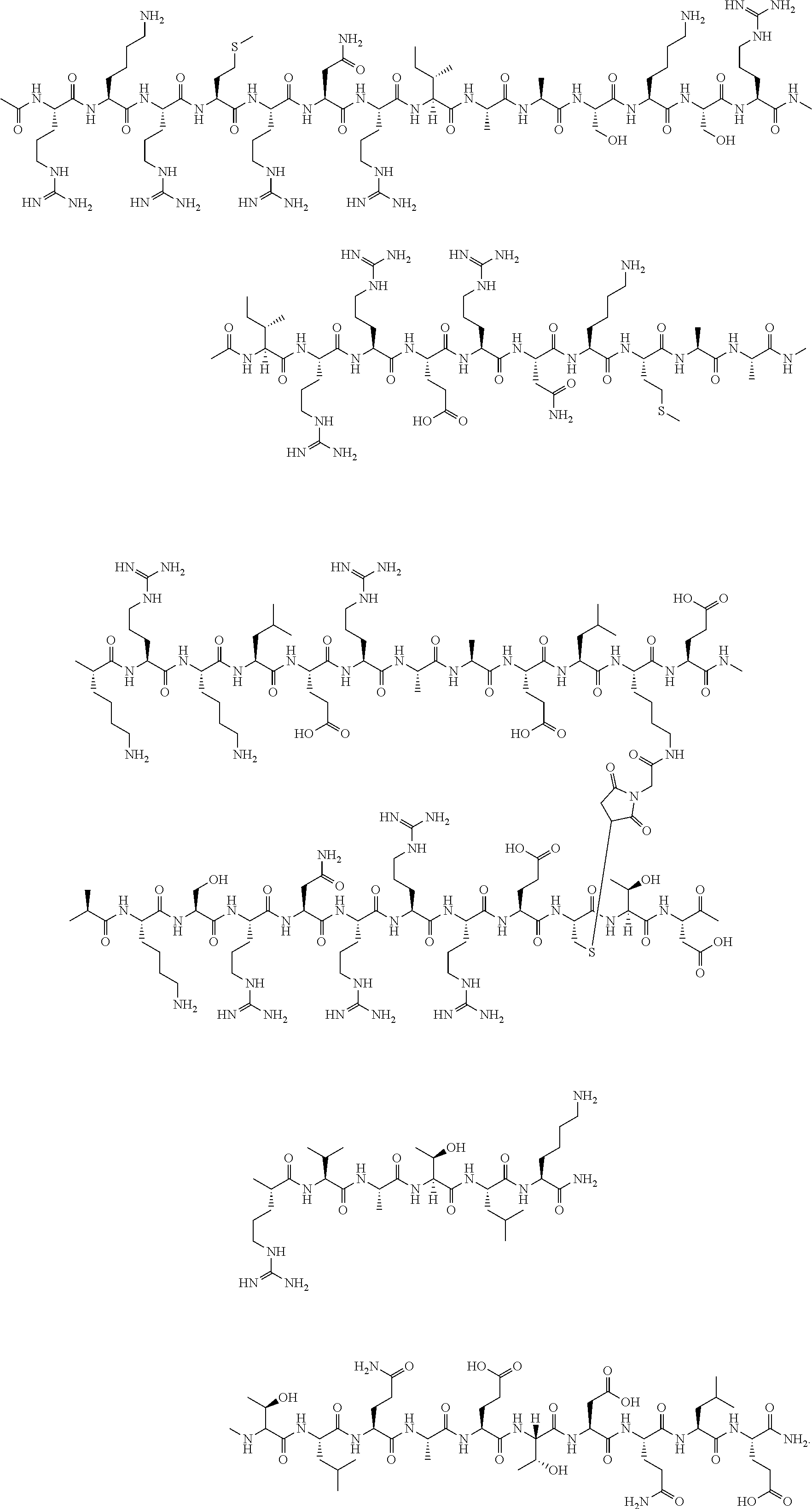

91. An engineered DNA-binding dimer having formula:

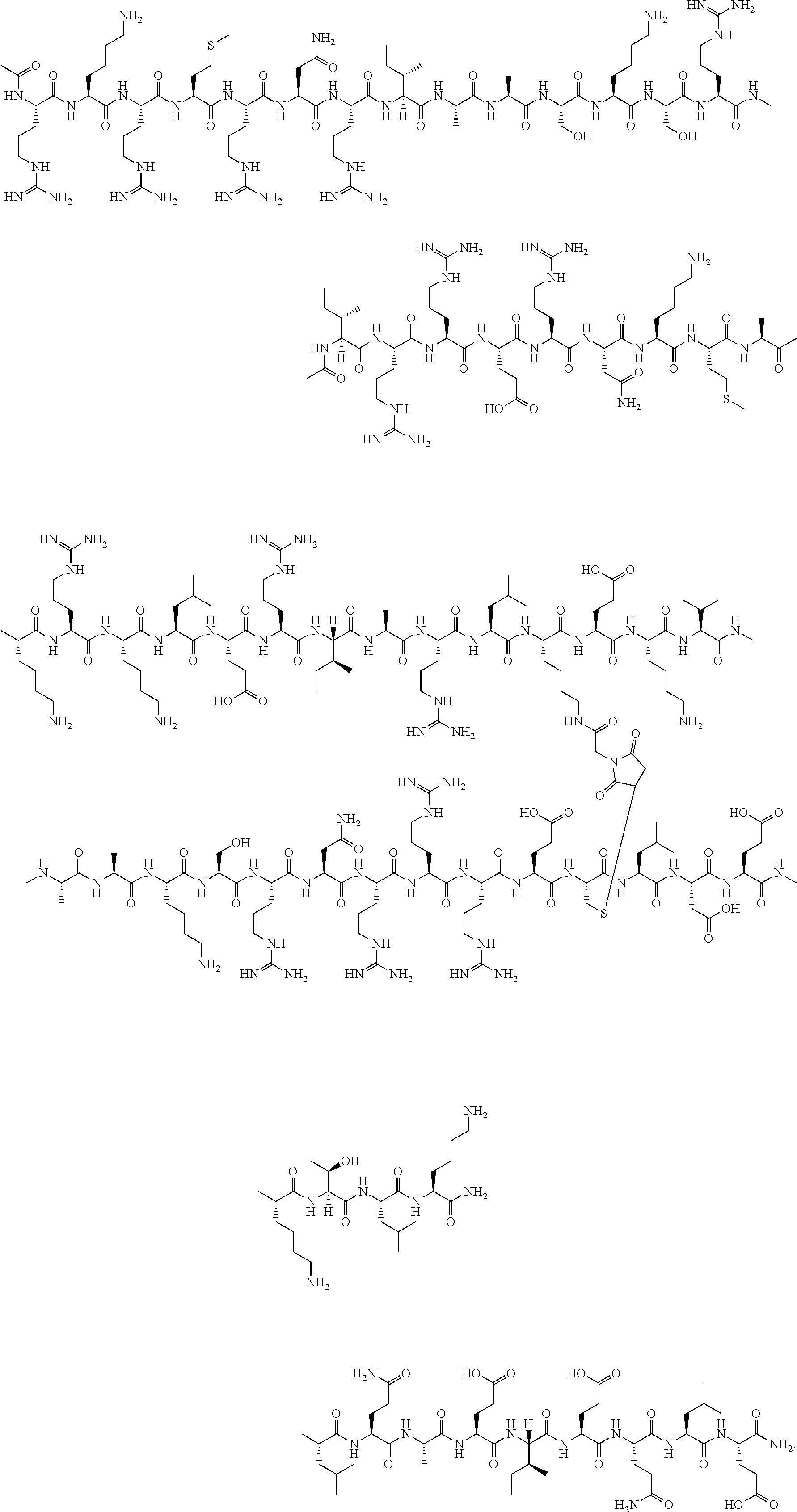

92. An engineered DNA-binding dimer having formula:

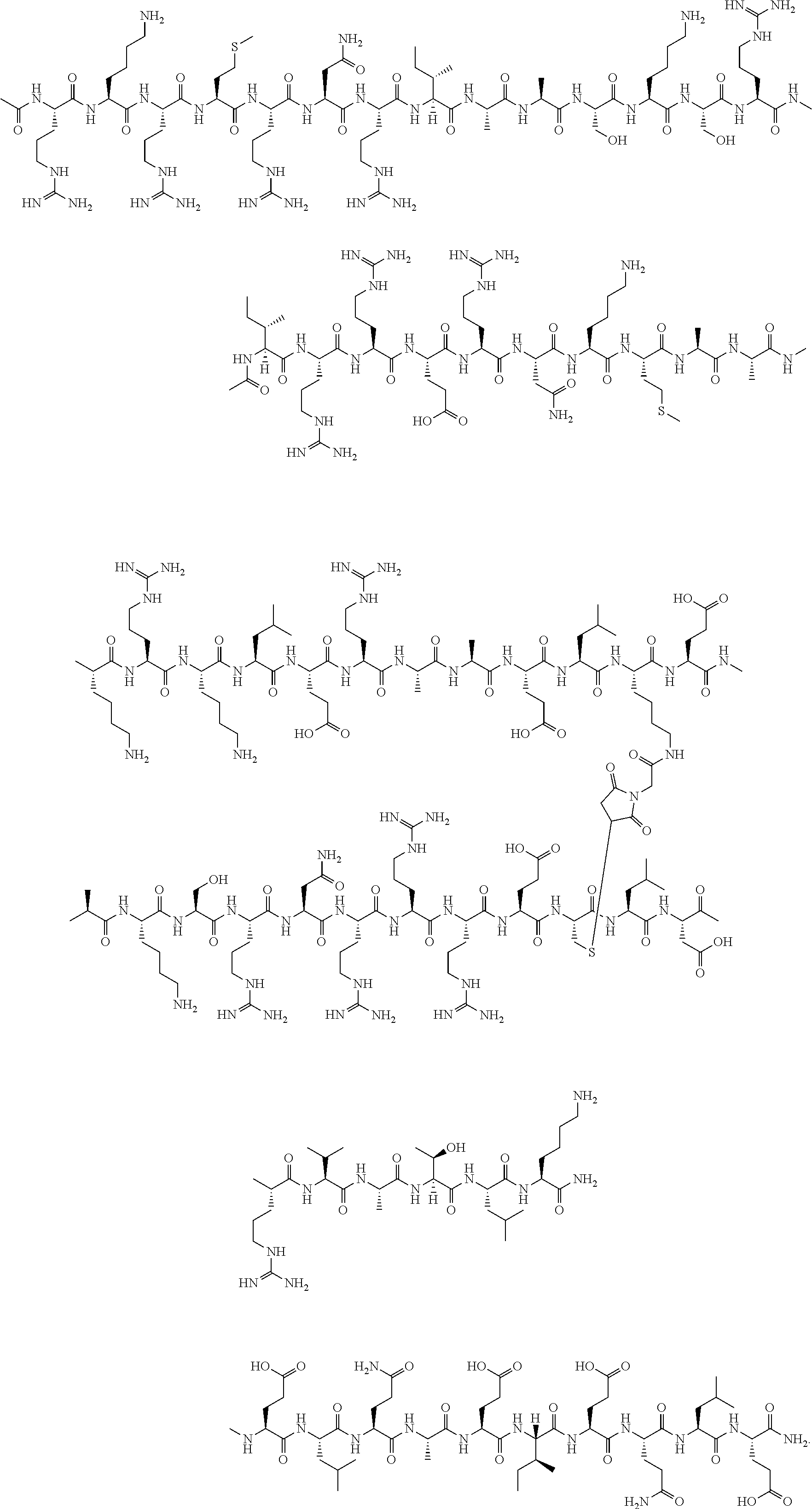

93. An engineered DNA-binding dimer having formula:

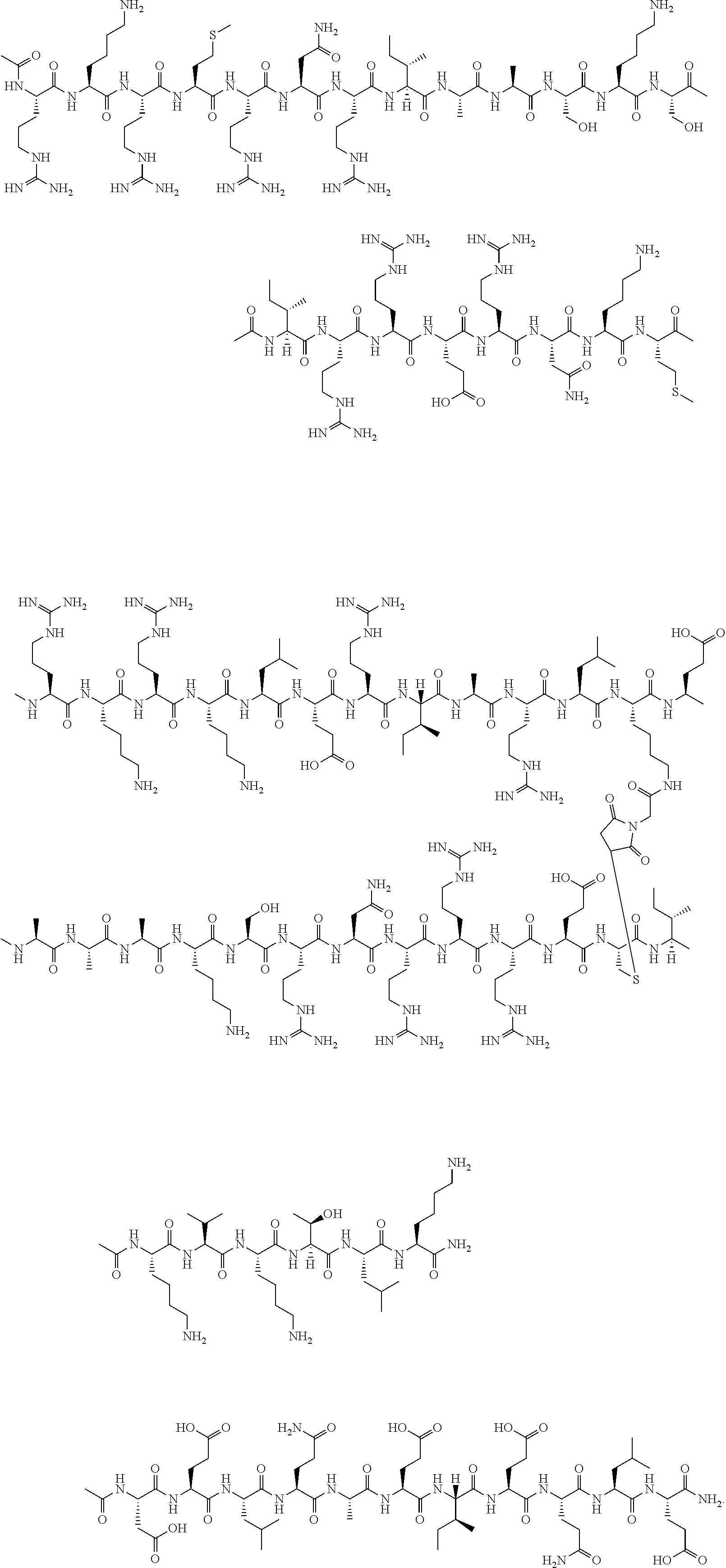

94. An engineered DNA-binding dimer having formula:

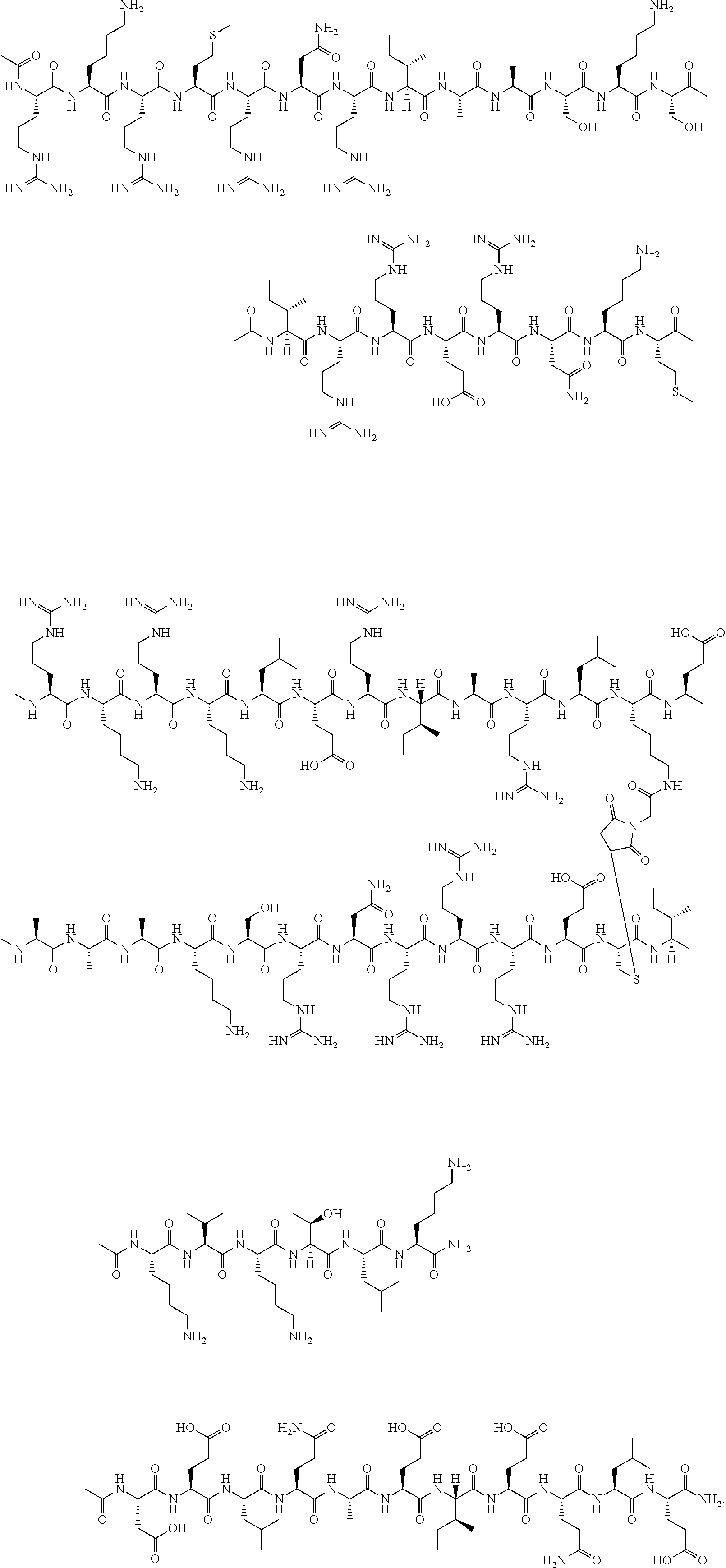

95. An engineered DNA-binding dimer having formula:

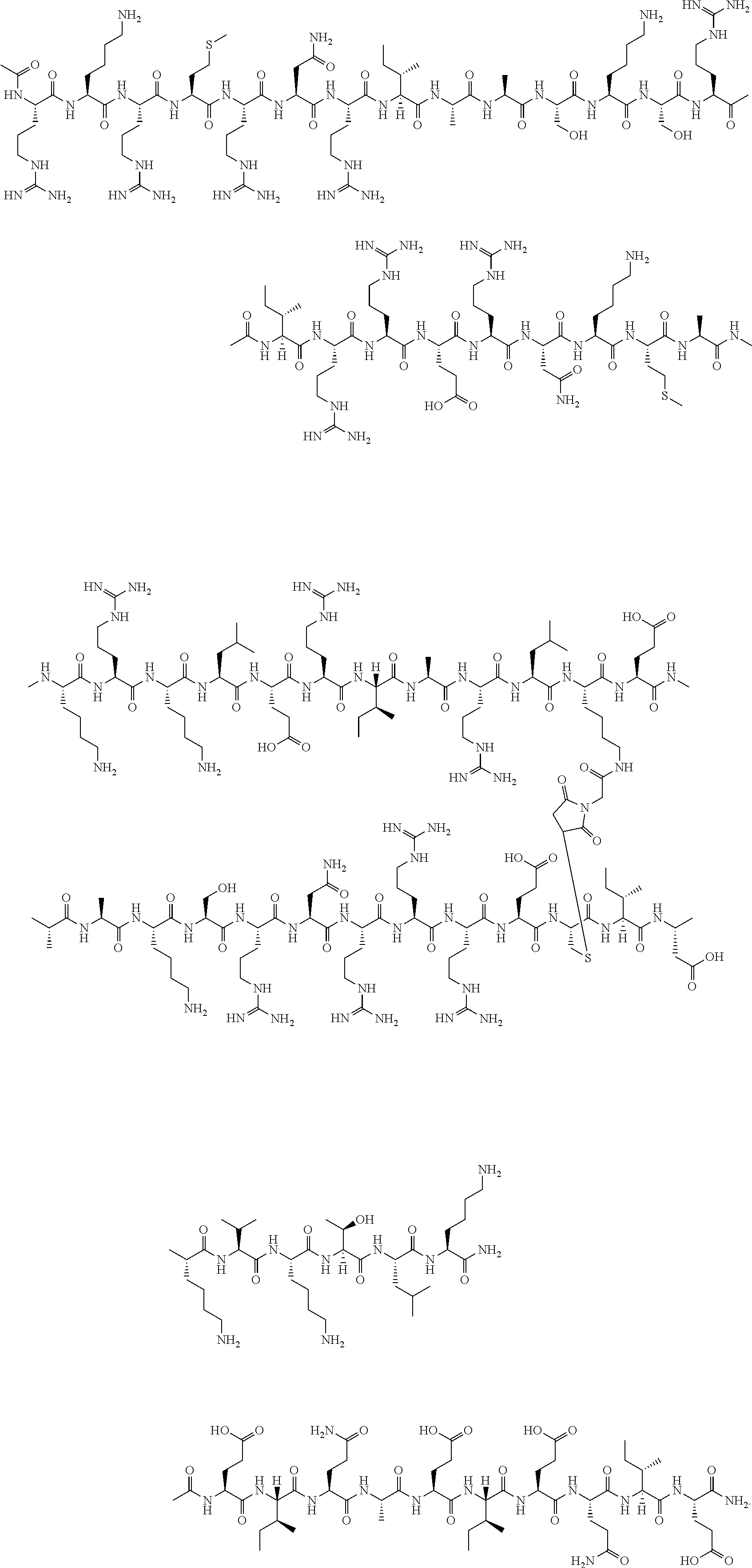

96. An engineered DNA-binding dimer having formula:

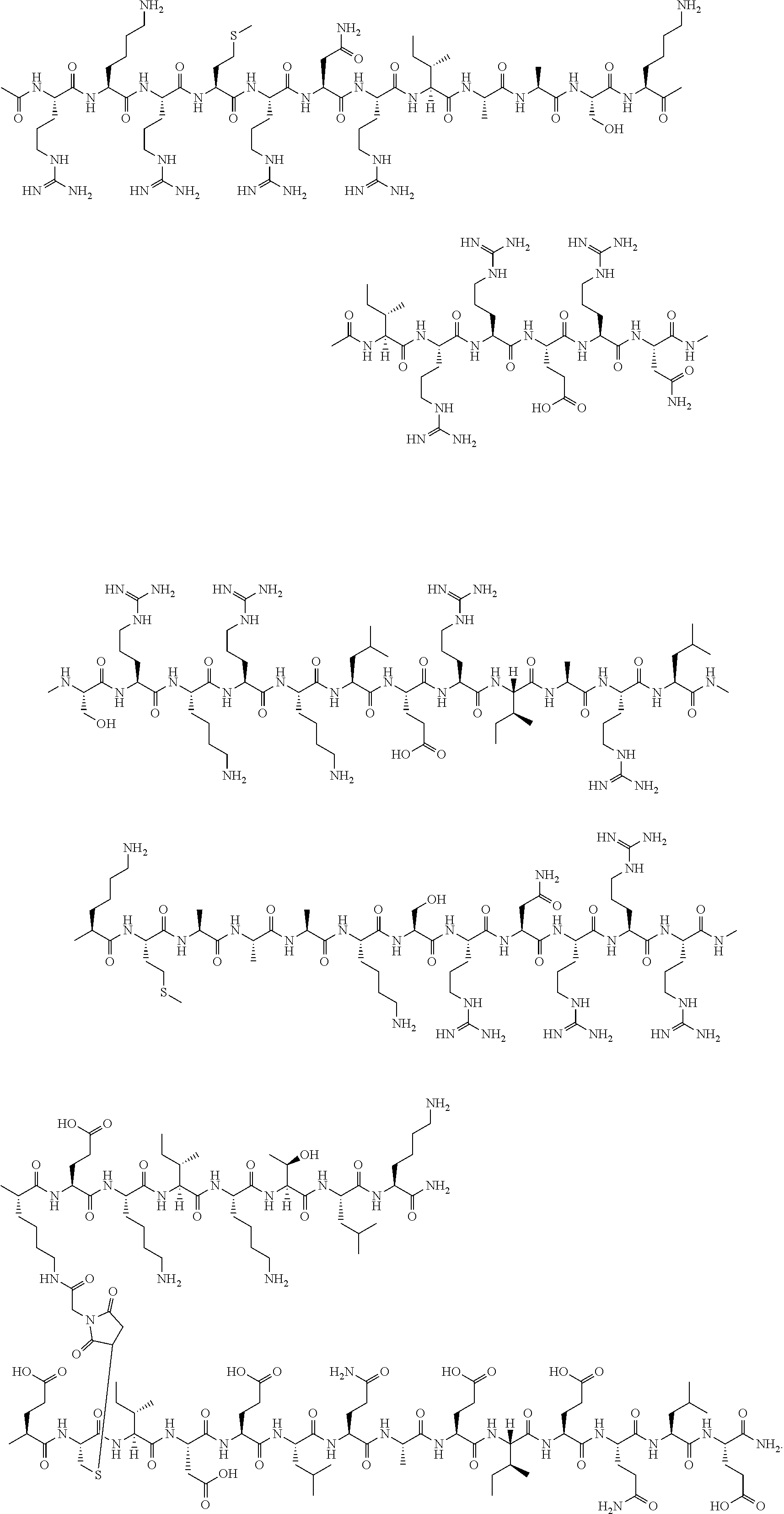

97. An engineered DNA-binding dimer having formula:

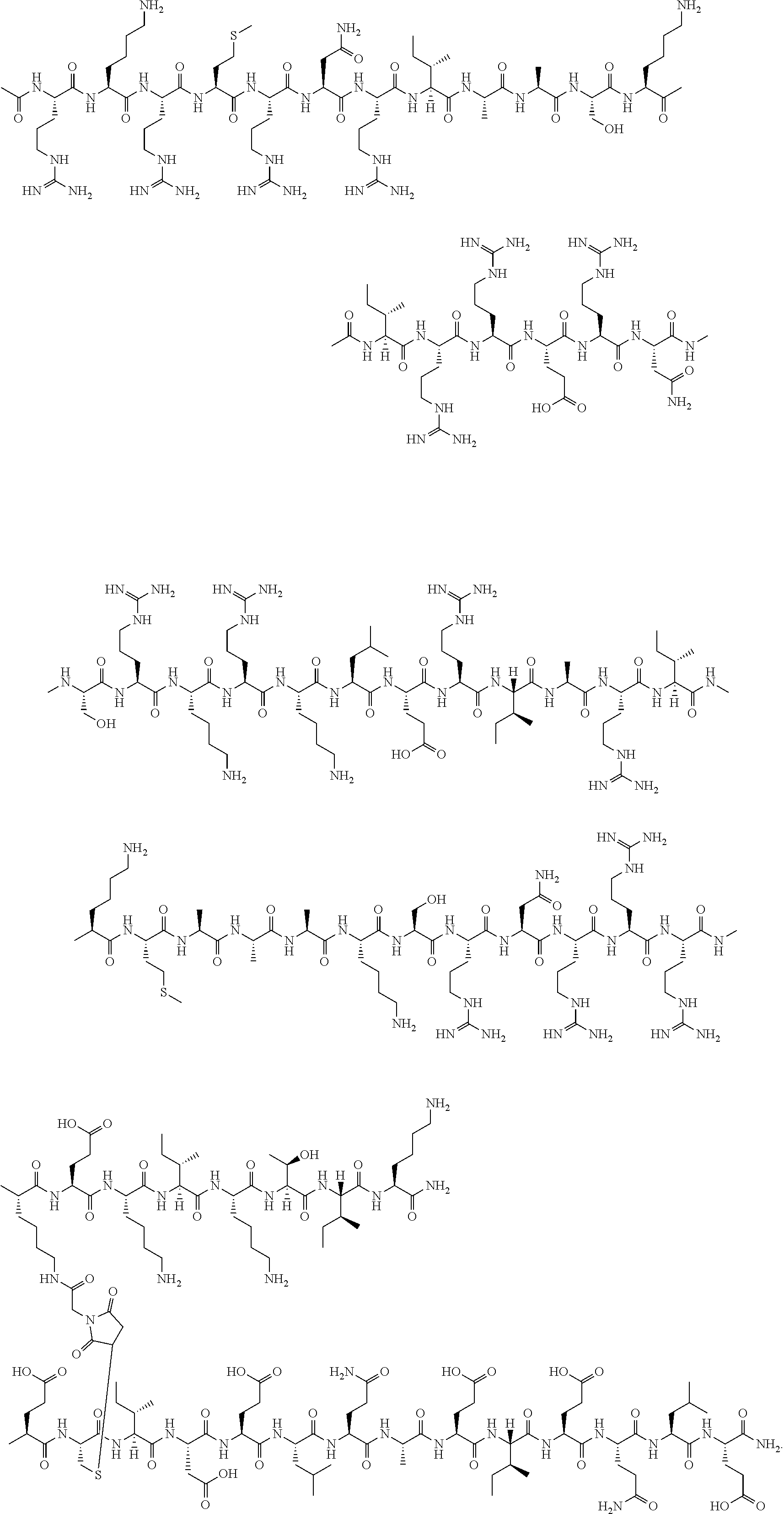

98. An engineered DNA-binding dimer having formula:

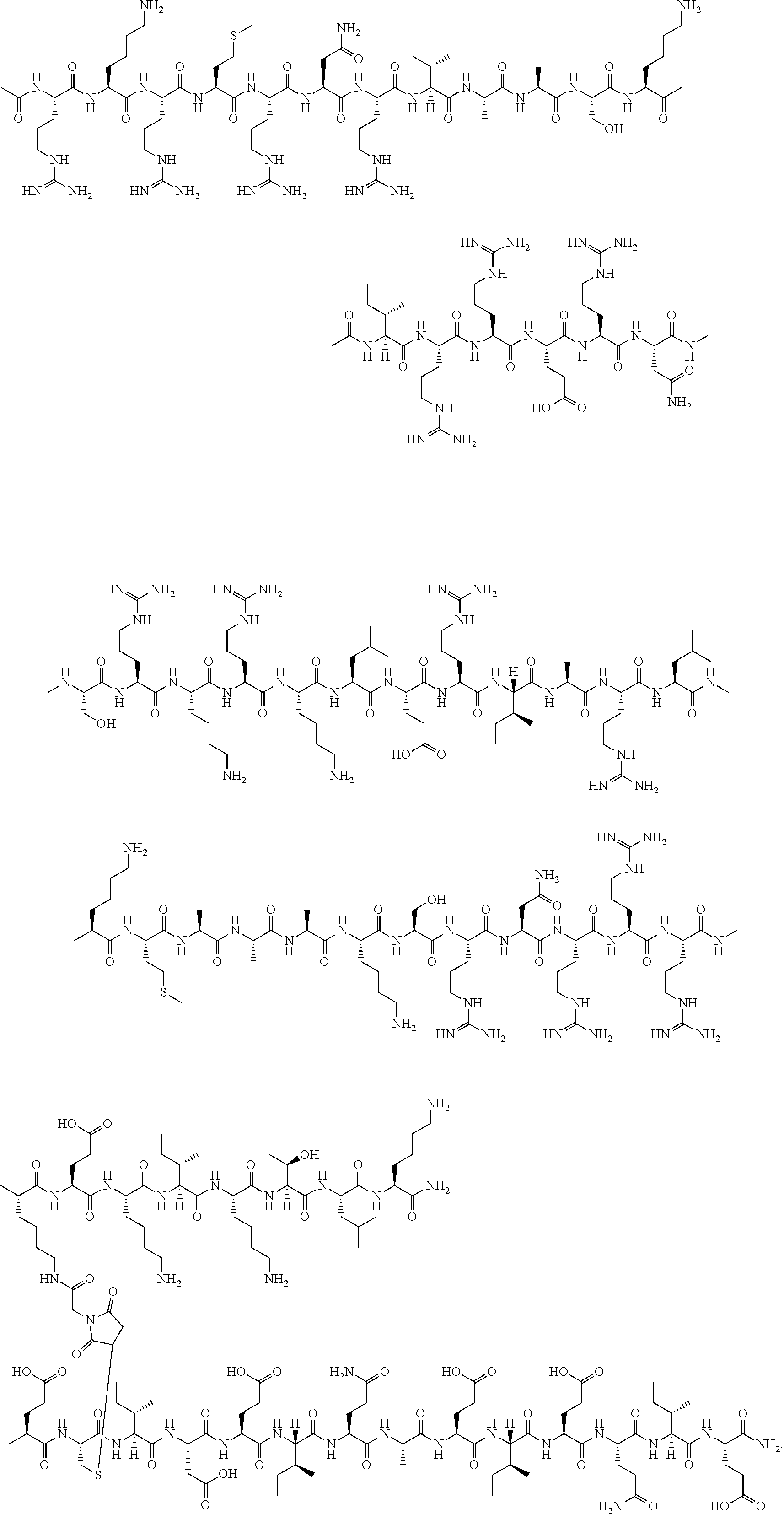

99. An engineered DNA-binding dimer having formula:

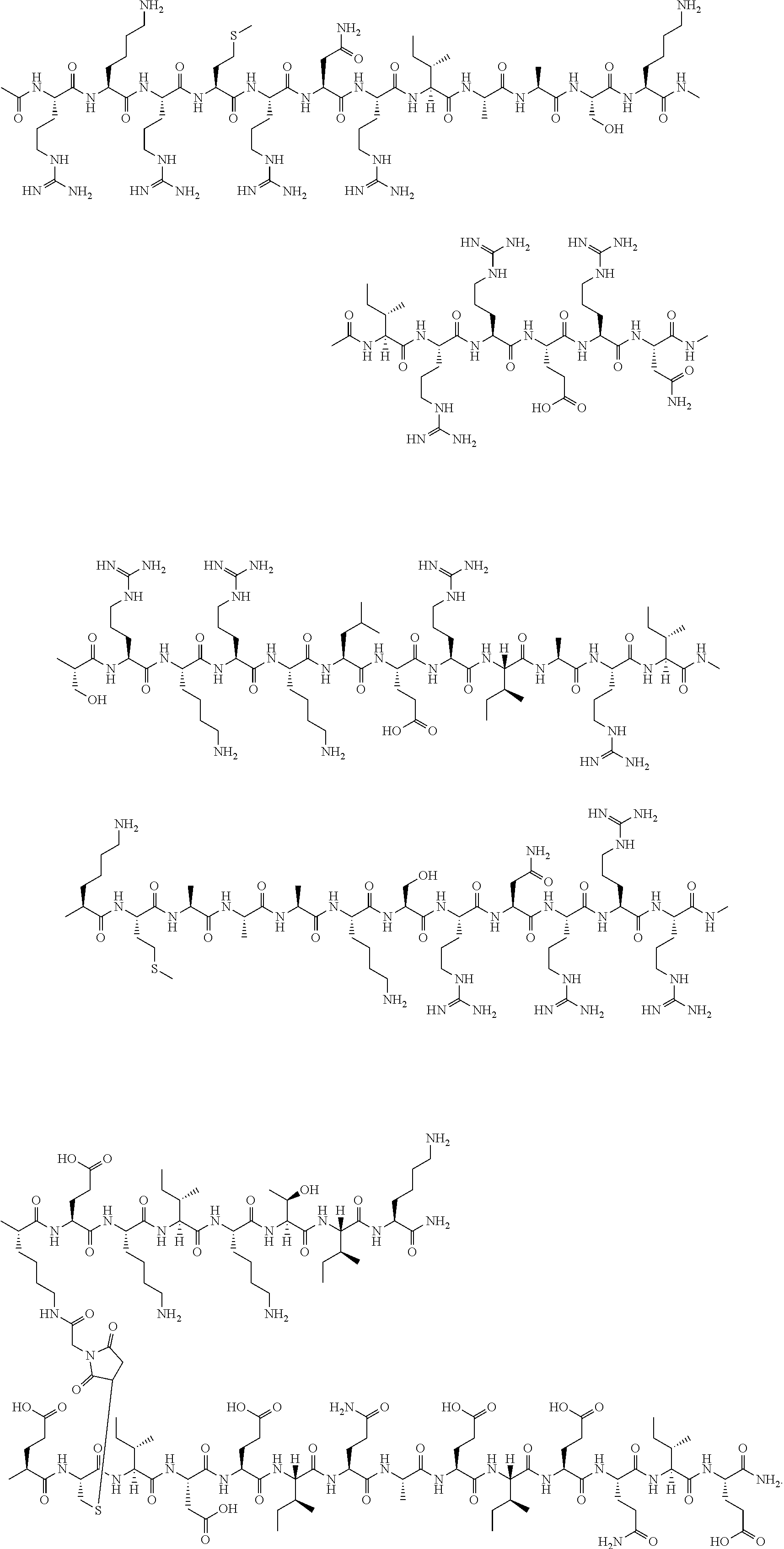

100. An engineered DNA-binding dimer having formula:

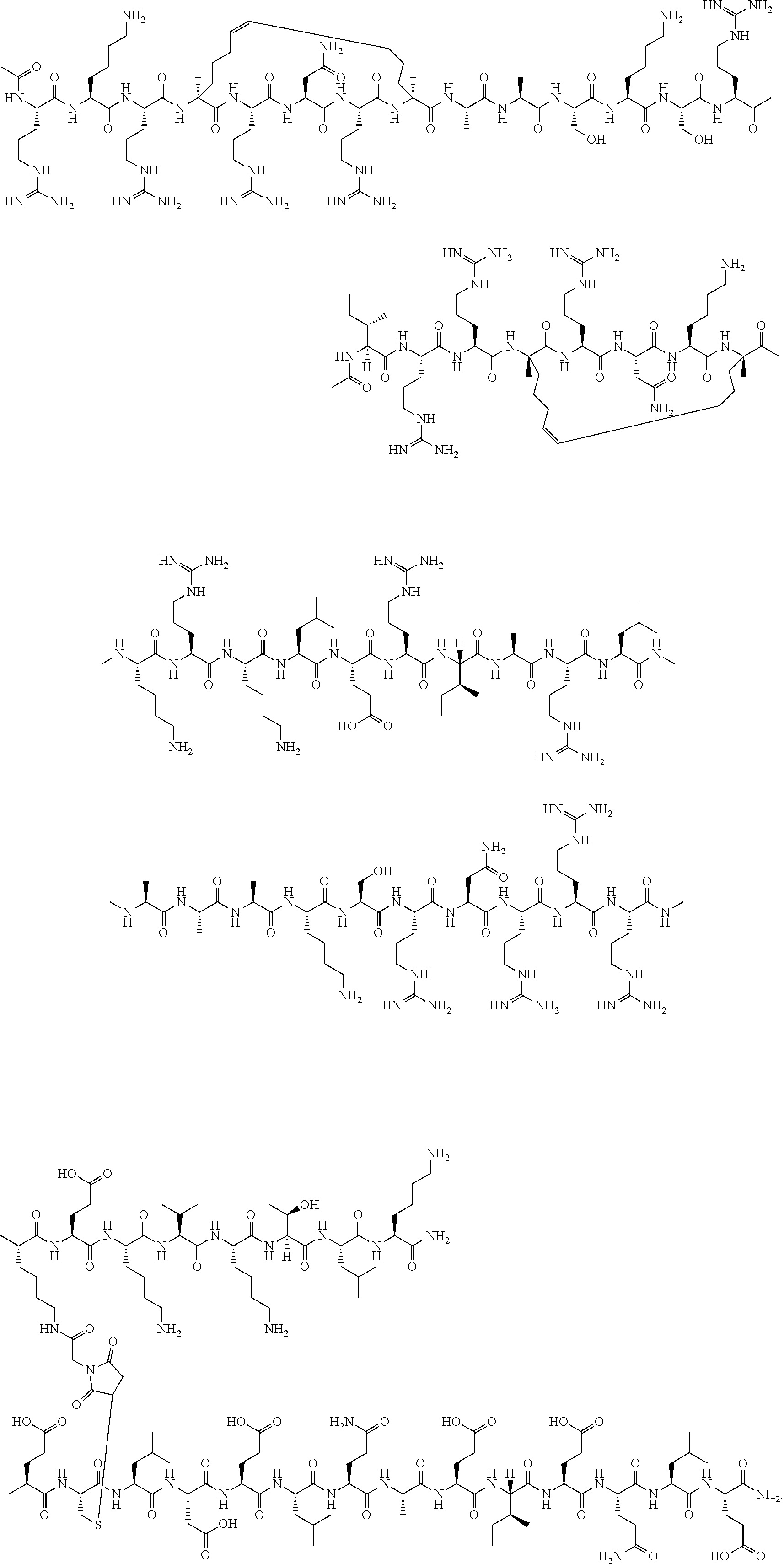

101. An engineered DNA-binding dimer having formula:

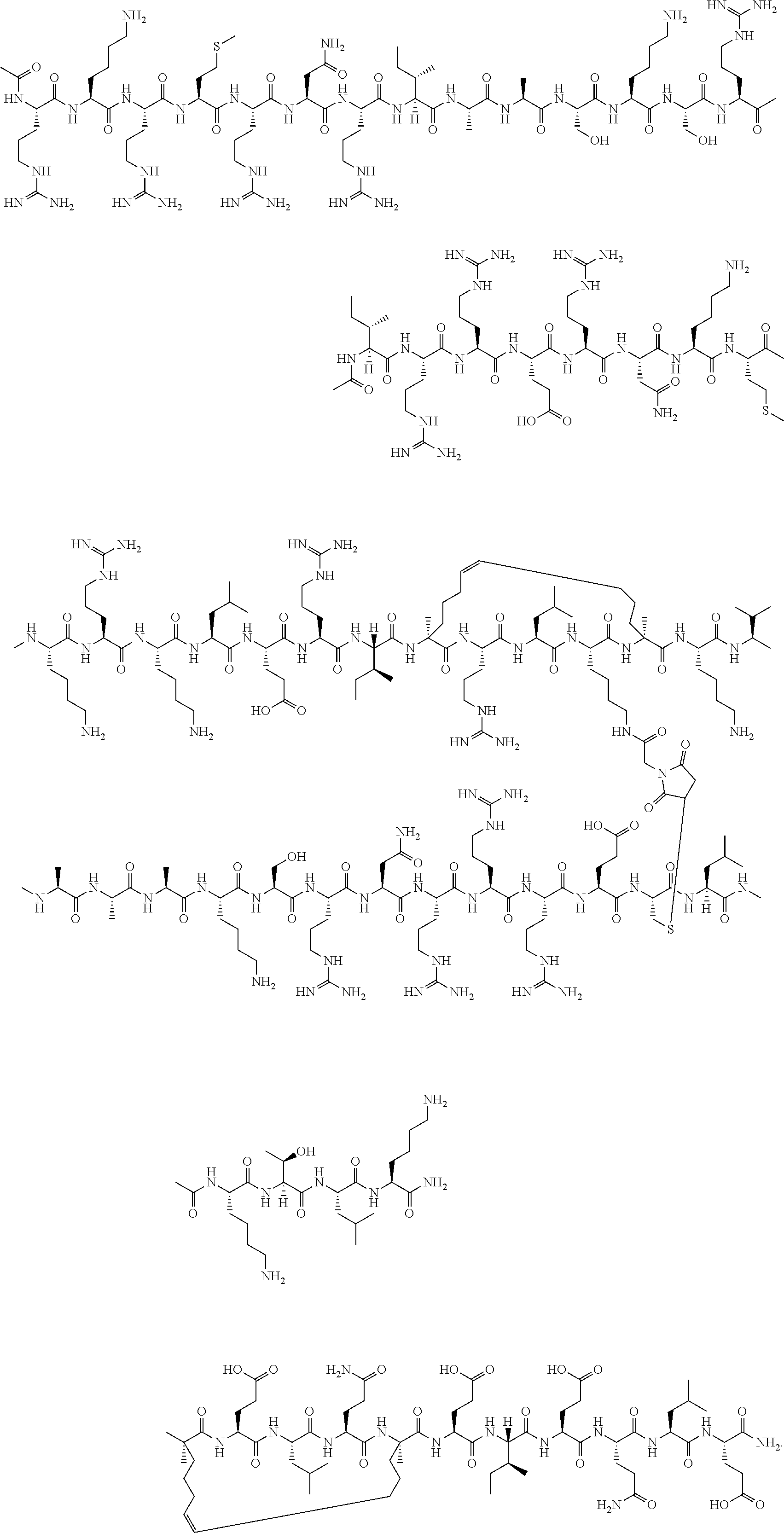

102. An engineered DNA-binding dimer having formula:

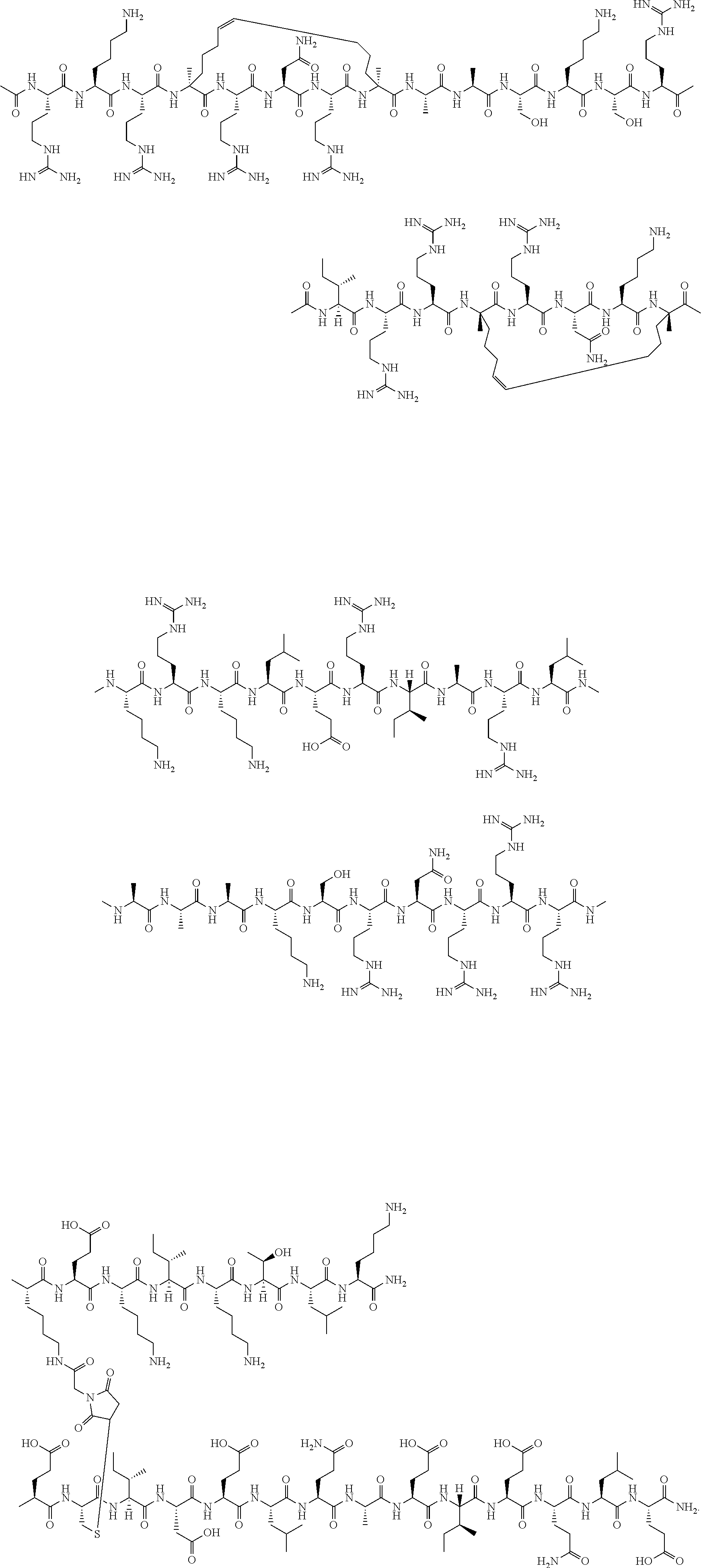

103. An engineered DNA-binding dimer having formula:

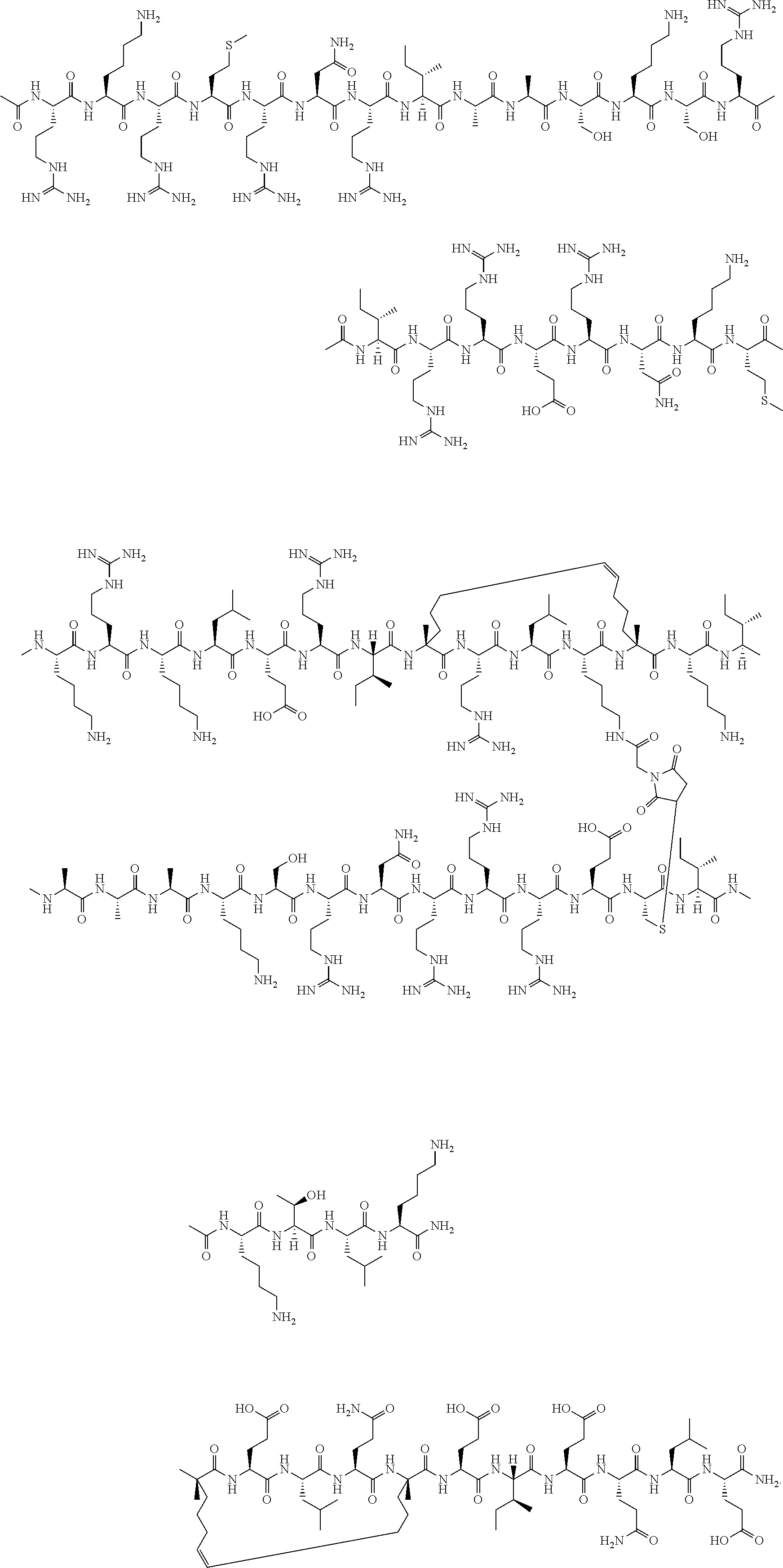

104. An engineered DNA-binding dimer having formula:

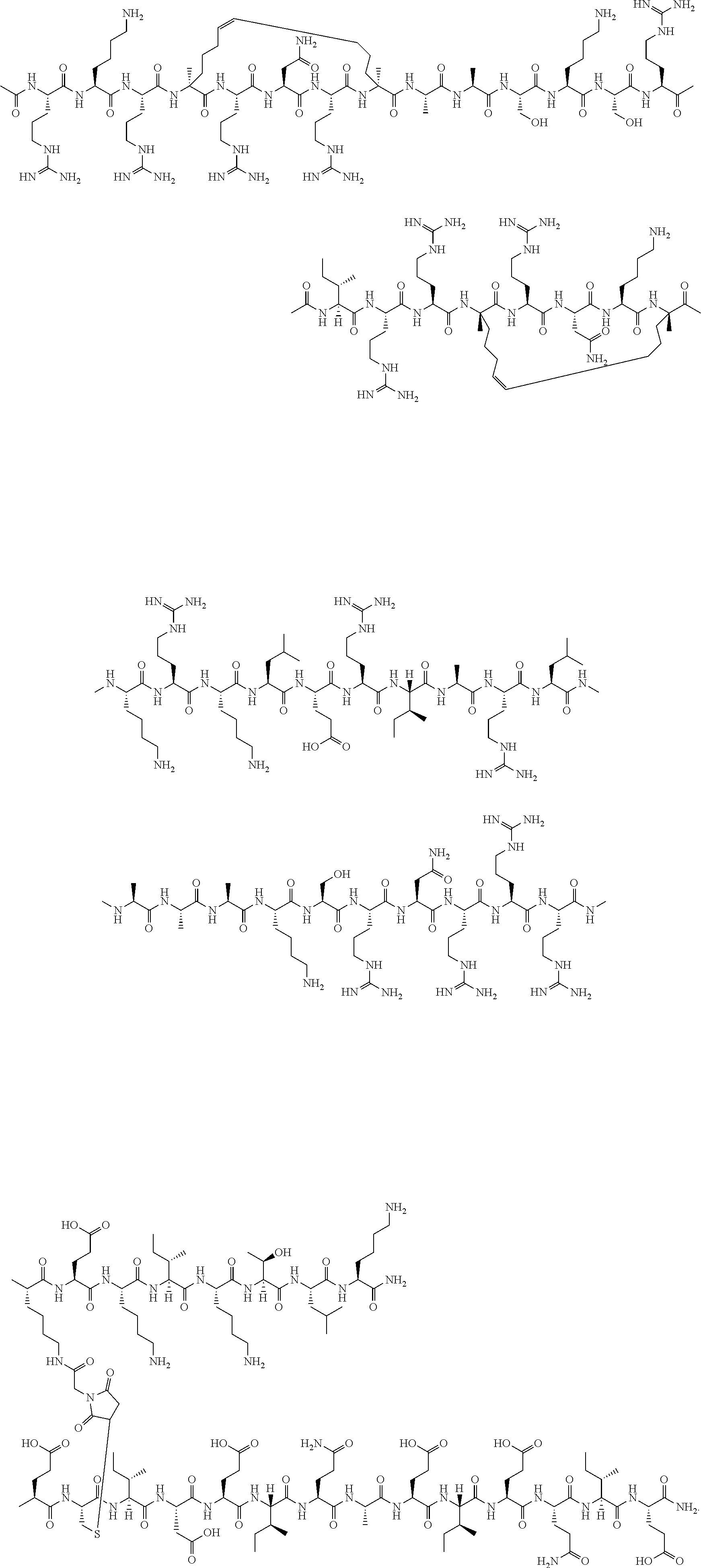

105. An engineered DNA-binding dimer having formula:

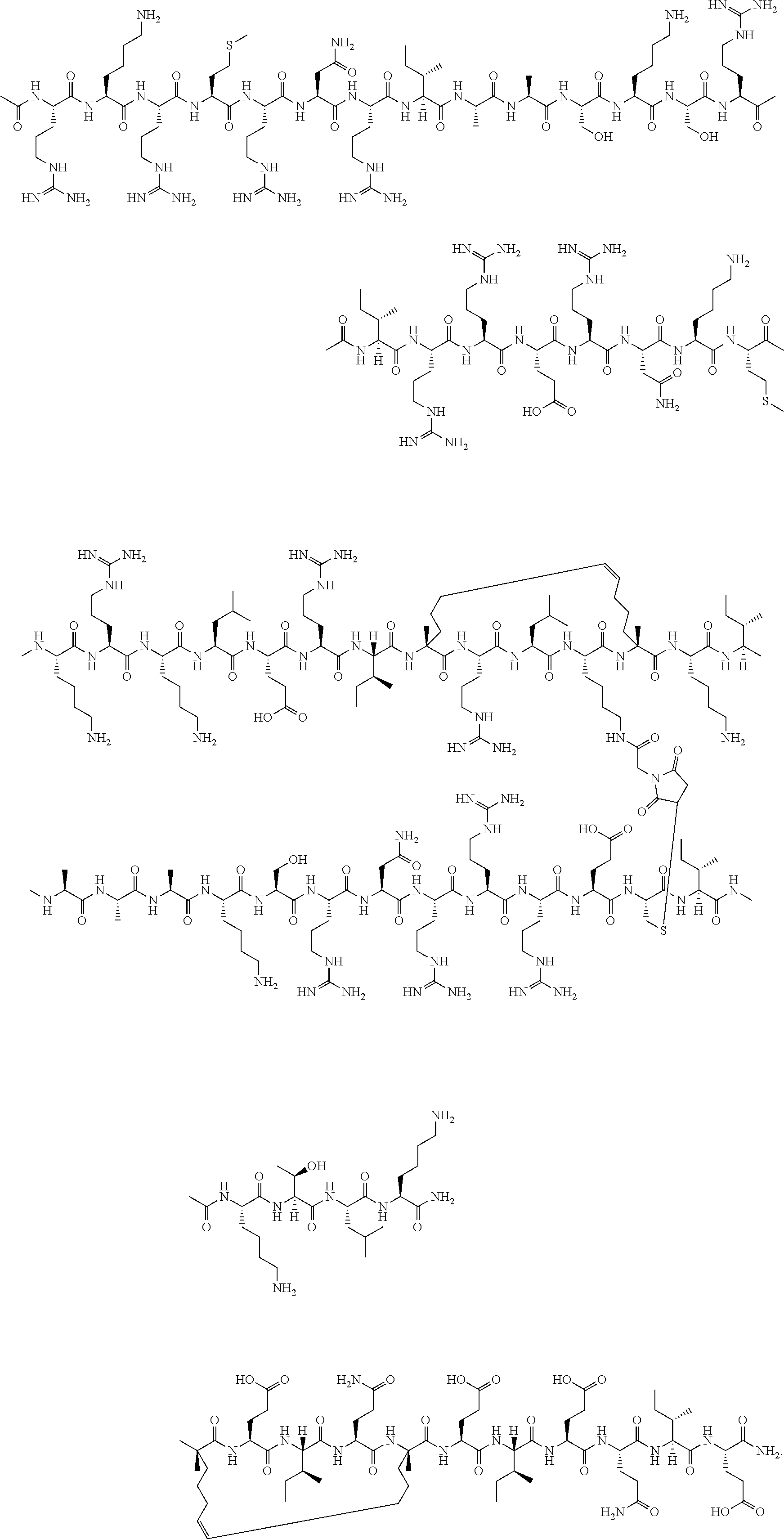

106. A method for modifying expression of a bZIP protein target gene in a cell, the method comprising providing to the cell the engineered DNA-binding dimer of any of claims 1-105.

107. The method of claim 106, wherein the bZIP protein target gene is c-Fos.

108. The method of claim 106, wherein the bZIP protein target gene is c-Jun.

109. The method of claim 106, wherein the bZIP protein target gene is XBP1.

110. The method of claim 106, wherein the bZIP protein target gene is ATF4.

111. The method of claim 106, wherein the bZIP protein target gene is ATF6.

112. The method of claim 106, wherein the bZIP protein target gene is C/EBPβ.

113. A method for introducing the engineered DNA-binding dimer of any of claims 1-105 into a cell, the method comprising culturing the cell with the engineered DNA-binding dimer.

114. A method for reducing an amount of a bZIP protein bound to DNA in a cell, the method comprising providing the engineered DNA-binding dimer of any of claims 1-105 to the cell.

115. The method of claim 114, wherein the bZIP protein is c-Fos.

116. The method of claim 114, wherein the bZIP protein is c-Jun.

117. The method of claim 114, wherein the bZIP protein is XBP1.

118. The method of claim 114, wherein the bZIP protein is ATF4.

119. The method of claim 114, wherein the bZIP protein is ATF6.

120. The method of claim 114, wherein the bZIP protein is C/EBPβ.

121. A method for treating a subject for a condition, the method comprising administering to the subject an effective amount of the engineered DNA-binding dimer of any of claims 1-105, wherein the condition is affected by expression of a gene under the control of a bZIP transcription factor.

122. The method of claim 121, wherein the condition is fibrosis.

123. The method of claim 122, wherein the fibrosis is liver fibrosis, renal fibrosis, cardiac fibrosis, pulmonary fibrosis, idiopathic pulmonary fibrosis (IPF), scleroderma, psoriasis, or myelofibrosis.

124. The method of claim 121, wherein the condition is cancer.

125. The method of claim 124, wherein the cancer is leukemia, lymphoma, myeloma, triple negative breast cancer, prostate cancer, pancreatic neuroendocrine tumors, pancreatic ductal adenocarcinoma, ovarian cancer, lung adenocarcinoma, liver cancer, glioblastoma, renal cell carcinoma.

126. The method of claim 124, wherein the cancer is breast cancer.

127. The method of claim 126, wherein the breast cancer is triple negative breast cancer.

128. The method of any of claims 124-127, further comprising administering to the subject an additional cancer therapy.

129. The method of claim 128, wherein the additional cancer therapy is chemotherapy, radiotherapy, immunotherapy, or a proteasome inhibitor.

130. The method of any of claims 124-129, wherein the subject was previously treated with a cancer therapy.

131. The method of claim 130, wherein the subject was determined to be resistant to the cancer therapy.

132. The method of claim 130 or 131, wherein the cancer therapy comprises chemotherapy, radiotherapy, immunotherapy, or a proteasome inhibitor.

133. The method of claim 121, wherein the condition is diabetes.

134. The method of claim 133, wherein the condition is type 1 diabetes.

135. The method of claim 133, wherein the condition is type 2 diabetes.

136. An engineered peptide having the sequence:
(SEQ ID NO: 59)
Ac-IRRERNKMAAAKSRNRRRECTDTLEDETDQLE-NH₂,
(SEQ ID NO: 60)
Ac-IRRERNKMAAAKSRNRRRECLDELQAEIEQLE-NH₂,
(SEQ ID NO: 61)
Ac-IRRERNKMAAAKSRNRRRECIDELQAEIEQLE-NH₂,
(SEQ ID NO: 62)
Ac-IRRERNKMAAAKSRNRRRECIDEIQAEIEQIE-NH₂,
(SEQ ID NO: 63)
Ac-IRR#RNK#AAAKSRNRRRECLDELQAEIEQLE-NH₂,
(SEQ ID NO: 64)
Ac-IRRERNKMAAAKSRNRRRECL#ELQ#EIEQLE-NH₂,
(SEQ ID NO: 65)
Ac-IRR#RNK#AAAKSRNRRRECIDELQAEIEQLE-NH₂,
(SEQ ID NO: 66)
Ac-IRRERNKMAAAKSRNRRRECI#ELQ#EIEQLE-NH₂,
(SEQ ID NO: 67)
Ac-IRR#RNK#AAAKSRNRRRECIDEIQAEIEQIE-NH₂,
(SEQ ID NO: 68)
Ac-IRRERNKMAAAKSRNRRRECI#EIQ#EIEQIE-NH₂,
(SEQ ID NO: 69)
Ac-EEKRRIRRERNKMAAAKSRNRRRECLDELQAEIEQLE-NH₂,
(SEQ ID NO: 70)
Ac-EEKRRIRR#RNK#AAAKSRNRRRECLDELQAEIEQLE-NH₂,
or
(SEQ ID NO: 71)
Ac-EEKRRIRRERNKMAAAKSRNRRRECL#ELQ#EIEQLE-NH₂,
wherein
Ac is acetyl; and
# is (S)-2-(4′-pentenyl)alanine.

137. The engineered peptide of claim 136, wherein the engineered peptide has the sequence Ac-IRRERNKMAAAKSRNRRRECI#EIQ#EIEQIE-NH₂ (SEQ ID NO:68).

138. An engineered peptide having the sequence:
(SEQ ID NO: 72)
Ac-RKRMRNRIAASKSRKRKLERIAKₘLEEKVKTLK-NH₂,
(SEQ ID NO: 73)
Ac-RKRMRNRIAASKSRKRKLERIARLKₘEKVKTLK-NH₂,
(SEQ ID NO: 74)
Ac-RKRMRNRIAASKSRKRKLERAAELKₘEKVATLK-NH₂,
(SEQ ID NO: 75)
Ac-RKRMRNRIAASKSRKRKLERIARLKₘEKIKTLK-NH₂,
(SEQ ID NO: 76)
Ac-RKRMRNRIAASKSRKRKLERIARIKₘEKIKTIK-NH₂,
(SEQ ID NO: 77)
Ac-RKR#RNR#AASKSRKRKLERIARLKₘEKVKTLK-NH₂,
(SEQ ID NO: 78)
Ac-RKRMRNRIAASKSRKRKLERI#RLKₘ#KVKTLK-NH₂,
(SEQ ID NO: 79)
Ac-RKR#RNR#AASKSRKRKLERIARLKₘEKIKTLK-NH₂,
(SEQ ID NO: 80)
Ac-RKRMRNRIAASKSRKRKLERI#RLKₘ#KIKTLK-NH₂,
(SEQ ID NO: 81)
Ac-RIKAERKRMRNRIAASKSRKRKLERIARLKₘEKVKTLK-NH₂,
(SEQ ID NO: 82)
Ac-RIKAERKR#RNR#AASKSRKRKLERIARLKₘEKVKTLK-NH₂,
or
(SEQ ID NO: 83)
Ac-RIKAERKRMRNRIAASKSRKRKLERI#RLKₘ#KVKTLK-NH₂,
wherein
Ac is acetyl;
# is (S)-2-(4′-pentenyl)alanine; and
Kₘ is Lys(Mmt) or a Lys residue linked to a maleimide linker.

139. The engineered peptide of claim 138, wherein the engineered peptide has the sequence Ac-RKRMRNRIAASKSRKRKLERI#RLKₘ#KIKTLK-NH₂ (SEQ ID NO:80).

140. A composition comprising (i) the engineered peptide of claim 136 or 137 and (ii) the engineered peptide of claim 138 or 139.

141. A method for generating an engineered DNA-binding dimer, the method comprising subjecting the composition of claim 140 to conditions sufficient to form a side-by-side interpeptide linkage between the engineered peptide of claim 136 or 137 and the engineered peptide of claim 138 or 139.

142. The method of claim 141, wherein the side-by-side interpeptide linkage comprises a maleimide-thiol adduct.

143. The method of claim 142, wherein the side-by-side interpeptide linkage is

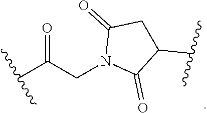 [SMILES: *C(=O)CN1C(=O)CC(*)C1=O]

144. The method of any of claims 141-143, wherein the engineered peptide of claim 136 or 137 and the engineered peptide of claim 138 or 139 each comprise two (S)-2-(4′-pentenyl)alanine residues, wherein the composition is further subjected to conditions sufficient to generate a intrapeptide stabilizing linkage between the two (S)-2-(4′-pentenyl)alanine residues.

145. An engineered peptide having the sequence:
(SEQ ID NO: 84)
Ac-RRKLKNRVAAQTARDRKKACMSELEQQVVDLE-NH₂,
(SEQ ID NO: 85)
Ac-RRKLKNRVAAQTARDRKKACLSELEQQVVDLE-NH₂,
(SEQ ID NO: 86)
Ac-RRKLKNRVAAQTARDRKKACL#ELE#QVVDLE-NH₂,
or
(SEQ ID NO: 87)
Ac-RRK#KNR#AAQTARDRKKACLSELEQQVVDLE-NH₂,
wherein
Ac is acetyl; and
# is (S)-2-(4′-pentenyl)alanine.

146. The engineered peptide of claim 145, wherein the engineered peptide has the sequence Ac-RRK#KNR#AAQTARDRKKACLSELEQQVVDLE-NH₂ (SEQ ID NO:87).

147. An engineered peptide having the sequence:
(SEQ ID NO: 88)
Ac-RRKLKNRVAAQTARDRKKARMSELKₘQQVVDLE-NH₂,
(SEQ ID NO: 89)
Ac-RRKLKNRVAAQTARDRKKARLSELKₘQQVVDLE-NH₂,
(SEQ ID NO: 90)
Ac-RRKLKNRVAAQTARDRKKARL#ELKₘ#QVVDLE-NH₂,
or
(SEQ ID NO: 91)
Ac-RRK#KNR#AAQTARDRKKARLSELKₘQQVVDLE-NH₂,
wherein
Ac is acetyl;
# is (S)-2-(4′-pentenyl)alanine; and
Kₘ is Lys(Mmt) or a Lys residue linked to a maleimide linker.

148. The engineered peptide of claim 147, wherein the engineered peptide has the sequence Ac-RRK#KNR#AAQTARDRKKARLSELKₘQQVVDLE-NH₂ (SEQ ID NO:91).

149. A composition comprising (i) the engineered peptide of claim 145 or 146 and (ii) the engineered peptide of claim 147 or 148.

150. A method for generating an engineered DNA-binding dimer, the method comprising subjecting the composition of claim 149 to conditions sufficient to form a side-by-side interpeptide linkage between the engineered peptide of claim 145 or 146 and the engineered peptide of claim 147 or 148.

151. The method of claim 150, wherein the side-by-side interpeptide linkage comprises a maleimide-thiol adduct.

152. The method of claim 151, wherein the side-by-side interpeptide linkage is

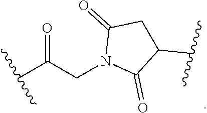 [SMILES: *C(=O)CN1C(=O)CC(*)C1=O]

153. The method of any of claims 150-152, wherein the engineered peptide of claim 145 or 146 and the engineered peptide of claim 147 or 148 each comprise two (S)-2-(4′-pentenyl)alanine residues, wherein the composition is further subjected to conditions sufficient to generate a intrapeptide stabilizing linkage between the two (S)-2-(4′-pentenyl)alanine residues.

154. An engineered peptide having the sequence:
(SEQ ID NO: 92)
Ac-KKMEQNKTAATRYRQKKRACQEALTGELKELE-NH₂,
(SEQ ID NO: 93)
Ac-KKMEQNKTAATRYRQKKRACLEALKAELKELR-NH₂,
(SEQ ID NO: 94)
Ac-KKMEQNKTAATRYRQKKRACL#ALK#ELKELR-NH₂,
wherein
Ac is acetyl; and
# is (S)-2-(4′-pentenyl)alanine.

155. The engineered peptide of claim 154, wherein the engineered peptide has the sequence Ac-KKMEQNKTAATRYRQKKRACQEALTGELKELE-NH₂ (SEQ ID NO:92).

156. An engineered peptide having the sequence:
(SEQ ID NO: 95)
Ac-KKMEQNKTAATRYRQKKRAEQEALKmGELKELE-NH₂,
(SEQ ID NO: 96)
Ac-KKMEQNKTAATRYRQKKRAELEALKmAELKELR-NH₂,
or
(SEQ ID NO: 97)
Ac-KKMEQNKTAATRYRQKKRAEL#ALKm#ELKELR-NH₂,
wherein
Ac is acetyl;
# is (S)-2-(4′-pentenyl)alanine; and
Kₘ is Lys(Mmt) or a Lys residue linked to a maleimide linker.

157. The engineered peptide of claim 156, wherein the engineered peptide has the sequence Ac-KKMEQNKTAATRYRQKKRAEL#ALKₘ#ELKELR-NH₂ (SEQ ID NO:97).

158. A composition comprising (i) the engineered peptide of claim 154 or 155 and (ii) the engineered peptide of claim 156 or 157.

159. A method for generating an engineered DNA-binding dimer, the method comprising subjecting the composition of claim 158 to conditions sufficient to form a side-by-side interpeptide linkage between the engineered peptide of claim 154 or 155 and the engineered peptide of claim 156 or 157.

160. The method of claim 159, wherein the side-by-side interpeptide linkage comprises a maleimide-thiol adduct.

161. The method of claim 160, wherein the side-by-side interpeptide linkage is

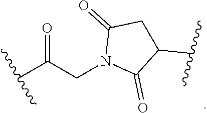 [SMILES: *C(=O)CN1C(=O)CC(*)C1=O]

162. The method of any of claims 159-161, wherein the engineered peptide of claim 154 or 155 and the engineered peptide of claim 156 or 157 each comprise two (S)-2-(4′-pentenyl)alanine residues, wherein the composition is further subjected to conditions sufficient to generate a intrapeptide stabilizing linkage between the two (S)-2-(4′-pentenyl)alanine residues.

163. An engineered peptide having the sequence:
(SEQ ID NO: 98)
Ac-IRRERNNIAVRKSRDKAKMCLLELQHKVLELR-NH₂,
or
(SEQ ID NO: 99)
Ac-IRRERNNIAVRKSRDKAKMCL#ELQ#KVLELR-NH₂,
wherein
Ac is acetyl; and
# is (S)-2-(4′-pentenyl)alanine.

164. The engineered peptide of claim 163, wherein the engineered peptide has the sequence Ac-IRRERNNIAVRKSRDKAKMCL#ELQ#KVLELR-NH₂ (SEQ ID NO:99).

165. A composition comprising (i) the engineered peptide of claim 163 or 164 and (ii) the engineered peptide of claim 156 or 157.

166. A method for generating an engineered DNA-binding dimer, the method comprising subjecting the composition of claim 165 to conditions sufficient to form a side-by-side interpeptide linkage between the engineered peptide of claim 163 or 164 and the engineered peptide of claim 156 or 157.

167. The method of claim 166, wherein the side-by-side interpeptide linkage comprises a maleimide-thiol adduct.

168. The method of claim 167, wherein the side-by-side interpeptide linkage is

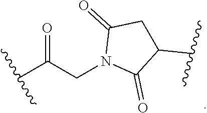 [SMILES: *C(=O)CN1C(=O)CC(*)C1=O]

169. The method of any of claims 166-168, wherein the engineered peptide of claim 163 or 164 and the engineered peptide of claim 156 or 157 each comprise two (S)-2-(4′-pentenyl)alanine residues, wherein the composition is further subjected to conditions sufficient to generate a intrapeptide stabilizing linkage between the two (S)-2-(4′-pentenyl)alanine residues.

170. A method of reducing expression of a HIF protein target gene in a cell, the method comprising providing to the cell an engineered DNA-binding dimer comprising:
(a) a first engineered peptide comprising (i) a modified basic domain sequence of XBP1 and (ii) a modified leucine zipper domain sequence of XBP1; and,
(b) a second engineered peptide linked to the first engineered peptide via a side-by-side interpeptide linkage, the second engineered peptide comprising (i) a modified basic domain sequence of XBP1 and (ii) a modified leucine zipper domain sequence of XBP1.

171. The method of claim 170, wherein the engineered DNA-binding dimer is provided in an amount effective to reduce expression of GLUT1 in the cell.

172. The method of claim 170, wherein the engineered DNA-binding dimer is provided in an amount effective to reduce expression of VEGFA in the cell.

173. The method of claim 170, wherein the engineered DNA-binding dimer is provided in an amount effective to reduce expression of PGK1 in the cell.

174. The method of any of claims 170-173, wherein the HIF protein is HIF1α.

175. The method of any of claims 170-173, wherein the HIF protein is HIF2α.

176. The method of any of claims 170-173, wherein the engineered DNA-binding dimer has formula.

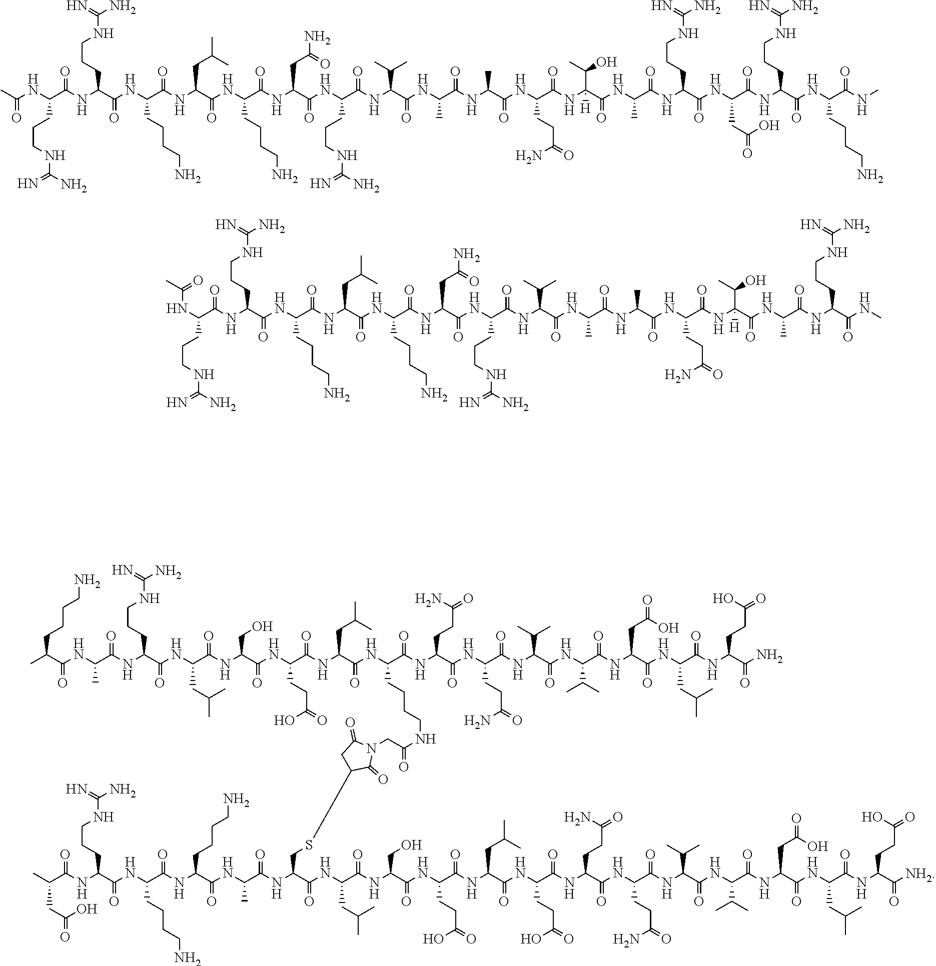

177. The method of any of claims 170-173, wherein the engineered DNA-binding dimer has formula.

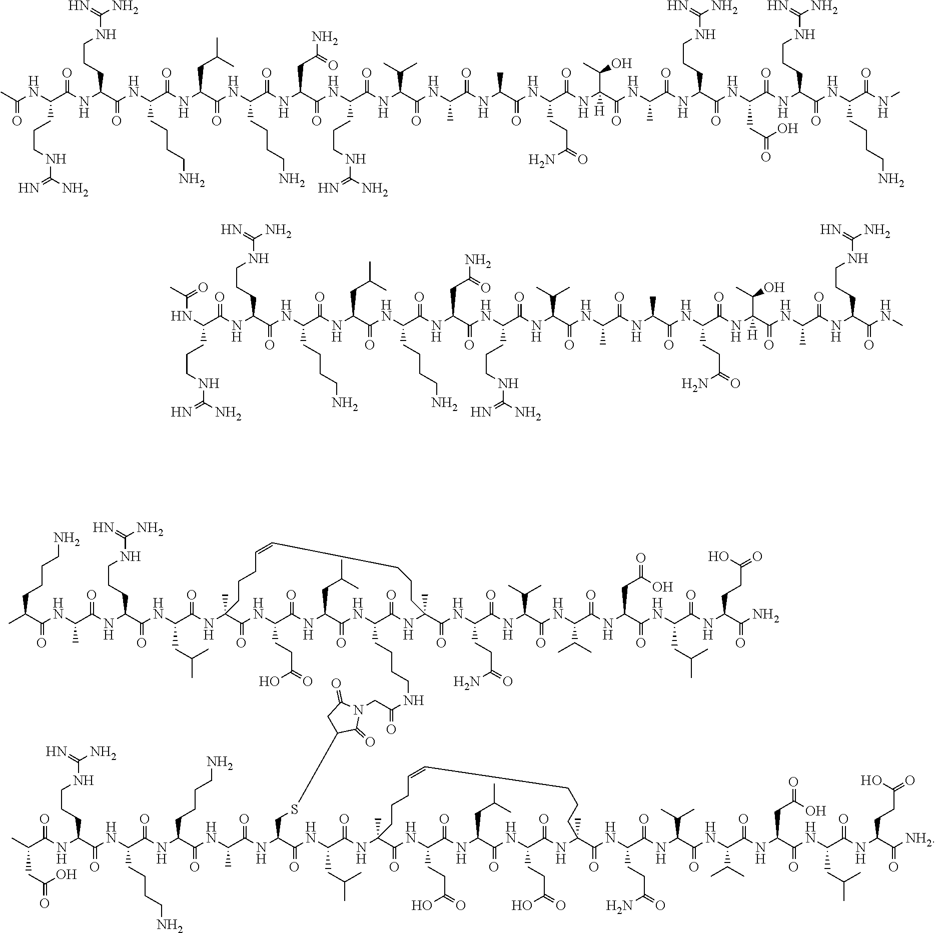

178. The method of any of claims 170-173, wherein the engineered DNA-binding dimer has formula.

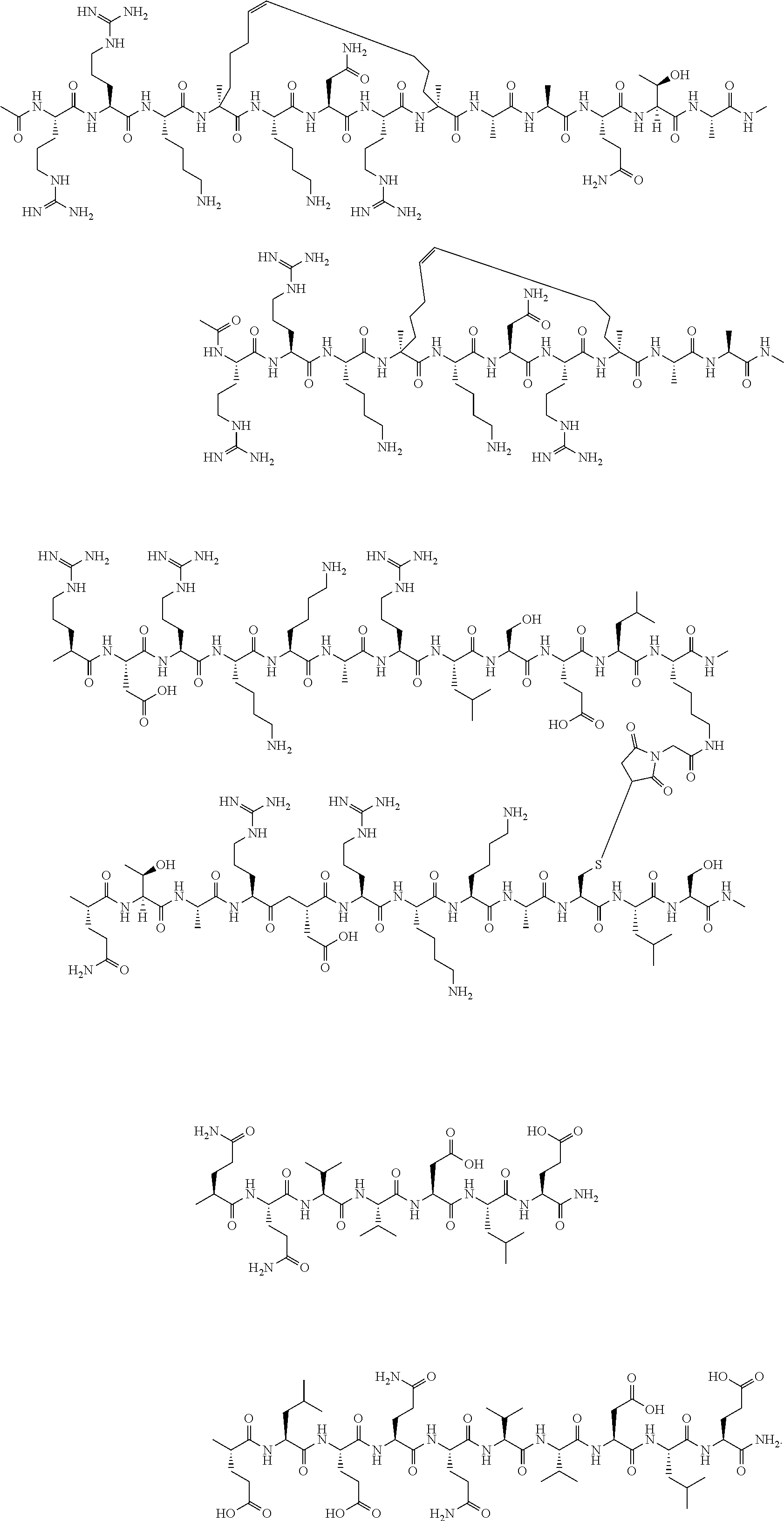

179. The method of any of claims 170-178, wherein the cell is a cancer cell.

180. The method of any of claims 170-178, wherein the cell is a breast cancer cell.

181. The method of any of claims 170-178, wherein the breast cancer cell is a triple negative breast cancer cell.


SEQUENCE LISTING (166 sequences)

SEQ ID NO: 1 - protein, 20 aa, synthetic construct
IRRERNKMAAAKCRNRRREL

SEQ ID NO: 2 - protein, 12 aa, synthetic construct
TDTLQAETDQLE

SEQ ID NO: 3 - protein, 380 aa, synthetic construct
MMFSGFNADYEASSSRCSSASPAGDSLSYYHSPADSFSSMGSPVNAQDFCTDLAVSSANFIPTVTAISTSPDLQWLVQPALVSSVAPSQTRAPHPFGVPAPSAGAYSRAGVVKTMTGGRAQSIGRRGKVEQLSPEEEEKRRIRRERNKMAAAKCRNRRRELTDTLQAETDQLEDEKSALQTEIANLLKEKEKLEFILAAHRPACKIPDDLGFPEEMSVASLDLTGGLPEVATPESEEAFTLPLLNDPEPKPSVEPVKSISSMELKTEPFDDFLFPASSRPSGSETARSVPDMDLSGSFYAADWEPLHSGSLGMGPMATELEPLCTPVVTCTPSCTAYTSSFVFTYPEADSFPSCAAAHRKGSSSNEPSSDSLSSPTLLAL

SEQ ID NO: 4 - protein, 20 aa, synthetic construct
RKRMRNRIAASKCRKRKLER

SEQ ID NO: 5 - protein, 12 aa, synthetic construct
IARLEEKVKTLK

SEQ ID NO: 6 - protein, 331 aa, synthetic construct
MTAKMETTFYDDALNASFLPSESGPYGYSNPKILKQSMTLNLADPVGSLKPHLRAKNSDLLTSPDVGLLKLASPELERLIIQSSNGHITTTPTPTQFLCPKNVTDEQEGFAEGFVRALAELHSQNTLPSVTSAAQPVNGAGMVAPAVASVAGGSGSGGFSASLHSEPPVYANLSNFNPGALSSGGGAPSYGAAGLAFPAQPQQQQQPPHHLPQQMPVQHPRLQALKEEPQTVPEMPGETPPLSPIDMESQERIKAERKRMRNRIAASKCRKRKLERIARLEEKVKTLKAQNSELASTANMLREQVAQLKQKVMNHVNSGCQLMLTQQLQTF

SEQ ID NO: 7 - protein, 20 aa, synthetic construct
RRKLKNRVAAQTARDRKKAR

SEQ ID NO: 8 - protein, 12 aa, synthetic construct
MSELEQQVVDLE

SEQ ID NO: 9 - protein, 261 aa, synthetic construct
MVVVAAAPNPADGTPKVLLLSGQPASAAGAPAGQALPLMVPAQRGASPEAASGGLPQARKRQRLTHLSPEEKALRRKLKNRVAAQTARDRKKARMSELEQQVVDLEEENQKLLLENQLLREKTHGLVVENQELRQRLGMDALVAEEEAEAKGNEVRPVAGSAESAALRLRAPLQQVQAQLSPLQNISPWILAVLTLQIQSLISCWAFWTTWTQSCSSNALPQSLPAWRSSQRSTQKDPVPYQPPFLCQWGRHQPSWKPLMN

SEQ ID NO: 10 - protein, 20 aa, synthetic construct
KKMEQNKTAATRYRQKKRAE

SEQ ID NO: 11 - protein, 12 aa, synthetic construct
QEALTGECKELE

SEQ ID NO: 12 - protein, 351 aa, synthetic construct
MTEMSFLSSEVLVGDLMSPFDQSGLGAEESLGLLDDYLEVAKHFKPHGFSSDKAKAGSSEWLAVDGLVSPSNNSKEDAFSGTDWMLEKMDLKEFDLDALLGIDDLETMPDDLLTTLDDTCDLFAPLVQETNKQPPQTVNPIGHLPESLTKPDQVAPFTFLQPLPLSPGVLSSTPDHSFSLELGSEVDITEGDRKPDYTAYVAMIPQCIKEEDTPSDNDSGICMSPESYLGSPQHSPSTRGSPNRSLPSPGVLCGSARPKPYDPPGEKMVAAKVKGEKLDKKLKKMEQNKTAATRYRQKKRAEQEALTGECKELEKKNEALKERADSLAKEIQYLKDLIEEVRKARGKKRVP

SEQ ID NO: 13 - protein, 20 aa, synthetic construct
IRRERNNIAVRKSRDKAKMR

SEQ ID NO: 14 - protein, 12 aa, synthetic construct
NLETQHKVLELT

SEQ ID NO: 15 - protein, 345 aa, synthetic construct
MQRLVAWDPACLPLPPPPPAFKSMEVANFYYEADCLAAAYGGKAAPAAPPAARPGPRPPAGELGSIGDHERAIDFSPYLEPLGAPQAPAPATATDTFEAAPPAPAPAPASSGQHHDFLSDLFSDDYGGKNCKKPAEYGYVSLGRLGAAKGALHPGCFAPLHPPPPPPPPPAELKAEPGFEPADCKRKEEAGAPGGGAGMAAGFPYALRAYLGYQAVPSGSSGSLSTSSSSSPPGTPSPADAKAPPTACYAGAAPAPSQVKSKAKKTVDKHSDEYKIRRERNNIAVRKSRDKAKMRNLETQHKVLELTAENERLQKKVEQLSRELSTLRNLFKQLPEPLLASSGHC

SEQ ID NO: 16 - protein, 20 aa, synthetic construct
IRRERNKMAAAKSRNRRREC

SEQ ID NO: 17 - protein, 20 aa, synthetic construct
  CROSSLNK 4..8: note = intrapeptide stabilizing linkage
  MOD_RES 4: note = (S)-2-(4'-pentenyl)alanine
  MOD_RES 8: note = (S)-2-(4'-pentenyl)alanine
IRRXRNKXAAAKSRNRRREC

SEQ ID NO: 18 - protein, 25 aa, synthetic construct
EEKRRIRRERNKMAAAKSRNRRREC

SEQ ID NO: 19 - protein, 25 aa, synthetic construct
  CROSSLNK 9..13: note = intrapeptide stabilizing linkage
  MOD_RES 9: note = (S)-2-(4'-pentenyl)alanine
  MOD_RES 13: note = (S)-2-(4'-pentenyl)alanine
EEKRRIRRXRNKXAAAKSRNRRREC

SEQ ID NO: 20 - protein, 12 aa, synthetic construct
TDTLEDETDQLE

SEQ ID NO: 21 - protein, 12 aa, synthetic construct
LDELQAEIEQLE

SEQ ID NO: 22 - protein, 12 aa, synthetic construct
IDELQAEIEQLE

SEQ ID NO: 23 - protein, 12 aa, synthetic construct
IDEIQAEIEQIE

SEQ ID NO: 24 - protein, 12 aa, synthetic construct
  CROSSLNK 2..6: note = Intrapeptide stabilizing linkage
  MOD_RES 2: note = (S)-2-(4'-pentenyl)alanine
  MOD_RES 6: note = (S)-2-(4'-pentenyl)alanine
LXELQXEIEQLE

SEQ ID NO: 25 - protein, 12 aa, synthetic construct
  CROSSLNK 2..6: note = Intrapeptide stabilizing linkage
  MOD_RES 2: note = (S)-2-(4'-pentenyl)alanine
  MOD_RES 6: note = (S)-2-(4'-pentenyl)alanine
IXELQXEIEQLE

SEQ ID NO: 26 - protein, 12 aa, synthetic construct
  CROSSLNK 2..6: note = Intrapeptide stabilizing linkage
  MOD_RES 2: note = (S)-2-(4'-pentenyl)alanine
  MOD_RES 6: note = (S)-2-(4'-pentenyl)alanine
IXEIQXEIEQIE

SEQ ID NO: 27 - protein, 20 aa, synthetic construct
RKRMRNRIAASKSRKRKLER

SEQ ID NO: 28 - protein, 20 aa, synthetic construct
  CROSSLNK 4..8: note = Intrapeptide stabilizing linkage
  MOD_RES 4: note = (S)-2-(4'-pentenyl)alanine
  MOD_RES 8: note = (S)-2-(4'-pentenyl)alanine
RKRXRNRXAASKSRKRKLER

SEQ ID NO: 29 - protein, 25 aa, synthetic construct
RIKAERKRMRNRIAASKSRKRKLER

SEQ ID NO: 30 - protein, 25 aa, synthetic construct
  CROSSLNK 9..13: note = Intrapeptide stabilizing linkage
  MOD_RES 9: note = (S)-2-(4'-pentenyl)alanine
  MOD_RES 13: note = (S)-2-(4'-pentenyl)alanine
RIKAERKRXRNRXAASKSRKRKLER

SEQ ID NO: 31 - protein, 12 aa, synthetic construct
  MOD_RES 3: note = N-(4methoxypheyl)diphenylmethyl-lysine
IAXLEEKVKTLK

SEQ ID NO: 32 - protein, 12 aa, synthetic construct
  MOD_RES 5: note = N-(4methoxypheyl)diphenylmethyl-lysine
IARLXEKVKTLK

SEQ ID NO: 33 - protein, 12 aa, synthetic construct
  MOD_RES 5: note = N-(4methoxypheyl)diphenylmethyl-lysine
AAELXEKVATLK

SEQ ID NO: 34 - protein, 12 aa, synthetic construct
  MOD_RES 5: note = N-(4methoxypheyl)diphenylmethyl-lysine
IARLXEKIKTLK

SEQ ID NO: 35 - protein, 12 aa, synthetic construct
  MOD_RES 5: note = N-(4methoxypheyl)diphenylmethyl-lysine
IARIXEKIKTIK

SEQ ID NO: 36 - protein, 12 aa, synthetic construct
  MOD_RES 5: note = N-(4methoxypheyl)diphenylmethyl-lysine
  MOD_RES 2: note = (S)-2-(4'-pentenyl)alanine
  MOD_RES 6: note = (S)-2-(4'-pentenyl)alanine
  CROSSLNK 2..6: note = Intrapeptide stabilizing linkage
IXRLXXKVKTLK

SEQ ID NO: 37 - protein, 12 aa, synthetic construct
  CROSSLNK 2..6: note = Intrapeptide stabilizing linkage
  MOD_RES 2: note = (S)-2-(4'-pentenyl)alanine
  MOD_RES 6: note = (S)-2-(4'-pentenyl)alanine
  MOD_RES 5: note = N-(4methoxypheyl)diphenylmethyl-lysine
IXRLXXKIKTLK

SEQ ID NO: 38 - protein, 20 aa, synthetic construct
RRKLKNRVAAQTARDRKKAC

SEQ ID NO: 39 - protein, 20 aa, synthetic construct
  MOD_RES 4: note = (S)-2-(4'-pentenyl)alanine
  MOD_RES 8: note = (S)-2-(4'-pentenyl)alanine
  CROSSLNK 4..8: note = Intrapeptide stabilizing linkage
RRKXKNRXAAQTARDRKKAC

SEQ ID NO: 40 - protein, 12 aa, synthetic construct
MSELEQQVVDLE

SEQ ID NO: 41 - protein, 12 aa, synthetic construct
LSELEQQVVDLE

SEQ ID NO: 42 - protein, 12 aa, synthetic construct
  CROSSLNK 2..6: note = Intrapeptide stabilizing linkage
  MOD_RES 2: note = (S)-2-(4'-pentenyl)alanine
  MOD_RES 6: note = (S)-2-(4'-pentenyl)alanine
LXELEXQVVDLE

SEQ ID NO: 43 - protein, 20 aa, synthetic construct
RRKLKNRVAAQTARDRKKAR

SEQ ID NO: 44 - protein, 20 aa, synthetic construct
  CROSSLNK 4..8: note = Intrapeptide stabilizing linkage
  MOD_RES 4: note = (S)-2-(4'-pentenyl)alanine
  MOD_RES 8: note = (S)-2-(4'-pentenyl)alanine
RRKXKNRXAAQTARDRKKAR

SEQ ID NO: 45 - protein, 12 aa, synthetic construct
  MOD_RES 5: note = N-(4methoxypheyl)diphenylmethyl-lysine
MSELXQQVVDLE

SEQ ID NO: 46 - protein, 12 aa, synthetic construct
  MOD_RES 5: note = N-(4methoxypheyl)diphenylmethyl-lysine
LSELXQQVVDLE

SEQ ID NO: 47 - protein, 12 aa, synthetic construct
  MOD_RES 2: note = (S)-2-(4'-pentenyl)alanine
  MOD_RES 6: note = (S)-2-(4'-pentenyl)alanine
  MOD_RES 5: note = N-(4methoxypheyl)diphenylmethyl-lysine
  CROSSLNK 2..6: note = Intrapeptide stabilizing linkage
LXELXXQVVDLE

SEQ ID NO: 48 - protein, 20 aa, synthetic construct
KKMEQNKTAATRYRQKKRAC

SEQ ID NO: 49 - protein, 12 aa, synthetic construct
QEALTGELKELE

SEQ ID NO: 50 - protein, 12 aa, synthetic construct
LEALKAELKELR

SEQ ID NO: 51 - protein, 12 aa, synthetic construct
  CROSSLNK 2..6: note = Intrapeptide stabilizing linkage
  MOD_RES 2: note = (S)-2-(4'-pentenyl)alanine
  MOD_RES 6: note = (S)-2-(4'-pentenyl)alanine
LXALKXELKELR

SEQ ID NO: 52 - protein, 20 aa, synthetic construct
IRRERNNIAVRKSRDKAKMC

SEQ ID NO: 53 - protein, 12 aa, synthetic construct
LLELQHKVLELR

SEQ ID NO: 54 - protein, 12 aa, synthetic construct
  MOD_RES 2: note = (S)-2-(4'-pentenyl)alanine
  MOD_RES 6: note = (S)-2-(4'-pentenyl)alanine
  CROSSLNK 2..6: note = Intrapeptide stabilizing linkage
LXELQXKVLELR

SEQ ID NO: 55 - protein, 20 aa, synthetic construct
KKMEQNKTAATRYRQKKRAE

SEQ ID NO: 56 - protein, 12 aa, synthetic construct
  MOD_RES 5: note = N-(4methoxypheyl)diphenylmethyl-lysine
QEALXGELKELE

SEQ ID NO: 57 - protein, 12 aa, synthetic construct
  MOD_RES 5: note = N-(4methoxypheyl)diphenylmethyl-lysine
LEALXAELKELR

SEQ ID NO: 58 - protein, 12 aa, synthetic construct
  CROSSLNK 2..6: note = Intrapeptide stabilizing linkage
  MOD_RES 2: note = (S)-2-(4'-pentenyl)alanine
  MOD_RES 6: note = (S)-2-(4'-pentenyl)alanine
  MOD_RES 5: note = N-(4methoxypheyl)diphenylmethyl-lysine
LXALXXELKELR

SEQ ID NO: 59 - protein, 32 aa, synthetic construct
IRRERNKMAAAKSRNRRRECTDTLEDETDQLE

SEQ ID NO: 60 - protein, 32 aa, synthetic construct
IRRERNKMAAAKSRNRRRECLDELQAEIEQLE

SEQ ID NO: 61 - protein, 32 aa, synthetic construct
IRRERNKMAAAKSRNRRRECIDELQAEIEQLE

SEQ ID NO: 62 - protein, 32 aa, synthetic construct
IRRERNKMAAAKSRNRRRECIDEIQAEIEQIE

SEQ ID NO: 63 - protein, 32 aa, synthetic construct
  CROSSLNK 4..8: note = Intrapeptide stabilizing linkage
  MOD_RES 4: note = (S)-2-(4'-pentenyl)alanine
  MOD_RES 8: note = (S)-2-(4'-pentenyl)alanine
IRRXRNKXAAAKSRNRRRECLDELQAEIEQLE

SEQ ID NO: 64 - protein, 32 aa, synthetic construct
  MOD_RES 22: note = (S)-2-(4'-pentenyl)alanine
  MOD_RES 26: note = (S)-2-(4'-pentenyl)alanine
  CROSSLNK 22..26: note = Intrapeptide stabilizing linkage
IRRERNKMAAAKSRNRRRECLXELQXEIEQLE

SEQ ID NO: 65 - protein, 32 aa, synthetic construct
  CROSSLNK 4..8: note = Intrapeptide stabilizing linkage
  MOD_RES 4: note = (S)-2-(4'-pentenyl)alanine
  MOD_RES 8: note = (S)-2-(4'-pentenyl)alanine
IRRXRNKXAAAKSRNRRRECIDELQAEIEQLE

SEQ ID NO: 66 - protein, 32 aa, synthetic construct
  MOD_RES 22: note = (S)-2-(4'-pentenyl)alanine
  MOD_RES 26: note = (S)-2-(4'-pentenyl)alanine
  CROSSLNK 22..26: note = Intrapeptide stabilizing linkage
IRRERNKMAAAKSRNRRRECIXELQXEIEQLE

SEQ ID NO: 67 - protein, 32 aa, synthetic construct
  CROSSLNK 4..8: note = Intrapeptide stabilizing linkage
  MOD_RES 4: note = (S)-2-(4'-pentenyl)alanine
  MOD_RES 8: note = (S)-2-(4'-pentenyl)alanine
IRRXRNKXAAAKSRNRRRECIDEIQAEIEQIE

SEQ ID NO: 68 - protein, 32 aa, synthetic construct
  MOD_RES 22: note = (S)-2-(4'-pentenyl)alanine
  MOD_RES 26: note = (S)-2-(4'-pentenyl)alanine
  CROSSLNK 22..26: note = Intrapeptide stabilizing linkage
IRRERNKMAAAKSRNRRRECIXEIQXEIEQIE

SEQ ID NO: 69 - protein, 37 aa, synthetic construct
EEKRRIRRERNKMAAAKSRNRRRECLDELQAEIEQLE

SEQ ID NO: 70 - protein, 37 aa, synthetic construct
  CROSSLNK 9..13: note = Intrapeptide stabilizing linkage
  MOD_RES 9: note = (S)-2-(4'-pentenyl)alanine
  MOD_RES 13: note = (S)-2-(4'-pentenyl)alanine
EEKRRIRRXRNKXAAAKSRNRRRECLDELQAEIEQLE

SEQ ID NO: 71 - protein, 37 aa, synthetic construct
  CROSSLNK 27..31: note = Intrapeptide stabilizing linkage
  MOD_RES 27: note = (S)-2-(4'-pentenyl)alanine
  MOD_RES 31: note = (S)-2-(4'-pentenyl)alanine
EEKRRIRRERNKMAAAKSRNRRRECLXELQXEIEQLE

SEQ ID NO: 72 - protein, 32 aa, synthetic construct
  MOD_RES 23: note = N-(4methoxypheyl)diphenylmethyl-lysine
RKRMRNRIAASKSRKRKLERIAXLEEKVKTLK

SEQ ID NO: 73 - protein, 32 aa, synthetic construct
  MOD_RES 25: note = N-(4methoxypheyl)diphenylmethyl-lysine
RKRMRNRIAASKSRKRKLERIARLXEKVKTLK

SEQ ID NO: 74 - protein, 32 aa, synthetic construct
  MOD_RES 25: note = N-(4methoxypheyl)diphenylmethyl-lysine
RKRMRNRIAASKSRKRKLERAAELXEKVATLK

SEQ ID NO: 75 - protein, 32 aa, synthetic construct
  MOD_RES 25: note = N-(4methoxypheyl)diphenylmethyl-lysine
RKRMRNRIAASKSRKRKLERIARLXEKIKTLK

SEQ ID NO: 76 - protein, 32 aa, synthetic construct
  MOD_RES 25: note = N-(4methoxypheyl)diphenylmethyl-lysine
RKRMRNRIAASKSRKRKLERIARIXEKIKTIK

SEQ ID NO: 77 - protein, 32 aa, synthetic construct
  MOD_RES 25: note = N-(4methoxypheyl)diphenylmethyl-lysine
  MOD_RES 4: note = (S)-2-(4'-pentenyl)alanine
  MOD_RES 8: note = (S)-2-(4'-pentenyl)alanine
  CROSSLNK 4..8: note = Intrapeptide stabilizing linkage
RKRXRNRXAASKSRKRKLERIARLXEKVKTLK

SEQ ID NO: 78 - protein, 32 aa, synthetic construct
  CROSSLNK 22..26: note = Intrapeptide stabilizing linkage
  MOD_RES 22: note = (S)-2-(4'-pentenyl)alanine
  MOD_RES 26: note = (S)-2-(4'-pentenyl)alanine
  MOD_RES 25: note = N-(4methoxypheyl)diphenylmethyl-lysine
RKRMRNRIAASKSRKRKLERIXRLXXKVKTLK

SEQ ID NO: 79 - protein, 32 aa, synthetic construct
  MOD_RES 25: note = N-(4methoxypheyl)diphenylmethyl-lysine
  MOD_RES 4: note = (S)-2-(4'-pentenyl)alanine
  MOD_RES 8: note = (S)-2-(4'-pentenyl)alanine
  CROSSLNK 4..8: note = Intrapeptide stabilizing linkage
RKRXRNRXAASKSRKRKLERIARLXEKIKTLK

SEQ ID NO: 80 - protein, 32 aa, synthetic construct
  MOD_RES 25: note = N-(4methoxypheyl)diphenylmethyl-lysine
  MOD_RES 22: note = (S)-2-(4'-pentenyl)alanine
  MOD_RES 26: note = (S)-2-(4'-pentenyl)alanine
  CROSSLNK 22..26: note = Intrapeptide stabilizing linkage
RKRMRNRIAASKSRKRKLERIXRLXXKIKTLK

SEQ ID NO: 81 - protein, 37 aa, synthetic construct
  MOD_RES 30: note = N-(4methoxypheyl)diphenylmethyl-lysine
RIKAERKRMRNRIAASKSRKRKLERIARLXEKVKTLK

SEQ ID NO: 82 - protein, 37 aa, synthetic construct
  MOD_RES 30: note = N-(4methoxypheyl)diphenylmethyl-lysine
  MOD_RES 9: note = (S)-2-(4'-pentenyl)alanine
  MOD_RES 13: note = (S)-2-(4'-pentenyl)alanine
  CROSSLNK 9..13: note = Intrapeptide stabilizing linkage
RIKAERKRXRNRXAASKSRKRKLERIARLXEKVKTLK

SEQ ID NO: 83 - protein, 37 aa, synthetic construct
  CROSSLNK 27..31: note = Intrapeptide stabilizing linkage
  MOD_RES 27: note = (S)-2-(4'-pentenyl)alanine
  MOD_RES 31: note = (S)-2-(4'-pentenyl)alanine
  MOD_RES 30: note = N-(4methoxypheyl)diphenylmethyl-lysine
RIKAERKRMRNRIAASKSRKRKLERIXRLXXKVKTLK

SEQ ID NO: 84 - protein, 32 aa, synthetic construct
RRKLKNRVAAQTARDRKKACMSELEQQVVDLE

SEQ ID NO: 85 - protein, 32 aa, synthetic construct
RRKLKNRVAAQTARDRKKACLSELEQQVVDLE

SEQ ID NO: 86 - protein, 32 aa, synthetic construct
  CROSSLNK 22..26: note = Intrapeptide stabilizing linkage
  MOD_RES 22: note = (S)-2-(4'-pentenyl)alanine
  MOD_RES 26: note = (S)-2-(4'-pentenyl)alanine
RRKLKNRVAAQTARDRKKACLXELEXQVVDLE

SEQ ID NO: 87 - protein, 32 aa, synthetic construct
  MOD_RES 4: note = (S)-2-(4'-pentenyl)alanine
  MOD_RES 8: note = (S)-2-(4'-pentenyl)alanine
  CROSSLNK 4..8: note = Intrapeptide stabilizing linkage
RRKXKNRXAAQTARDRKKACLSELEQQVVDLE

SEQ ID NO: 88 - protein, 32 aa, synthetic construct
  MOD_RES 25: note = N-(4methoxypheyl)diphenylmethyl-lysine
RRKLKNRVAAQTARDRKKARMSELXQQVVDLE

SEQ ID NO: 89 - protein, 32 aa, synthetic construct
  MOD_RES 25: note = N-(4methoxypheyl)diphenylmethyl-lysine
RRKLKNRVAAQTARDRKKARLSELXQQVVDLE

SEQ ID NO: 90 - protein, 32 aa, synthetic construct
  MOD_RES 25: note = N-(4methoxypheyl)diphenylmethyl-lysine
  MOD_RES 22: note = (S)-2-(4'-pentenyl)alanine
  MOD_RES 26: note = (S)-2-(4'-pentenyl)alanine
  CROSSLNK 22..26: note = Intrapeptide stabilizing linkage
RRKLKNRVAAQTARDRKKARLXELXXQVVDLE

SEQ ID NO: 91 - protein, 32 aa, synthetic construct
  CROSSLNK 4..8: note = Intrapeptide stabilizing linkage
  MOD_RES 4: note = (S)-2-(4'-pentenyl)alanine
  MOD_RES 8: note = (S)-2-(4'-pentenyl)alanine
  MOD_RES 25: note = N-(4methoxypheyl)diphenylmethyl-lysine
RRKXKNRXAAQTARDRKKARLSELXQQVVDLE

SEQ ID NO: 92 - protein, 32 aa, synthetic construct
KKMEQNKTAATRYRQKKRACQEALTGELKELE

SEQ ID NO: 93 - protein, 32 aa, synthetic construct
KKMEQNKTAATRYRQKKRACLEALKAELKELR

SEQ ID NO: 94 - protein, 32 aa, synthetic construct
  MOD_RES 22: note = (S)-2-(4'-pentenyl)alanine
  MOD_RES 26: note = (S)-2-(4'-pentenyl)alanine
  CROSSLNK 22..26: note = Intrapeptide stabilizing linkage
KKMEQNKTAATRYRQKKRACLXALKXELKELR

SEQ ID NO: 95 - protein, 32 aa, synthetic construct
  MOD_RES 25: note = N-(4methoxypheyl)diphenylmethyl-lysine
KKMEQNKTAATRYRQKKRAEQEALXGELKELE

SEQ ID NO: 96 - protein, 32 aa, synthetic construct
  MOD_RES 25: note = N-(4methoxypheyl)diphenylmethyl-lysine
KKMEQNKTAATRYRQKKRAELEALXAELKELR

SEQ ID NO: 97 - protein, 32 aa, synthetic construct
  MOD_RES 25: note = N-(4methoxypheyl)diphenylmethyl-lysine
  MOD_RES 22: note = (S)-2-(4'-pentenyl)alanine
  MOD_RES 26: note = (S)-2-(4'-pentenyl)alanine
  CROSSLNK 22..26: note = Intrapeptide stabilizing linkage
KKMEQNKTAATRYRQKKRAELXALXXELKELR

SEQ ID NO: 98 - protein, 32 aa, synthetic construct
IRRERNNIAVRKSRDKAKMCLLELQHKVLELR

SEQ ID NO: 99 - protein, 32 aa, synthetic construct
  CROSSLNK 22..26: note = Intrapeptide stabilizing linkage
  MOD_RES 22: note = (S)-2-(4'-pentenyl)alanine
  MOD_RES 26: note = (S)-2-(4'-pentenyl)alanine
IRRERNNIAVRKSRDKAKMCLXELQXKVLELR

SEQ ID NO: 100 - DNA, 21 bp, synthetic construct
  source 1..21: mol_type = other DNA
cgcttgatgacgtggccggaa

SEQ ID NO: 101 - DNA, 21 bp, synthetic construct
  source 1..21: mol_type = other DNA
cgcttgatgactcagccggaa

SEQ ID NO: 102 - DNA, 21 bp, synthetic construct
  source 1..21: mol_type = other DNA
cgcttgatgacgtggccggaa

SEQ ID NO: 103 - DNA, 21 bp, synthetic construct
  source 1..21: mol_type = other DNA
cgcttgatgaaggggccggaa

SEQ ID NO: 104 - protein, 63 aa, synthetic construct
MKRRIRRERNKMAAAKCRNRRRELTDTLQAETDQLEDEKSALQTEIANLLKEKEKLEFILAAH

SEQ ID NO: 105 - protein, 64 aa, synthetic construct
ERIKAEKRKMRNRIAASKCRKRKLERIARLEEKVKTLKAQNSELASTANMLREQVAQLKQKVMN

SEQ ID NO: 106 - protein, 32 aa, synthetic construct
IRRERNKMAAAKCRNRRRELTDTLQAETDQLE

SEQ ID NO: 107 - protein, 32 aa, synthetic construct
KRKMRNRIAASKCRKRKLERIARLEEKVKTLK

SEQ ID NO: 108 - protein, 24 aa, synthetic construct
MKRRIRRERNKMAAAKCRNRRREL

SEQ ID NO: 109 - protein, 39 aa, synthetic construct
TDTLQAETDQLEDEKSALQTEIANLLKEKEKLEFILAAH

SEQ ID NO: 110 - protein, 26 aa, synthetic construct
ERIKAEKRKMRNRIAASKCRKRKLER

SEQ ID NO: 111 - protein, 38 aa, synthetic construct
IARLEEKVKTLKAQNSELASTANMLREQVAQLKQKVMN

SEQ ID NO: 112 - protein, 32 aa, synthetic construct
SPRKAAAAAARLNRLKKKEYVMGLESRVRGLA

SEQ ID NO: 113 - protein, 32 aa, synthetic construct
RRRGKNKMAAQNSRKRKLDTILNLERDVEDLQ

SEQ ID NO: 114 - protein, 32 aa, synthetic construct
QRREKNRIAAQKCRQRQTQKADTLHLESEDLE

SEQ ID NO: 115 - protein, 32 aa, synthetic construct
RRRSKNRIAAQRCRKRKLDCIQNLESEIRKLV

SEQ ID NO: 116 - protein, 32 aa, synthetic construct
RRRSKNRIAAQRCRKRKLDCIQNLESEIEKLQ

SEQ ID NO: 117 - protein, 32 aa, synthetic construct
RRTLKNRGYAQACRSKRLQQRRGLEAERARLA

SEQ ID NO: 118 - protein, 32 aa, synthetic construct
RRKKEYIDGLESRVAACSAQNQELQKKVQELE

SEQ ID NO: 119 - protein, 32 aa, synthetic construct
RRTLKNRGYAASCRVKRVTQKEELEKQKAELQ

SEQ ID NO: 120 - protein, 32 aa, synthetic construct
RRRGKNKVAAQNCRKRKLENIVELEQDLDHLK

SEQ ID NO: 121 - protein, 32 aa, synthetic construct
EKRRKNNEAAKRSREKRRLNDLVLENKLIALG

SEQ ID NO: 122 - protein, 32 aa, synthetic construct
RRRGKNKVAAQNCRKRKLETIVQLERELERLT

SEQ ID NO: 123 - protein, 32 aa, synthetic construct
RRTLKNRGYAASCRIKRVTQKEELERQRVELQ

SEQ ID NO: 124 - protein, 32 aa, synthetic construct
VRLMKNREAARESRRKKKEYVKSLENRVAVLE

SEQ ID NO: 125 - protein, 32 aa, synthetic construct
RKKKEYMDSLEKKVESCSTENLELRKKVEVLE

SEQ ID NO: 126 - protein, 32 aa, synthetic construct
MAAKRSRDARRLKENQIAIRASFLEKENSALR

SEQ ID NO: 127 - protein, 32 aa, synthetic construct; identical to SEQ ID NO: 106

SEQ ID NO: 128 - protein, 32 aa, synthetic construct
RRTLKNRGYAQSCRFKRVQQRHVLESEKNQLL

SEQ ID NO: 129 - protein, 32 aa, synthetic construct
RRTLKNRGYAASCRVKRVSQKEELQKQKSELE

SEQ ID NO: 130 - protein, 32 aa, synthetic construct
RKRMRNRIAASKSRKRKLERIARLEEKVKTLK

SEQ ID NO: 131 - protein, 32 aa, synthetic construct
RRTLKNRGYAQSSRFKRVQQRHILESEKCQLQ

SEQ ID NO: 132 - protein, 32 aa, synthetic construct
KKKKEYMLGLEARLKAALSENEQLKKENGTLK

SEQ ID NO: 133 - protein, 32 aa, synthetic construct
LRLMKNREAAKESRRRKKEYVKSLESRVAVLE

SEQ ID NO: 134 - protein, 31 aa, synthetic construct
RKKKVYVGGLESRVLKYTAQNMELQNKVQLL

SEQ ID NO: 135 - protein, 32 aa, synthetic construct
IRLMKNREAARECRRKKKEYVKSLENRVAVLE

SEQ ID NO: 136 - protein, 32 aa, synthetic construct
RRREKNRVAAQRSRKKQTQKADKLHEEYESLE

SEQ ID NO: 137 - protein, 32 aa, synthetic construct
RFLERNRAAASRSRQKRKLWVSSLEKKAEELT

SEQ ID NO: 138 - protein, 31 aa, synthetic construct
RQQRMIKNRESASQSRRKKKEYLQGLEARLQ

SEQ ID NO: 139 - protein, 32 aa, synthetic construct
RKKKEYVESLEKKVETFTSENNELWKKVETLE

SEQ ID NO: 140 - protein, 32 aa, synthetic construct
KFLERNRAAATRSRQKRKVWVMSLEKKAEELT

SEQ ID NO: 141 - protein, 32 aa, synthetic construct
LKKQKNRAAAQRSRQKHTDKADALHQQHESLE

SEQ ID NO: 142 - protein, 32 aa, synthetic construct
KKKKEYIDGLETRMSASTAQNQELQRKVLHLE

SEQ ID NO: 143 - protein, 32 aa, synthetic construct
KKMEQNKTAATRYRQKKRAEQEALTGESKELE

SEQ ID NO: 144 - protein, 32 aa, synthetic construct
RRRERNKIAAAKSRNKKKEKTESLQKESEKLE

SEQ ID NO: 145 - protein, 31 aa, synthetic construct
RRERNKLAAAKSRNRRKELTDFLQAETDKLE

SEQ ID NO: 146 - protein, 32 aa, synthetic construct
VRRERNKLAAAKSRNRRRELTDRLQAETDQLE

SEQ ID NO: 147 - protein, 32 aa, synthetic construct
KFLERNRAAASRSRQKRKVWVQSLEKKAEDLS

SEQ ID NO: 148 - protein, 32 aa, synthetic construct
SRRYKNNEAAKRSRDARRLKENQISVRAAFLE

SEQ ID NO: 149 - protein, 32 aa, synthetic construct
RRREKNKVAAARSRNKKKERTEFLQRESERLE

SEQ ID NO: 150 - protein, 32 aa, synthetic construct
RKRLRNRLAATKSRKRKLERIARLEDKVKTLK

SEQ ID NO: 151 - protein, 31 aa, synthetic construct
RKRLRNRIAASKSRKRKLERISRLEKVKTLK

SEQ ID NO: 152 - protein, 32 aa, synthetic construct
IGTTVGRRRRDEQLSPEEEEKRRIRRERNKLA

SEQ ID NO: 153 - protein, 32 aa, synthetic construct
RRTLKNRGYAQSSRYKRVQQKHHLENEKTQLI

SEQ ID NO: 154 - protein, 32 aa, synthetic construct
RRKLKNRVAAQTARDRKKARMSELEQQVVDLE

SEQ ID NO: 155 - protein, 32 aa, synthetic construct
KKRDQNKSAALRYRQRKRAEGEALEGESQGLE

SEQ ID NO: 156 - protein, 32 aa, synthetic construct
IAVRKSRDKAKRRNQEMQQKLVELSAENEKLH

SEQ ID NO: 157 - protein, 32 aa, synthetic construct
MAVKKSRLKSKQKAQDTLQRVNQLKEENERLE

SEQ ID NO: 158 - protein, 32 aa, synthetic construct
YKIRRERNNIAVRKSRDKAKMRNLETQHKVLE

SEQ ID NO: 159 - protein, 32 aa, synthetic construct
IAVRKSRDKAKQRNVETQQKVLELTSDNDRLR

SEQ ID NO: 160 - protein, 32 aa, synthetic construct
YRLRRERNNIAVRKSRDKAKRRILETQQKVLE

SEQ ID NO: 161 - protein, 32 aa, synthetic construct
SDLTPVSELPLTARPRSRKEKNKLASRASRLK

SEQ ID NO: 162 - protein, 32 aa, synthetic construct
SPARAGKQRMKEKEQENERKVAQLAEENERLK

SEQ ID NO: 163 - protein, 32 aa, synthetic construct
DEKYWTRRKKNNVAAKRSRDARRLKENQITIR

SEQ ID NO: 164 - protein, 32 aa, synthetic construct
ESRARKKLRYQYLEELVSSRERAISALREELE

SEQ ID NO: 165 - protein, 32 aa, synthetic construct
RRRGKNKVAAQNSRKRKLDIILNLEDDVSNLQ

SEQ ID NO: 166 - protein, 32 aa, synthetic construct
EKRRKNNEAAKRSREKRRLNDAAIEGRLAALM